(19) United States
(12) Patent Application Publication
(10) Pub. No.: US 2024/0174687 A1
(43) Pub. Date: May 30, 2024

(54) HETEROCYCLIC COMPOUND, ORGANIC LIGHT EMITTING DEVICE COMPRISING SAME AND COMPOSITION FOR ORGANIC LAYER OF ORGANIC LIGHT EMITTING DEVICE

(71) Applicant: LT MATERIALS CO., LTD., Yongin-City (KR)

(72) Inventors: Eui-Jeong CHOI, Yongin-City (KR); Young-Seok NO, Yongin-City (KR); Dong-Jun KIM, Yongin-City (KR)

(73) Assignee: LT MATERIALS CO., LTD., Yongin-City (KR)

(21) Appl. No.: 18/129,310

(22) Filed: Mar. 31, 2023

(30) Foreign Application Priority Data
May 18, 2022 (KR) 10-2022-0060597

(51) Int. Cl. C07D 495/04; C07D 491/04; H10K 50/11; H10K 50/15; H10K 50/16; H10K 50/17; H10K 50/18; H10K 85/60

(52) U.S. Cl. CPC C07D 495/04; C07D 491/04; H10K 50/11; H10K 50/15; H10K 50/16; H10K 50/171; H10K 50/181; H10K 85/6572; C07B 2200/05

(57) ABSTRACT

Disclosed are a heterocyclic compound represented by Chemical Formula 1, and an organic light emitting device and a composition for an organic material layer, including the same.

### CROSS-REFERENCE TO RELATED APPLICATIONS

This application claims priority to and the benefit of Korean Patent Application No. 10-2022-0060597 filed in the Korean Intellectual Property Office on May 18, 2022, the entire contents of which are incorporated herein by reference.

### TECHNICAL FIELD

The present specification relates to a heterocyclic compound, and an organic light emitting device and a composition for an organic material layer, including the same.

### BACKGROUND ART

An organic electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

### Patent Document

(Patent Document 1) U.S. Pat. No. 4,356,429

### SUMMARY OF THE INVENTION

The present invention has been made in an effort to provide a heterocyclic compound, and an organic light emitting device and a composition for an organic material layer, including the same.

An exemplary embodiment of the present invention provides a heterocyclic compound represented by the following Chemical Formula 1.

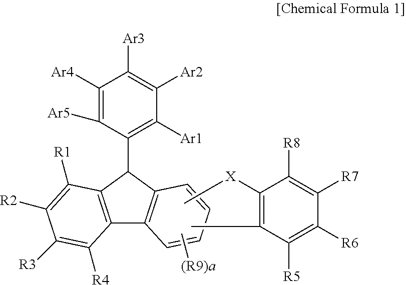

In Chemical Formula 1,
X is O; or S,
Ar1 to Ar5 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group, or adjacent groups are bonded to each other to form a substituted or unsubstituted C6 to C60 aromatic hydrocarbon ring; or a substituted or unsubstituted C2 to C60 aromatic hetero ring,
R1 to R9 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′,
a is an integer from 0 to 2, and when a is 2, substituents in the parenthesis are the same as or different from each other,
at least one of R1, R5, R6 and R8 is represented by the following Chemical Formula A or B,

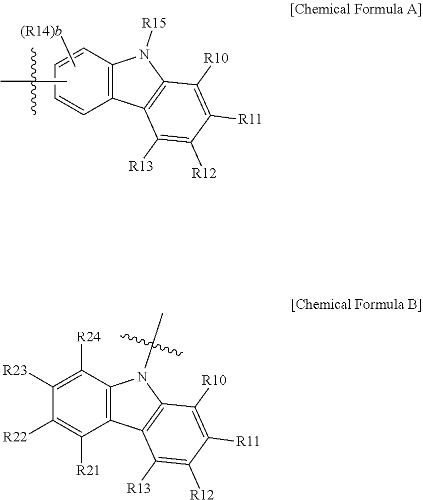

in Chemical Formulae A and B,
R10 to R15 and R21 to R24 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′,
b is an integer from 0 to 3, and when b is 2 or higher, substituents in the parenthesis are the same as or different from each other,
in R1 to R8, at least one of the substituents other than the substituent represented by Chemical Formula A or B is a group represented by —N-Het, and the —N-Het is a substituted or unsubstituted C2 to C60 heteroaryl group including N, and
R, R′ and R″ are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

Another exemplary embodiment provides an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

Yet another exemplary embodiment provides an organic light emitting device in which an organic material layer including the heterocyclic compound of Chemical Formula 1 further includes a heterocyclic compound represented by the following Chemical Formula 2.

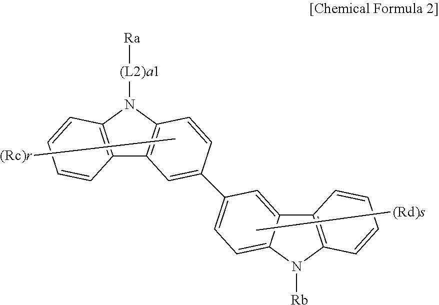

In Chemical Formula 2,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′, or two or more adjacent groups are bonded to each other to form a substituted or unsubstituted aromatic hydrocarbon ring or a substituted or unsubstituted hetero ring,
L2 is a direct bond; a substituted or unsubstituted C6 to C60 arylene group; or a substituted or unsubstituted C2 to C60 heteroarylene group,
Ra and Rb are the same as or different from each other, and are each independently —CN; —SiRR′R″; —P(═O)RR′; a substituted or unsubstituted C6 to C60 aryl group; or a substituted or unsubstituted C2 to C60 heteroaryl group,
R, R′ and R″ are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
a1 is an integer from 0 to 4,
r and s are an integer from 0 to 7, and
when a1, s and r are 2 or higher, substituents in the parenthesis are the same as or different from each other.

Still another exemplary embodiment provides a composition for an organic material layer of an organic light emitting device, which includes the heterocyclic compound represented by Chemical Formula 1 and the heterocyclic compound represented by Chemical Formula 2.

Finally, an exemplary embodiment of the present application provides a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

A heterocyclic compound according to an exemplary embodiment of the present application can be used as a material for an organic material layer of an organic light emitting device. The heterocyclic compound can be used as a material for a hole injection layer, a hole transport layer, a light emitting layer, an electron transport layer, an electron injection layer, a charge generation layer, and the like in an organic light emitting device.

Specifically, the heterocyclic compound represented by Chemical Formula 1 is a compound which has benzofurocarbazole or benzothienocarbazole having a structure in which X is O; or S as a core, and since the heterocyclic compound forms a resonance structure, the substituents of R1, R5, R6 and R8 become relatively electron-rich, and thus have negative charges. In the compound, this site is substituted with Chemical Formula A or B having an unshared pair of electrons, and the HOMO value is increased because the hole characteristics of the molecule are enhanced.

Accordingly, hole traps do not occur in adjacent layers of an OLED including the heterocyclic compound of Chemical Formula 1, and holes are smoothly transferred, thereby improving the driving and service life of the OLED.

Further, both the heterocyclic compound represented by Chemical Formula 1 and the heterocyclic compound represented by Chemical Formula 2 can be used as a material for a light emitting layer of an organic light emitting device. When both the heterocyclic compound represented by Chemical Formula 1 and the heterocyclic compound represented by Chemical Formula 2 are used for the organic light emitting device, the driving voltage of the device can be lowered, the light efficiency of the device can be improved, and the service life characteristics of the device can be improved by the thermal stability of the compound.

### BRIEF DESCRIPTION OF THE DRAWINGS

FIGS. 1 to 3 each are views schematically illustrating a stacking structure of an organic light emitting device according to an exemplary embodiment of the present application.

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

In the present specification,

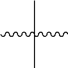

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

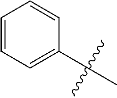 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

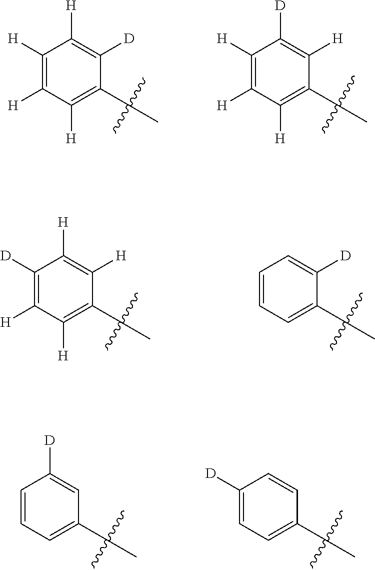 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(=0)₂(R105), and the above-described examples of the alkyl group may be applied to R105.

In the present specification, an arylsulfoxy group is represented by —S(=0)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

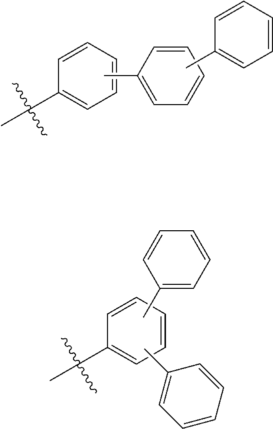

In the present specification, the fluorenyl group may be substituted, and adjacent substituents may be bonded to each other to form a ring.

When the fluorenyl group is substituted, the substituent may be

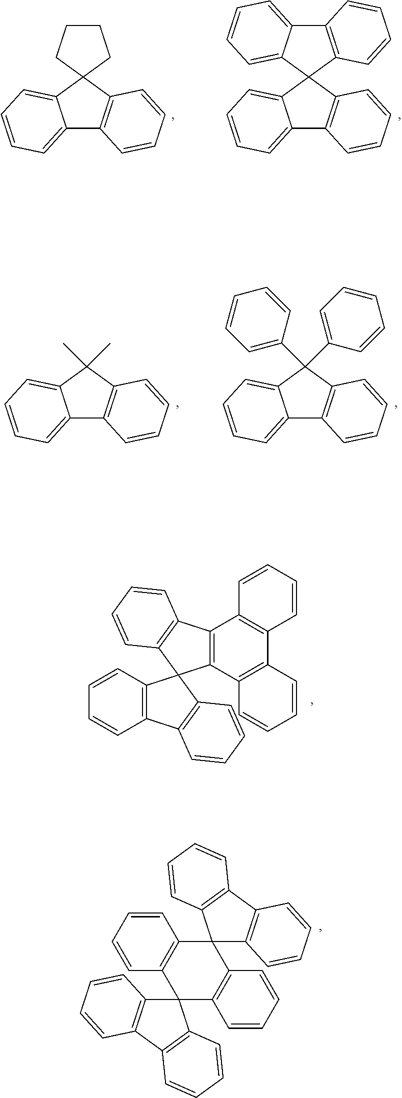

and the like, but is not limited thereto.

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinazoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydrodibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, when the substituent is a carbazole group, it means being bonded to nitrogen or carbon of carbazole.

In the present specification, when a carbazole group is substituted, an additional substituent may be substituted with the nitrogen or carbon of the carbazole.

In the present specification, a benzocarbazole group may be any one of the following structures.

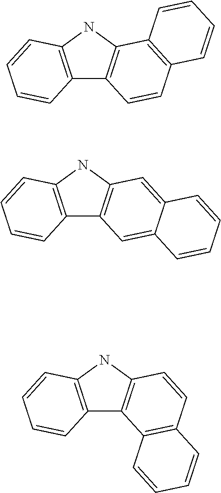 [SMILES: c1ccc2c(c1)ccc1c3ccccc3[nH]c21 ; c1ccc2cc3c(cc2c1)[nH]c1ccccc13 ; c1ccc2c(c1)ccc1[nH]c3ccccc3c12]

In the present specification, a dibenzocarbazole group may be any one of the following structures.

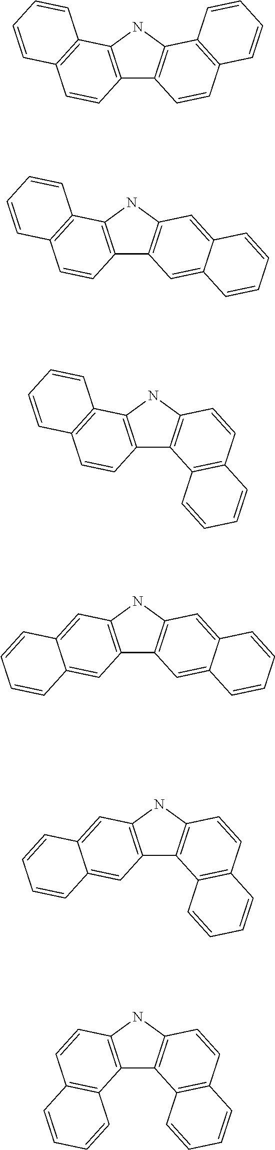 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, a naphthobenzofuran group may be any one of the following structures.

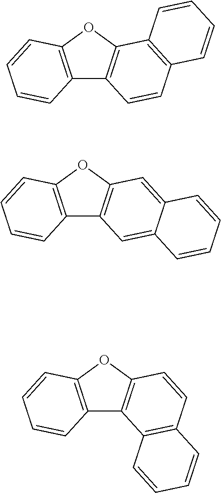 [SMILES: c1ccc2c(c1)ccc1c3ccccc3oc21 ; c1ccc2cc3c(cc2c1)oc1ccccc13 ; c1ccc2c(c1)ccc1oc3ccccc3c12]

In the present specification, a naphthobenzothiophene group may be any one of the following structures.

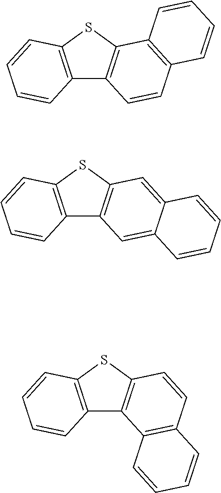 [SMILES: c1ccc2c(c1)ccc1c3ccccc3sc21 ; c1ccc2cc3c(cc2c1)sc1ccccc13 ; c1ccc2c(c1)ccc1sc3ccccc3c12]

In the present specification, a silyl group includes Si and is a substituent to which the Si atom is directly linked as a radical, and is represented by —Si(R107) (R108) (R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include

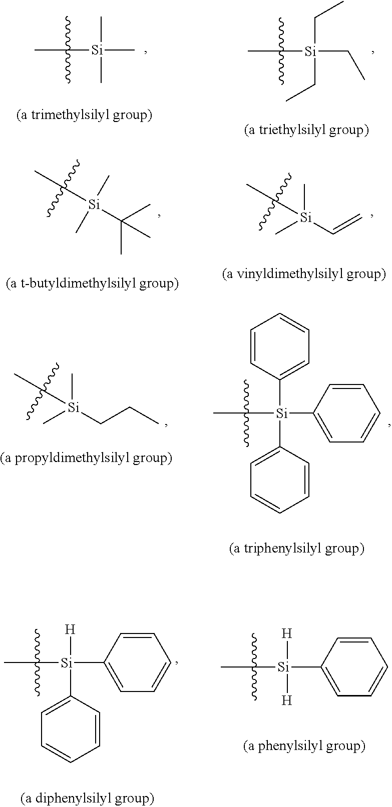

and the like, but are not limited thereto.

In the present specification, a phosphine oxide group is represented by —P(═O) (R110) (R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112) (R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be applied to the rings, except for those that are not monovalent groups.

In an exemplary embodiment of the present application, provided is the heterocyclic compound represented by Chemical Formula 1.

In an exemplary embodiment of the present application, Ar1 to Ar5 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group, or adjacent groups are bonded to each other to form a substituted or unsubstituted C6 to C60 aromatic hydrocarbon ring; or a substituted or unsubstituted C2 to C60 aromatic hetero ring.

In another exemplary embodiment, Ar1 to Ar5 are the same as or different from each other, and are each independently hydrogen; deuterium; a substituted or unsubstituted C1 to C40 alkyl group; a substituted or unsubstituted C6 to C40 aryl group; or a substituted or unsubstituted C2 to C40 heteroaryl group, or adjacent groups are bonded to each other to form a substituted or unsubstituted C6 to C40 aromatic hydrocarbon ring; or a substituted or unsubstituted C2 to C40 aromatic hetero ring.

In still another exemplary embodiment, Ar1 to Ar5 are the same as or different from each other, and are each independently hydrogen; deuterium; a C1 to C40 alkyl group; a C6 to C40 aryl group; or a C2 to C40 heteroaryl group, or adjacent groups are bonded to each other to form a C6 to C40 aromatic hydrocarbon ring; or a C2 to C40 aromatic hetero ring.

In yet another exemplary embodiment, Ar1 to Ar5 are the same as or different from each other, and are each independently hydrogen; deuterium; a C1 to C20 alkyl group; a C6 to C20 aryl group; or a C2 to C20 heteroaryl group.

In still yet another exemplary embodiment, Ar1 to Ar5 are the same as or different from each other, and are each independently hydrogen; or deuterium.

In an exemplary embodiment of the present application, X may be O.

In an exemplary embodiment of the present application, X may be S.

In an exemplary embodiment of the present application, Chemical Formula 1 may be represented by any one of the following Chemical Formulae 3 to 8.

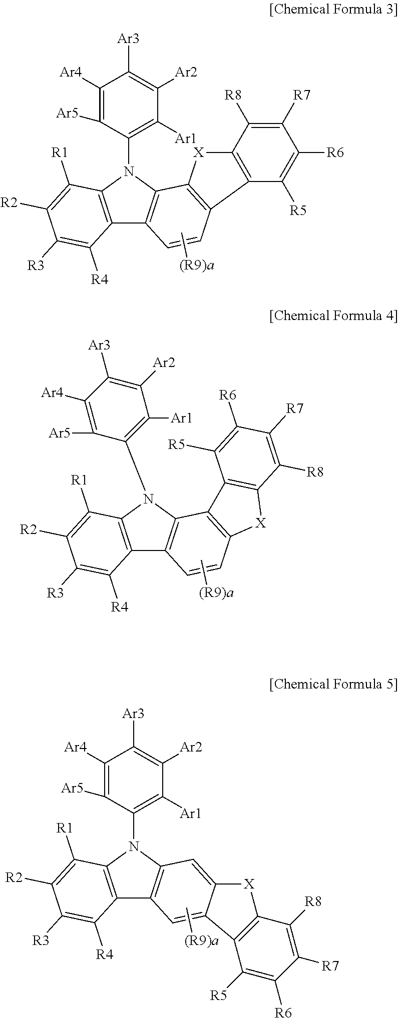

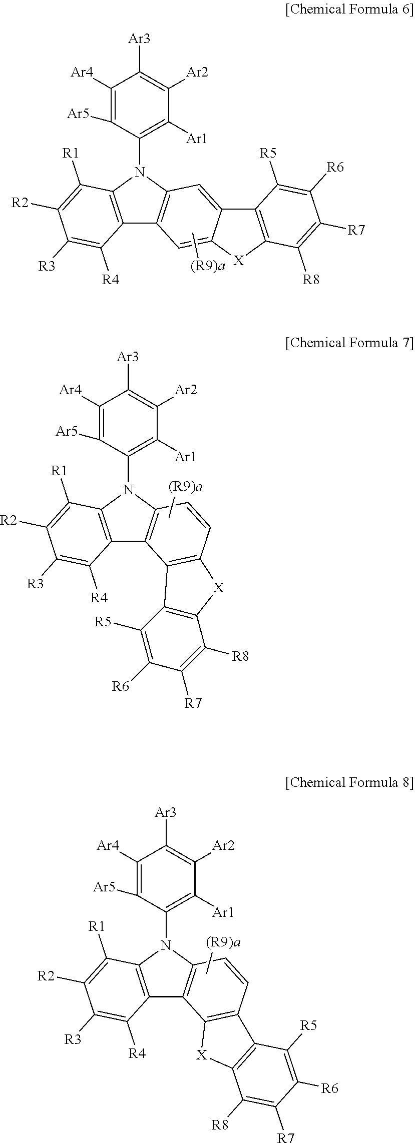

In Chemical Formulae 3 to 8,
the definition of each substituent is the same as the definition in Chemical Formula 1.

In an exemplary embodiment of the present application, at least one of R1, R5, R6 and R8 of Chemical Formula 1 may be represented by Chemical Formula A or B.

In another exemplary embodiment, one of R1, R5, R6 and R8 of Chemical Formula 1 may be represented by Chemical Formula A or B.

In still another exemplary embodiment, R1 of Chemical Formula 1 may be represented by Chemical Formula A or B.

In yet another exemplary embodiment, R5 of Chemical Formula 1 may be represented by Chemical Formula A or B.

In still yet another exemplary embodiment, R6 of Chemical Formula 1 may be represented by Chemical Formula A or B.

In a further exemplary embodiment, R8 of Chemical Formula 1 may be represented by Chemical Formula A or B.

The heterocyclic compound represented by Chemical Formula 1 according to the present application is a compound which has benzofurocarbazole or benzothienocarbazole having a structure in which X is O; or S as a core, and since the heterocyclic compounds forms a resonance structure, the substituents of R1, R5, R6 and R8 become relatively electron-rich, and thus have negative charges. In the compound, this site is substituted with Chemical Formula A or B having an unshared pair of electrons, and the HOMO value is increased because the hole characteristics of the molecule are enhanced, and accordingly, the driving and service life of the organic light emitting device may be improved.

In particular, since the sites of R1 and R8 are adjacent to atoms (O, S and N) that are more electronegative than carbon, the sites of R3 and R6 become relatively electron-richer than the sites of R1 and R8. In this case, nitrogen (N) has one unshared pair of electrons and oxygen (O) has two unshared pairs of electrons, so that the probability that the site of R6 has negative charges is much higher than the position of R3. Therefore, the structure in which the site of R6 is substituted with the substituent of Chemical Formula A or B has a feature in which the driving, efficiency and service life are highly evaluated compared to the case of having the substituent of Chemical Formula A or B at the site of R3.

In an exemplary embodiment of the present application, R10 to R14 and R21 to R24 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′.

In another exemplary embodiment, R10 to R14 and R21 to R24 are the same as or different from each other, and are each independently selected from the group consisting of hydrogen; deuterium; a halogen; a cyano group; a substituted or unsubstituted C1 to C60 alkyl group; a substituted or unsubstituted C6 to C60 aryl group; a substituted or unsubstituted C2 to C60 heteroaryl group; —SiRR′R″; —P(═O)RR′; and —NRR′.

In still another exemplary embodiment, R10 to R14 and R21 to R24 are the same as or different from each other, and are each independently selected from the group consisting of hydrogen; deuterium; a halogen; a cyano group; a substituted or unsubstituted C1 to C40 alkyl group; a substituted or unsubstituted C6 to C40 aryl group; a substituted or unsubstituted C2 to C40 heteroaryl group; —SiRR′R″; —P(═O)RR′; and —NRR′.

In yet another exemplary embodiment, R10 to R14 and R21 to R24 are the same as or different from each other, and are each independently selected from the group consisting of hydrogen; deuterium; a halogen; a cyano group; a C1 to C40 alkyl group; a C6 to C40 aryl group; a C2 to C40 heteroaryl group; —SiRR′R″; —P(═O)RR′; and —NRR′.

In still yet another exemplary embodiment, R10 to R14 and R21 to R24 are the same as or different from each other, and are each independently selected from the group consisting of hydrogen; deuterium; and a C6 to C40 aryl group.

In a further exemplary embodiment, R10 to R14 and R21 to R24 are the same as or different from each other, and are each independently selected from the group consisting of hydrogen; deuterium; and a C6 to C20 aryl group.

In another further exemplary embodiment, R10 to R14 and R21 to R24 are the same as or different from each other, and are each independently selected from the group consisting of hydrogen; deuterium; and a C6 to C10 aryl group.

In still another further exemplary embodiment, R10 to R14 and R21 to R24 are the same as or different from each other, and are each independently selected from the group consisting of hydrogen; deuterium; and a substituted or unsubstituted phenyl group.

In yet another further exemplary embodiment, R10 to R14 and R21 to R24 are the same as or different from each other, and are each independently selected from the group consisting of hydrogen; deuterium; and a phenyl group which is unsubstituted or substituted with deuterium.

In still yet another further exemplary embodiment, R10 to R14 and R21 to R24 are the same as or different from each other, and are each independently selected from the group consisting of hydrogen; deuterium; and a phenyl group.

In an exemplary embodiment of the present application, R15 may be a substituted or unsubstituted C6 to C60 aryl group; or a substituted or unsubstituted C2 to C60 heteroaryl group.

In another exemplary embodiment, R15 may be a substituted or unsubstituted C6 to C40 aryl group; or a substituted or unsubstituted C2 to C40 heteroaryl group.

In still another exemplary embodiment, R15 may be a substituted or unsubstituted C6 to C20 aryl group; or a substituted or unsubstituted C2 to C20 heteroaryl group.

In yet another exemplary embodiment, R15 may be a C6 to C20 aryl group; or a C2 to C20 heteroaryl group.

In still yet another exemplary embodiment, R15 may be a C6 to C10 aryl group; or a C2 to C10 heteroaryl group.

In a further exemplary embodiment, R15 may be a substituted or unsubstituted phenyl group.

In another further exemplary embodiment, R15 may be a phenyl group which is unsubstituted or substituted with deuterium.

In still another further exemplary embodiment, R15 may be a phenyl group.

In an exemplary embodiment of the present application, Chemical Formula A may be represented by any one of the following Chemical Formulae A-1 to A-5.

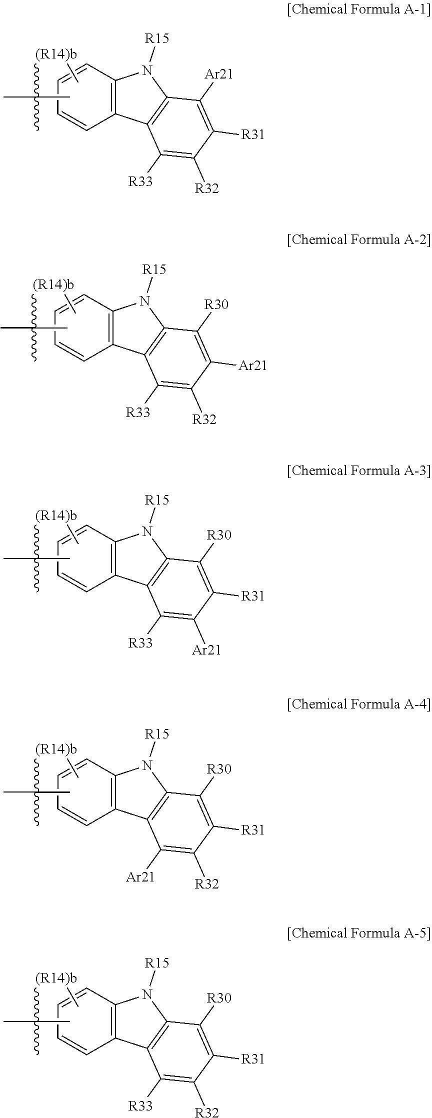

In Chemical Formulae A-1 to A-5,
Ar21 is a substituted or unsubstituted C6 to C60 aryl group; or a substituted or unsubstituted C2 to C60 heteroaryl group,
R30 to R33 are the same as or different from each other, and are each independently hydrogen; or deuterium, and
the definitions of the other substituents are the same as the definitions in Chemical Formula A.

In an exemplary embodiment of the present application, Ar21 may be a substituted or unsubstituted C6 to C60 aryl group; or a substituted or unsubstituted C2 to C60 heteroaryl group.

In another exemplary embodiment, Ar21 may be a substituted or unsubstituted C6 to C40 aryl group; or a substituted or unsubstituted C2 to C40 heteroaryl group.

In still another exemplary embodiment, Ar21 may be a substituted or unsubstituted C6 to C20 aryl group; or a substituted or unsubstituted C2 to C20 heteroaryl group.

In yet another exemplary embodiment, Ar21 may be a C6 to C20 aryl group; or a C2 to C20 heteroaryl group.

In still yet another exemplary embodiment, Ar21 may be a substituted or unsubstituted phenyl group.

In a further exemplary embodiment, Ar21 may be a phenyl group which is unsubstituted or substituted with deuterium.

In another further exemplary embodiment, Ar21 may be a phenyl group.

In an exemplary embodiment of the present application, Chemical Formula B may be represented by any one of the following Chemical Formulae B-1 to B-5.

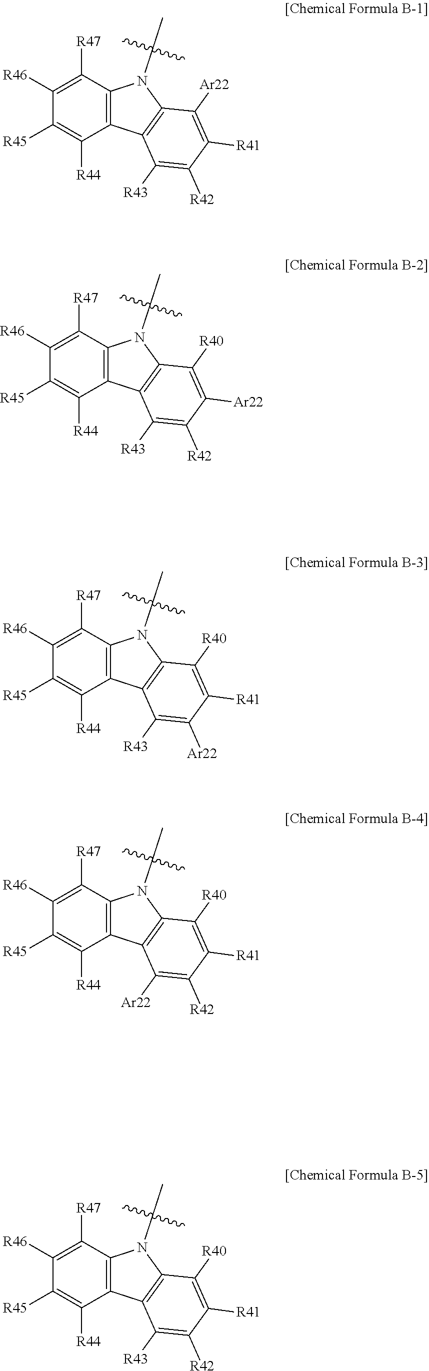

In Chemical Formulae B-1 to B-5,
Ar22 is a substituted or unsubstituted C6 to C60 aryl group; or a substituted or unsubstituted C2 to C60 heteroaryl group, and
R40 to R47 are the same as or different from each other, and are each independently hydrogen; or deuterium.

In an exemplary embodiment of the present application, the definition of Ar22 may be the same as the definition of the above-described Ar21.

In an exemplary embodiment of the present application, provided is a heterocyclic compound in which the

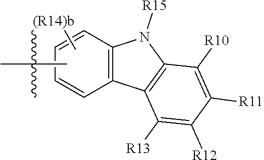

R13 R12 of Chemical Formula A is represented by any one of the following Chemical Formulae 1A to 4A.

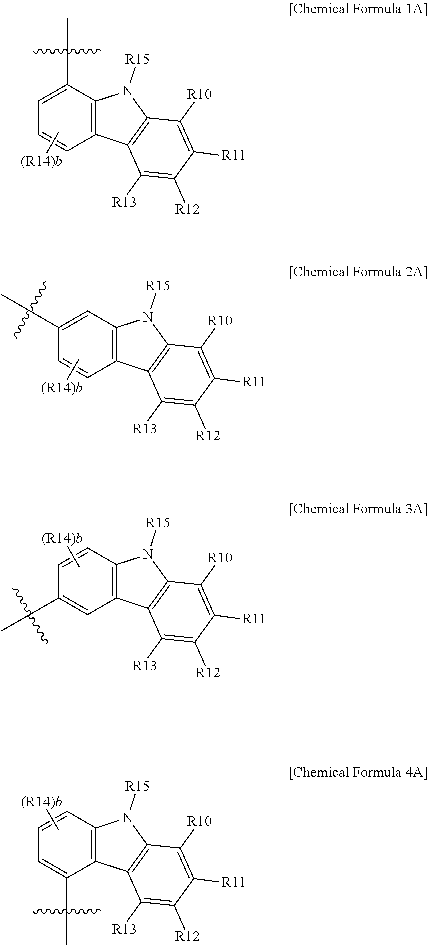

In Chemical Formulae 1A to 4A,

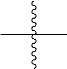

is a position linked to Chemical Formula 1.

In an exemplary embodiment of the present application, in R1 to R8, at least one of the substituents other than the substituent represented by Chemical Formula A or B is a group represented by —N-Het, and the —N-Het is a substituted or unsubstituted C2 to C60 heteroaryl group including N.

In another exemplary embodiment, in R1 to R8, one of the substituents other than the substituent of Chemical Formula A or B is a group represented by —N-Het.

In still another exemplary embodiment, R1 is represented by Chemical Formula A or B, and one of R5 to R8 is a group represented by —N-Het.

In yet another exemplary embodiment, R5 is represented by Chemical Formula A or B, and one of R1 to R4 is a group represented by —N-Het.

In still yet another exemplary embodiment, R6 is represented by Chemical Formula A or B, and one of R1 to R4 is a group represented by —N-Het.

In a further exemplary embodiment, R8 is represented by Chemical Formula A or B, and one of R1 to R4 is a group represented by —N-Het.

In an exemplary embodiment of the present application, R1 to R9 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′.

In an exemplary embodiment of the present application, in R1 to R9, the group represented by Chemical Formula A or B; and the group represented by —N-Het; the other substituents are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′.

In another exemplary embodiment, in R1 to R9, the group represented by Chemical Formula A or B; and the group represented by —N-Het; the other substituents are the same as or different from each other, and are each independently selected from the group consisting of hydrogen; deuterium; a halogen; a cyano group; a substituted or unsubstituted C1 to C60 alkyl group; a substituted or unsubstituted C6 to C60 aryl group; a substituted or unsubstituted C2 to C60 heteroaryl group; —SiRR′R″; —P(═O)RR′; and —NRR′.

In yet another exemplary embodiment, in R1 to R9, the group represented by Chemical Formula A or B; and the group represented by —N-Het; the other substituents are the same as or different from each other, and are each independently selected from the group consisting of hydrogen; deuterium; a halogen; a cyano group; a substituted or unsubstituted C1 to C40 alkyl group; a substituted or unsubstituted C6 to C40 aryl group; a substituted or unsubstituted C2 to C40 heteroaryl group; —SiRR′R″; —P(═O)RR′; and —NRR′.

In still yet another exemplary embodiment, in R1 to R9, the group represented by Chemical Formula A or B; and the group represented by —N-Het; the other substituents are the same as or different from each other, and are each independently selected from the group consisting of hydrogen; deuterium; a halogen; a cyano group; a C1 to C40 alkyl group; a C6 to C40 aryl group; a C2 to C40 heteroaryl group; —SiRR′R″; —P(═O)RR′; and —NRR′.

In a further exemplary embodiment, in R1 to R9, the group represented by Chemical Formula A or B; and the group represented by —N-Het; the other substituents are the same as or different from each other, and may be each independently hydrogen; or deuterium.

In an exemplary embodiment of the present application, the group represented by —N-Het is a substituted or unsubstituted C2 to C60 heteroaryl group including N.

In another exemplary embodiment, the group represented by —N-Het is a substituted or unsubstituted C2 to C40 heteroaryl group including N.

In still another exemplary embodiment, the group represented by —N-Het is a C2 to C40 heteroaryl group including N, which is unsubstituted or substituted with one or more substituents selected from the group consisting of deuterium and a C6 to C40 aryl group.

In yet another exemplary embodiment, the group represented by —N-Het may be a pyridine group which is unsubstituted or substituted with one or more substituents selected from the group consisting of deuterium and a C6 to C40 aryl group; a pyrimidine group which is unsubstituted or substituted with one or more substituents selected from the group consisting of deuterium and a C6 to C40 aryl group; a triazine group which is unsubstituted or substituted with one or more substituents selected from the group consisting of deuterium and a C6 to C40 aryl group; a quinoline group which is unsubstituted or substituted with one or more substituents selected from the group consisting of deuterium and a C6 to C40 aryl group; a quinazoline group which is unsubstituted or substituted with one or more substituents selected from the group consisting of deuterium and a C6 to C40 aryl group; or a phenanthroline group which is unsubstituted or substituted with one or more substituents selected from the group consisting of deuterium and a C6 to C40 aryl group.

In still yet another exemplary embodiment, the group represented by —N-Het may be a pyridine group which is unsubstituted or substituted with one or more substituents selected from the group consisting of deuterium, a phenyl group, a biphenyl group and a terphenyl group; a pyrimidine group which is unsubstituted or substituted with one or more substituents selected from the group consisting of deuterium, a phenyl group, a biphenyl group and a terphenyl group; a triazine group which is unsubstituted or substituted with one or more substituents selected from the group consisting of deuterium, a phenyl group, a biphenyl group and a terphenyl group; a quinoline group which is unsubstituted or substituted with one or more substituents selected from the group consisting of deuterium, a phenyl group, a biphenyl group and a terphenyl group; a quinazoline group which is unsubstituted or substituted with one or more substituents selected from the group consisting of deuterium, a phenyl group, a biphenyl group and a terphenyl group; or a phenanthroline group which is unsubstituted or substituted with one or more substituents selected from the group consisting of deuterium, a phenyl group, a biphenyl group and a terphenyl group.

In an exemplary embodiment of the present application, the —N-Het may be represented by the following Chemical Formula C.

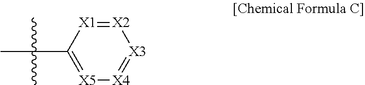

In Chemical Formula C,
X1 is N or CR51, X2 is N or CR52, X3 is N or CR53, X4 is N or CR54, X5 is N or CR55, and at least one of X1 to X5 is N, and
R51 to R55 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group, or adjacent groups are bonded to each other to form a substituted or unsubstituted C6 to C6 aromatic hydrocarbon ring; or a substituted or unsubstituted C2 to C60 aromatic hetero ring.

In an exemplary embodiment of the present application, R51 to R55 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group, or adjacent groups are bonded to each other to form a substituted or unsubstituted C6 to C60 aromatic hydrocarbon ring; or a substituted or unsubstituted C2 to C60 aromatic hetero ring.

In an exemplary embodiment of the present application, R51 to R55 are the same as or different from each other, and are each independently hydrogen; deuterium; a substituted or unsubstituted C1 to C40 alkyl group; a substituted or unsubstituted C6 to C40 aryl group; or a substituted or unsubstituted C2 to C40 heteroaryl group, or adjacent groups are bonded to each other to form a substituted or unsubstituted C6 to C40 aromatic hydrocarbon ring; or a substituted or unsubstituted C2 to C40 aromatic hetero ring.

In an exemplary embodiment of the present application, R51 to R55 are the same as or different from each other, and are each independently hydrogen; deuterium; a C1 to C40 alkyl group; a C6 to C40 aryl group; or a C2 to C40 heteroaryl group, or adjacent groups are bonded to each other to form a C6 to C40 aromatic hydrocarbon ring; or a C2 to C40 aromatic hetero ring.

In an exemplary embodiment of the present application, R51 to R55 are the same as or different from each other, and may be each independently hydrogen; deuterium; a substituted or unsubstituted phenyl group; a substituted or unsubstituted biphenyl group; or a substituted or unsubstituted terphenyl group.

In an exemplary embodiment of the present application, R51 to R55 are the same as or different from each other, and may be each independently hydrogen; deuterium; a phenyl group which is unsubstituted or substituted with deuterium; a biphenyl group which is unsubstituted or substituted with deuterium; or a terphenyl group which is unsubstituted or substituted with deuterium.

In an exemplary embodiment of the present application, the deuterium content of Chemical Formula 1 may be 0% or more and 100% or less.

In another exemplary embodiment, the deuterium content of Chemical Formula 1 may be 0%, or 50% or more and 100% or less.

In still another exemplary embodiment, the deuterium content of Chemical Formula 1 may be 0%, or 60% or more and 100% or less.

In yet another exemplary embodiment, the deuterium content of Chemical Formula 1 may be 0%, or 70% or more and 100% or less.

In still yet another exemplary embodiment, the deuterium content of Chemical Formula 1 may be 0%, or 80% or more and 100% or less.

In a further exemplary embodiment, the deuterium content of Chemical Formula 1 may be 0% or 100%.

In an exemplary embodiment of the present application, R, R′, and R″ are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In another exemplary embodiment, R, R′, and R″ are the same as or different from each other, and may be each independently a substituted or unsubstituted C1 to C60 alkyl group; or a substituted or unsubstituted C6 to C60 aryl group.

In still another exemplary embodiment, R, R′, and R″ are the same as or different from each other, and may be each independently a C1 to C60 alkyl group; or a C6 to C60 aryl group.

In yet another exemplary embodiment, R, R′, and R″ are the same as or different from each other, and may be each independently a methyl group; or a phenyl group.

In still yet another exemplary embodiment, R, R′, and R″ may be a substituted or unsubstituted methyl group.

In a further exemplary embodiment, R, R′, and R″ may be a substituted or unsubstituted phenyl group.

In another further exemplary embodiment, R, R′, and R″ may be a phenyl group.

In still another further exemplary embodiment, R, R′, and R″ may be a methyl group.

In an exemplary embodiment of the present application, provided is a heterocyclic compound in which Chemical Formula 1 is represented by any one of the following compounds. Further, in an exemplary embodiment of the present application, the following compound is just one example and is not limited thereto, and may include other compounds included in Chemical Formula 1 which includes an additional substituent. That is, regarding the substitution position of deuterium in the following compound, specific positions are excluded during the process of deuterium substitution and synthesis as long as only the above-described content of deuterium is satisfied, and hydrogen and deuterium may be present in a mixed state.

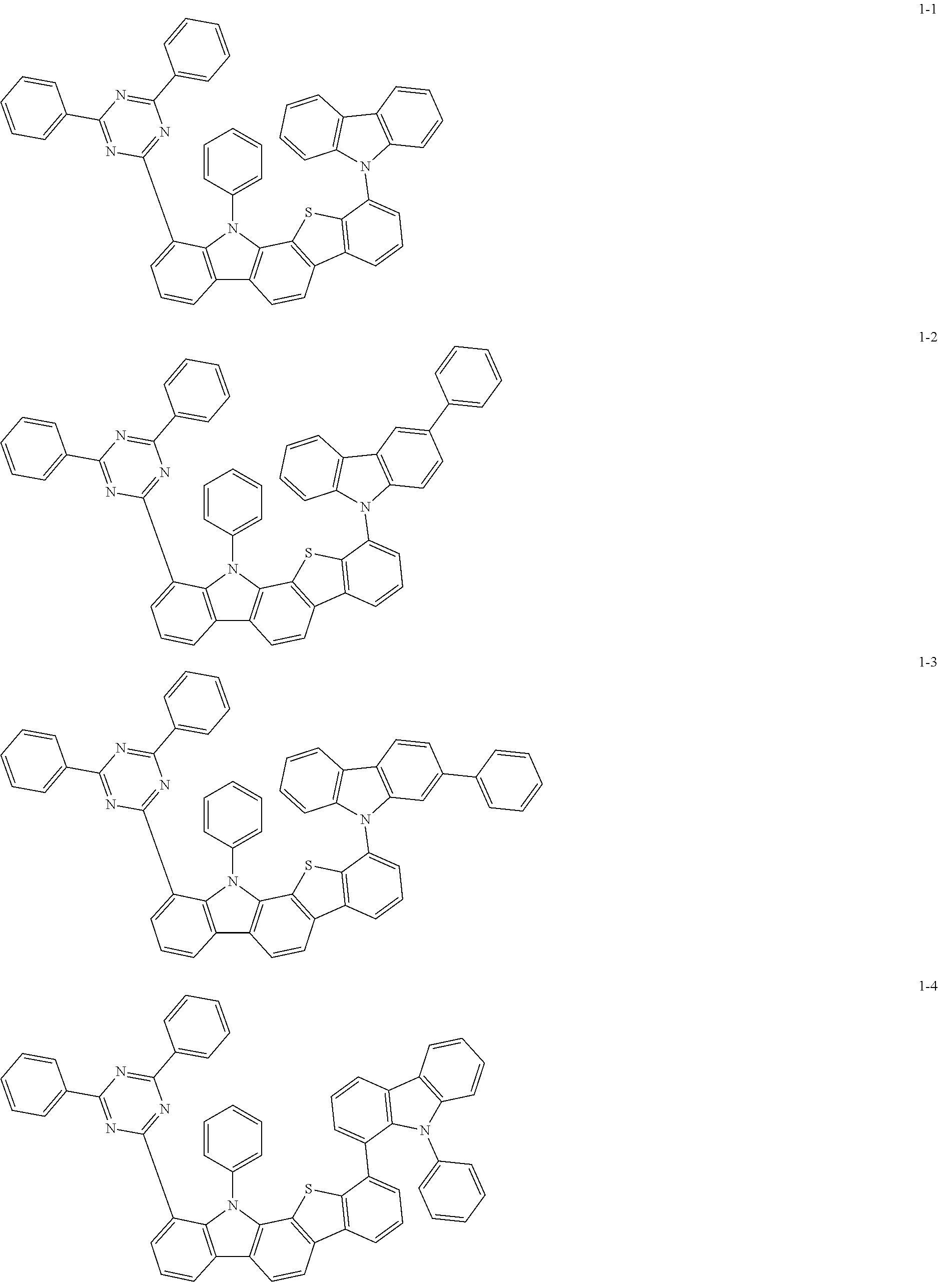

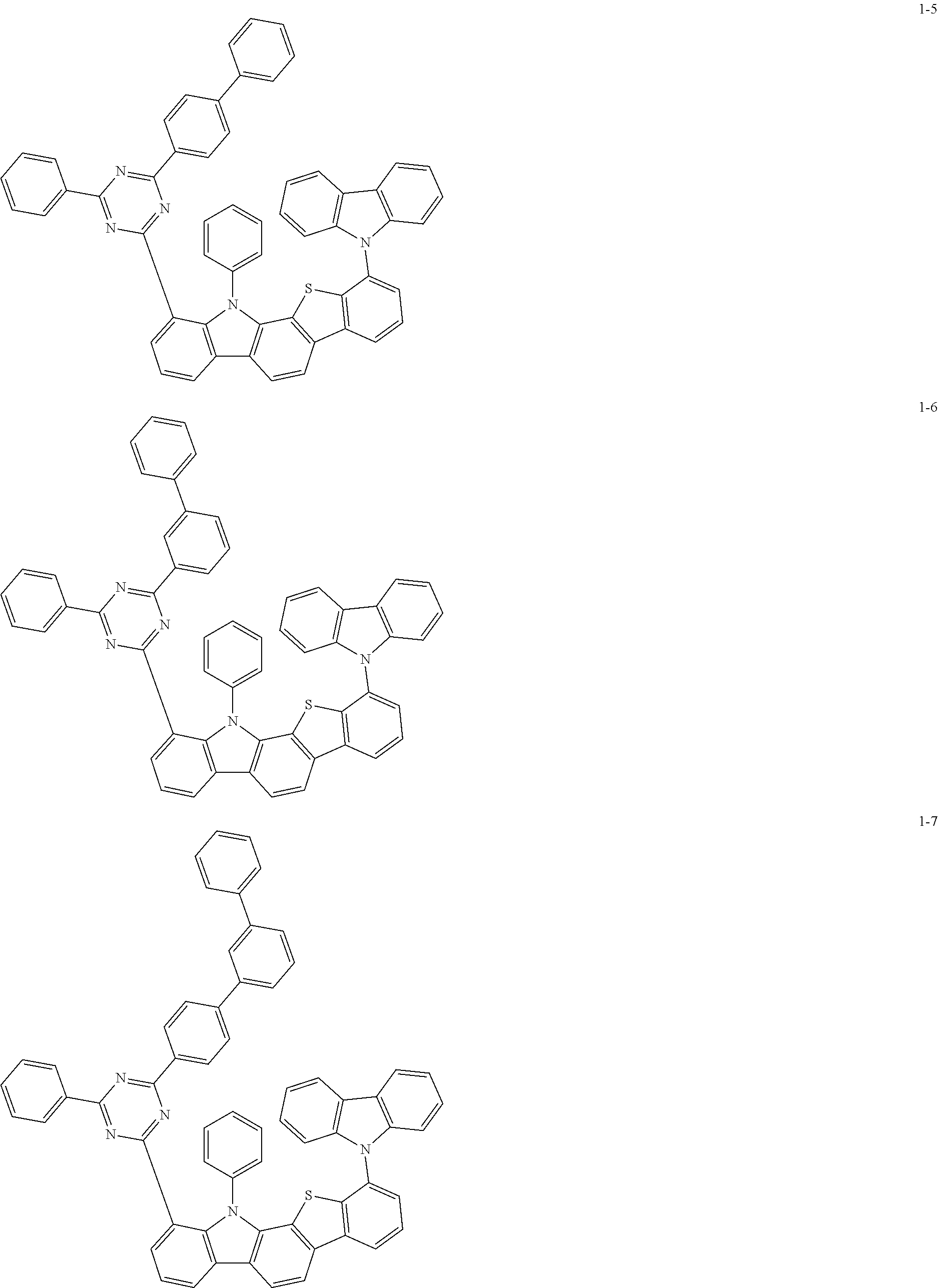

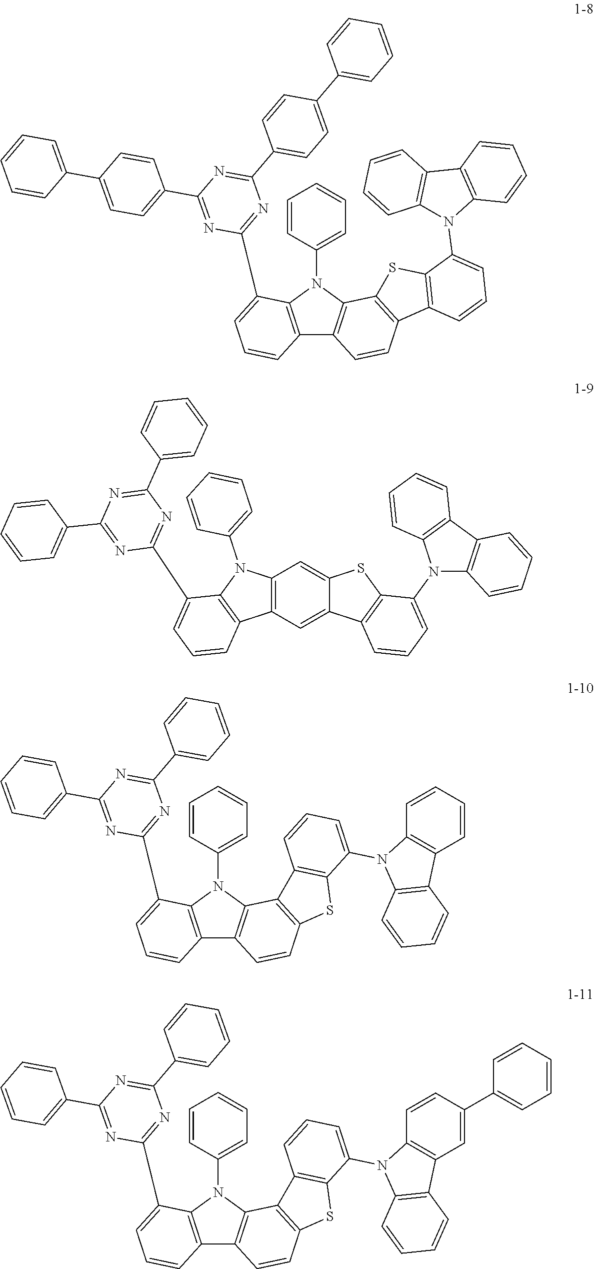

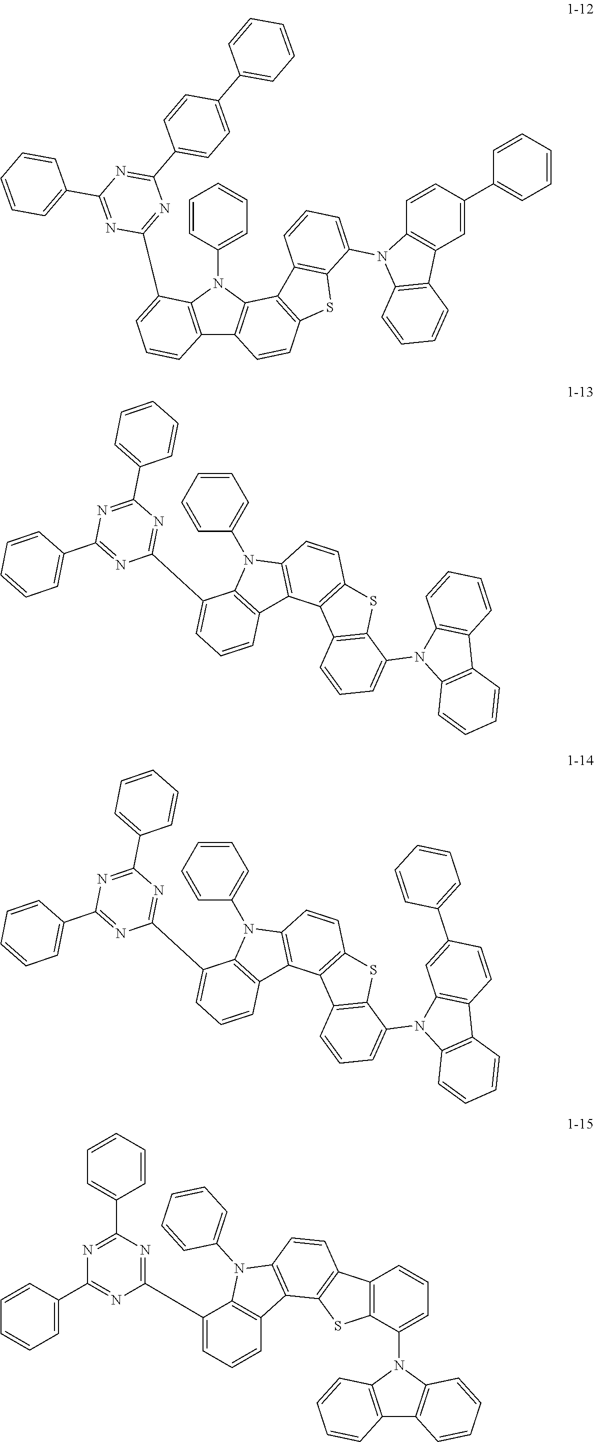 [SMILES: c1ccc(-c2ccc(-c3nc(-c4ccccc4)nc(-c4cccc5c6ccc7sc8c(-n9c%10ccccc%10c%10cc(-c%11ccccc%11)ccc%109)cccc8c7c6n(-c6ccccc6)c45)n3)cc2)cc1 ; c1ccc(-c2nc(-c3ccccc3)nc(-c3cccc4c5c6c(ccc5n(-c5ccccc5)c34)sc3c(-n4c5ccccc5c5ccccc54)cccc36)n2)cc1 ; c1ccc(-c2ccc3c4ccccc4n(-c4cccc5c4sc4ccc6c(c7cccc(-c8nc(-c9ccccc9)nc(-c9ccccc9)n8)c7n6-c6ccccc6)c45)c3c2)cc1 ; c1ccc(-c2nc(-c3ccccc3)nc(-c3cccc4c5c6sc7c(-n8c9ccccc9c9ccccc98)cccc7c6ccc5n(-c5ccccc5)c34)n2)cc1]

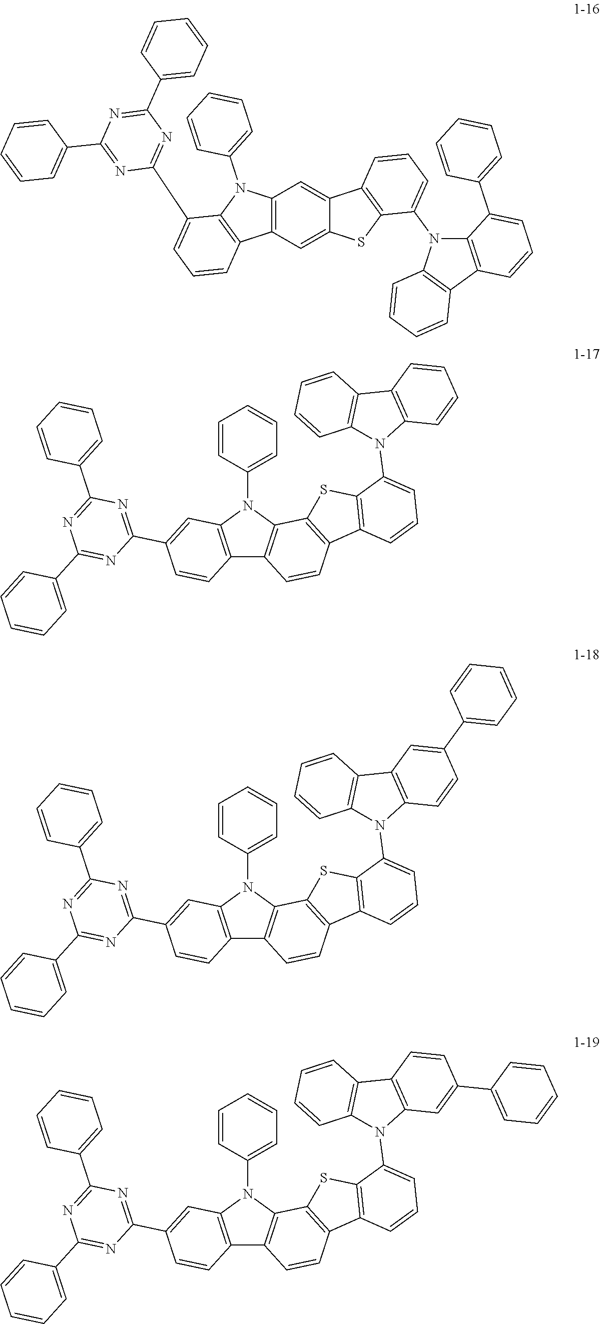

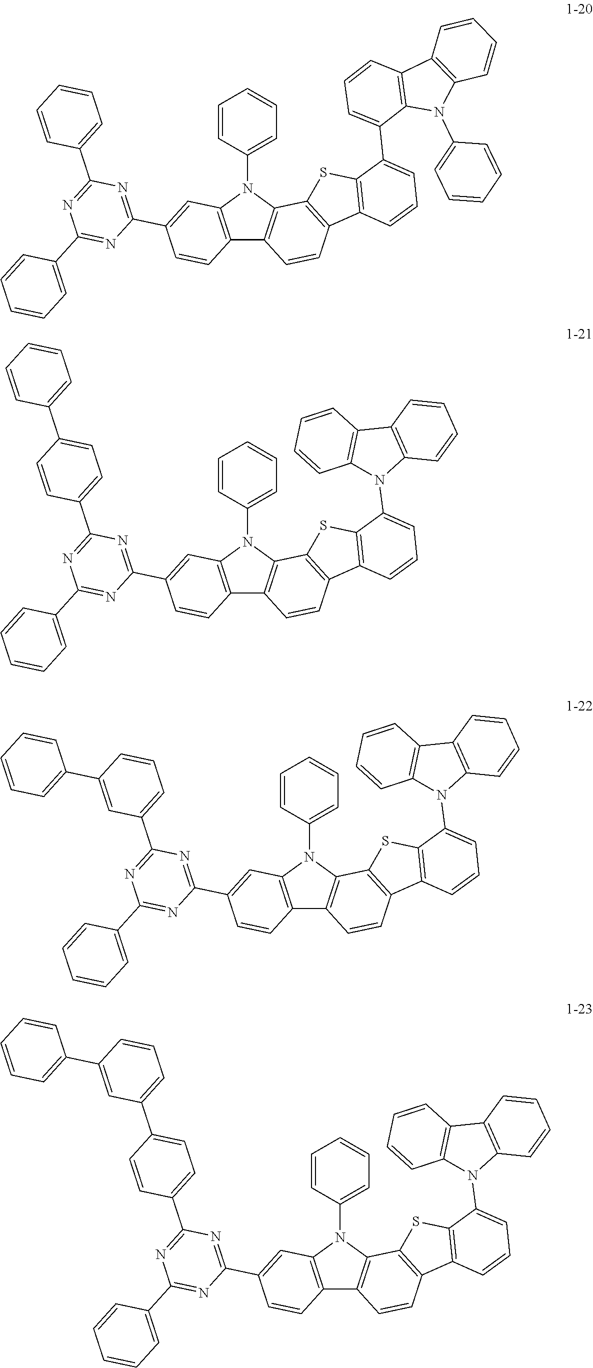

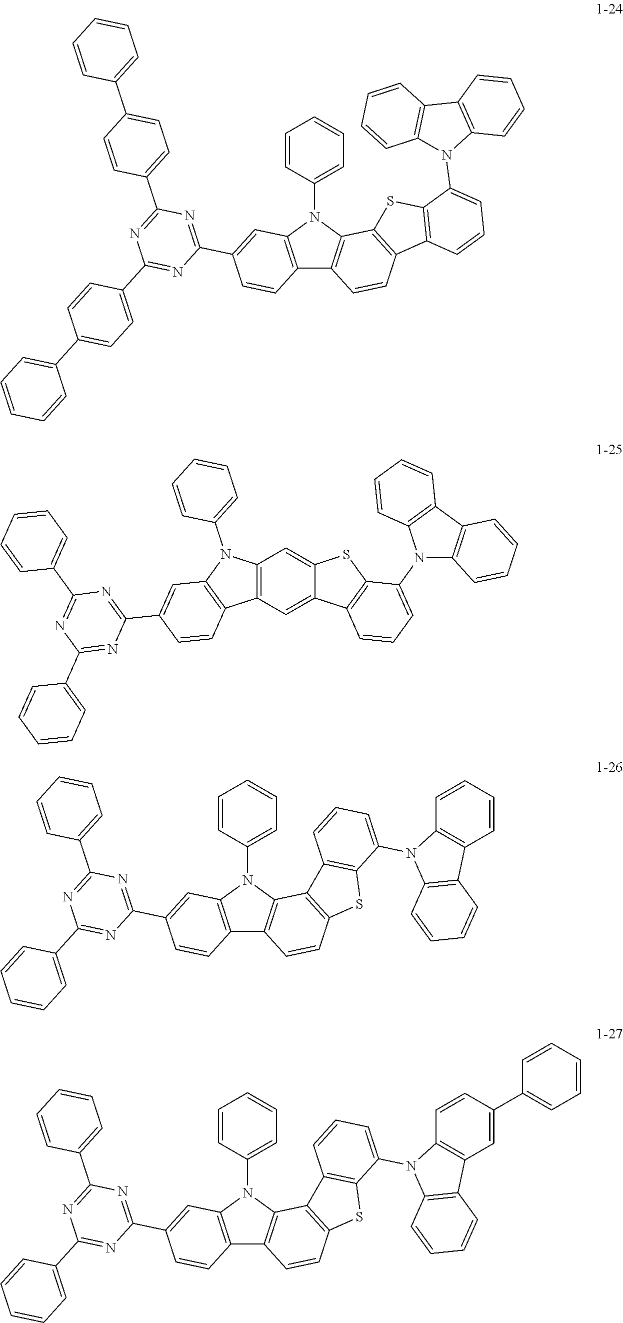

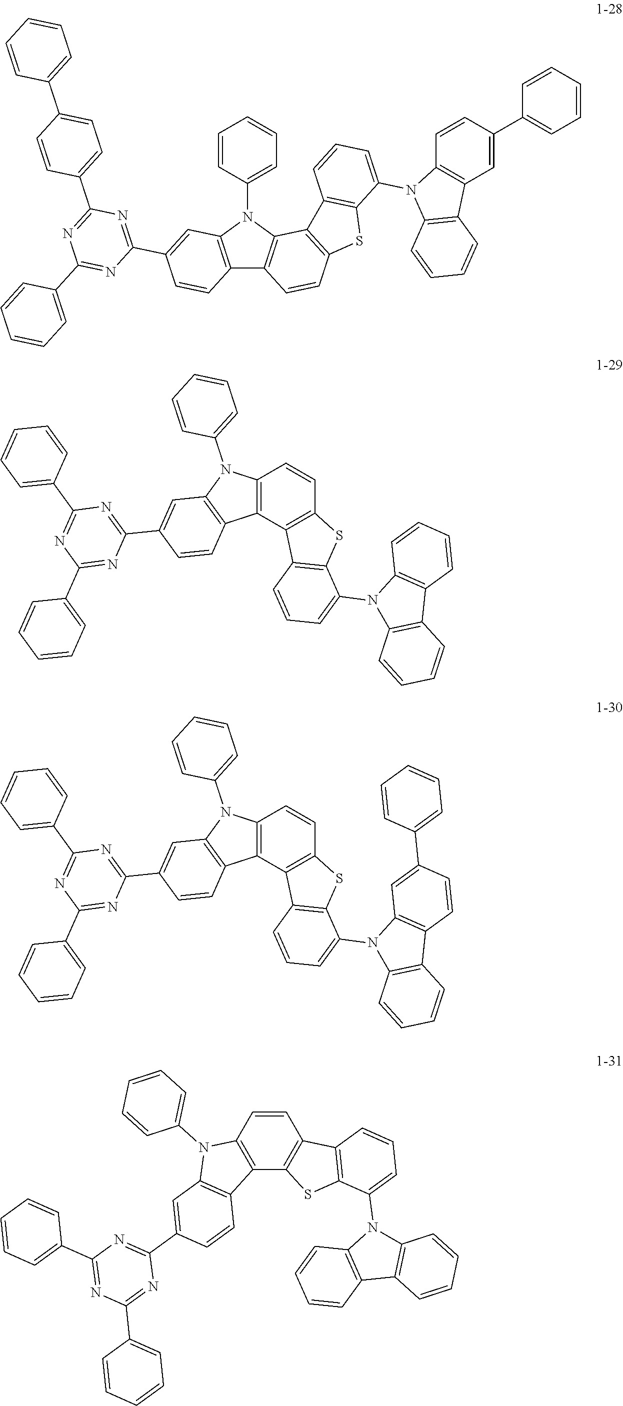 [SMILES: c1ccc(-c2ccc(-c3nc(-c4ccccc4)nc(-c4ccc5c6ccc7sc8c(-n9c%10ccccc%10c%10cc(-c%11ccccc%11)ccc%109)cccc8c7c6n(-c6ccccc6)c5c4)n3)cc2)cc1 ; c1ccc(-c2nc(-c3ccccc3)nc(-c3ccc4c5c6c(ccc5n(-c5ccccc5)c4c3)sc3c(-n4c5ccccc5c5ccccc54)cccc36)n2)cc1 ; c1ccc(-c2ccc3c4ccccc4n(-c4cccc5c4sc4ccc6c(c7ccc(-c8nc(-c9ccccc9)nc(-c9ccccc9)n8)cc7n6-c6ccccc6)c45)c3c2)cc1 ; c1ccc(-c2nc(-c3ccccc3)nc(-c3ccc4c5c6sc7c(-n8c9ccccc9c9ccccc98)cccc7c6ccc5n(-c5ccccc5)c4c3)n2)cc1]

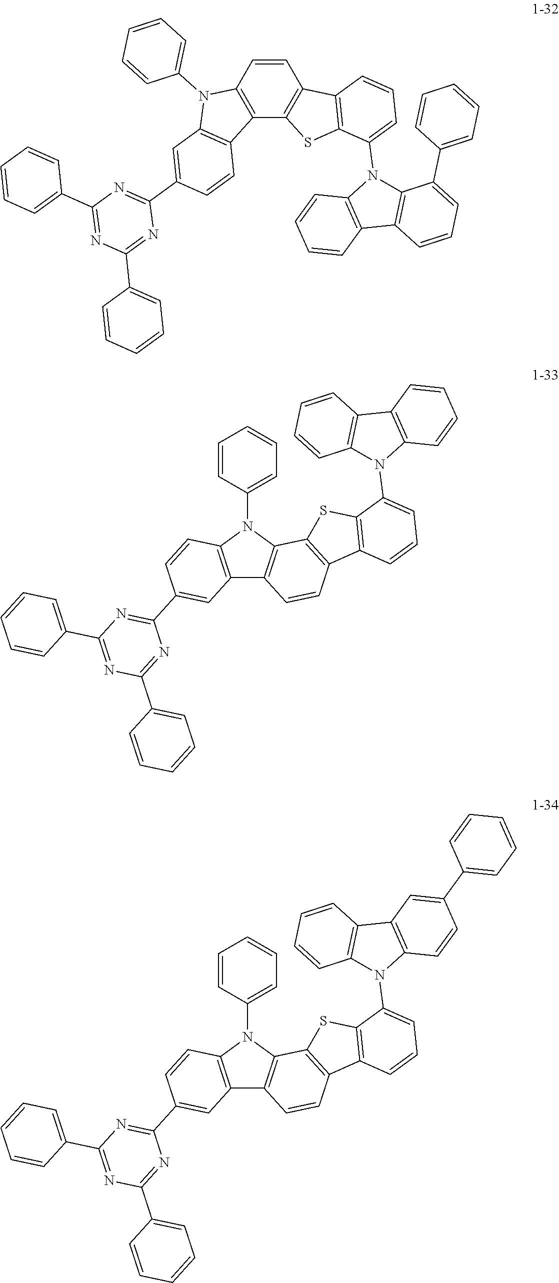 [SMILES: c1ccc(-c2nc(-c3ccccc3)nc(-c3ccc4c5c6sc7c(-n8c9ccccc9c9cccc(-c%10ccccc%10)c98)cccc7c6ccc5n(-c5ccccc5)c4c3)n2)cc1 ; c1ccc(-c2nc(-c3ccccc3)nc(-c3ccc4c(c3)c3ccc5c6cccc(-n7c8ccccc8c8ccccc87)c6sc5c3n4-c3ccccc3)n2)cc1 ; c1ccc(-c2ccc3c(c2)c2ccccc2n3-c2cccc3c2sc2c3ccc3c4cc(-c5nc(-c6ccccc6)nc(-c6ccccc6)n5)ccc4n(-c4ccccc4)c32)cc1]

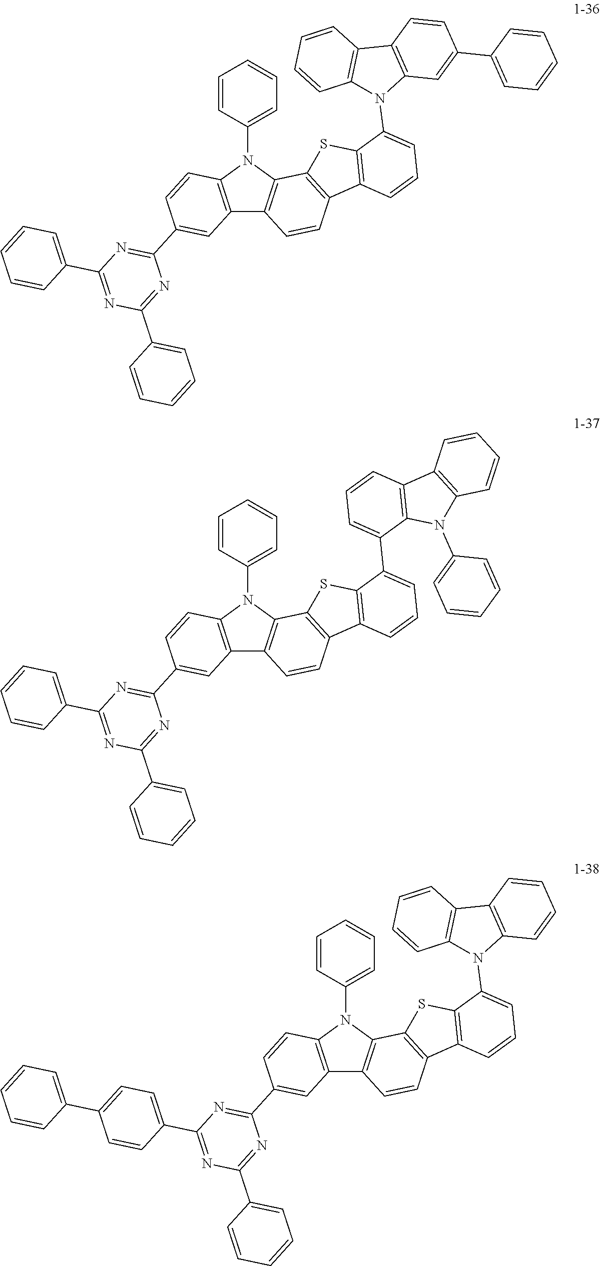 [SMILES: c1ccc(-c2ccc3c4ccccc4n(-c4cccc5c4sc4c5ccc5c6cc(-c7nc(-c8ccccc8)nc(-c8ccccc8)n7)ccc6n(-c6ccccc6)c54)c3c2)cc1 ; c1ccc(-c2nc(-c3ccccc3)nc(-c3ccc4c(c3)c3ccc5c6cccc(-c7cccc8c9ccccc9n(-c9ccccc9)c78)c6sc5c3n4-c3ccccc3)n2)cc1 ; c1ccc(-c2ccc(-c3nc(-c4ccccc4)nc(-c4ccc5c(c4)c4ccc6c7cccc(-n8c9ccccc9c9ccccc98)c7sc6c4n5-c4ccccc4)n3)cc2)cc1]

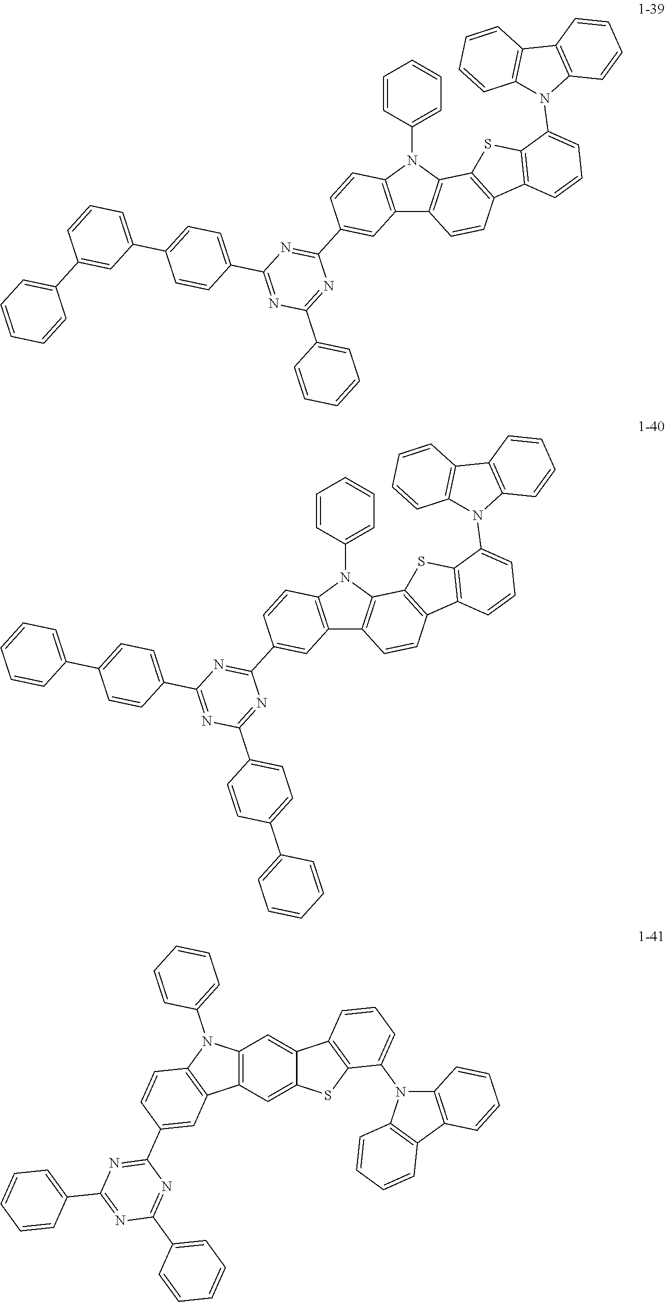 [SMILES: c1ccc(-c2cccc(-c3ccc(-c4nc(-c5ccccc5)nc(-c5ccc6c(c5)c5ccc7c8cccc(-n9c%10ccccc%10c%10ccccc%109)c8sc7c5n6-c5ccccc5)n4)cc3)c2)cc1 ; c1ccc(-c2ccc(-c3nc(-c4ccc(-c5ccccc5)cc4)nc(-c4ccc5c(c4)c4ccc6c7cccc(-n8c9ccccc9c9ccccc98)c7sc6c4n5-c4ccccc4)n3)cc2)cc1 ; c1ccc(-c2nc(-c3ccccc3)nc(-c3ccc4c(c3)c3cc5sc6c(-n7c8ccccc8c8ccccc87)cccc6c5cc3n4-c3ccccc3)n2)cc1]

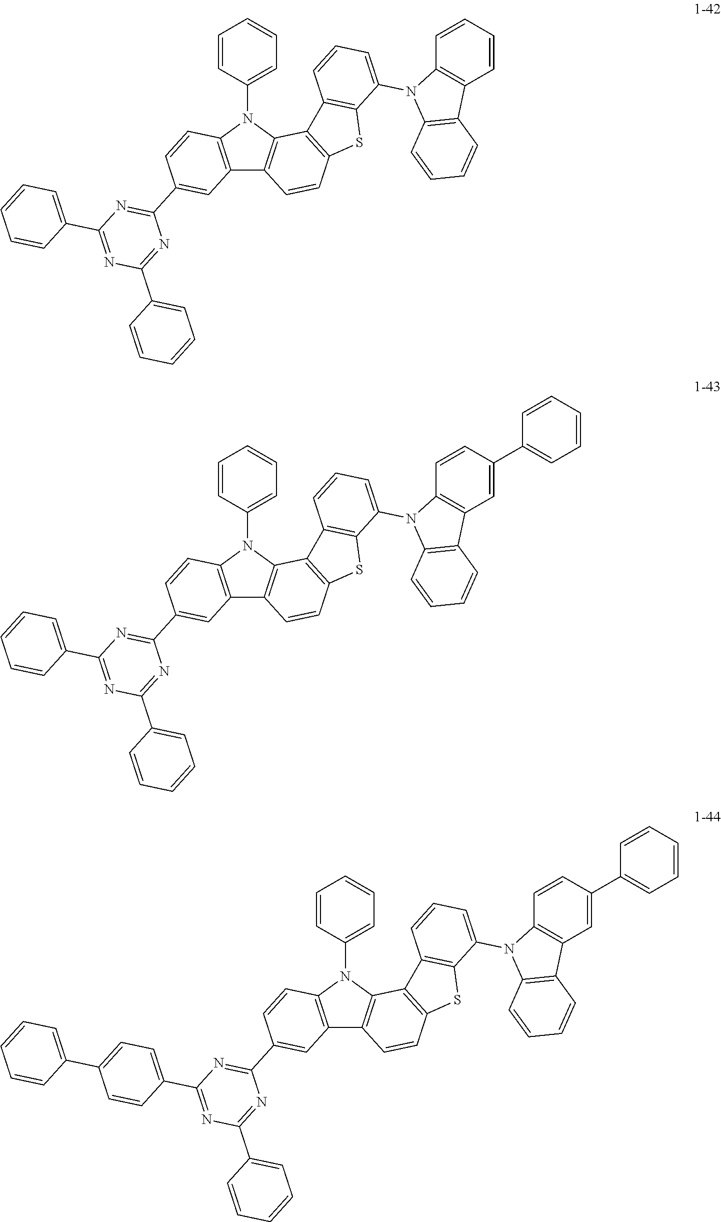 [SMILES: c1ccc(-c2nc(-c3ccccc3)nc(-c3ccc4c(c3)c3ccc5sc6c(-n7c8ccccc8c8ccccc87)cccc6c5c3n4-c3ccccc3)n2)cc1 ; c1ccc(-c2ccc3c(c2)c2ccccc2n3-c2cccc3c2sc2ccc4c5cc(-c6nc(-c7ccccc7)nc(-c7ccccc7)n6)ccc5n(-c5ccccc5)c4c23)cc1 ; c1ccc(-c2ccc(-c3nc(-c4ccccc4)nc(-c4ccc5c(c4)c4ccc6sc7c(-n8c9ccccc9c9cc(-c%10ccccc%10)ccc98)cccc7c6c4n5-c4ccccc4)n3)cc2)cc1]

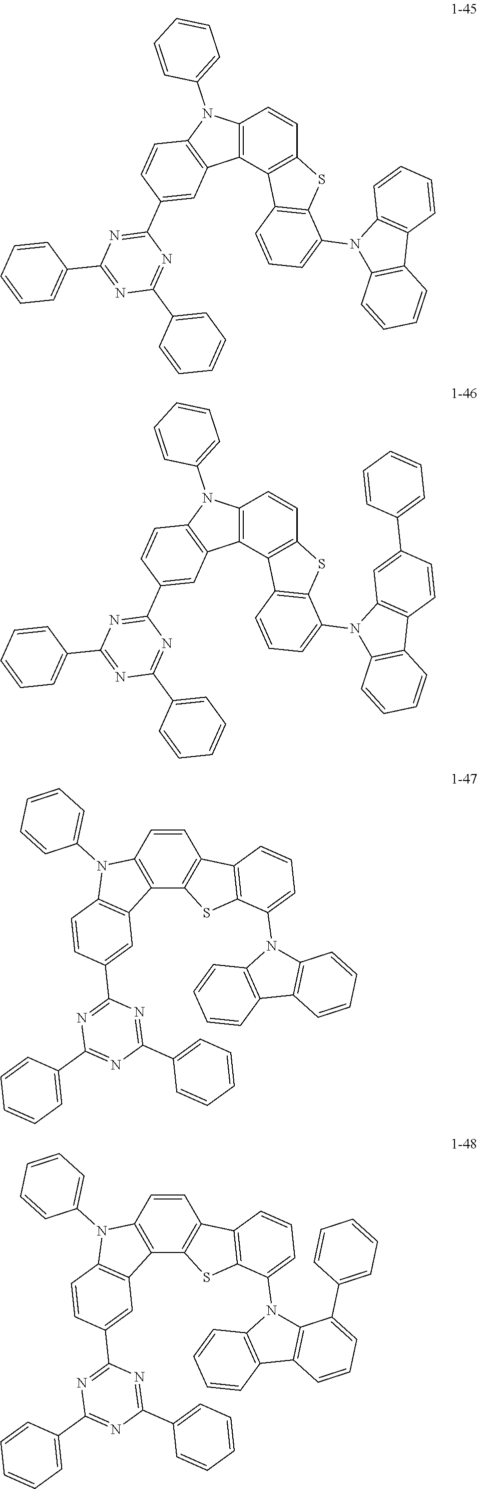

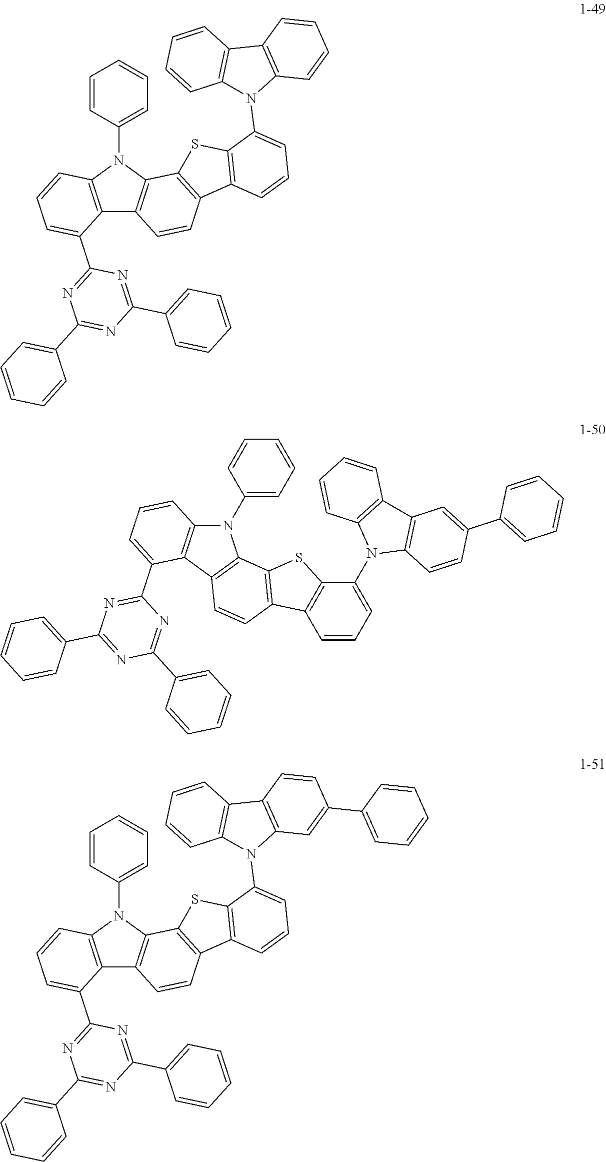 [SMILES: c1ccc(-c2nc(-c3ccccc3)nc(-c3cccc4c3c3ccc5c6cccc(-n7c8ccccc8c8ccccc87)c6sc5c3n4-c3ccccc3)n2)cc1 ; c1ccc(-c2ccc3c(c2)c2ccccc2n3-c2cccc3c2sc2c3ccc3c4c(-c5nc(-c6ccccc6)nc(-c6ccccc6)n5)cccc4n(-c4ccccc4)c32)cc1 ; c1ccc(-c2ccc3c4ccccc4n(-c4cccc5c4sc4c5ccc5c6c(-c7nc(-c8ccccc8)nc(-c8ccccc8)n7)cccc6n(-c6ccccc6)c54)c3c2)cc1]

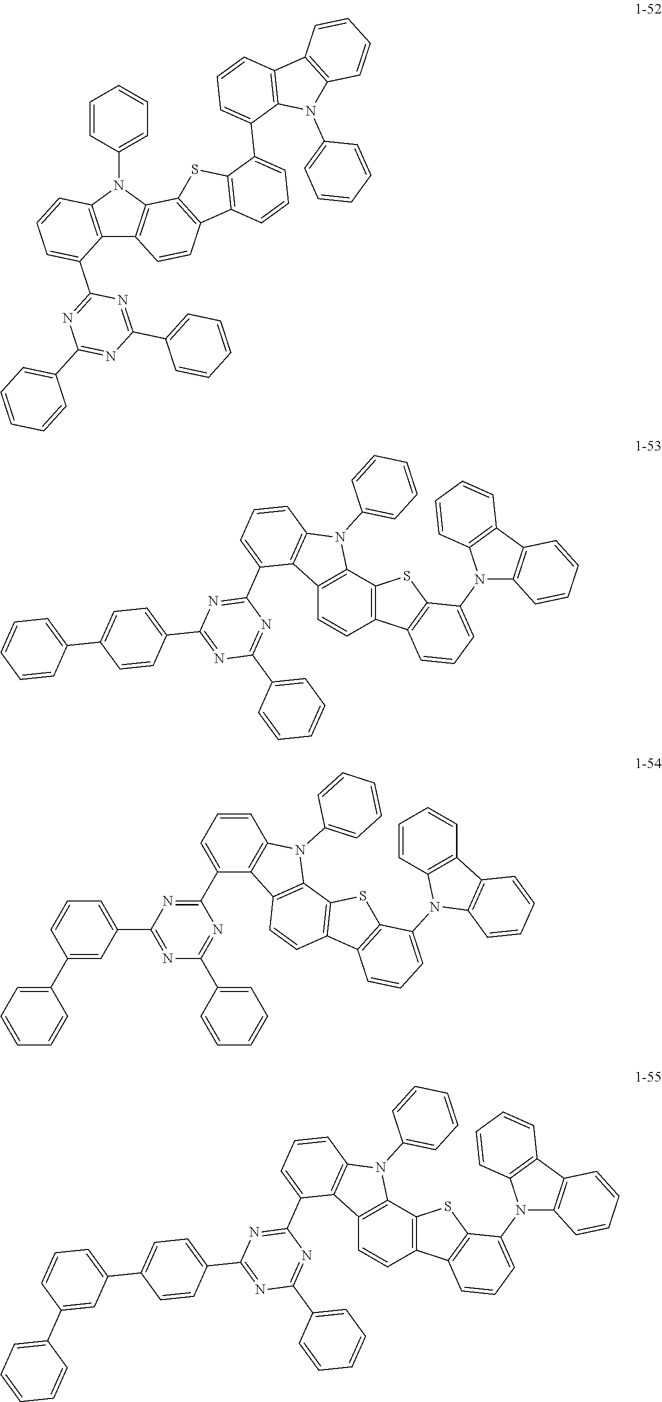 [SMILES: c1ccc(-c2nc(-c3ccccc3)nc(-c3cccc4c3c3ccc5c6cccc(-c7cccc8c9ccccc9n(-c9ccccc9)c78)c6sc5c3n4-c3ccccc3)n2)cc1 ; c1ccc(-c2ccc(-c3nc(-c4ccccc4)nc(-c4cccc5c4c4ccc6c7cccc(-n8c9ccccc9c9ccccc98)c7sc6c4n5-c4ccccc4)n3)cc2)cc1 ; c1ccc(-c2cccc(-c3nc(-c4ccccc4)nc(-c4cccc5c4c4ccc6c7cccc(-n8c9ccccc9c9ccccc98)c7sc6c4n5-c4ccccc4)n3)c2)cc1 ; c1ccc(-c2cccc(-c3ccc(-c4nc(-c5ccccc5)nc(-c5cccc6c5c5ccc7c8cccc(-n9c%10ccccc%10c%10ccccc%109)c8sc7c5n6-c5ccccc5)n4)cc3)c2)cc1]

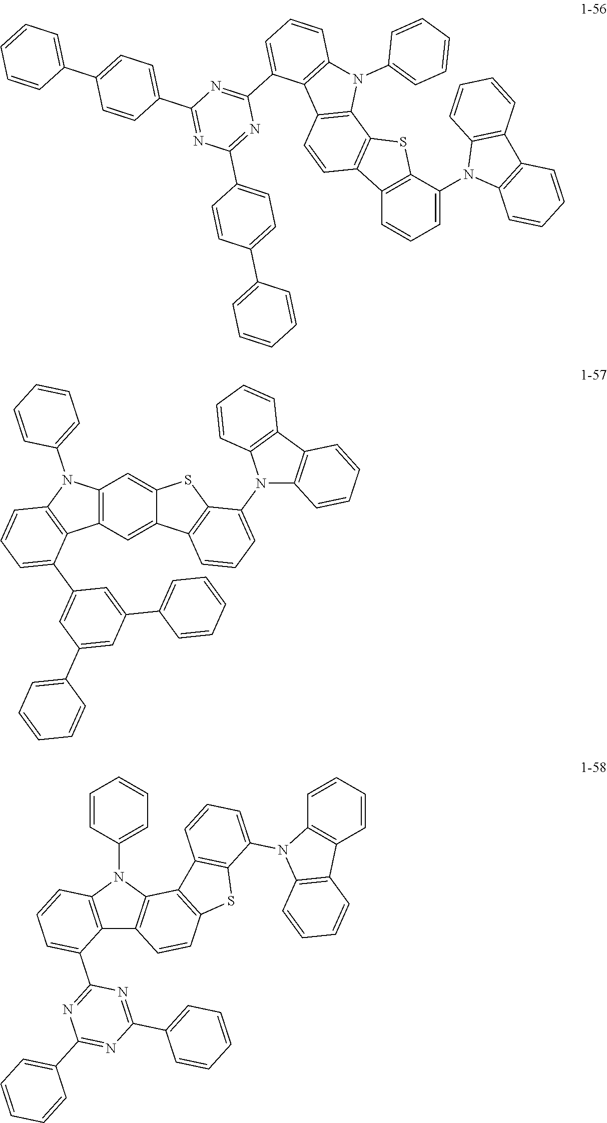 [SMILES: c1ccc(-c2ccc(-c3nc(-c4ccc(-c5ccccc5)cc4)nc(-c4cccc5c4c4ccc6c7cccc(-n8c9ccccc9c9ccccc98)c7sc6c4n5-c4ccccc4)n3)cc2)cc1 ; c1ccc(-c2cc(-c3ccccc3)cc(-c3cccc4c3c3cc5c(cc3n4-c3ccccc3)sc3c(-n4c6ccccc6c6ccccc64)cccc35)c2)cc1 ; c1ccc(-c2nc(-c3ccccc3)nc(-c3cccc4c3c3ccc5sc6c(-n7c8ccccc8c8ccccc87)cccc6c5c3n4-c3ccccc3)n2)cc1]

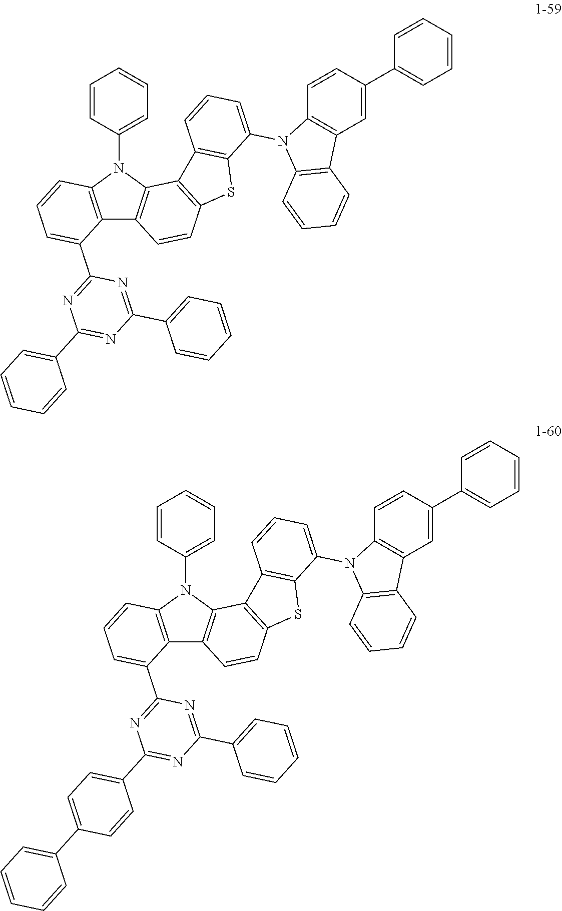 [SMILES: c1ccc(-c2ccc3c(c2)c2ccccc2n3-c2cccc3c2sc2ccc4c5c(-c6nc(-c7ccccc7)nc(-c7ccccc7)n6)cccc5n(-c5ccccc5)c4c23)cc1 ; c1ccc(-c2ccc(-c3nc(-c4ccccc4)nc(-c4cccc5c4c4ccc6sc7c(-n8c9ccccc9c9cc(-c%10ccccc%10)ccc98)cccc7c6c4n5-c4ccccc4)n3)cc2)cc1]

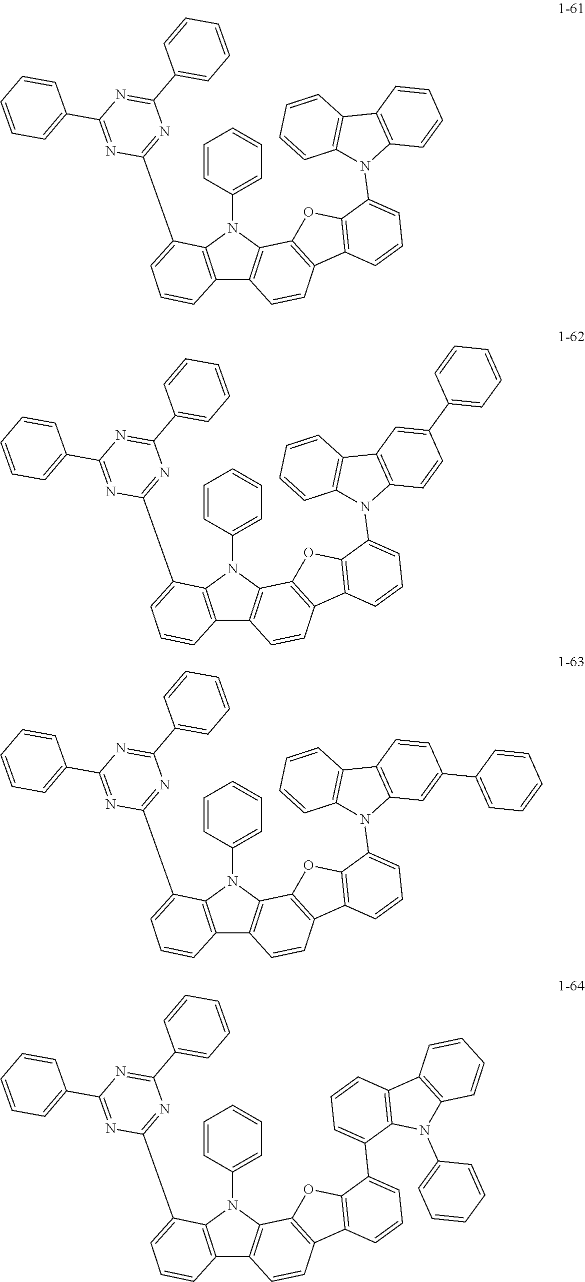

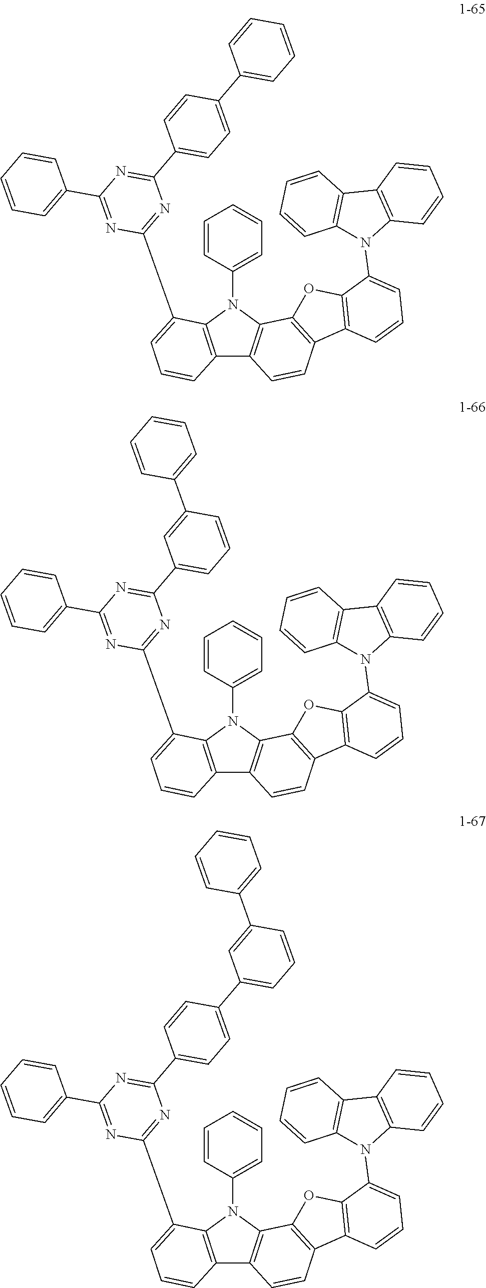

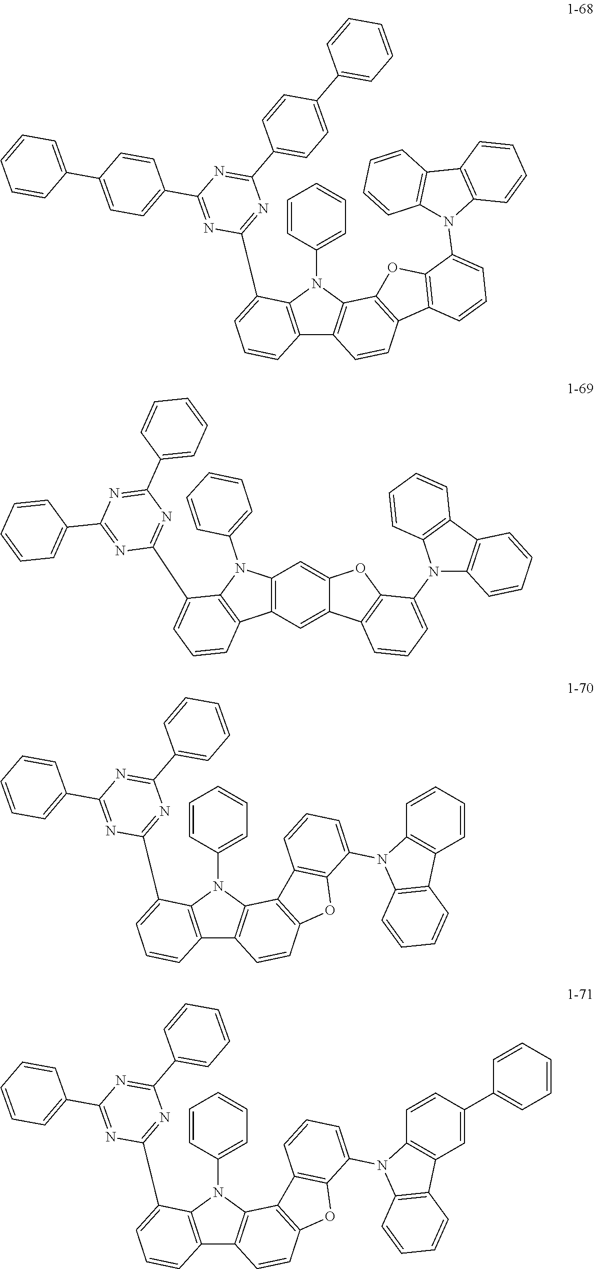

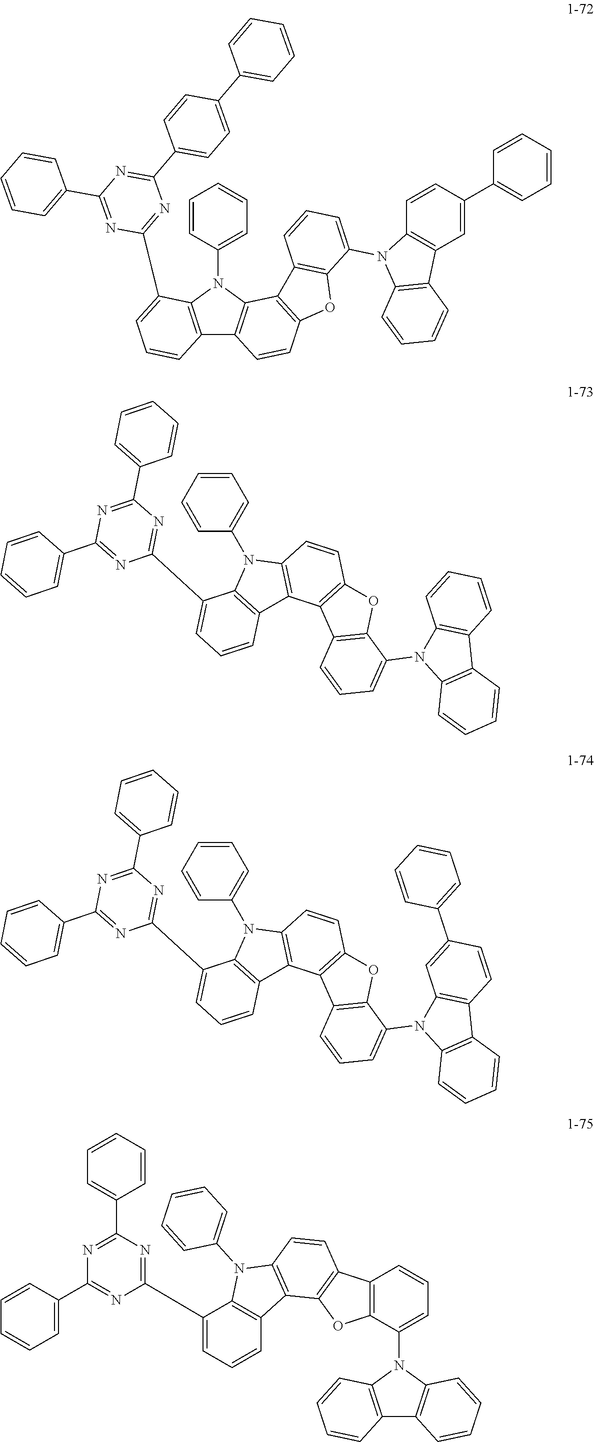 [SMILES: c1ccc(-c2ccc(-c3nc(-c4ccccc4)nc(-c4cccc5c6ccc7oc8c(-n9c%10ccccc%10c%10cc(-c%11ccccc%11)ccc%109)cccc8c7c6n(-c6ccccc6)c45)n3)cc2)cc1 ; c1ccc(-c2nc(-c3ccccc3)nc(-c3cccc4c5c6c(ccc5n(-c5ccccc5)c34)oc3c(-n4c5ccccc5c5ccccc54)cccc36)n2)cc1 ; c1ccc(-c2ccc3c4ccccc4n(-c4cccc5c4oc4ccc6c(c7cccc(-c8nc(-c9ccccc9)nc(-c9ccccc9)n8)c7n6-c6ccccc6)c45)c3c2)cc1 ; c1ccc(-c2nc(-c3ccccc3)nc(-c3cccc4c5c6oc7c(-n8c9ccccc9c9ccccc98)cccc7c6ccc5n(-c5ccccc5)c34)n2)cc1]

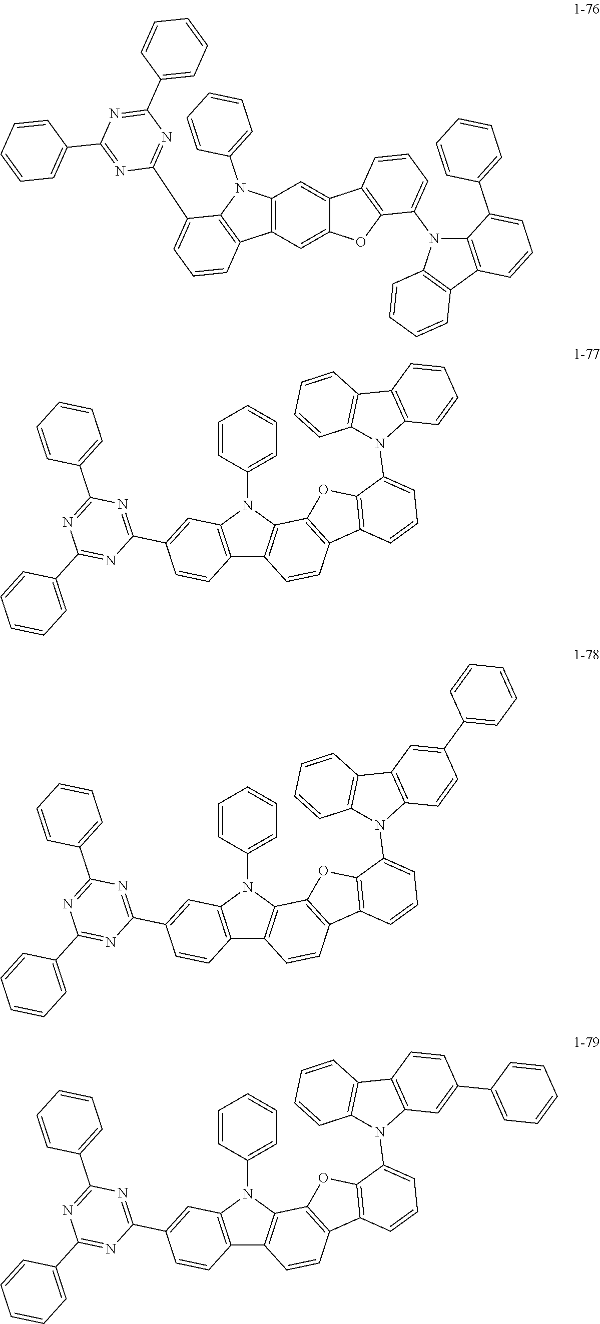

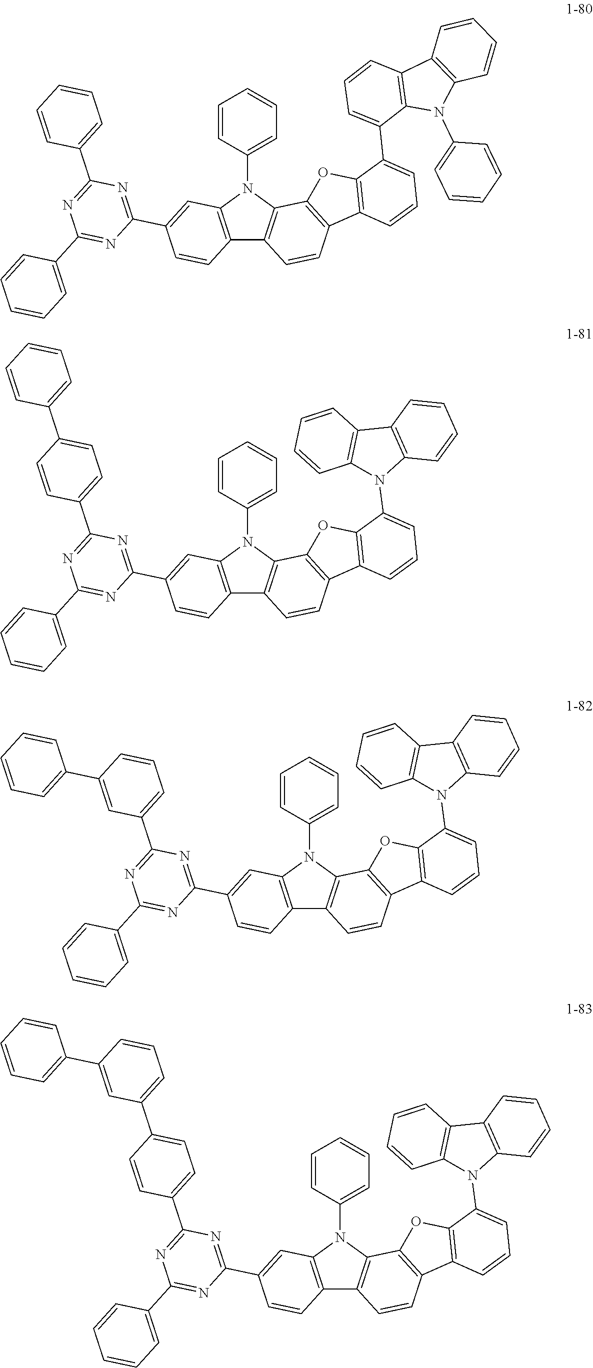

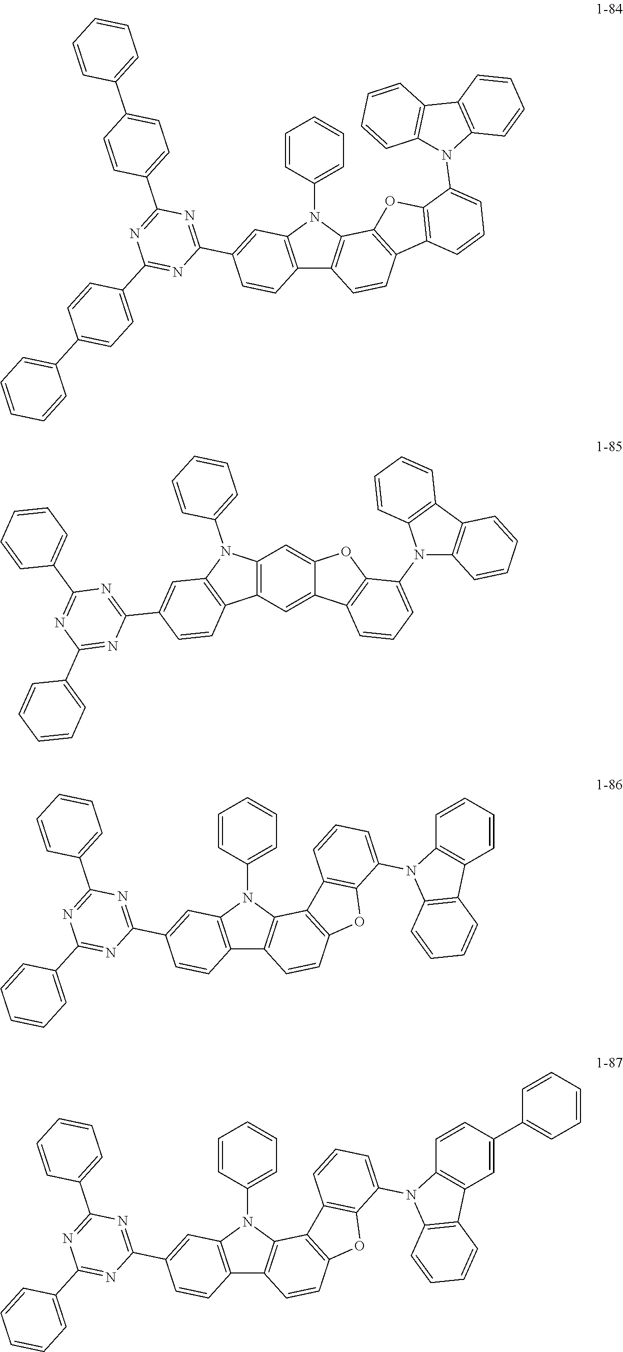 [SMILES: c1ccc(-c2ccc(-c3nc(-c4ccc(-c5ccccc5)cc4)nc(-c4ccc5c6ccc7c8cccc(-n9c%10ccccc%10c%10ccccc%109)c8oc7c6n(-c6ccccc6)c5c4)n3)cc2)cc1 ; c1ccc(-c2nc(-c3ccccc3)nc(-c3ccc4c5cc6c(cc5n(-c5ccccc5)c4c3)oc3c(-n4c5ccccc5c5ccccc54)cccc36)n2)cc1 ; c1ccc(-c2nc(-c3ccccc3)nc(-c3ccc4c5ccc6oc7c(-n8c9ccccc9c9ccccc98)cccc7c6c5n(-c5ccccc5)c4c3)n2)cc1 ; c1ccc(-c2ccc3c(c2)c2ccccc2n3-c2cccc3c2oc2ccc4c5ccc(-c6nc(-c7ccccc7)nc(-c7ccccc7)n6)cc5n(-c5ccccc5)c4c23)cc1]

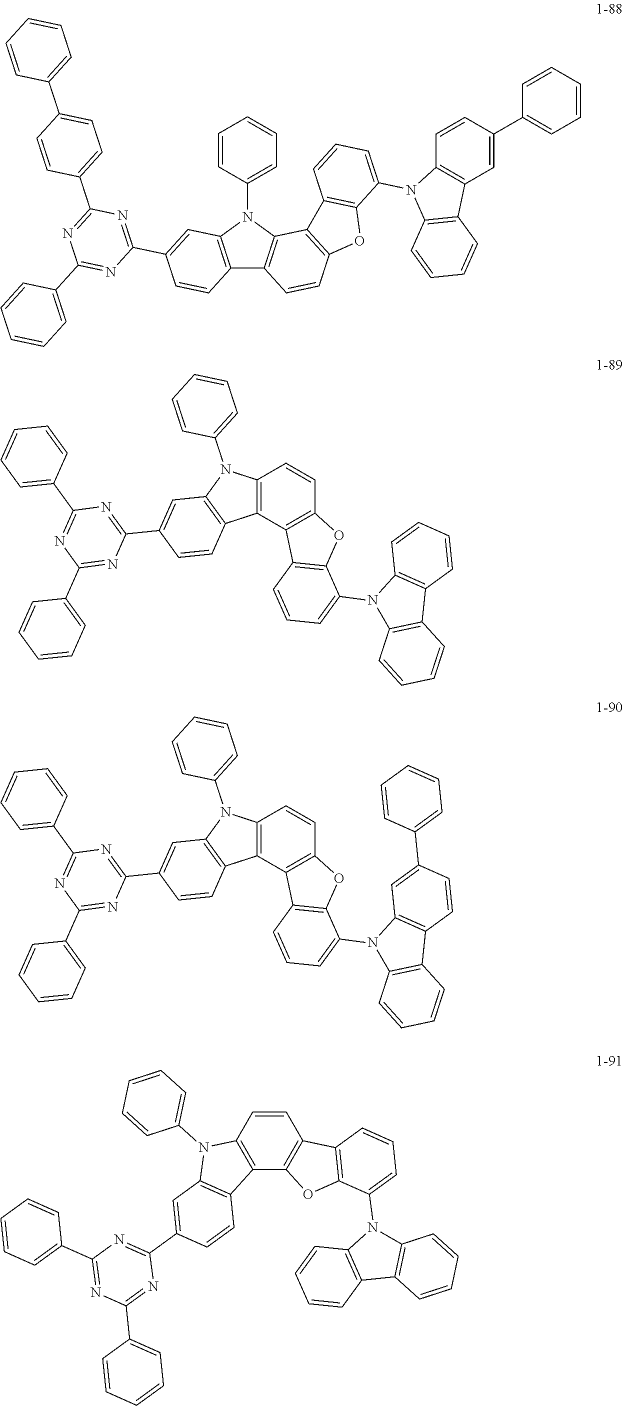 [SMILES: c1ccc(-c2ccc(-c3nc(-c4ccccc4)nc(-c4ccc5c6ccc7oc8c(-n9c%10ccccc%10c%10cc(-c%11ccccc%11)ccc%109)cccc8c7c6n(-c6ccccc6)c5c4)n3)cc2)cc1 ; c1ccc(-c2nc(-c3ccccc3)nc(-c3ccc4c5c6c(ccc5n(-c5ccccc5)c4c3)oc3c(-n4c5ccccc5c5ccccc54)cccc36)n2)cc1 ; c1ccc(-c2ccc3c4ccccc4n(-c4cccc5c4oc4ccc6c(c7ccc(-c8nc(-c9ccccc9)nc(-c9ccccc9)n8)cc7n6-c6ccccc6)c45)c3c2)cc1 ; c1ccc(-c2nc(-c3ccccc3)nc(-c3ccc4c5c6oc7c(-n8c9ccccc9c9ccccc98)cccc7c6ccc5n(-c5ccccc5)c4c3)n2)cc1]

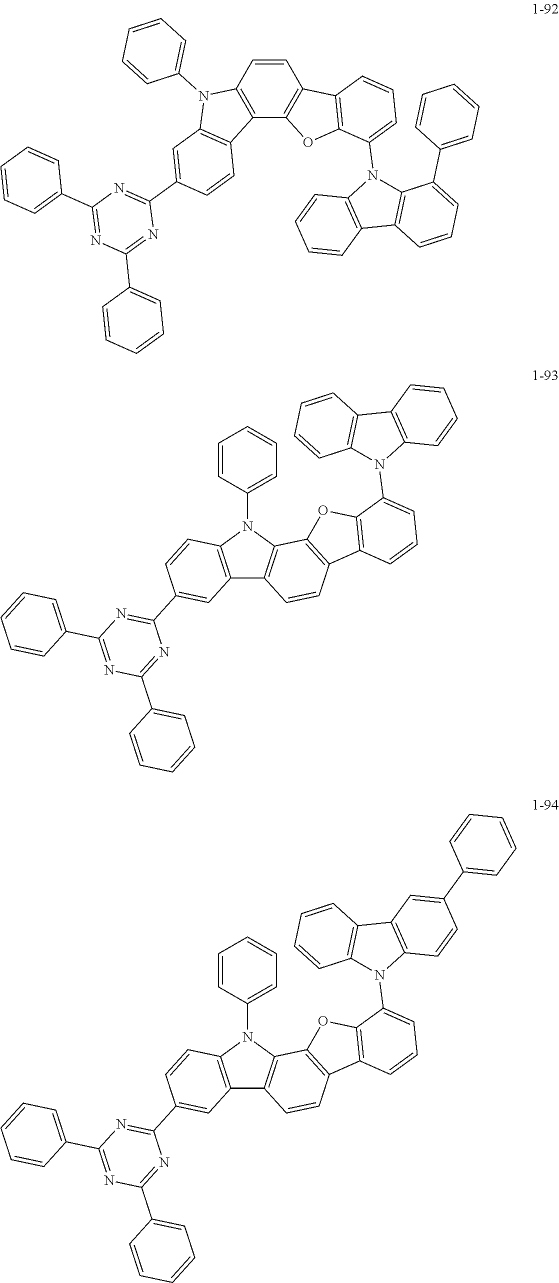 [SMILES: c1ccc(-c2nc(-c3ccccc3)nc(-c3ccc4c5c6oc7c(-n8c9ccccc9c9cccc(-c%10ccccc%10)c98)cccc7c6ccc5n(-c5ccccc5)c4c3)n2)cc1 ; c1ccc(-c2nc(-c3ccccc3)nc(-c3ccc4c(c3)c3ccc5c6cccc(-n7c8ccccc8c8ccccc87)c6oc5c3n4-c3ccccc3)n2)cc1 ; c1ccc(-c2ccc3c(c2)c2ccccc2n3-c2cccc3c2oc2c3ccc3c4cc(-c5nc(-c6ccccc6)nc(-c6ccccc6)n5)ccc4n(-c4ccccc4)c32)cc1]

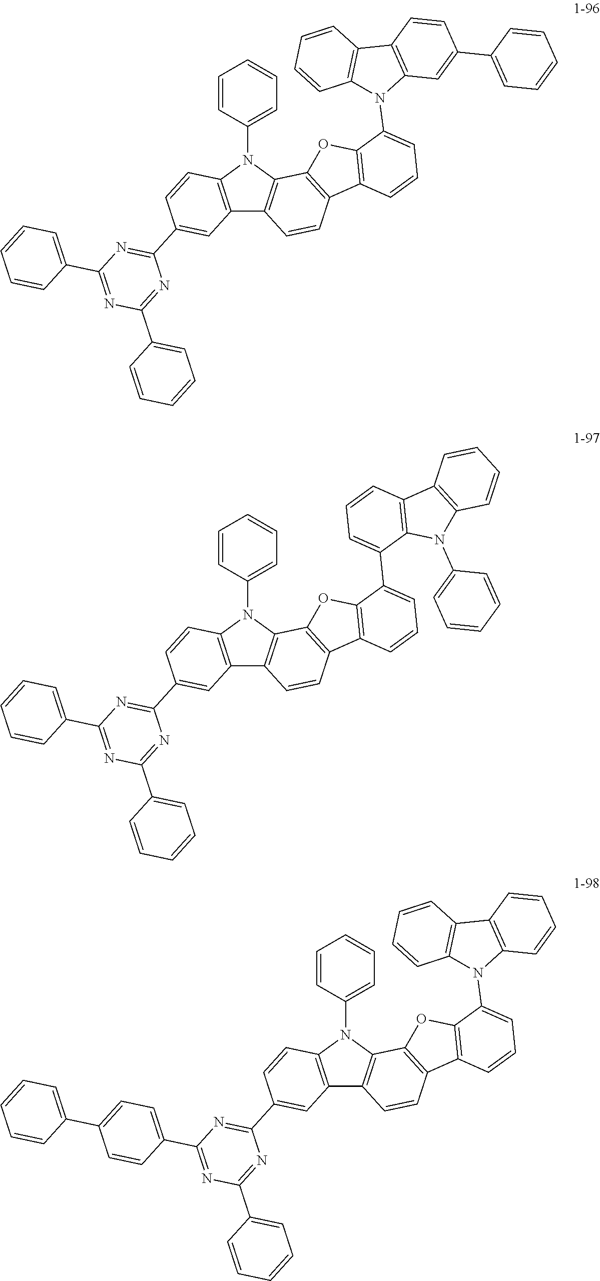 [SMILES: c1ccc(-c2ccc3c4ccccc4n(-c4cccc5c4oc4c5ccc5c6cc(-c7nc(-c8ccccc8)nc(-c8ccccc8)n7)ccc6n(-c6ccccc6)c54)c3c2)cc1 ; c1ccc(-c2nc(-c3ccccc3)nc(-c3ccc4c(c3)c3ccc5c6cccc(-c7cccc8c9ccccc9n(-c9ccccc9)c78)c6oc5c3n4-c3ccccc3)n2)cc1 ; c1ccc(-c2ccc(-c3nc(-c4ccccc4)nc(-c4ccc5c(c4)c4ccc6c7cccc(-n8c9ccccc9c9ccccc98)c7oc6c4n5-c4ccccc4)n3)cc2)cc1]

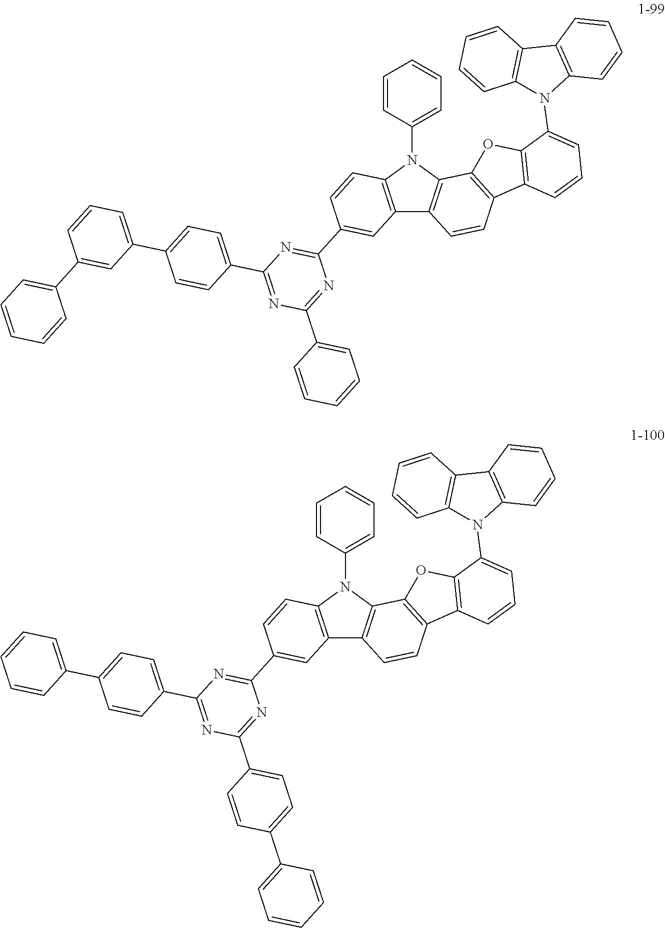 [SMILES: c1ccc(-c2cccc(-c3ccc(-c4nc(-c5ccccc5)nc(-c5ccc6c(c5)c5ccc7c8cccc(-n9c%10ccccc%10c%10ccccc%109)c8oc7c5n6-c5ccccc5)n4)cc3)c2)cc1 ; c1ccc(-c2ccc(-c3nc(-c4ccc(-c5ccccc5)cc4)nc(-c4ccc5c(c4)c4ccc6c7cccc(-n8c9ccccc9c9ccccc98)c7oc6c4n5-c4ccccc4)n3)cc2)cc1]

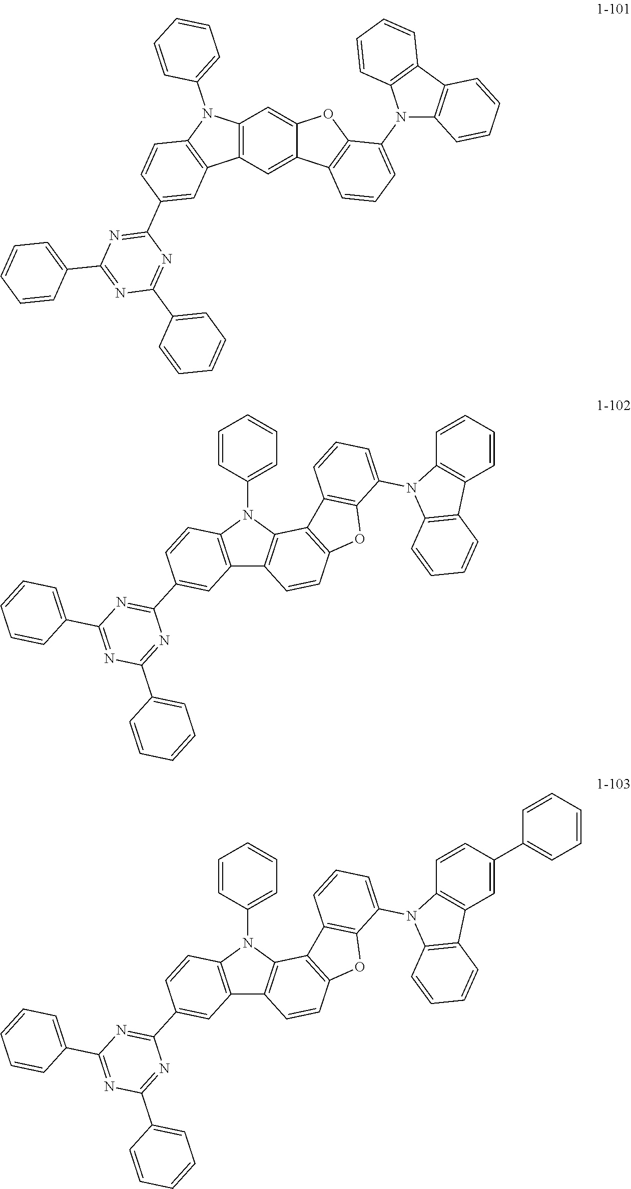 [SMILES: c1ccc(-c2nc(-c3ccccc3)nc(-c3ccc4c(c3)c3cc5c(cc3n4-c3ccccc3)oc3c(-n4c6ccccc6c6ccccc64)cccc35)n2)cc1 ; c1ccc(-c2nc(-c3ccccc3)nc(-c3ccc4c(c3)c3ccc5oc6c(-n7c8ccccc8c8ccccc87)cccc6c5c3n4-c3ccccc3)n2)cc1 ; c1ccc(-c2ccc3c(c2)c2ccccc2n3-c2cccc3c2oc2ccc4c5cc(-c6nc(-c7ccccc7)nc(-c7ccccc7)n6)ccc5n(-c5ccccc5)c4c23)cc1]

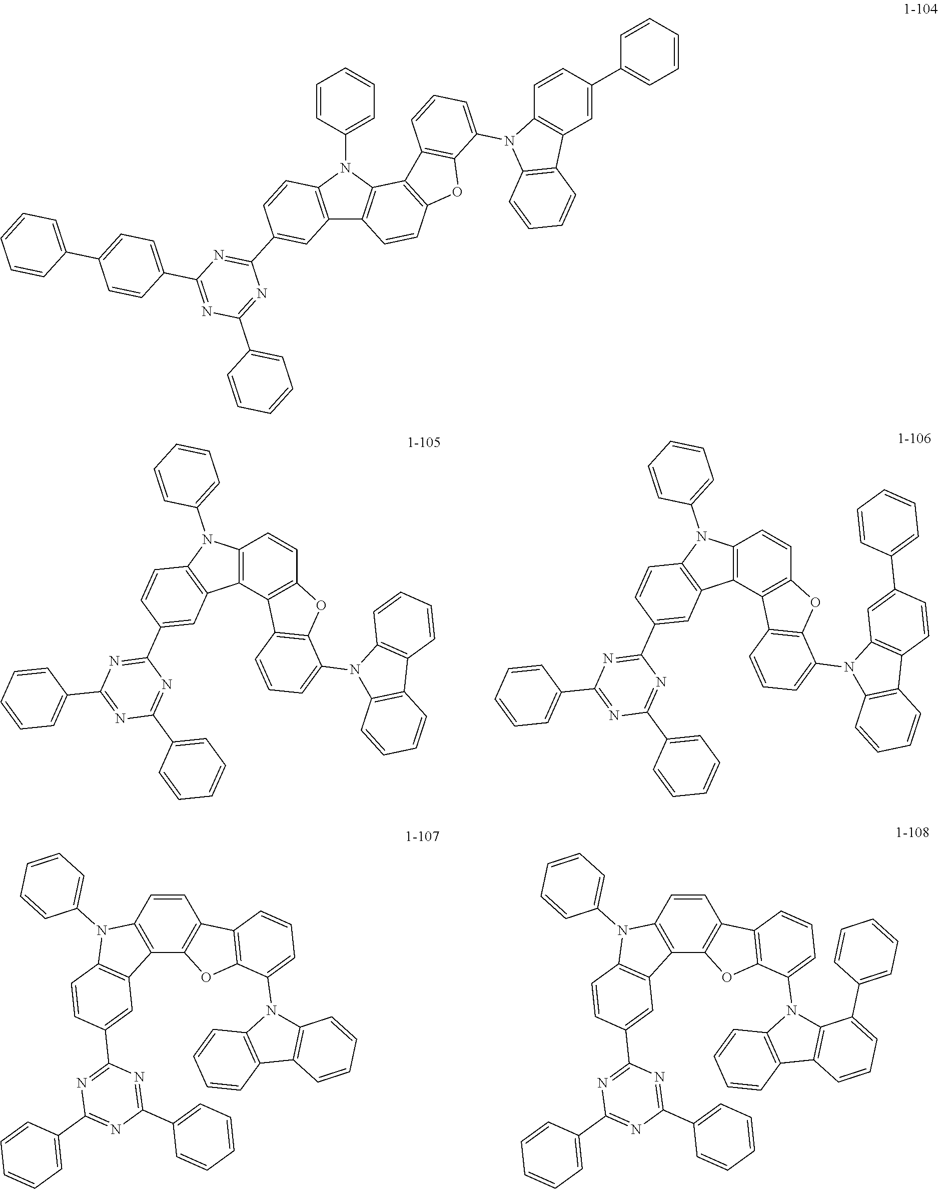

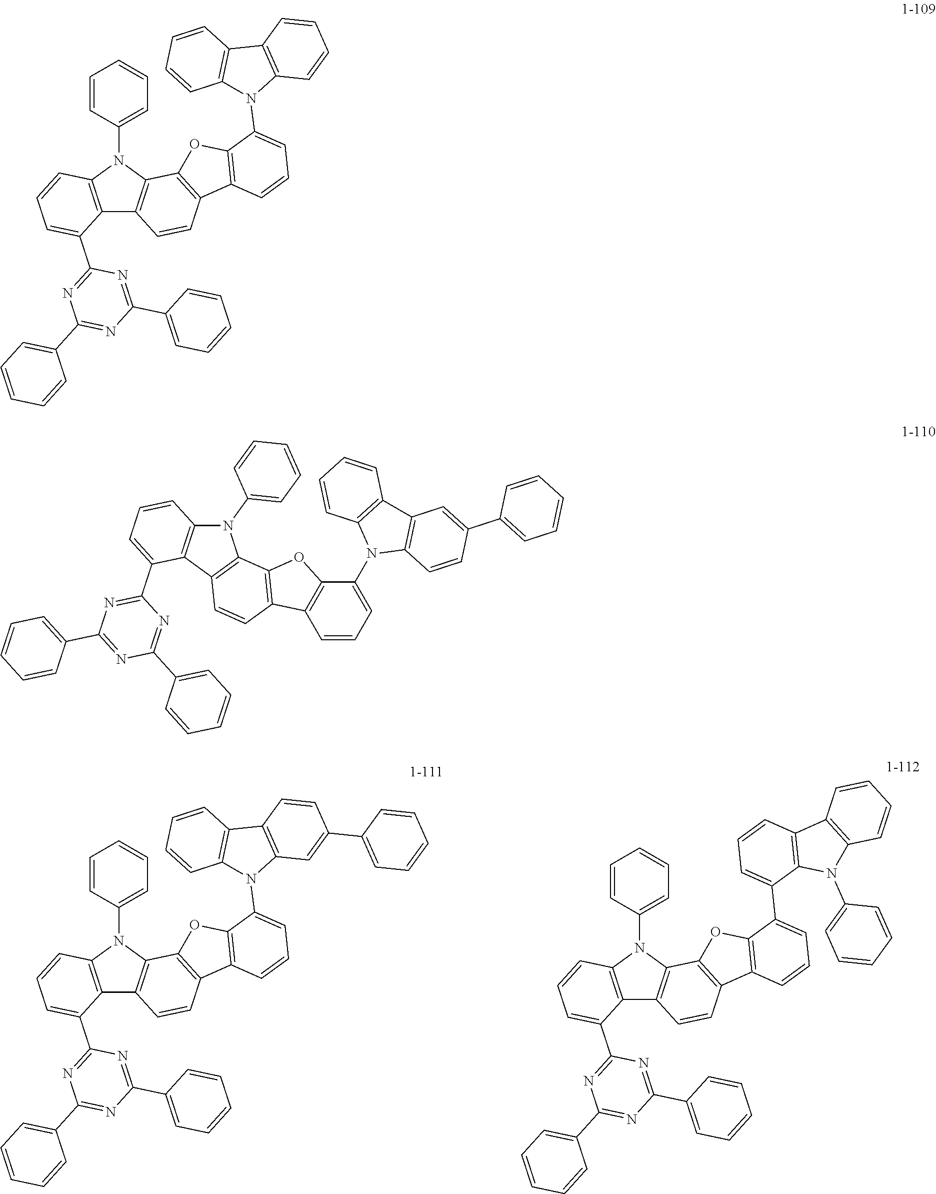 [SMILES: c1ccc(-c2nc(-c3ccccc3)nc(-c3cccc4c3c3ccc5c6cccc(-n7c8ccccc8c8ccccc87)c6oc5c3n4-c3ccccc3)n2)cc1 ; c1ccc(-c2ccc3c(c2)c2ccccc2n3-c2cccc3c2oc2c3ccc3c4c(-c5nc(-c6ccccc6)nc(-c6ccccc6)n5)cccc4n(-c4ccccc4)c32)cc1 ; c1ccc(-c2ccc3c4ccccc4n(-c4cccc5c4oc4c5ccc5c6c(-c7nc(-c8ccccc8)nc(-c8ccccc8)n7)cccc6n(-c6ccccc6)c54)c3c2)cc1 ; c1ccc(-c2nc(-c3ccccc3)nc(-c3cccc4c3c3ccc5c6cccc(-c7cccc8c9ccccc9n(-c9ccccc9)c78)c6oc5c3n4-c3ccccc3)n2)cc1]

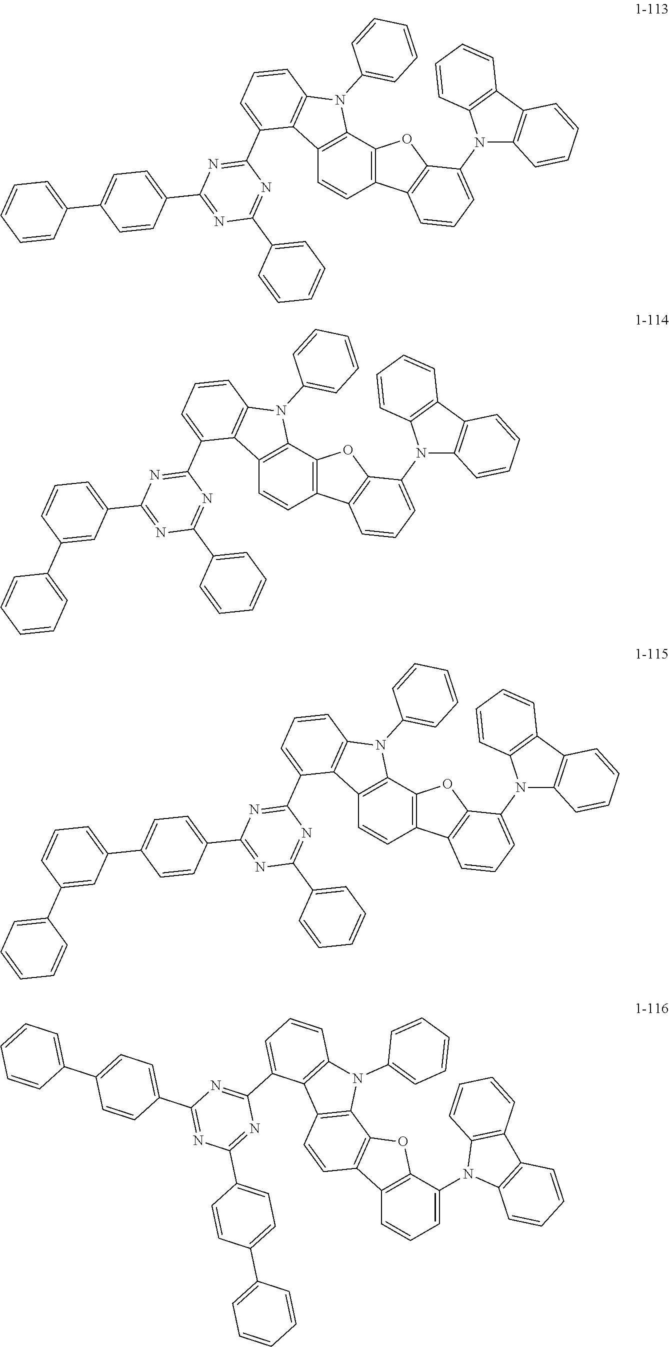

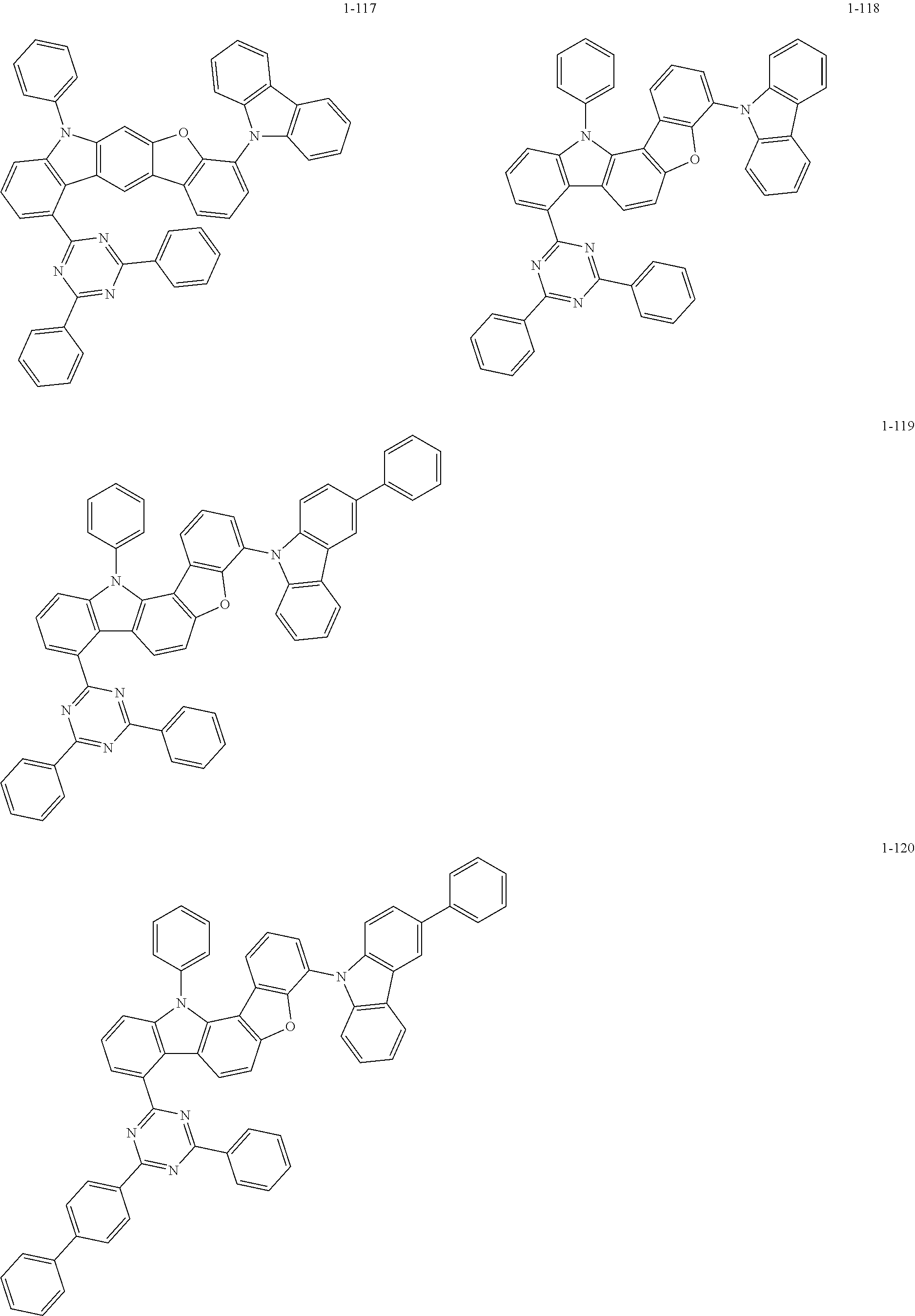 [SMILES: c1ccc(-c2nc(-c3ccccc3)nc(-c3cccc4c3c3cc5c(cc3n4-c3ccccc3)oc3c(-n4c6ccccc6c6ccccc64)cccc35)n2)cc1 ; c1ccc(-c2nc(-c3ccccc3)nc(-c3cccc4c3c3ccc5oc6c(-n7c8ccccc8c8ccccc87)cccc6c5c3n4-c3ccccc3)n2)cc1 ; c1ccc(-c2ccc3c(c2)c2ccccc2n3-c2cccc3c2oc2ccc4c5c(-c6nc(-c7ccccc7)nc(-c7ccccc7)n6)cccc5n(-c5ccccc5)c4c23)cc1 ; c1ccc(-c2ccc(-c3nc(-c4ccccc4)nc(-c4cccc5c4c4ccc6oc7c(-n8c9ccccc9c9cc(-c%10ccccc%10)ccc98)cccc7c6c4n5-c4ccccc4)n3)cc2)cc1]

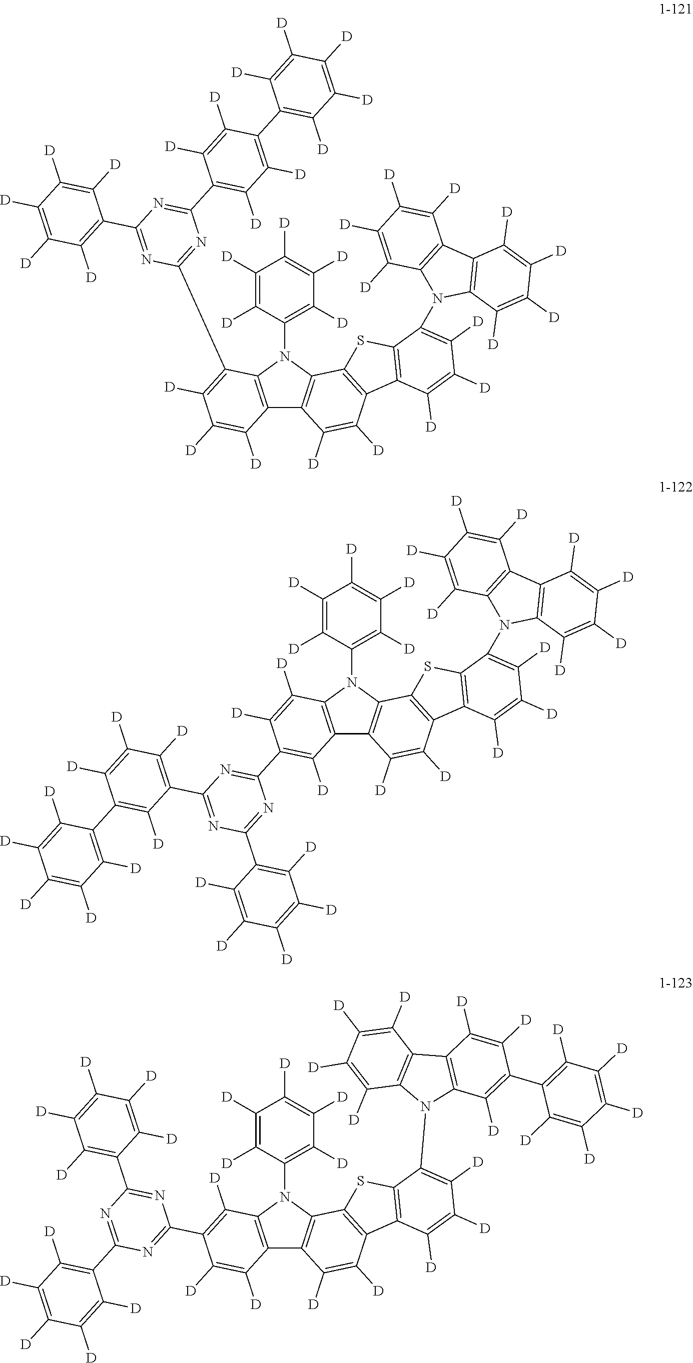

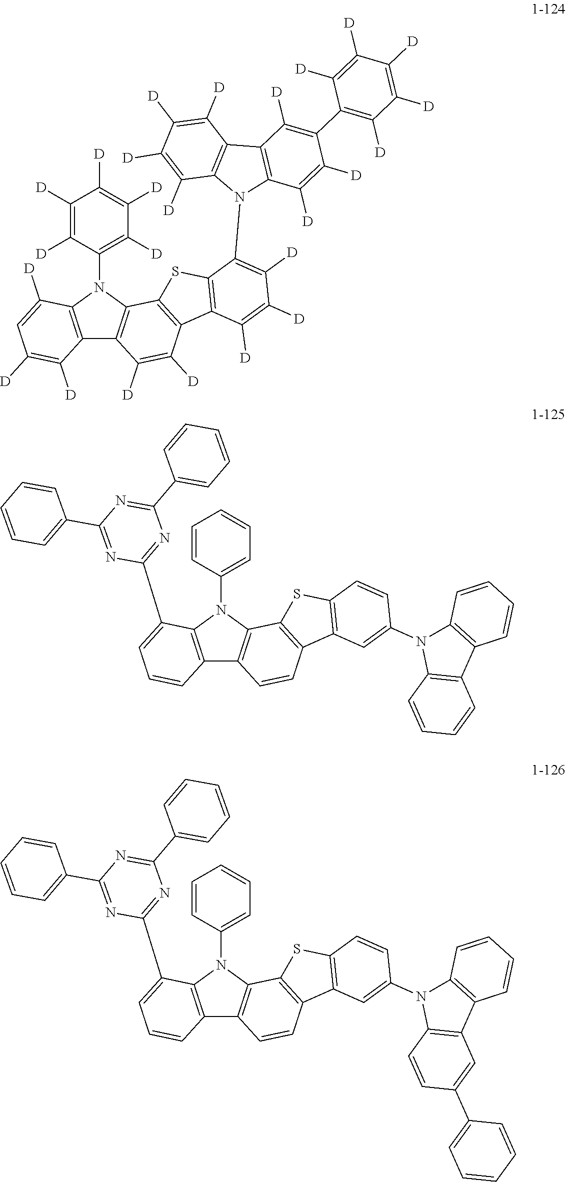

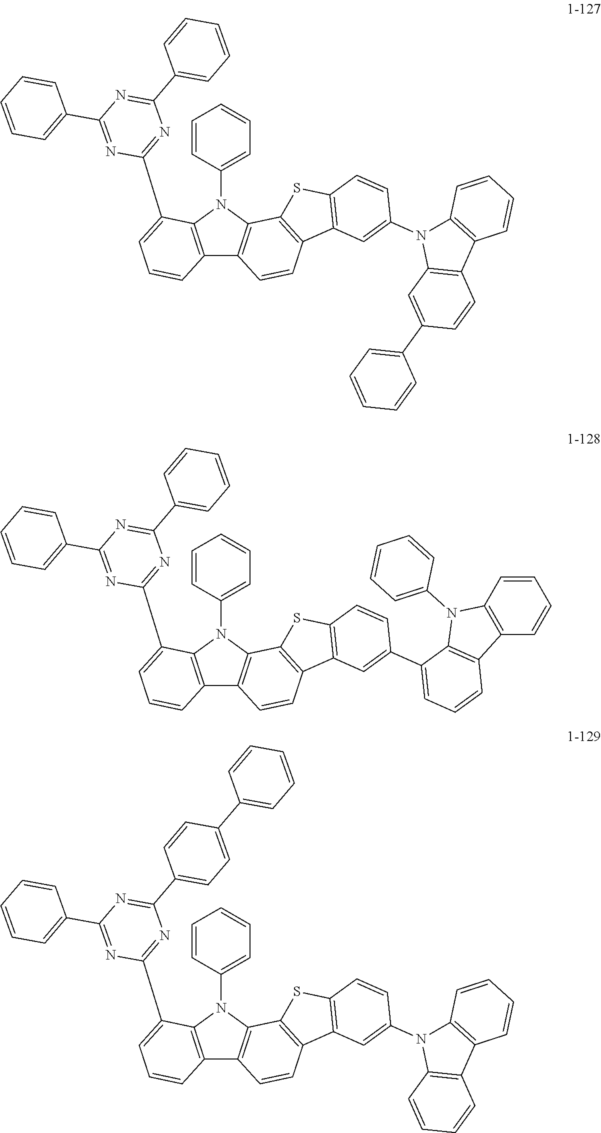

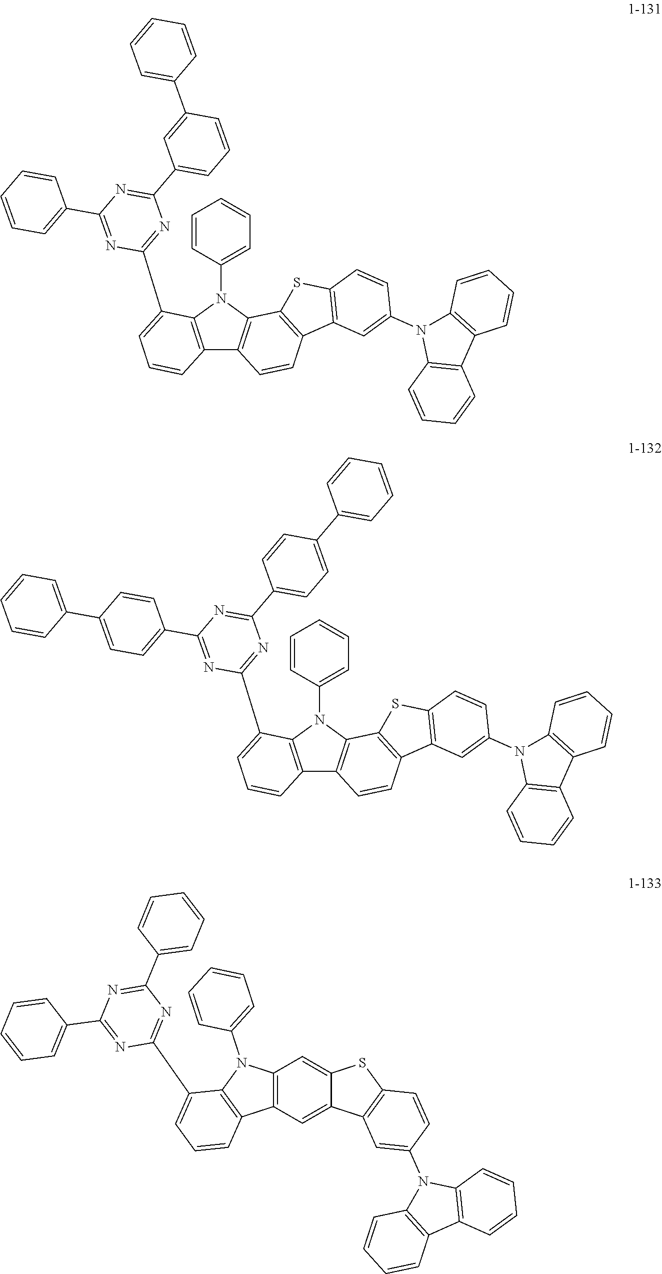 [SMILES: c1ccc(-c2cccc(-c3nc(-c4ccccc4)nc(-c4cccc5c6ccc7c8cc(-n9c%10ccccc%10c%10ccccc%109)ccc8sc7c6n(-c6ccccc6)c45)n3)c2)cc1 ; c1ccc(-c2ccc(-c3nc(-c4ccc(-c5ccccc5)cc4)nc(-c4cccc5c6ccc7c8cc(-n9c%10ccccc%10c%10ccccc%109)ccc8sc7c6n(-c6ccccc6)c45)n3)cc2)cc1 ; c1ccc(-c2nc(-c3ccccc3)nc(-c3cccc4c5cc6c(cc5n(-c5ccccc5)c34)sc3ccc(-n4c5ccccc5c5ccccc54)cc36)n2)cc1]

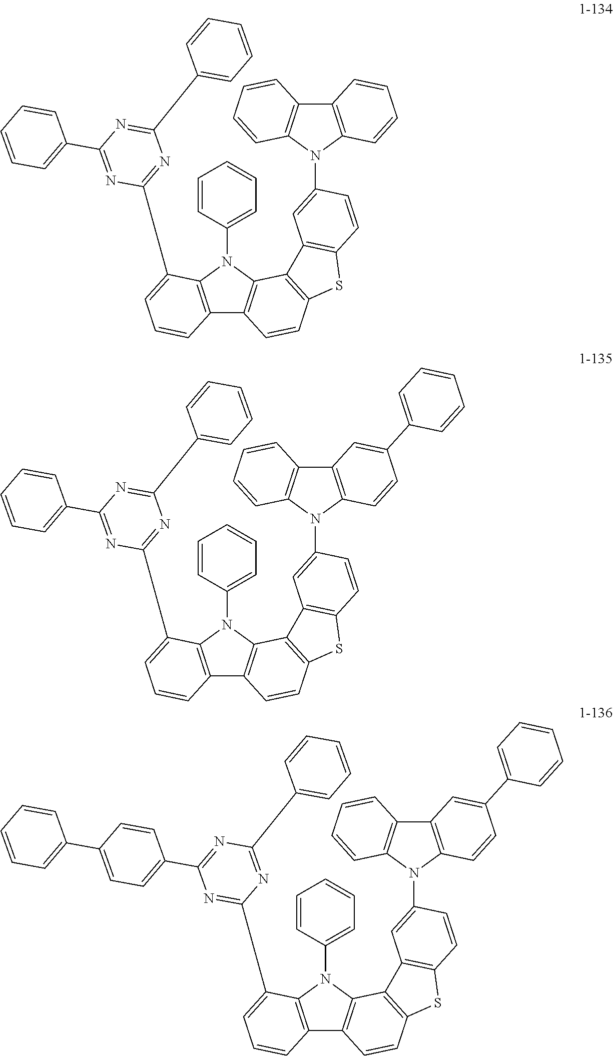

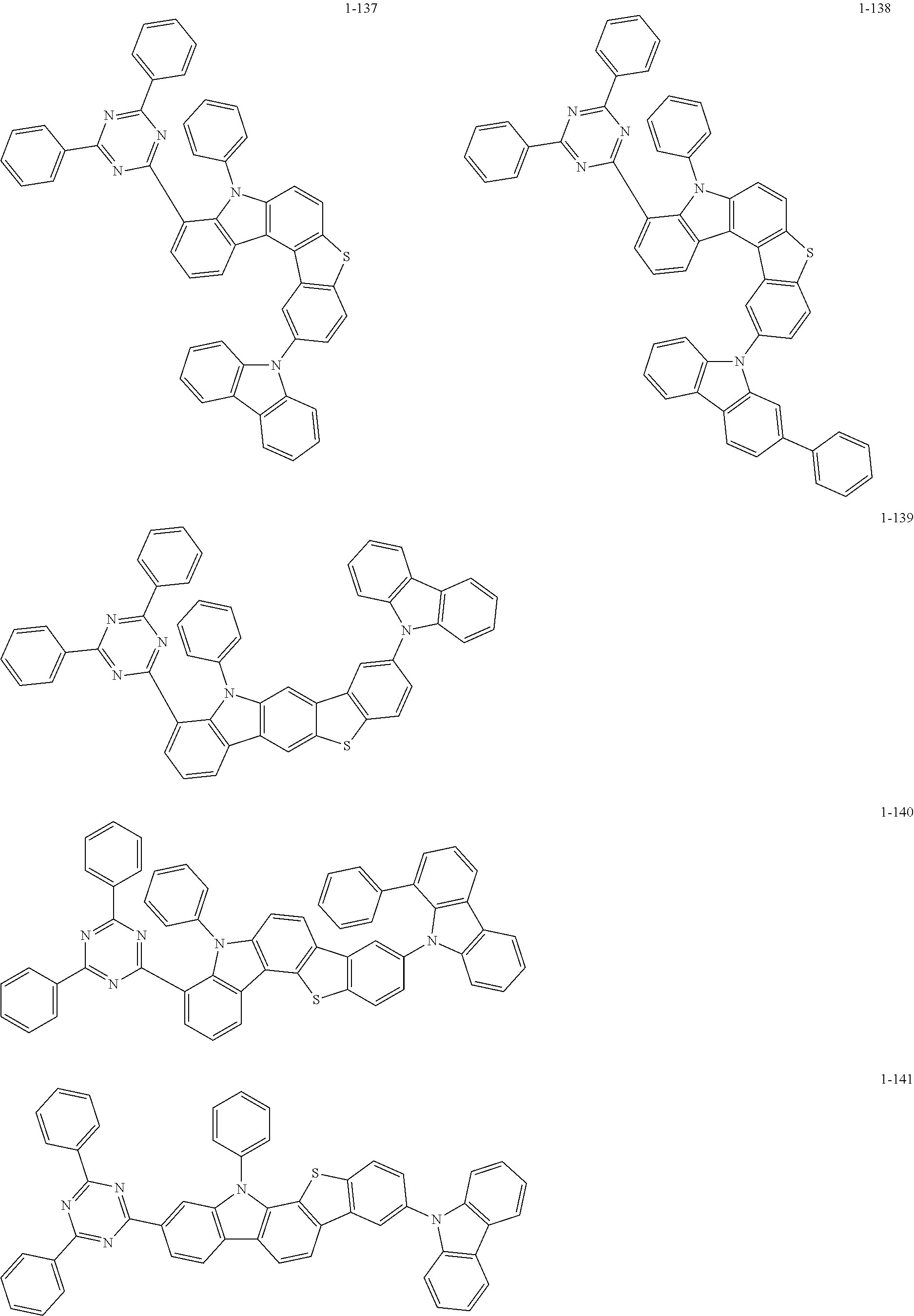

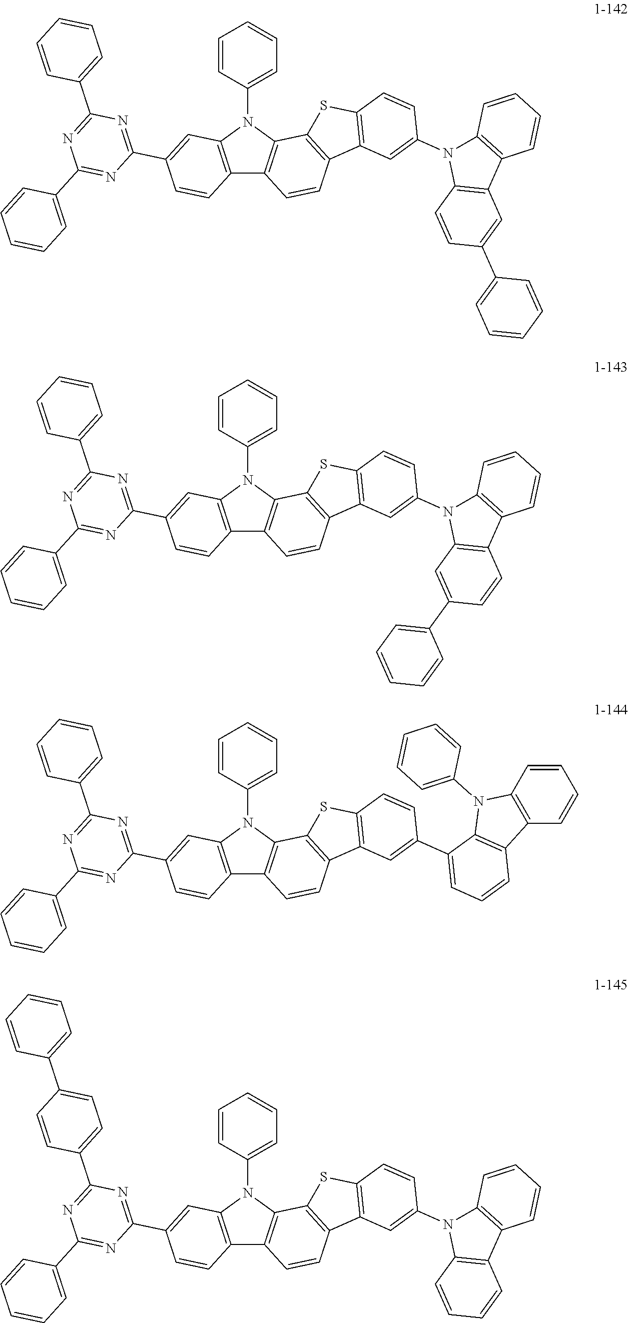

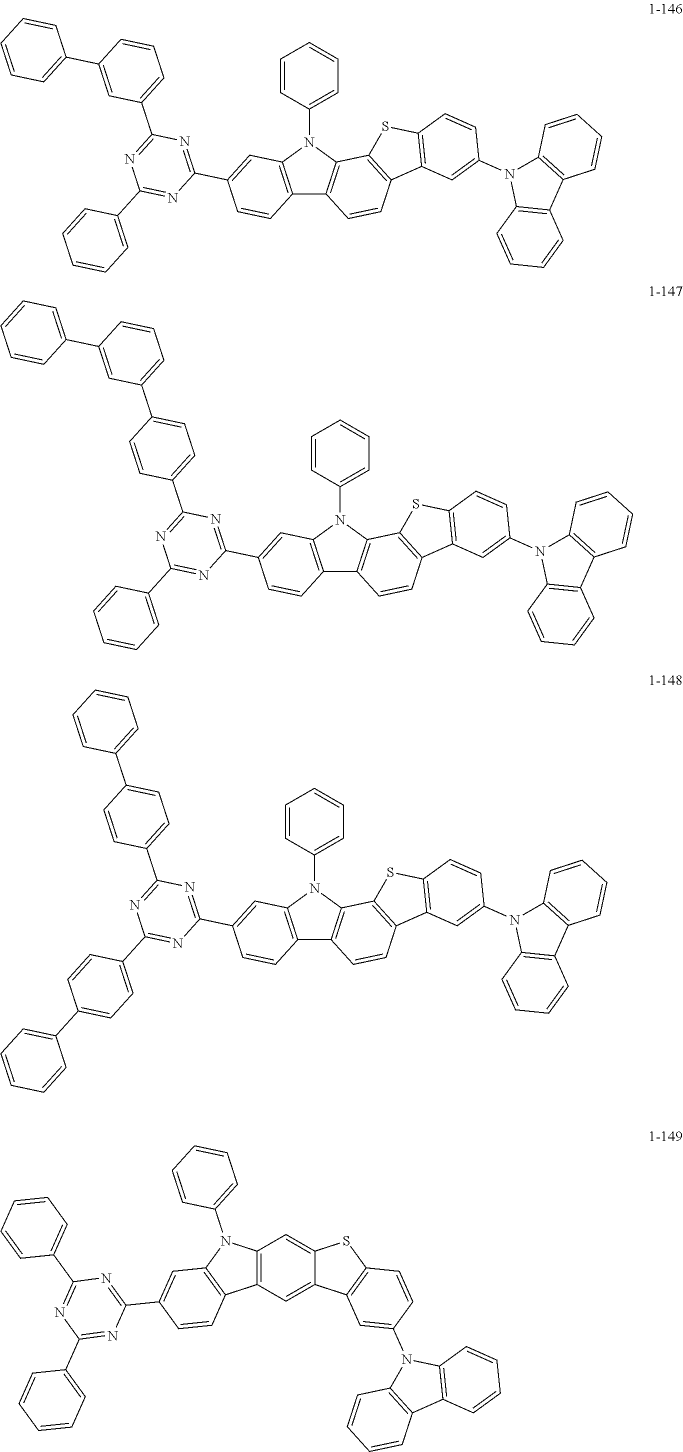

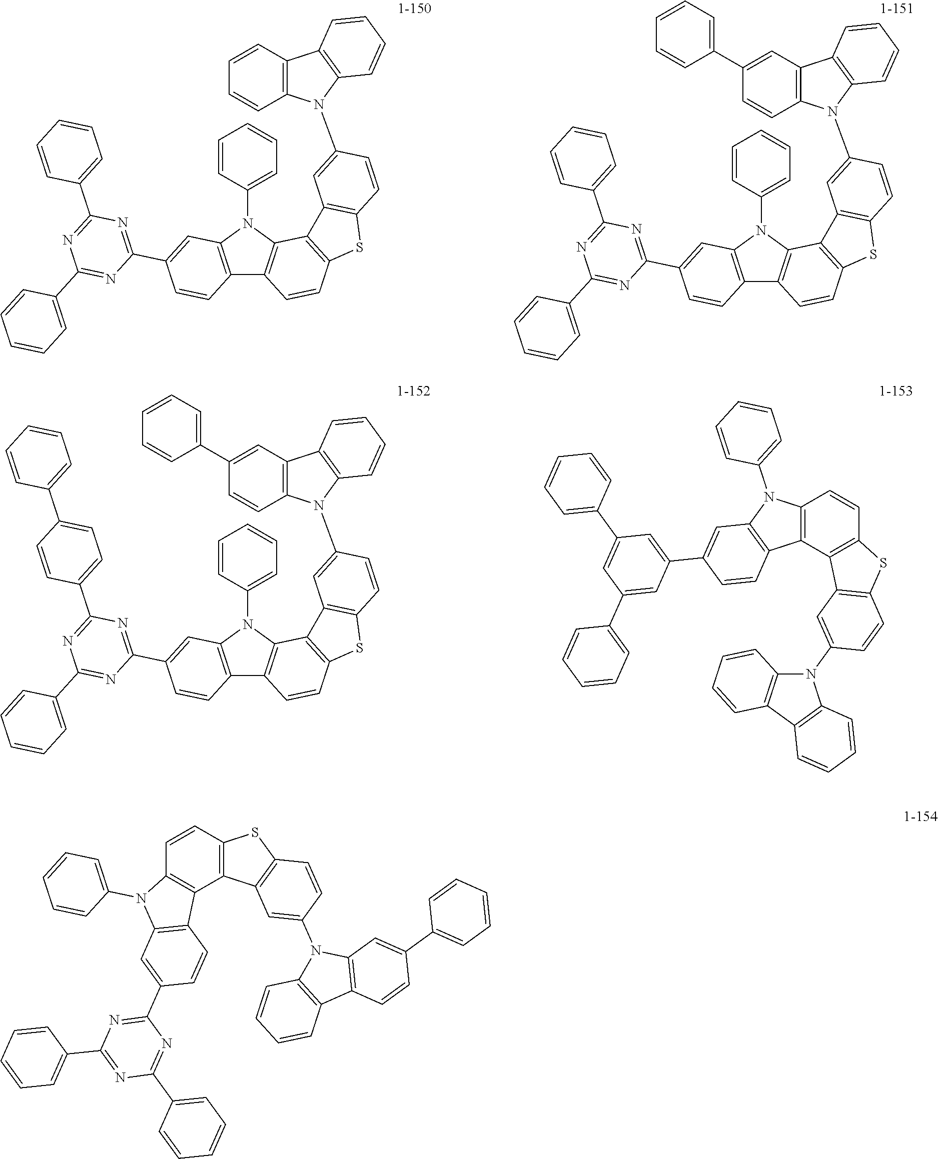

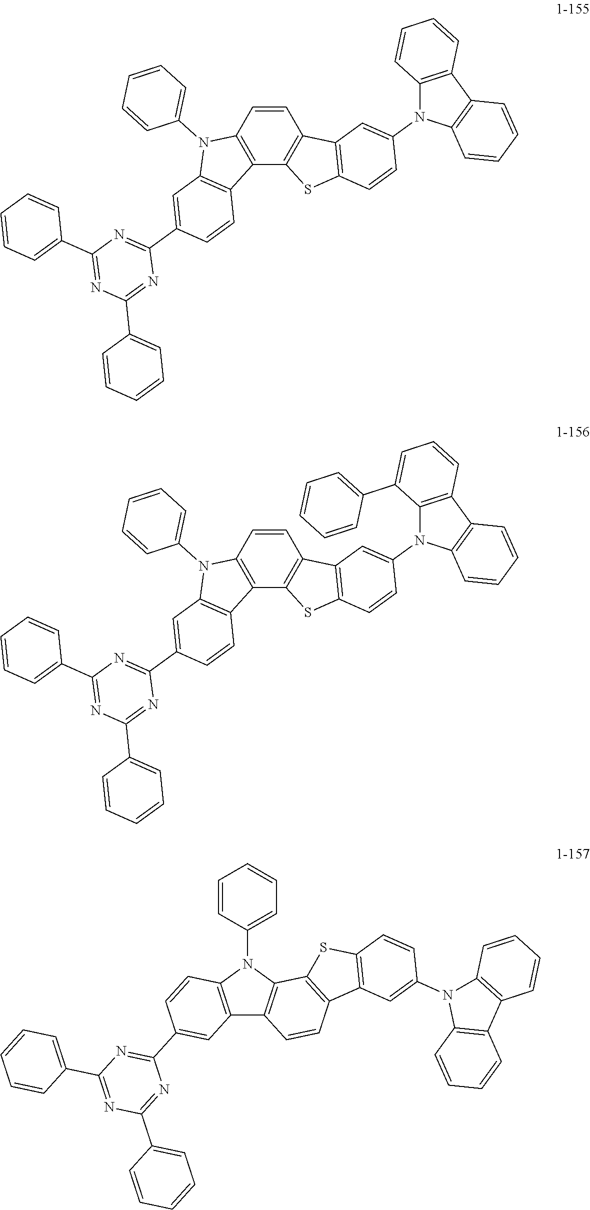 [SMILES: c1ccc(-c2nc(-c3ccccc3)nc(-c3ccc4c5c6sc7ccc(-n8c9ccccc9c9ccccc98)cc7c6ccc5n(-c5ccccc5)c4c3)n2)cc1 ; c1ccc(-c2nc(-c3ccccc3)nc(-c3ccc4c5c6sc7ccc(-n8c9ccccc9c9cccc(-c%10ccccc%10)c98)cc7c6ccc5n(-c5ccccc5)c4c3)n2)cc1 ; c1ccc(-c2nc(-c3ccccc3)nc(-c3ccc4c(c3)c3ccc5c6cc(-n7c8ccccc8c8ccccc87)ccc6sc5c3n4-c3ccccc3)n2)cc1]

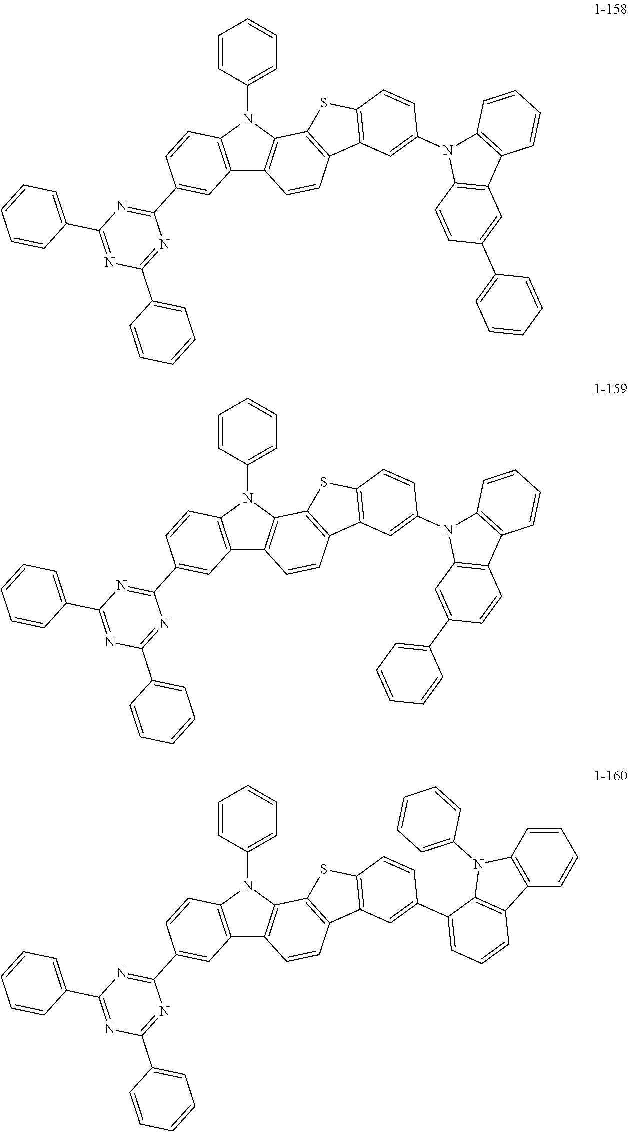 [SMILES: c1ccc(-c2ccc3c(c2)c2ccccc2n3-c2ccc3sc4c(ccc5c6cc(-c7nc(-c8ccccc8)nc(-c8ccccc8)n7)ccc6n(-c6ccccc6)c54)c3c2)cc1 ; c1ccc(-c2ccc3c4ccccc4n(-c4ccc5sc6c(ccc7c8cc(-c9nc(-c%10ccccc%10)nc(-c%10ccccc%10)n9)ccc8n(-c8ccccc8)c76)c5c4)c3c2)cc1 ; c1ccc(-c2nc(-c3ccccc3)nc(-c3ccc4c(c3)c3ccc5c6cc(-c7cccc8c9ccccc9n(-c9ccccc9)c78)ccc6sc5c3n4-c3ccccc3)n2)cc1]

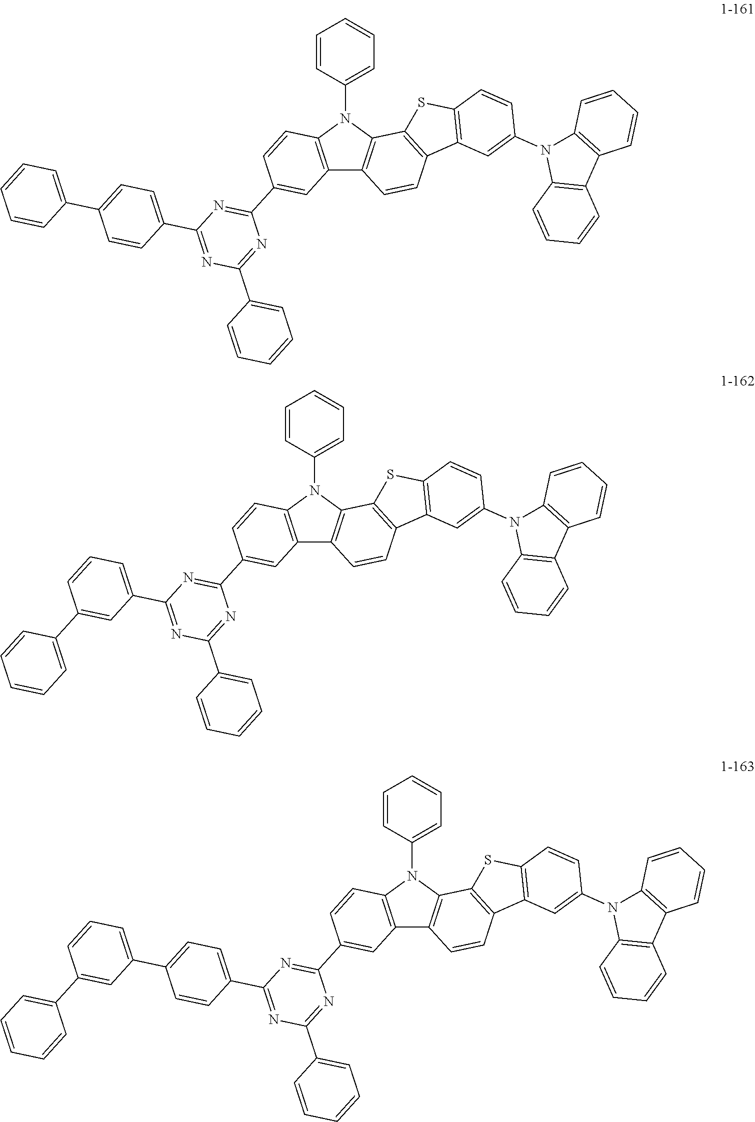

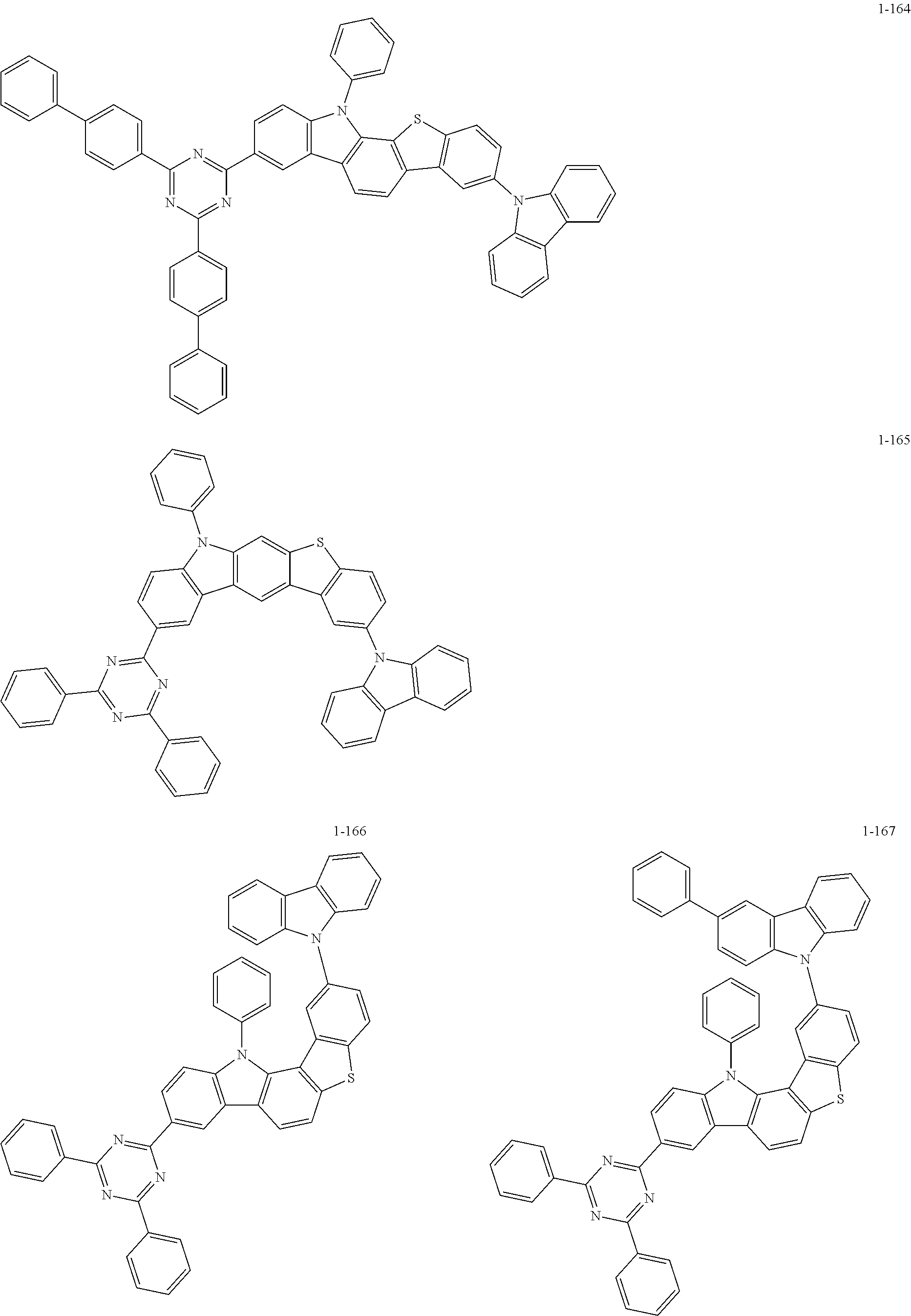

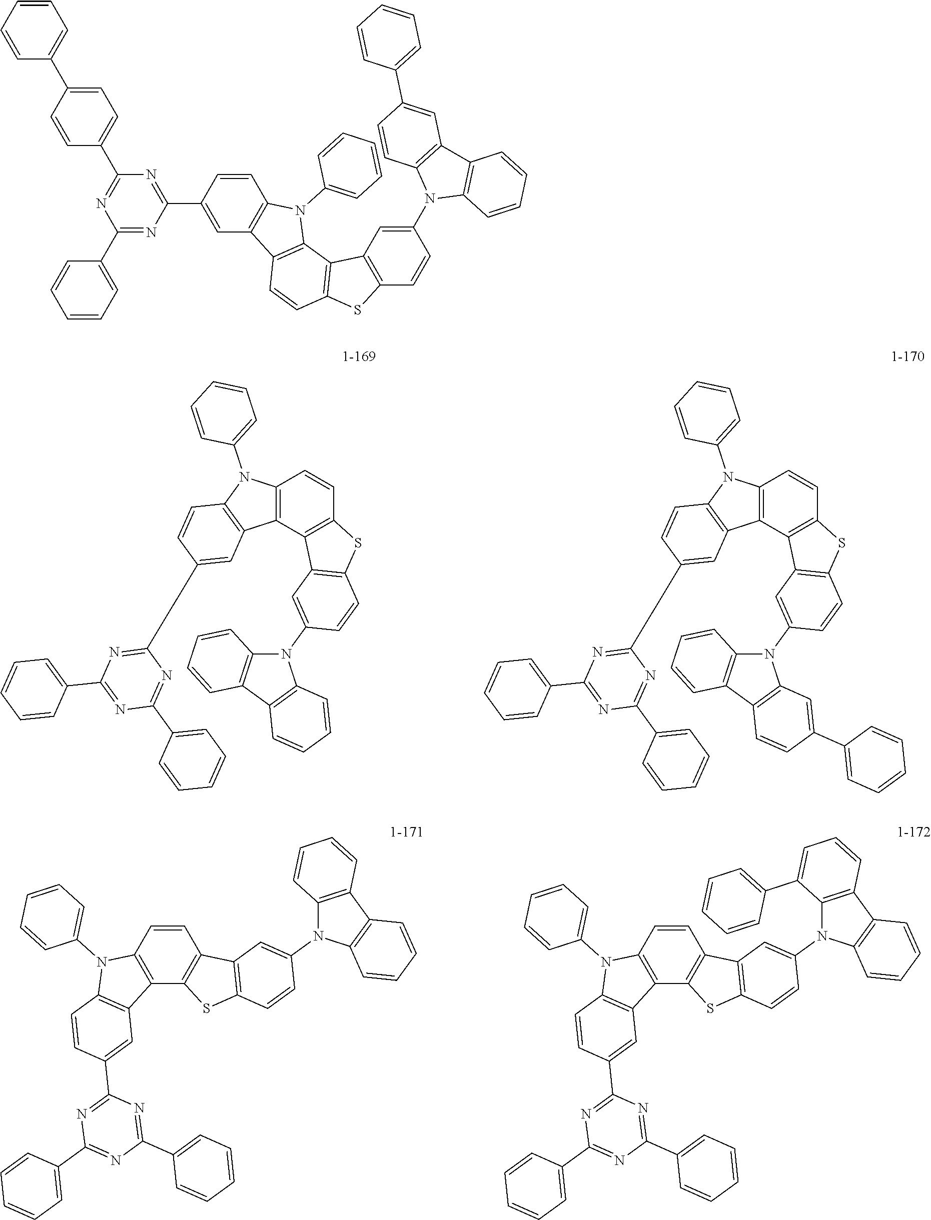

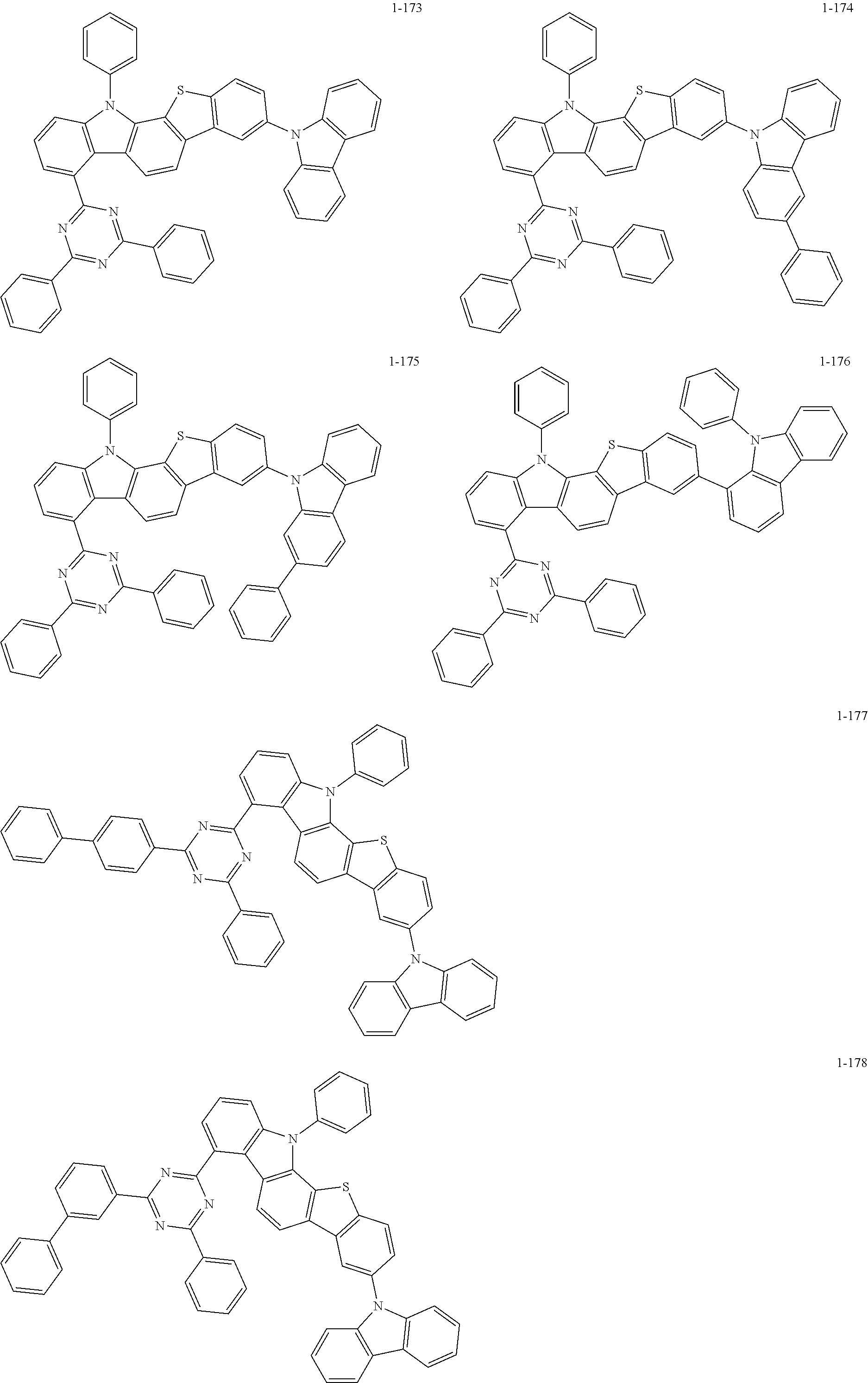

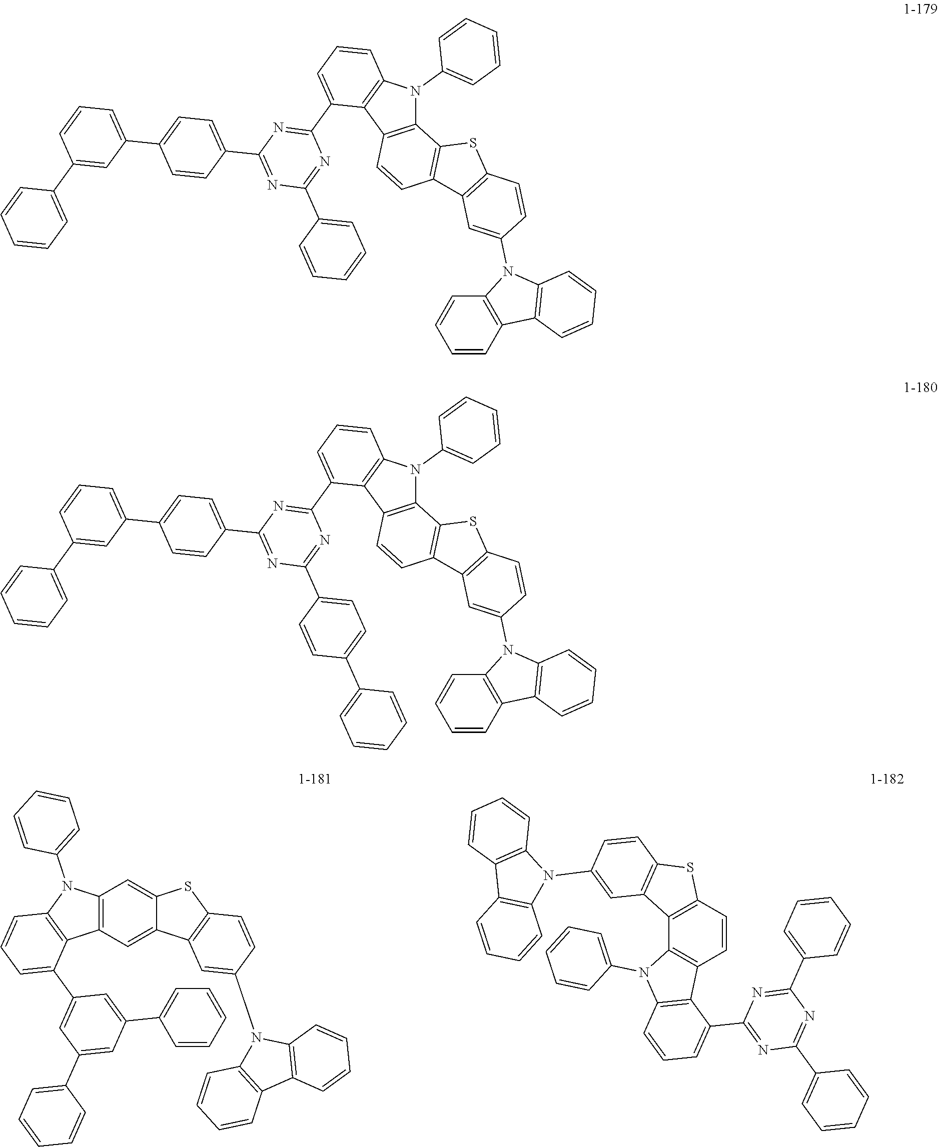

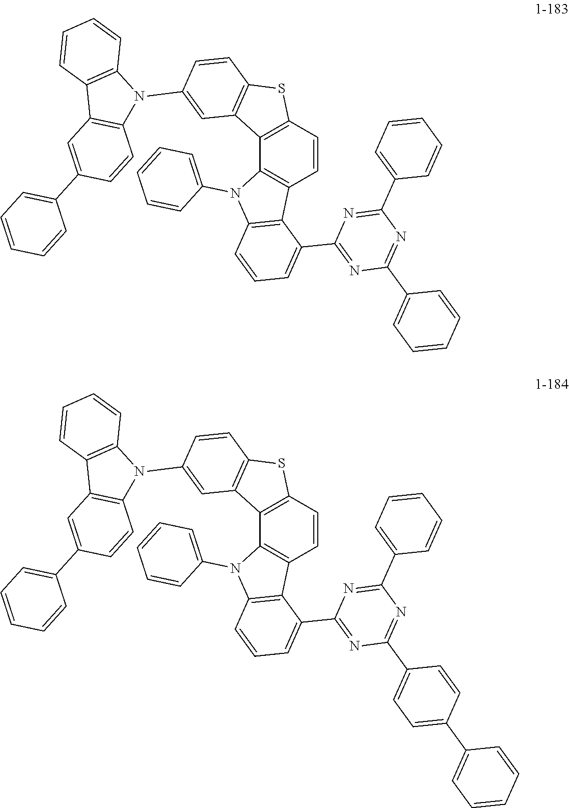 [SMILES: c1ccc(-c2ccc3c(c2)c2ccccc2n3-c2ccc3sc4ccc5c6c(-c7nc(-c8ccccc8)nc(-c8ccccc8)n7)cccc6n(-c6ccccc6)c5c4c3c2)cc1 ; c1ccc(-c2ccc(-c3nc(-c4ccccc4)nc(-c4cccc5c4c4ccc6sc7ccc(-n8c9ccccc9c9cc(-c%10ccccc%10)ccc98)cc7c6c4n5-c4ccccc4)n3)cc2)cc1]

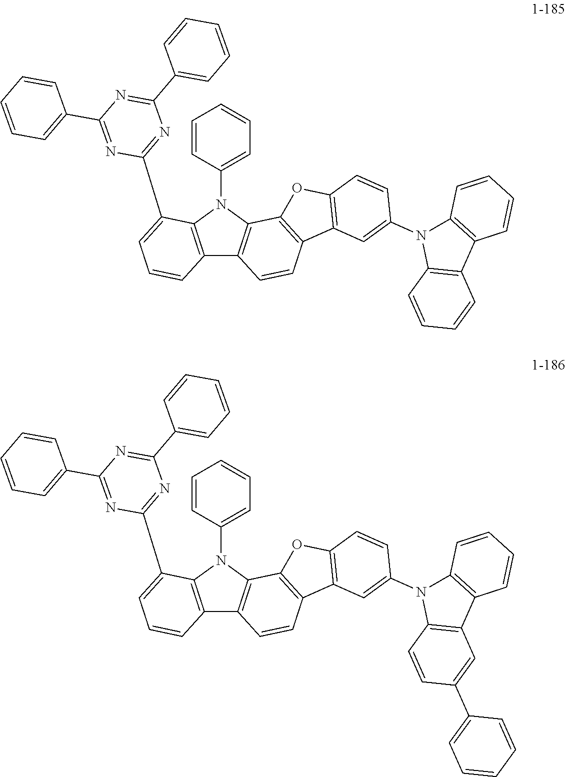 [SMILES: c1ccc(-c2nc(-c3ccccc3)nc(-c3cccc4c5ccc6c7cc(-n8c9ccccc9c9ccccc98)ccc7oc6c5n(-c5ccccc5)c34)n2)cc1 ; c1ccc(-c2ccc3c(c2)c2ccccc2n3-c2ccc3oc4c(ccc5c6cccc(-c7nc(-c8ccccc8)nc(-c8ccccc8)n7)c6n(-c6ccccc6)c54)c3c2)cc1]

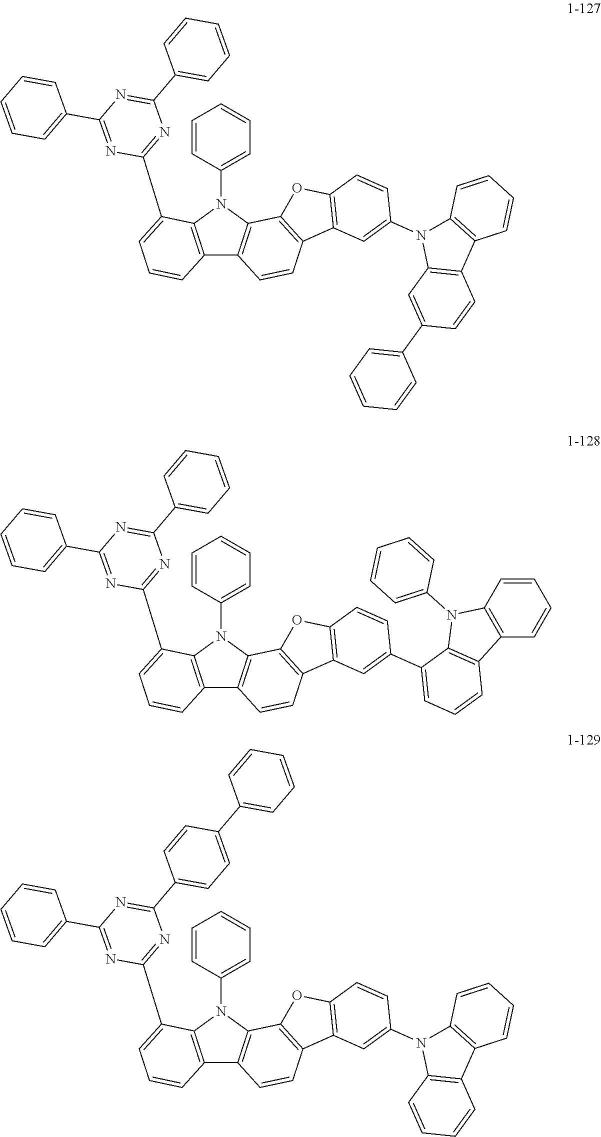

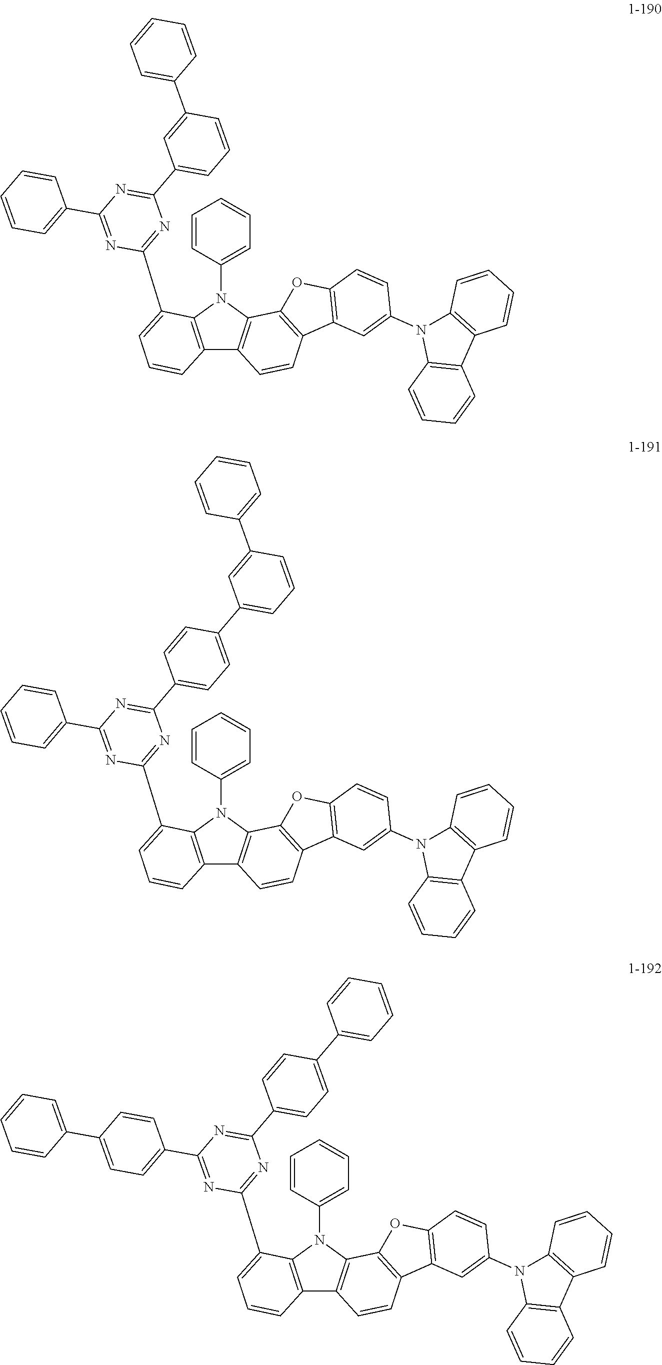 [SMILES: c1ccc(-c2cccc(-c3nc(-c4ccccc4)nc(-c4cccc5c6ccc7c8cc(-n9c%10ccccc%10c%10ccccc%109)ccc8oc7c6n(-c6ccccc6)c45)n3)c2)cc1 ; c1ccc(-c2cccc(-c3ccc(-c4nc(-c5ccccc5)nc(-c5cccc6c7ccc8c9cc(-n%10c%11ccccc%11c%11ccccc%11%10)ccc9oc8c7n(-c7ccccc7)c56)n4)cc3)c2)cc1 ; c1ccc(-c2ccc(-c3nc(-c4ccc(-c5ccccc5)cc4)nc(-c4cccc5c6ccc7c8cc(-n9c%10ccccc%10c%10ccccc%109)ccc8oc7c6n(-c6ccccc6)c45)n3)cc2)cc1]

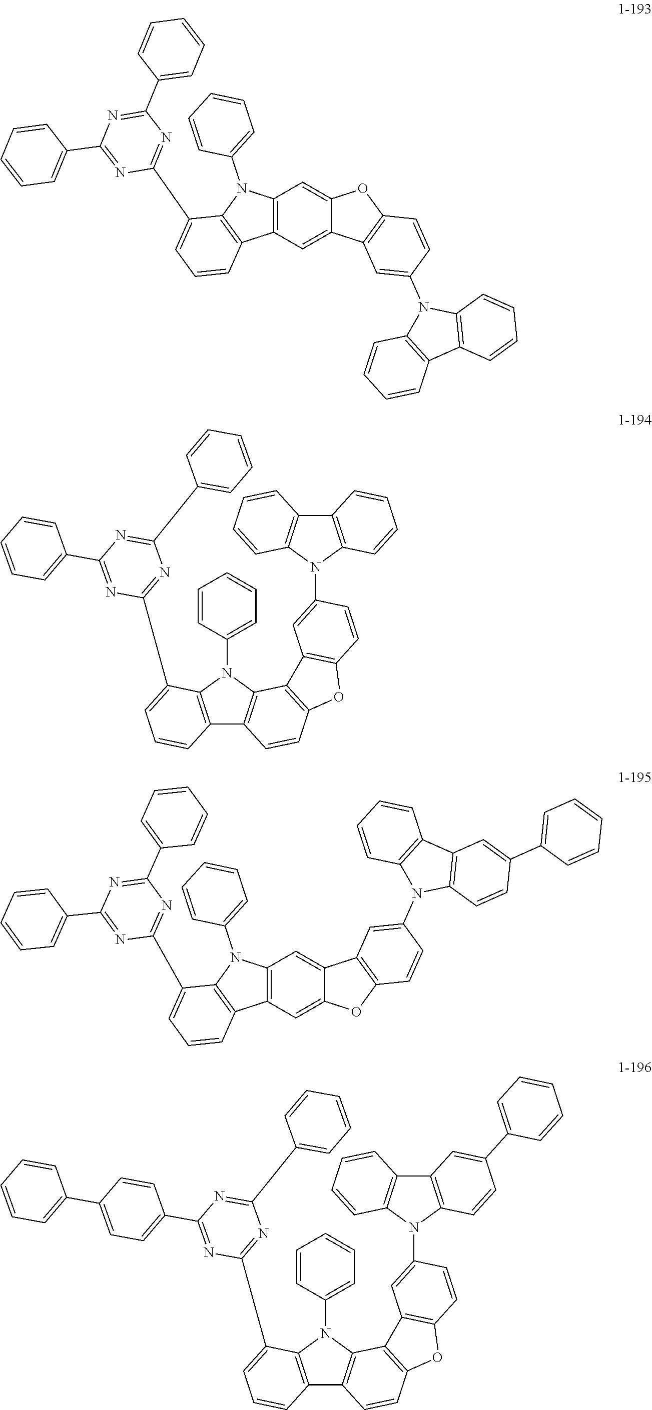

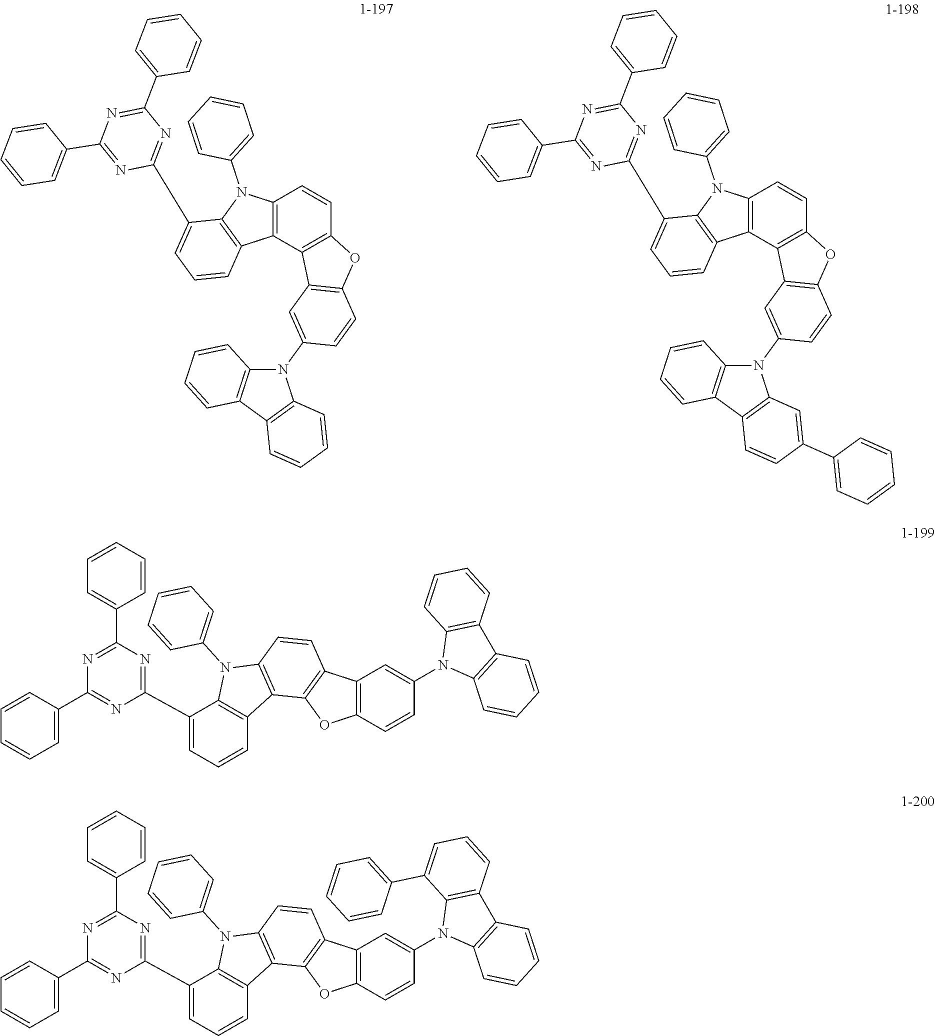 [SMILES: c1ccc(-c2nc(-c3ccccc3)nc(-c3cccc4c5c6c(ccc5n(-c5ccccc5)c34)oc3ccc(-n4c5ccccc5c5ccccc54)cc36)n2)cc1 ; c1ccc(-c2ccc3c4ccccc4n(-c4ccc5oc6ccc7c(c8cccc(-c9nc(-c%10ccccc%10)nc(-c%10ccccc%10)n9)c8n7-c7ccccc7)c6c5c4)c3c2)cc1 ; c1ccc(-c2nc(-c3ccccc3)nc(-c3cccc4c5c6oc7ccc(-n8c9ccccc9c9ccccc98)cc7c6ccc5n(-c5ccccc5)c34)n2)cc1 ; c1ccc(-c2nc(-c3ccccc3)nc(-c3cccc4c5c6oc7ccc(-n8c9ccccc9c9cccc(-c%10ccccc%10)c98)cc7c6ccc5n(-c5ccccc5)c34)n2)cc1]

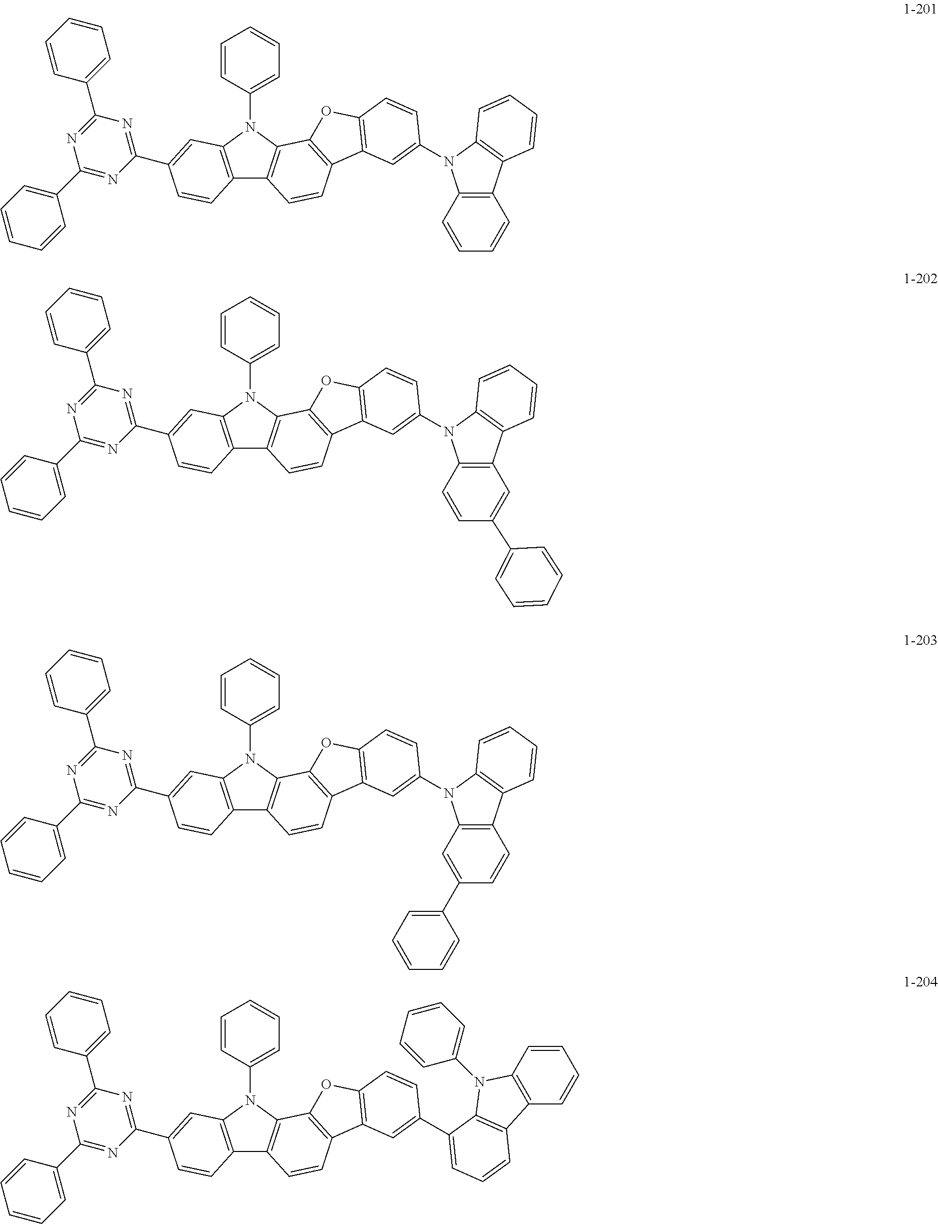

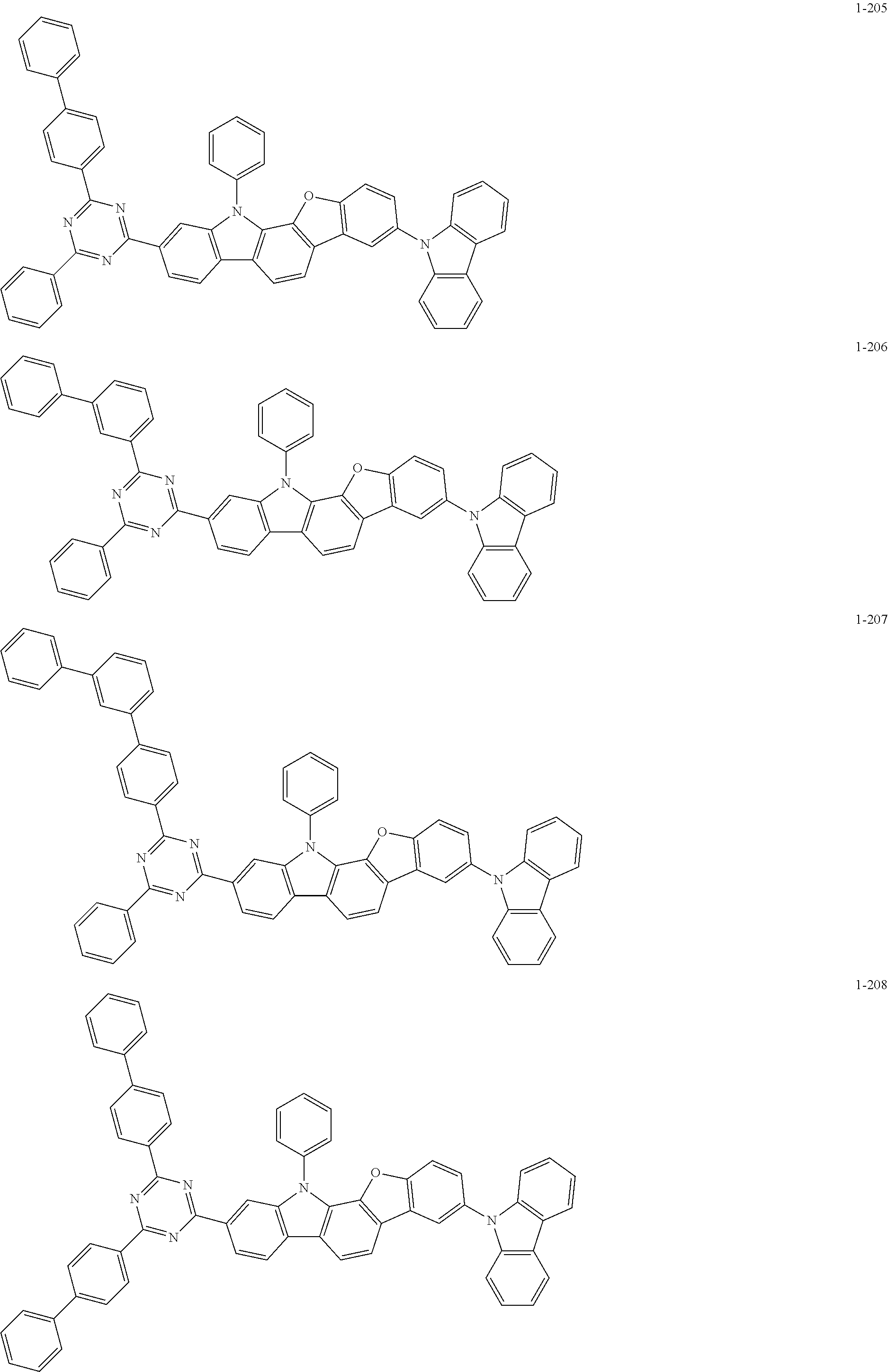

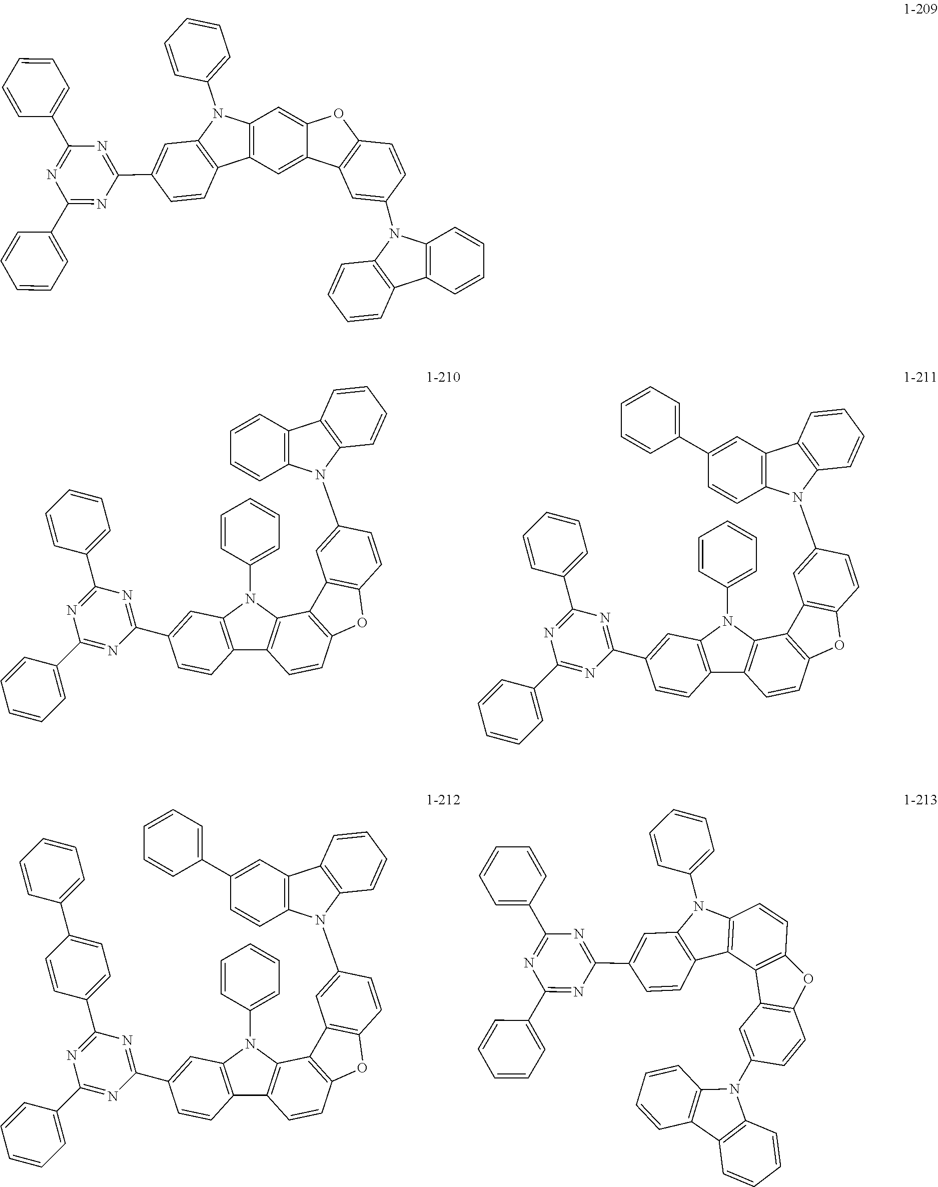

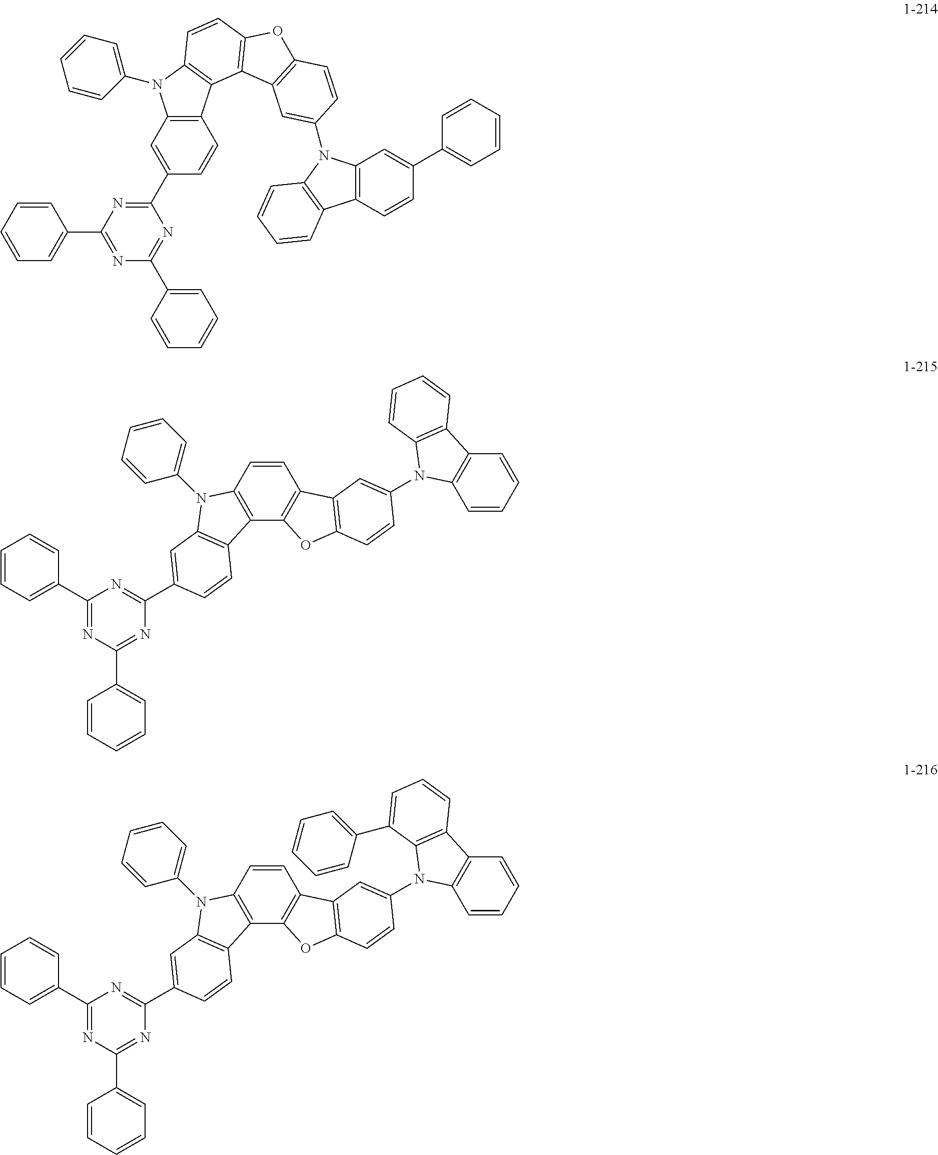 [SMILES: c1ccc(-c2ccc3c4ccccc4n(-c4ccc5oc6ccc7c(c8ccc(-c9nc(-c%10ccccc%10)nc(-c%10ccccc%10)n9)cc8n7-c7ccccc7)c6c5c4)c3c2)cc1 ; c1ccc(-c2nc(-c3ccccc3)nc(-c3ccc4c5c6oc7ccc(-n8c9ccccc9c9ccccc98)cc7c6ccc5n(-c5ccccc5)c4c3)n2)cc1 ; c1ccc(-c2nc(-c3ccccc3)nc(-c3ccc4c5c6oc7ccc(-n8c9ccccc9c9cccc(-c%10ccccc%10)c98)cc7c6ccc5n(-c5ccccc5)c4c3)n2)cc1]

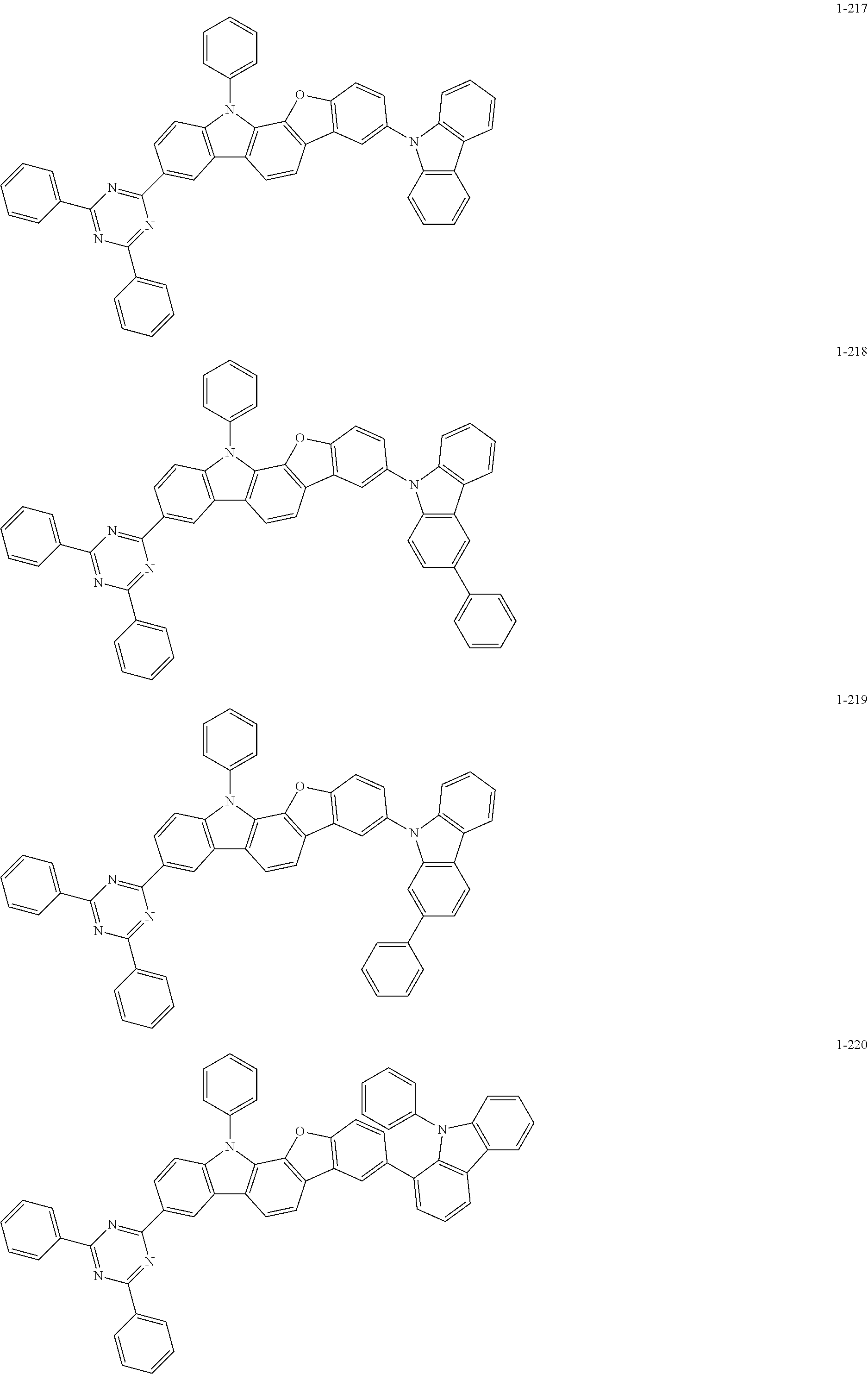 [SMILES: c1ccc(-c2nc(-c3ccccc3)nc(-c3ccc4c(c3)c3ccc5c6cc(-n7c8ccccc8c8ccccc87)ccc6oc5c3n4-c3ccccc3)n2)cc1 ; c1ccc(-c2ccc3c(c2)c2ccccc2n3-c2ccc3oc4c(ccc5c6cc(-c7nc(-c8ccccc8)nc(-c8ccccc8)n7)ccc6n(-c6ccccc6)c54)c3c2)cc1 ; c1ccc(-c2ccc3c4ccccc4n(-c4ccc5oc6c(ccc7c8cc(-c9nc(-c%10ccccc%10)nc(-c%10ccccc%10)n9)ccc8n(-c8ccccc8)c76)c5c4)c3c2)cc1 ; c1ccc(-c2nc(-c3ccccc3)nc(-c3ccc4c(c3)c3ccc5c6cc(-c7cccc8c9ccccc9n(-c9ccccc9)c78)ccc6oc5c3n4-c3ccccc3)n2)cc1]

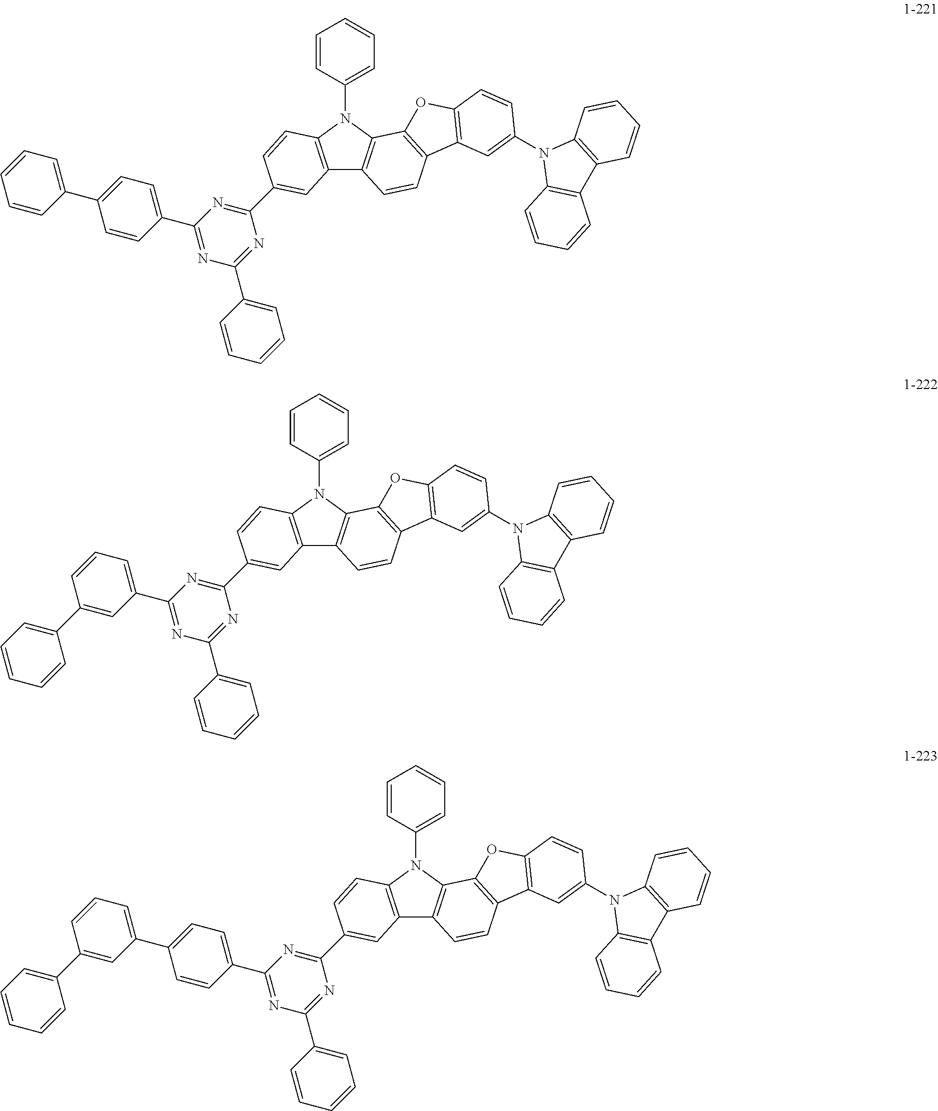 [SMILES: c1ccc(-c2ccc(-c3nc(-c4ccccc4)nc(-c4ccc5c(c4)c4ccc6c7cc(-n8c9ccccc9c9ccccc98)ccc7oc6c4n5-c4ccccc4)n3)cc2)cc1 ; c1ccc(-c2cccc(-c3nc(-c4ccccc4)nc(-c4ccc5c(c4)c4ccc6c7cc(-n8c9ccccc9c9ccccc98)ccc7oc6c4n5-c4ccccc4)n3)c2)cc1 ; c1ccc(-c2cccc(-c3ccc(-c4nc(-c5ccccc5)nc(-c5ccc6c(c5)c5ccc7c8cc(-n9c%10ccccc%10c%10ccccc%109)ccc8oc7c5n6-c5ccccc5)n4)cc3)c2)cc1]

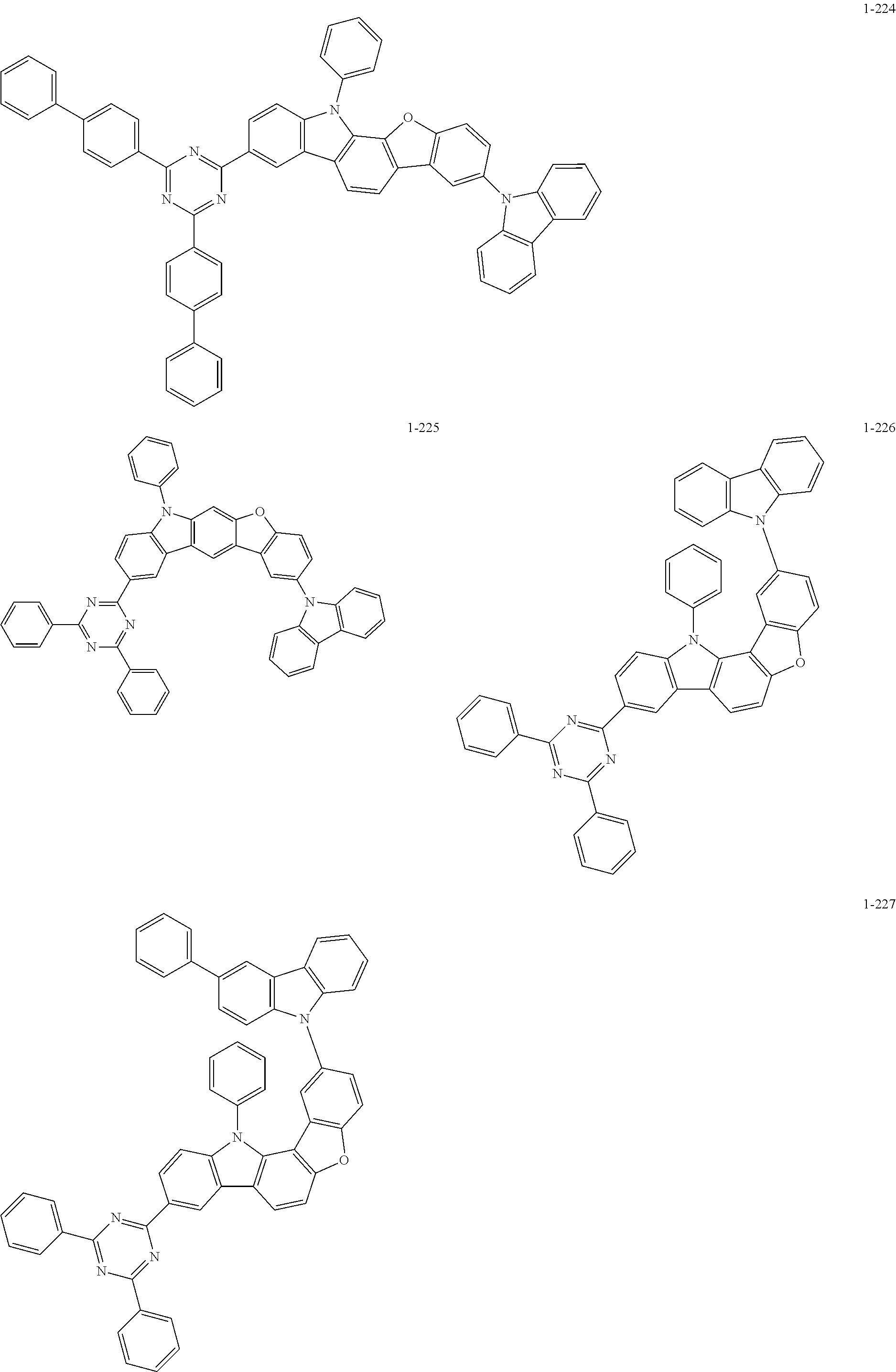

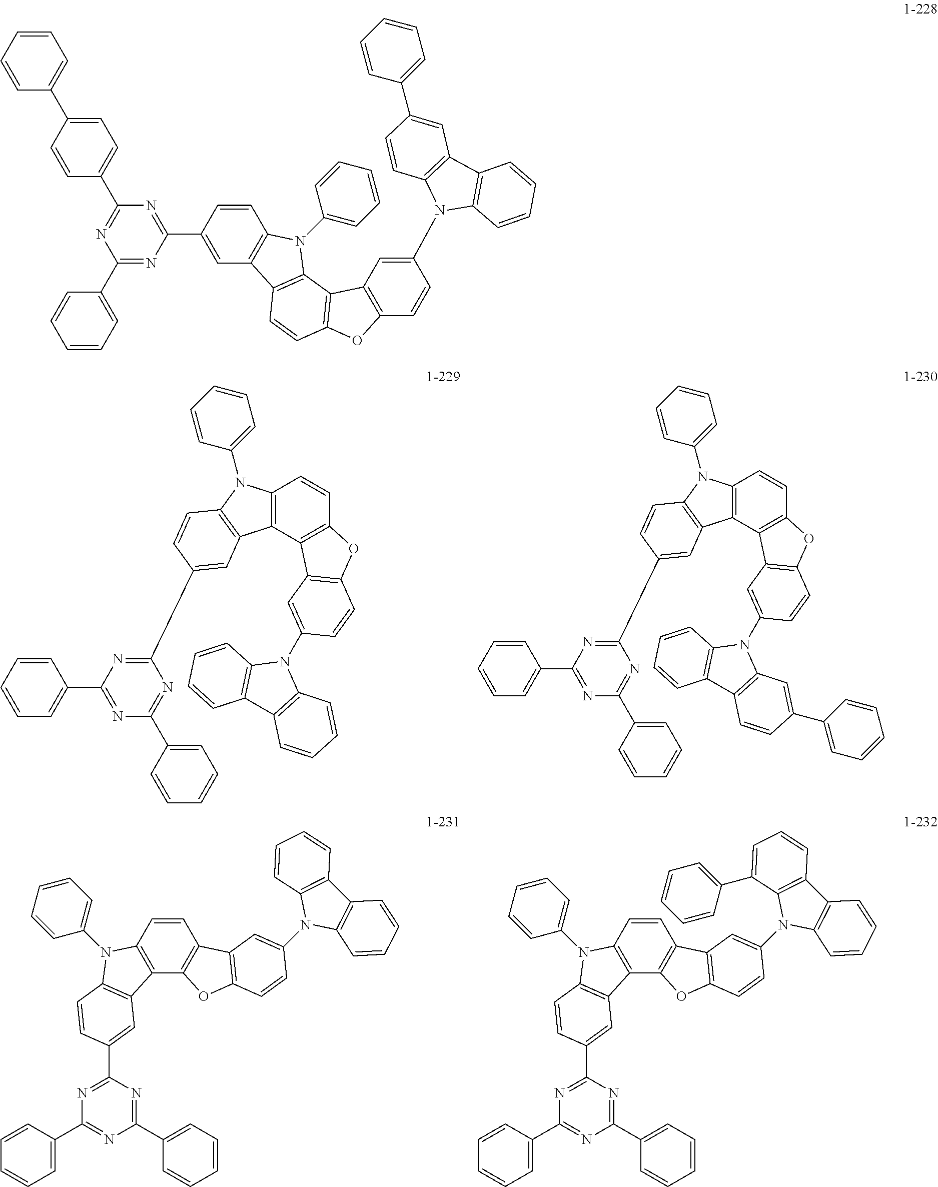

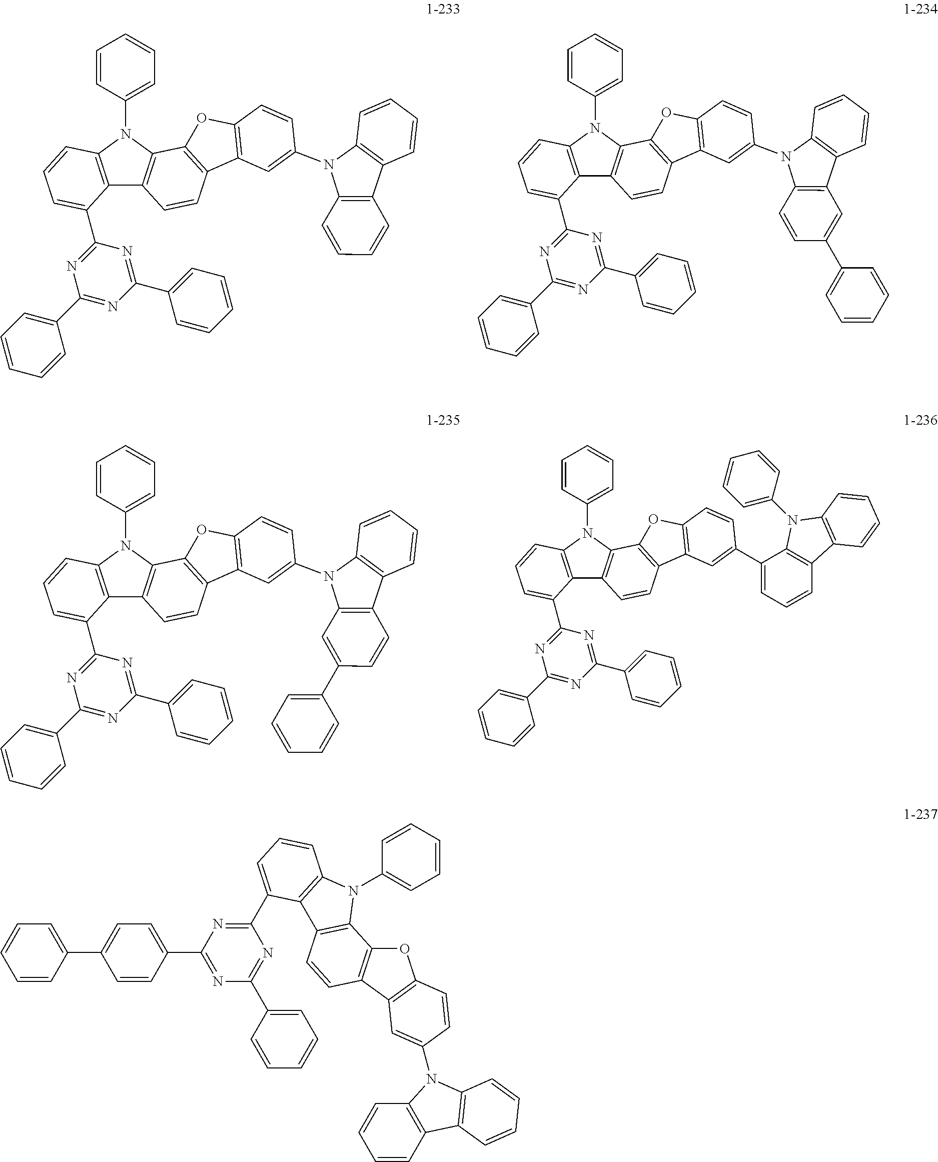

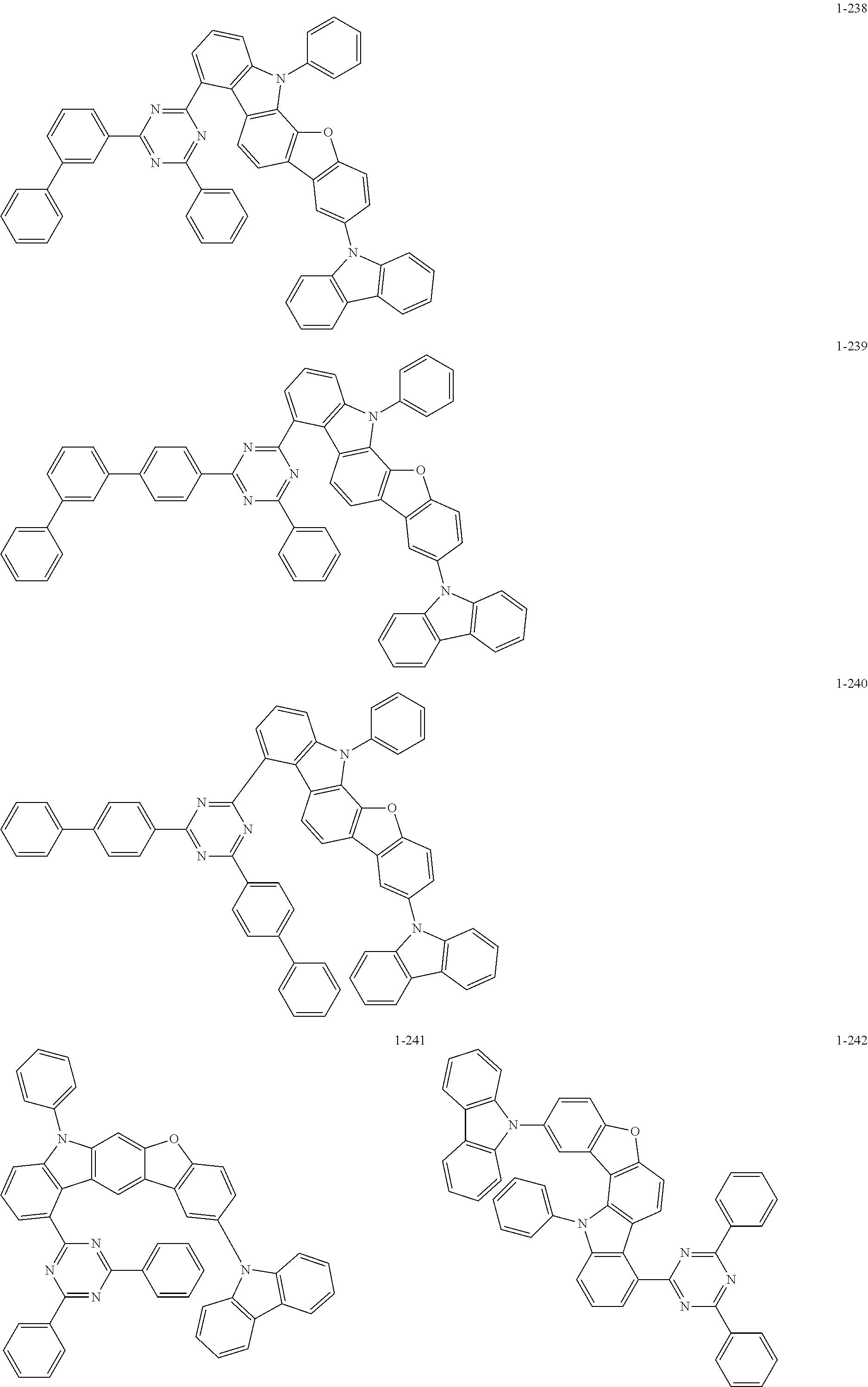

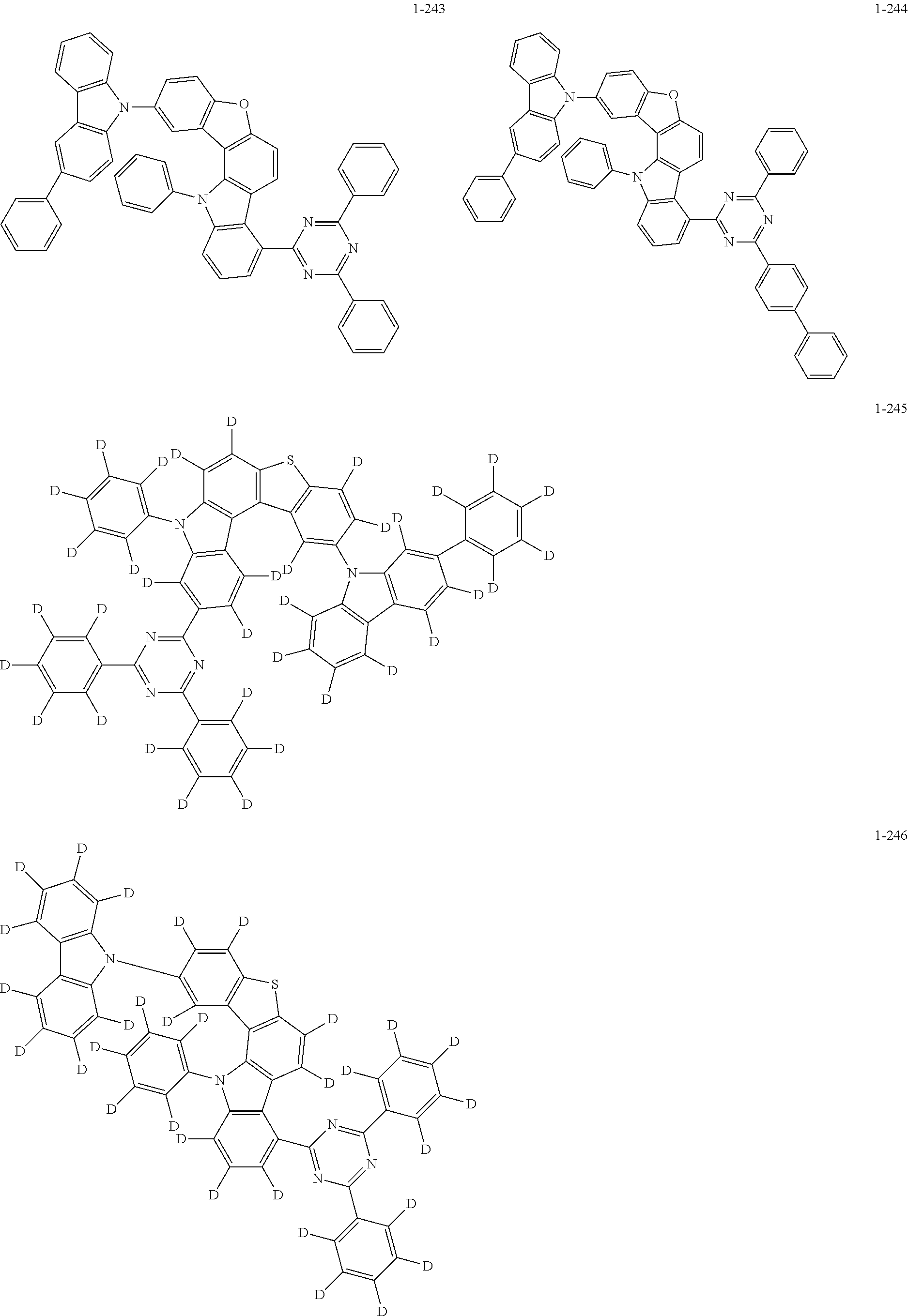

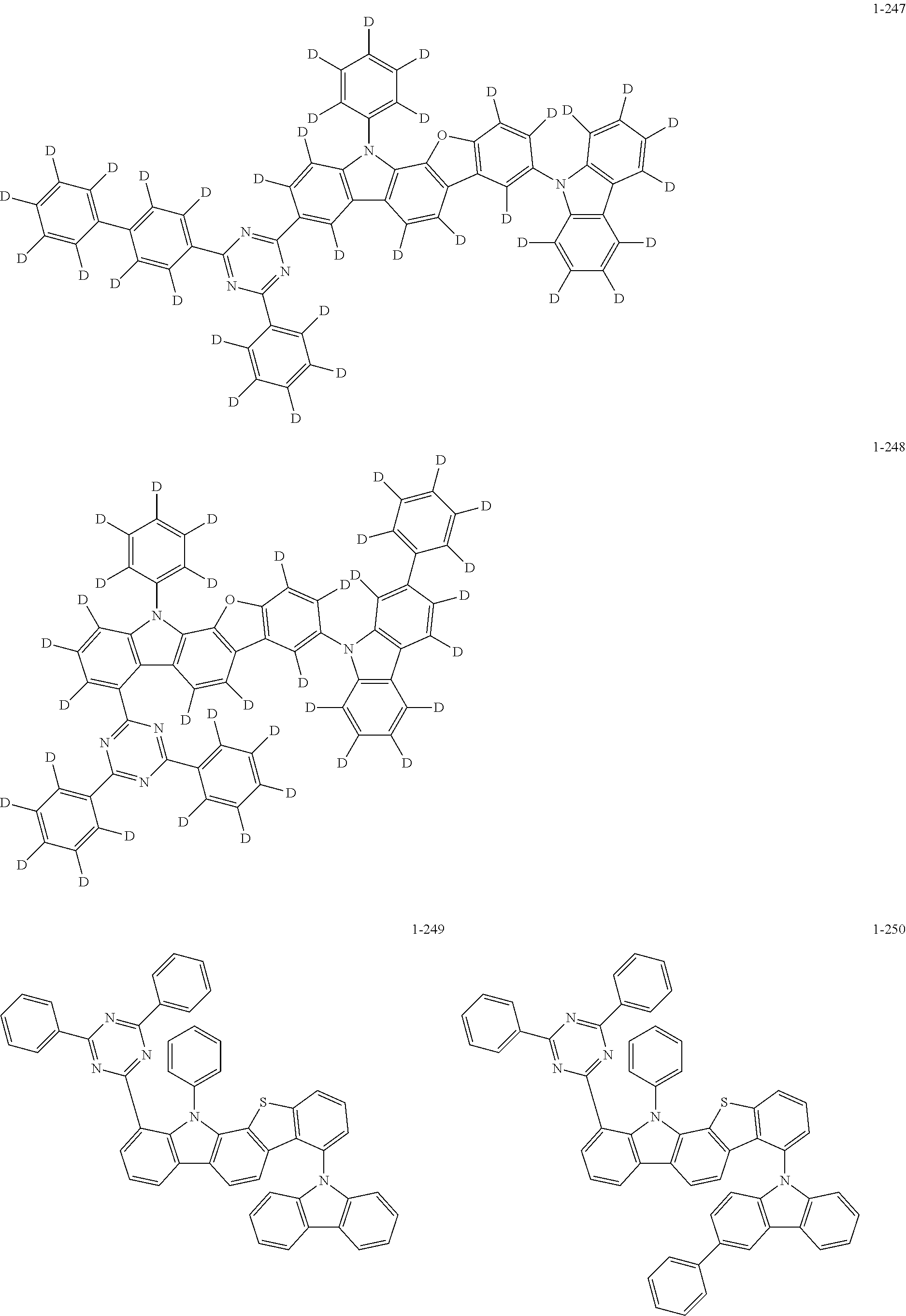

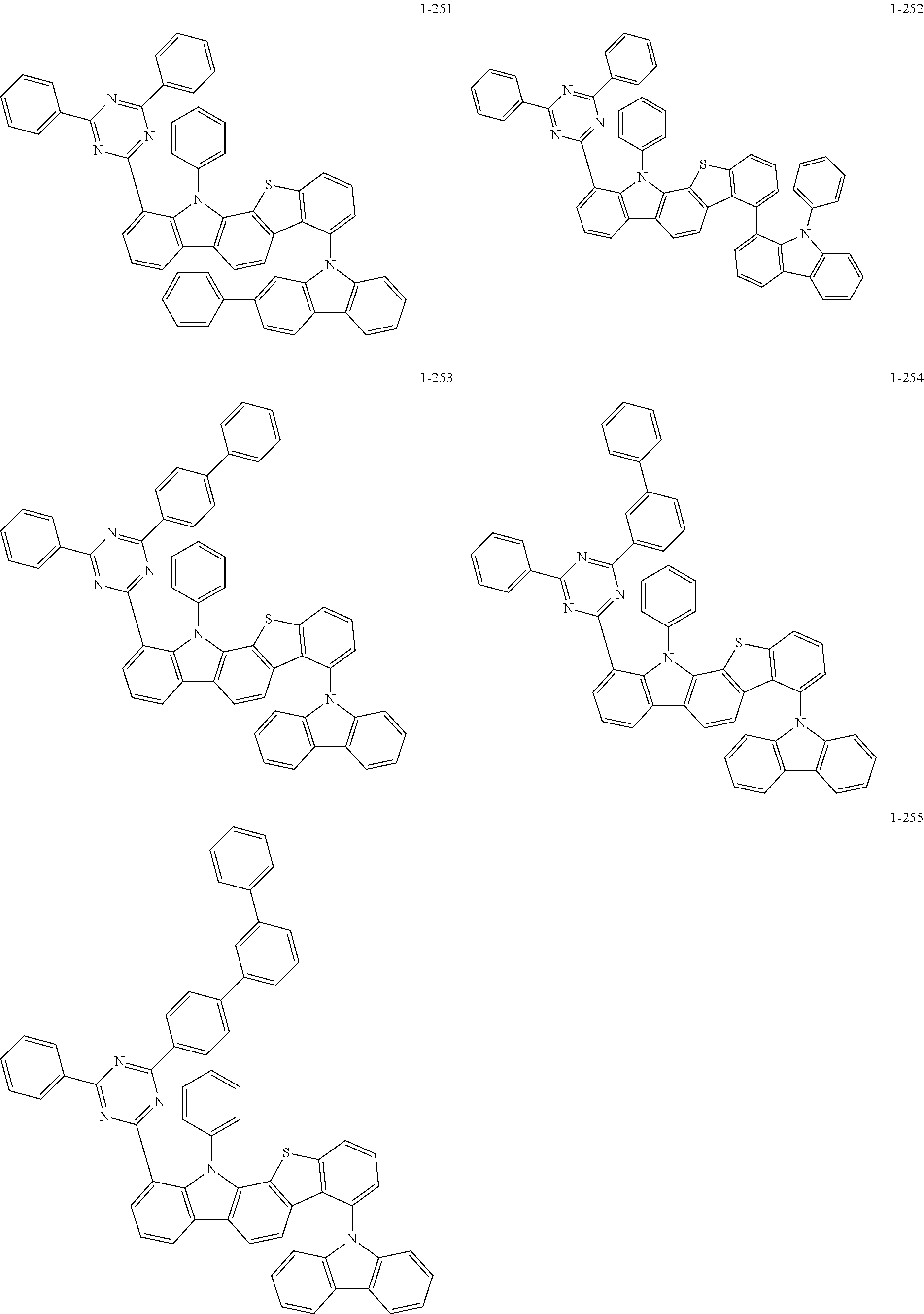

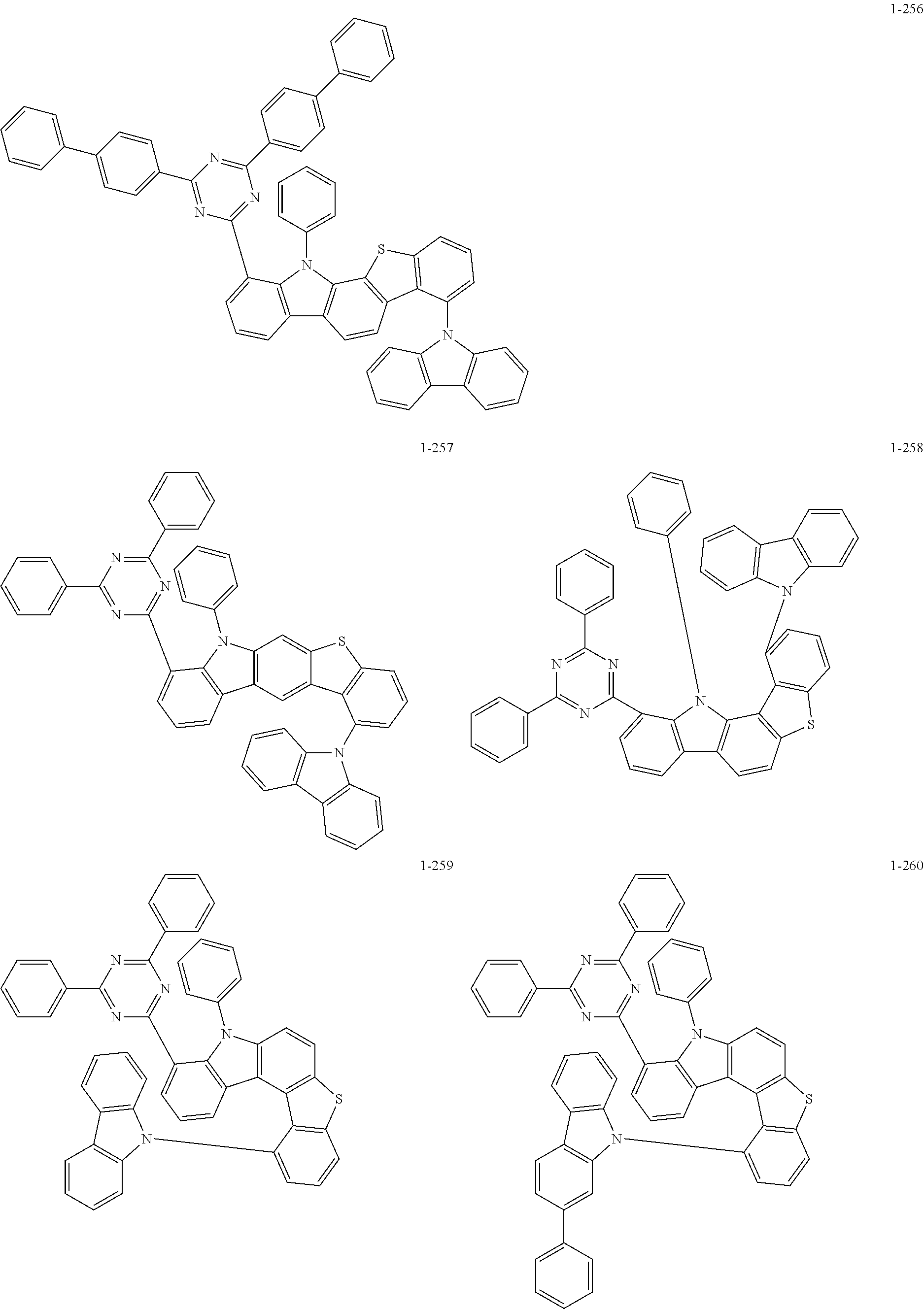

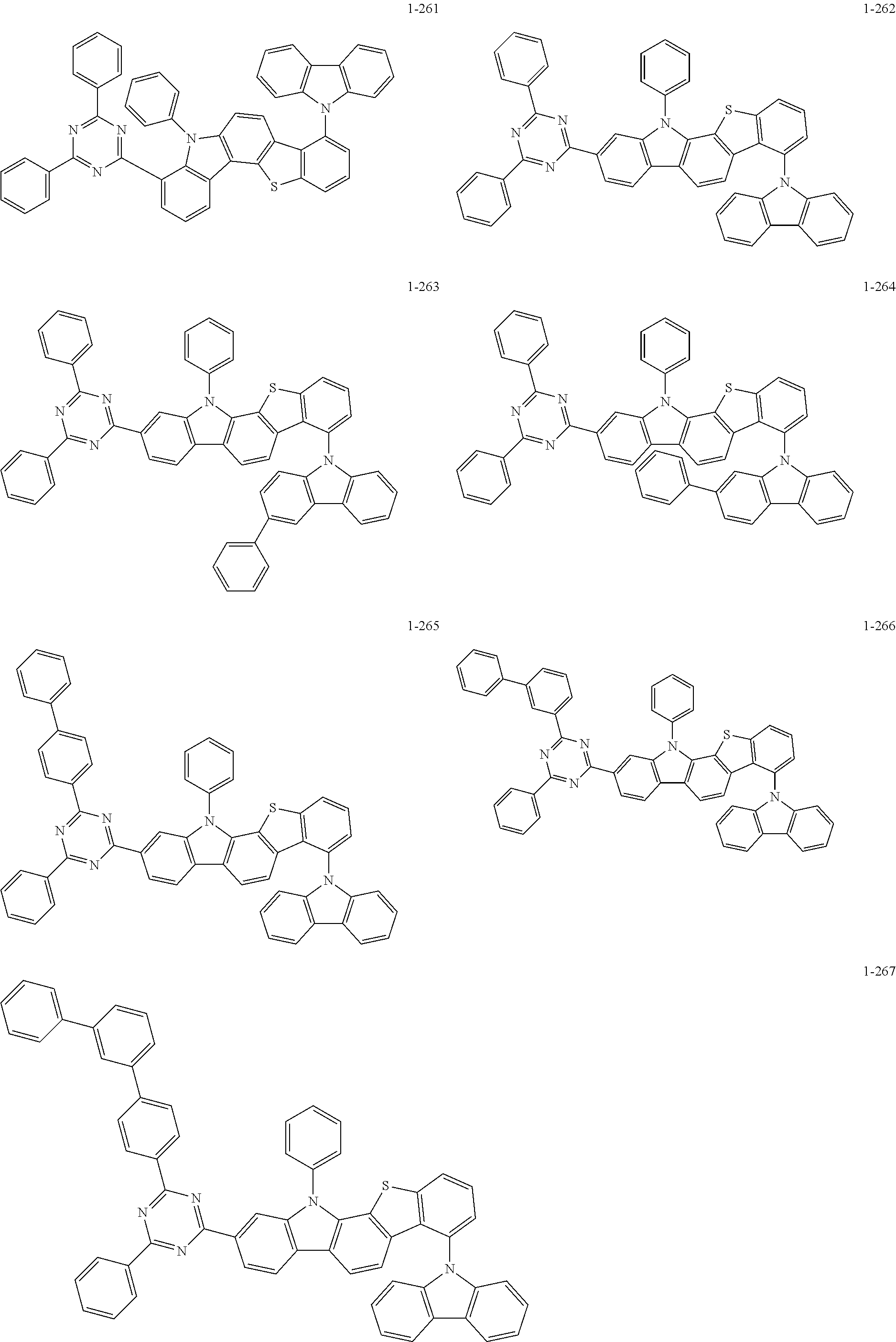

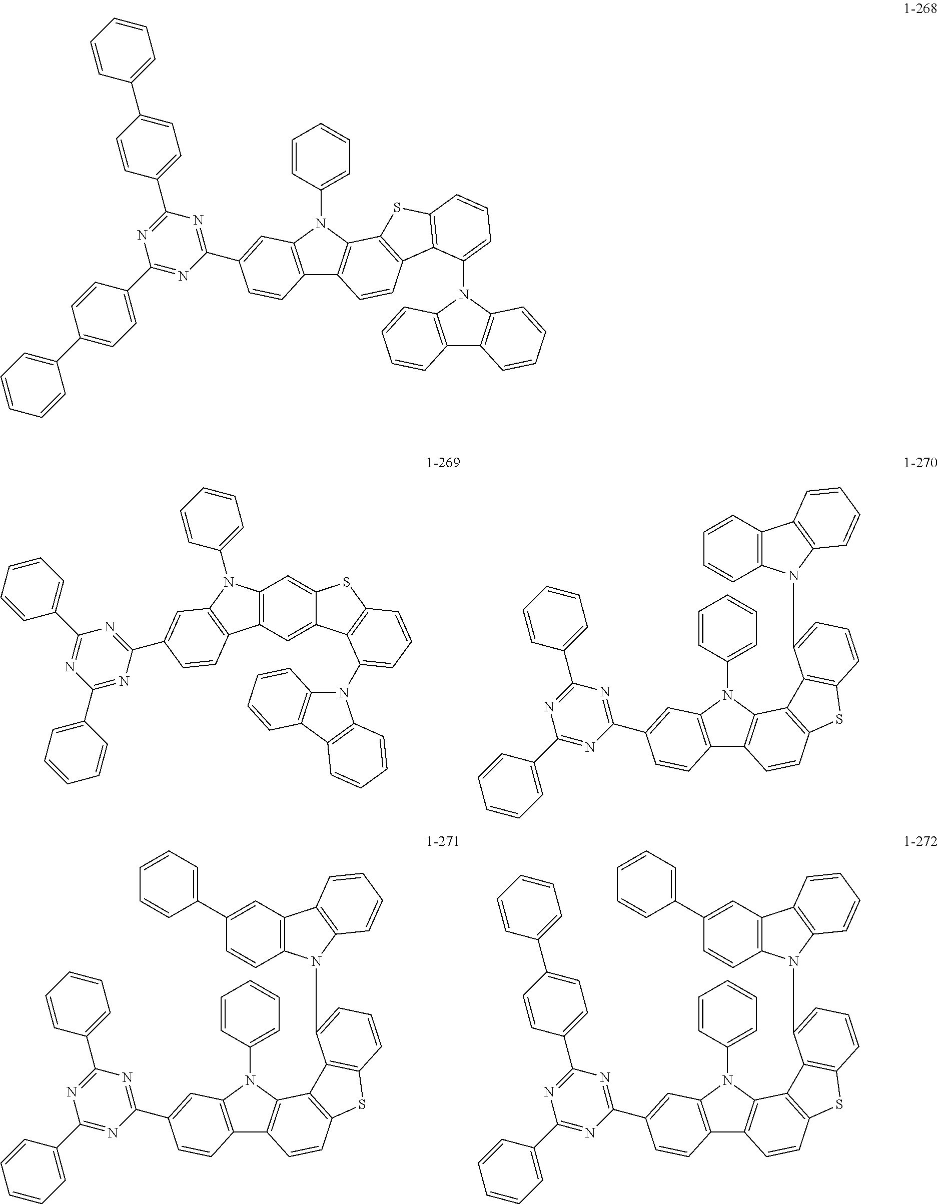

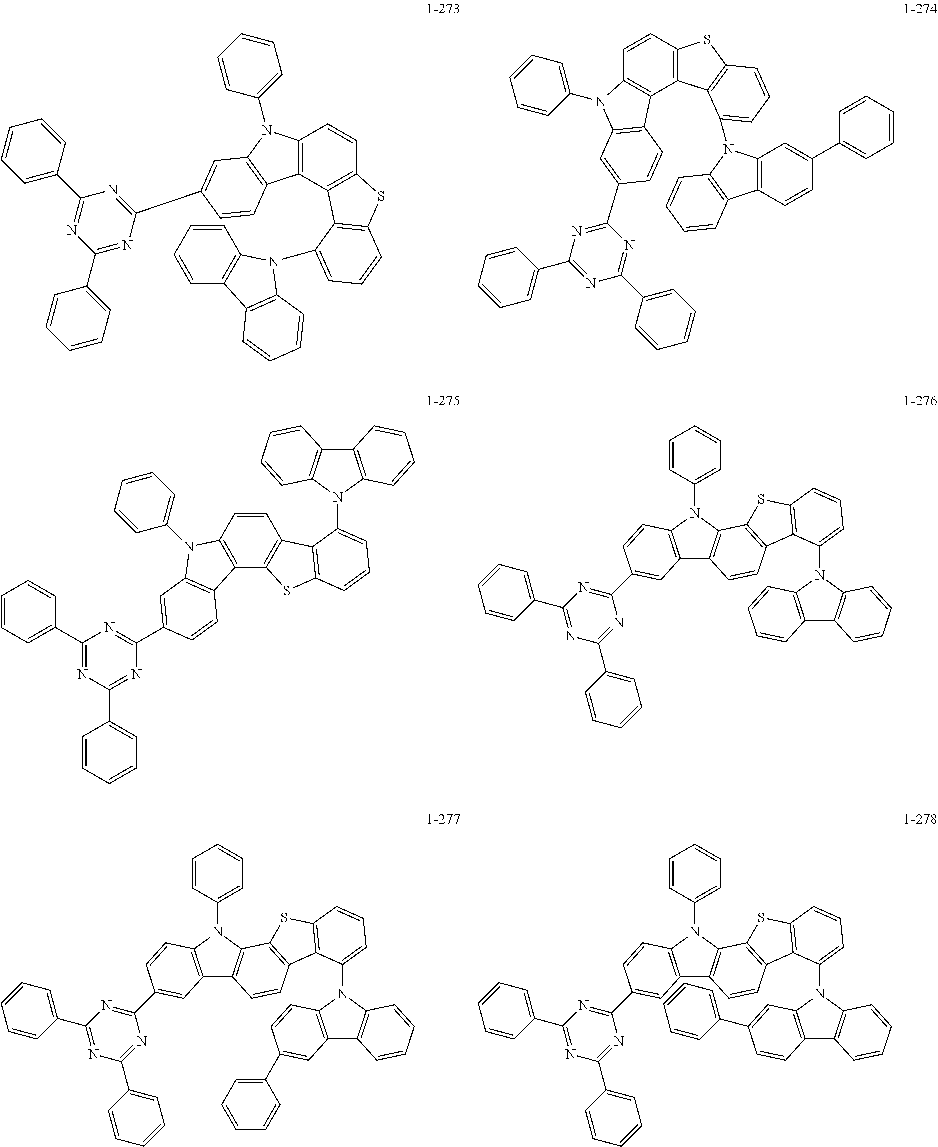

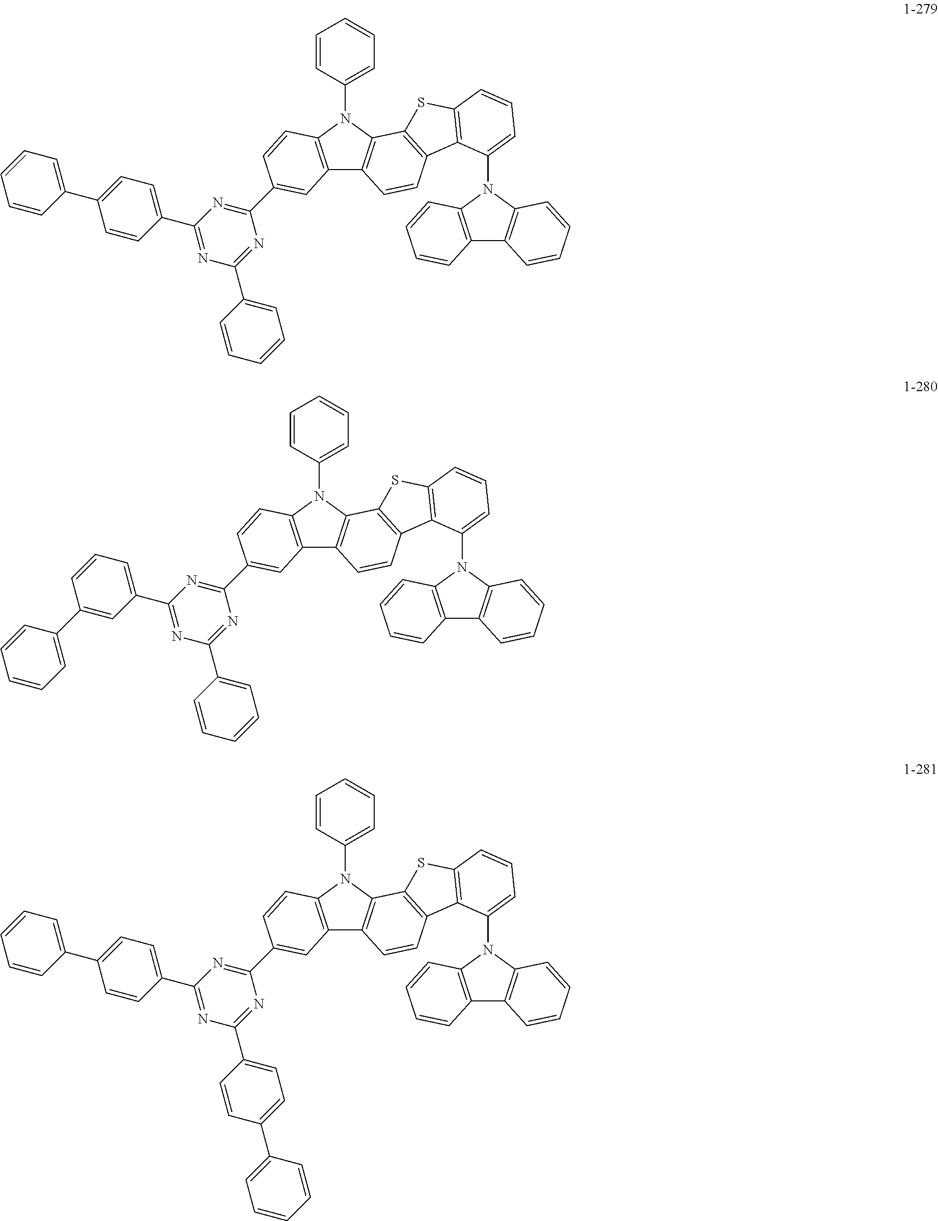 [SMILES: c1ccc(-c2ccc(-c3nc(-c4ccccc4)nc(-c4ccc5c(c4)c4ccc6c(sc7cccc(-n8c9ccccc9c9ccccc98)c76)c4n5-c4ccccc4)n3)cc2)cc1 ; c1ccc(-c2cccc(-c3nc(-c4ccccc4)nc(-c4ccc5c(c4)c4ccc6c(sc7cccc(-n8c9ccccc9c9ccccc98)c76)c4n5-c4ccccc4)n3)c2)cc1 ; c1ccc(-c2ccc(-c3nc(-c4ccc(-c5ccccc5)cc4)nc(-c4ccc5c(c4)c4ccc6c(sc7cccc(-n8c9ccccc9c9ccccc98)c76)c4n5-c4ccccc4)n3)cc2)cc1]

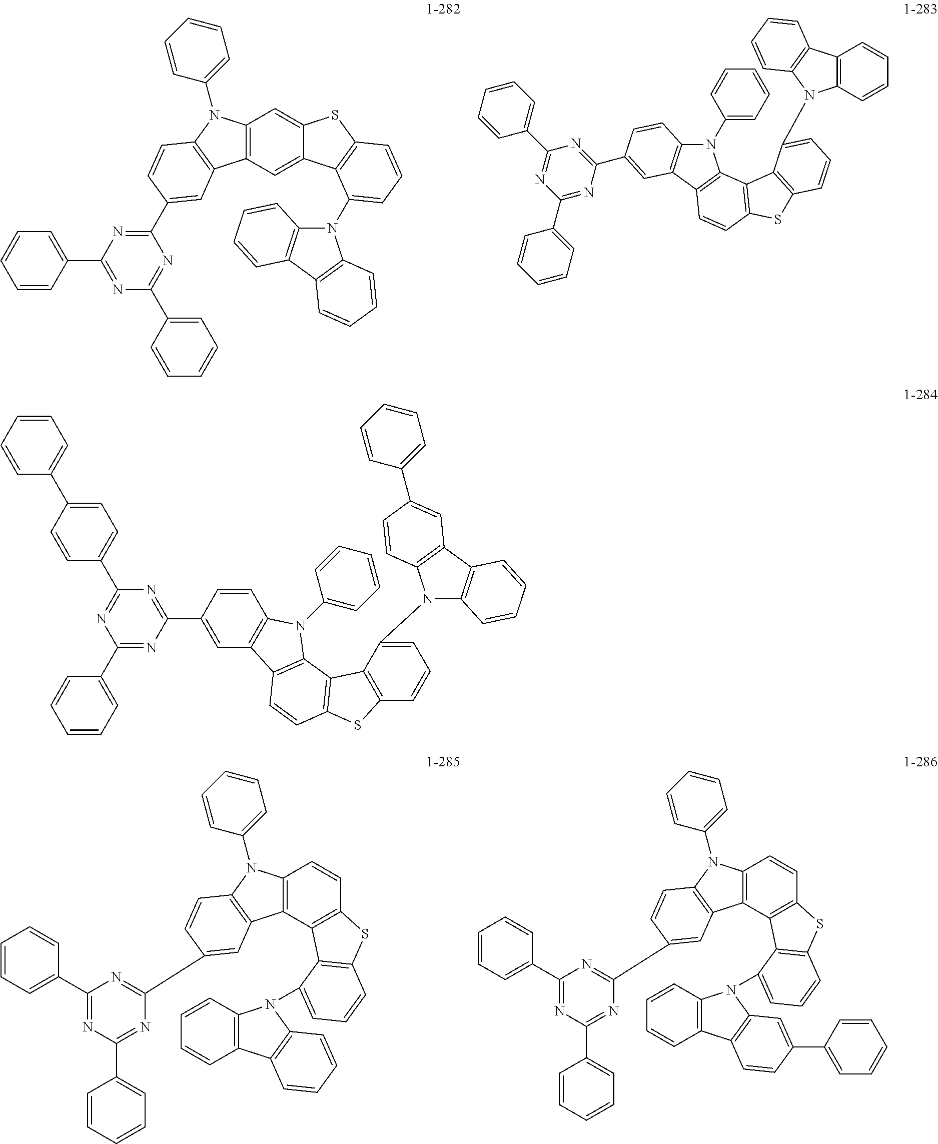

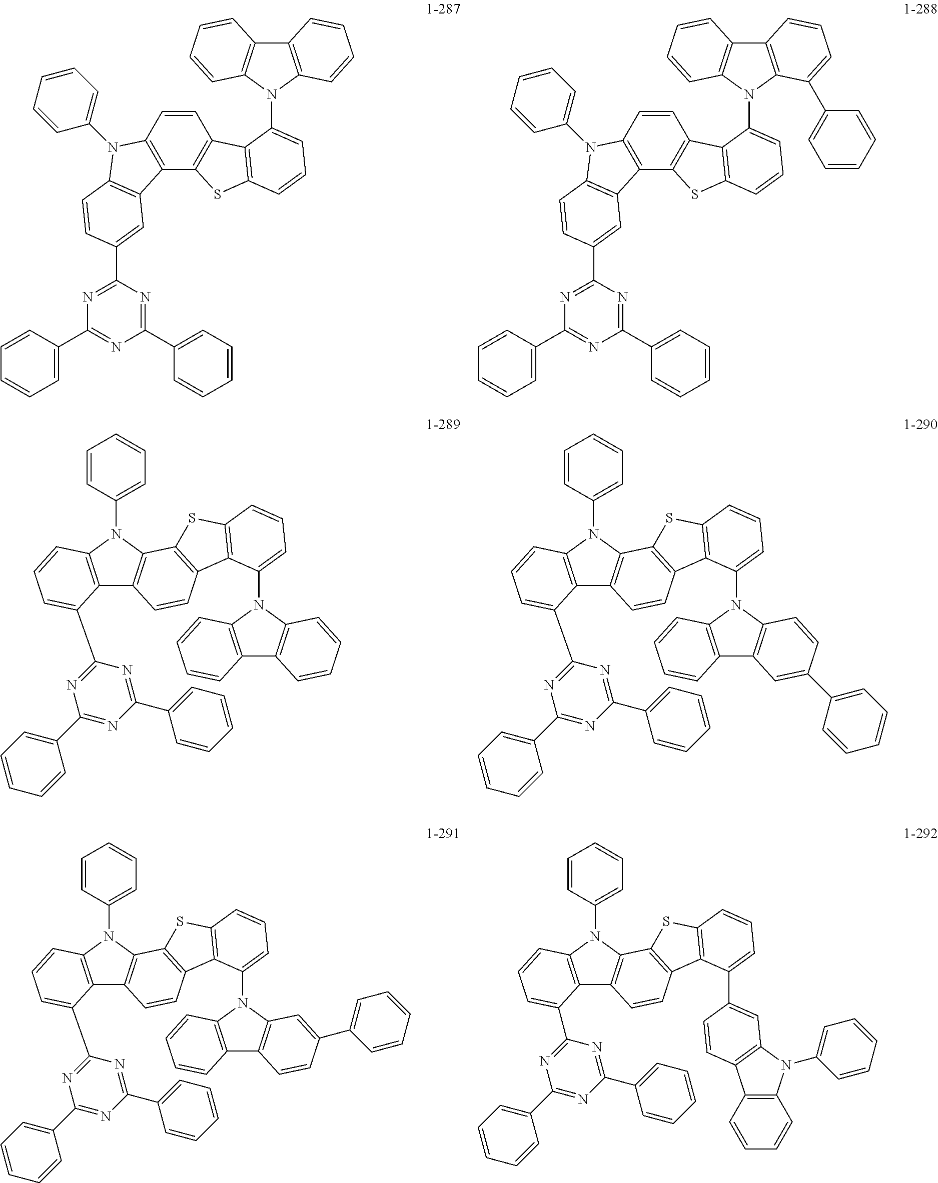

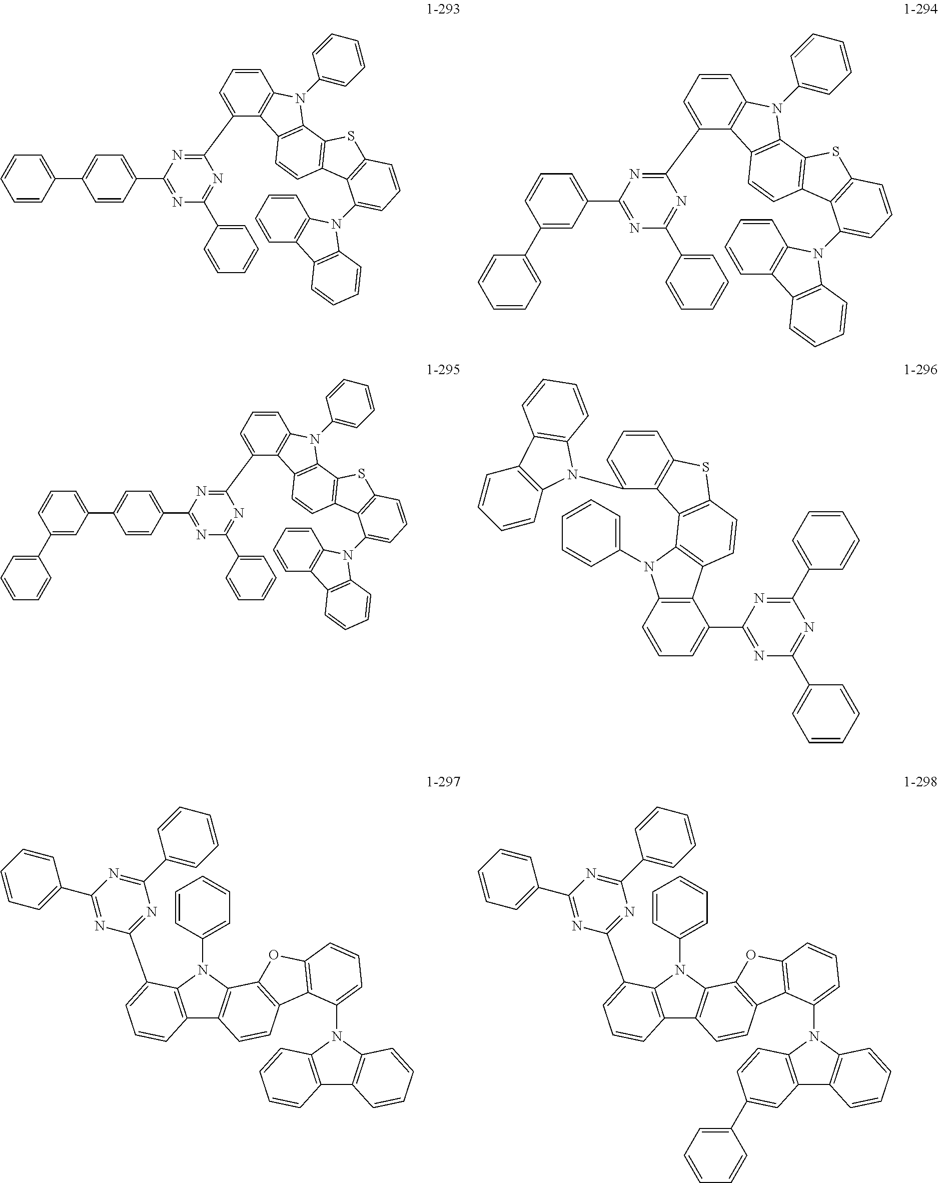

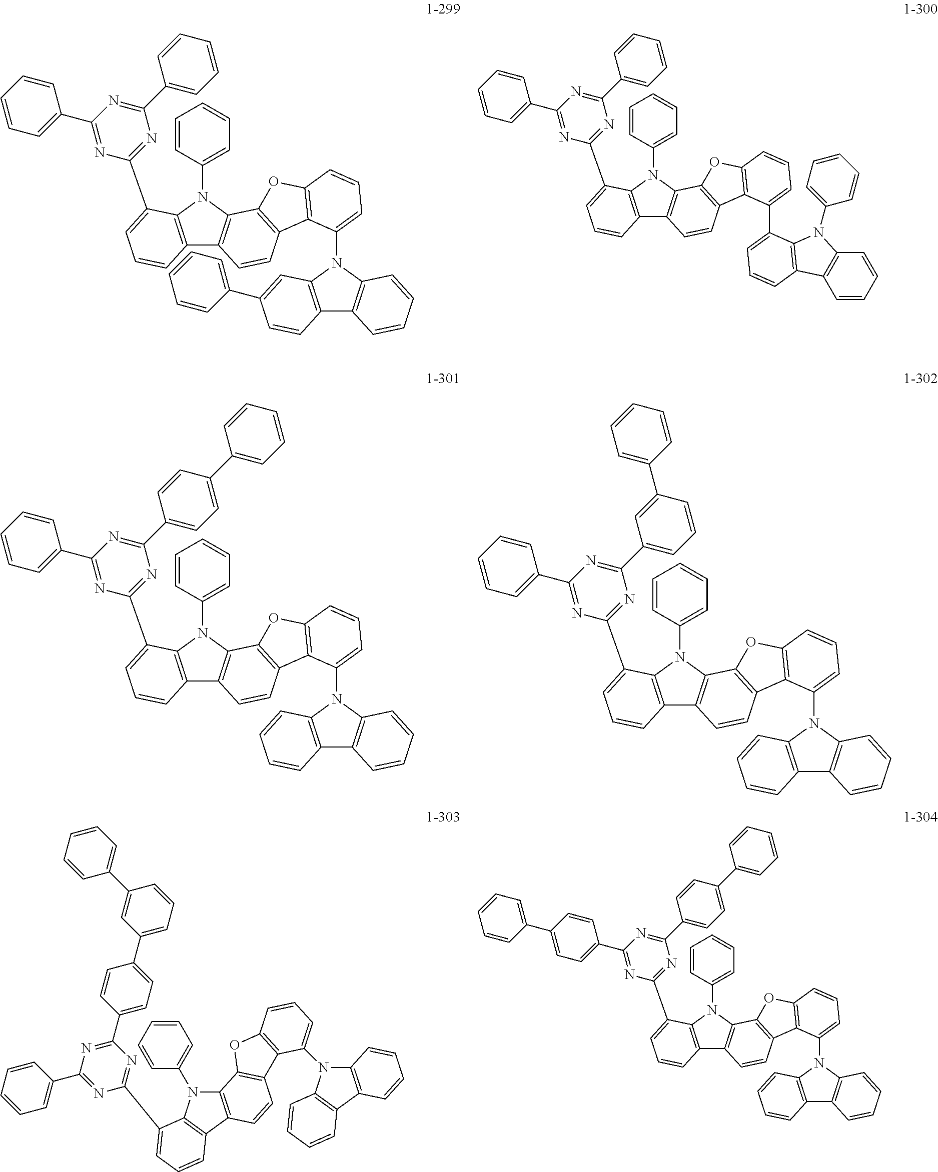

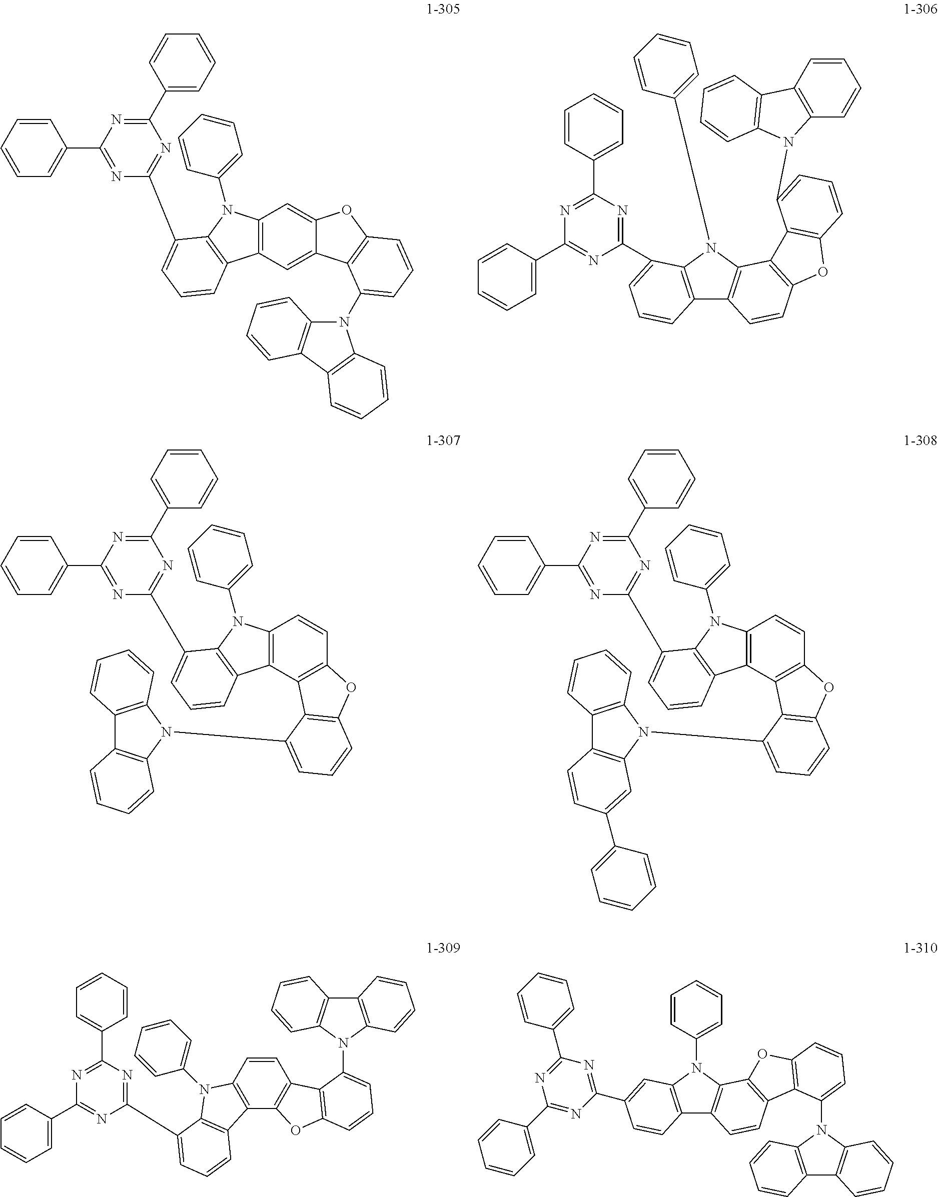

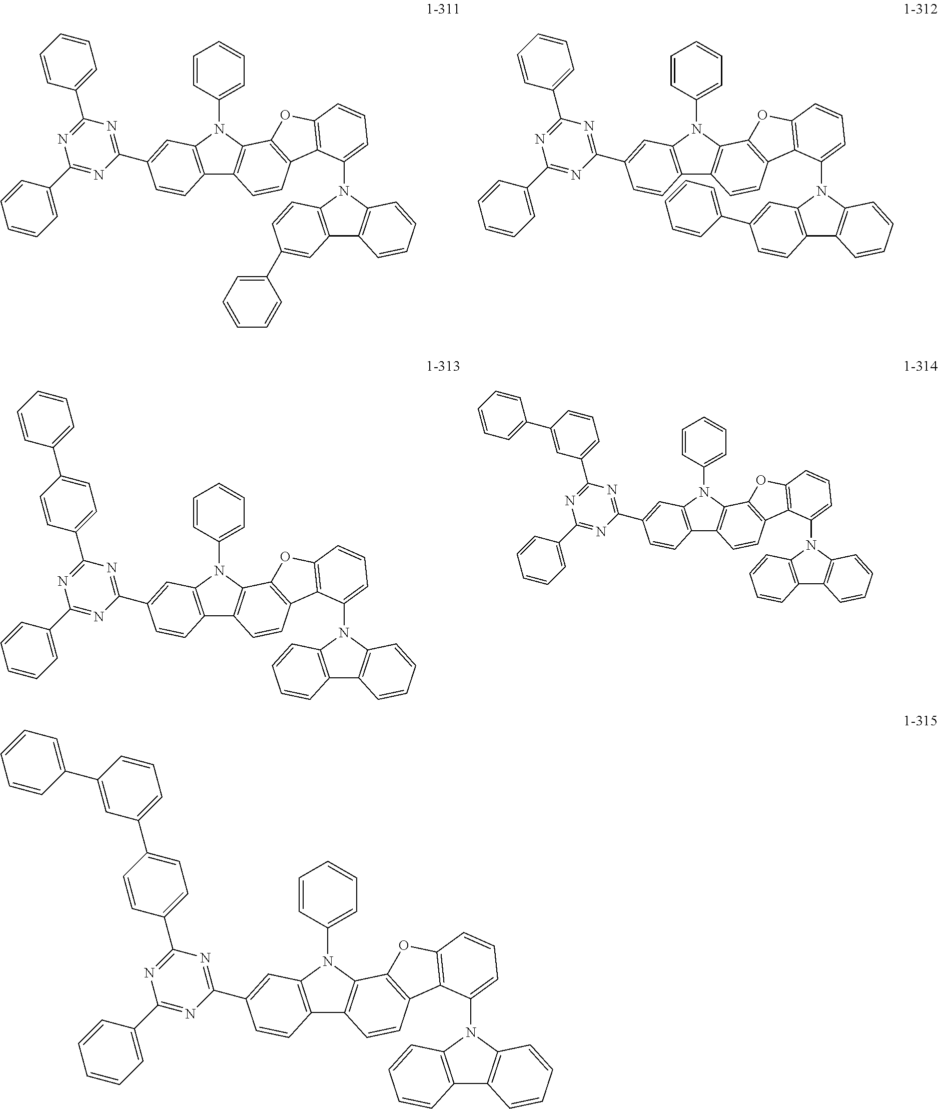

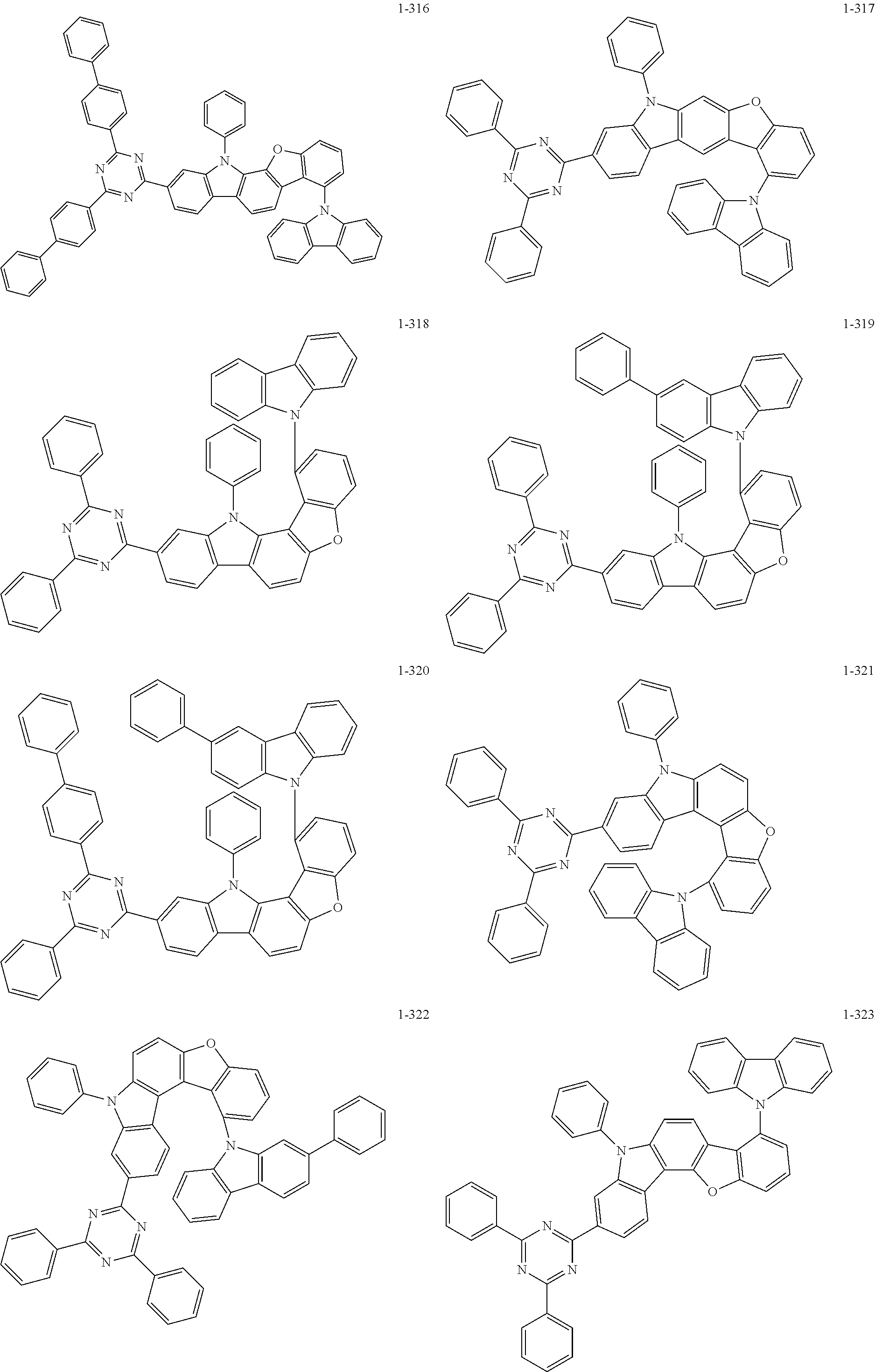

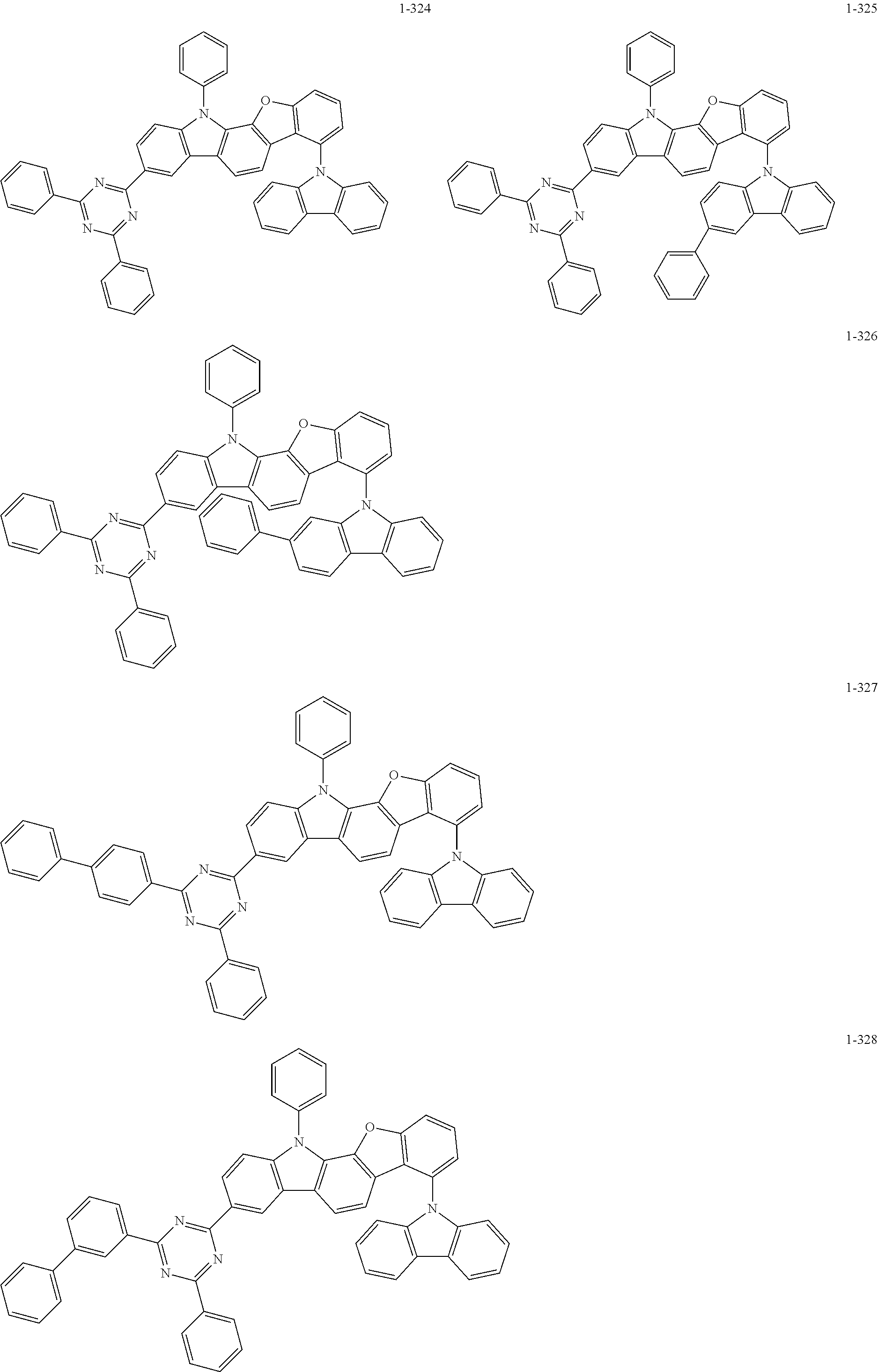

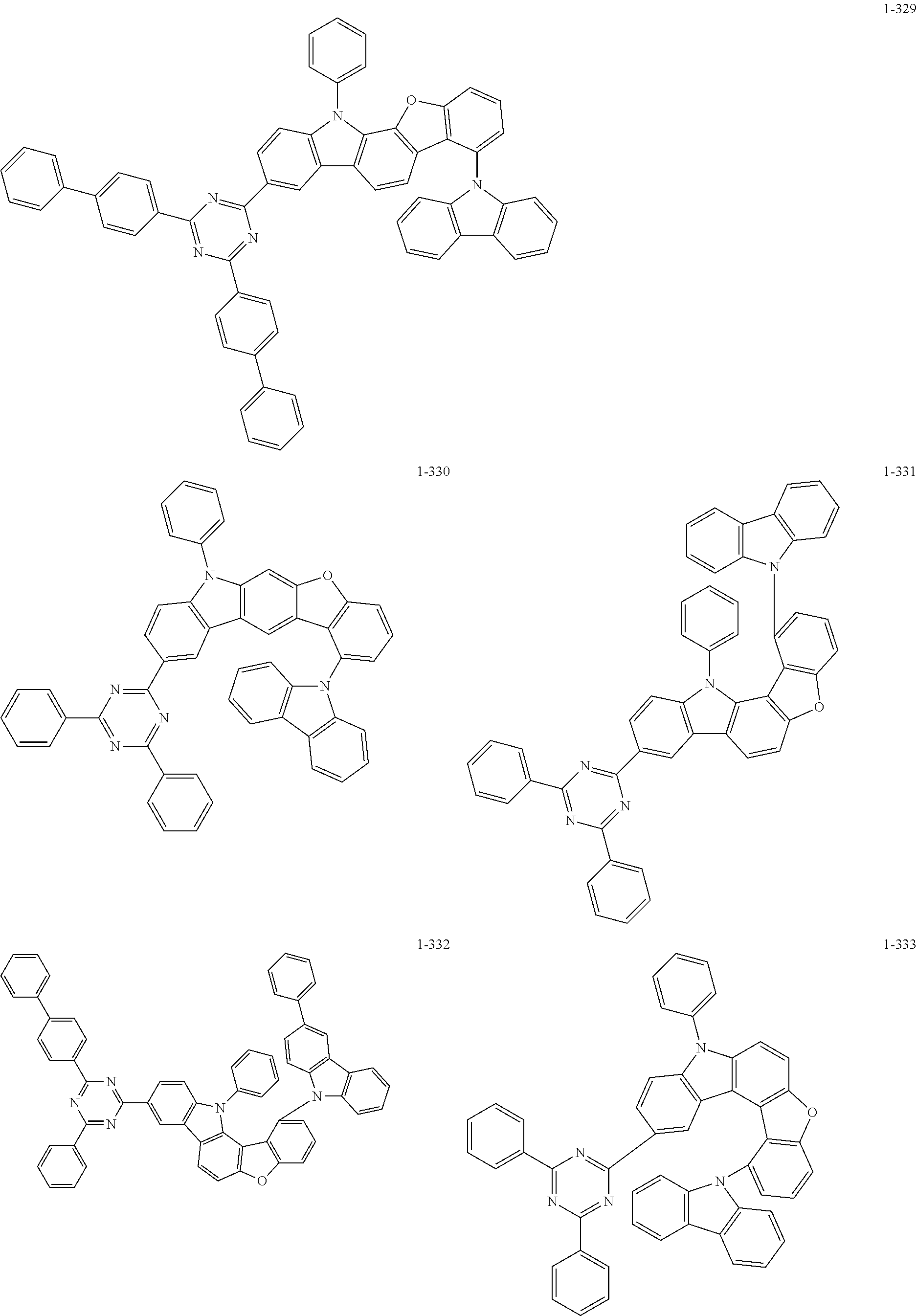

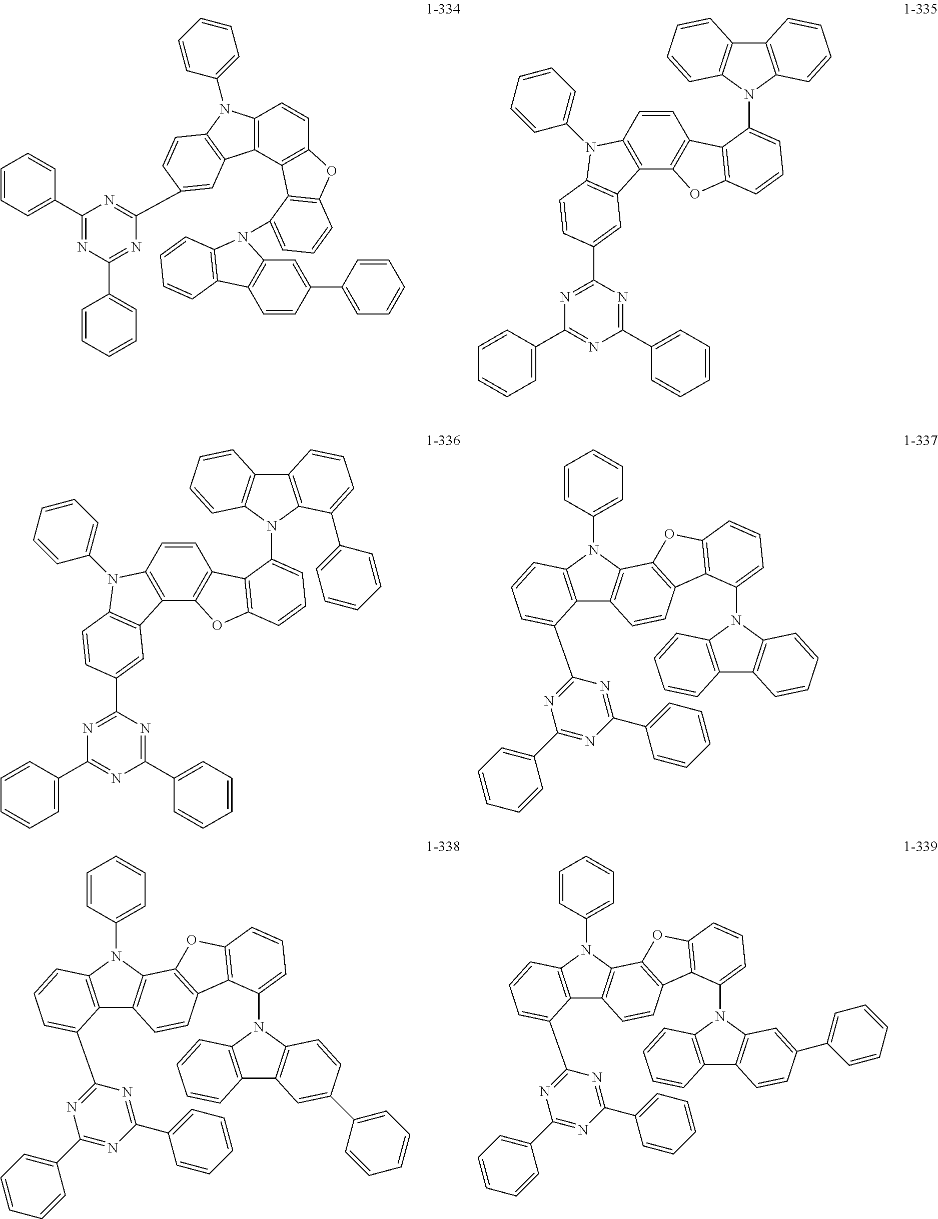

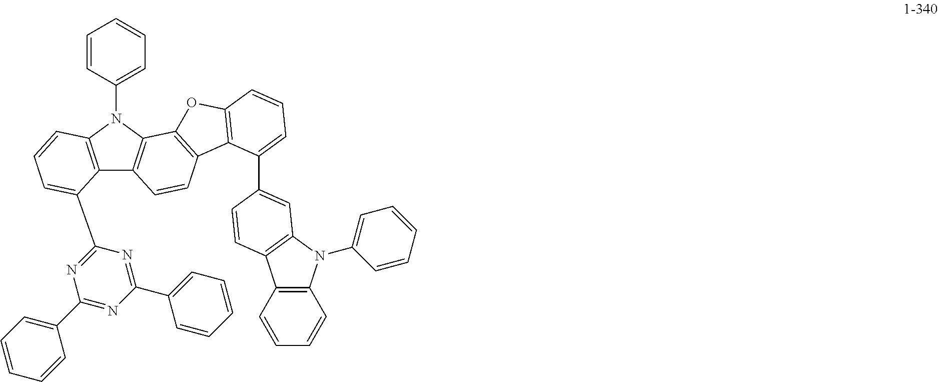 [SMILES: c1ccc(-c2nc(-c3ccccc3)nc(-c3cccc4c3c3ccc5c(oc6cccc(-c7ccc8c9ccccc9n(-c9ccccc9)c8c7)c65)c3n4-c3ccccc3)n2)cc1]

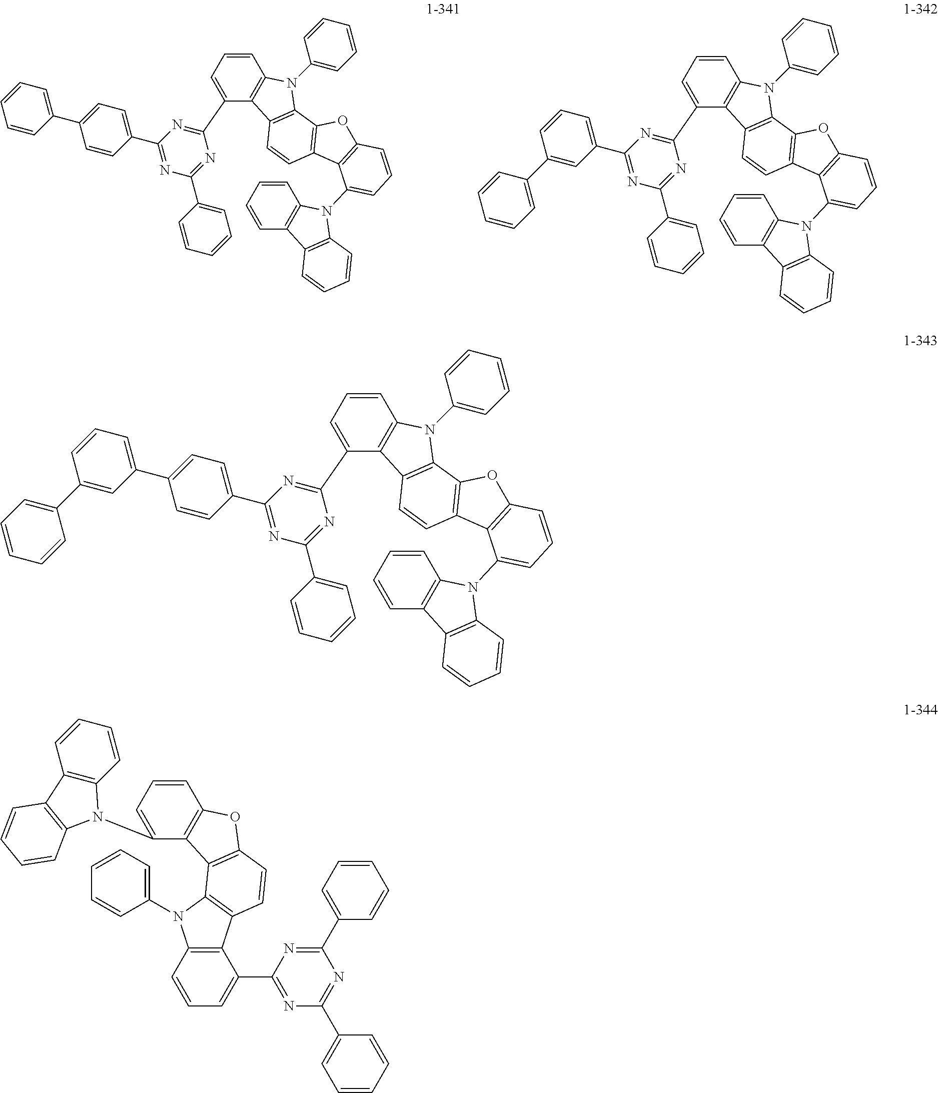 [SMILES: c1ccc(-c2ccc(-c3nc(-c4ccccc4)nc(-c4cccc5c4c4ccc6c(oc7cccc(-n8c9ccccc9c9ccccc98)c76)c4n5-c4ccccc4)n3)cc2)cc1 ; c1ccc(-c2cccc(-c3nc(-c4ccccc4)nc(-c4cccc5c4c4ccc6c(oc7cccc(-n8c9ccccc9c9ccccc98)c76)c4n5-c4ccccc4)n3)c2)cc1 ; c1ccc(-c2cccc(-c3ccc(-c4nc(-c5ccccc5)nc(-c5cccc6c5c5ccc7c(oc8cccc(-n9c%10ccccc%10c%10ccccc%109)c87)c5n6-c5ccccc5)n4)cc3)c2)cc1 ; c1ccc(-c2nc(-c3ccccc3)nc(-c3cccc4c3c3ccc5oc6cccc(-n7c8ccccc8c8ccccc87)c6c5c3n4-c3ccccc3)n2)cc1]

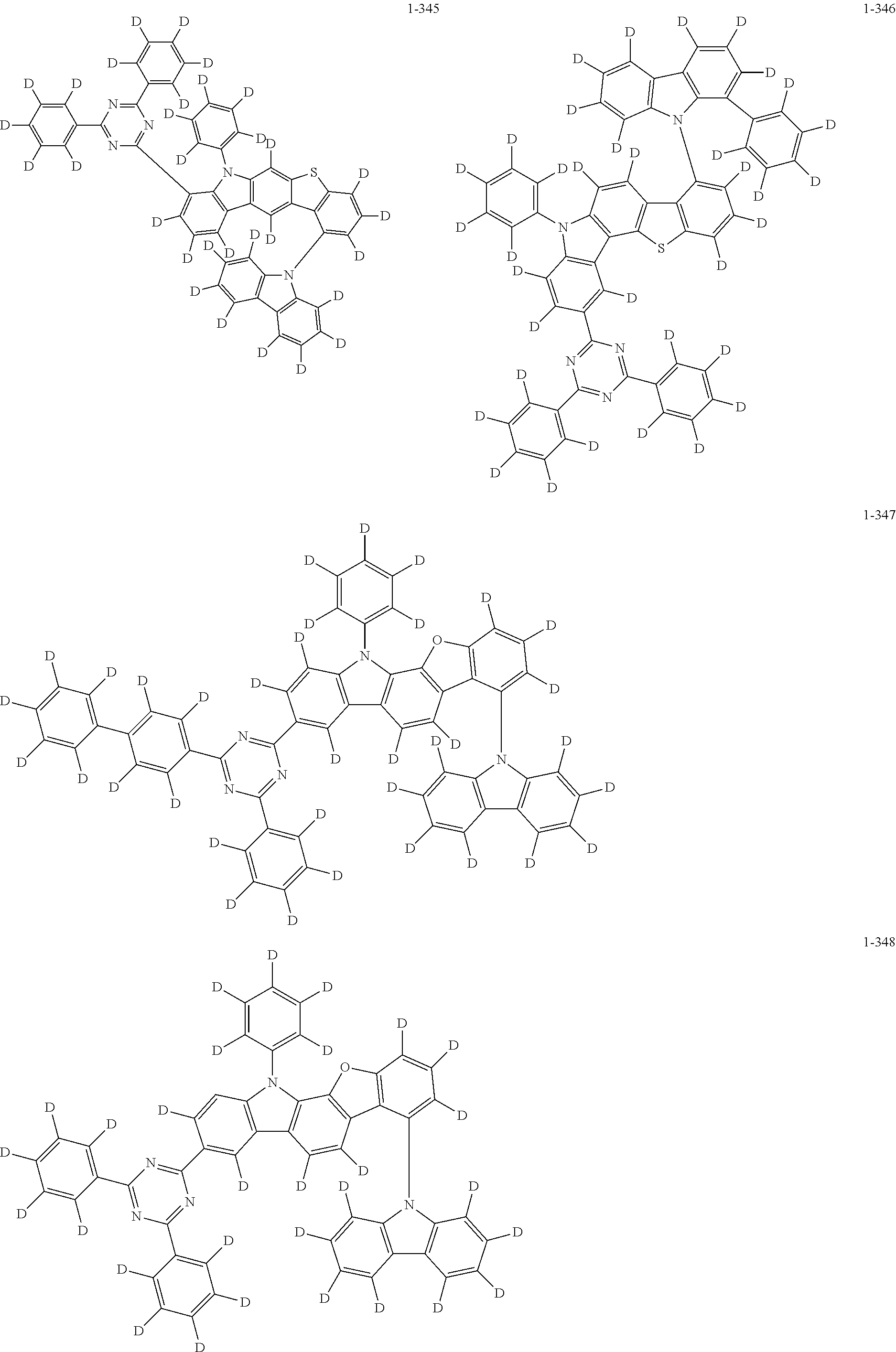

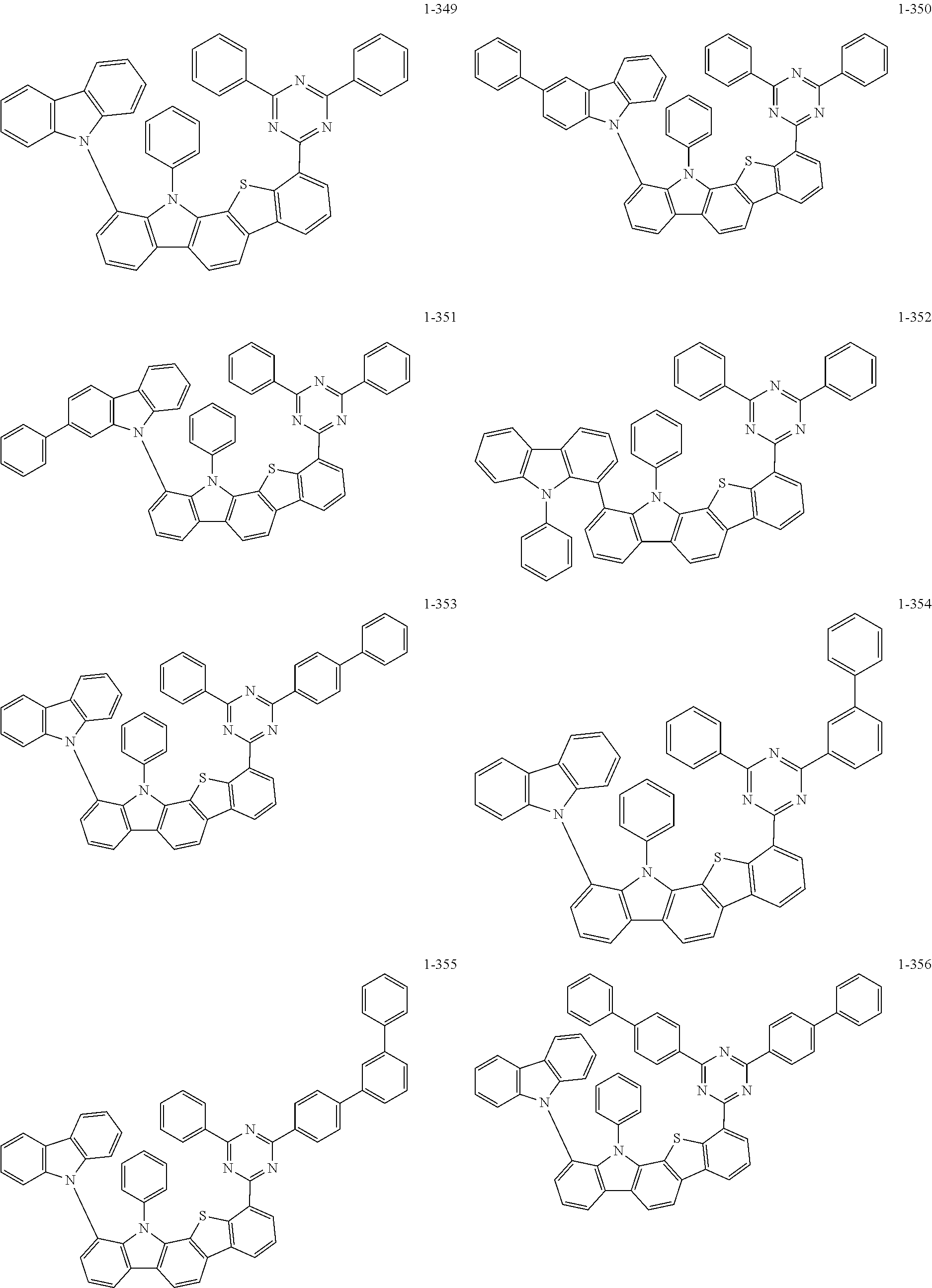

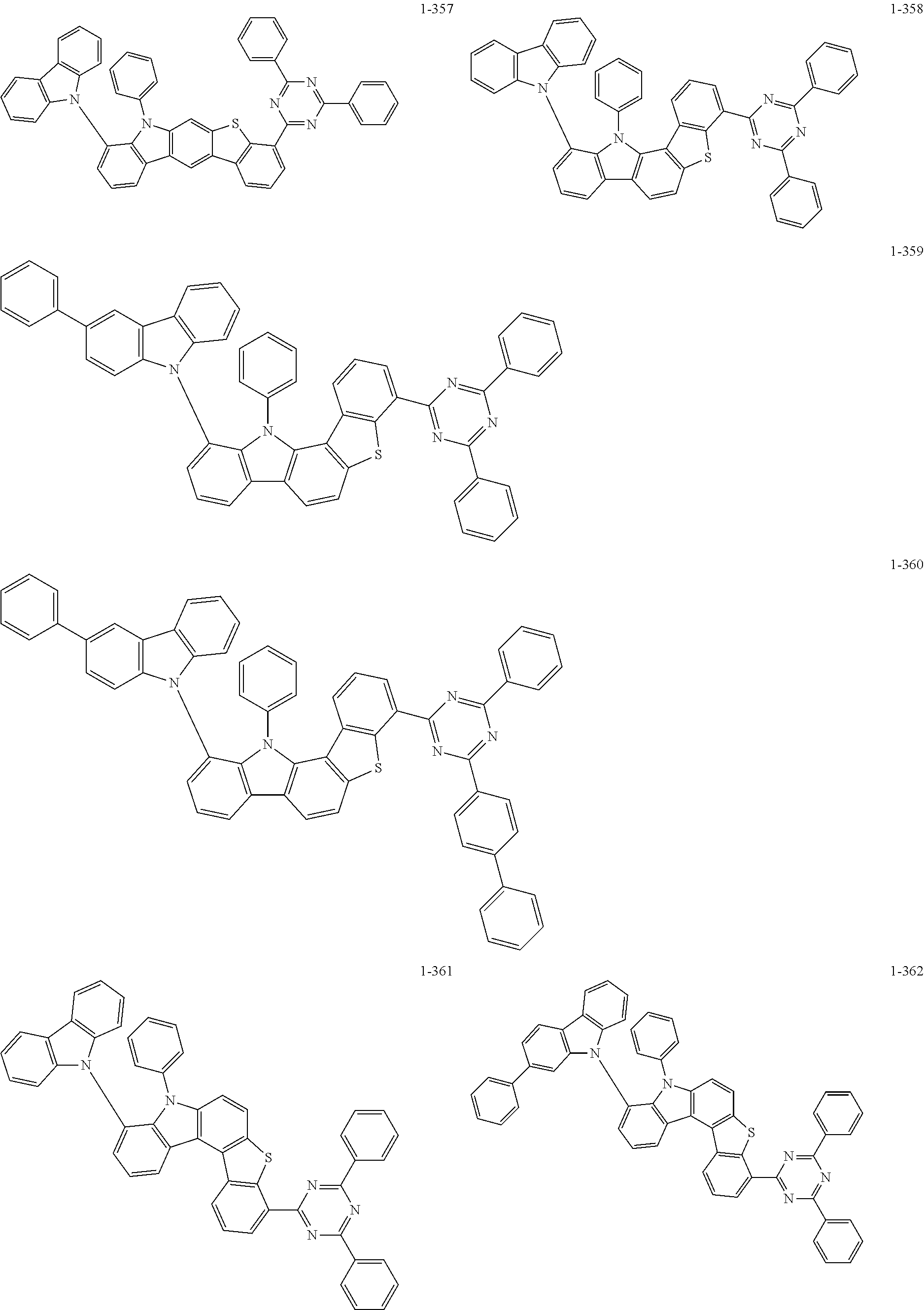

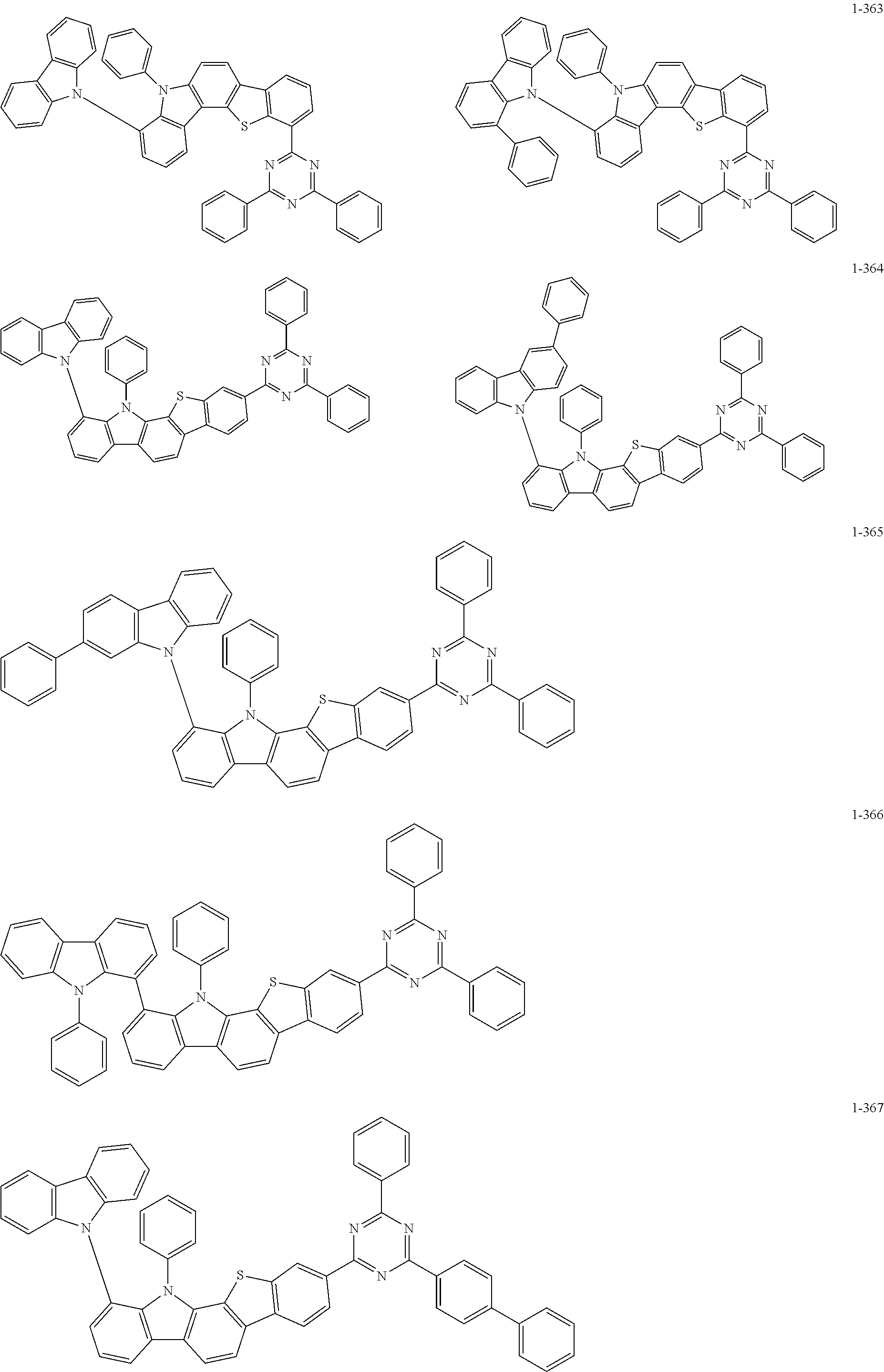

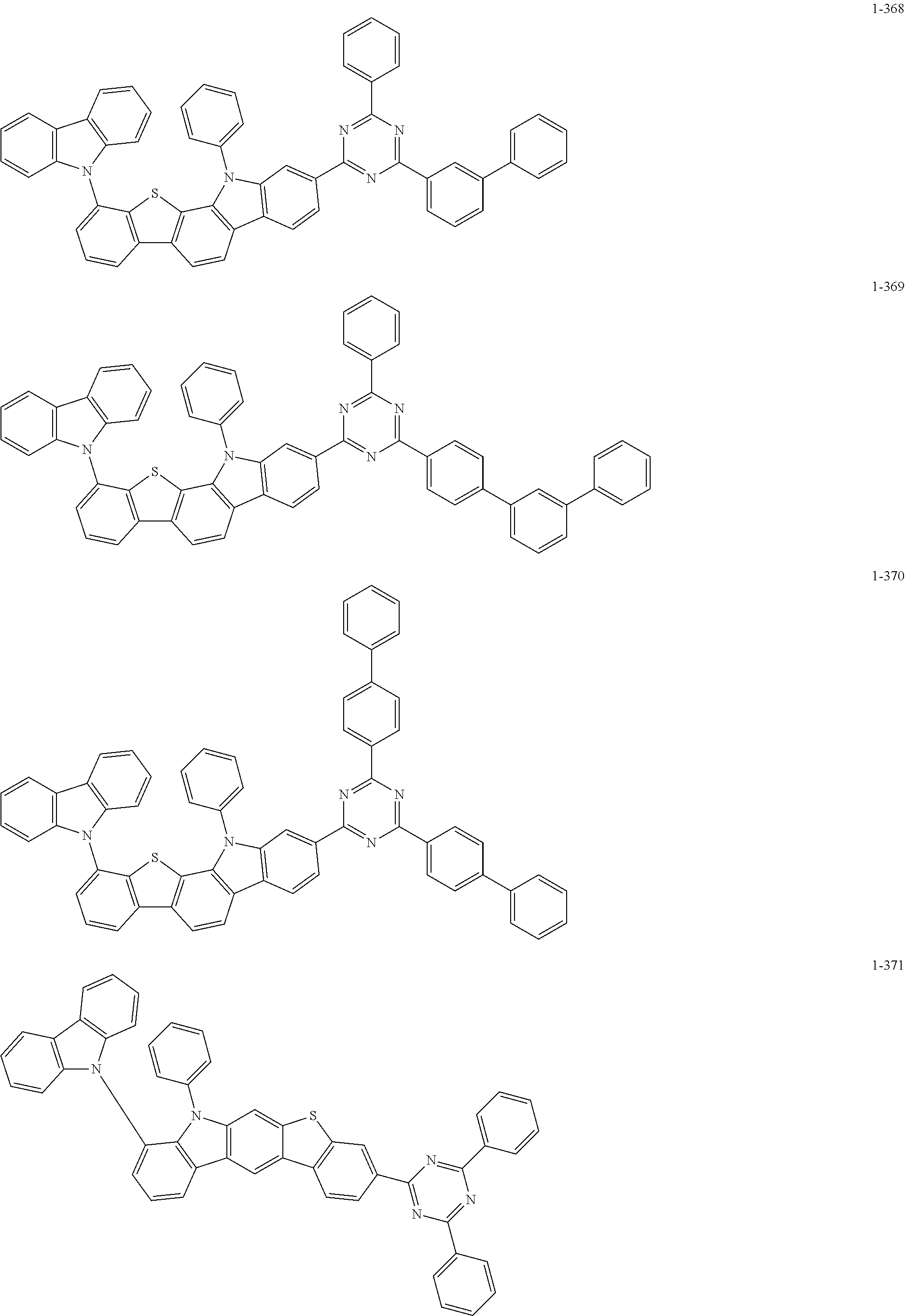

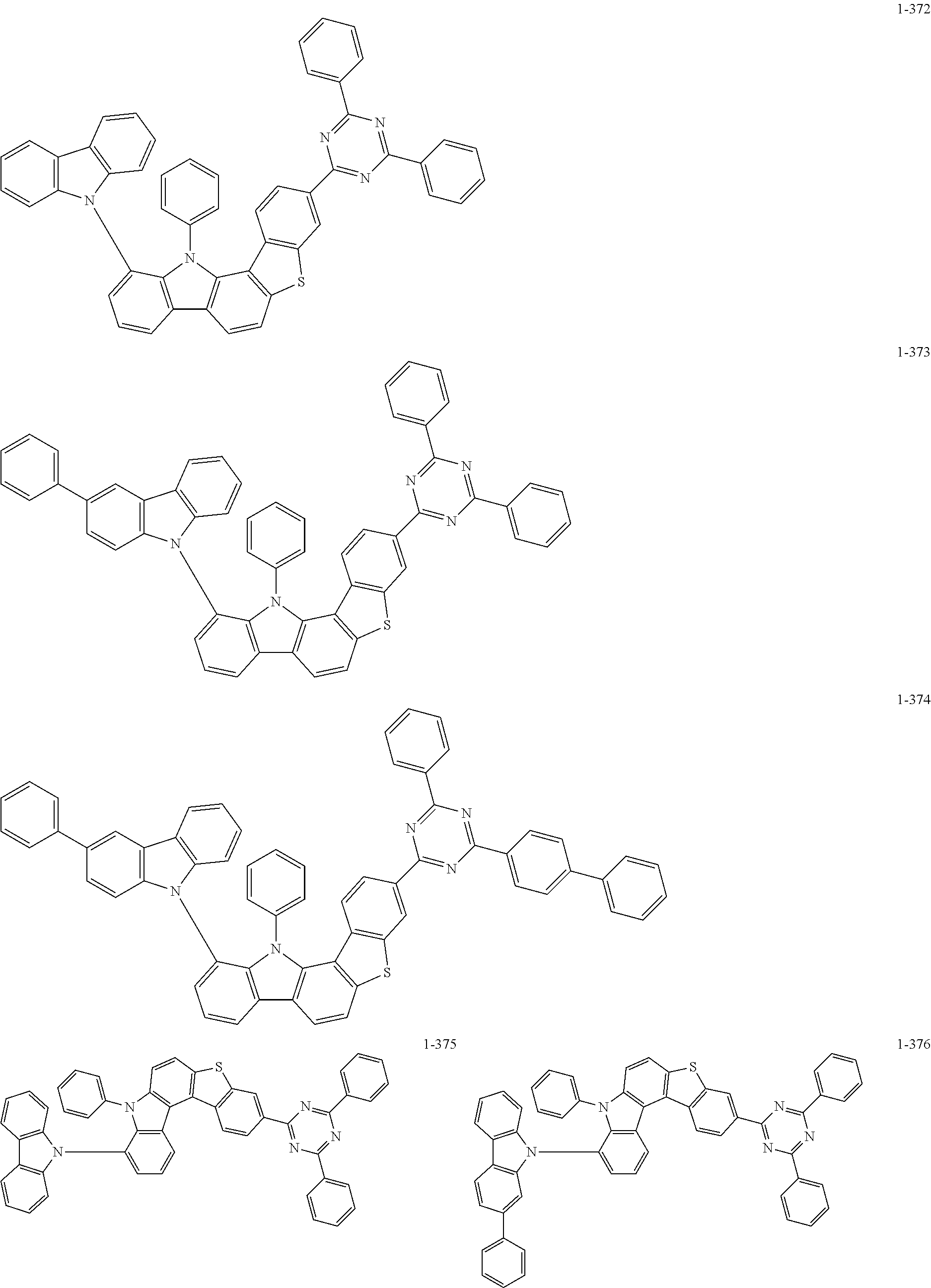

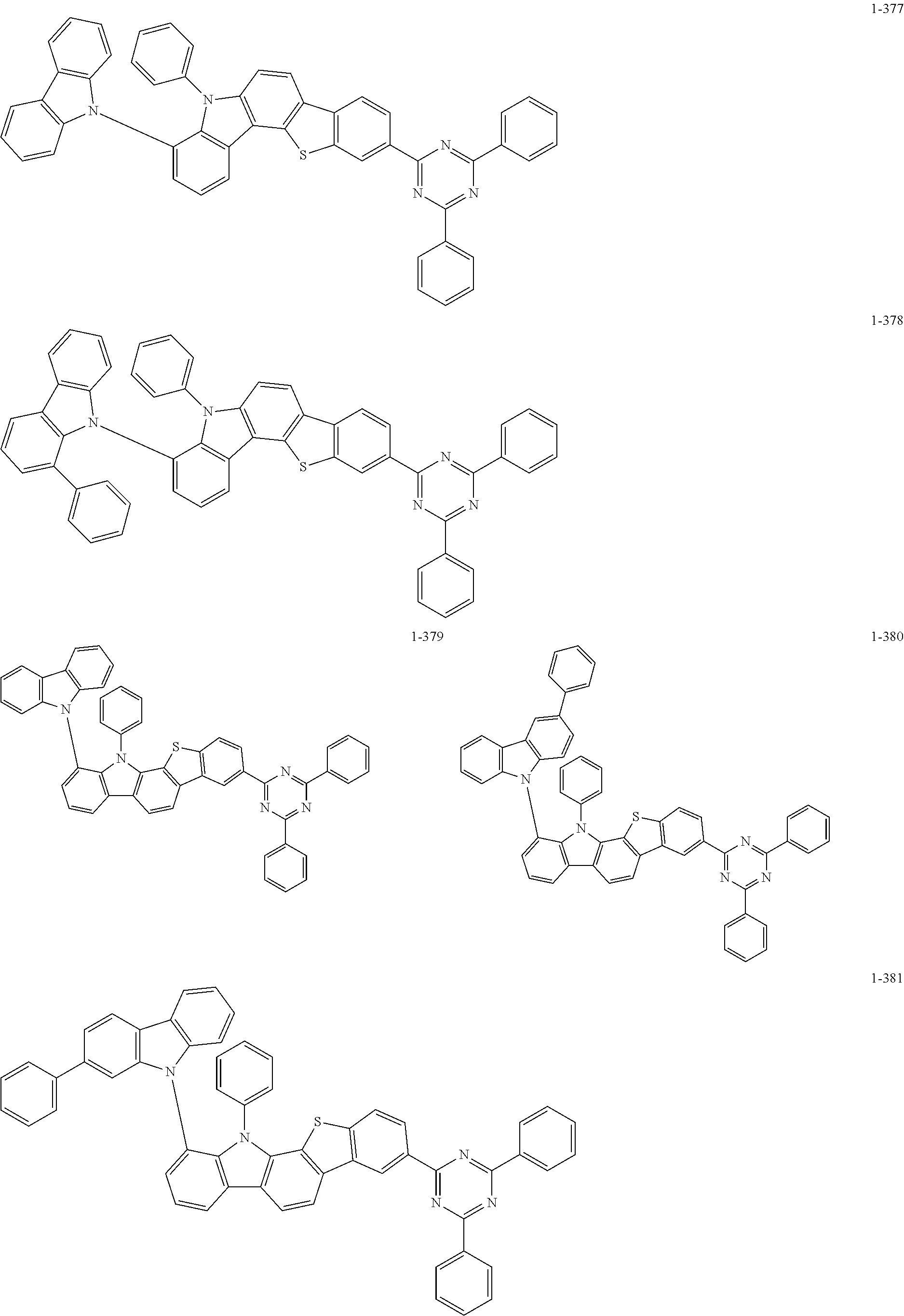

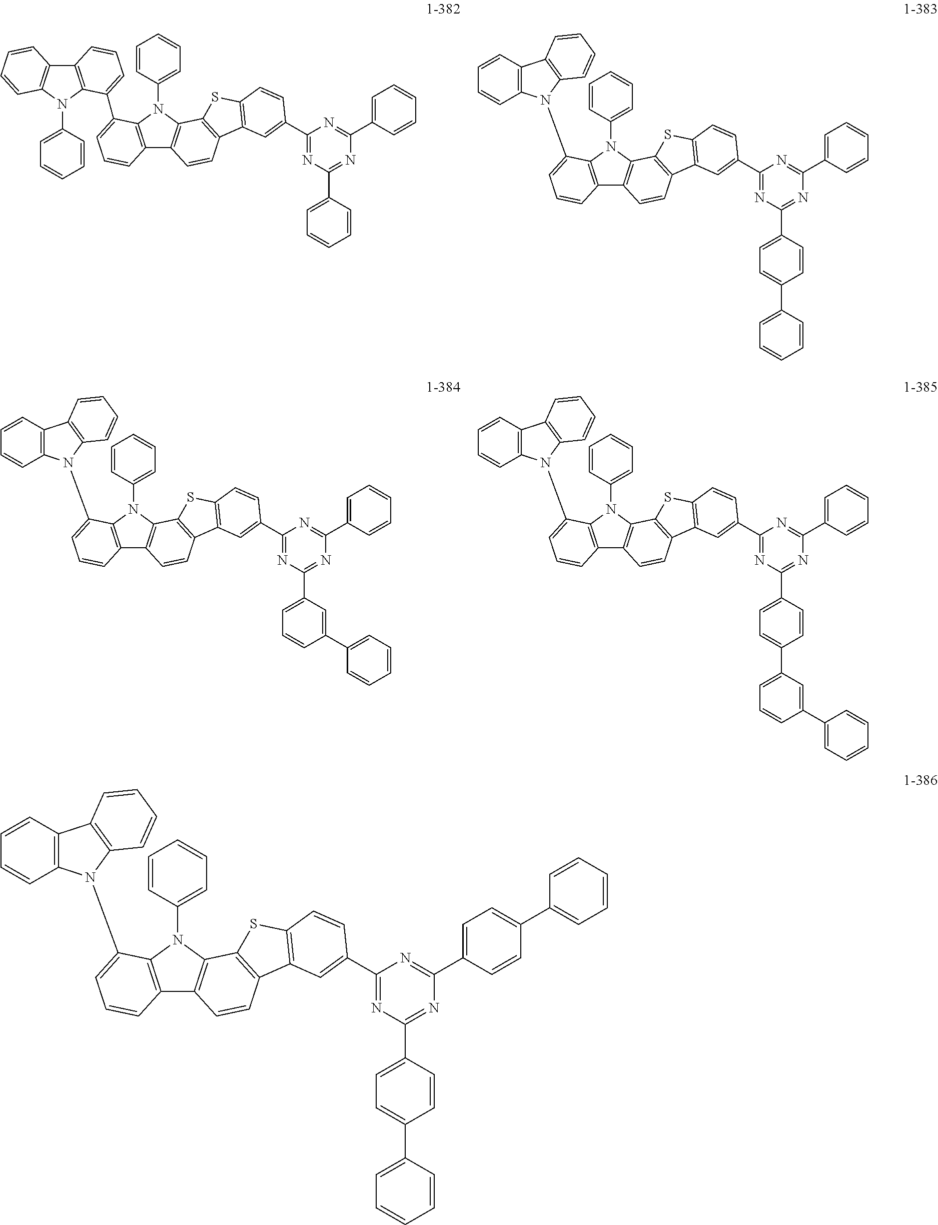

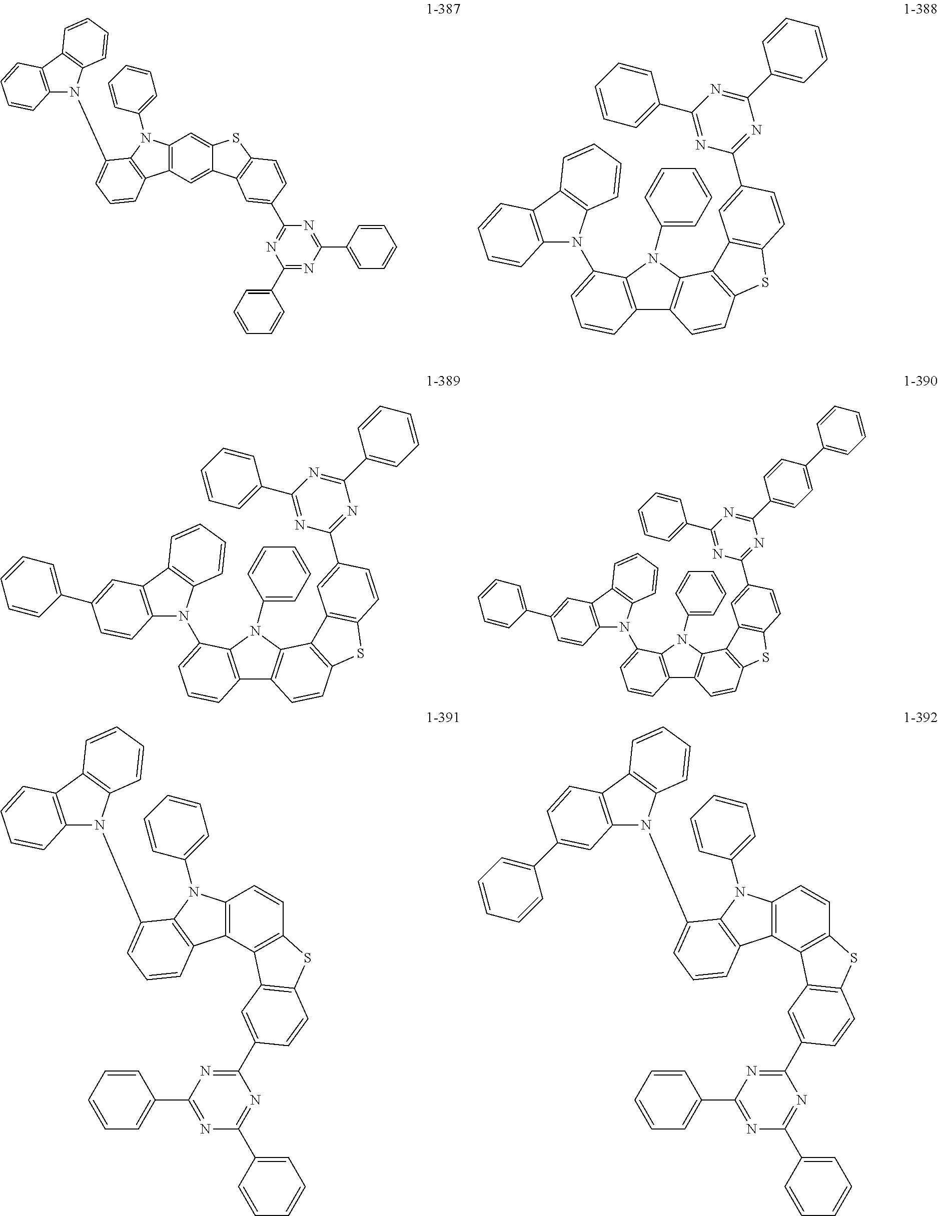

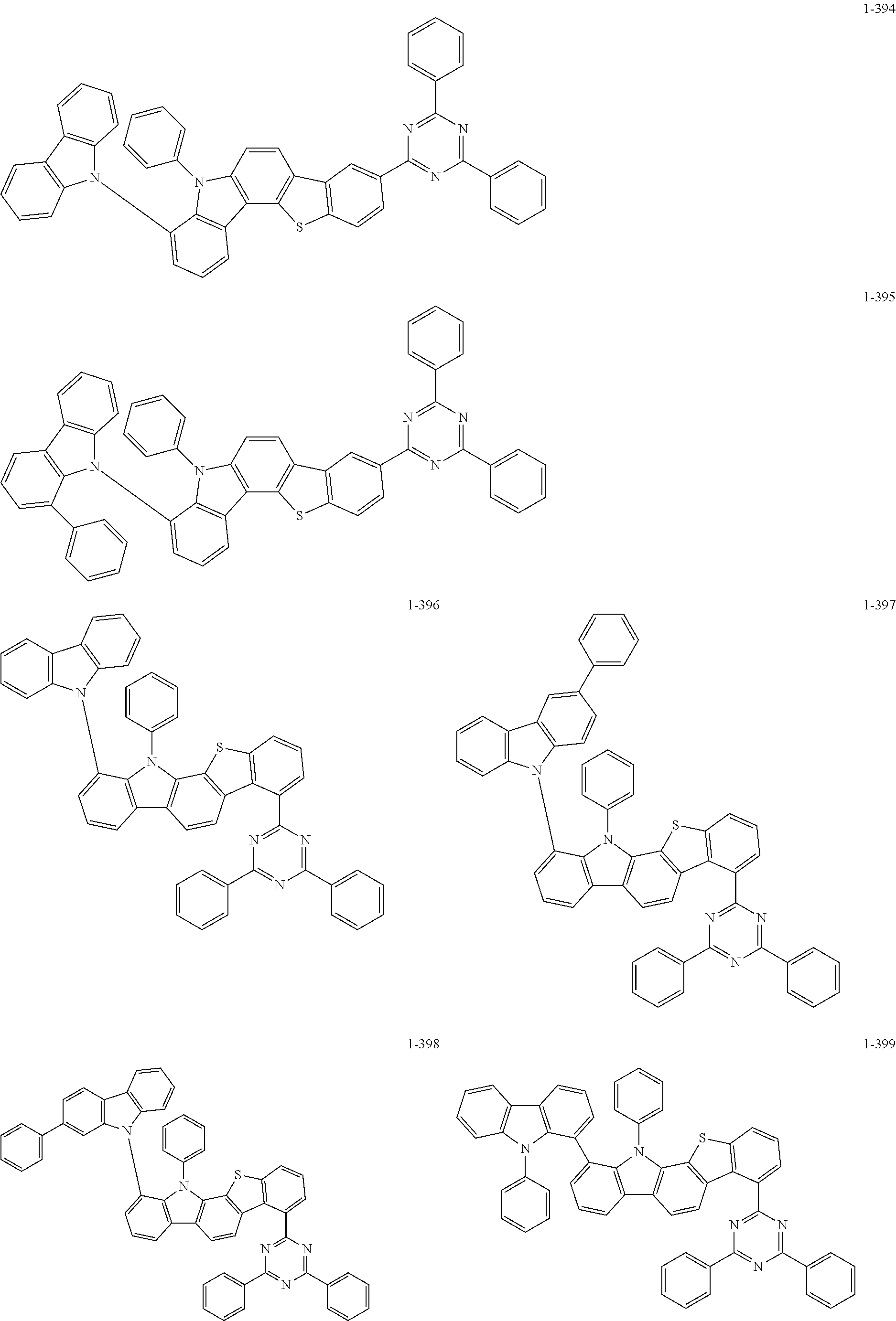

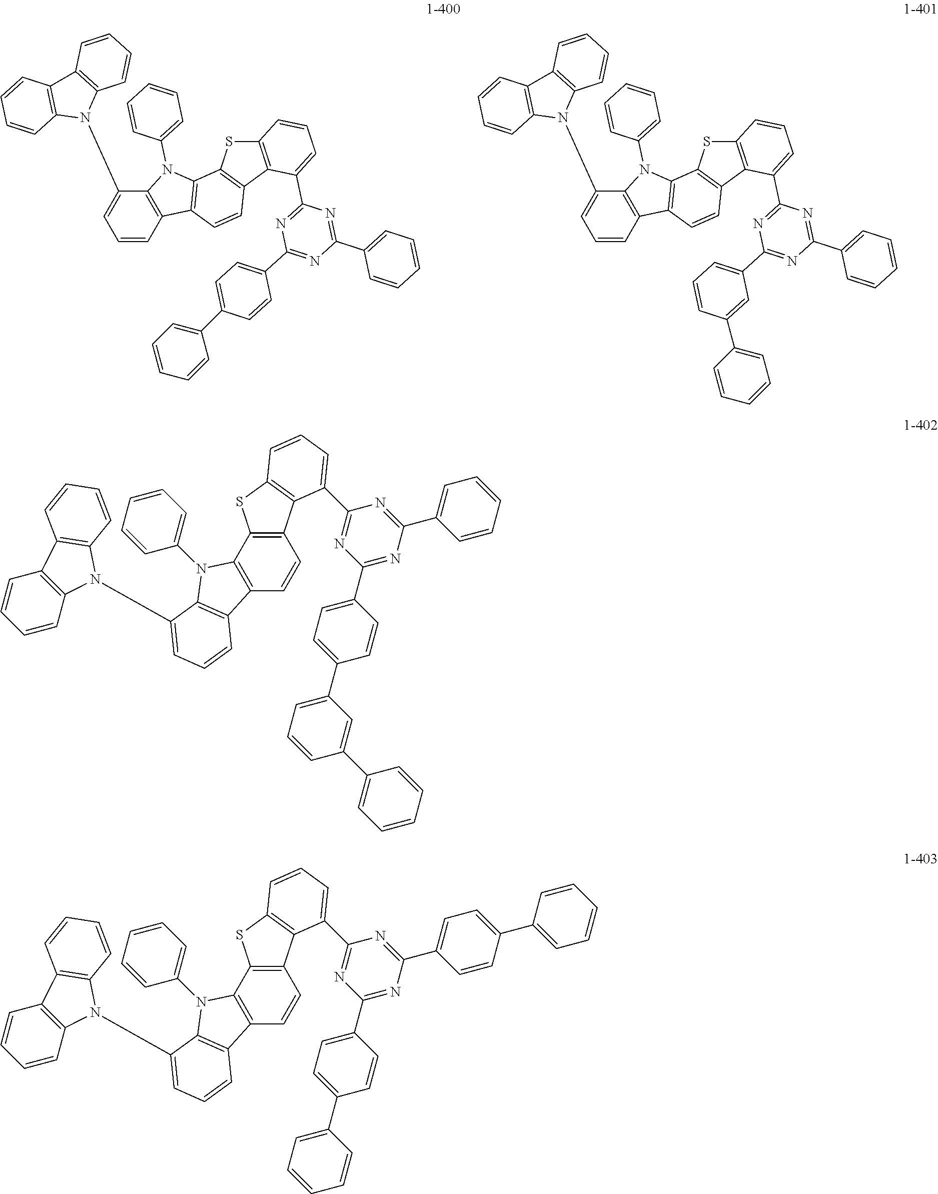

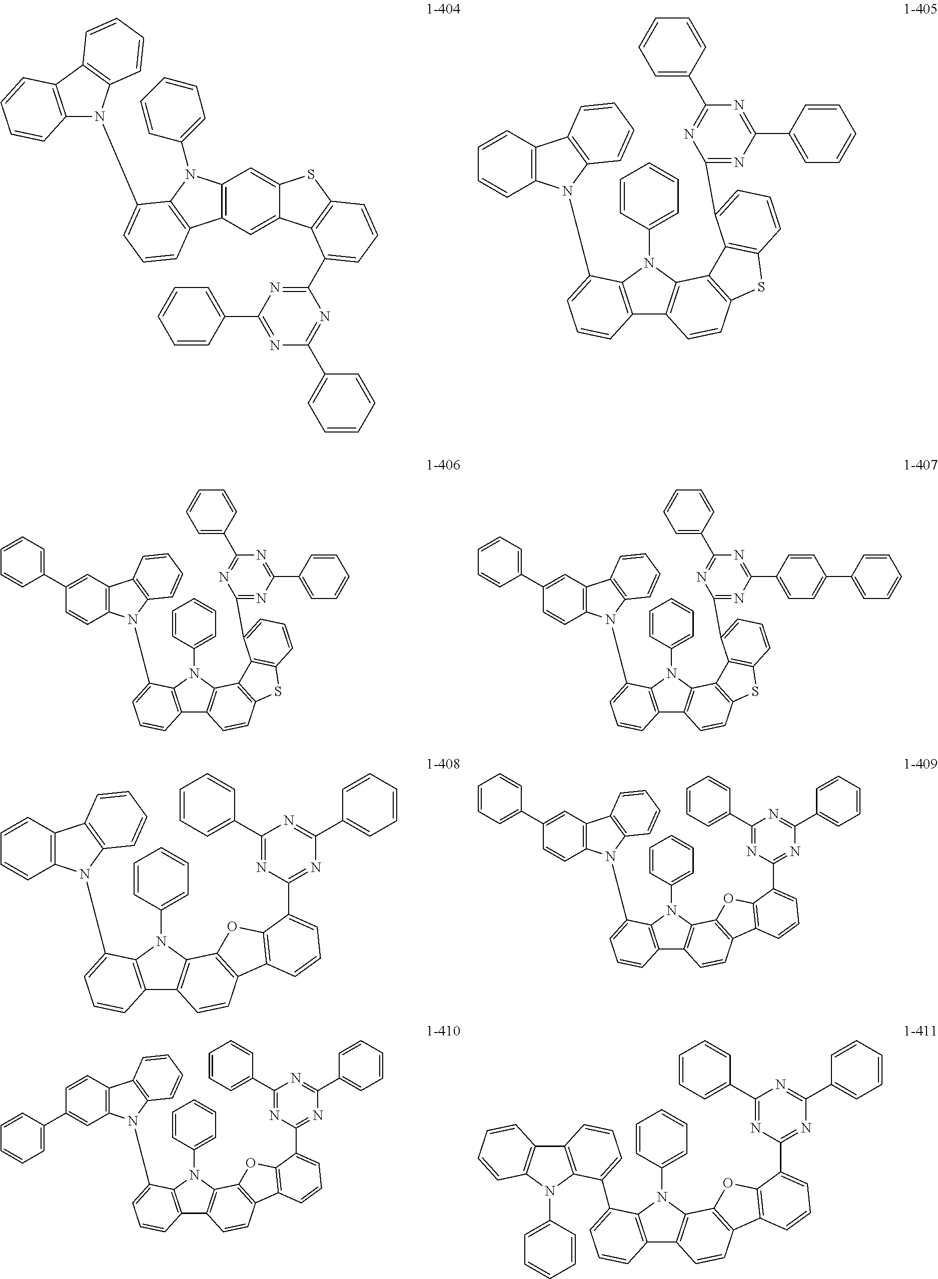

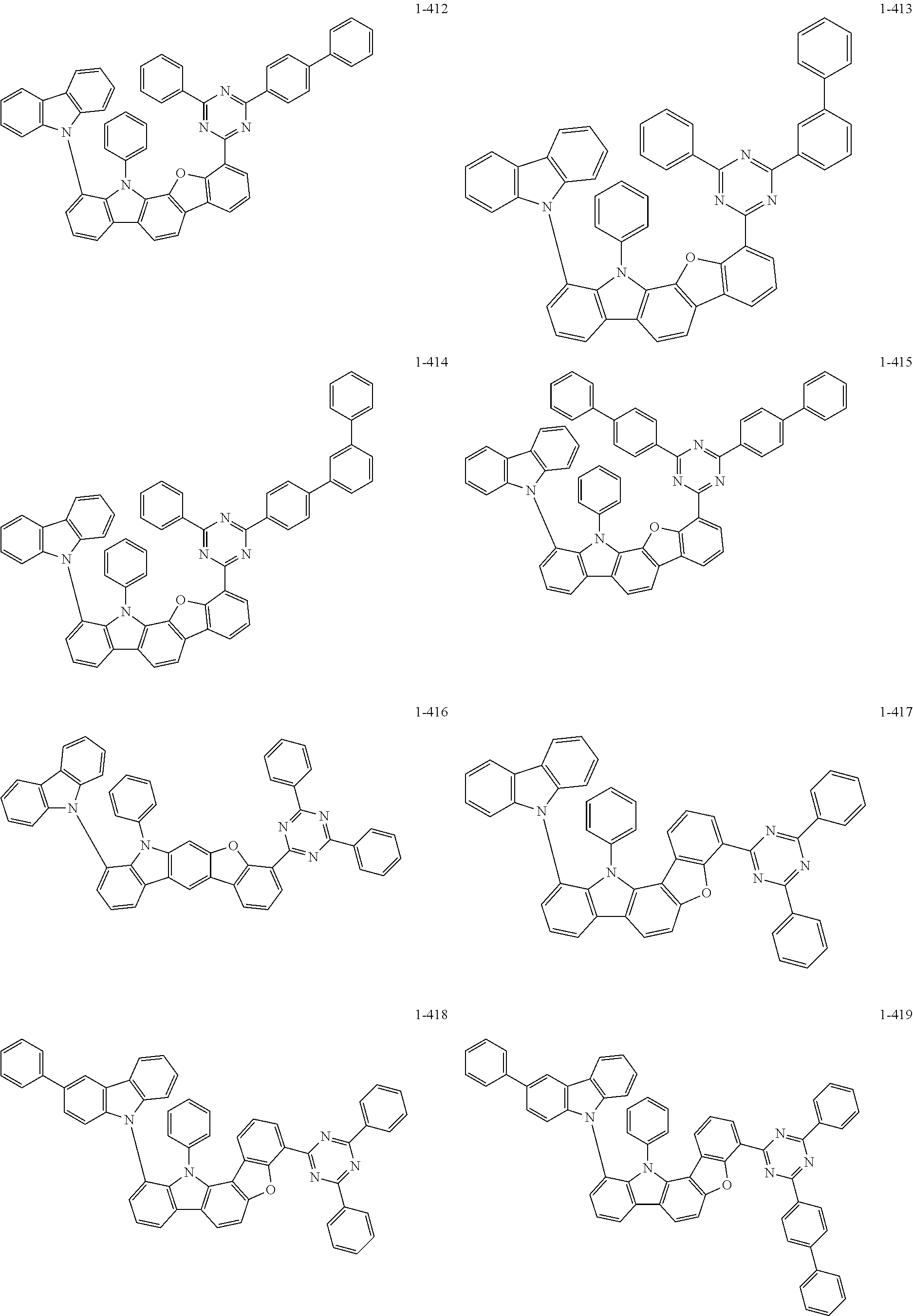

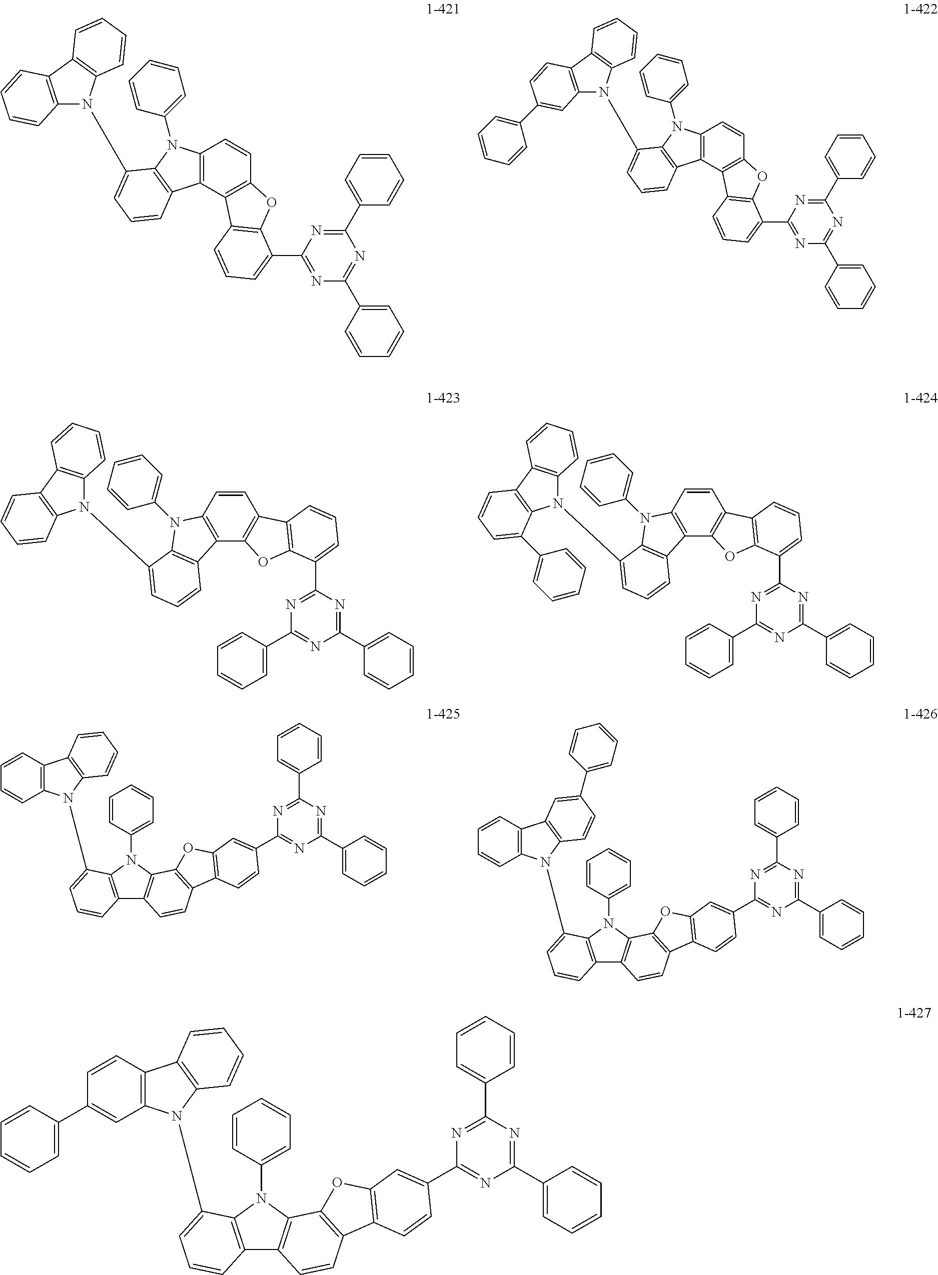

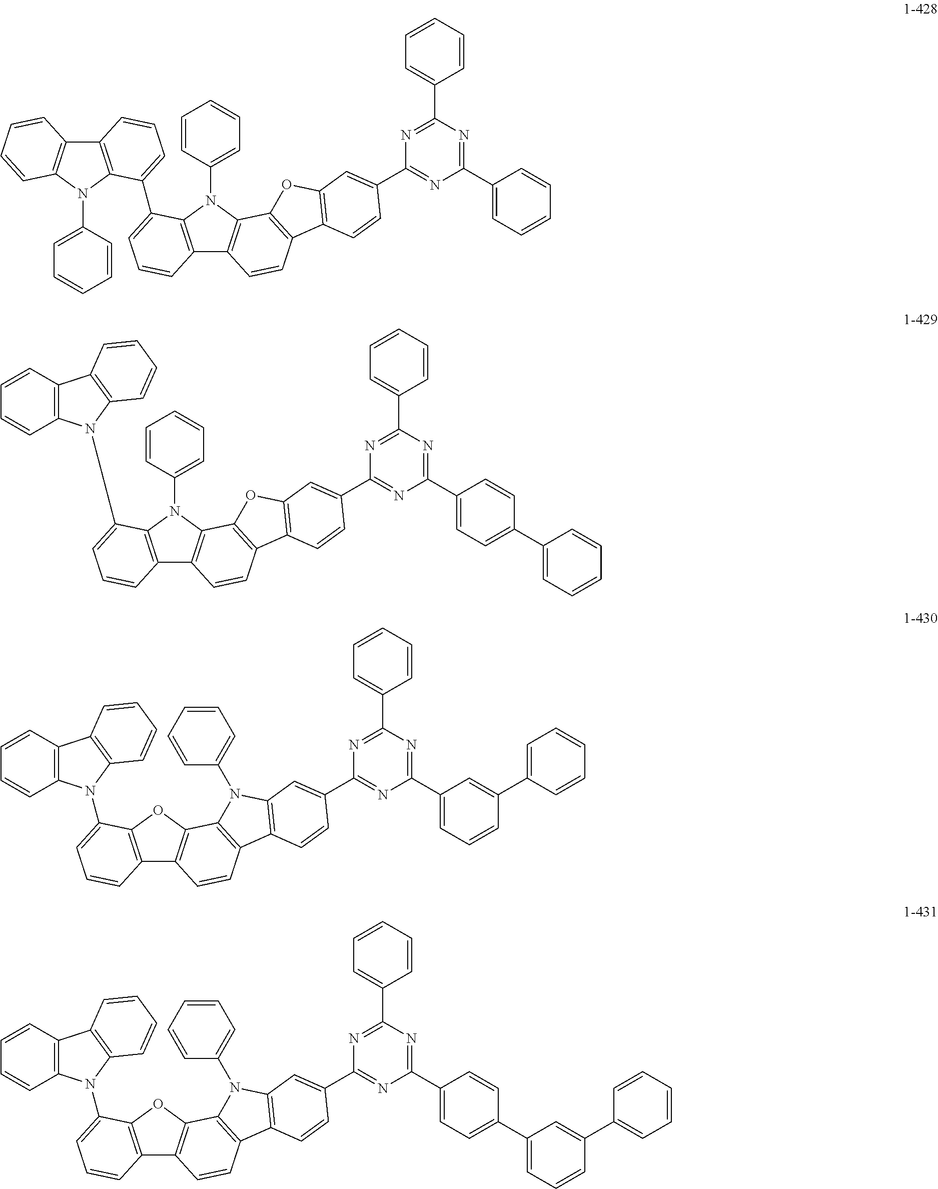 [SMILES: c1ccc(-c2nc(-c3ccccc3)nc(-c3ccc4c(c3)oc3c4ccc4c5cccc(-c6cccc7c8ccccc8n(-c8ccccc8)c67)c5n(-c5ccccc5)c43)n2)cc1 ; c1ccc(-c2ccc(-c3nc(-c4ccccc4)nc(-c4ccc5c(c4)oc4c5ccc5c6cccc(-n7c8ccccc8c8ccccc87)c6n(-c6ccccc6)c54)n3)cc2)cc1 ; c1ccc(-c2cccc(-c3nc(-c4ccccc4)nc(-c4ccc5c6ccc7c8cccc(-n9c%10ccccc%10c%10ccccc%109)c8oc7c6n(-c6ccccc6)c5c4)n3)c2)cc1 ; c1ccc(-c2cccc(-c3ccc(-c4nc(-c5ccccc5)nc(-c5ccc6c7ccc8c9cccc(-n%10c%11ccccc%11c%11ccccc%11%10)c9oc8c7n(-c7ccccc7)c6c5)n4)cc3)c2)cc1]

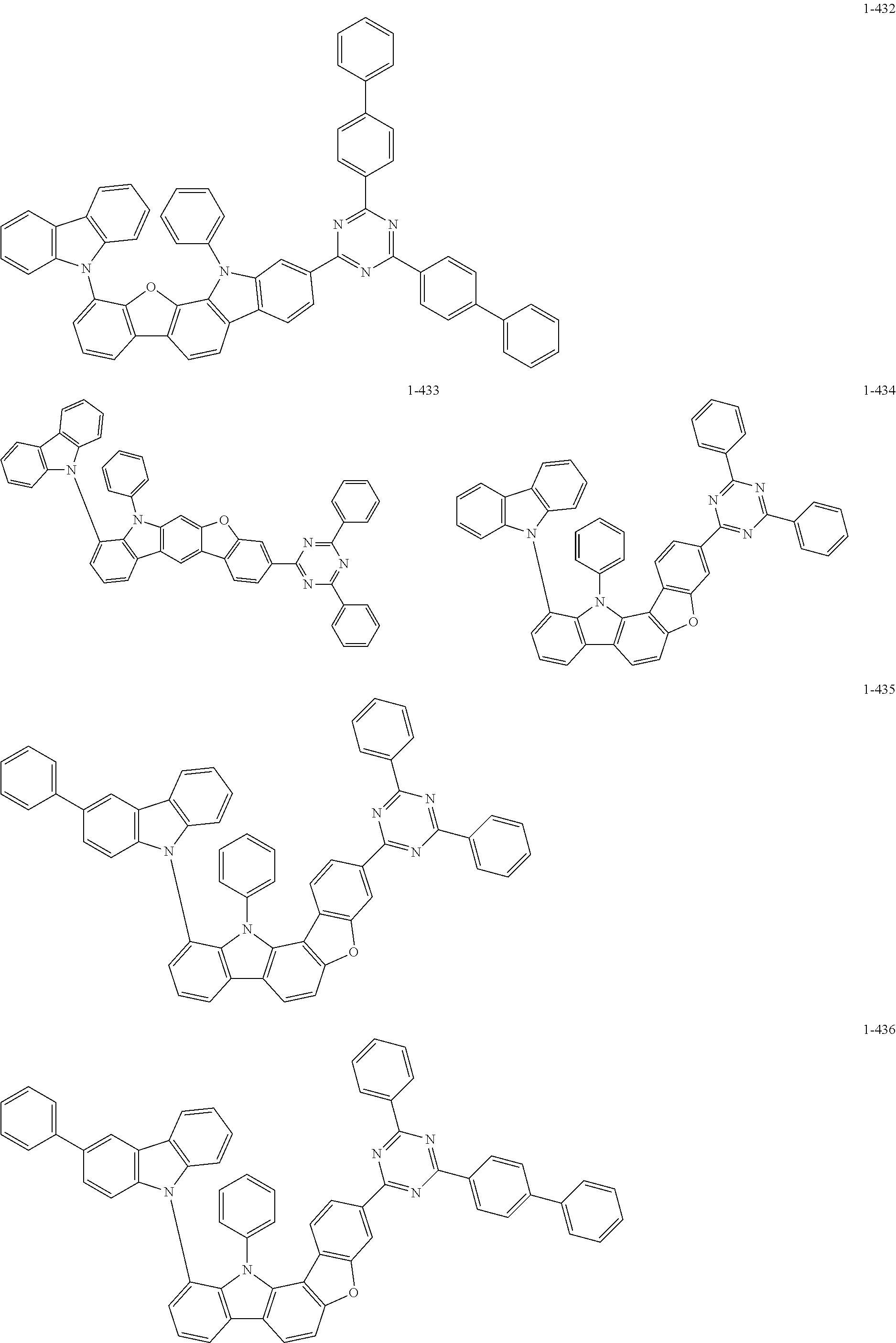

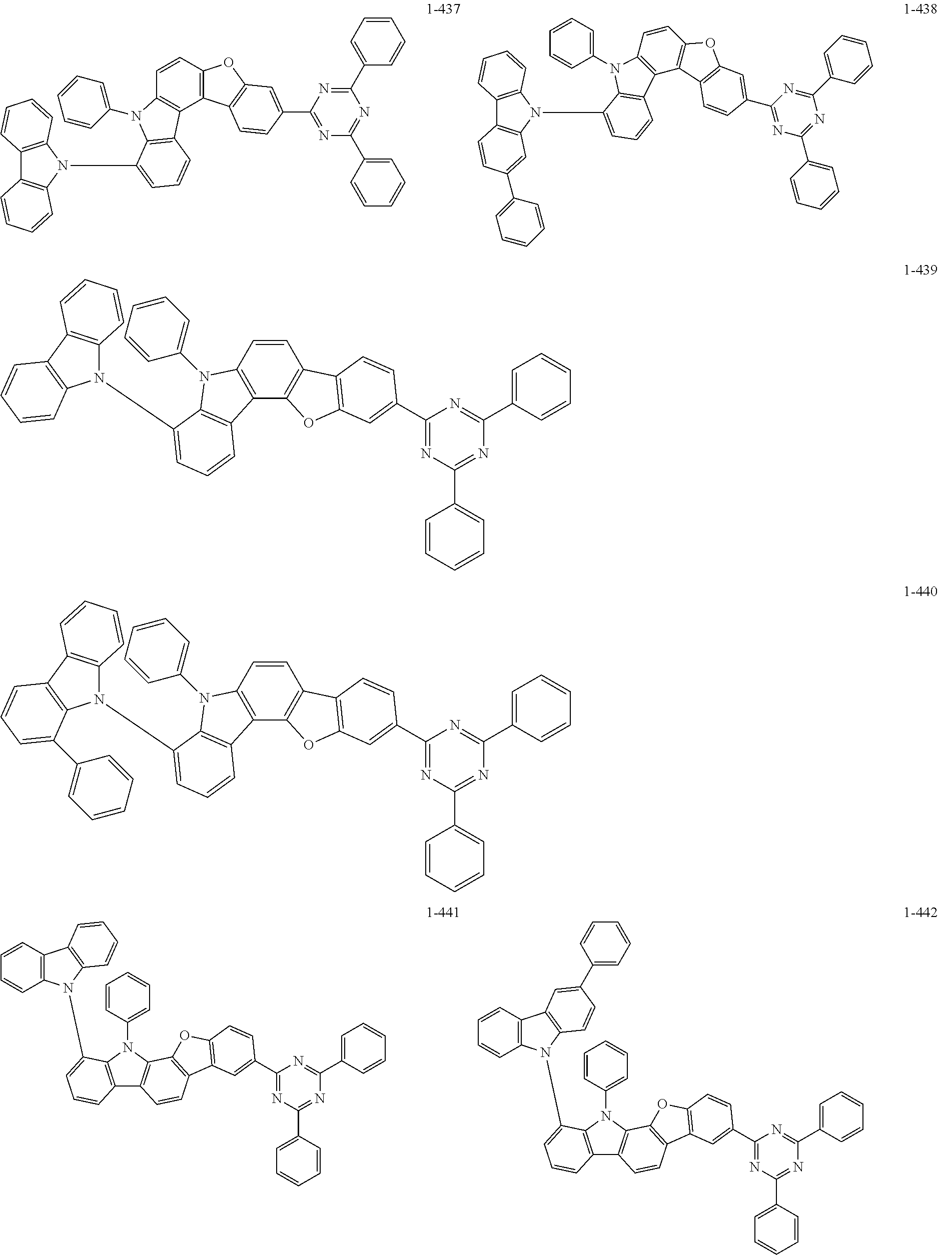

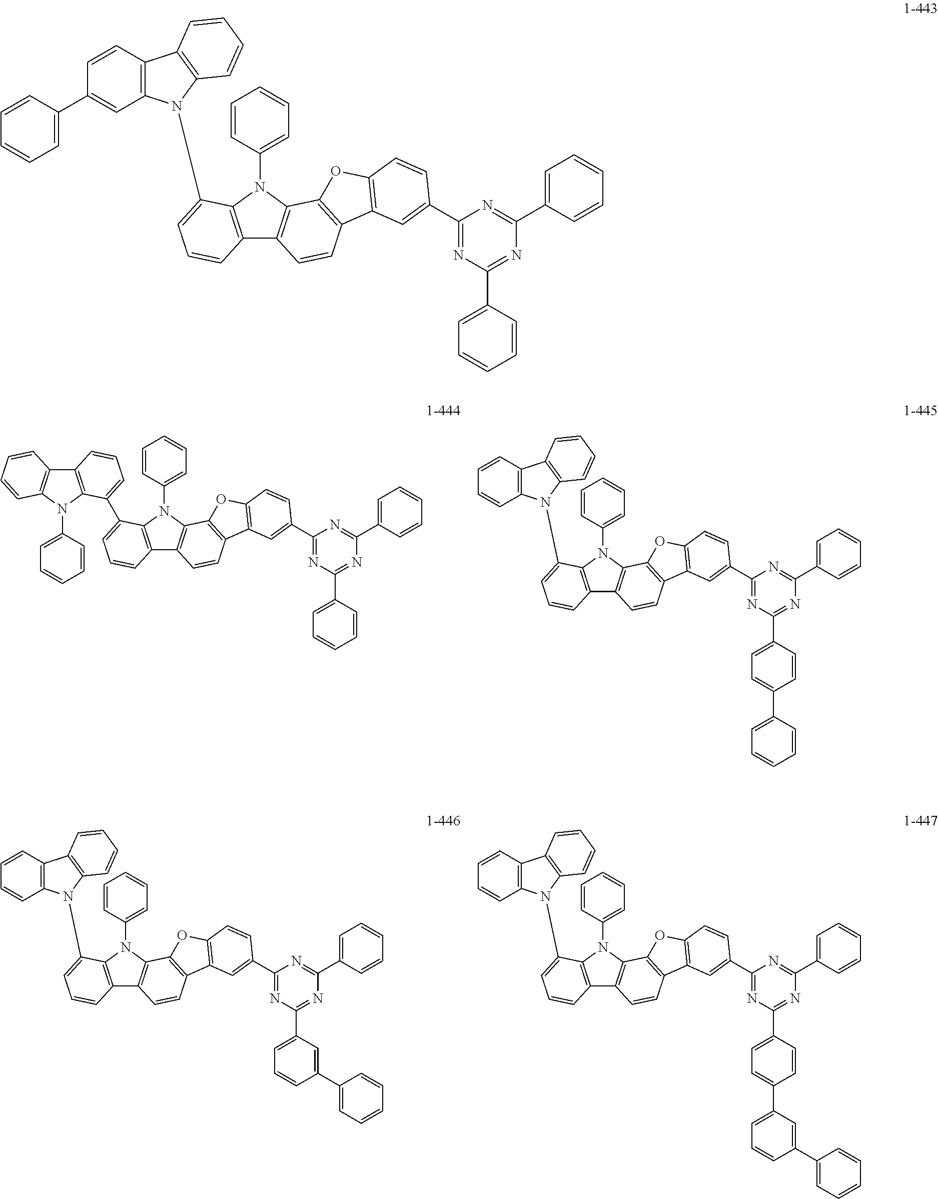

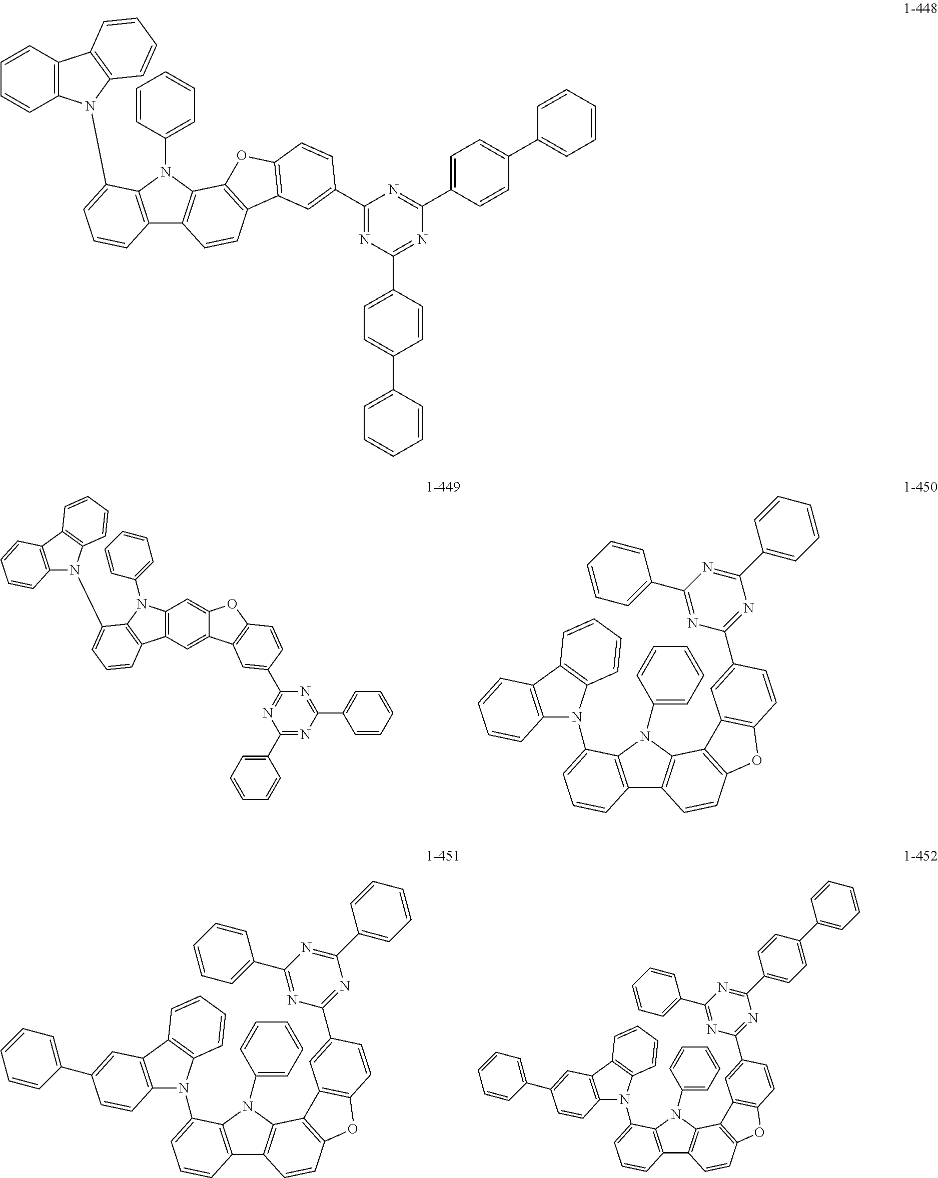

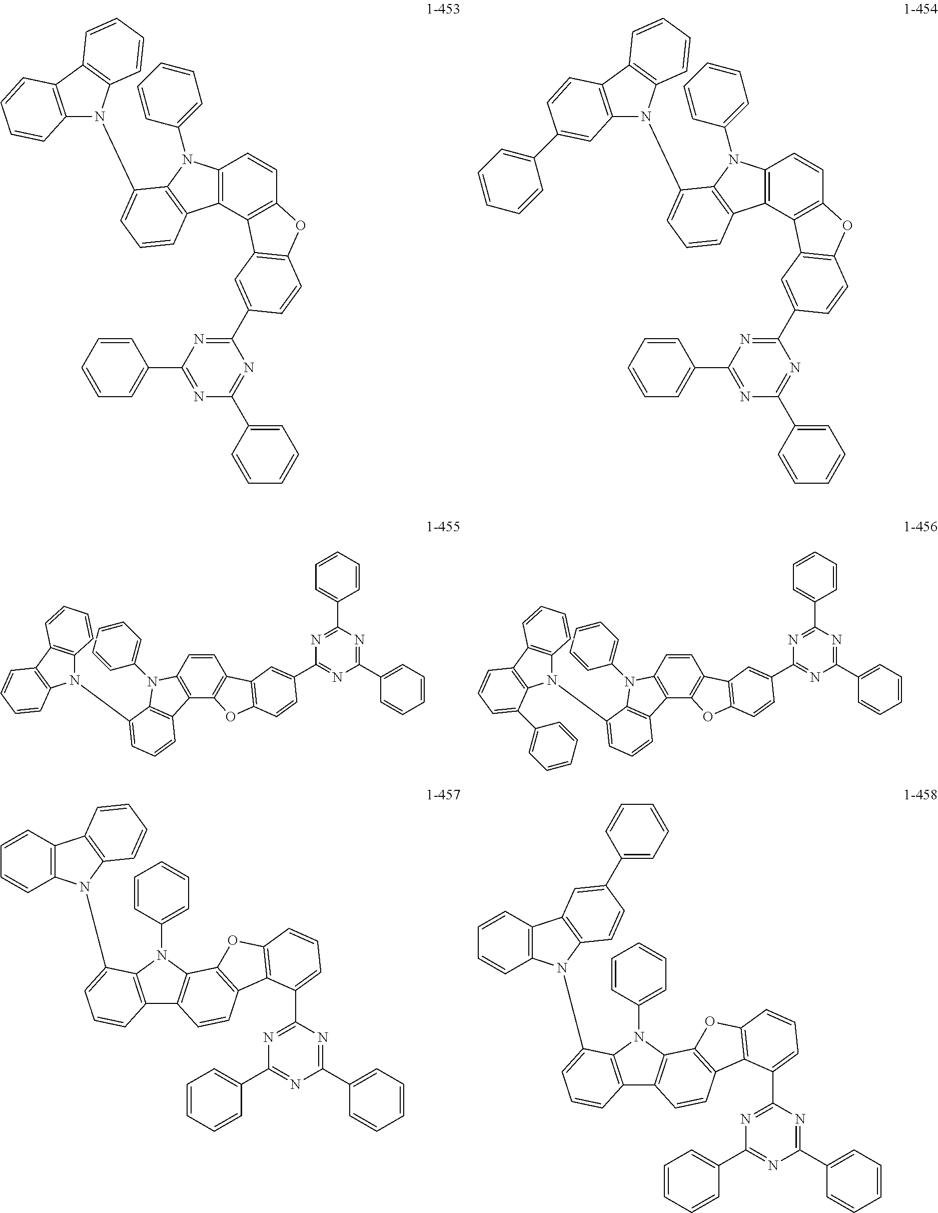

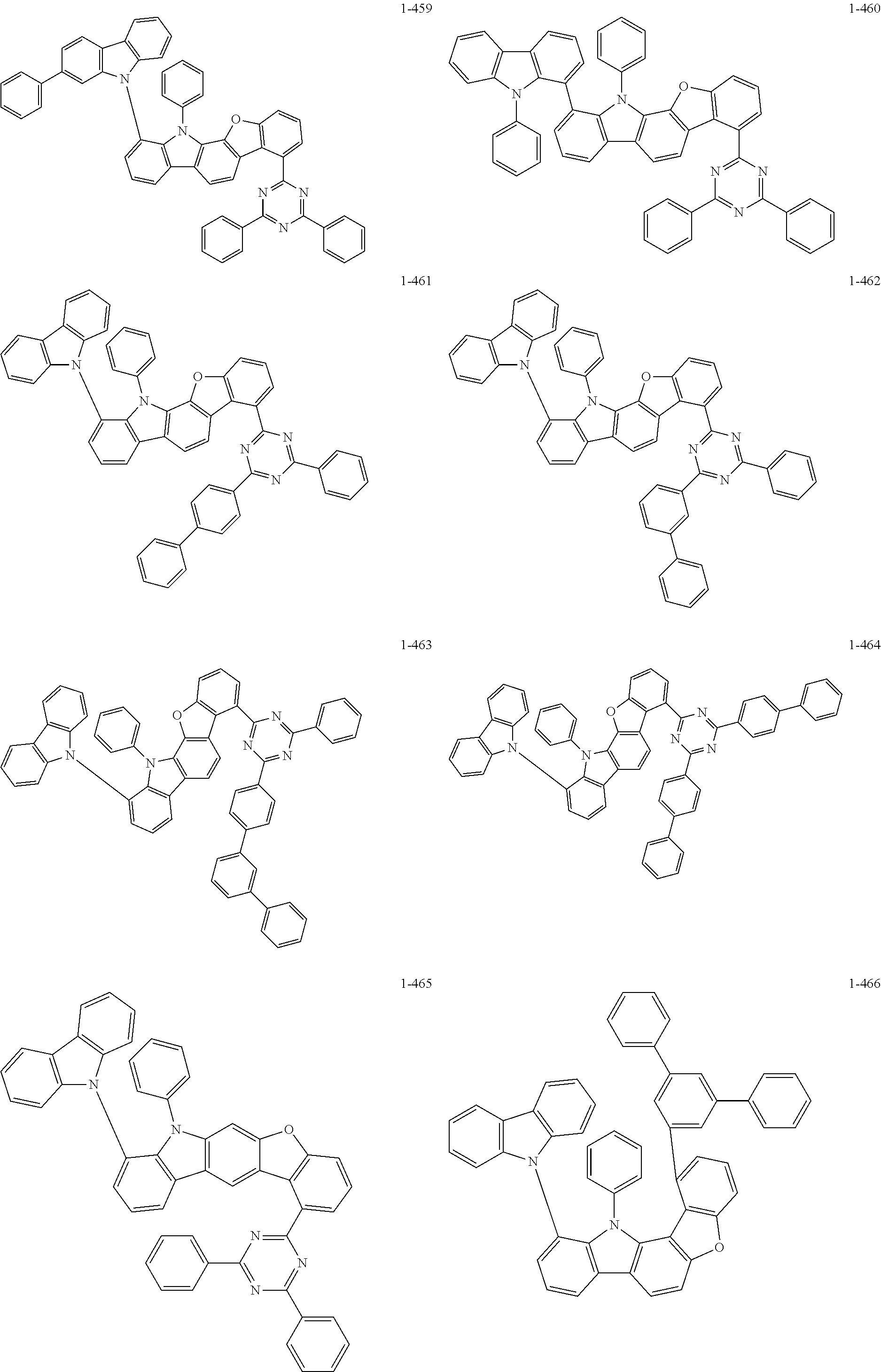

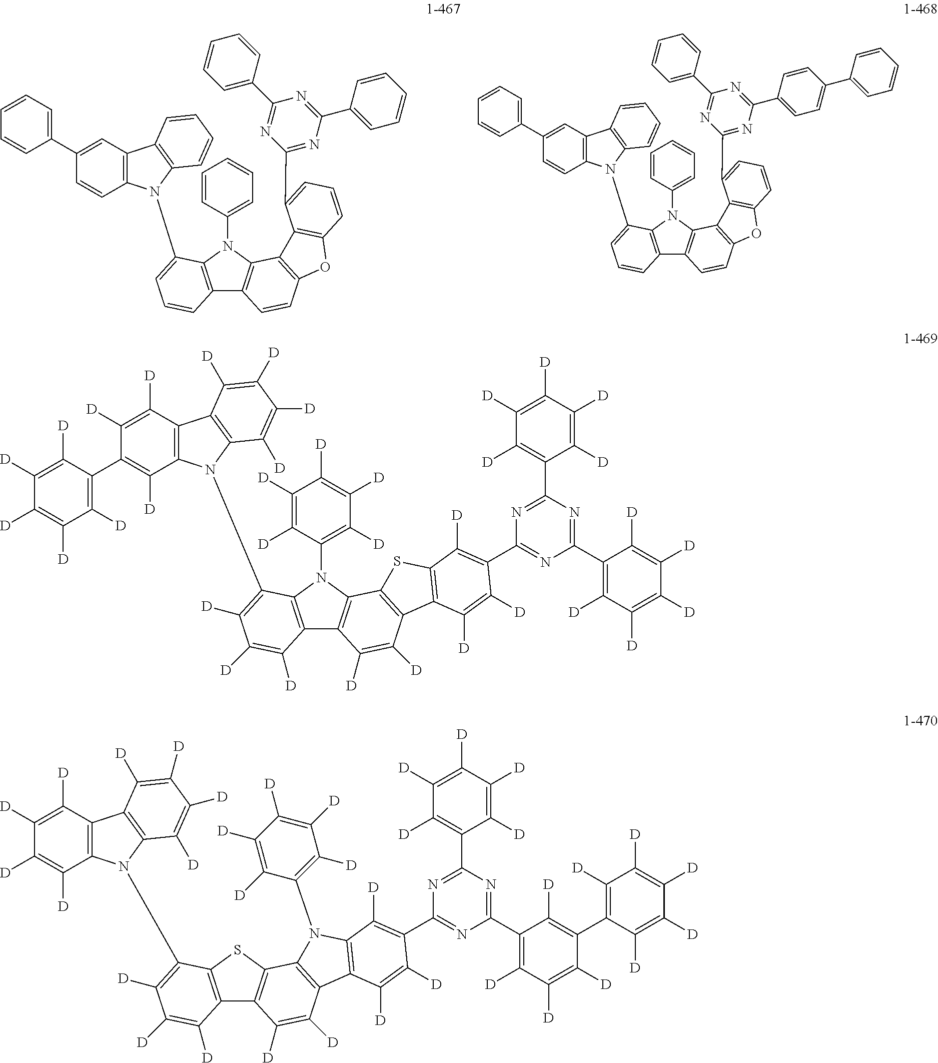

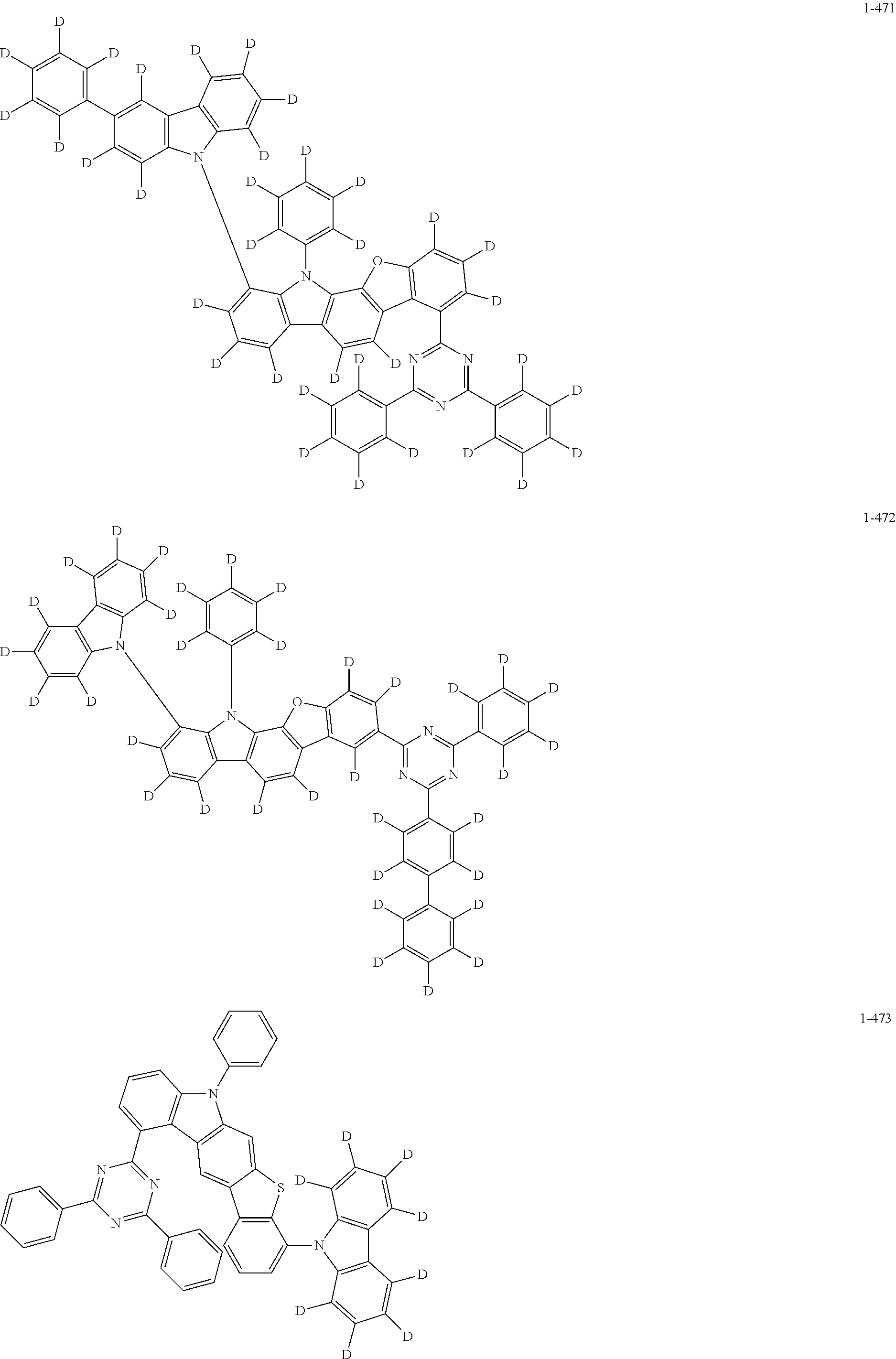

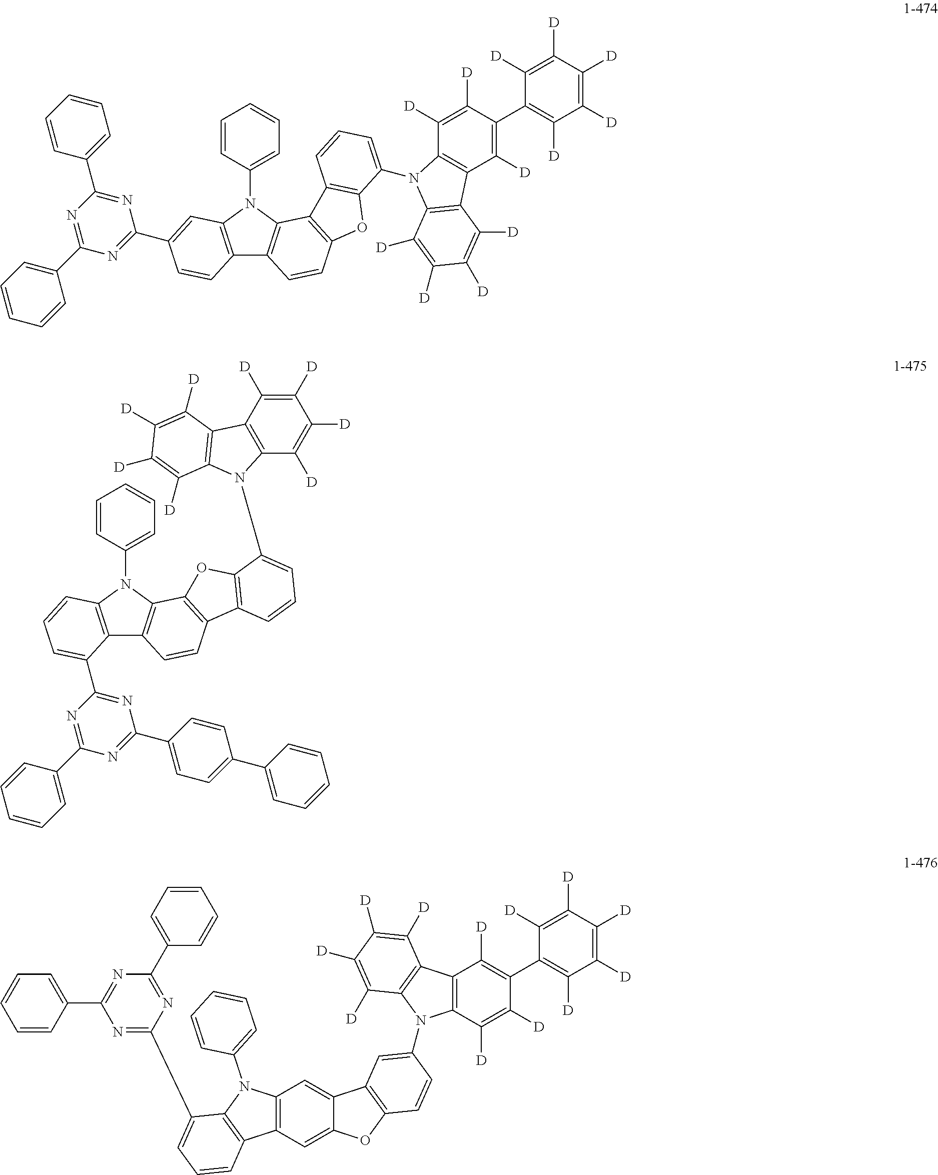

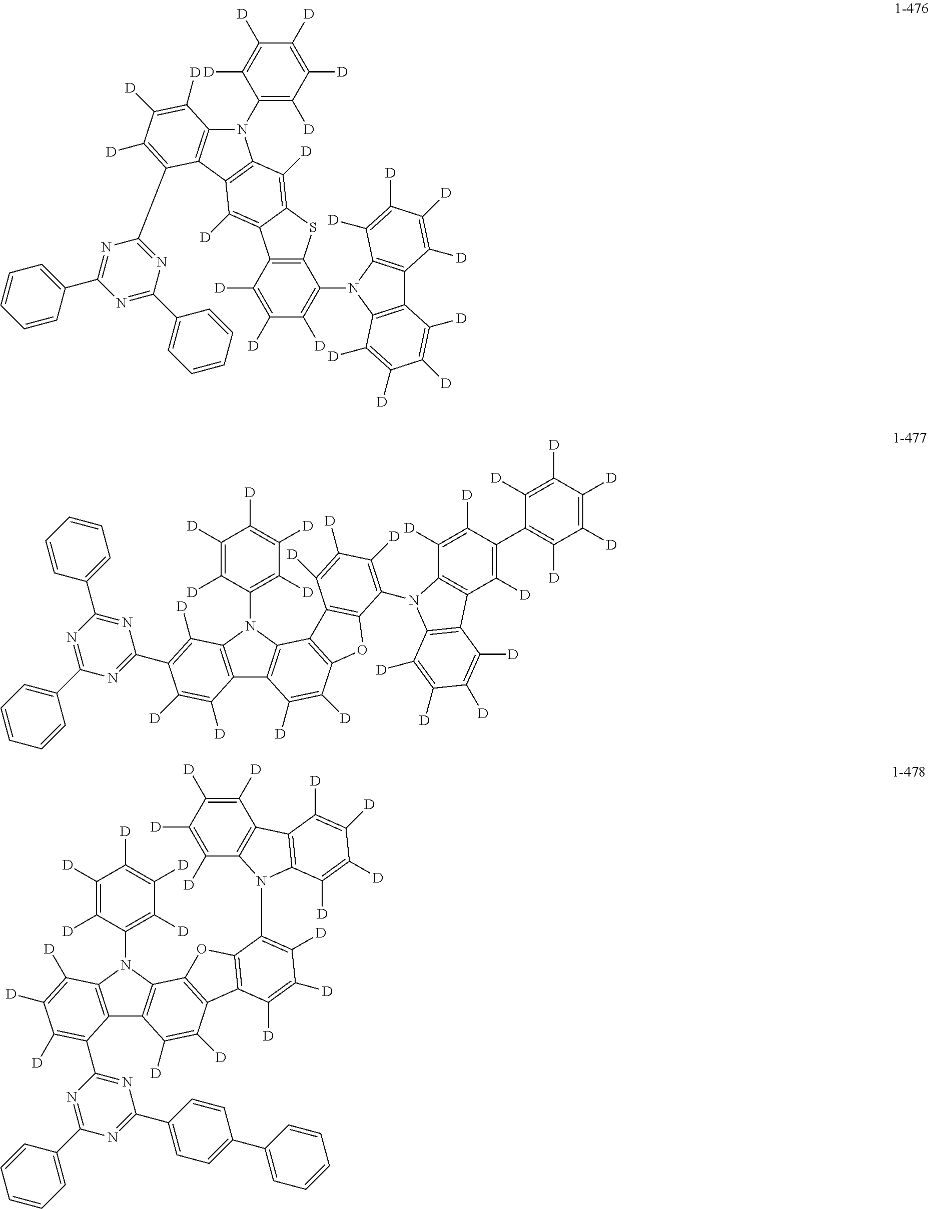

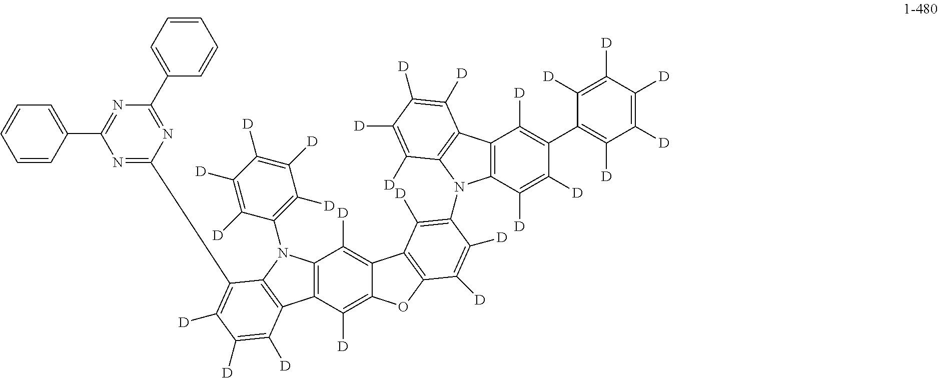 [SMILES: [2H]c1c([2H])c([2H])c(-c2c([2H])c([2H])c3c(c2[2H])c2c([2H])c([2H])c([2H])c([2H])c2n3-c2c([2H])c([2H])c3oc4c([2H])c5c6c([2H])c([2H])c([2H])c(-c7nc(-c8ccccc8)nc(-c8ccccc8)n7)c6n(-c6c([2H])c([2H])c([2H])c([2H])c6[2H])c5c([2H])c4c3c2[2H])c([2H])c1[2H]]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

Meanwhile, the compound has a high glass transition temperature (Tg) and thus has excellent thermal stability. The increase in thermal stability becomes an important factor for providing a device with driving stability.

Further, in an exemplary embodiment of the present application, provided is an organic light emitting device including: a first electrode; a second electrode provided to face the first electrode; and an organic material layer having one or more layers provided between the first electrode and the second electrode, in which one or more layers of the organic material layer include the heterocyclic compound represented by Chemical Formula 1.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocyclic compound according to Chemical Formula 1 may be used as a material for the blue organic light emitting device.

In an exemplary embodiment of the present application, the organic light emitting device may be a green organic light emitting device, and the heterocyclic compound represented by Chemical Formula 1 may be used as a material for the green organic light emitting device.

In an exemplary embodiment of the present application, the organic light emitting device may be a red organic light emitting device, and the heterocyclic compound represented by Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for a light emitting layer of the blue organic light emitting device.

In an exemplary embodiment of the present application, the organic light emitting device may be a green organic light emitting device, and the heterocyclic compound represented by Chemical Formula 1 may be used as a material for a light emitting layer of the green organic light emitting device.

In an exemplary embodiment of the present application, the organic light emitting device may be a red organic light emitting device, and the heterocyclic compound represented by Chemical Formula 1 may be used as a material for a light emitting layer of the red organic light emitting device.

The specific content on the heterocyclic compound represented by Chemical Formula 1 is the same as that described above.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In the organic light emitting device of the present invention, the organic material layer includes a light emitting layer, and the light emitting layer may include the heterocyclic compound of Chemical Formula 1.

In the organic light emitting device of the present invention, the organic material layer includes a light emitting layer, and the light emitting layer may include the heterocyclic compound of Chemical Formula 1 as a light emitting layer host.

In the organic light emitting device according to an exemplary embodiment of the present application, provided is an organic light emitting device in which the organic material layer including the heterocyclic compound represented by Chemical Formula 1 further includes a heterocyclic compound represented by the following Chemical Formula 2.

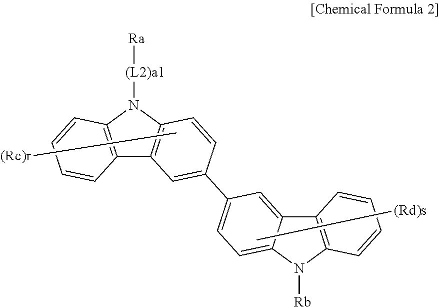

In Chemical Formula 2,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′, or two or more adjacent groups are bonded to each other to form a substituted or unsubstituted aromatic hydrocarbon ring or a substituted or unsubstituted hetero ring,
L2 is a direct bond; a substituted or unsubstituted C6 to C60 arylene group; or a substituted or unsubstituted C2 to C60 heteroarylene group,
Ra and Rb are the same as or different from each other, and are each independently —CN; —SiRR′R″; —P(═O)RR′; a substituted or unsubstituted C6 to C60 aryl group; or a substituted or unsubstituted C2 to C60 heteroaryl group,
R, R′ and R″ are the same as or different from each other, and are each independently hydrogen; deuterium; a substituted or unsubstituted C1 to C60 alkyl group; or a substituted or unsubstituted C6 to C60 aryl group,
a1 is an integer from 0 to 4,
r and s are an integer from 0 to 7, and
when a1, s and r are 2 or higher, substituents in the parenthesis are the same as or different from each other.

In an exemplary embodiment of the present application, L2 may be a direct bond; a substituted or unsubstituted C6 to C60 arylene group; or a substituted or unsubstituted C2 to C60 heteroarylene group.

In another exemplary embodiment, L2 may be a direct bond; a substituted or unsubstituted C6 to C40 arylene group; or a substituted or unsubstituted C2 to C40 heteroarylene group.

In still another exemplary embodiment, L2 may be a direct bond; a C6 to C40 arylene group; or a C2 to C40 heteroarylene group.

In yet another exemplary embodiment, L2 may be a direct bond; a substituted or unsubstituted phenylene group; a substituted or unsubstituted biphenylene group; or a substituted or unsubstituted divalent dibenzofuran group.

In still yet another exemplary embodiment, L2 may be a direct bond; a phenylene group; a biphenylene group; a divalent dibenzothiophene group; a divalent dimethylfluorene group; or a divalent dibenzofuran group.

In an exemplary embodiment of the present application, L2 may be substituted with deuterium.

In an exemplary embodiment of the present application, Ra and Rb are the same as or different from each other, and may be each independently —CN; —SiRR′R″; —P(═O)RR′; a substituted or unsubstituted C6 to C60 aryl group; or a substituted or unsubstituted C2 to C60 heteroaryl group.

In another exemplary embodiment, Ra may be —CN; —SiRR′R″; —P(═O)RR′; a substituted or unsubstituted C6 to C60 aryl group; or a substituted or unsubstituted C2 to C60 heteroaryl group.

In still another exemplary embodiment, Ra may be —CN; —SiRR′R″; —P(═O)RR′; a C6 to C40 aryl group which is unsubstituted or substituted with a C1 to C40 alkyl group or a C6 to C40 aryl group; or a C2 to C60 heteroaryl group which is unsubstituted or substituted with a C6 to C40 aryl group or a C2 to C40 heteroaryl group.

In yet another exemplary embodiment, Ra may be —CN; —SiRR′R″; —P(═O)RR′; a phenyl group; a biphenyl group; a terphenyl group; a dimethylfluorenyl group; a diphenylfluorenyl group; a spirobifluorenyl group; a dibenzothiophene group which is unsubstituted or substituted with a phenyl group or a dibenzofuran group; or a dibenzofuran group which is unsubstituted or substituted with a phenyl group or a dibenzofuran group.

In an exemplary embodiment of the present application, Rb may be a substituted or unsubstituted C6 to C60 aryl group; or a substituted or unsubstituted C2 to C60 heteroaryl group.

In another exemplary embodiment, Rb may be a C6 to C60 aryl group which is unsubstituted or substituted with a C1 to C40 alkyl group, —CN, SiRR′R″ or a C6 to C40 aryl group.

In still another exemplary embodiment, Rb may be a C6 to C40 aryl group which is unsubstituted or substituted with a C1 to C40 alkyl group, —CN, SiRR′R″ or a C6 to C40 aryl group.

In yet another exemplary embodiment, Rb may be a phenyl group which is unsubstituted or substituted with —CN or SiRR′R″; a biphenyl group which is unsubstituted or substituted with a phenyl group; a terphenyl group; a dimethylfluorenyl group.

In an exemplary embodiment of the present application, Ra and Rb may be substituted with deuterium.

In an exemplary embodiment of the present application, -(L2)a-Ra and Rb of Chemical Formula 2 may be different from each other.

In an exemplary embodiment of the present application, -(L2)a-Ra and Rb of Chemical Formula 2 may be the same as each other.

In yet another exemplary embodiment, R, R′, and R″ may be a substituted or unsubstituted phenyl group.

In yet another exemplary embodiment, R, R′, and R″ may be a phenyl group.

In an exemplary embodiment of the present application, the deuterium content of Chemical Formula 2 may be 0% or more and 100% or less.

In another exemplary embodiment, the deuterium content of Chemical Formula 2 may be 10% or more and 100% or less.

In still another exemplary embodiment, the deuterium content of Chemical Formula 2 may be 0%, 100% or 10% to 80%.

In an exemplary embodiment of the present application, Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′, or two or more adjacent groups may be bonded to each other to form a substituted or unsubstituted aromatic hydrocarbon ring or a substituted or unsubstituted hetero ring.

In another exemplary embodiment, Rc and Rd are the same as or different from each other, and may be each independently selected from the group consisting of hydrogen; deuterium; a substituted or unsubstituted C1 to C60 alkyl group; a substituted or unsubstituted C6 to C60 aryl group; a substituted or unsubstituted C2 to C60 heteroaryl group; —SiRR′R″; —P(═O)RR′; and —NRR′.

In still another exemplary embodiment, Rc and Rd are the same as or different from each other, and may be each independently selected from the group consisting of hydrogen; deuterium; a substituted or unsubstituted C1 to C40 alkyl group; a substituted or unsubstituted C6 to C40 aryl group; a substituted or unsubstituted C2 to C40 heteroaryl group; —SiRR′R″; —P(═O)RR′; and —NRR′.

In yet another exemplary embodiment, Rc and Rd are the same as or different from each other, and may be each independently selected from the group consisting of hydrogen; deuterium; a C1 to C40 alkyl group; a C6 to C40 aryl group; a C2 to C40 heteroaryl group; —SiRR′R″; —P(═O)RR′; and —NRR′.

In still yet another exemplary embodiment, Rc and Rd are the same as or different from each other, and may be each independently selected from the group consisting of hydrogen; deuterium; a C1 to C20 alkyl group; a C6 to C20 aryl group; a C2 to C20 heteroaryl group; —SiRR′R″; —P(═O)RR′; and —NRR′.

In a further exemplary embodiment, Rc and Rd are the same as or different from each other, and may be each independently hydrogen; or deuterium.

In an exemplary embodiment of the present application, r is 7, and Rc may be hydrogen.

In an exemplary embodiment of the present application, r is 7, and Rc may be deuterium.

In an exemplary embodiment of the present application, r is 7, and Rc may be hydrogen; or deuterium.

In an exemplary embodiment of the present application, s is 7, and Rd may be hydrogen.

In an exemplary embodiment of the present application, s is 7, and Rd may be deuterium.

In an exemplary embodiment of the present application, s is 7, and Rd may be hydrogen; or deuterium.

When both the compound of Chemical Formula 1 and the compound of Chemical Formula 2 are included in the organic material layer of the organic light emitting device, better efficiency and service life effects are exhibited. From this result, it can be expected that an exciplex phenomenon will occur when both compounds are included.

The exciplex phenomenon is a phenomenon in which energy with a magnitude of the HOMO level of a donor (p-host) and the LUMO level of an acceptor (n-host) is released due to an electron exchange between two molecules. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life.

In an exemplary embodiment of the present application, the heterocyclic compound of Chemical Formula 2 may be represented by any one of the following compounds.

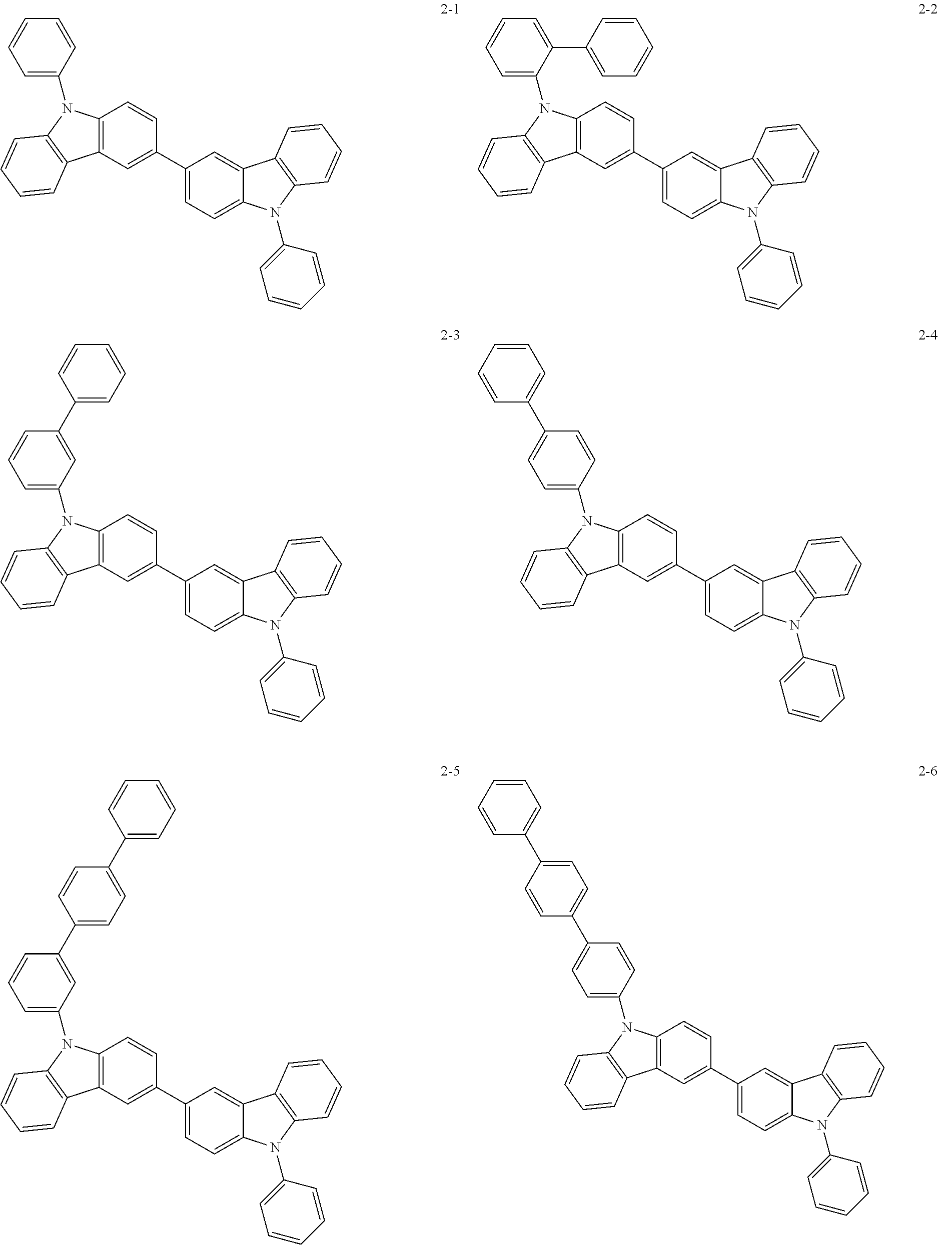 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

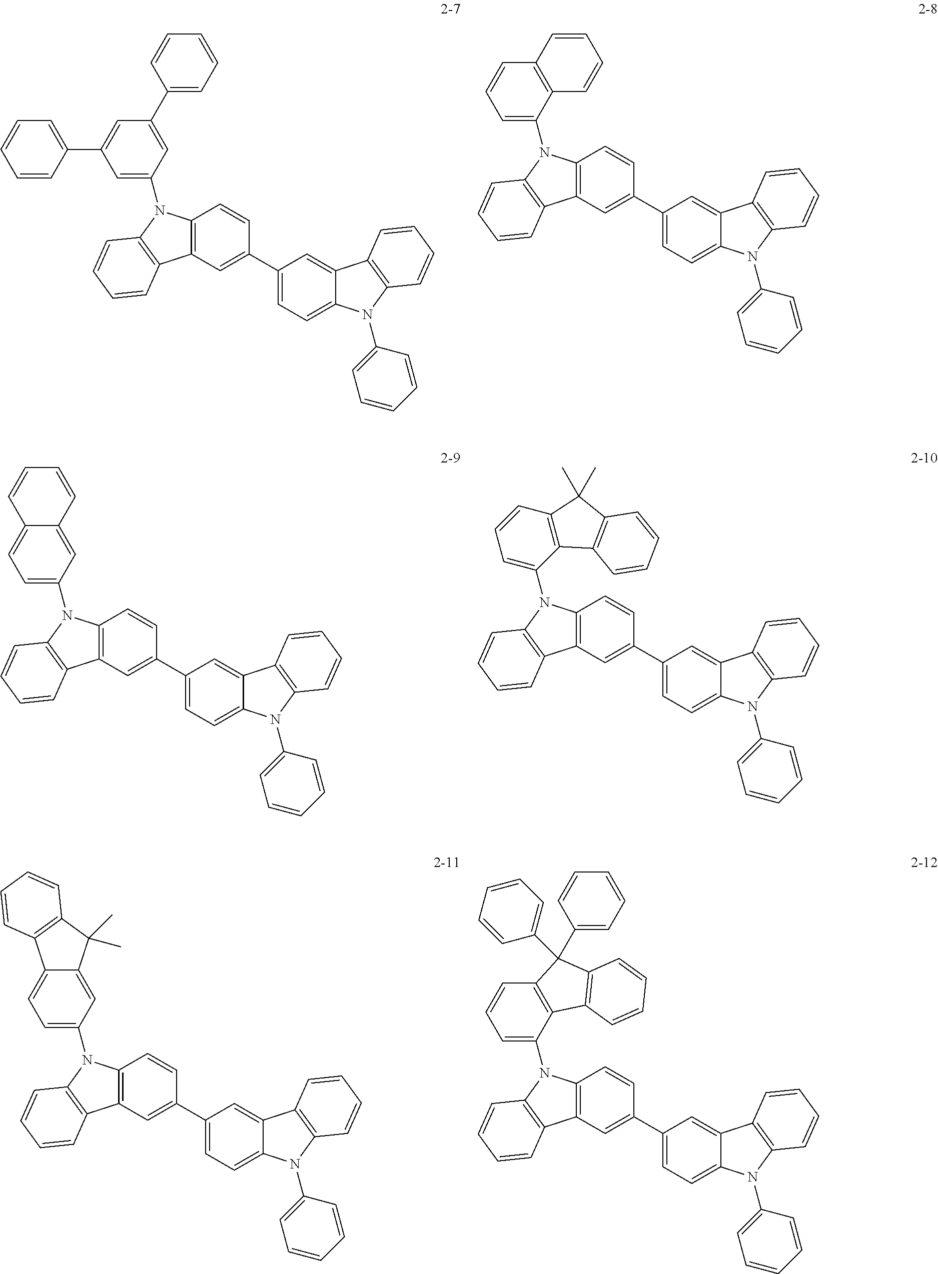 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

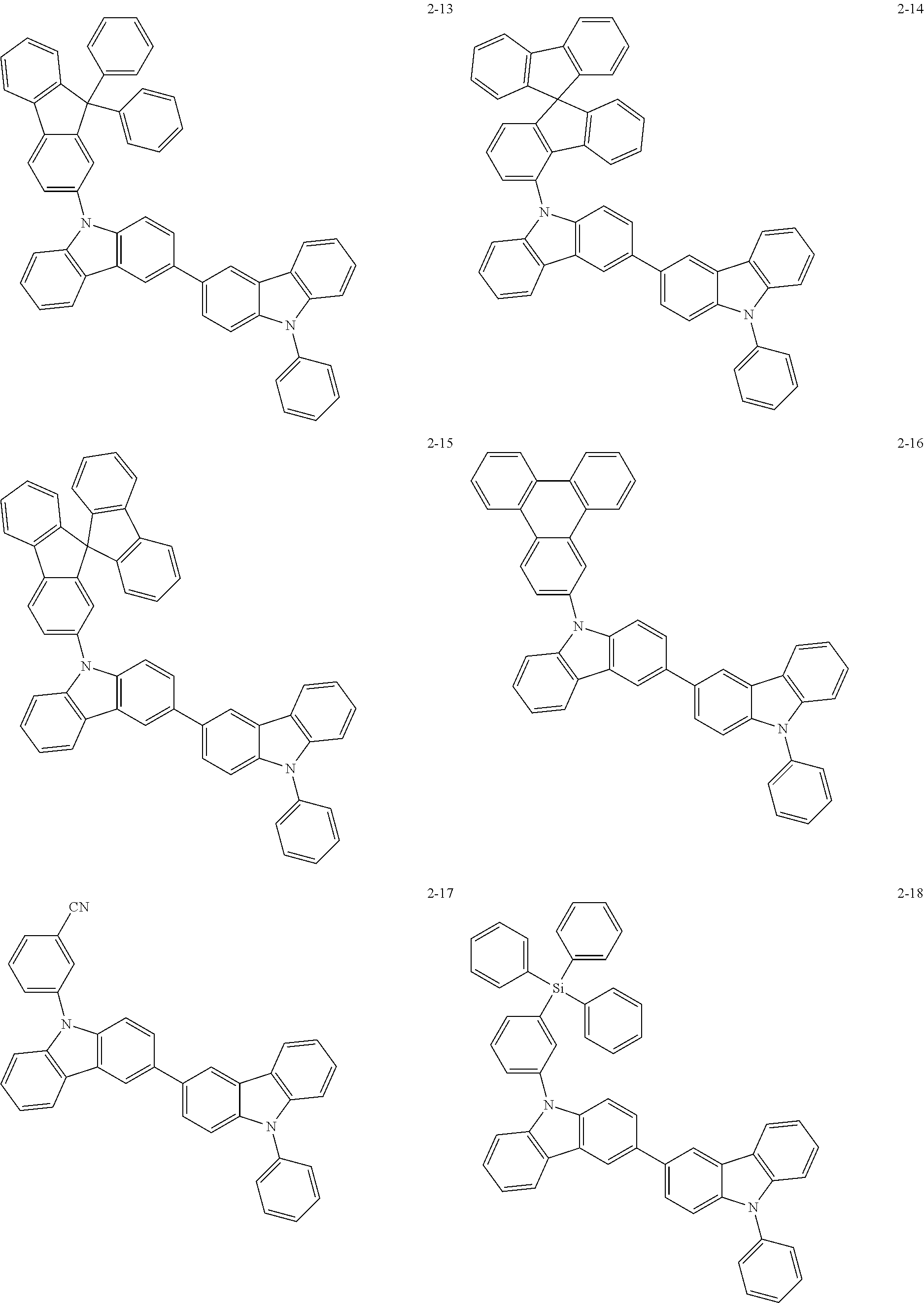

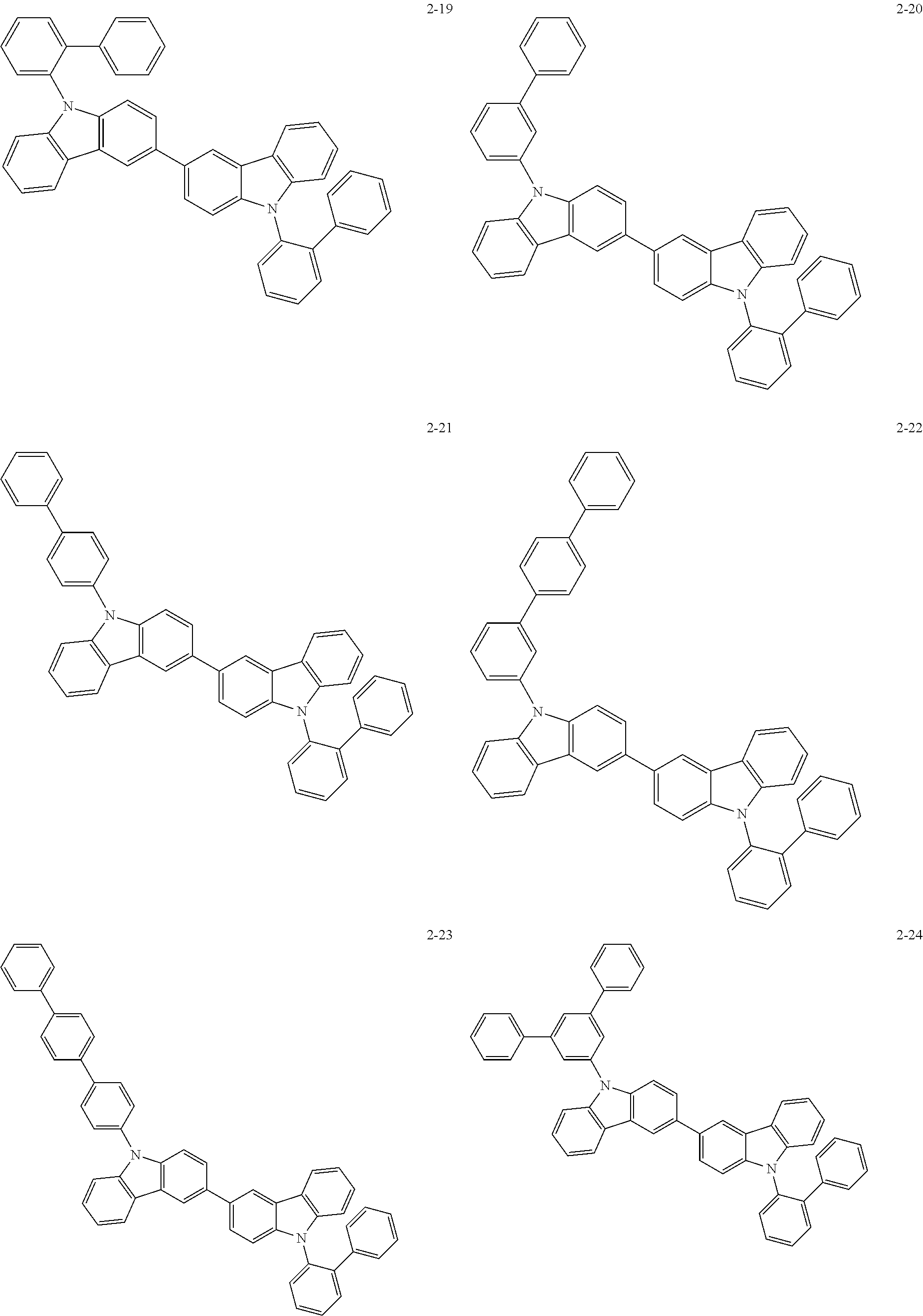

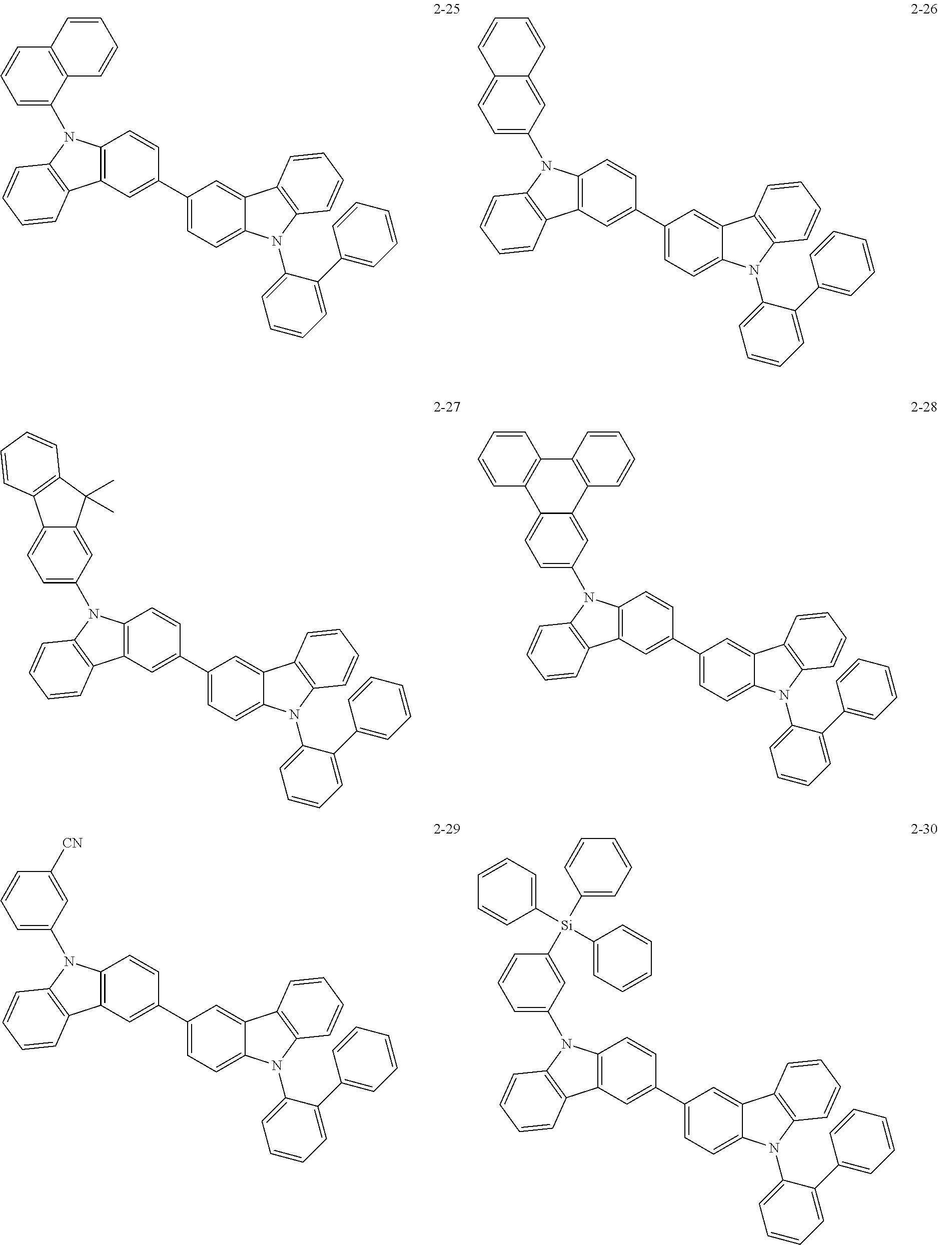

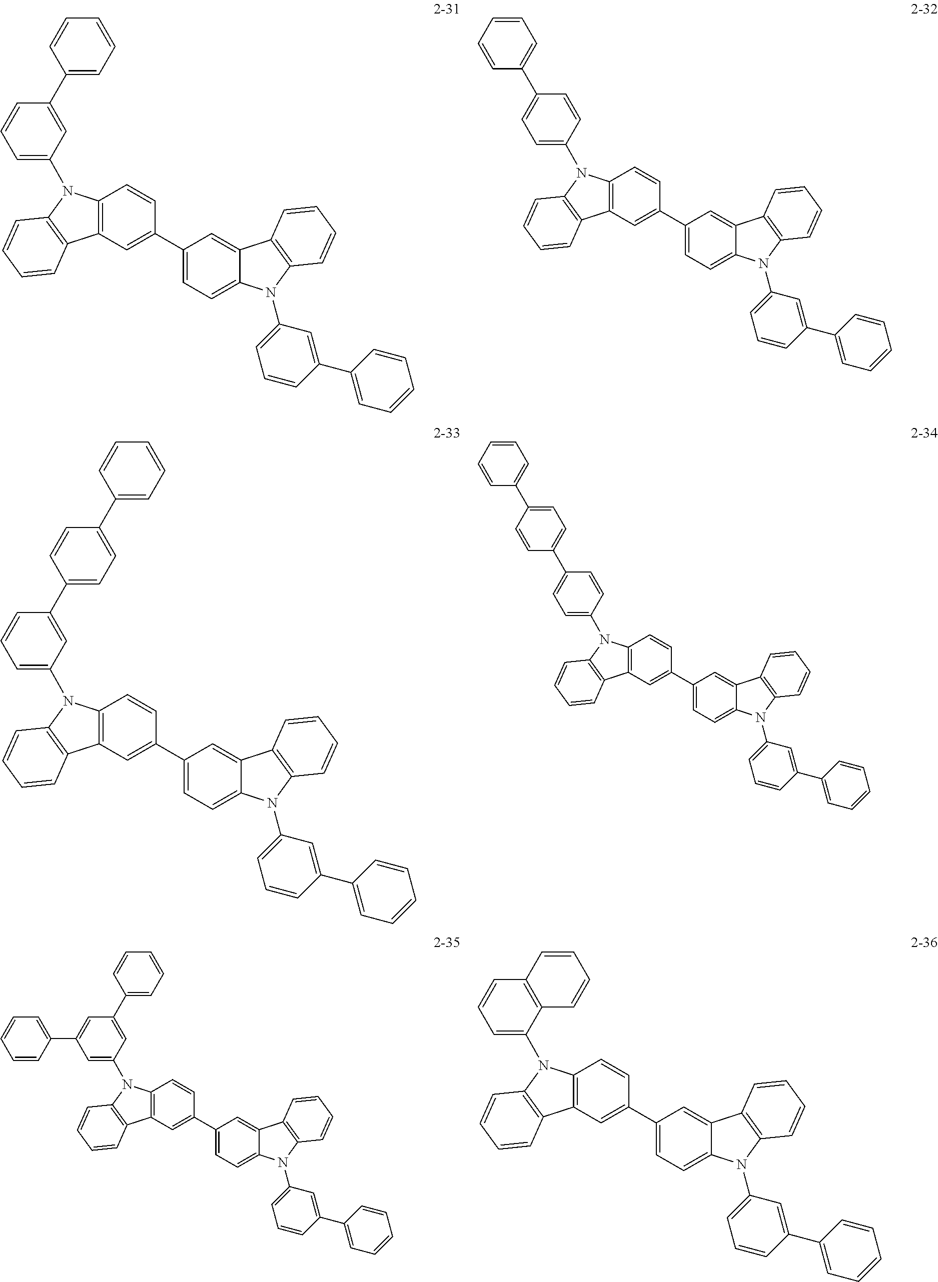

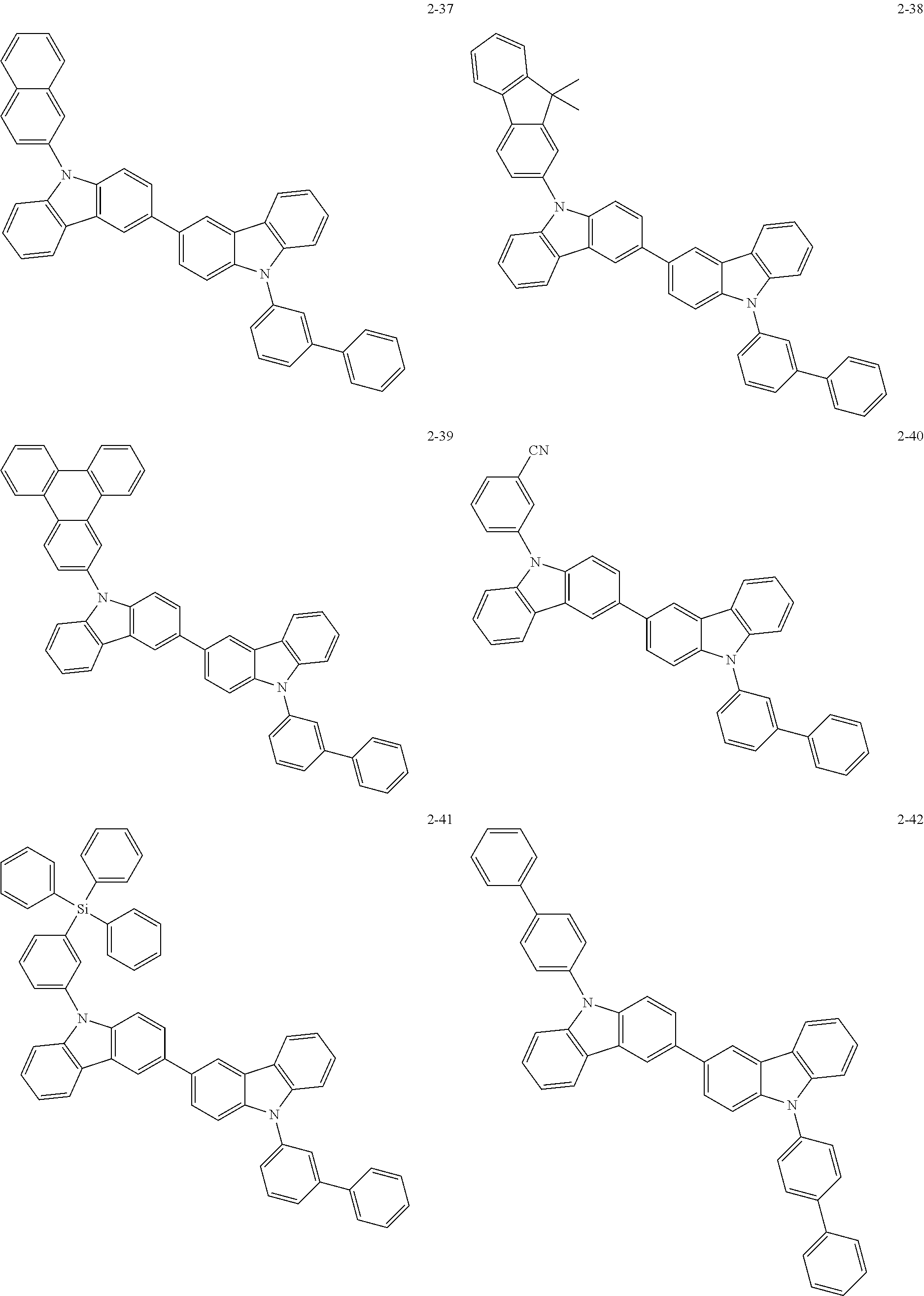

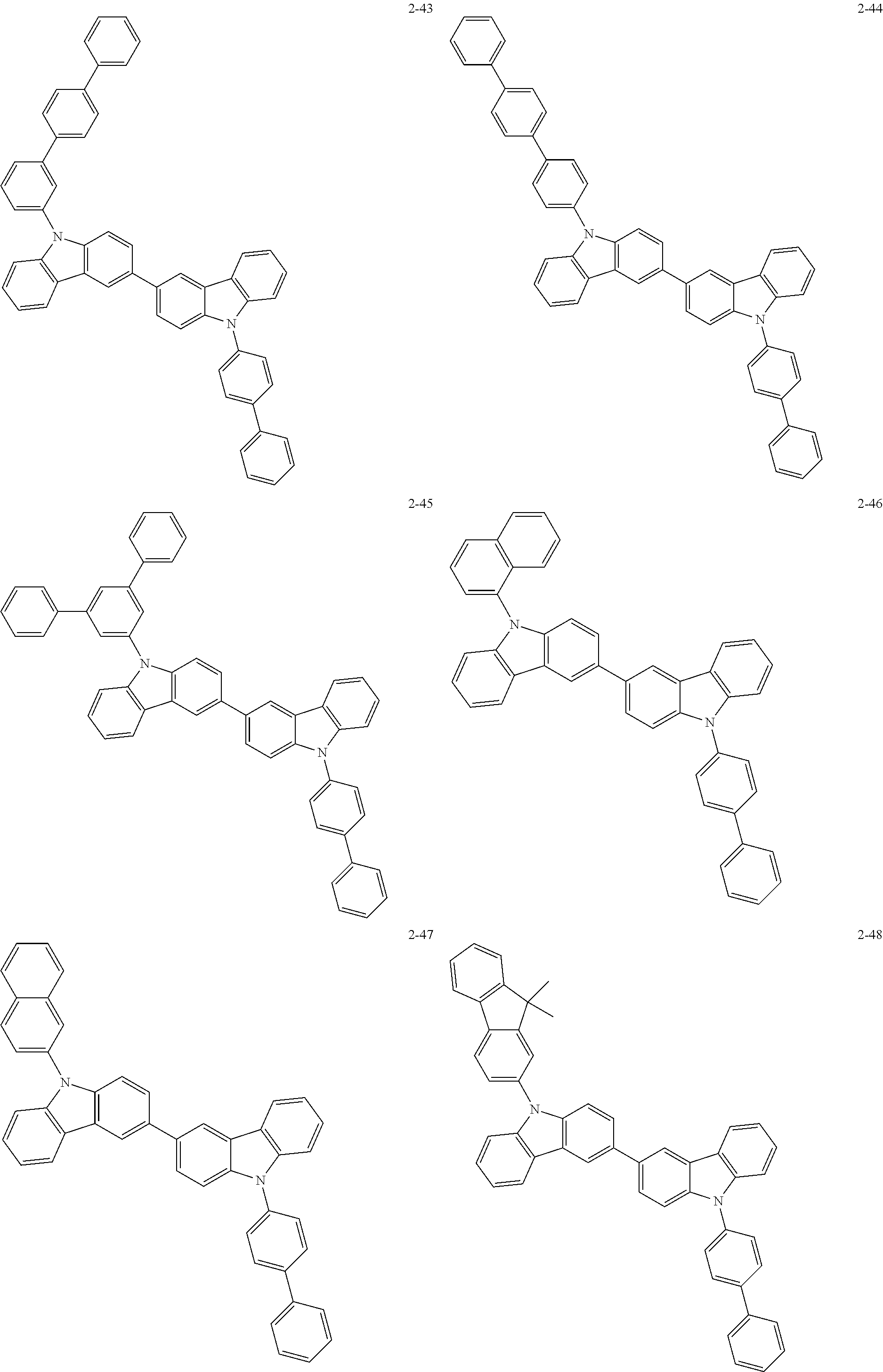

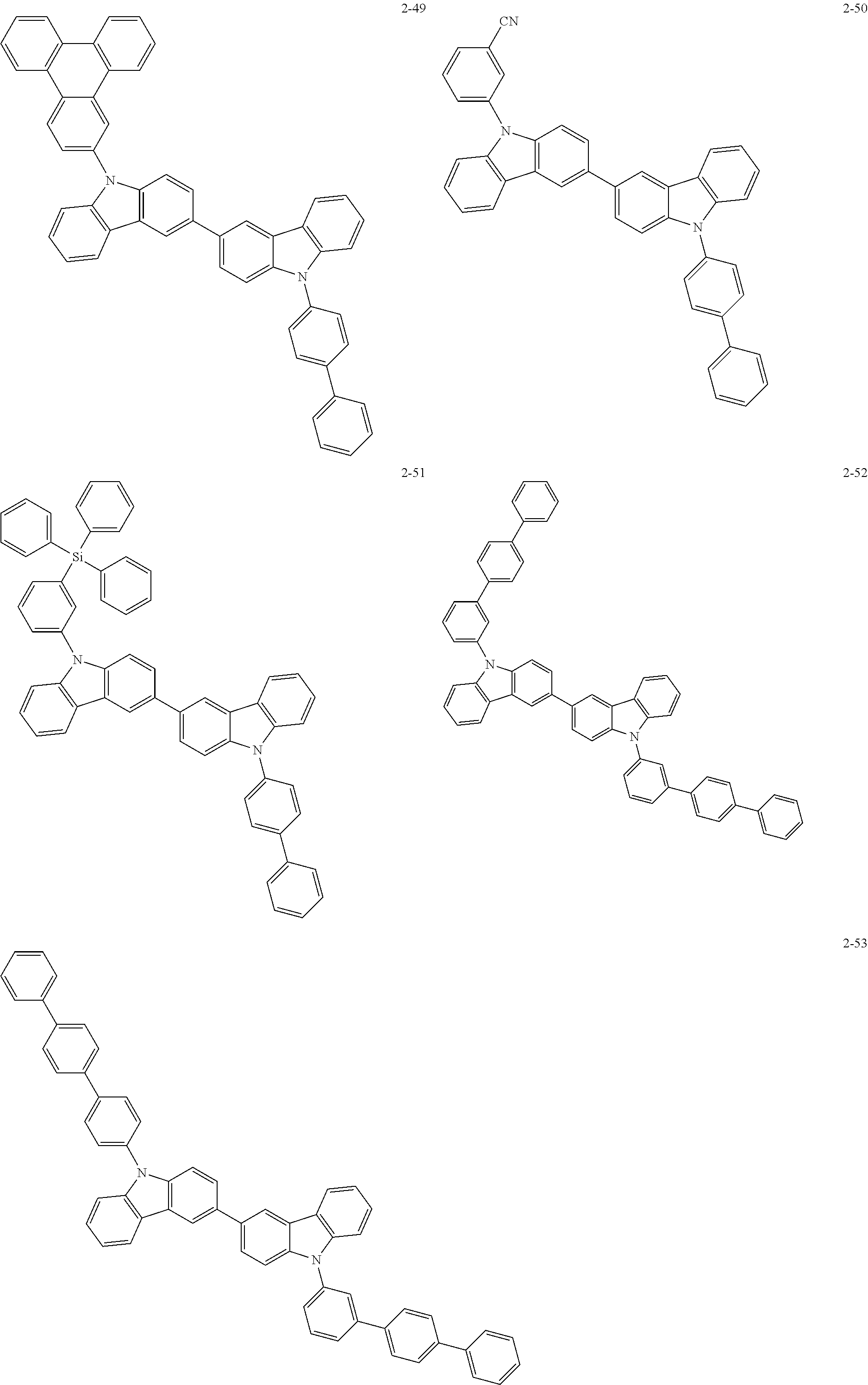

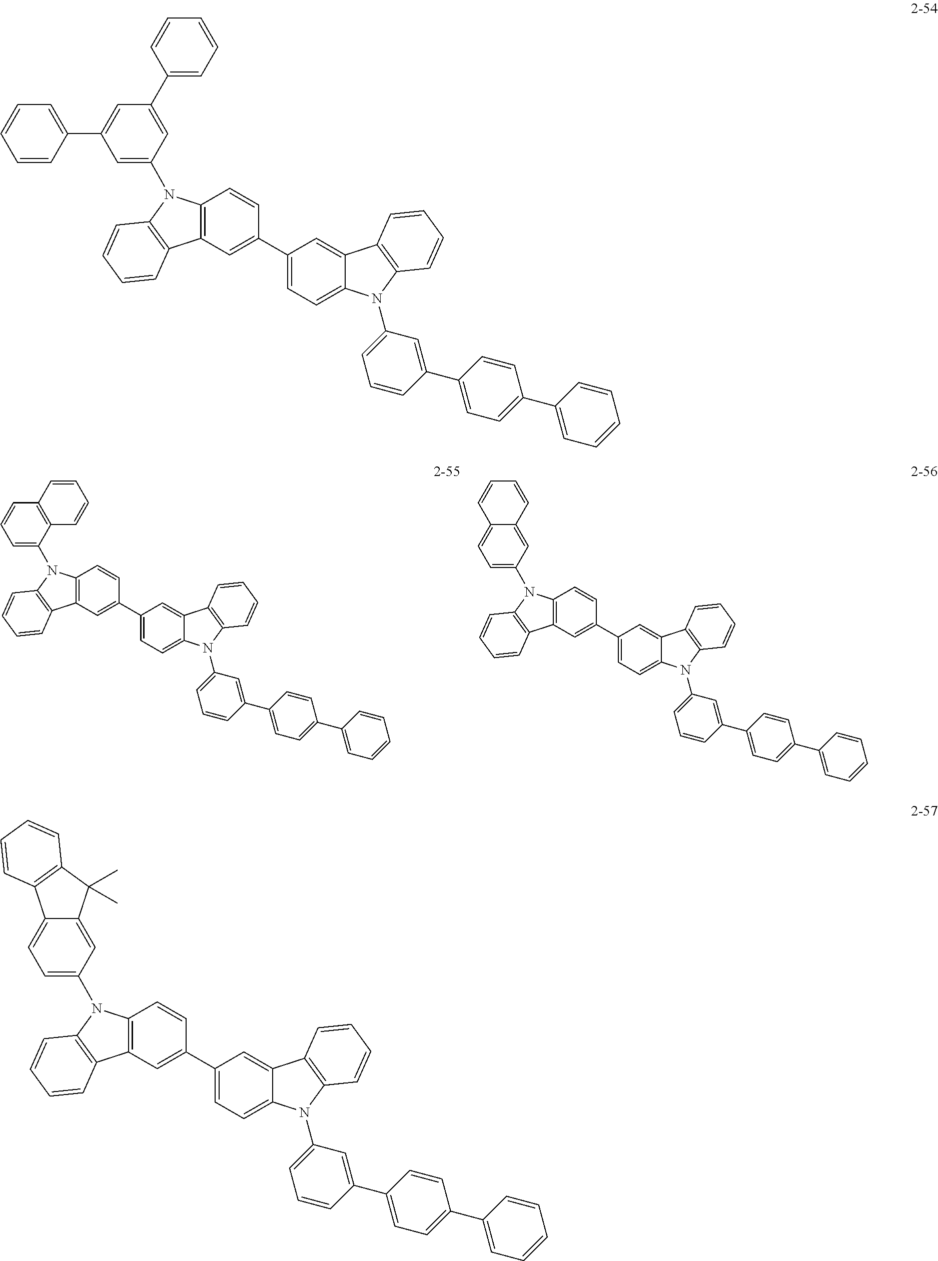 [SMILES: c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1 ; CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21]

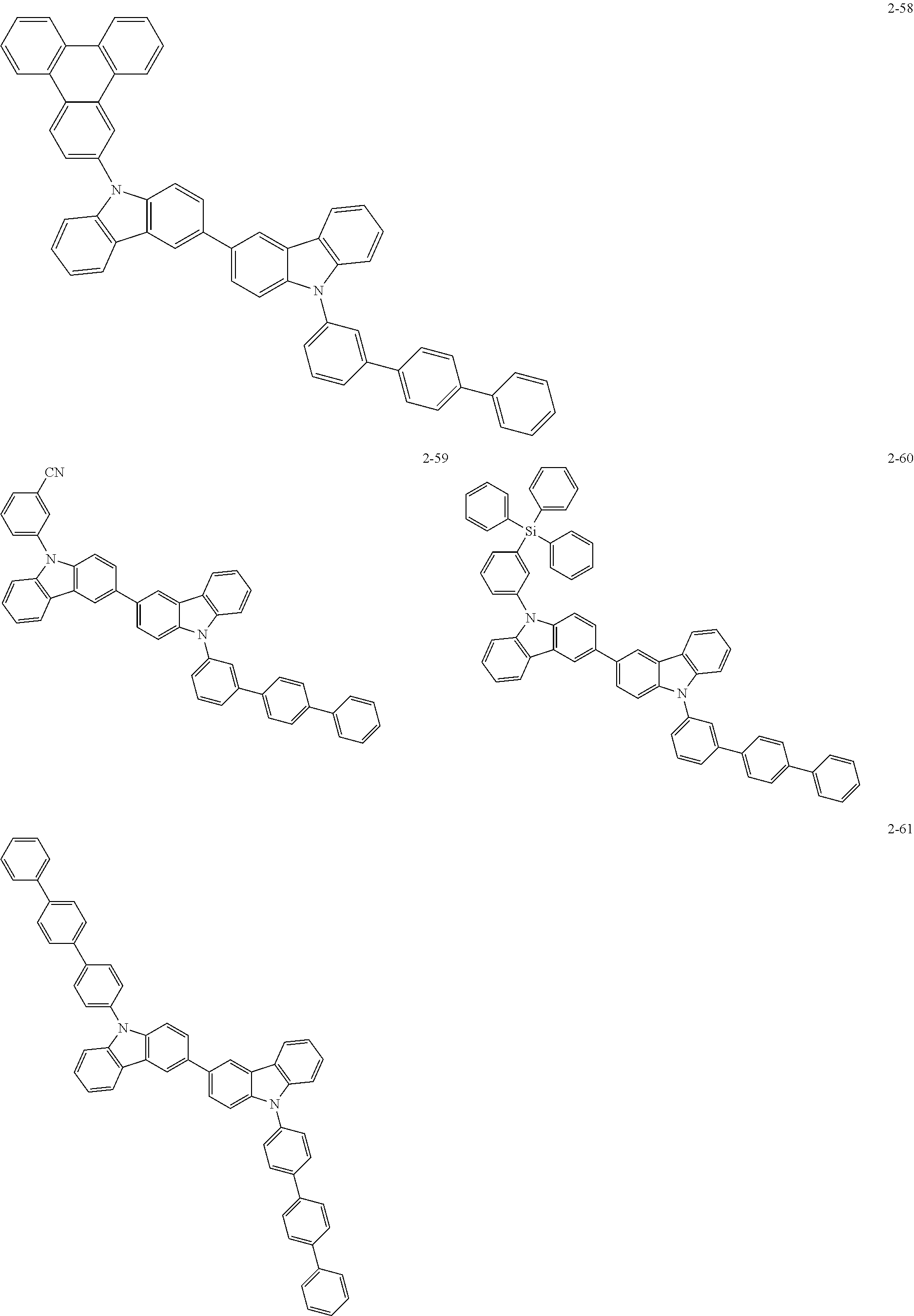

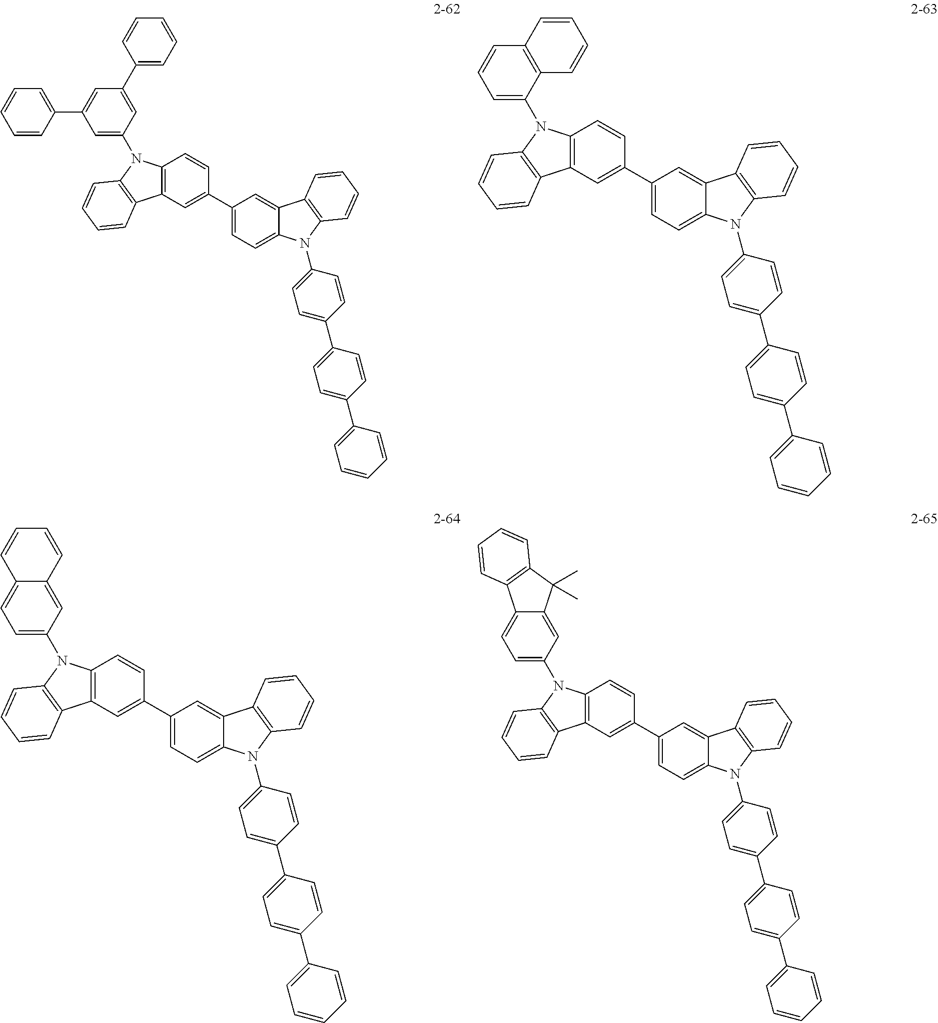 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21]

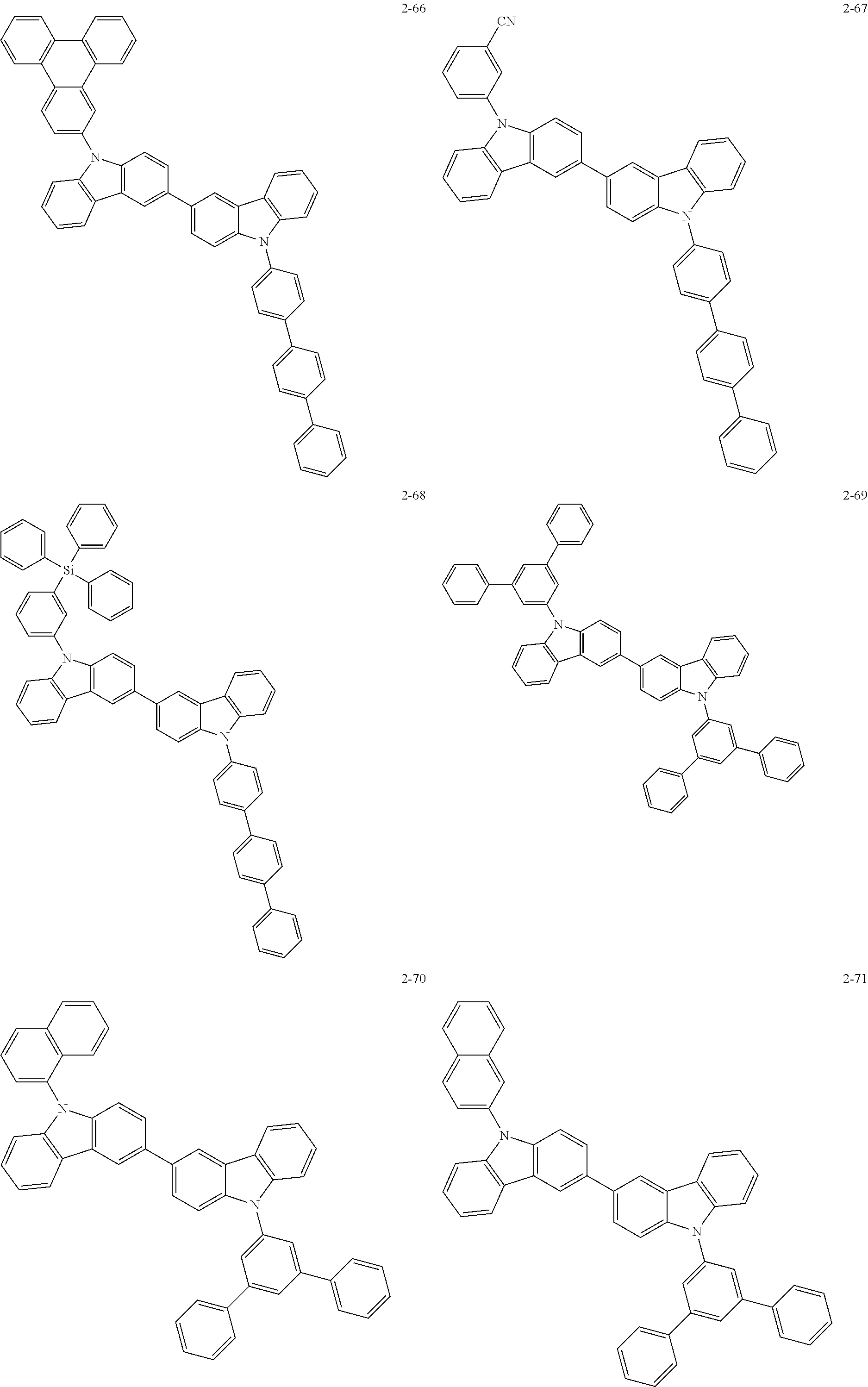

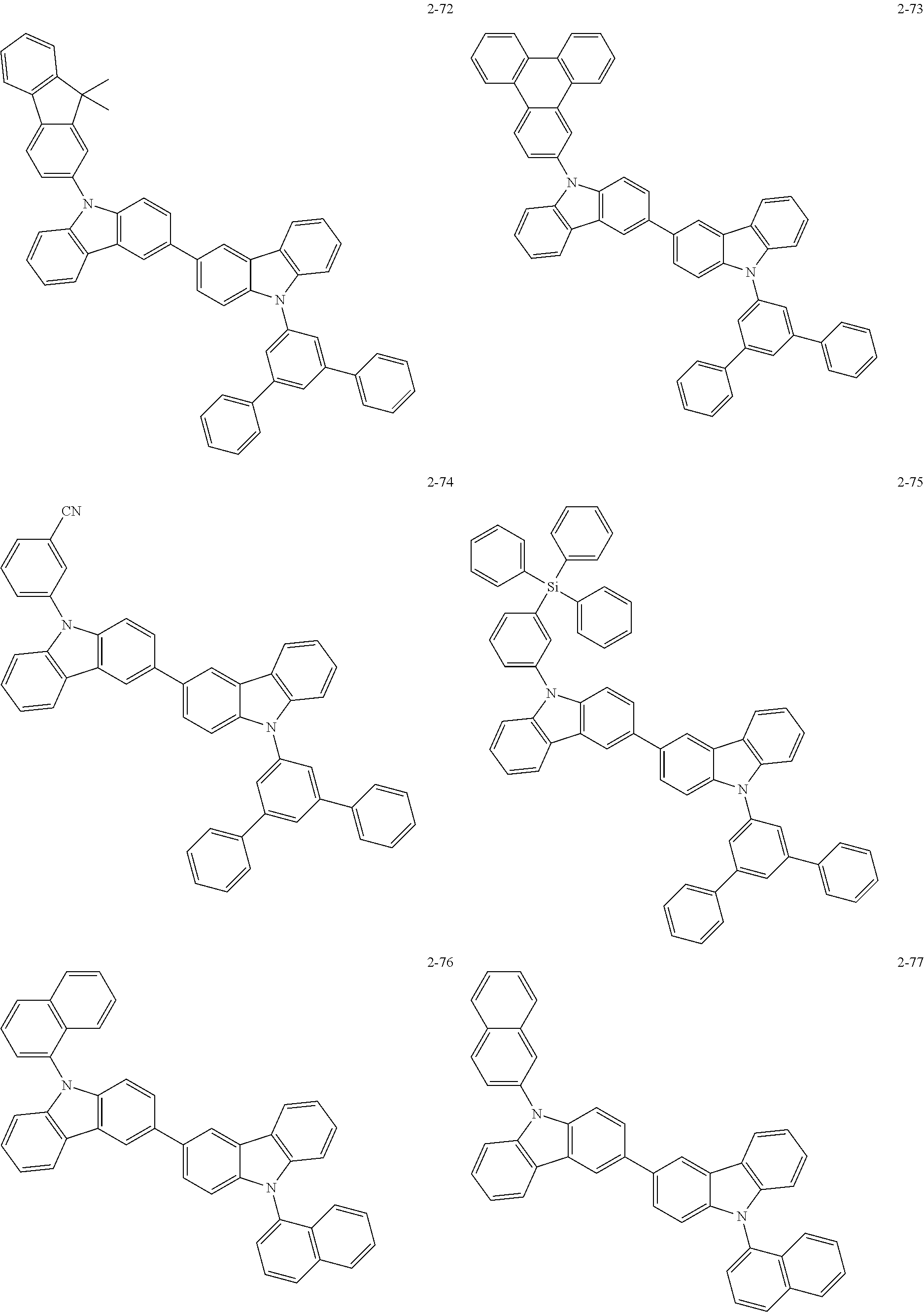

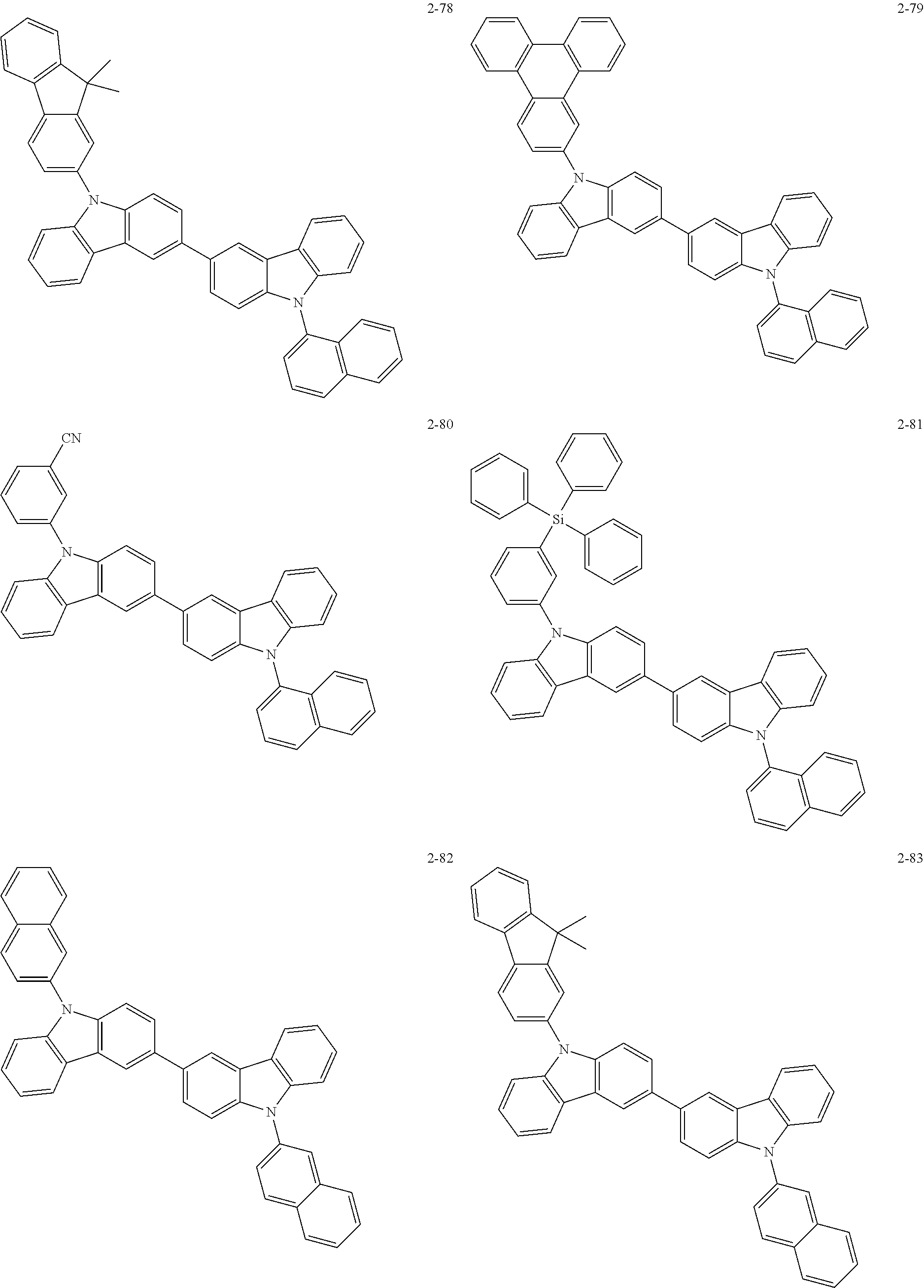

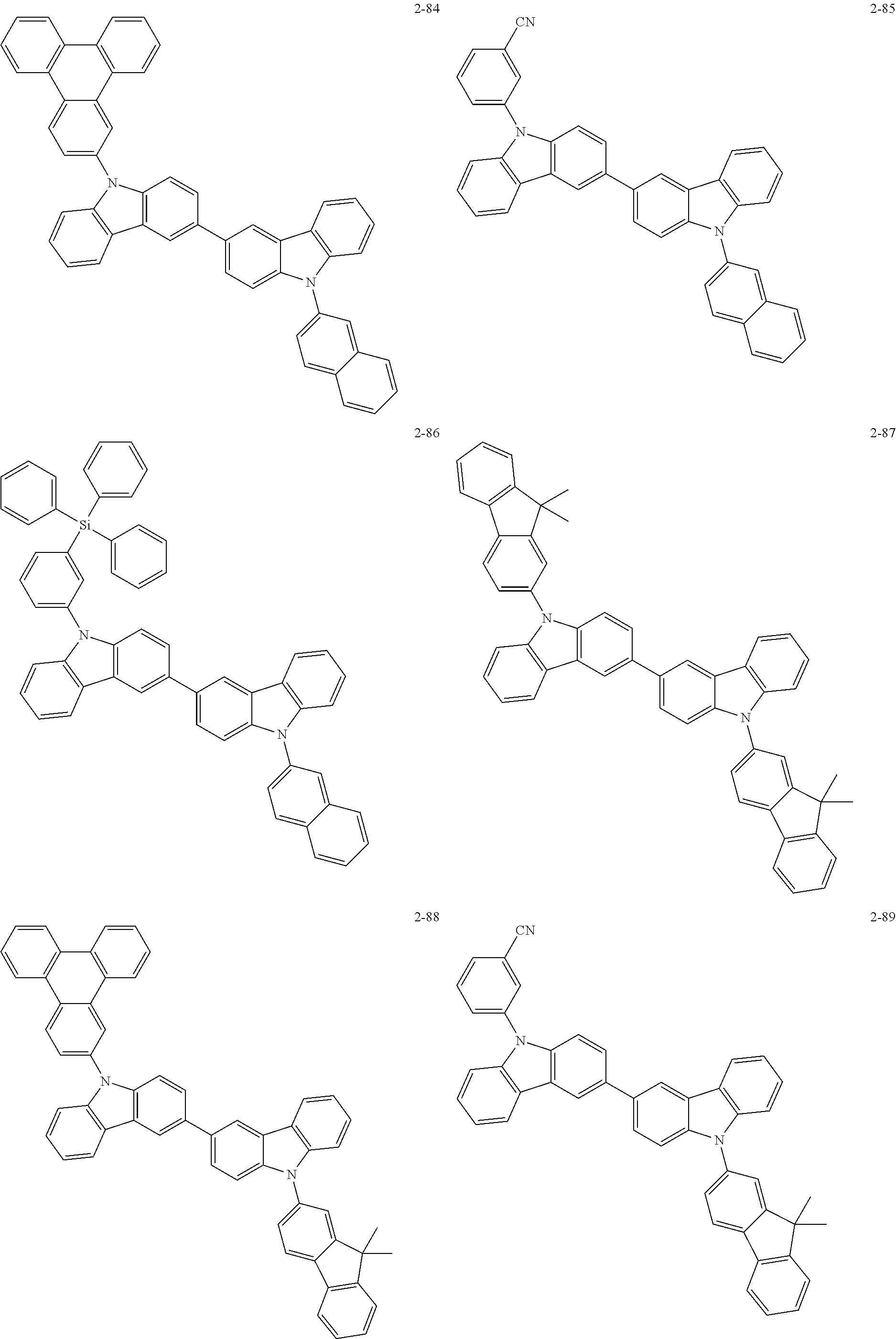

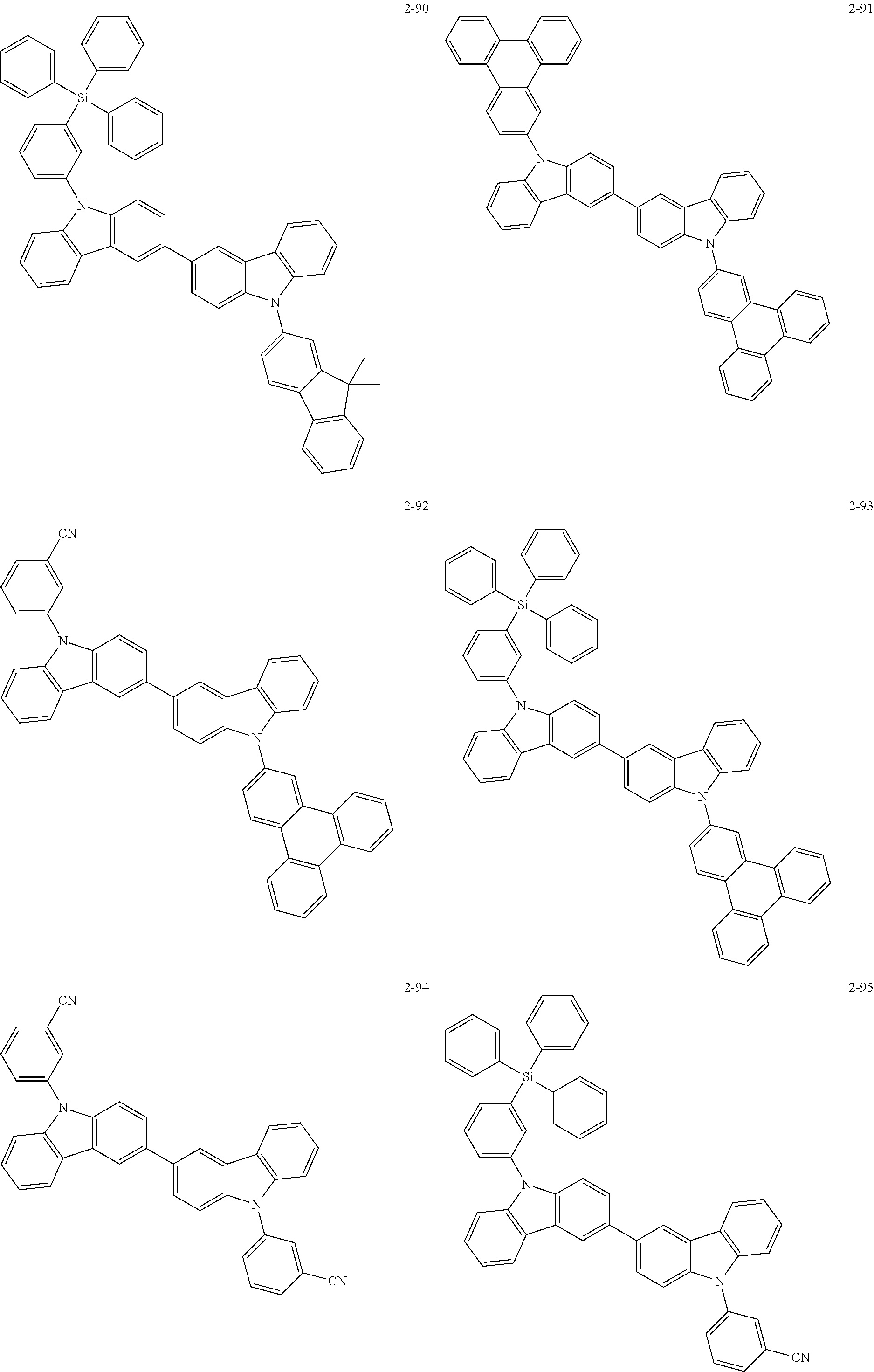

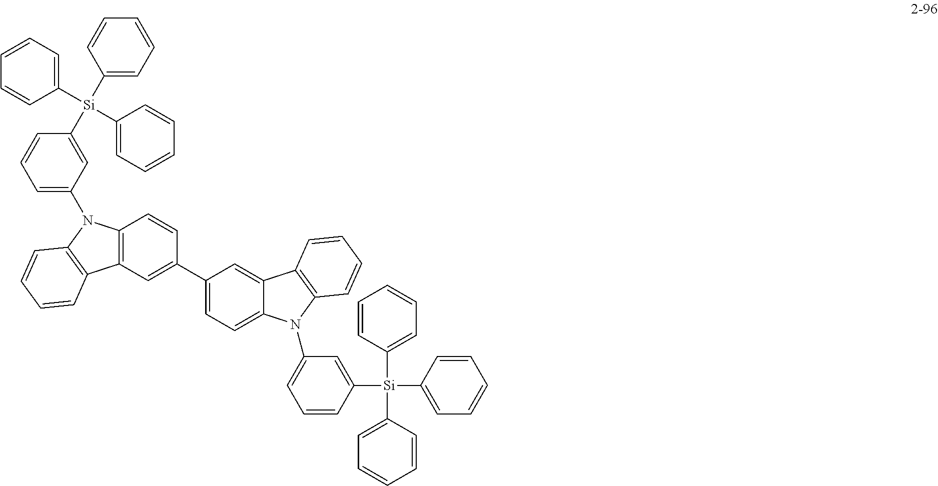 [SMILES: c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

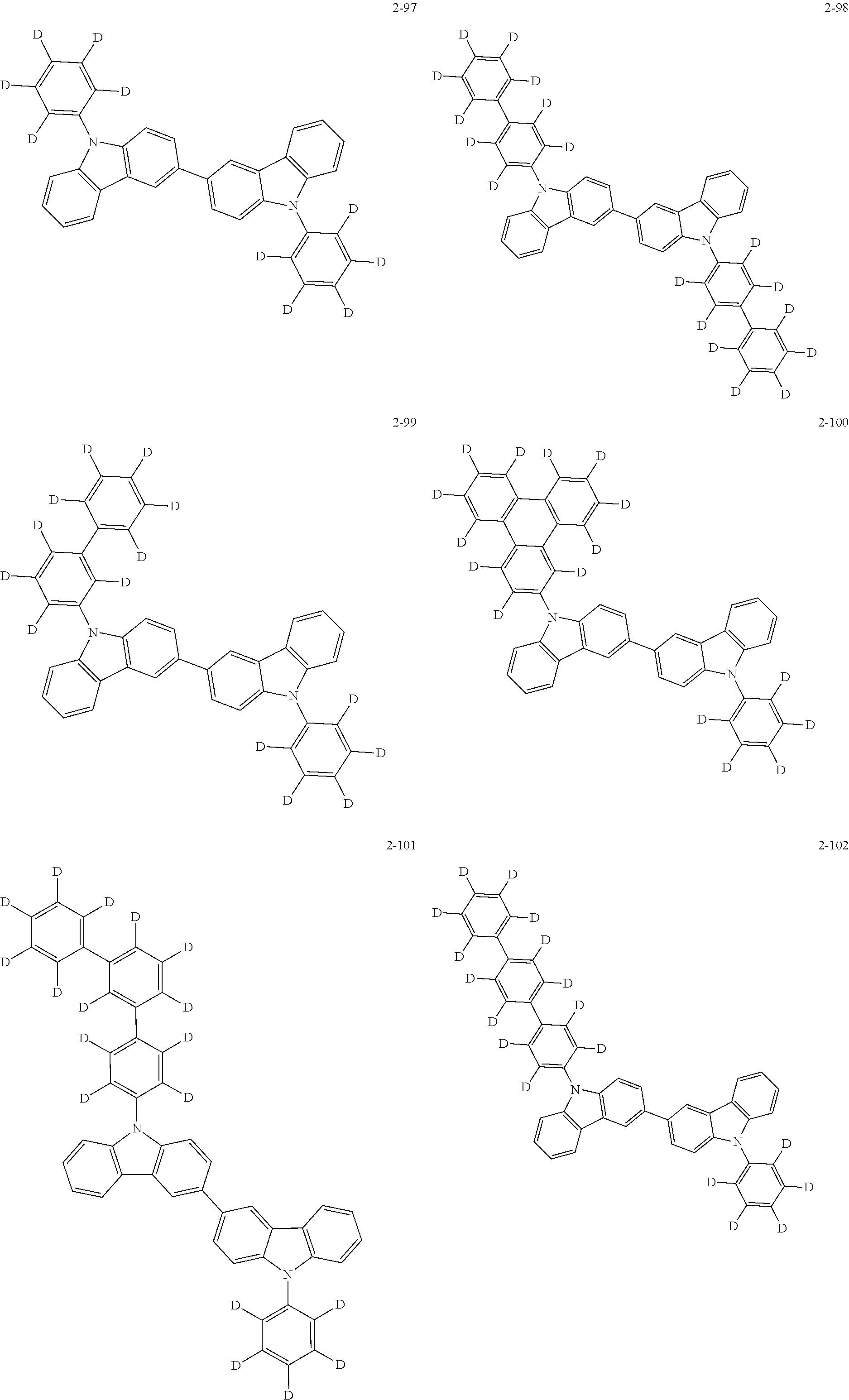

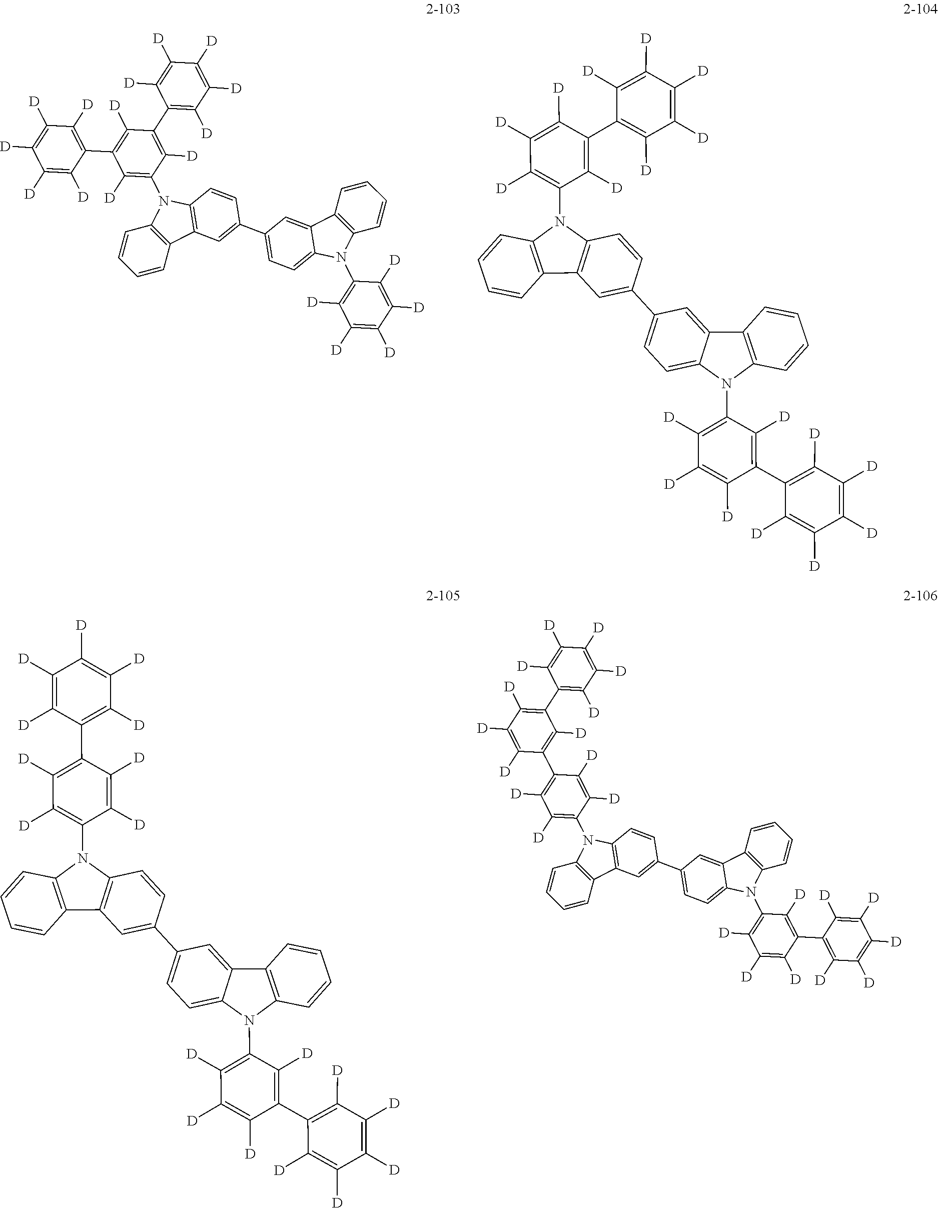

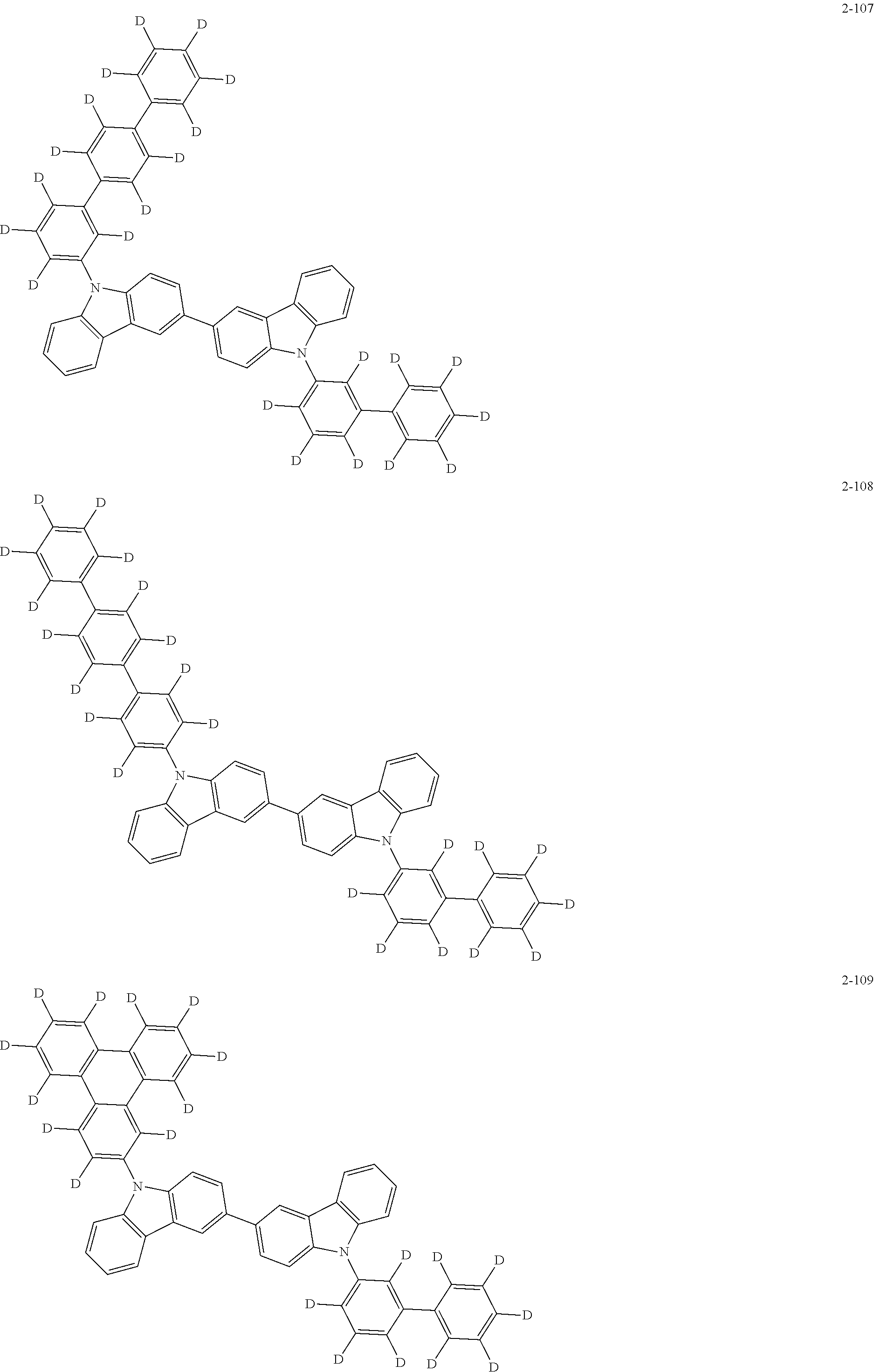

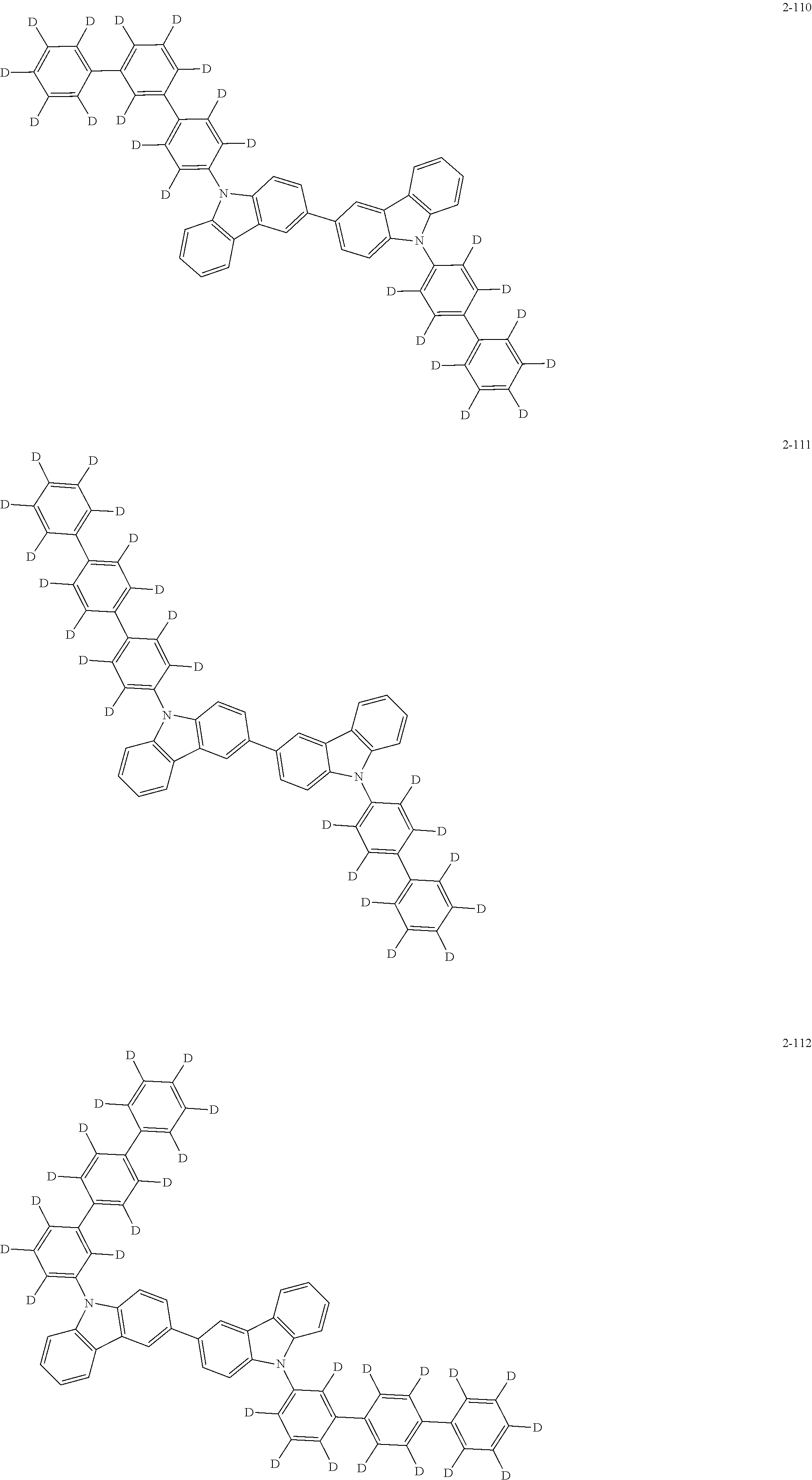

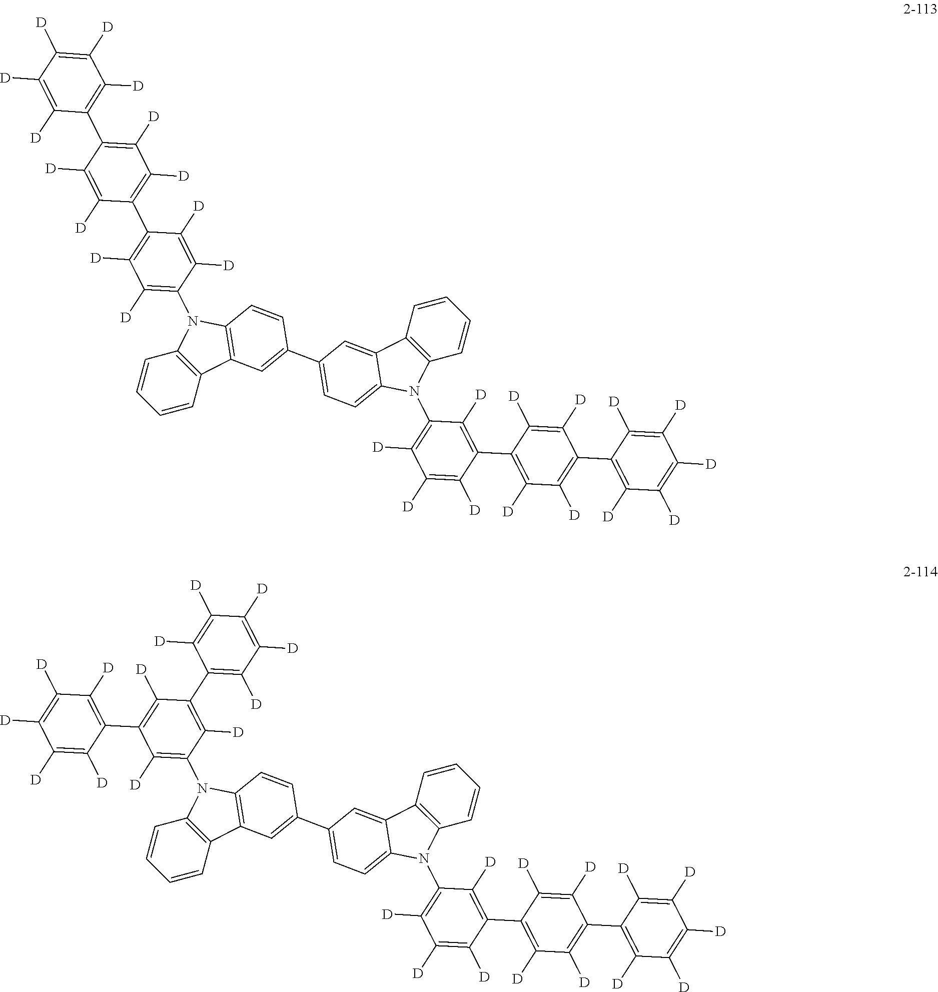

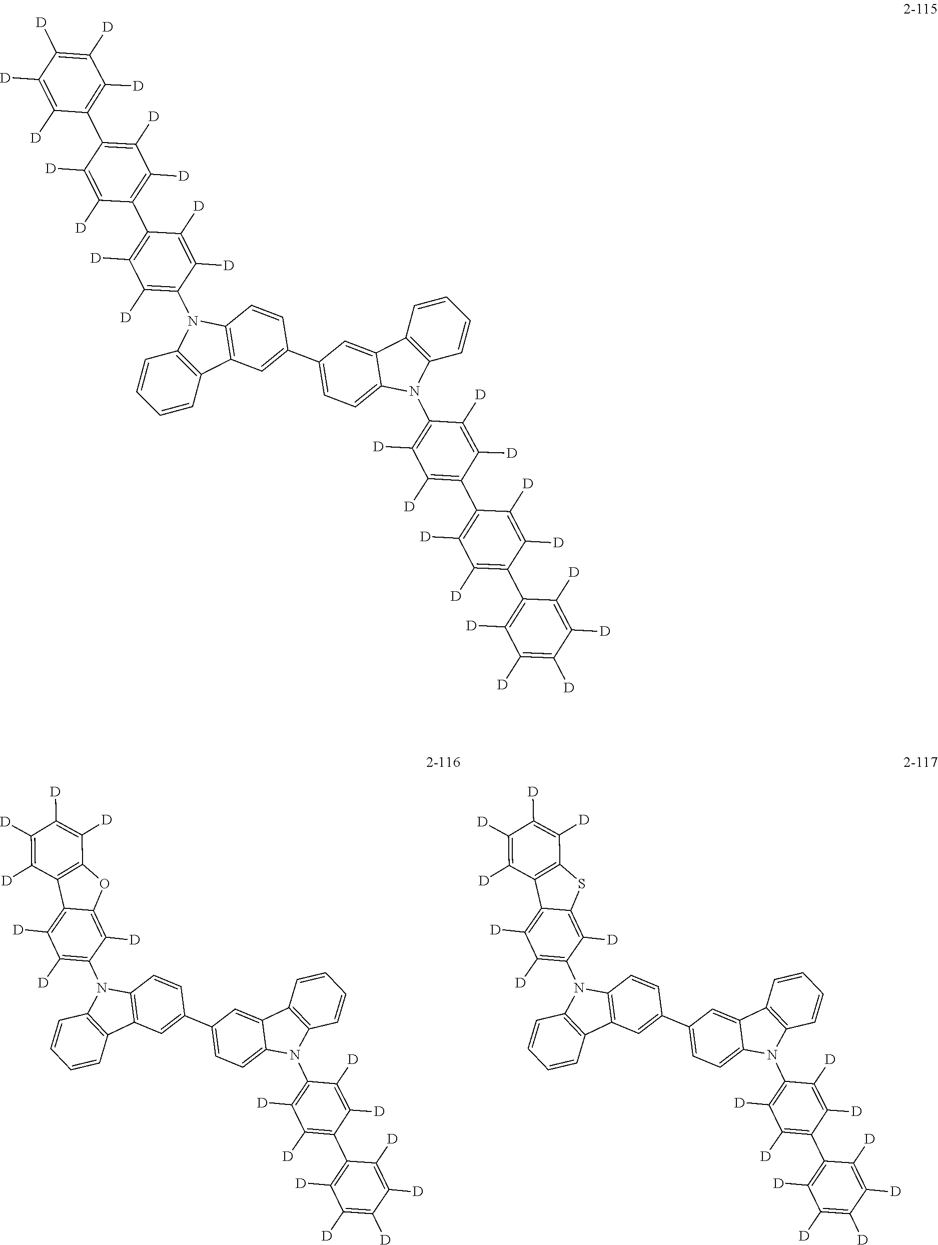

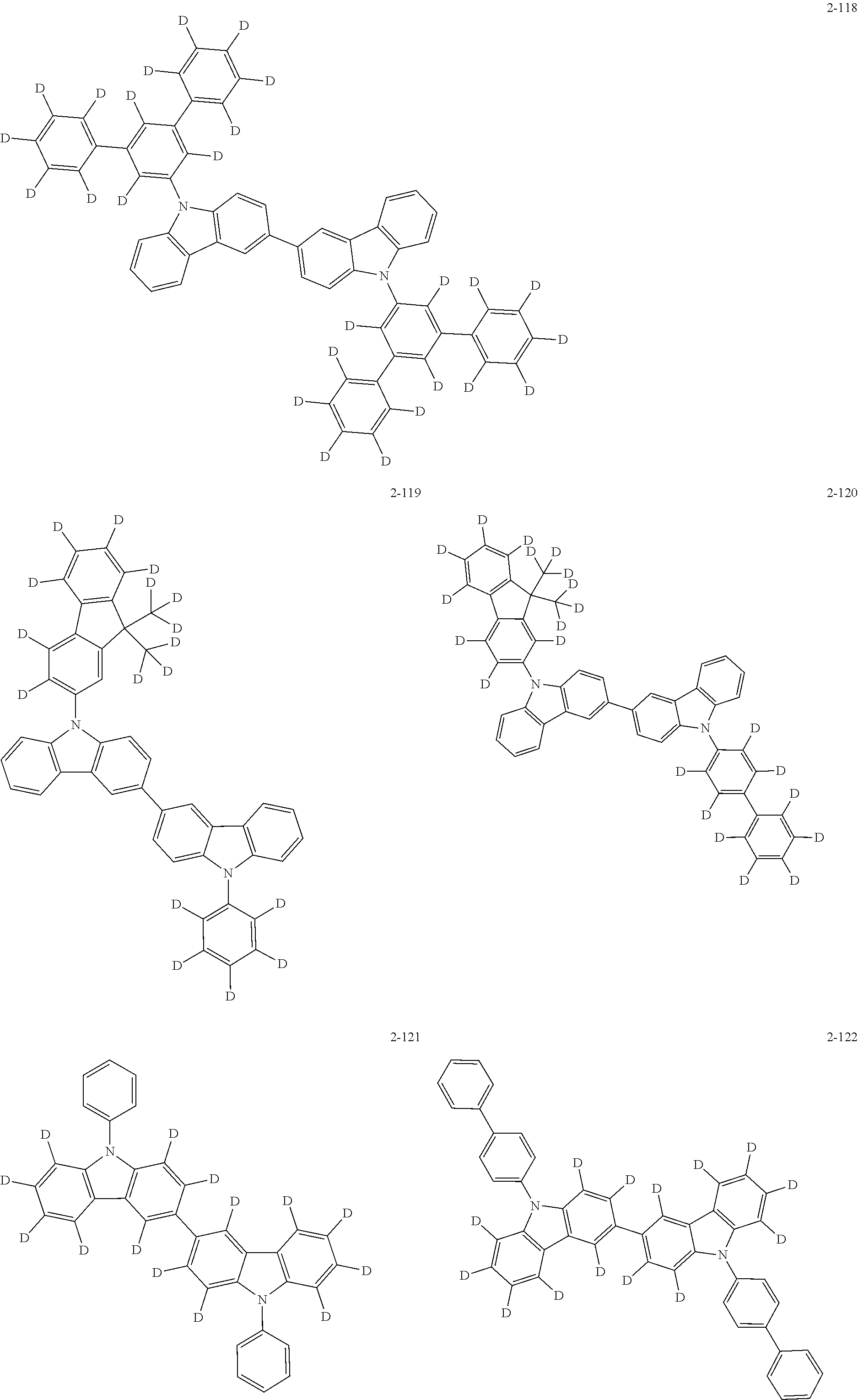

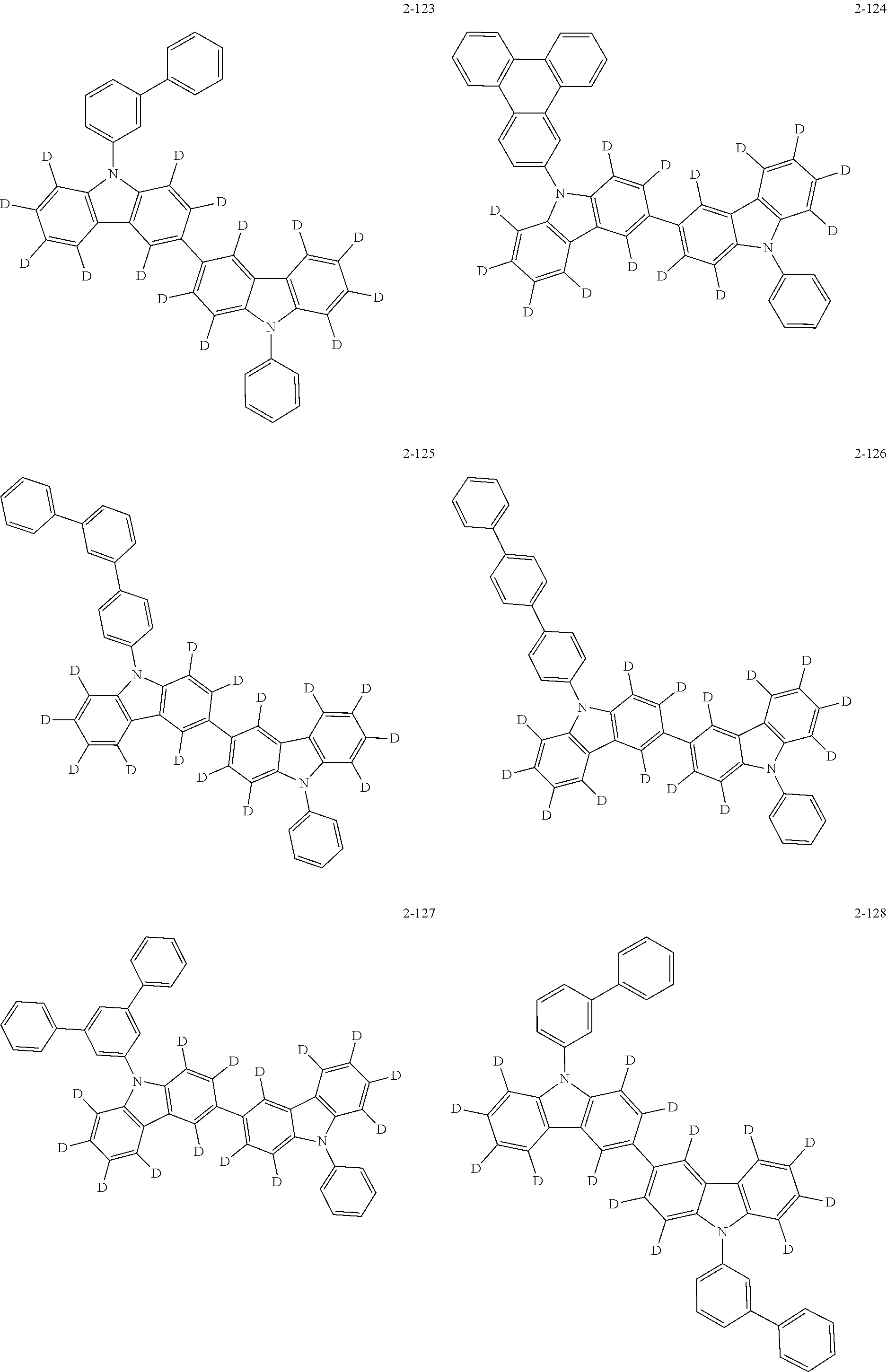

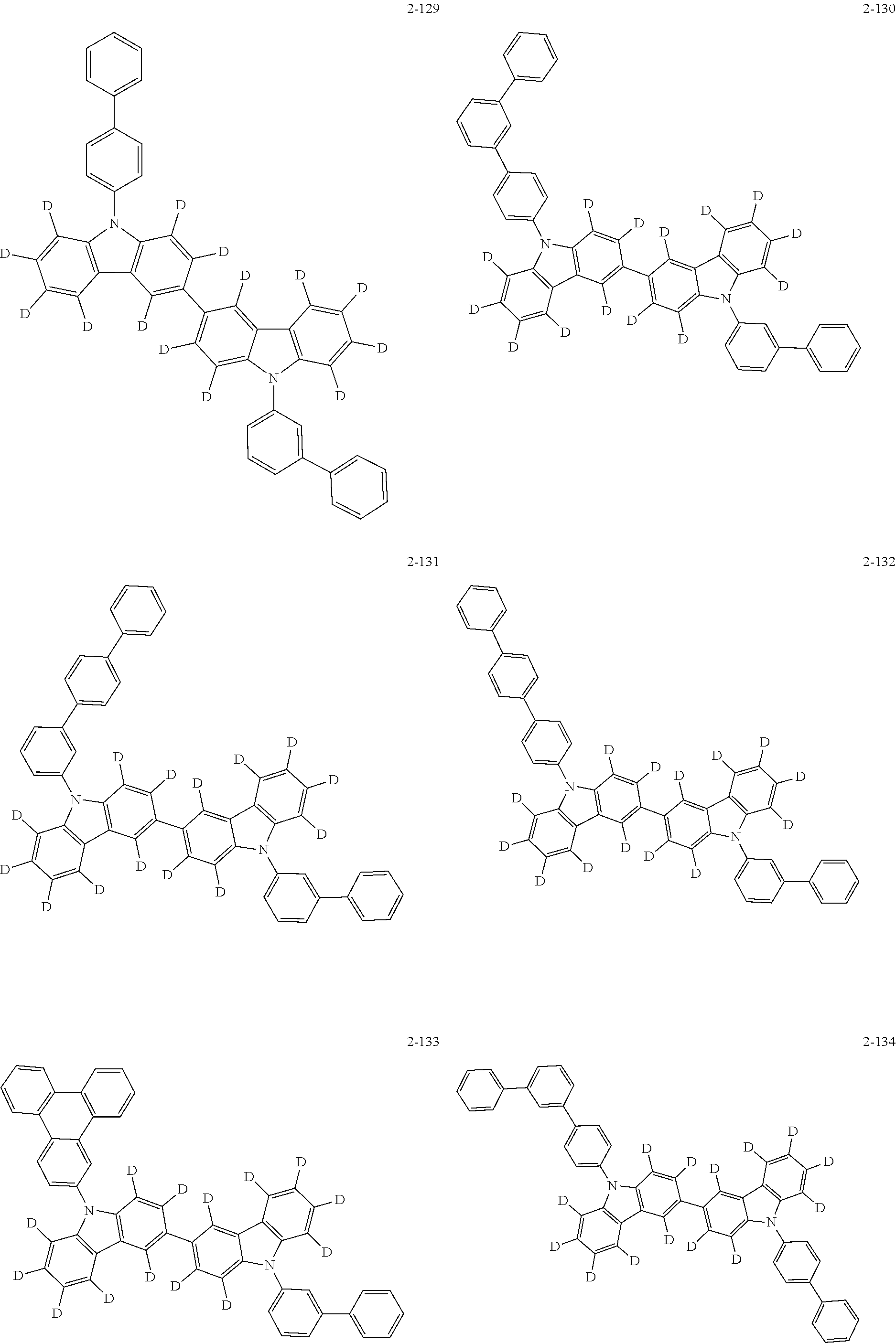

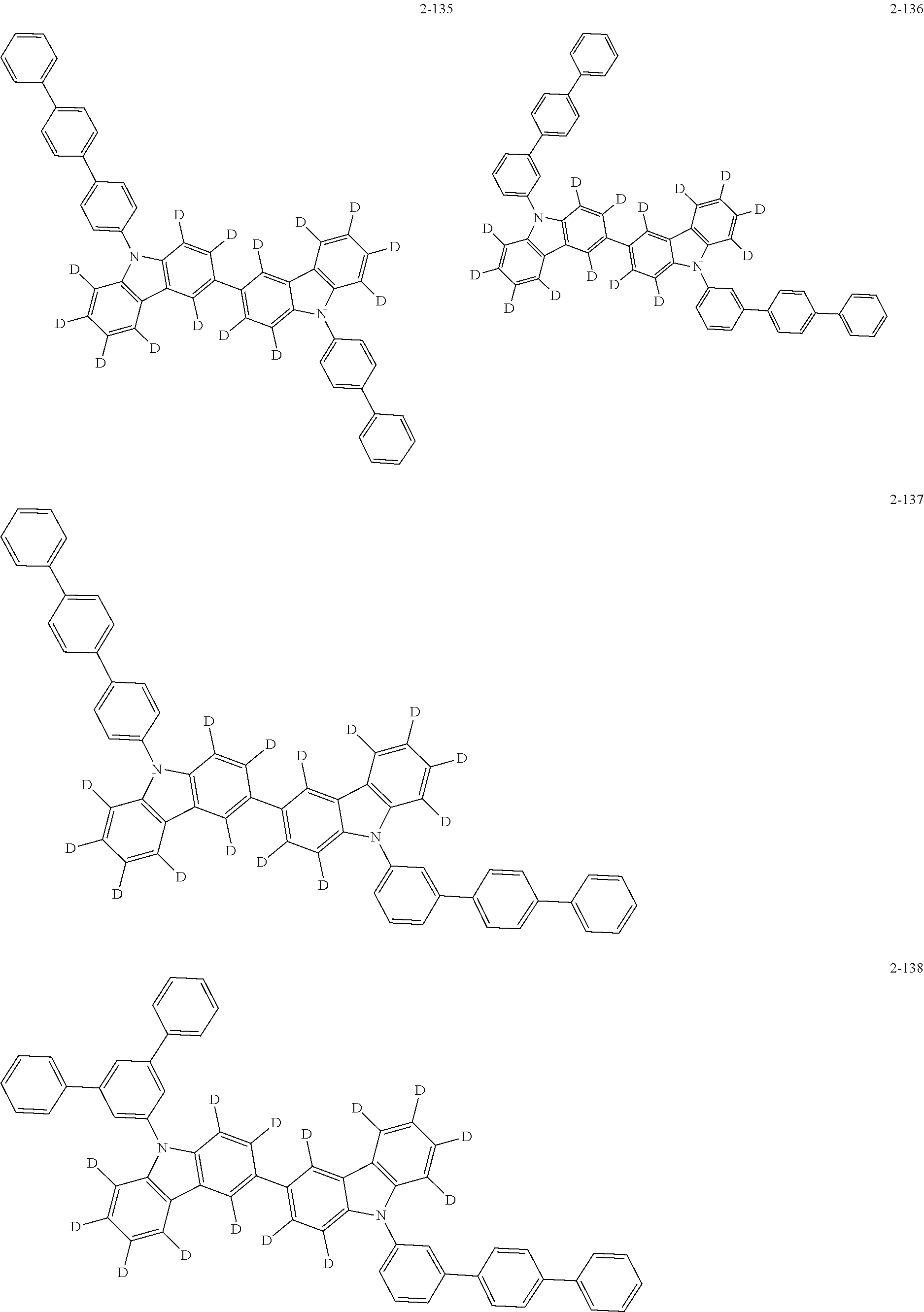

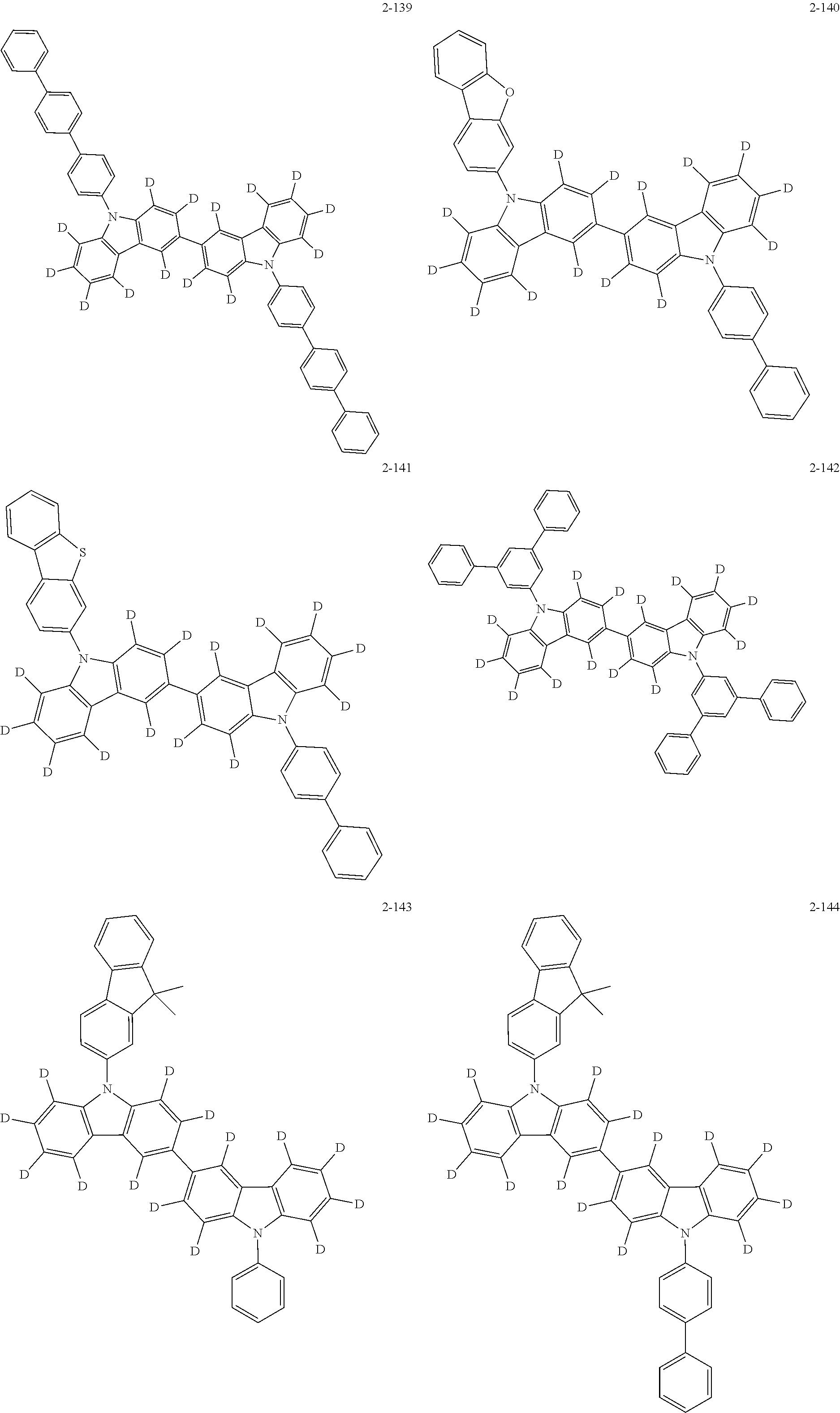

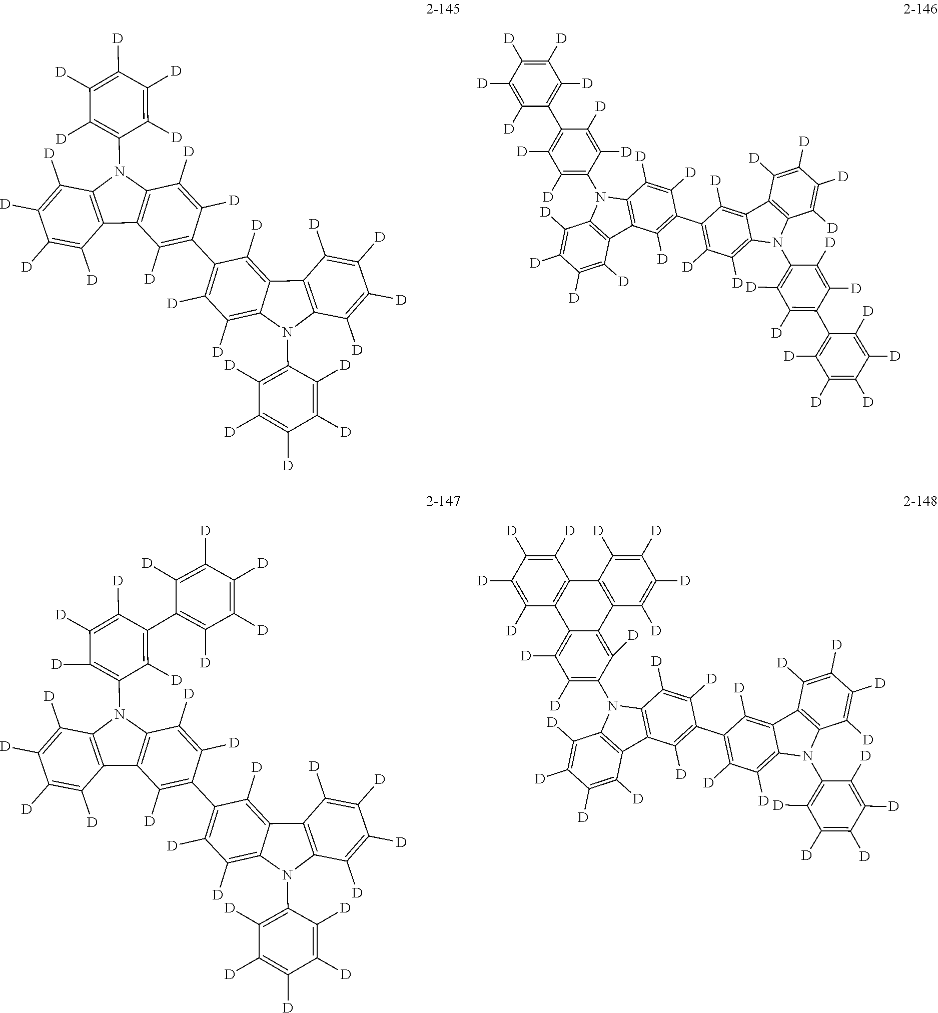

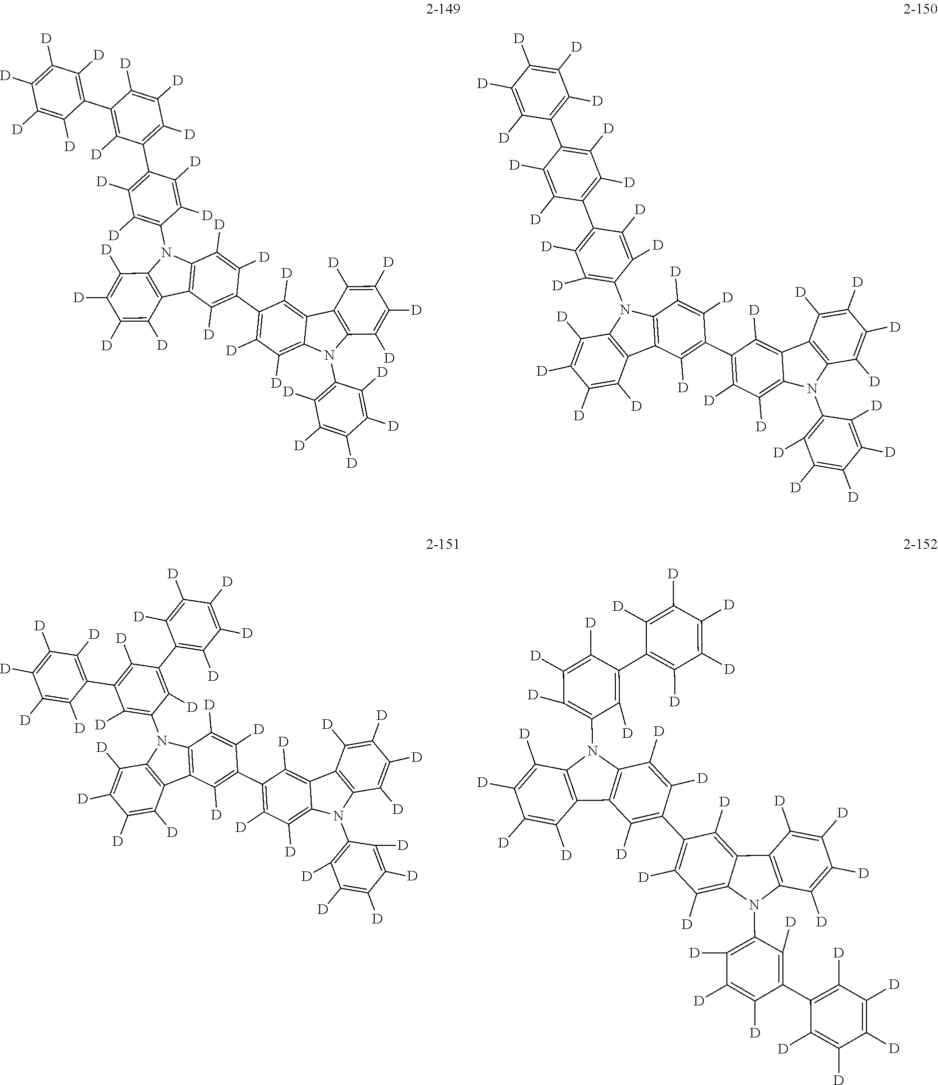

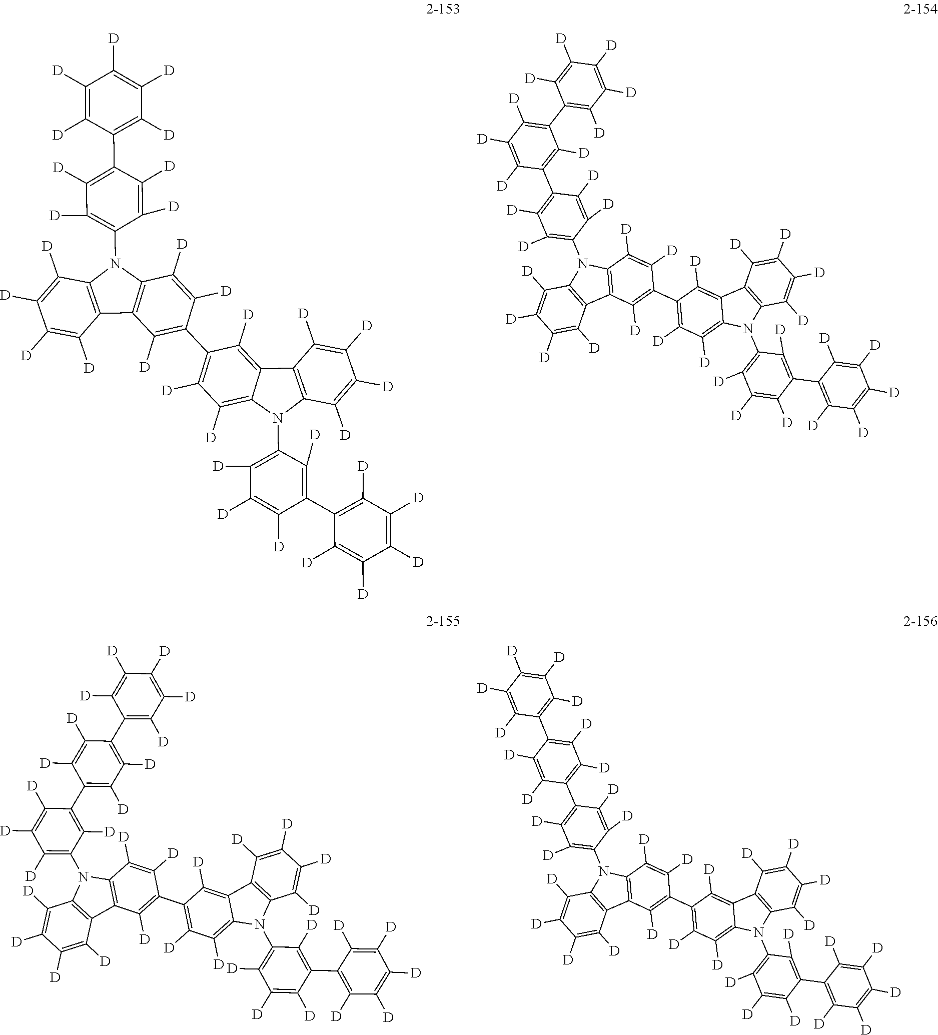

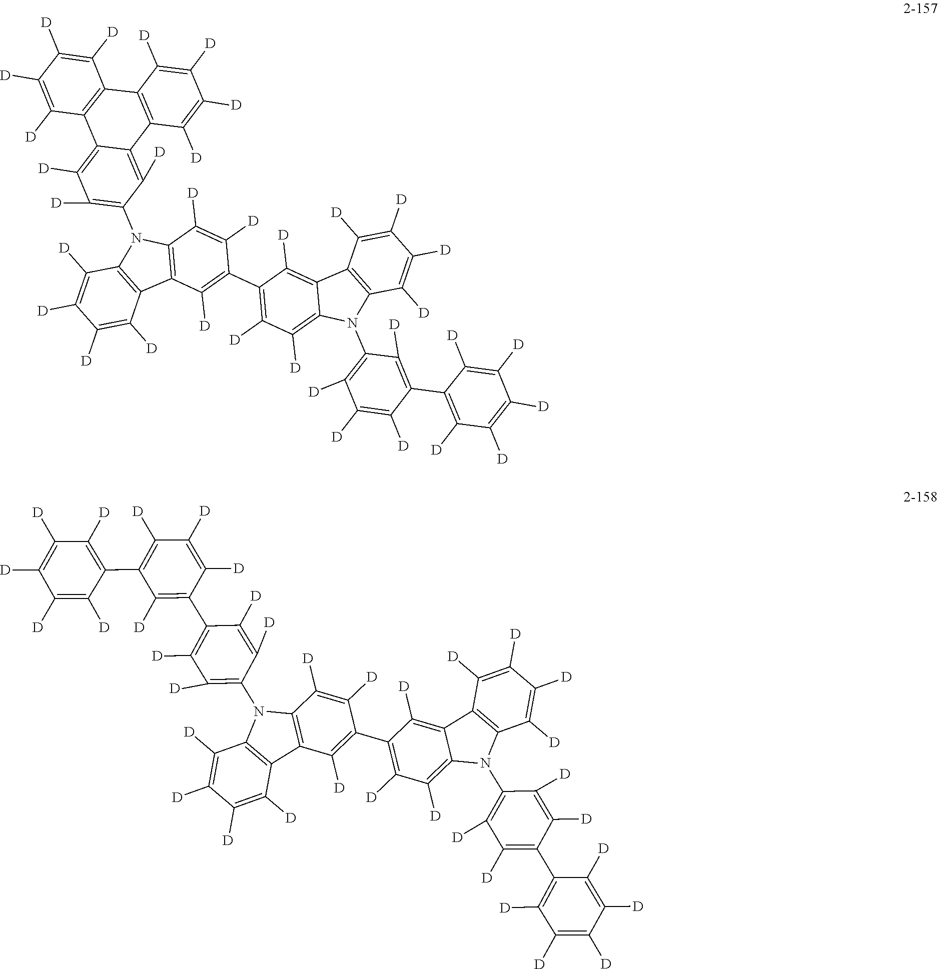

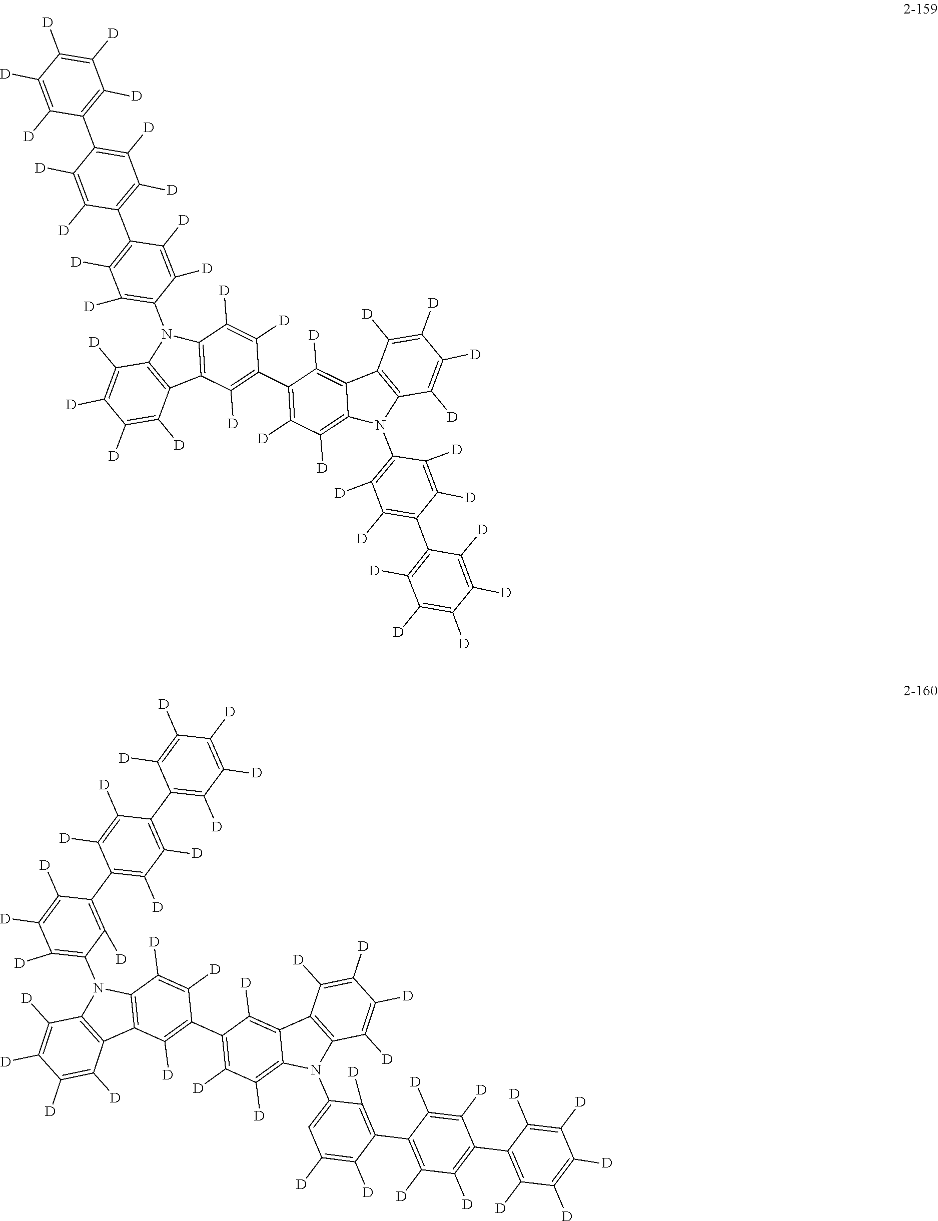

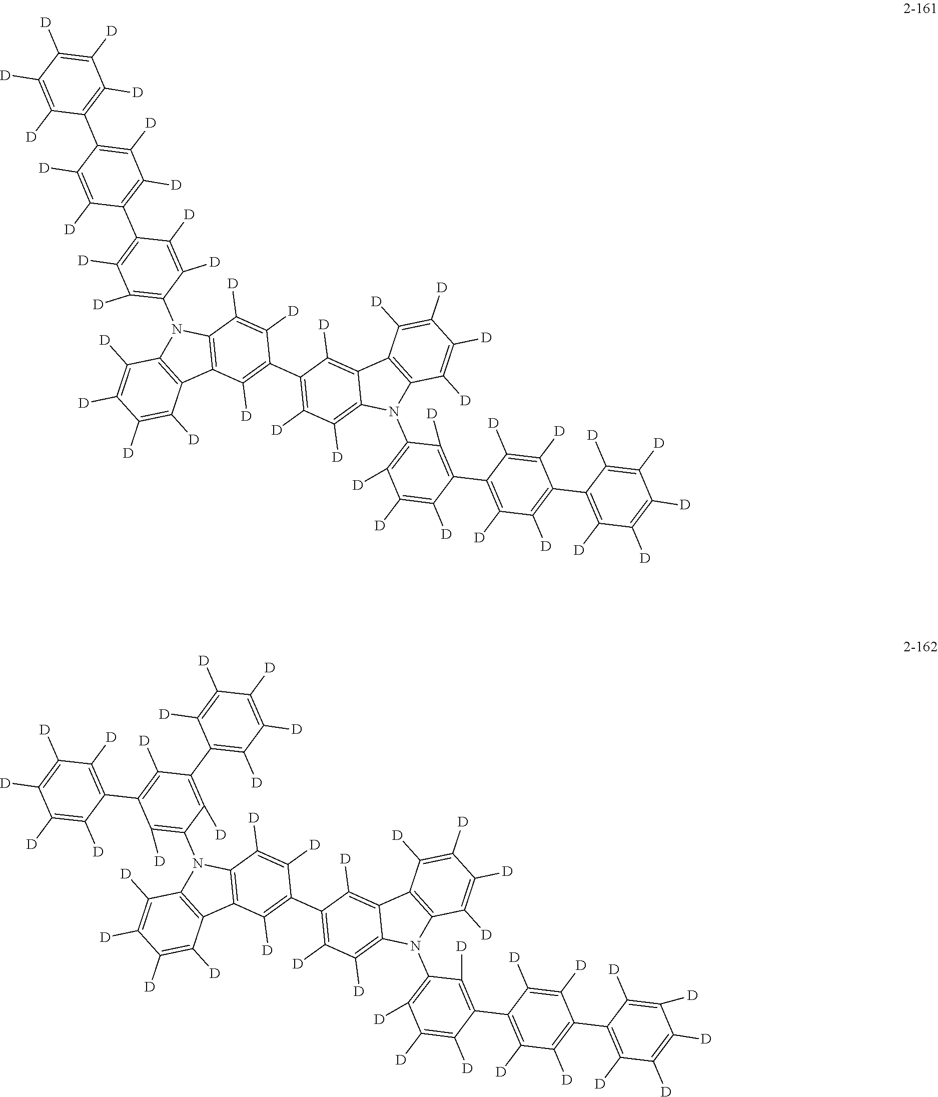

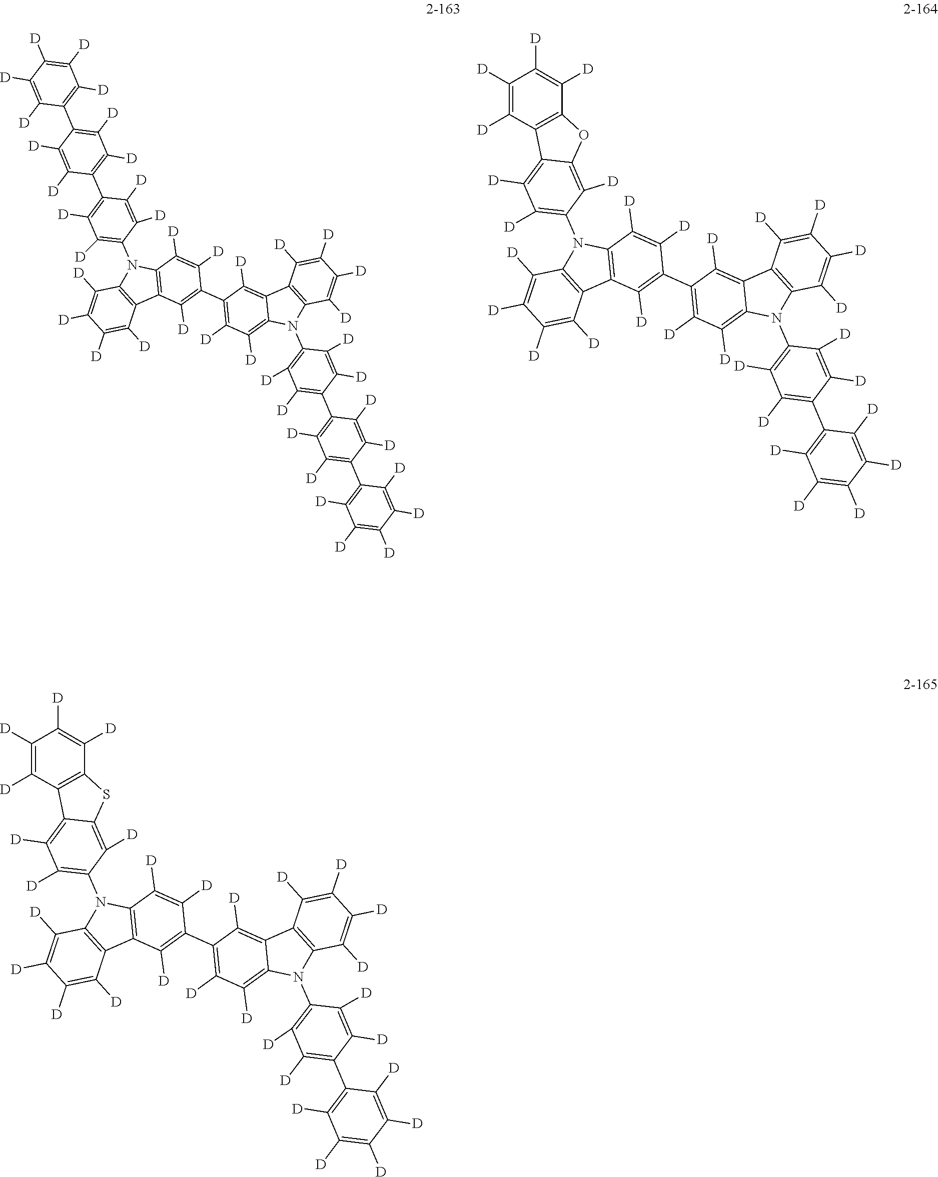

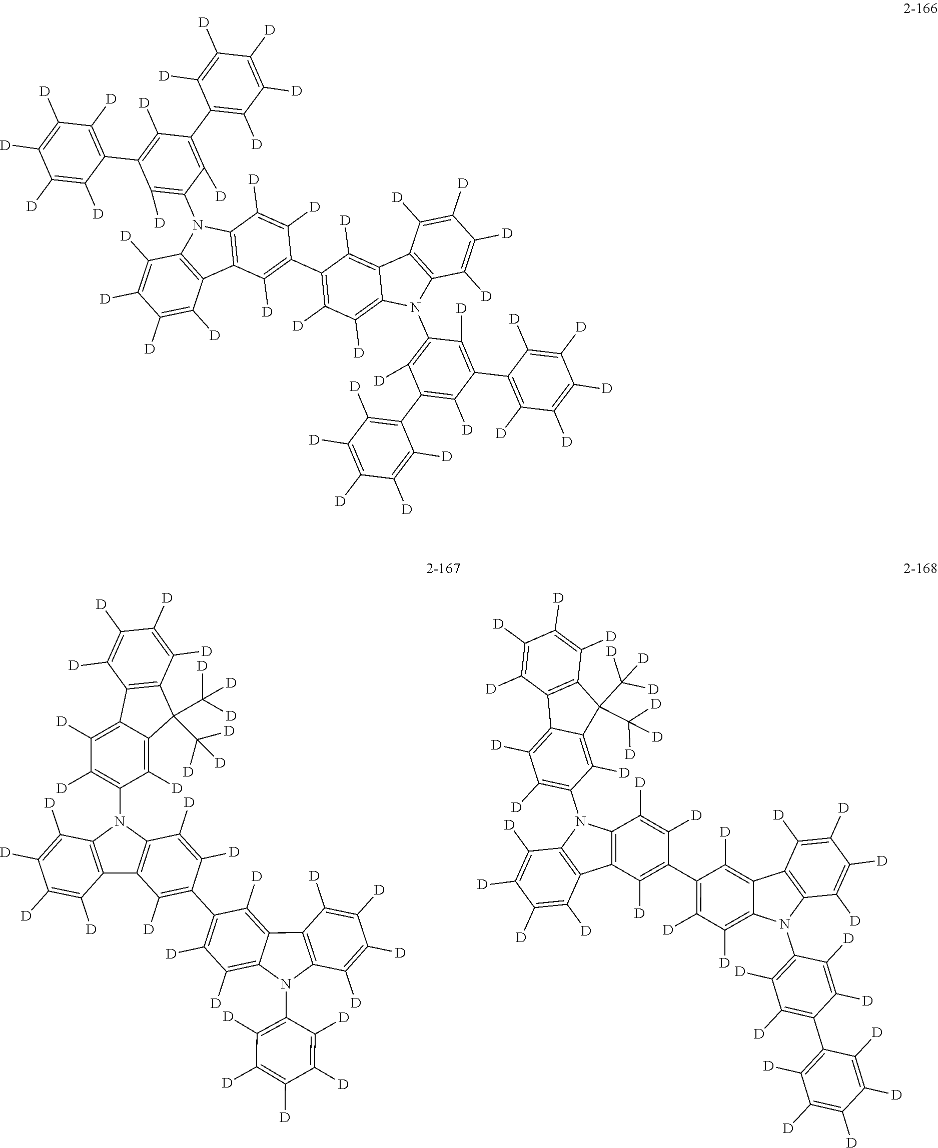

Further, another exemplary embodiment of the present application provides a composition for an organic material layer of an organic light emitting device, which includes the heterocyclic compound represented by Chemical Formula 1 and the heterocyclic compound represented by Chemical Formula 2.

The specific contents on the heterocyclic compound represented by Chemical Formula 1 and the heterocyclic compound represented by Chemical Formula 2 are the same as those described above.

The weight ratio of the heterocyclic compound represented by Chemical Formula 1: the heterocyclic compound represented by Chemical Formula 2 in the composition may be 1:10 to 10:1, 1:8 to 8:1, 1:5 to 5:1, and 1:2 to 2:1, but is not limited thereto.

The composition may be used when an organic material for an organic light emitting device is formed, and particularly, may be more preferably used when a host of a light emitting layer is formed.

The composition is in a form in which two or more compounds are simply mixed, materials in a powder state may also be mixed before an organic material layer of an organic light emitting device is formed, and it is possible to mix compounds in a liquid state at a temperature which is equal to or more than a suitable temperature. The composition is in a solid state at a temperature which is equal to or less than the melting point of each material, and may be maintained as a liquid phase when the temperature is adjusted.

The composition may additionally include materials publicly known in the art such as solvents and additives.

The organic light emitting device according to an exemplary embodiment of the present application may be manufactured by typical methods and materials for manufacturing an organic light emitting device, except that the organic material layer having one or more layers are formed by using the heterocyclic compound represented by Chemical Formula 1 and the heterocyclic compound represented by Chemical Formula 2, which are described above.

The compound represented by Chemical Formula 1 and the heterocyclic compound represented by Chemical Formula 2 may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present application, the organic light emitting device may be a blue organic light emitting device, and the heterocyclic compound according to Chemical Formula 1 and the heterocyclic compound according to Chemical Formula 2 may be used as a material for the blue organic light emitting device.

In an exemplary embodiment of the present application, the organic light emitting device may be a green organic light emitting device, and the compound represented by Chemical Formula 1 and the heterocyclic compound represented by Chemical Formula 2 may be used as a material for the green organic light emitting device.

In an exemplary embodiment of the present application, the organic light emitting device may be a red organic light emitting device, and the compound represented by Chemical Formula 1 and the heterocyclic compound represented by Chemical Formula 2 may be used as a material for the red organic light emitting device.

The organic light emitting device of the present invention may further include one or two or more layers selected from the group consisting of a light emitting layer, a hole injection layer, a hole transport layer, an electron injection layer, an electron transport layer, an electron blocking layer, and a hole blocking layer.

In an exemplary embodiment of the present application, provided is an organic light emitting device in which the organic material layer includes at least one layer of a hole blocking layer, an electron injection layer, and an electron transport layer, and at least one layer of the hole blocking layer, the electron injection layer, and the electron transport layer includes the heterocyclic compound represented by Chemical Formula 1 and the heterocyclic compound represented by Chemical Formula 2.

In an exemplary embodiment of the present application, provided is an organic light emitting device in which the organic material layer includes a light emitting layer, and the light emitting layer includes the heterocyclic compound represented by Chemical Formula 1 and the heterocyclic compound represented by Chemical Formula 2.

In an exemplary embodiment of the present application, provided is an organic light emitting device in which the organic material layer includes a light emitting layer, the light emitting layer includes a host material, and the host material includes the heterocyclic compound represented by Chemical Formula 1 and the heterocyclic compound represented by Chemical Formula 2.

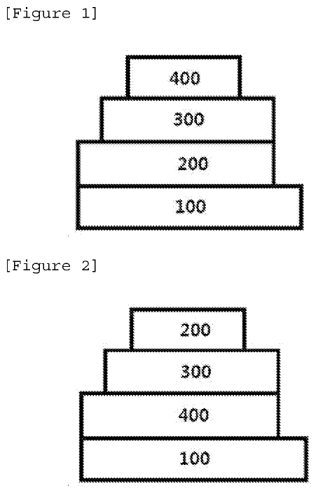

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

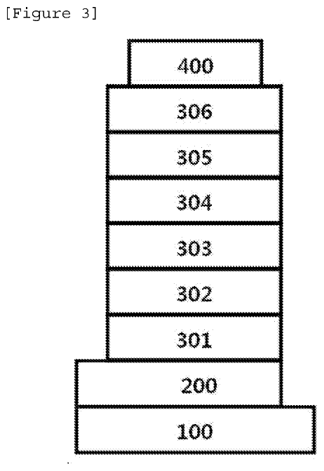

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

In an exemplary embodiment of the present application, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by pre-mixing the heterocyclic compound of Chemical Formula 1 and the heterocyclic compound of Chemical Formula 2, and using a thermal vacuum deposition method.

The pre-mixing means that before the heterocyclic compound of Chemical Formula 1 and the heterocyclic compound of Chemical Formula 2 are deposited onto an organic material layer, the materials are first mixed and the mixture is contained in one common container and mixed.

The pre-mixed material may be referred to as a composition for an organic material layer according to an exemplary embodiment of the present application.

In the organic light emitting device according to an exemplary embodiment of the present application, materials other than the heterocyclic compound of Chemical Formula 1 and the heterocyclic compound of Chemical Formula 2 will be exemplified below, but these materials are provided only for exemplification and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials may be deposited or used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

Hereinafter, the present specification will be described in more detail through Examples, but these Examples are provided only for exemplifying the present application, and are not intended to limit the scope of the present application.

### PREPARATION EXAMPLES

### <Preparation Example 1> Preparation of Compound 1-1

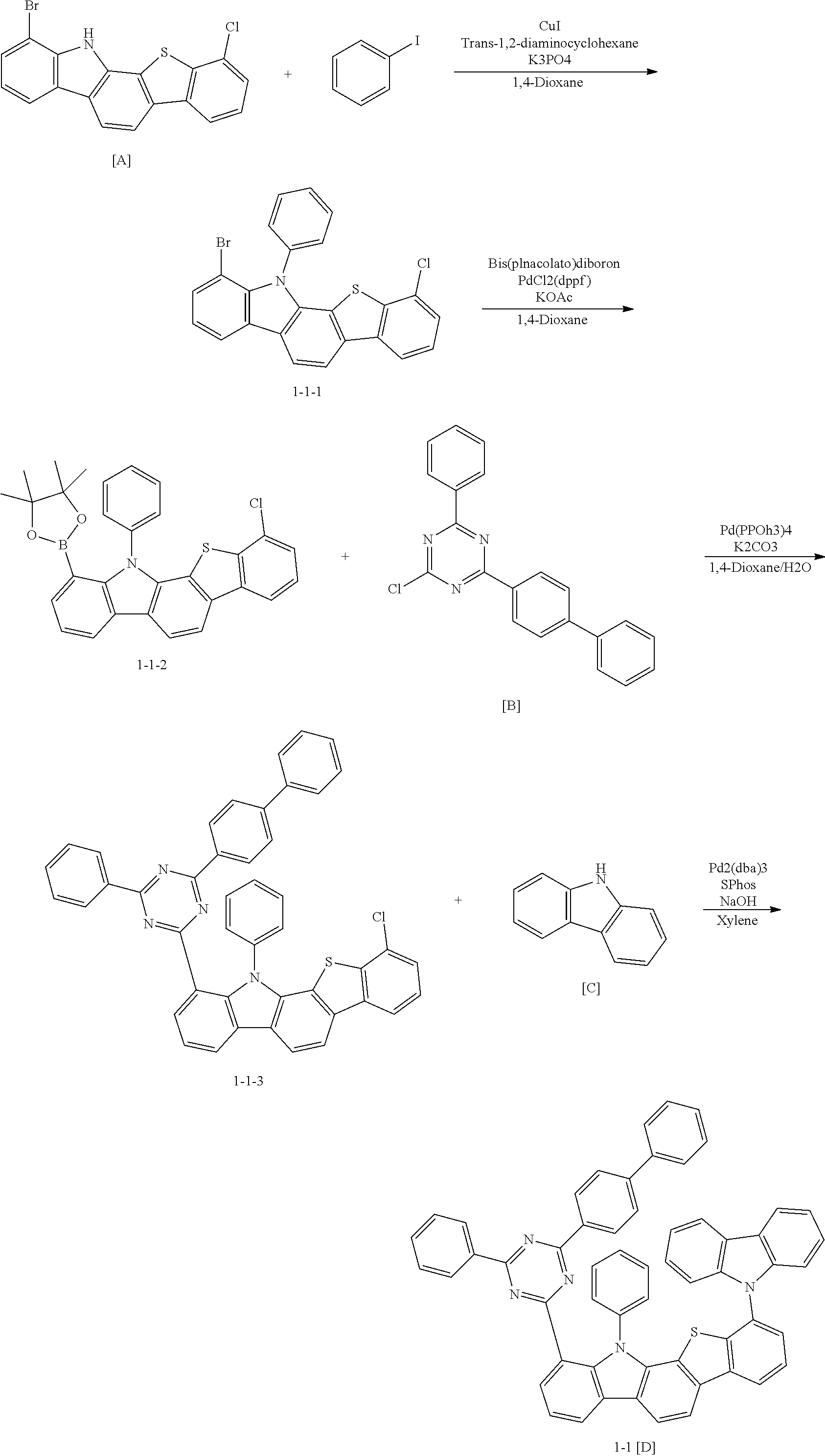

### 1) Preparation of Intermediate 1-1-1

A mixture of 1-bromo-10-chloro-12H-benzo[4,5]thieno[2,3-a]carbazole [A] (10 g, 0.026 mol), iodobenzene (6.33 g, 0.031 mol), CuI (4.95 g, 0.026 mol), trans-1,2-diaminocyclohexane (4.95 g, 0.026 mol), K₃Po₄ (11.04 g, 0.052 mol) and 1,4-dioxane (100 mL) was put into a one-neck round bottom flask and refluxed at 125° C. After the resulting product was extracted with DCM, concentrated, and then filtered with silica gel, the filtered product was concentrated was concentrated, and then treated with methanol to obtain target Compound 1-1-1. (11.07 g, yield 92%)

### 2) Preparation of Intermediate 1-1-2

A mixture of 1-bromo-10-chloro-12-phenyl-12H-benzo[4,5]thieno[2,3-a]carbazole (11.07 g, 0.024 mol), bis(pinacolato)diboron (12.19 g, 0.048 mol), Pd₂(dba)₃ (2.20 g, 0.0024 mol), Sphos (1.97 g, 0.0048 mol), potassium acetate (4.71 g, 0.048 mol) and 1,4-dioxane (110 mL) was refluxed at 120° C. in a one-neck round bottom flask. The resulting product was cooled, and then concentrated and filtered with silica gel to obtain Compound 1-1-2. (11.99 g, yield 98%)

### 3) Preparation of Intermediate 1-1-3

A mixture of 10-chloro-12-phenyl-1-(4,4,5,5-tetramethyl-1,3,2-dioxaborolan-2-yl)-12H-benzo[4,5]thieno[2,3-a]carbazole (11.99 g, 0.024 mol), 2-([1,1′-biphenyl]-4-yl)-4-chloro-6-phenyl-1,3,5-triazine (9.08 g, 0.026 mol), Pd(PPh₃)₄ (1.39 g, 0.0012 mol), K₂CO₃ (6.63 g, 0.048 mol) and 1,4-dioxane (100 mL)/water (30 mL) was refluxed at 120° C. in a one-neck round bottom flask. The resulting product was cooled, and then concentrated and filtered with silica gel to obtain Compound 1-1-3. (14.43 g, yield 87%)

### 4) Preparation of Compound 1-1[D]

A mixture of 1-(4-([1,1′-biphenyl]-4-yl)-6-phenyl-1,3,5-triazin-2-yl)-10-chloro-12-phenyl-12H-benzo[4,5]thieno[2,3-a]carbazole (14.43 g, 0.021 mol), 9H-carbazole (3.86 g, 0.023 mol), Pd₂(dba)₃ (1.92 g, 0.0021 mol), Sphos (1.97 g, 0.0048 mol), NaOH (1.68 g, 0.042 mol) and 1,4-dioxane (140 mL) was refluxed at 180° C. in a one-neck round bottom flask. The resulting product was cooled, and then concentrated and filtered with silica gel to obtain Compound 1-1[D]. (14.67 g, yield 85%)

Target compounds were prepared by performing preparation in the same manner as in Preparation Example 1, except that Compounds A to C of the following Table 1 were used instead of Compounds A to C in Preparation Example 1.

TABLE 1

| Com- | | | | | |
|---|---|---|---|---|---|
| pound | A | B | C | D | Yield |
| 1-1 | 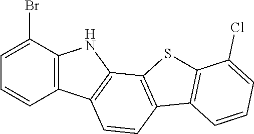 | 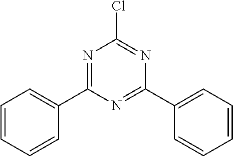 | 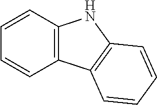 | 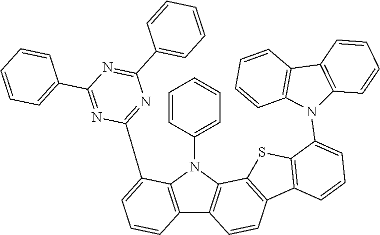 | 85% |
| 1-5 | 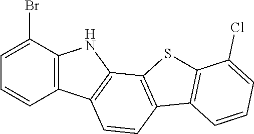 | 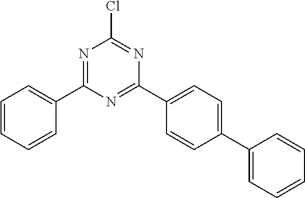 | 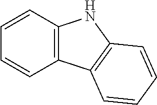 | 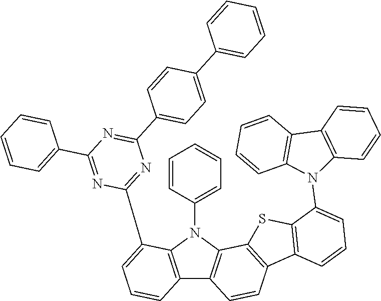 | 67% |
| 1-12 | 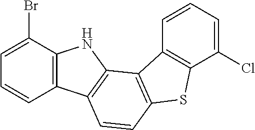 | 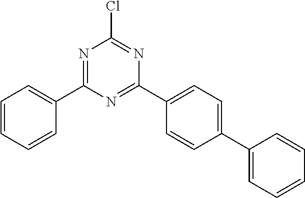 | 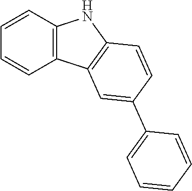 | 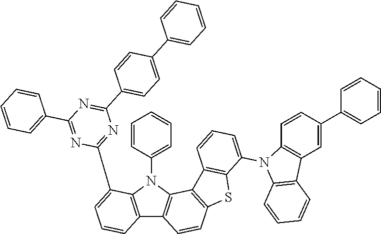 | 83% |
| 1-22 | 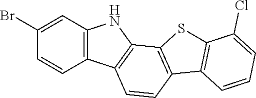 | 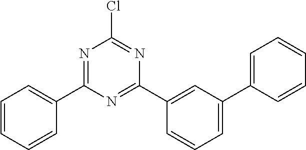 | 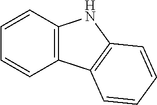 | 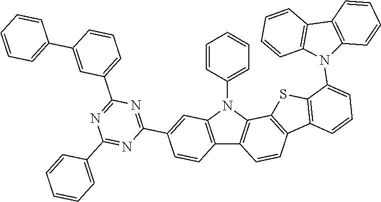 | 74% |
| 1-32 | 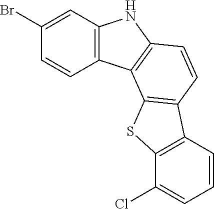 | 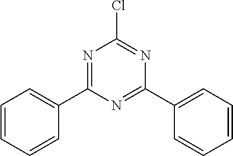 | 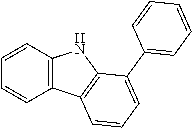 | 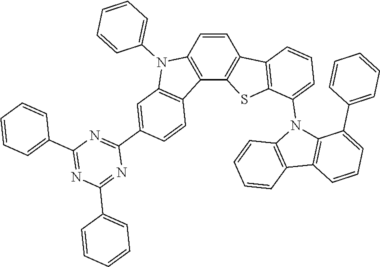 | 68% |
| 1-38 | 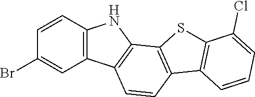 | 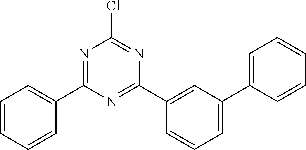 | 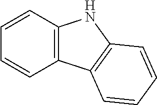 | 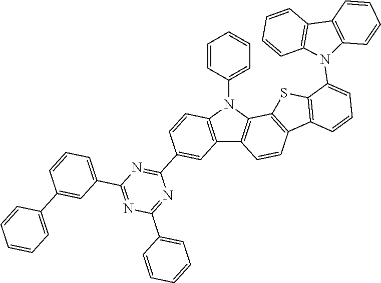 | 83% |
| 1-42 | 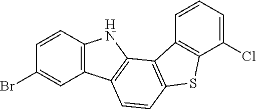 | 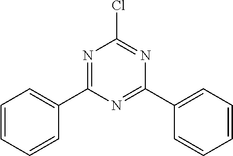 | 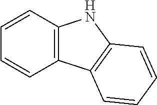 | 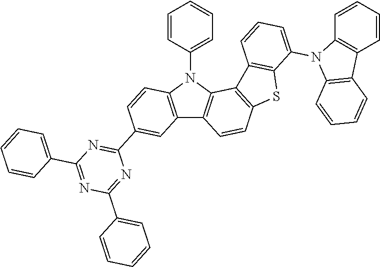 | 87% |
| 1-46 | 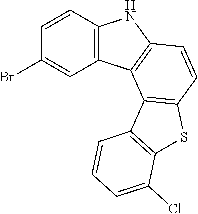 | 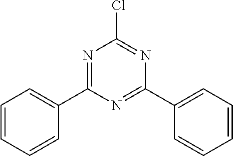 | 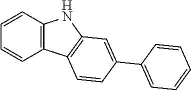 | 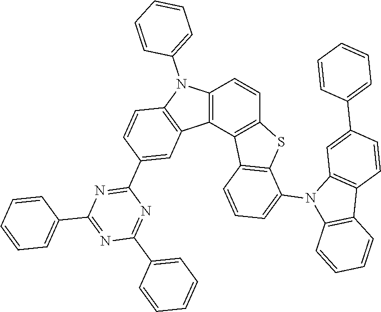 | 66% |
| 1-50 | 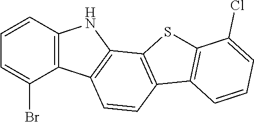 | 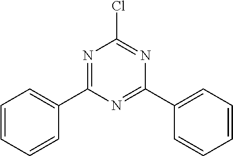 | 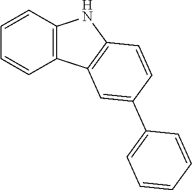 | 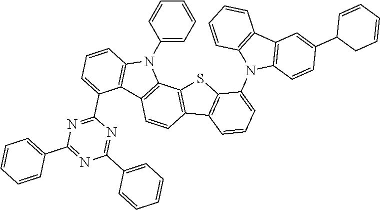 | 53% |
| 1-53 | 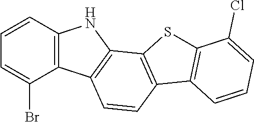 | 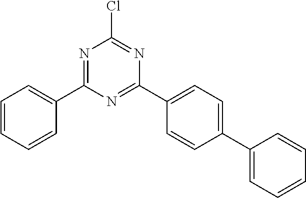 | 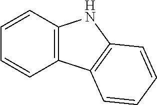 | 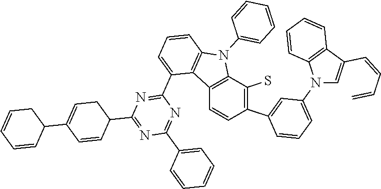 | 74% |
| 1-60 | 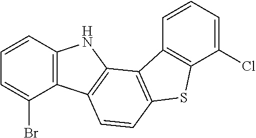 | 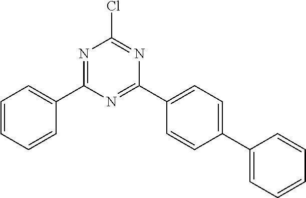 | 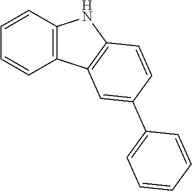 | 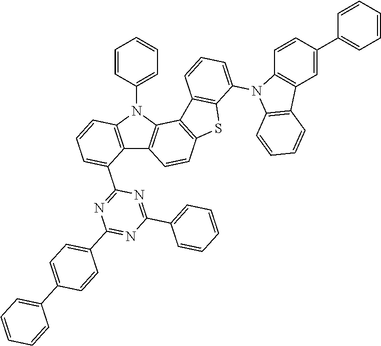 | 69% |
| 1-63 | 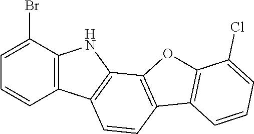 | 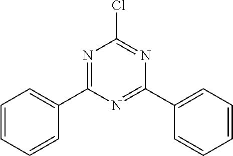 | 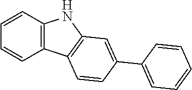 | 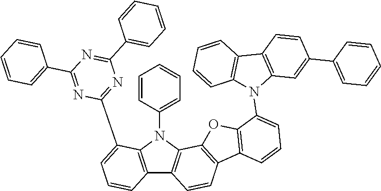 | 71% |
| 1-65 | 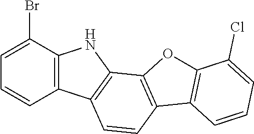 | 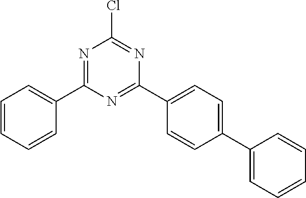 | 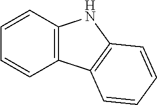 | 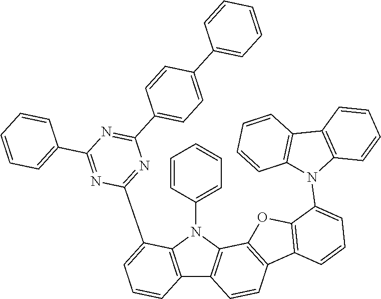 | 66% |
| 1-72 | 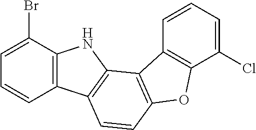 | 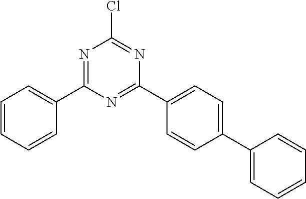 | 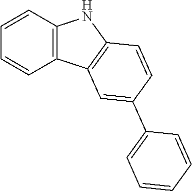 | 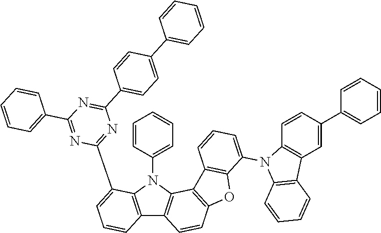 | 54% |
| 1-79 | 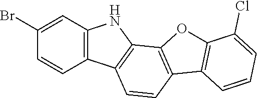 | 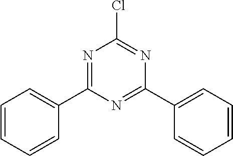 | 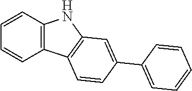 | 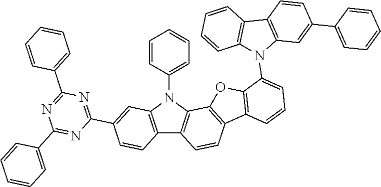 | 74% |
| 1-81 | 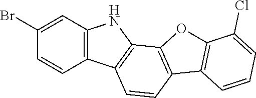 | 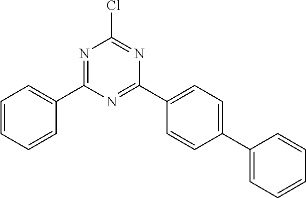 | 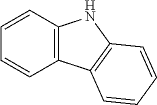 | 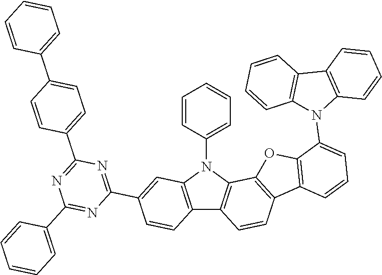 | 68% |
| 1-88 | 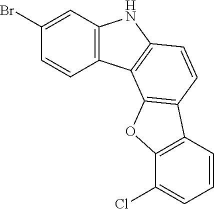 | 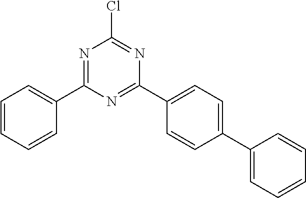 | 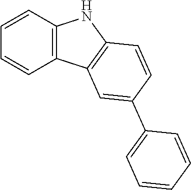 | 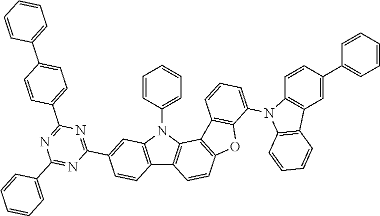 | 73% |
| 1-91 | 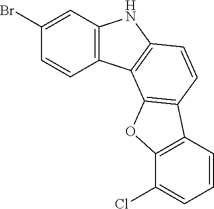 | 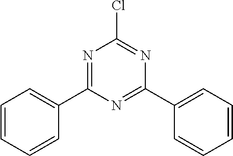 | 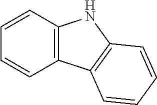 | 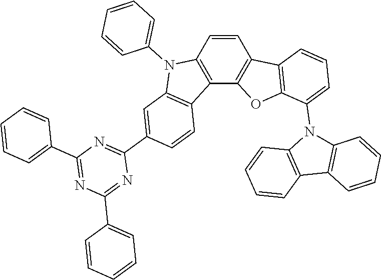 | 83% |
| 1-97 | 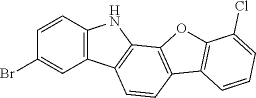 | 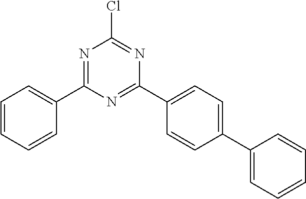 | 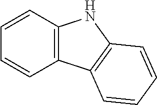 | 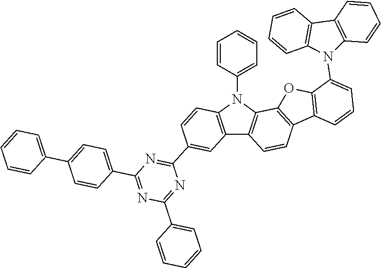 | 68% |
| 1-100 | 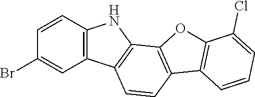 | 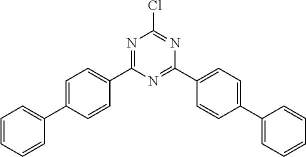 | 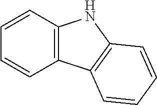 | 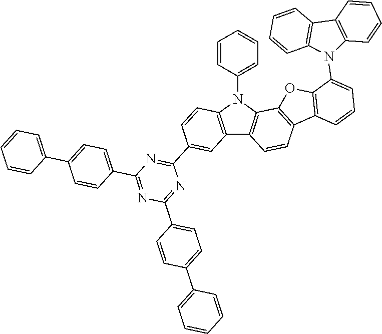 | 77% |
| 1-102 | 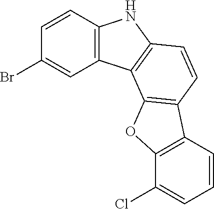 | 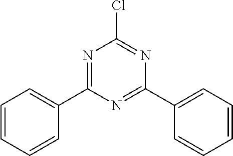 | 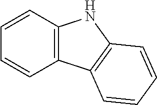 | 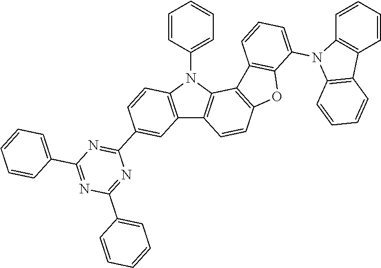 | 72% |
| 1-108 | 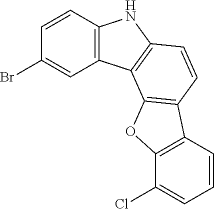 | 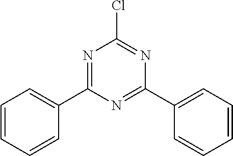 | 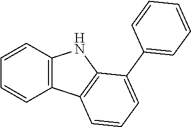 | 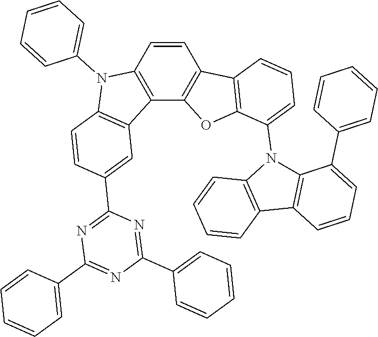 | 69% |
| 1-113 | 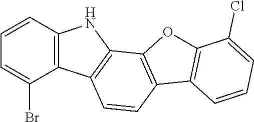 | 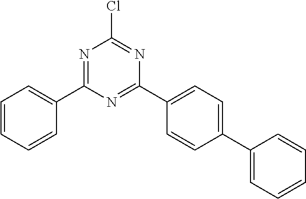 | 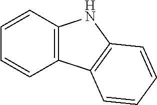 | 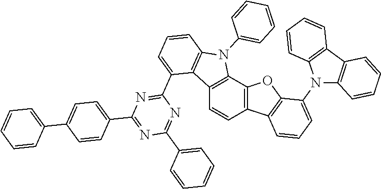 | 83% |
| 1-120 | 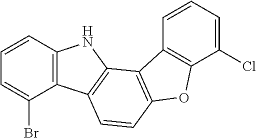 | 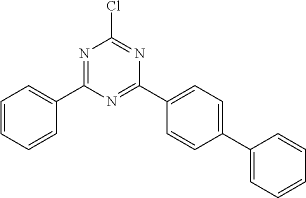 | 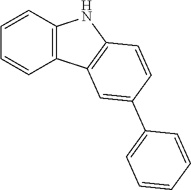 | 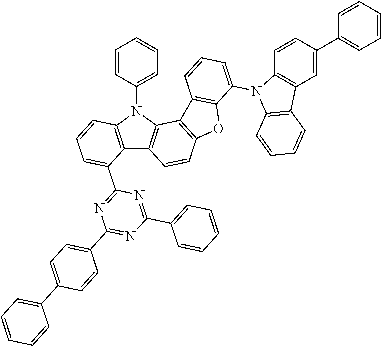 | 80% |
| 1-125 | 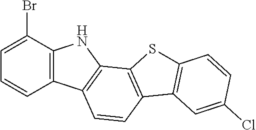 | 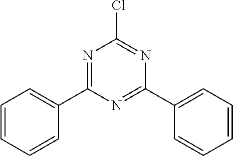 | 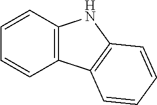 | 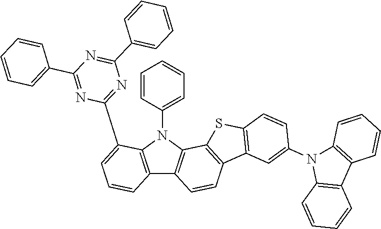 | 79% |
| 1-126 | 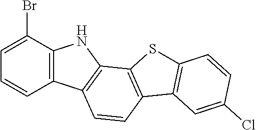 | 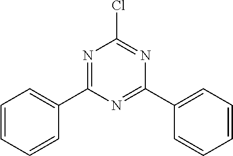 | 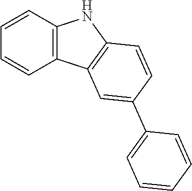 | 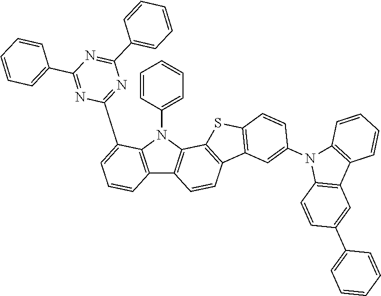 | 74% |
| 1-129 | 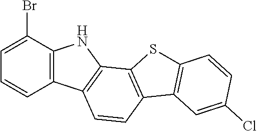 | 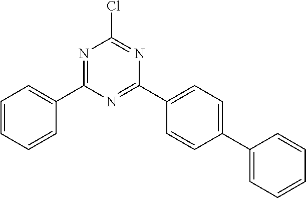 | 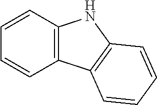 | 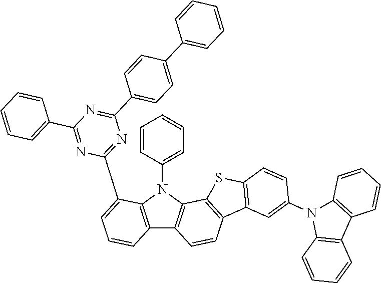 | 65% |
| 1-132 | 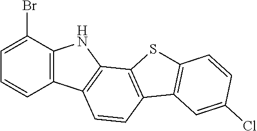 | 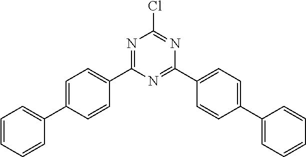 | 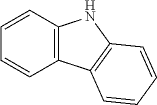 | 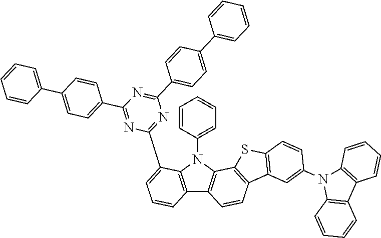 | 61% |
| 1-141 | 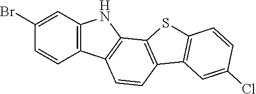 | 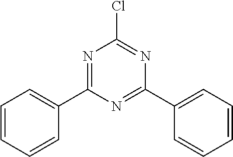 | 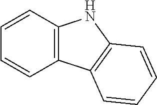 | 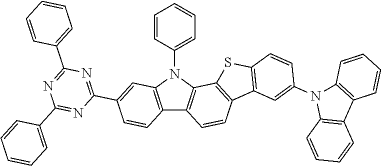 | 79% |
| 1-143 | 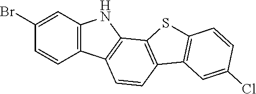 | 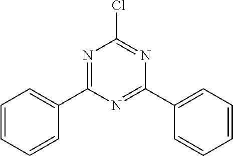 | 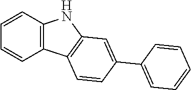 | 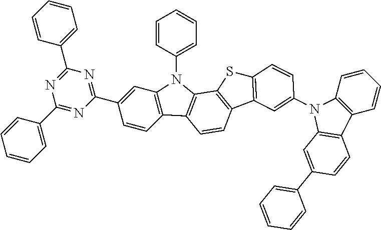 | 88% |
| 1-145 | 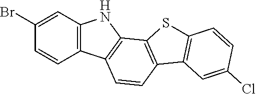 | 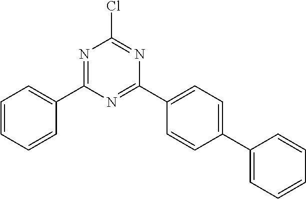 | 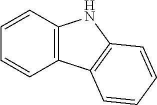 | 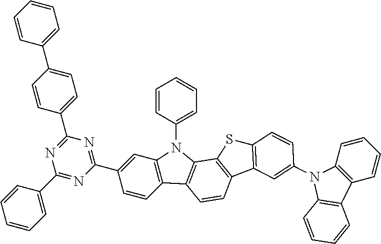 | 93% |
| 1-150 | 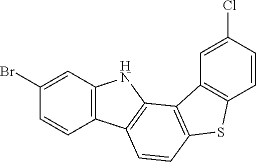 | 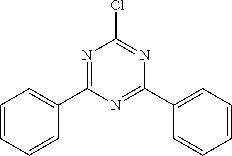 | 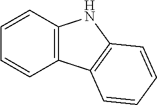 | 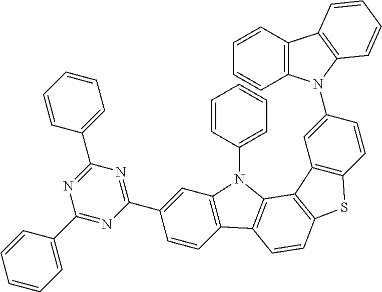 | 51% |
| 1-154 | 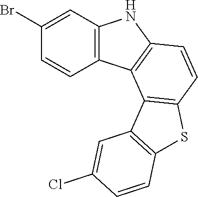 | 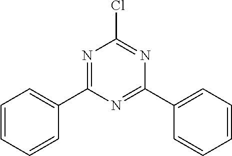 | 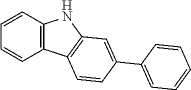 | 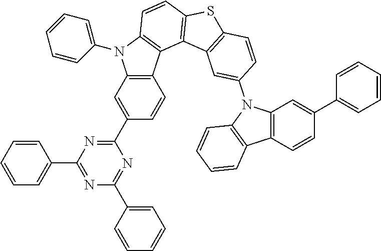 | 63% |
| 1-158 | 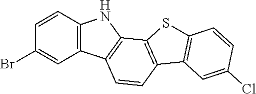 | 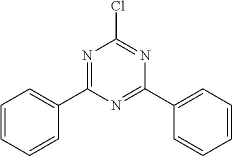 | 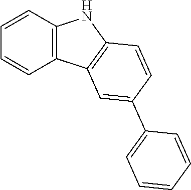 | 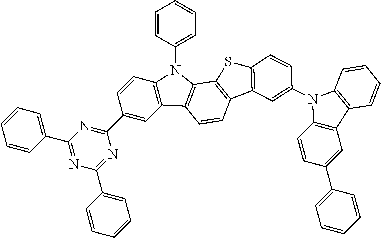 | 79% |
| 1-173 | 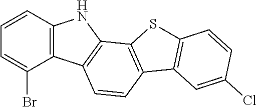 | 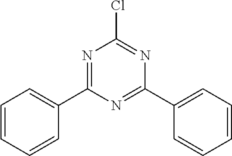 | 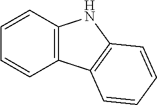 | 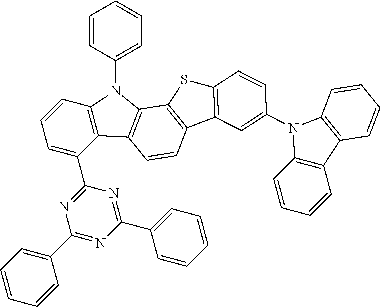 | 55% |
| 1-177 | 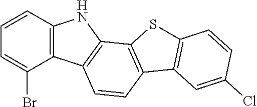 | 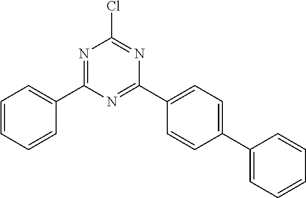 | 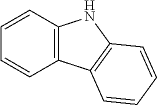 | 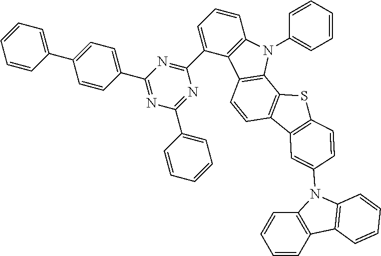 | 76% |
| 1-182 | 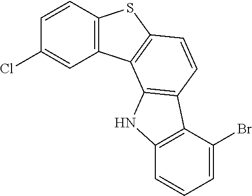 | 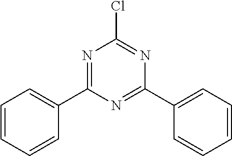 | 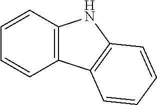 | 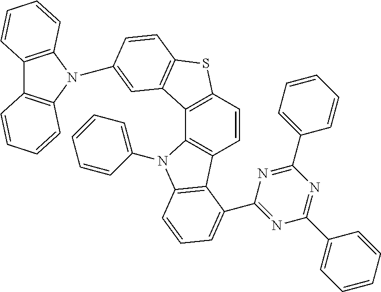 | 84% |
| 1-185 | 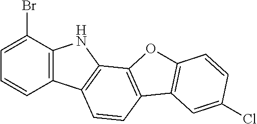 | 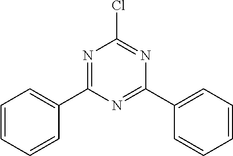 | 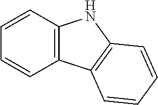 | 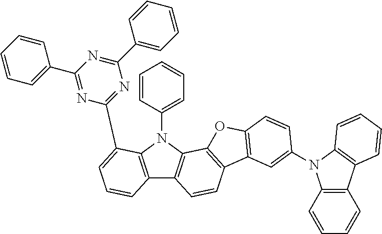 | 65% |
| 1-189 | 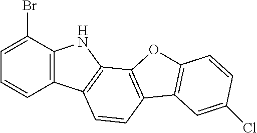 | 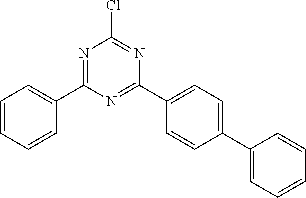 | 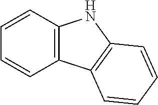 | 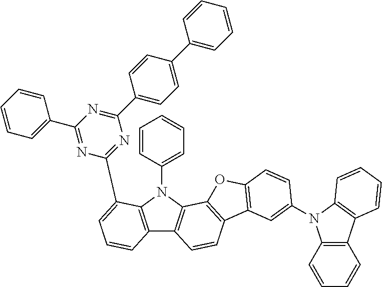 | 79% |
| 1-199 | 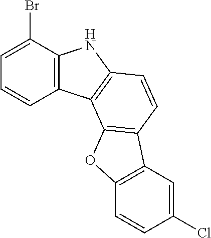 | 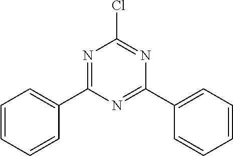 | 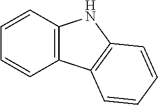 | 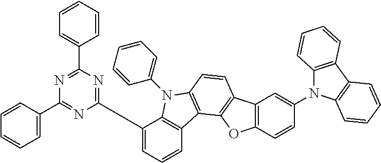 | 83% |
| 1-201 | 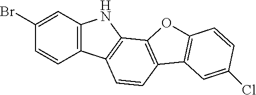 | 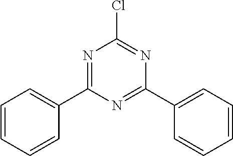 | 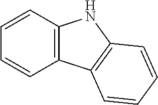 | 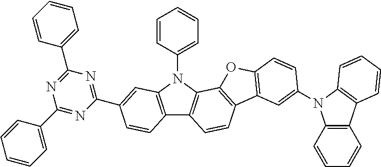 | 74% |
| 1-203 | 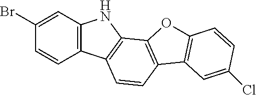 | 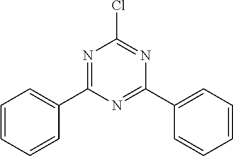 | 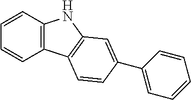 | 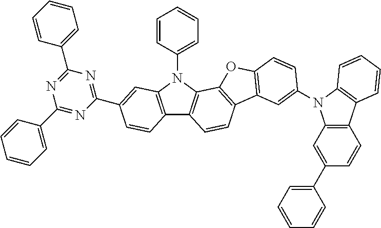 | 82% |
| 1-212 | 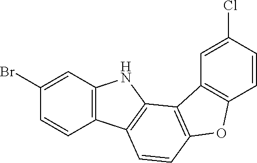 | 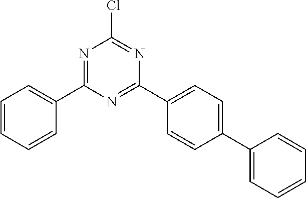 | 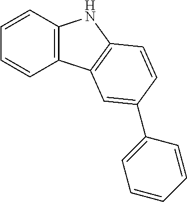 | 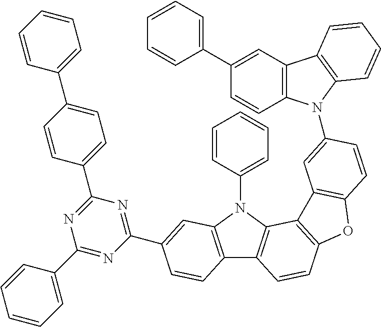 | 769 |
| 1-214 | 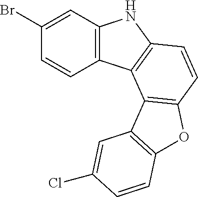 | 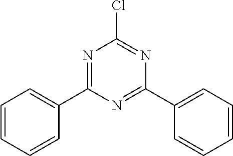 | 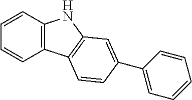 | 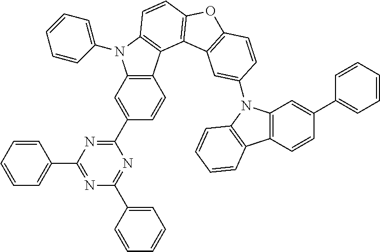 | 83% |
| 1-218 | 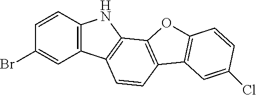 | 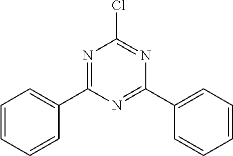 | 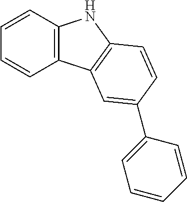 | 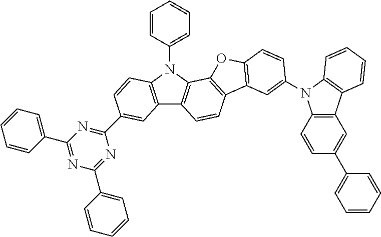 | 74% |
| 1-221 | 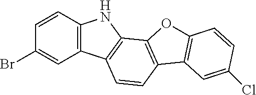 | 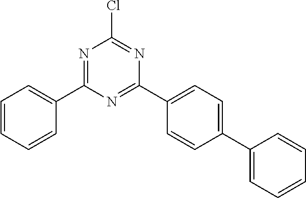 | 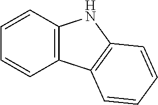 | 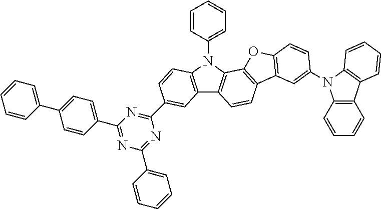 | 69% |
| 1-249 | 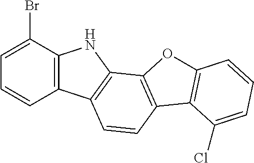 | 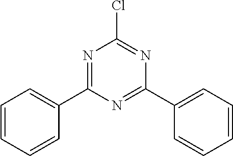 | 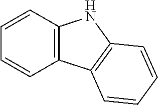 | 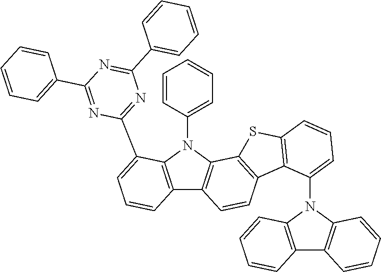 | 59% |
| 1-253 | 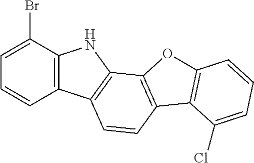 | 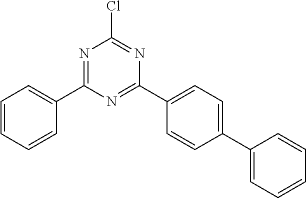 | 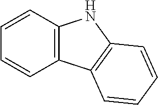 | 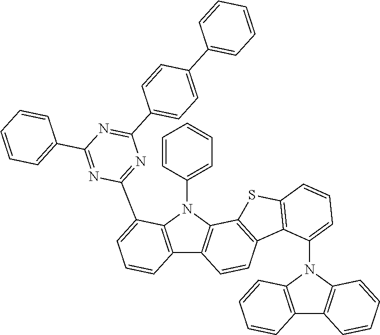 | 61% |
| 1-257 | 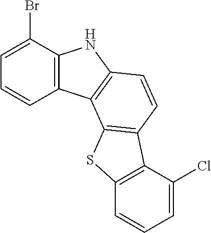 | 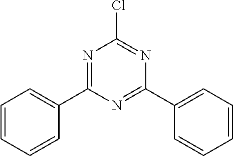 | 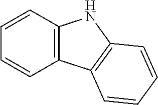 | 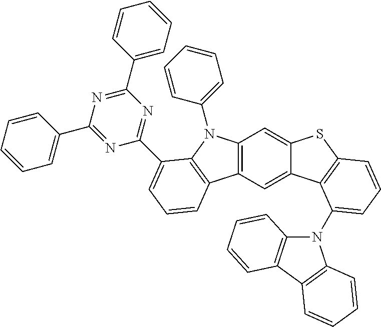 | 72% |
| 1-261 | 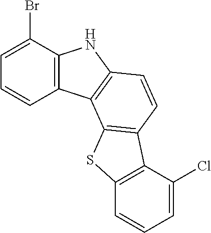 | 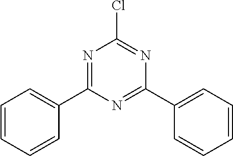 | 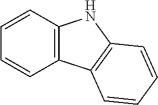 | 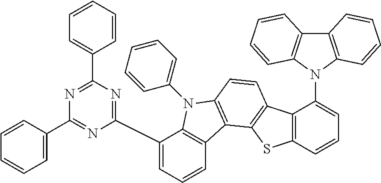 | 64% |
| 1-263 | 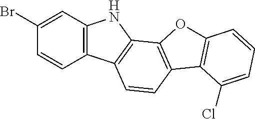 | 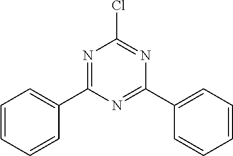 | 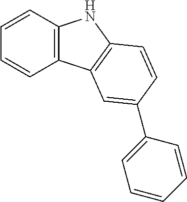 | 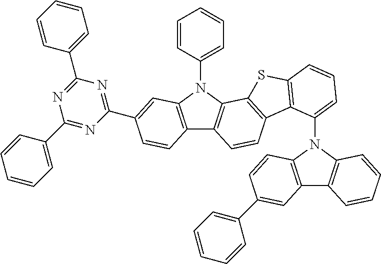 | 53% |
| 1-265 | 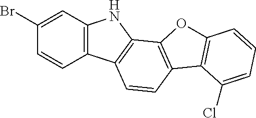 | 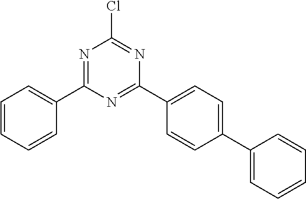 | 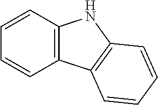 | 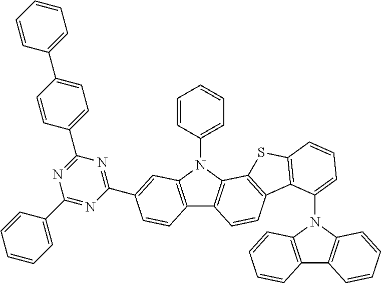 | 89% |
| 1-275 | 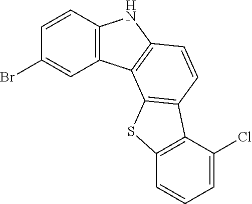 | 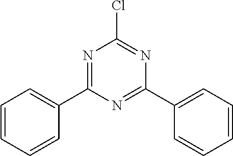 | 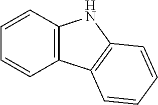 | 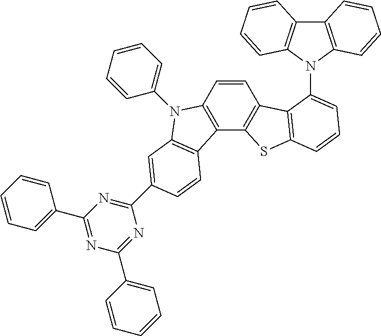 | 77% |
| 1-279 | 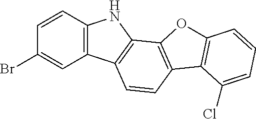 | 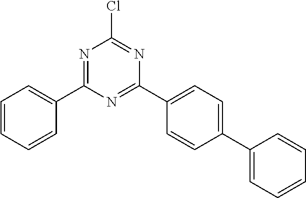 | 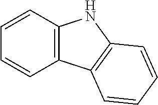 | 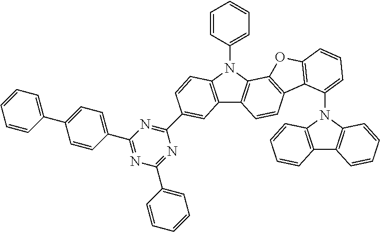 | 56% |
| 1-282 | 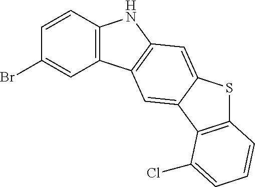 | 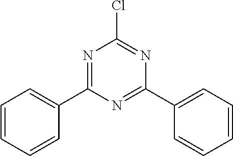 | 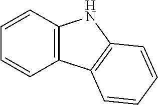 | 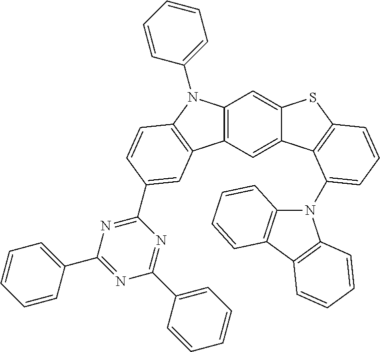 | 47% |
| 1-287 | 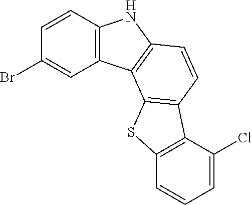 | 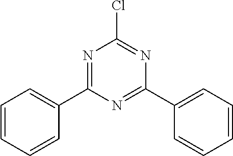 | 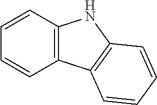 | 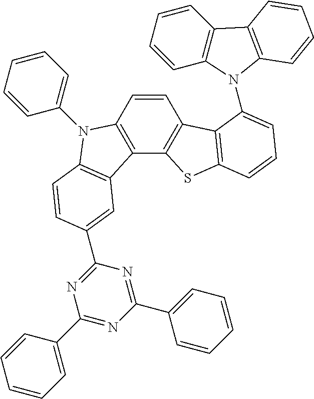 | 63% |
| 1-290 | 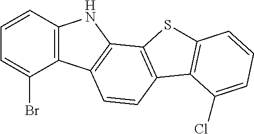 | 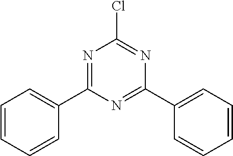 | 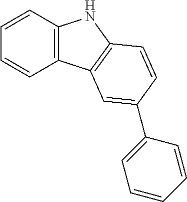 | 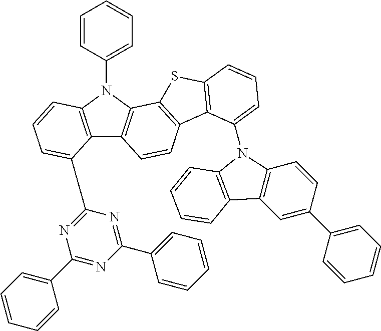 | 79% |
| 1-294 | 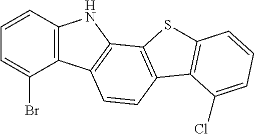 | 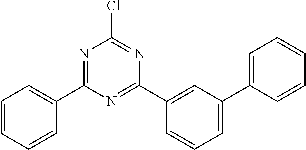 | 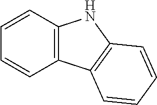 | 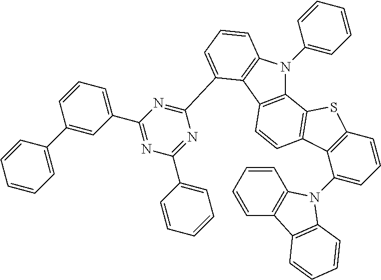 | 58% |
| 1-298 | 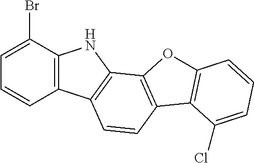 | 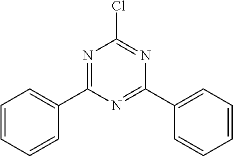 | 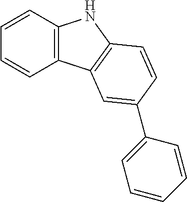 | 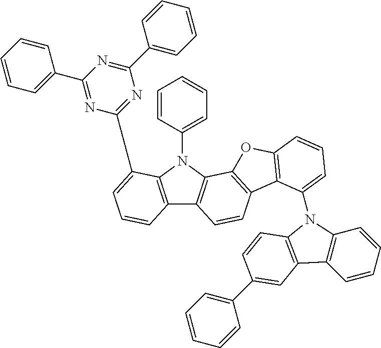 | 88% |
| 1-302 | 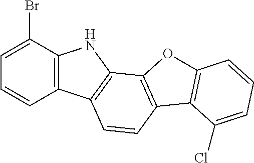 | 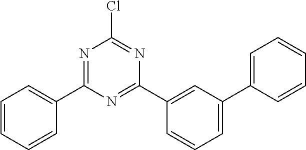 | 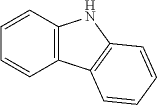 | 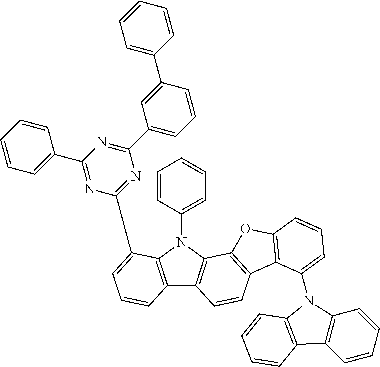 | 82% |
| 1-309 | 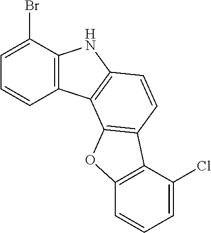 | 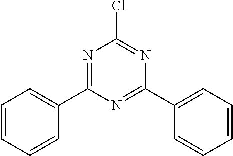 | 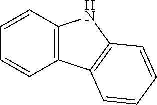 | 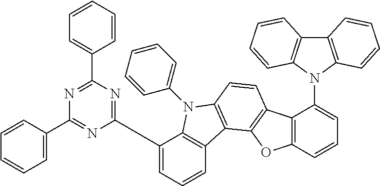 | 63% |
| 1-310 | 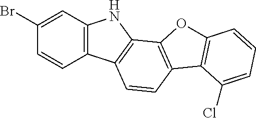 | 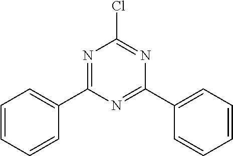 | 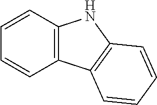 | 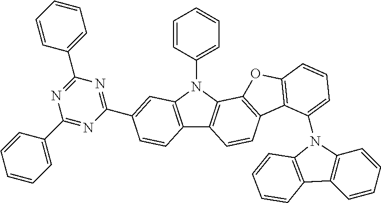 | 78% |
| 1-313 | 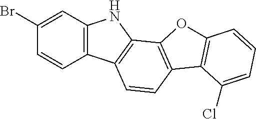 | 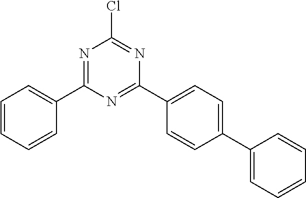 | 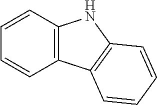 | 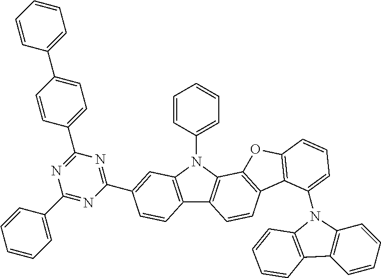 | 82% |
| 1-323 | 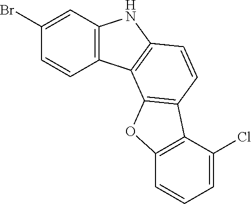 | 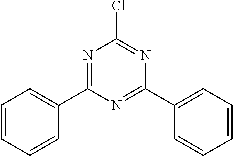 | 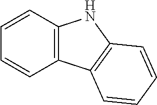 | 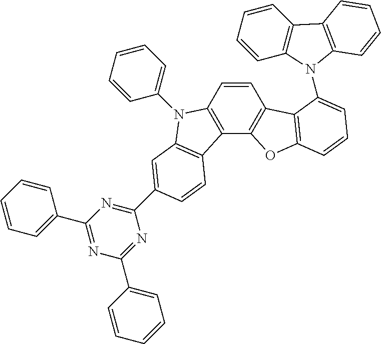 | 93% |
| 1-324 | 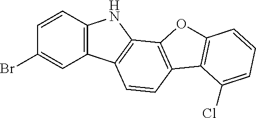 | 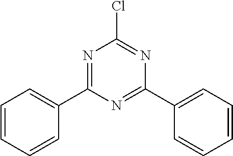 | 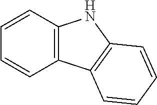 | 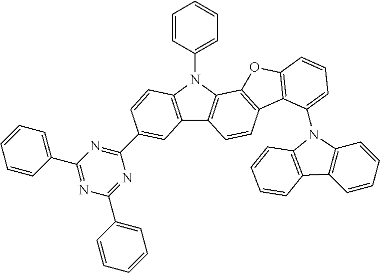 | 85% |
| 1-327 | 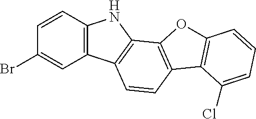 | 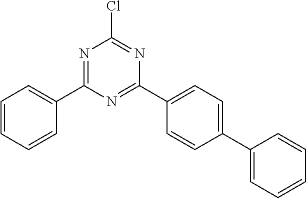 | 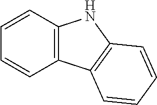 | 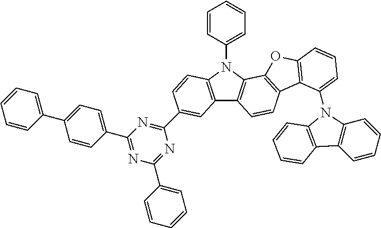 | 86% |
| 1-330 | 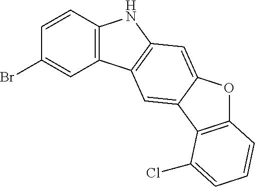 | 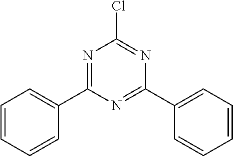 | 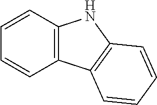 | 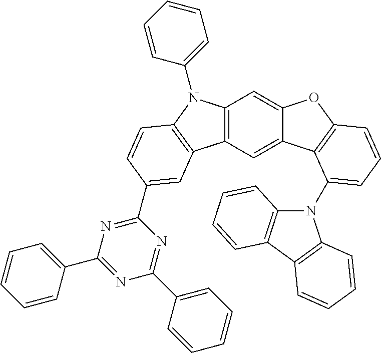 | 49% |
| 1-335 | 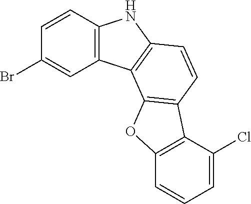 | 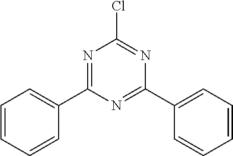 | 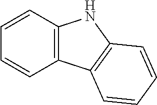 | 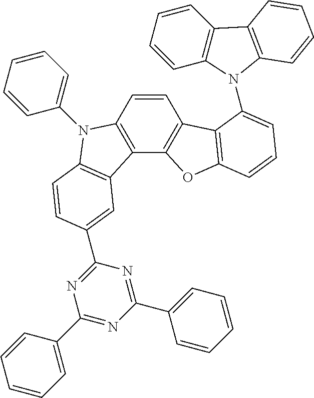 | 53% |
| 1-339 | 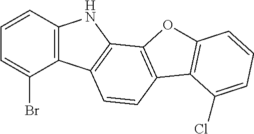 | 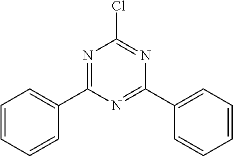 | 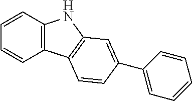 | 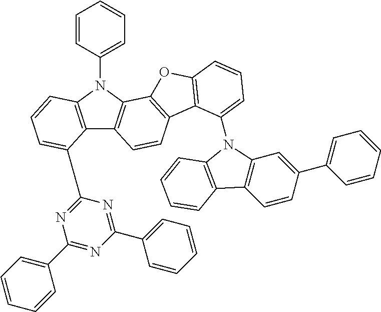 | 63% |
| 1-350 | 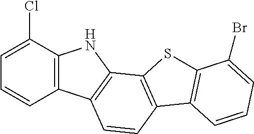 | 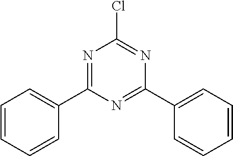 | 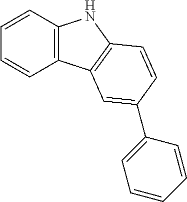 | 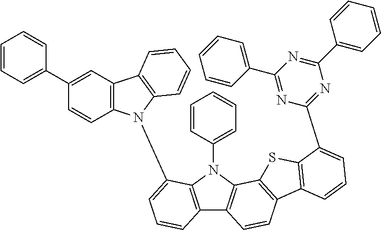 | 74% |
| 1-358 | 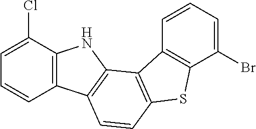 | 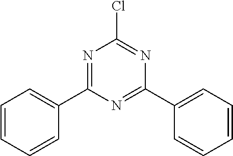 | 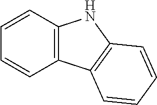 | 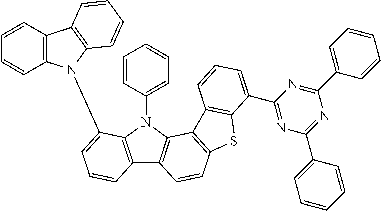 | 77% |
| 1-360 | 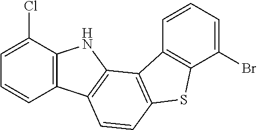 | 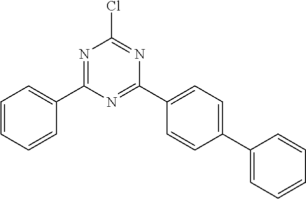 | 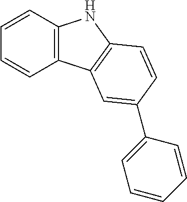 | 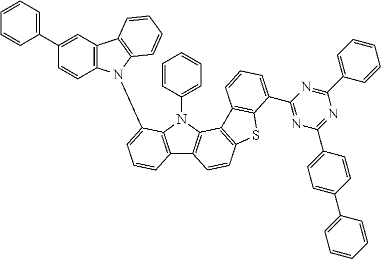 | 69% |
| 1-366 | 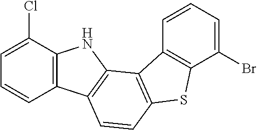 | 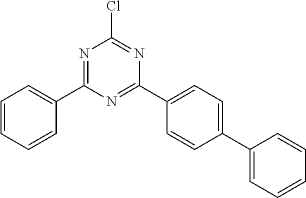 | 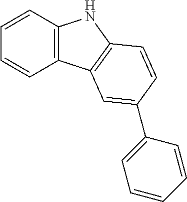 | 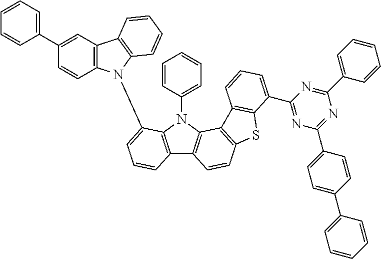 | 52% |
| 1-369 | 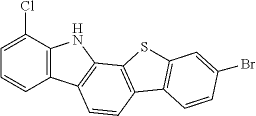 | 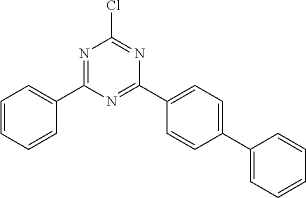 | 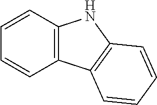 | 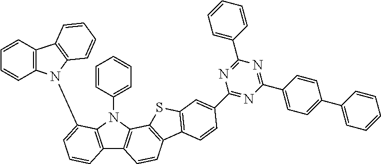 | 49% |
| 1-375 | 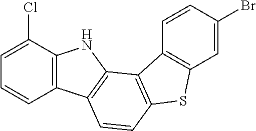 | 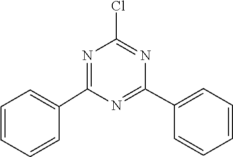 | 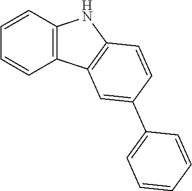 | 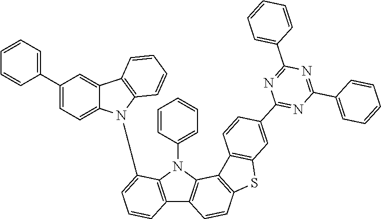 | 53% |
| 1-383 | 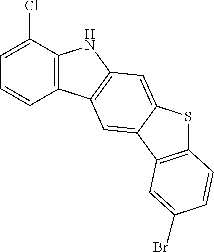 | 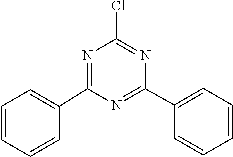 | 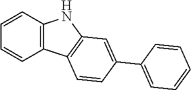 | 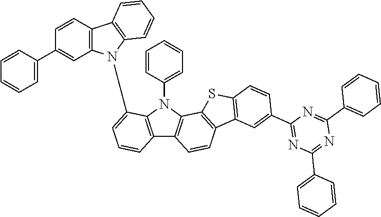 | 69% |
| 1-388 | 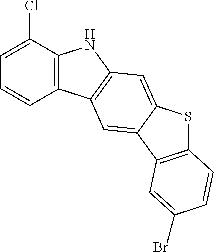 | 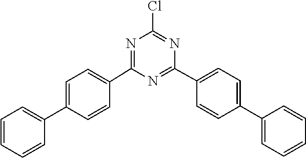 | 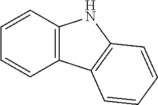 | 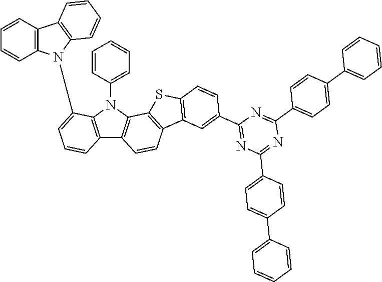 | 78% |
| 1-389 | 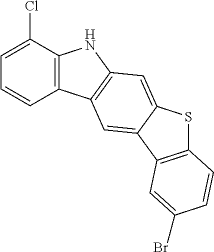 | 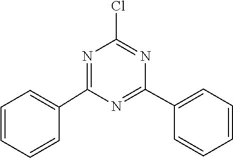 | 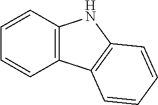 | 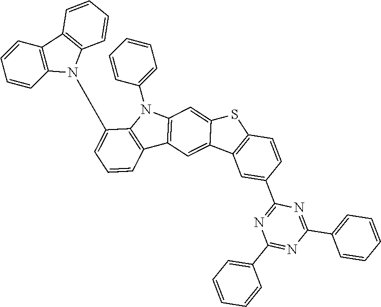 | 77% |
| 1-392 | 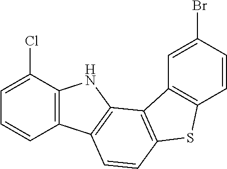 | 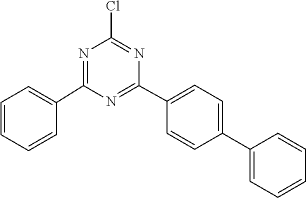 | 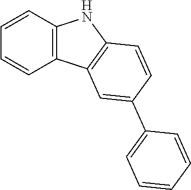 | 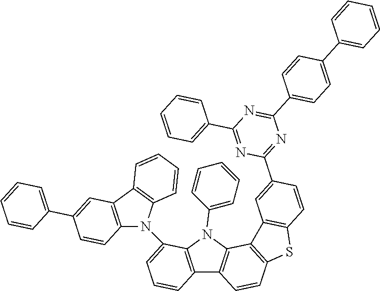 | 68% |
| 1-397 | 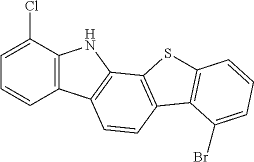 | 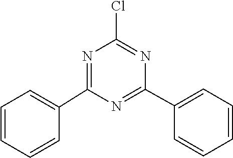 | 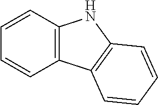 | 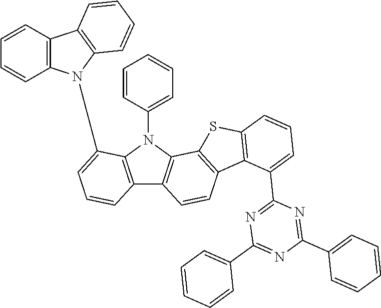 | 74% |
| 1-399 | 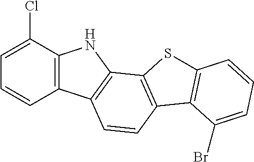 | 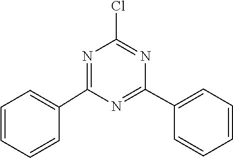 | 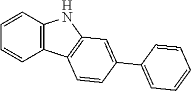 | 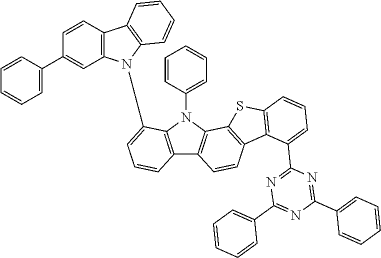 | 59% |
| 1-404 | 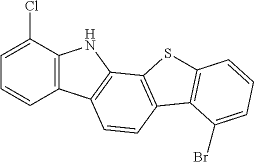 | 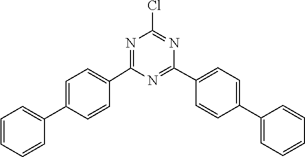 | 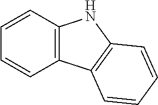 | 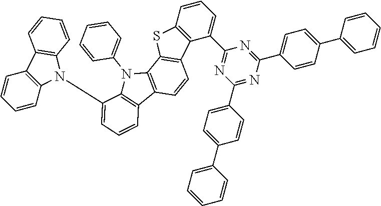 | 62% |
| 1-410 | 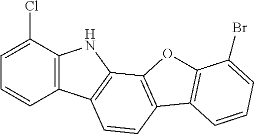 | 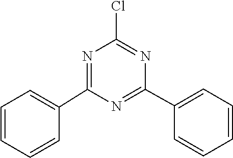 | 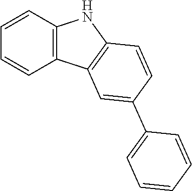 | 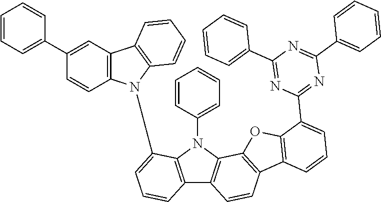 | 67% |
| 1-413 | 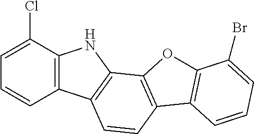 | 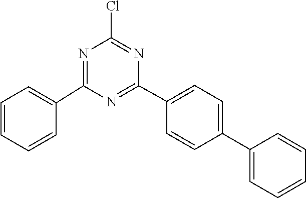 | 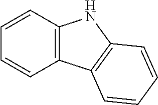 | 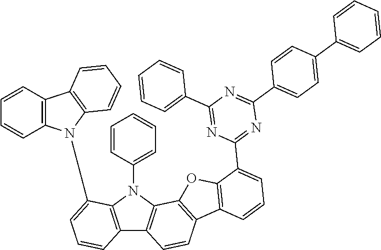 | 83% |
| 1-418 | 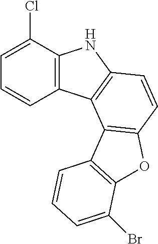 | 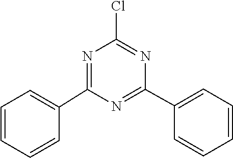 | 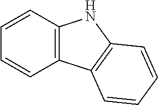 | 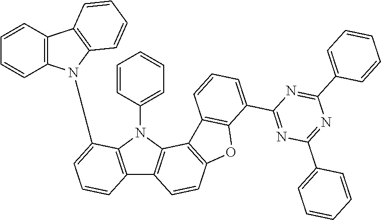 | 89% |
| 1-421 | 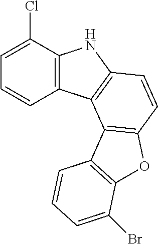 | 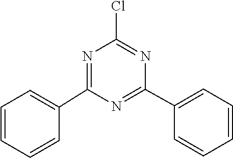 | 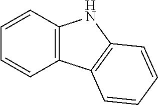 | 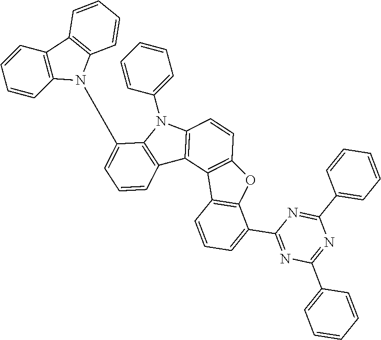 | 76% |
| 1-427 | 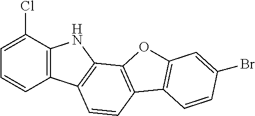 | 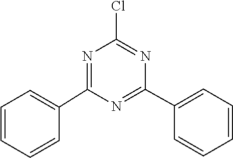 | 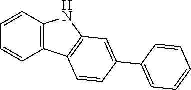 | 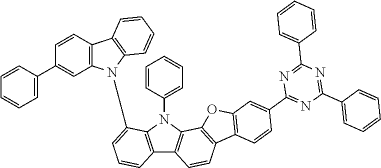 | 83% |
| 1-430 | 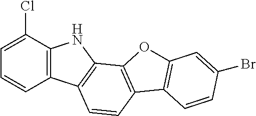 | 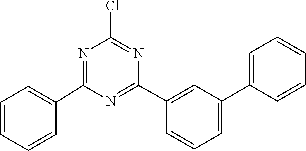 | 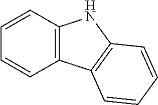 | 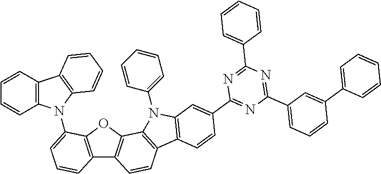 | 89% |
| 1-436 | 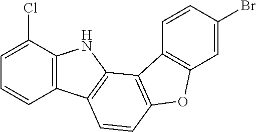 | 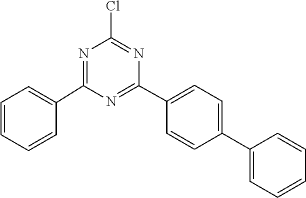 | 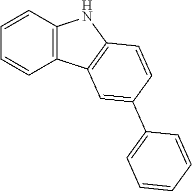 | 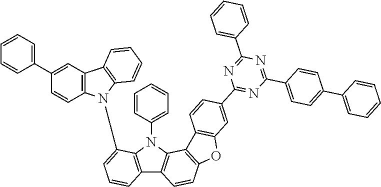 | 92% |
| 1-441 | 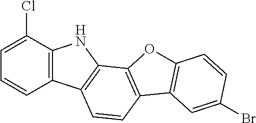 | 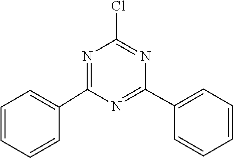 | 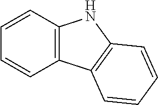 | 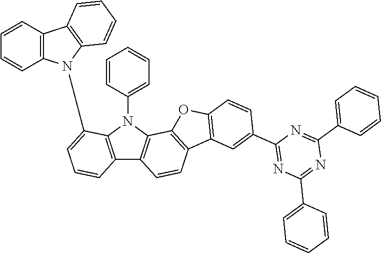 | 76% |
| 1-445 | 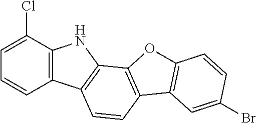 | 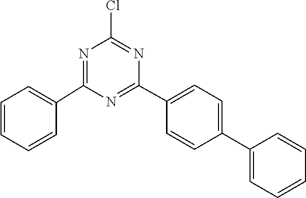 | 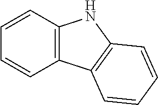 | 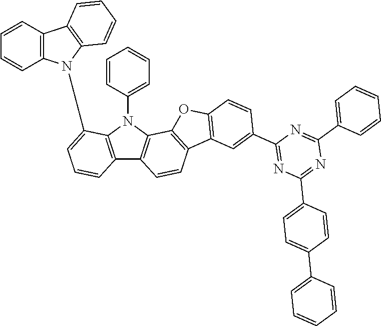 | 79% |
| 1-450 | 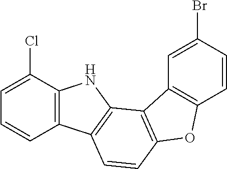 | 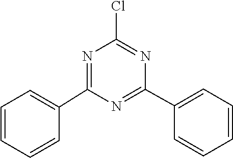 | 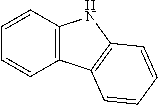 | 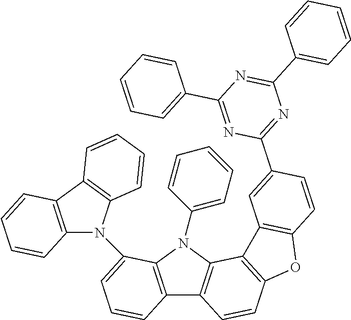 | 84% |
| 1-452 | 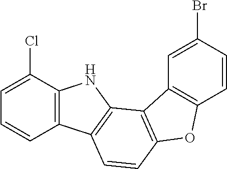 | 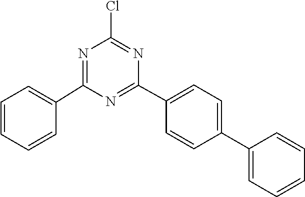 | 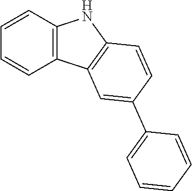 | 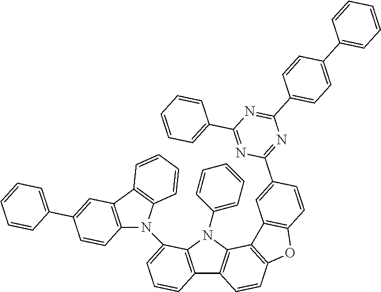 | 88% |

### <Preparation Example 2> Preparation of Compound 2-3

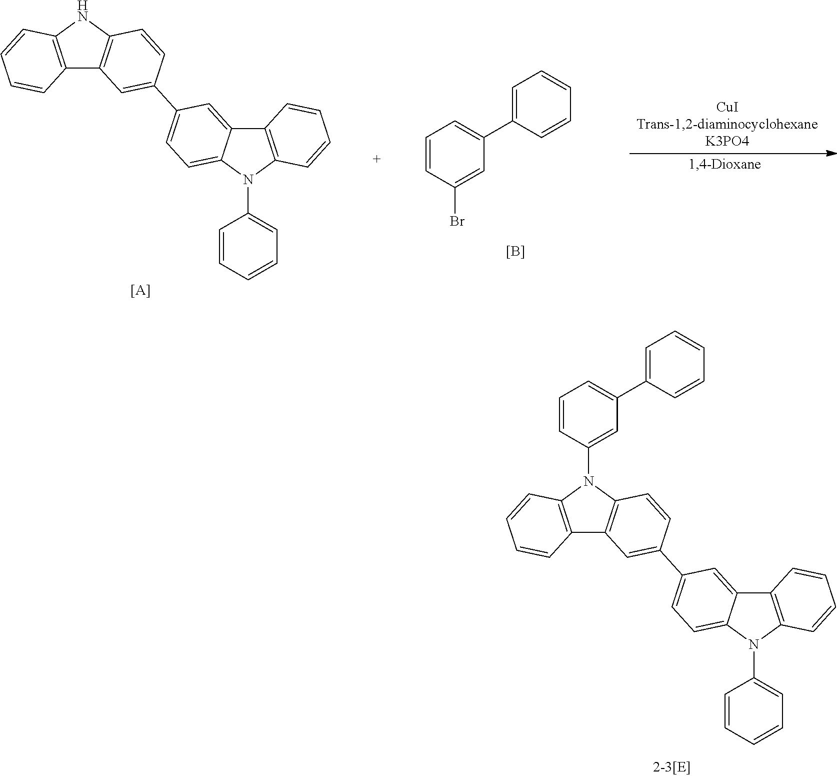

After 9-phenyl-9H,9′H-3,3′-bicarbazole (10 g, 0.24 mol), CuI (4.57 g, 0.024 mol), trans-1,4-diaminocyclohexane (2.74 g, 0.024 mol), and K₃PO₄ (10.19 g, 0.048 mol) were dissolved in 100 mL of 1,4-oxane in a one-neck round bottom flask, the resulting solution was refluxed at 125° C. for 8 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:Hex=1:3) and recrystallized with methanol to obtain Compound 2-3 [E]. (12.51 g, 93%)

Target compounds were prepared by performing preparation in the same manner as in Preparation Example 2, except that Compounds A and B of the following Table 2 were used instead of Compounds A and B in Preparation Example 2.

TABLE 2

| Compound | A | B | E | Yield |
|---|---|---|---|---|
| 2-3 | 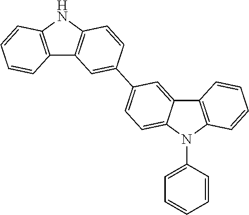 | 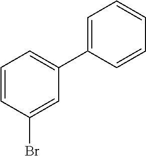 | 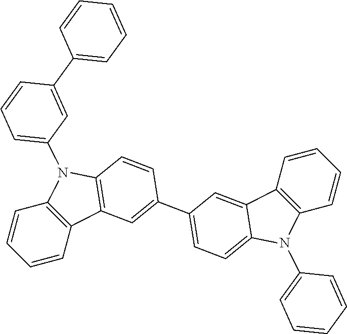 | 93% |
| 2-31 | 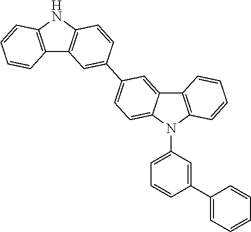 | 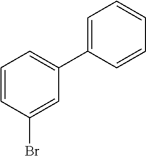 | 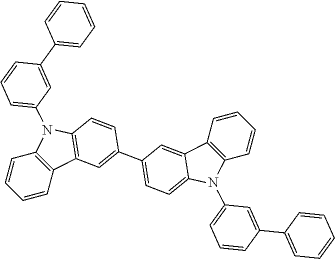 | 91% |
| 2-32 | 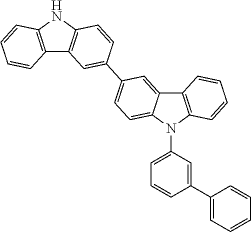 | 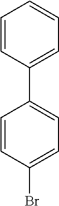 | 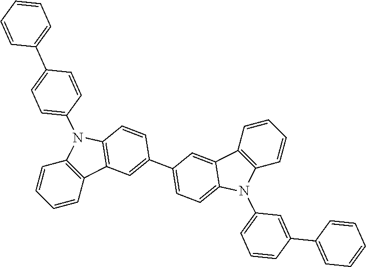 | 87% |
| 2-44 | 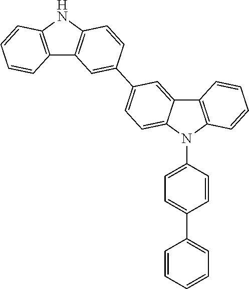 | 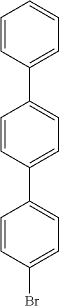 | 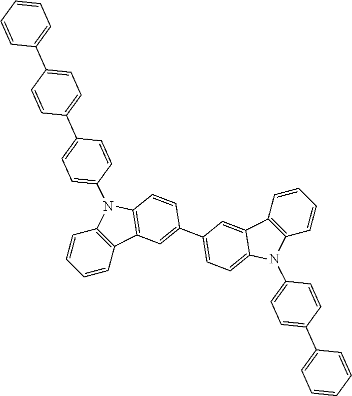 | 79% |

### <Preparation Example 3> Preparation of Compound 1-121[G]

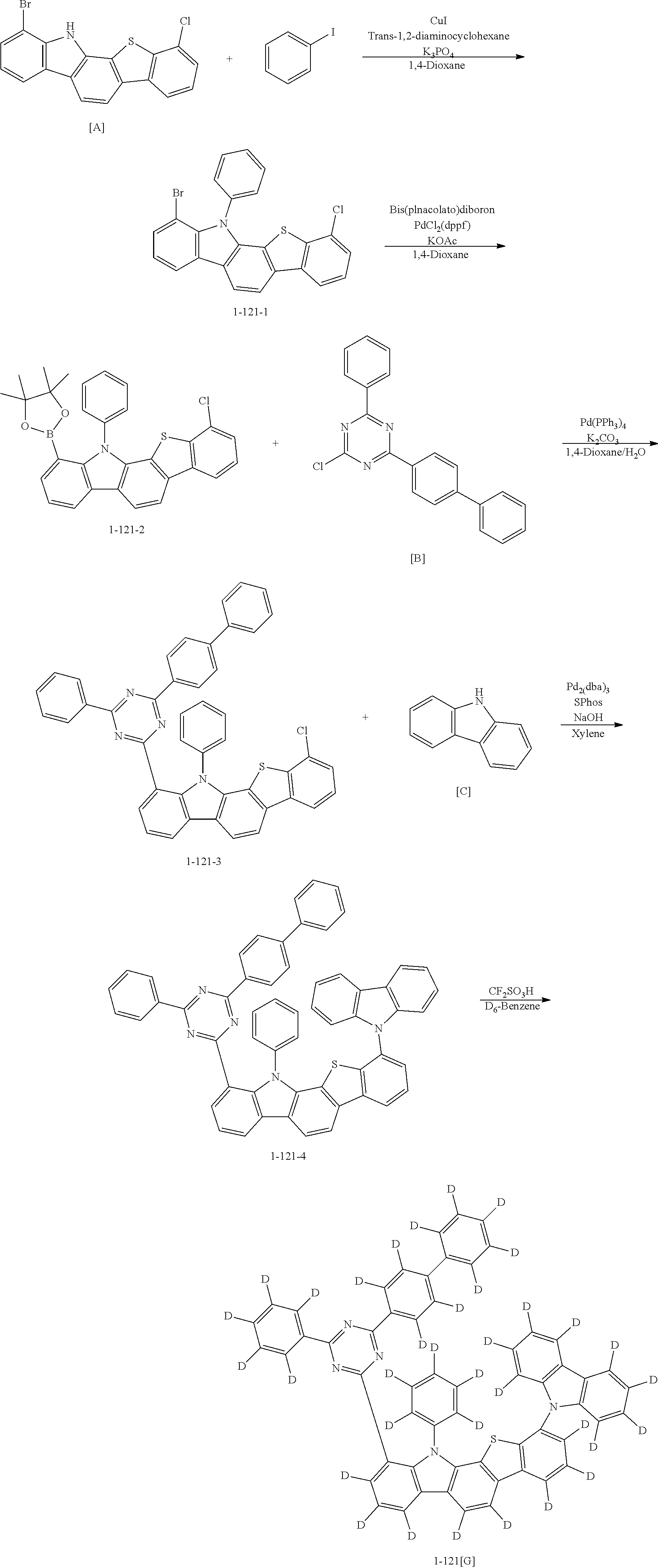

### 1) Preparation of Intermediate 1-121-1

A mixture of 1-bromo-10-chloro-12H-benzo[4,5]thieno[2,3-a]carbazole [A] (10 g, 0.026 mol), iodobenzene (6.33 g, 0.031 mol), CuI (4.95 g, 0.026 mol), trans-1,2-diaminocyclohexane (4.95 g, 0.026 mol), K₃PO₄ (11.04 g, 0.052 mol) and 1,4-dioxane (100 mL) was put into an one-neck round bottom flask and refluxed at 125° C. After the resulting product was extracted with DCM, concentrated, and then filtered with silica gel, the filtered product was concentrated, and then treated with methanol to obtain target Compound 1-121-1. (11.07 g, yield 92%)

### 2) Preparation of Intermediate 1-121-2

A mixture of 1-bromo-10-chloro-12-phenyl-12H-benzo[4,5]thieno[2,3-a]carbazole (11.07 g, 0.024 mol), bis(pinacolato)diboron (12.19 g, 0.048 mol), Pd₂(dba)₃ (2.20 g, 0.0024 mol), Sphos (1.97 g, 0.0048 mol), potassium acetate (4.71 g, 0.048 mol) and 1,4-dioxane (110 mL) was refluxed at 120° C. in a one-neck round bottom flask. The resulting product was cooled, and then concentrated and filtered with silica gel to obtain Compound 1-121-2. (11.99 g, yield 98%)

### 3) Preparation of Intermediate 1-121-3

A mixture of 10-chloro-12-phenyl-1-(4,4,5,5-tetramethyl-1,3,2-dioxaborolan-2-yl)-12H-benzo[4,5]thieno[2,3-a]carbazole (11.99 g, 0.024 mol), 2-([1,1′-biphenyl]-4-yl)-4-chloro-6-phenyl-1,3,5-triazine (9.08 g, 0.026 mol), Pd(PPh₃)₄ (1.39 g, 0.0012 mol), K₂CO₃ (6.63 g, 0.048 mol) and 1,4-dioxane (100 mL)/water (30 mL) was refluxed at 120° C. in a one-neck round bottom flask. The resulting product was cooled, and then concentrated and filtered with silica gel to obtain Compound 1-121-3. (14.43 g, yield 87%)

### 4) Preparation of Compound 1-121-4

A mixture of 1-(4-([1,1′-biphenyl]-4-yl)-6-phenyl-1,3,5-triazin-2-yl)-10-chloro-12-phenyl-12H-benzo[4,5]thieno[2,3-a]carbazole (14.43 g, 0.021 mol), 9H-carbazole (3.86 g, 0.023 mol), Pd₂(dba)₃ (1.92 g, 0.0021 mol), Sphos (1.97 g, 0.0048 mol), NaOH (1.68 g, 0.042 mol) and 1,4-dioxane (140 mL) was refluxed at 180° C. in a one-neck round bottom flask. The resulting product was cooled, and then concentrated and filtered with silica gel to obtain Compound 1-121-4. (14.67 g, yield 85%)

### 5) Preparation of Compound 1-121[G]

A mixture of 1-(4-([1,1′-biphenyl]-4-yl)-6-phenyl-1,3,5-triazin-2-yl)-10-(9H-carbazol-9-yl)-12-phenyl-12H-benzo[4,5]thieno[2,3-a]carbazole (14.67 g, 0.018 mol), triflic acid (40.8 g, 0.27 mol) and D6-benzene (140 mL) was refluxed at 70° C. in a one-neck round bottom flask. The resulting product was quenched and extracted with dichloromethane and H₂O and concentrated, and then filtered with silica gel. The filtered product was concentrated, and then treated with methanol to obtain Compound 1-121[G]. (12.81 g, 83%)

Target compounds were prepared by performing preparation in the same manner as in Preparation Example 3, except that Compounds A to C of the following Table 3 were used instead of Compounds A to C in Preparation Example 3.

TABLE 3

| Compound | A | B | C | G | Yield |
|---|---|---|---|---|---|
| 1-121 | 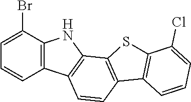 | 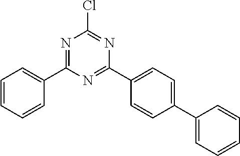 | 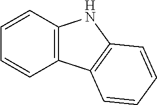 | 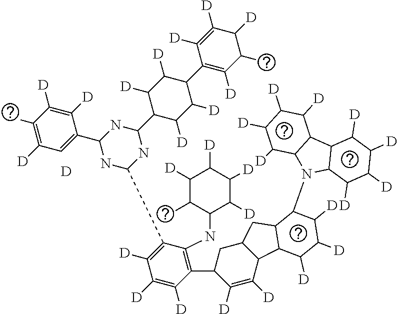 | 83% |
| 1-247 | 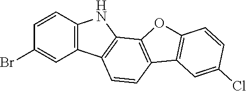 | 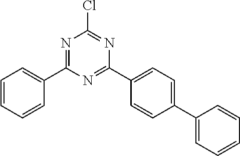 | 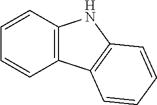 | 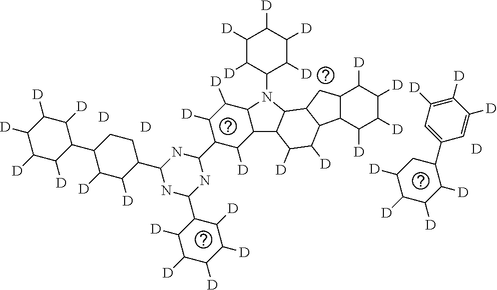 | 64% |
| 1-348 | 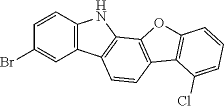 | 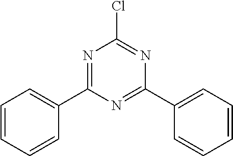 | 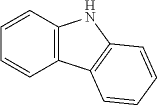 | 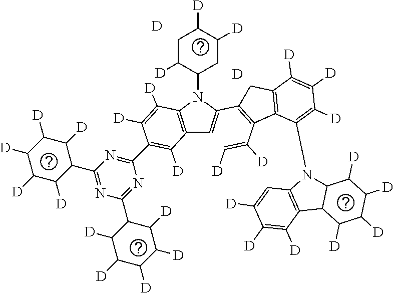 | 76% |
| 1-469 | 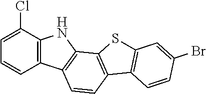 | 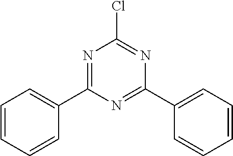 | 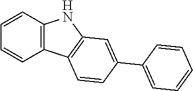 | 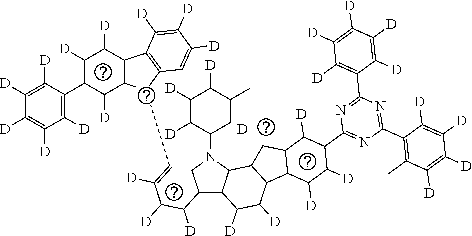 | 68% |

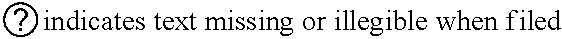

### <Preparation Example 4> Preparation of Compound 2-146 [F]

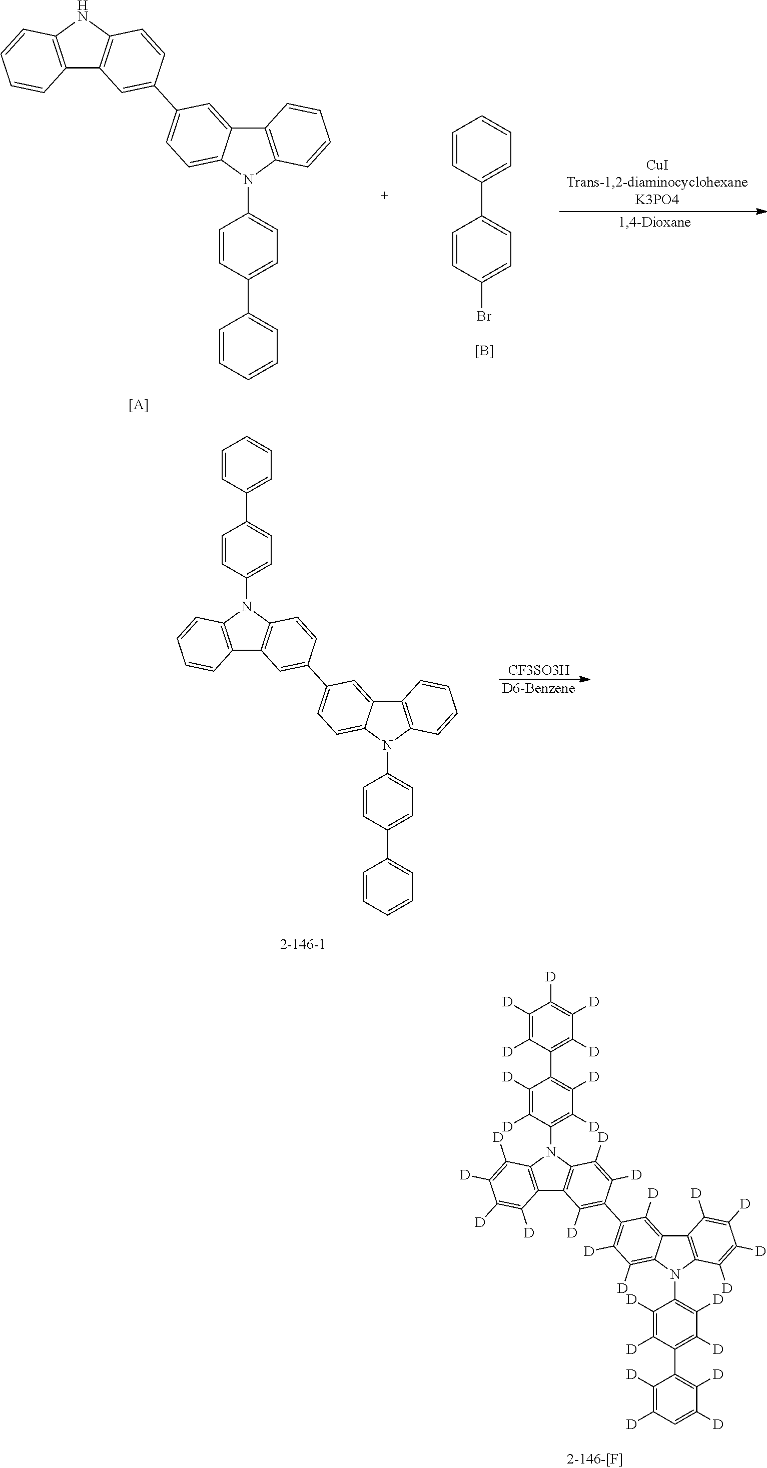

### 1) Preparation of Intermediate 2-146-1

After 9-([1,1′-biphenyl]-4-yl)-9H,9′H-3,3′-bicarbazole (10 g, 0.021 mol), CuI (0.40 g, 0.0021 mol), trans-1,4-diaminocyclohexane (0.024 g, 0.0021 mol), and K₃PO₄ (8.92 g, 0.042 mol) were dissolved in 100 mL of 1,4-oxane in a one-neck round bottom flask, the resulting solution was refluxed at 125° C. for 8 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:Hex=1:3) and recrystallized with methanol to obtain Compound 2-146-1. (12.17 g, 91%)

### 2) Preparation of Compound 2-146 [F]

A mixture of 9,9′-di([1,1′-biphenyl]-4-yl)-9H,9′H-3,3′-bicarbazole (12.17 g, 0.017 mol), triflic acid (40.8 g, 0.27 mol) and D6-benzene (120 mL) was refluxed at 70° C. in a one-neck round bottom flask. The resulting product was quenched and extracted with dichloromethane and H₂O and concentrated, and then filtered with silica gel. The filtered product was concentrated, and then treated with methanol to obtain Compound 2-146[F]. (8.87 g, 78%)

Target compounds were prepared by performing preparation in the same manner as in Preparation Example 4, except that Compounds A and B of the following Table 4 were used instead of Compounds A and B in Preparation Example 4.

TABLE 4

| Compound | A | B | F | Yield |
|---|---|---|---|---|
| 2-146 | 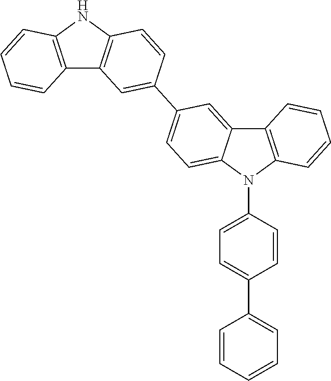 | 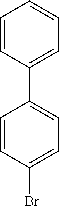 | 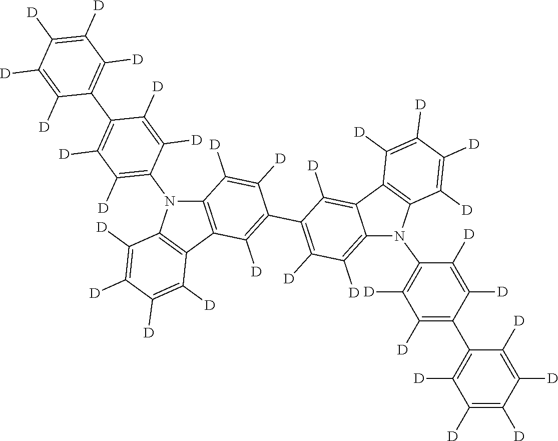 | 78% |
| 2-149 | 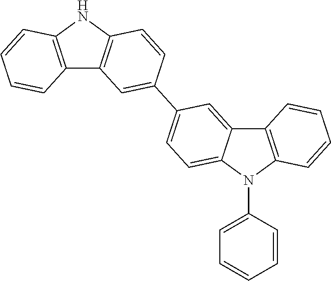 | 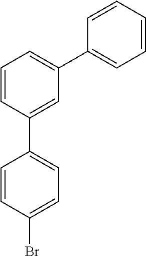 | 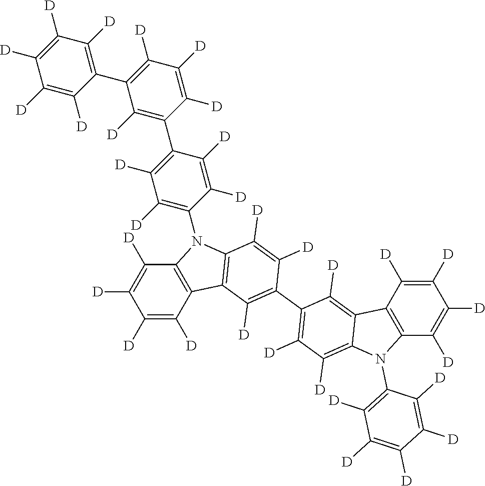 | 91% |
| 2-154 | 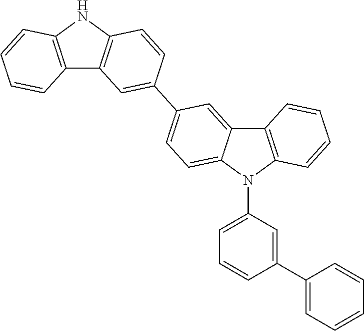 | 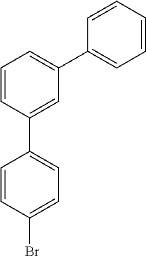 | 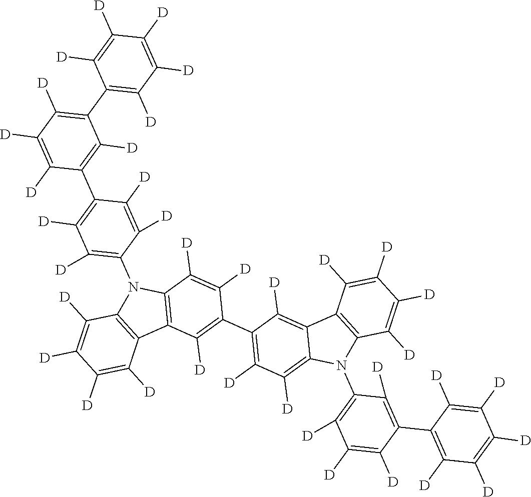 | 93% |
| 2-158 | 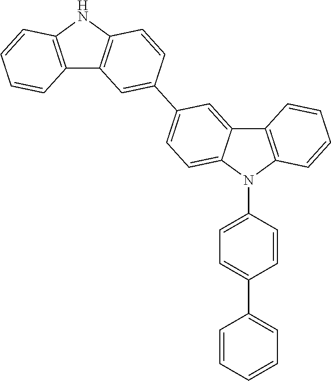 | 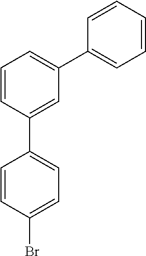 | 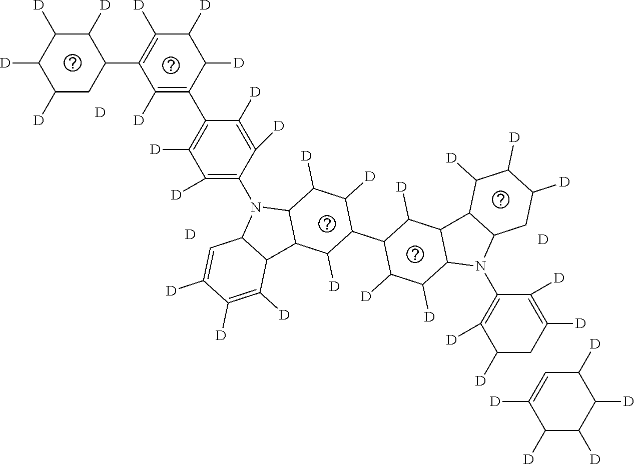 | 76% |

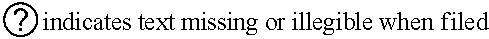

### <Preparation Example 5> Preparation of Compound 2-98[I]

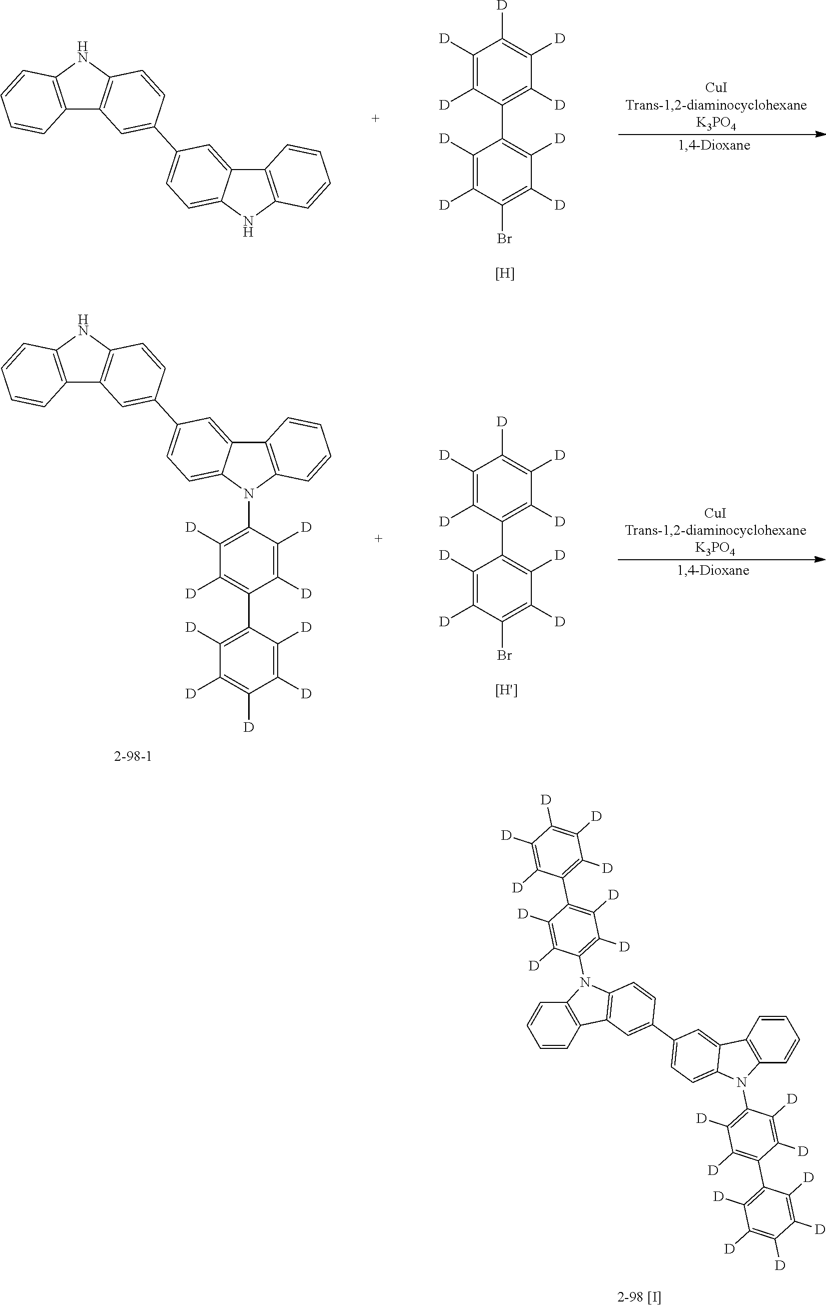

### 1) Preparation of Intermediate 2-98-1

After 9H,9′H-3,3′-bicarbazole (10 g, 0.030 mol), 4-bromo-1,1′-biphenyl-2,2′,3,3′,4′,5,5′,6,6′-D9 [H] (7.26 g, 0.030 mol), CuI (0.57 g, 0.003 mol), trans-1,2-diaminocyclohexane (0.34 g, 0.003 mol), and K₃PO₄ (12.74 g, 0.06 mol) were dissolved in 100 mL of 1,4-dioxane in a one-neck round bottom flask, the resulting solution was refluxed at 125° C. for 8 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:hexane=1:3) and recrystallized with methanol to obtain Intermediate 2-98-1. (13.92 g, yield 94%)

### 2) Preparation of Compound 2-98[I]

After Intermediate 2-2-1 (13.92 g, 0.028 mol), 4-bromo-1,1′-biphenyl-2,2′,3,3′,4′,5,5′,6,6′-D9 [H′] (6.83 g, 0.028 mol), CuI (0.53 g, 0.0028 mol), trans-1,2-diaminocyclohexane (0.32 g, 0.0028 mol), and K₃PO₄ (11.89 g, 0.056 mol) were dissolved in 140 mL of 1,4-dioxane in a one-neck round bottom flask, the resulting solution was refluxed at 125° C. for 8 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:hexane=1:3) and recrystallized with methanol to obtain target Compound 2-98. (16.14 g, yield 88%)

When Compound H and Compound H′ are the same, the target compound may be immediately synthesized by adding 2 equivalents of Compound H in Preparation Example 5-1. That is, when Compound H and Compound H′ are the same, the aforementioned Preparation Example 5-2 may be omitted.

Synthesis was performed in the same manner as in Preparation Example 5, except that Compounds H and H′ of the following Table 5 were used in Preparation Example 5.

TABLE 5

| Compound | H | H′ | I | Yield |
|---|---|---|---|---|
| 2-98 | 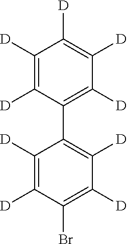 | 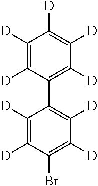 | 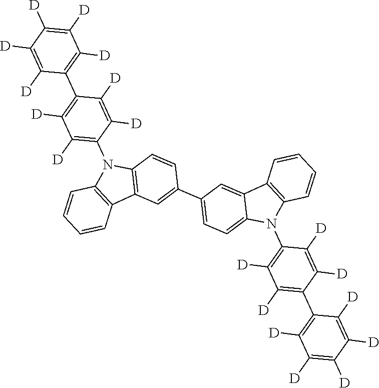 | 88% |
| 2-101 | 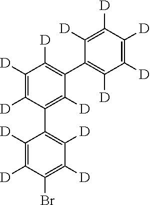 | 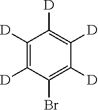 | 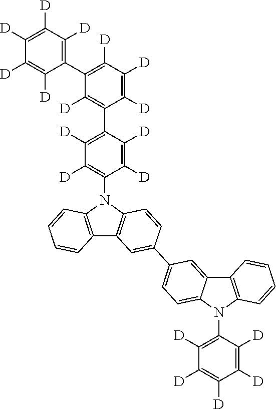 | 77% |
| 2-110 | 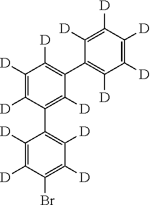 | 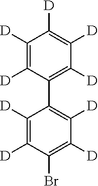 | 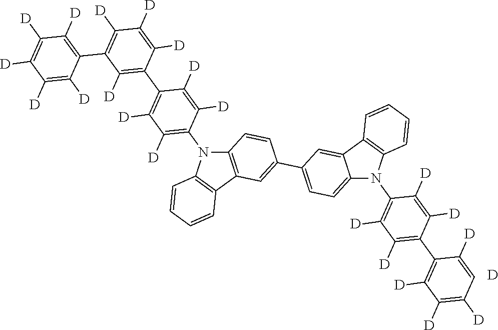 | 71% |

### <Preparation Example 6> Preparation of Compound 2-122 [J]

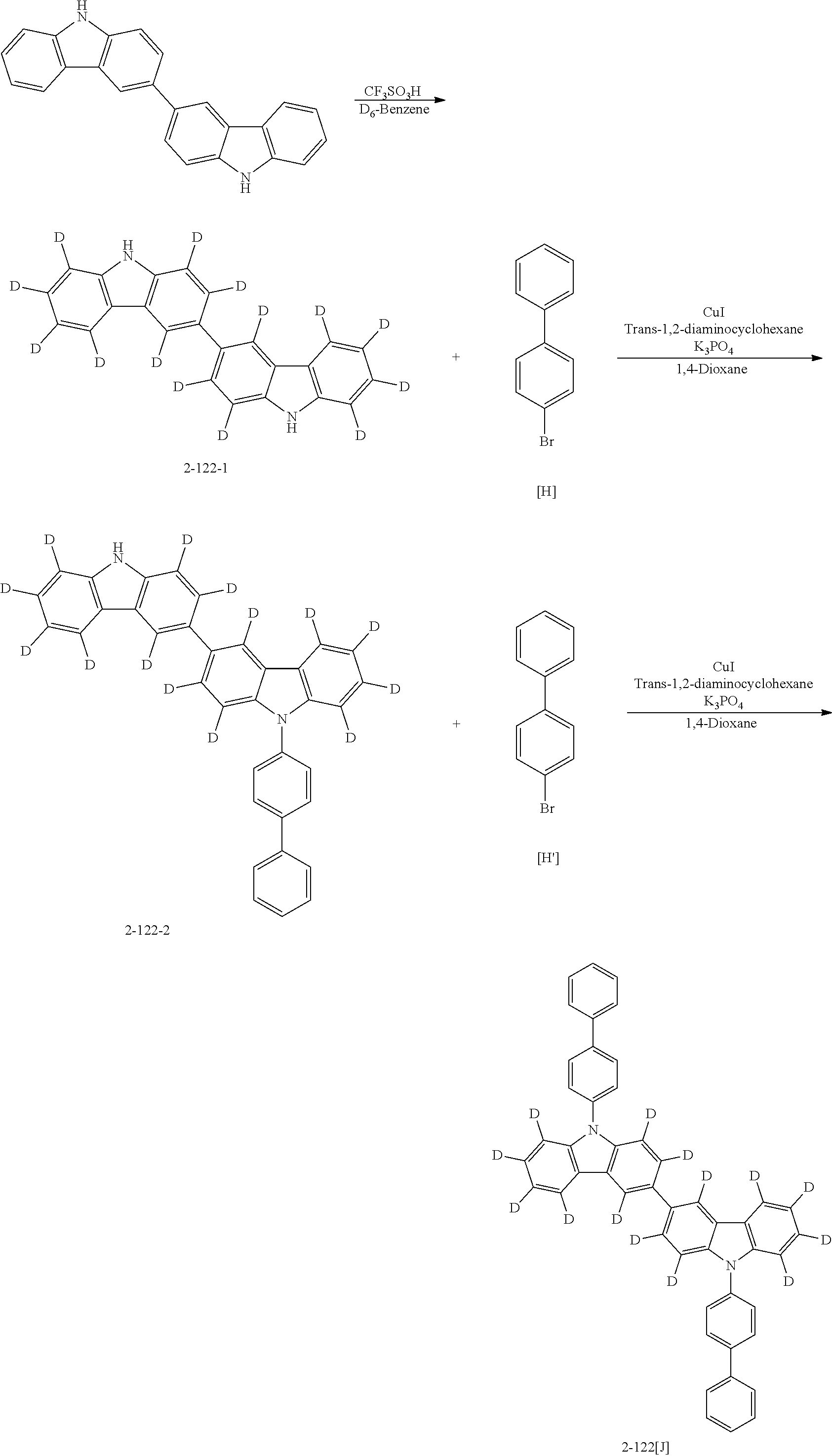

### 1) Preparation of Intermediate 2-122-1

A mixture of 9H,9′H-3,3′-bicarbazole (10 g, 0.030 mol), triflic acid (112.56 g, 0.75 mol) and D₆-benzene (500 mL) was refluxed at 40° C. in a one-neck round bottom flask. The resulting product was quenched and extracted with DCM and H₂O and concentrated, and then filtered with silica gel. The filtered product was concentrated, and then treated with methanol to obtain Intermediate 2-122-1. (7.07 g, yield 68%)

### 2) Preparation of Intermediate 2-122-2

After Intermediate 2-122-1 (7.07 g, 0.02 mol), CuI (0.38 g, 0.002 mol), 4-bromo-1,1′-biphenyl [H] (4.66 g, 0.02 mol, trans-1,2-diaminocyclohexane (0.23 g, 0.002 mol), and K₃PO₄ (8.49 g, 0.04 mol) were dissolved in 70 mL of 1,4-dioxane in a one-neck round bottom flask, the resulting solution was refluxed at 125° C. for 8 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:hexane=1:3) and recrystallized with methanol to obtain Intermediate 2-122-2. (8.28 g, yield 83%)

### 3) Preparation of Compound 2-122[J]

After Intermediate 2-26-2 (8.28 g, 0.017 mol), CuI (0.32 g, 0.0017 mol), 4-bromo-1,1′-biphenyl [H′] (3.96 g, 0.017 mol), trans-1,2-diaminocyclohexane (0.19 g, 0.0017 mol), and K₃PO₄ (7.22 g, 0.034 mol) were dissolved in 80 mL of 1,4-dioxane in a one-neck round bottom flask, the resulting solution was refluxed at 125° C. for 8 hours. After the reaction was completed, distilled water and DCM were added thereto at room temperature, extraction was performed, the organic layer was dried over MgSO₄, and then the solvent was removed using a rotary evaporator. The reaction product was purified by column chromatography (DCM:hexane=1:3) and recrystallized with methanol to obtain target Compound 2-122. (8.63 g, yield 78%)

When Compound H and Compound H′ are the same, the target compound may be immediately synthesized by adding 2 equivalents of Compound H in Preparation Example 6-2. That is, when Compound H and Compound H′ are the same, the aforementioned Preparation Example 6-3 may be omitted.

Synthesis was performed in the same manner as in Preparation Example 6, except that Compounds H and H′ of the following Table 6 were used in Preparation Example 6.

TABLE 6

| Compound | H | H′ | J | Yield |
|---|---|---|---|---|
| 2-122 | 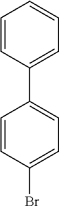 | 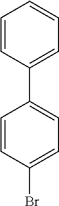 | 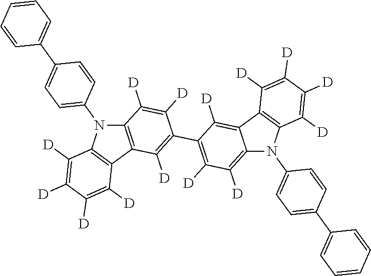 | 78% |
| 2-130 | 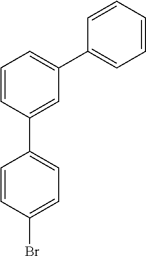 | 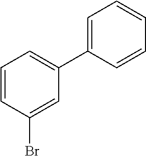 | 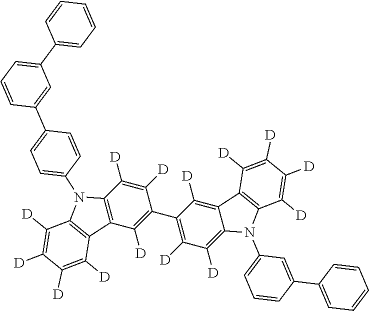 | 83% |

The other heterocyclic compound of Chemical Formula 1 or 2 other than the compounds described in Preparation Examples 1 to 6 and Tables 1 to 6 was also prepared in the same manner as in the above-described Preparation Examples, and synthesis results are shown in the following Tables 7 and 8. The following Table 7 shows the measured values of ¹H NMR (DMSO, 200 Mz), and the following Table 8 shows the measured values of field desorption mass spectrometry (FD-MS).

TABLE 7

| Compound | ¹H NMR (DMSO, 300 Mz) |
|---|---|
| 1-1 | δ = 8.65 (1H, d), 8.55 (1H, d), 7.58~8.41 (6H, m), 8.19 |
| | (1H, d), 8.05 (1H, d), 7.94 (1H, d), 7.35~7.65 (18H, |
| | m), 7.16~7.20 (2H, m) |
| 1-5 | δ = 8.65 (1H, d), 8.55 (1H, d), 8.30~8.41 (4H, m), 8.19 |
| | (1H, d), 8.05 (1H, d), 7.94~7.96 (3H, m), 7.75 (2H, d), |
| | 7.35~7.65 (18H, m), 7.16~7.25 (4H, m) |
| 1-12 | δ = 8.65 (1H, d), 8.55 (1H, d), 8.30~8.41 (4H, m), 8.08 |
| | (1H, d), 7.89~7.99 (5H, m), 7.75~7.80 (6H, m), |
| | 7.35~7.62 (18H, m), 7.25 (2H, d), 7.16 (1H, t) |
| 1-22 | δ = 8.55~8.62 (2H, m), 8.36~8.41 (4H, m), 8.19~8.22 |
| | (2H, m), 8.05 (1H, d), 7.94 (2H, m), 7.35~7.75 (20H, |
| | m), 7.16~7.20 (2H, t) |
| 1-32 | δ = 8.62 (1H, d), 8.55 (1H, d), 8.22~8.41 (7H, m), |
| | 8.05~8.06 (2H, d), 7.94 (1H, d), 7.74 (1H, s), |
| | 7.33~7.65 (17H, m), 7.16~7.19 (5H, m) |
| 1-38 | δ = 8.55 (1H, d), 8.30~8.41 (5H, m), 8.13~8.19 (2H, m), |
| | 8.05 (1H, d), 7.89~7.94 (3H, m), 7.35~7.75 (21H, m), |
| | 7.16~7.20 (2H, t) |
| 1-42 | δ = 8.55 (1H, d), 7.50~8.36 (6H, m), 8.08~8.19 (3H, m), |
| | 7.89~7.94 (2H, m), 7.80 (1H, d), 7.35~7.65 (16H, m), |
| | 7.16~7.20 (2H, t) |
| 1-46 | δ = 8.55 (1H, d), 8.30~8.41 (7H, m), 8.13 (1H, d), |
| | 7.89~7.96 (4H, m), 7.74~7.75 (3H, m), 7.41~7.65 (18H, |
| | m), 7.16 (1H, t) |
| 1-50 | δ = 8.55 (1H, d), 8.36~8.41 (5H, m), 8.22 (1H, d), |
| | 7.89~8.05 (5H, m), 7.75~7.77 (3H, m), 7.35~7.62 (19H, |
| | m), 7.16 (1H, t) |
| 1-53 | δ = 8.55 (1H, d), 8.36~8.41 (3H, m), 8.19~8.22 (2H, m), |
| | 8.04~8.05 (2H, d). 7.94~7.96 (3H, m), 7.75 (2H, d), |
| | 7.35~7.65 (18H, m), 7.16~7.25 (4H, m) |
| 1-60 | δ = 8.55 (1H, d), 8.36~8.41 (3H, m), 8.22 (1H, d), |
| | 7.89~8.08 (7H, m), 7.75~7.80 (6H, m), 7.41~7.65 (18H, |
| | m), 7.25 (2H, d), 7.16 (1H, t) |
| 1-63 | δ = 8.65 (1h, d), 8.55 (1H, d), 8.30~8.58 (6H, m), |
| | 7.91~7.94 (3H, m), 7.84 (1H, d), 7.74~7.75 (3H, d), |
| | 7.31~7.62 (19H, m), 7.16 (1H, t) |
| 1-65 | δ = 8.65 (1H, d), 8.55 (1H, d), 8.30~8.36 (3H, m), 8.19 |
| | (1H, d), 7.94~7.96 (4H, m), 7.84 (1H, d), 7.75 (2H, d), |
| | 7.16~7.62 (22H, m) |
| 1-72 | δ = 8.65 (1H, d), 8.55 (1H, d), 8.30~8.36 (3H, m), |
| | 7.89~7.99 (6H, m), 7.75~7.77 (5H, m), 7.25~7.62 (22H, |
| | m), 7.16 (1H, t) |
| 1-79 | δ = 8.62 (1H, d), 8.55 (1H, d), 8.31~8.36 (3H, m), 8.22 |
| | (1H, d), 7.35~7.94 (3H, m), 7.84 (1H, d), 7.74~7.75 |
| | (3H, d), 7.31~7.62 (18H, m), |
| 1-81 | δ = 8.55 (1H, d), 8.55 (1H, d), 8.36 (2H, m), 7.19~8.22 |
| | (2H, m), 7.94~7.96 (4H, m), 7.84 (1H, d), 7.74~7.75 |
| | (3H, d), 7.20~7.62 (21H, m) |
| 1-88 | δ = 8.62 (1H, d), 8.55 (1H, d), 8.36 (2H, m), 8.22 (1H, |
| | d), 7.89~7.99 (6H, m), 7.74~7.77 (6H, m), 7.31~7.68 |
| | (21H, m), 7.16 (1H, t) |
| 1-91 | δ = 8.86 (1H, d), 8.55 (1H, d), 8.36 (4H, m), 8.19~8.22 |
| | (2H, m), 7.94 (2H, d), 7.84 (1H, d), 7.74 (1H, s), |
| | 7.46~7.62 (14H, m), 7.31~7.35 (2H, m), 7.13~7.20 (3H, |
| | m) |
| 1-97 | δ = 8.55 (1H, d), 8.30~8.36 (3H, m), 8.13~8.19 (2H, m), |
| | 7.84~7.96 (6H, m), 7.75 (2H, d), 7.16~7.62 (21H, m) |
| 1-100 | δ = 8.55 (1H, d), 8.30 (1H, d), 8.13~8.19 (2H, m), |
| | 7.84~7.96 (8H, m), 7.75 (4H, d), 7.16~7.62 (23H, m) |
| 1-102 | δ = 8.55 (1H, d), 8.30~8.36 (5H, m), 8.13~8.19 (2H, m), |
| | 7.89~7.94 (3H, m), 7.46~7.62 (16H, m), 7.31~7.35 (2H, |
| | m), 7.16~7.20 (2H, t) |
| 1-108 | δ 8.55 (1H, d), 8.29~8.36 (6H, m), 8.13 (1H, d), 8.06 |
| | (1H, d), 7.84~7.94 (4H, m), 7.31~7.62 (16H, m), |
| | 7.13~7.19 (6H, m) |
| 1-113 | δ = 8.55 (1H, d), 8.36 (2H, m), 8.19~8.22 (2H, m), 8.04 |
| | (1H, d), 7.94~7.96 (4H, m), 7.84 (1H, d), 7.75 (2H, d), |
| | 7.16~7.62 (22H, m) |
| 1-120 | δ = 8.55 (1H, d), 8.36 (2H, m), 8.22 (1H, d), 7.89~8.04 |
| | (7H, m), 7.75~7.77 (5H, m), 7.25~7.94 (22H, m), 7.16 |
| | (1H, t) |
| 1-121 | δ = deuterium content of 100% with no ¹H NMR peak |
| 1-125 | δ = 8.65 (1H, d), 8.55 (1H, d), 8.30~8.36 (5H, m), 8.19 |
| | (1H, d), 8.05~8.10 (2H, m), 7.90~7.94 (2H, m), |
| | 7.36~7.60 (17H, m), 7.16~7.20 (2H, m) |
| 1-126 | δ = 8.65 (1H, d), 8.55 (1H, d), 8.30~8.36 (5H, m), |
| | 7.89~8.10 (6H, m), 7.75~7.77 (3H, m), 7.35~7.62 (18H, |
| | m), 7.16 (1H, t) |
| 1-129 | δ = 8.65 (1H, d), 8.55 (1H, d), 8.30~8.36 (3H, m), 8.19 |
| | (1H, d), 8.05~8.10 (2H, m), 7.90~7.96 (4H, m), 7.75 |
| | (2H, d), 7.35~7.62 (17H, m), 7.16~7.25 (4H, m) |
| 1-132 | δ = 8.65 (1H, d), 8.55 (1H, d), 8.30 (1H, d), 8.19 (1H, |
| | d), 8.05~8.10 (2H, m), 7.90~7.96 (6H, m), 7.75 (4H, d), |
| | 7.35~7.62 (22H, m), 7.16~7.25 (6H, m) |
| 1-141 | δ = 8.62 (1H, d), 8.55 (1H, d), 8.36 (4H, m), 8.19~8.22 |
| | (2H, m), 8.05~8.10 (2H, m), 7.90~7.94 (2H, m), 7.74 |
| | (1H, s), 7.35~7.62 (16H, m), 7.16~7.20 (2H, m) |
| 1-143 | δ = 8.62 (1H, d), 8.55 (1H, d), 8.31~8.36 (5H, m), 8.22 |
| | (1H, d), 8.05~8.10 (2H, m), 7.90~7.94 (3H, m), |
| | 7.74~7.75 (4H, d), 7.35~7.62 (17H, m), 7.16 (1H, t) |
| 1-145 | δ = 8.55~8.62 (2H, m), 8.36 (2H, m), 8.19~8.22 (2H, m), |
| | 8.05~8.10 (2H, m), 7.90~7.96 (4H, m), 7.74~7.75 (3H, |
| | d), 7.35~7.62 (16H, m), 7.16~7.25 (4H, m) |
| 1-150 | δ = 8.62 (1H, d), 8.55 (1H, d), 8.36 (4H, m), 8.19~8.22 |
| | (2H, m), 8.08~8.10 (2H, t), 7.90~7.94 (2H, m), 7.80 |
| | (1H, d), 7.74 (1H, s), 7.43~7.62 (15H, m), 7.16~7.20 |
| | (2H, m) |
| 1-154 | δ = 8.62 (1H, d), 8.55 (1H, d), 8.31~8.36 (5H, m), 8.22 |
| | (1H, d), 8.10 (1H, d), 7.90~7.96 (4H, m), 7.74~7.75 |
| | (4H, d), 7.31~7.62 (17H, m), 7.16 (1H, t) |
| 1-158 | δ = 8.55 (1H, d), 8.30~8.36 (5H, m), 7.89~8.13 (8H, m), |
| | 7.75~7.77 (3H, m), 7.35~7.62 (17H, m), 7.16 (1H, t) |
| 1-173 | δ = 8.55 (1H, d), 8.36 (4H, m), 8.19~8.22 (2H, m), |
| | 8.04~8.10 (3H, m), 7.90~7.94 (2H, m), 7.35~7.62 (17H, |
| | m), 7.16~7.20 (2H, m) |
| 1-177 | δ = 8.55 (1H, d), 8.36 (2H, m), 8.19~8.22 (2H, m), |
| | 8.04~8.10 (3H, m), 7.90~7.96 (4H, m), 7.75 (2H, d), |
| | 7.35~7.62 (17H, m), 7.16~7.25 (4H, m) |
| 1-182 | δ = 8.55 (1H, d), 8.36 (4H, m), 8.19~8.22 (2H, m), |
| | 8.04~8.10 (3H, m), 7.90~7.94 (2H, m), 7.80 (1H, d), |
| | 7.35~7.62 (16H, m), 7.16~7.20 (2H, t) |
| 1-185 | δ = 8.65 (1H, d), 8.55 (1H, d), 8.30~8.36 (5H, m), 8.19 |
| | (1H, d), 7.94 (1H, d), 7.84 (1H, d), 7.74 (1H, d), |
| | 7.35~7.62 (18H, m), 7.16~7.20 (2H, t) |
| 1-189 | δ = 8.65 (1H, d), 8.55 (1H, d), 8.30~8.36 (3H, m), 8.19 |
| | (1H, d), 7.94~7.96 (3H, m), 7.84 (1H, d), 7.74~7.75 |
| | (3H, d), 7.16~7.62 (22H, m) |
| 1-199 | δ = 8.65 (1H, d), 8.55 (1H, d), 8.30~8.36 (5H, m), 8.19 |
| | (1H, d), 7.94 (1H, d), 7.84 (1H, d), 7.74 (1H, d), |
| | 7.31~7.62 (17H, m), 7.13~7.20 (3H, m) |
| 1-201 | δ = 8.62 (1H, d), 8.55 (1H, d), 8.36 (4H, m), 8.19~8.22 |
| | (2H, m), 7.94 (1H, d), 7.84 (1H, d), 7.74 (2H, m), |
| | 7.48~7.62 (15H, m), 7.31~7.35 (2H, m), 7.16~7.20 (2H, |
| | m) |
| 1-203 | δ = 8.62 (1H, d), 8.55 (1H, d), 8.31~8.36 (5H, m), 8.22 |
| | (1H, d), 7.91~7.94 (2H, m), 7.84 (1H, d), 7.74~7.75 |
| | (5H, m), 7.31~7.62 (18H, m), 7.16 (1H, t) |
| 1-212 | δ = 8.62 (1H, d), 8.55 (1H, d), 8.36 (2H, m), 8.22 (1H, |
| | d), 7.89~7.99 (5H, m), 7.74~7.77 (7H, m), 7.25~7.62 |
| | (18H, m), 7.16 (1H, t) |
| 1-214 | δ = 8.62 (1H, d), 8.55 (1H, d), 8.31~8.36 (5H, m), 8.22 |
| | (1H, d), 7.91~7.94 (2H, m), 7.74~7.75 (5H, m), |
| | 7.31~7.62 (18H, m), 7.16~7.19 (2H, m) |
| 1-218 | δ = 8.55 (1H, d), 8.30~8.36 (5H, m), 8.13 (1H, d), |
| | 7.84~7.99 (5H, m), 7.74~7.77 (4H, m), 7.31~7.62 (18H, |
| | m), 7.16 (1H, t) |
| 1-221 | δ = 8.55 (1H, d), 8.30~8.36 (3H, m), 8.13~8.19 (2H, m), |
| | 7.84~7.96 (5H, m), 7.74~7.75 (2H, d), 7.16~7.62 (21H, |
| | m) |
| 1-247 | δ = deuterium content of 100% with no ¹H NMR peak |
| 1-249 | δ = 8.65 (1H, d), 8.55 (1H, d), 8.30~8.36 (5H, m), 8.19 |
| | (1H, d), 8.05 (1H, d), 7.89~7.94 (2H, m), 7.44~7.63 |
| | (18H, m), 7.16~7.20 (2H, t) |
| 1-253 | δ = 8.65 (1H, d), 8.45 (1H, d), 8.30~8.36 (3H, m), 8.19 |
| | (1h, d), 7.94 (1H, d), 7.58~7.96 (4H, m), 7.75 (2H, d), |
| | 7.35~7.96 (18H, m), 7.16~7.25 (4H, m) |
| 1-257 | δ = 8.65 (1H, d), 8.55 (1H, d), 8.30~8.36 (5H, m), 8.19 |
| | (1H, d), 7.86~7.94 (3H, m), 7.78 (1H, s), 7.35~7.63 |
| | (16H, m), 7.16~7.20 (2H, t) |
| 1-261 | δ = 8.65 (1H, d), 8.55 (1H, d), 8.30~8.36 (5H, m), 8.19 |
| | (1H, d), 8.05 (1h, d), 7.89~7.94 (2H, m), 7.35~7.63 |
| | (18H, m), 7.16~7.20 (2H, m) |
| 1-263 | δ = 8.62 (1H, d), 8.55 (1H, d), 8.36 (4H, m), 8.22 (1H, |
| | d), 7.89~8.05 (5H, m), 7.74-7.77 (4H, m), 7.35~7.62 |
| | (18H, m), 7.16 (1H, t) |
| 1-265 | δ = 8.55~8.62 (2H, m), 8.36 (2H, m), 8.19~8.22 (2H, m), |
| | 8.05 (1H, d), 7.89~7.96 (4H, m), 7.74~7.75 (3H, d), |
| | 7.35~7.63 (17H, m), 7.16~7.25 (4H, m) |
| 1-275 | δ = 8.62 (1H, d), 8.55 (1H, d), 8.36 (4H, m), 8.19~8.22 |
| | (2H, m), 8.05 (1H, d), 7.89~7.94 (2H, m), 7.74 (1H, s), |
| | 7.45~7.63 (15H, m), 7.33~7.35 (2H, m), 7.16~7.20 (2H, |
| | t) |
| 1-279 | δ = 8.55 (1H, d), 8.30~8.36 (3H, m), 8.13~8.19 (2H, m), |
| | 8.05 (1H, d), 7.89~7.96 (5H, m), 7.75 (2H, d), |
| | 7.35~7.63 (17H, m), 7.16~7.25 (4H, m) |
| 1-282 | δ = 8.55 (1H, d), 8.30~8.36 (5H, m), 8.13~8.19 (2H, m), |
| | 7.86~7.94 (4H, m), 7.78 (1H, s), 7.45~7.78 (15H, m), |
| | 7.35 (1H, t), 7.16~7.20 (2H, t) |
| 1-287 | δ = 8.55 (1H, d), 8.30~8.36 (5H, m), 8.13~8.19 (2H, m), |
| | 8.05 (1H, d), 7.89~7.94 (3H, m), 7.45~7.63 (15H, m), |
| | 7.33~7.35 (2H, m), 7.16~7.20 (2H, t) |
| 1-290 | δ = 8.55 (1H, d), 8.36 (4H, m), 8.22 (1H, d), 7.89~8.05 |
| | (6H, m), 7.75~7.77 (3H, m), 7.41~7.63 (19H, m), 7.16 |
| | (1H, t) |
| 1-294 | δ = 8.55 (1H, d), 8.36~8.38 (3H, m), 8.19~8.22 (2H, m), |
| | 8.04~8.05 (2H, d), 7.89~7.94 (3H, m), 7.73~7.75 (3H, |
| | m), 7.35~7.63 (19H, m), 7.16~7.20 (2H, t) |
| 1-298 | δ = 8.65 (1H, d), 8.55 (1H, d), 8.30~8.36 (5H, m), |
| | 7.84~7.99 (4H, m), 7.75~7.77 (3H, m), 7.35~7.62 (19H, |
| | m), 7.25 (1H, d), 7.16 (1H, t) |
| 1-302 | δ = 8.65 (1H, d), 8.55 (1H, d), 7.30~7.58 (4H, m), 7.19 |
| | (1H, d), 7.84~7.94 (2H, m), 7.84 (1H, d), 7.73~7.75 |
| | (3H, m), 7.35~7.62 (19H, m), 7.16~7.25 (3H, m) |
| 1-309 | δ = 8.65 (1H, d), 8.55 (1H, d), 8.30~8.36 (5H, m), 8.19 |
| | (1H, d), 7.94 (1H, d), 7.84 (1H, d), 7.35~7.62 (17H, |
| | m), 7.13~7.25 (4H, m) |
| 1-310 | δ = 8.62 (1H, d), 8.55 (1H, d), 8.36 (4H, m), 8.19~8.22 |
| | (2H, m), 7.94 (1H, d), 7.84 (1H, d), 7.74 (1H, s), |
| | 7.48~7.62 (16H, m), 7.35 (1H, t), |
| 1-313 | δ = 8.62 (1H, d), 8.55 (1H, d), 8.36 (2H, m), 8.19~8.22 |
| | (2H, m), 7.94~7.96 (3H, m), 7.84 (1H, d), 7.74~7.75 |
| | (3H, d), 7.35~7.62 (17H, m), 7.16~7.25 (5H, m) |
| 1-323 | δ = 8.62 (1H, d), 8.55 (1H, d), 8.36 (4H, m), 8.19~8.22 |
| | (2H, m), 7.94 (1H, d), 7.84 (1H, d), 7.74 (1H, s), |
| | 7.50~7.62 (15H, m), 7.35 (1H, t), 7.13~7.20 (4H, m) |
| 1-324 | δ = 8.55 (1H, d), 8.30~8.36 (4H, m), 8.13~8.19 (2H, m), |
| | 7.84~7.94 (3H, m), 7.48~7.62 (16H, m), 7.35 (1H, t), |
| | 7.16~7.25 (3H, m) |
| 1-327 | δ = 8.55 (1H, d), 8.30~8.36 (3H, m), 8.13~8.19 (2H, m), |
| | 7.84~7.96 (5H, m), 7.75 (2H, d), 7.35~7.62 (17H, m), |
| | 7.16~7.25 (5H, m) |
| 1-330 | δ = 8.55 (1H, d), 8.30~8.36 (5H, m), 8.13~8.19 (2H, m), |
| | 7.89~7.94 (2H, m), 7.50~7.62 (16H, m), 7.35 (1H, t), |
| | 7.16~7.25 (3H, m) |
| 1-335 | δ = 8.55 (1H, d), 8.36 (4H, m), 8.13~8.19 (2H, m), |
| | 7.84~7.94 (3H, m), 7.50~7.62 (15H, m), 7.35 (1H, t), |
| | 7.13~7.25 (4H, m) |
| 1-339 | δ = 8.55 (1H, d), 8.31~8.36 (5H, m), 8.22 (1H, d), 8.04 |
| | (1H, d), 7.91~7.94 (2H, m), 7.84 (1H, d), 7.74~7.75 |
| | (3H, m), 7.35~7.62 (19H, m), 7.25 (1H, d), 7.16 (1H, t) |
| 1-348 | δ = deuterium content of 100% with no ¹H NMR peak |
| 1-350 | δ = 8.51~8.55 (3H, m), 8.36 (4H, m), 7.89~8.05 (5H, m), |
| | 7.35~7.77 (22H, m), 7.16 (1H, t) |
| 1-358 | δ = 8.51~8.55 (3H, m), 8.36 (4H, m), 8.19 (1H, d), 8.08 |
| | (1H, d), 7.92~7.94 (2H, m), 7.50~7.74 (16H, m), |
| | 7.35~7.39 (2H, m), 7.16~7.20 (2H, t) |
| 1-360 | δ = 8.51~8.55 (2H, m), 8.36 (2H, m), 8.08 (1H, d), |
| | 7.89~7.99 (6H, m), 7.35~7.80 (19H, m), 7.25 (2H, d), |
| | 7.16 (1H, t) |
| 1-366 | δ = 8.51~8.55 (2H, m), 8.36 (4H, m), 8.20~8.24 (2H, m), |
| | 7.89~8.05 (5H, m), 7.74~7.77 (4H, m), 7.35~7.62 (17H, |
| | m), 7.16 (1H, t) |
| 1-369 | δ = 8.51~8.55 (2H, m), 8.36 (2H, m), 8.19~8.24 (3H, m), |
| | 8.05 (1H, d), 7.94~7.96 (3H, m), 7.74~7.75 (3H, d), |
| | 7.35~7.62 (16H, m), 7.16~7.25 (4H, m) |
| 1-375 | δ = 8.51~8.55 (2H, m), 8.36 (4H, m), 8.20~8.24 (2H, m), |
| | 8.08 (1H, d), 7.89~7.99 (4H, m), 7.74~7.80 (5H, m), |
| | 7.35~7.62 (16H, m), 7.16 (1H, t) |
| 1-383 | δ = 8.51~8.55 (2H, m), 8.31~8.36 (5H, m), 8.12 (1H, s), |
| | 7.91~8.05 (5H, m), 7.74-7.75 (4H, m), 7.35~7.62 (17H, |
| | m), 7.16 (1H, t) |
| 1-388 | δ = 8.51~8.55 (2H, m), 8.19 (1H, d), 8.12 (1H, s), |
| | 7.92~8.05 (8H, m), 7.74~7.75 (5H, d), 7.35~7.62 (16H, |
| | m), 7.16~7.25 (6H, m) |
| 1-389 | δ = 8.55 (1H, d), 8.36 (4H, m), 8.12~8.19 (2H, m), |
| | 7.92~7.99 (3H, m), 7.86 (1H, s), 7.78 (1H, s), |
| | 7.35~7.58 (16H, m), 7.16~7.20 (2H, t) |
| 1-392 | δ = 8.51~8.55 (2H, m), 8.36 (2H, m), 8.08~8.12 (2H, m), |
| | 7.89~7.99 (7H, m), 7.75~7.80 (6H, m), 7.35~7.62 (15H, |
| | m), 7.25 (2H, d), 7.50 (1H, t) |
| 1-397 | δ = 8.51 (1H, m), 8.36 (4H, m), 8.19 (1H, d), 8.03~8.05 |
| | (2H, m), 7.94 (2H, d), 7.50~7.62 (16H, m), 7.35~7.39 |
| | (2H, m), 7.16~7.20 (2H, t) |
| 1-399 | δ = 8.51~8.55 (2H, m), 8.31~8.36 (5H, m), 8.03~8.05 |
| | (2H, m), 7.91~7.94 (3H, m), 7.35~7.75 (22H, m), 7.16 |
| | (1H, t) |
| 1-404 | δ = 8.51~8.55 (2H, m), 8.19 (1H, d), 7.94~8.05 (8H, m), |
| | 7.35~7.75 (22H, m), 7.16~7.25 (6H, m) |
| 1-410 | δ = 8.51~8.55 (2H, m), 8.36 (4H, m), 8.08 (1H, d), |
| | 7.84~7.99 (5H, m), 7.74~7.77 (4H, m), 7.35~7.62 (18H, |
| | m), 7.16 (1H, t) |
| 1-413 | δ = 8.51~8.55 (12H, m), 8.36 (2H, m), 8.19 (1H, d), |
| | 8.08 (1H, d), 7.84~7.96 (5H, m), 7.74~7.75 (3H, d) |
| | 7.35~7.62 (17H, m), 7.16~7.25 (4H, m) |
| 1-418 | δ = 8.51~8.55 (2H, m), 8.36 (4H, m), 8.19 (1H, d), 8.08 |
| | (1H, d), 7.94~7.88 (2H, m), 7.74 (1H, d), 7.50~7.62 |
| | (16H, m), 7.35~7.39 (2H, m), 7.16~7.20 (2H, t) |
| 1-421 | δ = 8.51~8.55 (2H, m), 8.36 (4H, m), 8.19 (1H, d), 8.08 |
| | (1H, d), 7.88~7.94 (2H, m), 7.74 (1H, d), 7.50~7.62 |
| | (15H, m), 7.35~7.39 (2H, m), 7.16~7.20 (3H, m) |
| 1-427 | δ = 8.51~8.55 (2H, m), 8.31~8.36 (5H, m), 8.03 (1H, s), |
| | 7.91~7.94 (2H, m), 7.74~7.84 (7H, m), 7.35~7.62 (17H, |
| | m), 7.16 (1H, t) |
| 1-430 | δ = 8.62 (1H, d), 8.55 (1H, d), 8.36~8.38 (3H, m), |
| | 8.19~8.22 (2H, m), 7.94 (3H, m), 7.84 (1H, d), |
| | 7.73~7.75 (4H, m), 7.41~7.62 (18H, m), 7.16~7.20 (2H, |
| | t) |
| 1-436 | δ = 8.51~8.55 (2H, m), 8.36 (2H, m), 7.89~8.03 (6H, m), |
| | 7.74~7.82 (8H, m), 7.35~7.62 (18H, m), 7.25 (2H, d), |
| | 7.16 (1H, t) |
| 1-441 | δ = 8.51~8.55 (2H, m), 8.36 (4H, m), 8.19 (1H, d), |
| | 7.74~7.94 (6H, m), 7.48~7.62 (14H, m), 8.35~8.39 (2H, |
| | m), 7.16~7.20 (2H, t) |
| 1-445 | δ = 8.51~8.55 (2H, m), 8.36 (2H, m), 8.19 (1H, d), |
| | 7.74~7.96 (10H, m), 7.35~7.94 (16H, m), 7.16~7.25 (4H, |
| | m) |
| 1-450 | δ = 8.51~8.55 (2H, m), 8.36 (4H, m), 8.19 (1H, d), |
| | 7.74~7.94 (5H, m), 7.50~7.62 (15H, m), 7.35~7.39 (2H, |
| | t), 7.16~7.20 (2H, t) |
| 1-452 | δ = 8.51~8.55 (2H, m), 8.36 (2H, m), 7.74~7.99 (14H, |
| | m), 7.35~7.62 (18H, m), 7.25 (2H, d), 7.16 (1H, t) |
| 1-469 | δ = deuterium content of 100% with no ¹H NMR peak |
| 2-3 | δ = 8.55 (1H, d), 8.30 (1H, d), 8.13~8.21 (3H, m), |
| | 7.89~7.99 (4H, m), 7.35~7.68 (17H, m), 7.16~7.20 (2H, |
| | t) |
| 2-31 | δ = 8.55 (1H, d), 8.30 (1H, d), 8.13~8.21 (4H, m), |
| | 7.89~7.99 (4H, m), 7.35~7.77 (20H, m), 7.16~7.20 (2H, |
| | t) |
| 2-32 | δ = 8.55 (1H, d), 8.30 (1H, d), 8.13~7.21 (3H, m), |
| | 7.89~7.99 (8H, m), 7.35~7.77 (17H, m), 7.16~7.20 (2H, |
| | t) |
| 2-44 | δ = 8.55 (1H, d), 8.30 (1H, d), 8.13~8.19 (2H, m), |
| | 7.89~7.99 (12H, m), 7.75~7.77 (5H, m), 7.58 (1H, d), |
| | 7.35~7.49 (8H, m), 7.16~7.25 (6H, m) |
| 2-98 | δ = 8.55 (1H, d), 8.30 (1H, d), 8.13~8.19 (2H, m), |
| | 7.89~7.94 (4H, m), 7.77 (1H, d), 7.50~7.58 (2H, m), |
| | 7.35 (1H, t), 7.16~7.20 (2H, m) |
| 2-101 | δ = 8.55 (1H, d), 8.30 (1H, d), 8.13~8.19 (2H, m), |
| | 7.89~7.99 (4H, m), 7.77 (1H, d), 7.50~7.58 (2H, m), |
| | 7.35 (1H, t), 7.16~7.20 (2H, m) |
| 2-110 | δ = 8.55 (1H, d), 8.30 (1H, d), 8.13~8.19 (2H, m), |
| | 7.89~7.99 (4H, m), 7.77 (1H, d), 7.50~7.58 (2H, m), |
| | 7.35 (1H, t), 7.16~7.20 (2H, m) |
| 2-122 | δ = 7.91~7.92 (8H, m), 7.75 (4H, d), 7.41~7.49 (6H, m) |
| 2-130 | δ = 8.21 (1H, s), 7.91~7.94 (5H, m), 7.60~7.75 (9H, m), |
| | 7.41~7.49 (7H, m) |
| 2-146 | δ = deuterium content of 100% with no ¹H NMR peak |
| 2-149 | δ = deuterium content of 100% with no ¹H NMR peak |
| 2-154 | δ = deuterium content |
| 2-158 | δ = deuterium content of 100% with no ¹H NMR peak |

TABLE 8

| Compound | FD-Mass | Compound | FD-Mass |
|---|---|---|---|
| 1-1 | m/z = 745.90 | 1-5 | m/z = 822.00 |
| | (C51H31N5S = 745.23) | | (C57H35N5S = 821.26) |
| 1-12 | m/z = 898.10 | 1-22 | m/z = 822.00 |
| | (C63H39N5S = 897.29) | | (C57H35N5S = 821.26) |
| 1-32 | m/z = 822.00 | 1-38 | m/z = 822.00 |
| | (C57H35N5S = 821.26) | | (C57H35N5S = 821.26) |
| 1-42 | m/z = 745.90 | 1-46 | m/z = 822.00 |
| | (C51H31N5S = 745.23) | | (C57H35N5S = 821.26) |
| 1-50 | m/z = 822.00 | 1-53 | m/z = 822.00 |
| | (C57H35N5S = 821.26) | | (C57H35N5S = 821.26) |
| 1-60 | m/z = 898.10 | 1-63 | m/z = 805.94 |
| | (C63H39N5S = 897.29) | | (C57H35N5O = 805.28) |
| 1-65 | m/z = 805.94 | 1-72 | m/z = 882.04 |
| | (C57H35N5O = 805.28) | | (C63H29N5O = 881.32) |
| 1-79 | m/z = 805.94 | 1-81 | m/z = 805.94 |
| | (C57H35N5O = 805.28) | | (C57H35N5O = 805.28) |
| 1-88 | m/z = 882.04 | 1-91 | m/z = 729.84 |
| | (C63H29N5O = 881.32) | | (C51H31N5O = 729.25) |
| 1-97 | m/z = 805.94 | 1-100 | m/z = 882.04 |
| | (C57H35N5O = 805.28) | | (C63H29N5O = 881.32) |
| 1-102 | m/z = 729.84 | 1-108 | m/z = 805.94 |
| | (C51H31N5O = 729.25) | | (C57H35N5O = 805.28) |
| 1-113 | m/z = 805.94 | 1-120 | m/z = 882.04 |
| | (C57H35N5O = 805.28) | | (C63H29N5O = 881.32) |
| 1-121 | m/z = 857.22 | 1-125 | m/z = 745.90 |
| | (C57D35N5S = 856.48) | | (C51H31N5S = 745.23) |
| 1-126 | m/z = 822.00 | 1-129 | m/z = 822.00 |
| | (C57H35N5S = 821.26) | | (C57H35N5S = 821.26) |
| 1-132 | m/z = 898.10 | 1-141 | m/z = 745.90 |
| | (C63H39N5S = 897.29) | | (C51H31N5S = 745.23) |
| 1-143 | m/z = 822.00 | 1-145 | m/z = 822.00 |
| | (C57H35N5S = 821.26) | | (C57H35N5S = 821.26) |
| 1-150 | m/z = 745.90 | 1-154 | m/z = 822.00 |
| | (C51H31N5S = 745.23) | | (C57H35N5S = 821.26) |
| 1-158 | m/z = 822.00 | 1-173 | m/z = 745.90 |
| | (C57H35N5S = 821.26) | | (C51H31N5S = 745.23) |
| 1-177 | m/z = 822.00 | 1-182 | m/z = 745.90 |
| | (C57H35N5S = 821.26) | | (C51H31N5S = 745.23) |
| 1-185 | m/z = 729.84 | 1-189 | m/z = 805.94 |
| | (C51H31N5O = 729.25) | | (C57H35N5O = 805.28) |
| 1-199 | m/z = 729.84 | 1-201 | m/z = 729.84 |
| | (C51H31N5O = 729.25) | | (C51H31N5O = 729.25) |
| 1-203 | m/z = 805.94 | 1-212 | m/z = 882.04 |
| | (C57H35N5O = 805.28) | | (C63H39N5O = 881.32) |
| 1-214 | m/z = 805.94 | 1-218 | m/z = 805.94 |
| | (C57H35N5O = 805.28) | | (C57H35N5O = 805.28) |
| 1-221 | m/z = 805.94 | 1-247 | m/z = 777.09 |
| | (C57H35N5O = 805.28) | | (C51D31N5S = 776.42) |
| 1-249 | m/z = 745.90 | 1-253 | m/z = 822.00 |
| | (C51H31N5S = 745.23) | | (C57H35N5S = 821.26) |
| 1-257 | m/z = 745.90 | 1-261 | m/z = 745.90 |
| | (C51H31N5S = 745.23) | | (C51H31N5S = 745.23) |
| 1-263 | m/z = 822.00 | 1-265 | m/z = 822.00 |
| | (C57H35N5S = 821.26) | | (C57H35N5S = 821.26) |
| 1-275 | m/z = 745.90 | 1-279 | m/z = 822.00 |
| | (C51H31N5S = 745.23) | | (C57H35N5S = 821.26) |
| 1-282 | m/z = 745.90 | 1-287 | m/z = 745.90 |
| | (C51H31N5S = 745.23) | | (C51H31N5S = 745.23) |
| 1-290 | m/z = 822.00 | 1-294 | m/z = 822.00 |
| | (C57H35N5S = 821.26) | | (C57H35N5S = 821.26) |
| 1-298 | m/z = 805.94 | 1-302 | m/z = 805.94 |
| | (C57H35N5O = 805.28) | | (C57H35N5O = 805.28) |
| 1-309 | m/z = 729.84 | 1-310 | m/z = 729.84 |
| | (C51H35N5O = 729.25) | | (C51H31N5O = 729.25) |
| 1-313 | m/z = 805.94 | 1-323 | m/z = 729.84 |
| | (C57H35N5O = 805.28) | | (C51H35N5O = 729.25) |
| 1-324 | m/z = 729.84 | 1-327 | m/z = 805.94 |
| | (C51H35N5O = 729.25) | | (C57H35N5O = 805.28) |
| 1-330 | m/z = 729.84 | 1-335 | m/z = 729.84 |
| | (C51H35N5O = 729.25) | | (C51H35N5O = 729.25) |
| 1-339 | m/z = 805.94 | 1-348 | m/z = 761.03 |
| | (C57H35N5O = 805.28) | | (C51D31N5O = 760.45) |
| 1-350 | m/z = 822.00 | 1-358 | m/z = 745.90 |
| | (C57H35N5S = 821.26) | | (C51H31N5S = 745.23) |
| 1-360 | m/z = 898.10 | 1-366 | m/z = 822.00 |
| | (C63H39N5S = 897.29) | | (C57H35N5S = 821.26) |
| 1-369 | m/z = 822.00 | 1-375 | m/z = 822.00 |
| | (C57H35N5S = 821.26) | | (C57H35N5S = 821.26) |
| 1-383 | m/z = 822.00 | 1-388 | m/z = 898.10 |
| | (C57H35N5S = 821.26) | | (C63H39N5S = 897.29) |
| 1-389 | m/z = 745.90 | 1-392 | m/z = 898.10 |
| | (C51H31N5S = 745.23) | | (C63H39N5S = 897.29) |
| 1-397 | m/z = 745.90 | 1-399 | m/z = 822.00 |
| | (C51H31N5S = 745.23) | | (C57H35N5S = 821.26) |
| 1-404 | m/z = 898.10 | 1-410 | m/z = 805.94 |
| | (C63H39N5S = 897.29) | | (C57H35N5O = 805.28) |
| 1-413 | m/z = 805.94 | 1-418 | m/z = 729.84 |
| | (C57H35N5O = 805.28) | | (C51H31N5O = 729.25) |
| 1-421 | m/z = 729.84 | 1-427 | m/z = 805.94 |
| | (C51H31N5O = 729.25) | | (C57H35N5O = 805.28) |
| 1-430 | m/z = 805.94 | 1-436 | m/z = 882.04 |
| | (C57H35N5O = 805.28) | | (C63H39N5O = 881.32) |
| 1-441 | m/z = 729.84 | 1-445 | m/z = 805.94 |
| | (C51H31N5O = 729.25) | | (C57H35N5O = 805.28) |
| 1-450 | m/z = 729.84 | 1-452 | m/z = 882.04 |
| | (C51H31N5O = 729.25) | | (C63H39N5O = 881.32) |
| 1-469 | m/z = 856.21 | 2-3 | m/z = 560.70 |
| | (C57HD34N5S = 855.47) | | (C42H28N2 = 560.23) |
| 2-31 | m/z = 636.80 | 2-32 | m/z = 636.80 |
| | (C48H32N2 = 636.26) | | (C48H32N2 = 636.26) |
| 2-44 | m/z = 712.90 | 2-98 | m/z = 654.91 |
| | (C54H36N2 = 712.29) | | (C48H14D18N2 = 654.37) |
| 2-101 | m/z = 654.91 | 2-110 | m/z = 735.03 |
| | (C48H14D18N2 = 654.37) | | (C54H14D22N2 = 734.43) |
| 2-122 | m/z = 650.88 | 2-130 | m/z = 726.98 |
| | (C48H18D14N2 = 650.34) | | (C54H22D14N2 = 726.38) |
| 2-146 | m/z = 668.99 | 2-149 | m/z = 668.99 |
| | (C48D32N2 = 668.46) | | (C48D32N2 = 668.46) |
| 2-154 | m/z = 749.12 | 2-158 | m/z = 749.12 |
| | (C54D36N2 = 748.51) | | (C54D36N2 = 748.51) |

### EXPERIMENTAL EXAMPLES

### Experimental Example 1

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

The hole injection layer 4,4′,4″-tris[2-naphthyl(phenyl)amino]triphenylamine (2-TNATA) and the hole transport layer N,N′-diphenyl-(1,1′-biphenyl)-4,4′-diamine (NPB), which are common layers, were formed on the ITO transparent electrode (positive electrode).

A light emitting layer was thermally vacuum deposited thereon as follows. The light emitting layer was deposited to have a thickness of 400 Å by using a compound described in the following Table 9 or Table 10 as a host and tris(2-phenylpyridine)iridium (Ir(ppy)₃) as a green phosphorescent dopant to dope the host with Ir(ppy)₃ in an amount of 7%. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁶ to 10⁻⁸ torr for each material, and used for the manufacture of OLED.

<Driving Voltage and Light Emitting Efficiency of Organic Electroluminescence Device>

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

For reference, the case of Table 9 corresponds to the case of including the heterocyclic compound of Chemical Formula 1 and the heterocyclic compound of Chemical Formula 2, and the case of Table 10 corresponds to the case of including the heterocyclic compound of Chemical Formula 1 alone.

TABLE 9

| | Light | | | | | |
|---|---|---|---|---|---|---|
| | emitting | | Driving | effi- | Color | Service |
| | layer | | voltage | ciency | coor- | life |
| | Compound | Ratio | (V) | (cd/A) | dinate | (T₉₀) |
| Comparative | 3-1:2-146 | 1:1 | 5.32 | 62.9 | Green | 168 |
| Example 1 | | | | | | |
| Comparative | | 1:2 | 5.39 | 62.1 | Green | 171 |
| Example 2 | | | | | | |
| Comparative | | 1:3 | 5.42 | 61.3 | Green | 175 |
| Example 3 | | | | | | |
| Comparative | 3-2:2-154 | 1:1 | 4.74 | 70.2 | Green | 183 |
| Example 4 | | | | | | |
| Comparative | | 1:2 | 4.83 | 69.3 | Green | 192 |
| Example 5 | | | | | | |
| Comparative | | 1:3 | 4.88 | 68.1 | Green | 200 |
| Example 6 | | | | | | |
| Comparative | 3-3:2-158 | 1:1 | 5.33 | 63.7 | Green | 165 |
| Example 7 | | | | | | |
| Comparative | | 1:2 | 5.37 | 62.6 | Green | 169 |
| Example 8 | | | | | | |
| Comparative | | 1:3 | 5.46 | 61.5 | Green | 173 |
| Example 9 | | | | | | |
| Comparative | 3-4:2-149 | 1:1 | 5.41 | 63.8 | Green | 150 |
| Example 10 | | | | | | |
| Comparative | | 1:2 | 5.44 | 62.6 | Green | 152 |
| Example 11 | | | | | | |
| Comparative | | 1:3 | 5.53 | 60.1 | Green | 155 |
| Example 12 | | | | | | |
| Comparative | 3-5:2-146 | 1:1 | 4.62 | 69.2 | Green | 180 |
| Example 13 | | | | | | |
| Comparative | | 1:2 | 4.68 | 68.5 | Green | 183 |
| Example 14 | | | | | | |
| Comparative | | 1:3 | 4.73 | 67.1 | Green | 188 |
| Example 15 | | | | | | |
| Comparative | 3-6:2-146 | 1:1 | 5.32 | 62.5 | Green | 162 |
| Example 16 | | | | | | |
| Comparative | | 1:2 | 5.38 | 62.2 | Green | 168 |
| Example 17 | | | | | | |
| Comparative | | 1:3 | 5.40 | 61.0 | Green | 172 |
| Example 18 | | | | | | |
| Example 1 | 1-1:2-154 | 1:1 | 2.35 | 143.2 | Green | 562 |
| Example 2 | | 1:2 | 2.46 | 142.6 | Green | 573 |
| Example 3 | | 1:3 | 2.55 | 142.0 | Green | 580 |
| Example 4 | 1-5:2-158 | 1:1 | 2.38 | 140.3 | Green | 552 |
| Example 5 | | 1:2 | 2.49 | 139.7 | Green | 563 |
| Example 6 | | 1:3 | 2.58 | 138.2 | Green | 571 |
| Example 7 | 1-12:2-158 | 1:1 | 2.13 | 144.6 | Green | 546 |
| Example 8 | | 1:2 | 2.26 | 143.2 | Green | 553 |
| Example 9 | | 1:3 | 2.39 | 142.8 | Green | 563 |
| Example 10 | 1-22:2-149 | 1:1 | 2.67 | 145.3 | Green | 544 |
| Example 11 | | 1:2 | 2.78 | 144.6 | Green | 563 |
| Example 12 | | 1:3 | 2.85 | 143.7 | Green | 571 |
| Example 13 | 1-32:2-154 | 1:1 | 2.66 | 144.7 | Green | 538 |
| Example 14 | | 1:2 | 2.73 | 143.2 | Green | 549 |
| Example 15 | | 1:3 | 2.85 | 142.6 | Green | 558 |
| Example 16 | 1-38:2-158 | 1:1 | 2.64 | 143.7 | Green | 521 |
| Example 17 | | 1:2 | 2.69 | 142.6 | Green | 538 |
| Example 18 | | 1:3 | 2.71 | 141.2 | Green | 559 |
| Example 19 | 1-42:2-146 | 1:1 | 2.55 | 140.6 | Green | 487 |
| Example 20 | | 1:2 | 2.63 | 138.7 | Green | 493 |
| Example 21 | | 1:3 | 2.71 | 138.2 | Green | 503 |
| Example 22 | 1-50:2-149 | 1:1 | 2.44 | 144.6 | Green | 513 |
| Example 23 | | 1:2 | 2.53 | 143.2 | Green | 526 |
| Example 24 | | 1:3 | 2.65 | 142.8 | Green | 538 |
| Example 25 | 1-53:2-149 | 1:1 | 2.87 | 143.2 | Green | 528 |
| Example 26 | | 1:2 | 2.93 | 142.6 | Green | 533 |
| Example 27 | | 1:3 | 3.01 | 141.7 | Green | 548 |
| Example 28 | 1-60:2-146 | 1:1 | 2.77 | 145.2 | Green | 457 |
| Example 29 | | 1:2 | 2.83 | 144.7 | Green | 463 |
| Example 30 | | 1:3 | 2.91 | 143.2 | Green | 472 |
| Example 31 | 1-63:2-154 | 1:1 | 2.62 | 138.7 | Green | 455 |
| Example 32 | | 1:2 | 2.68 | 137.5 | Green | 467 |
| Example 33 | | 1:3 | 2.70 | 136.2 | Green | 479 |
| Example 34 | 1-65:2-158 | 1:1 | 2.56 | 137.5 | Green | 522 |
| Example 35 | | 1:2 | 2.68 | 136.2 | Green | 538 |
| Example 36 | | 1:3 | 2.76 | 135.4 | Green | 549 |
| Example 37 | 1-72:2-154 | 1:1 | 2.44 | 133.2 | Green | 530 |
| Example 38 | | 1:2 | 2.53 | 132.8 | Green | 535 |
| Example 39 | | 1:3 | 2.67 | 131.7 | Green | 546 |
| Example 40 | 1-79:2-158 | 1:1 | 3.05 | 134.6 | Green | 521 |
| Example 41 | | 1:2 | 3.12 | 133.2 | Green | 537 |
| Example 42 | | 1:3 | 3.28 | 132.8 | Green | 544 |
| Example 43 | 1-81:2-146 | 1:1 | 3.12 | 135.6 | Green | 502 |
| Example 44 | | 1:2 | 3.23 | 134.7 | Green | 513 |
| Example 45 | | 1:3 | 3.37 | 133.5 | Green | 526 |
| Example 46 | 1-88:2-154 | 1:1 | 3.08 | 136.2 | Green | 519 |
| Example 47 | | 1:2 | 3.15 | 135.7 | Green | 523 |
| Example 48 | | 1:3 | 3.26 | 134.2 | Green | 534 |
| Example 49 | 1-91:2-154 | 1:1 | 2.56 | 139.5 | Green | 527 |
| Example 50 | | 1:2 | 2.63 | 138.4 | Green | 538 |
| Example 51 | | 1:3 | 2.77 | 137.1 | Green | 544 |
| Example 52 | 1-97:2-149 | 1:1 | 2.51 | 138.5 | Green | 526 |
| Example 53 | | 1:2 | 2.56 | 137.2 | Green | 537 |
| Example 54 | | 1:3 | 2.83 | 136.1 | Green | 542 |
| Example 55 | 1-100:2-158 | 1:1 | 2.41 | 137.2 | Green | 487 |
| Example 56 | | 1:2 | 2.57 | 136.5 | Green | 493 |
| Example 57 | | 1:3 | 2.69 | 135.8 | Green | 499 |
| Example 58 | 1-102:2-154 | 1:1 | 3.12 | 139.2 | Green | 493 |
| Example 59 | | 1:2 | 3.18 | 138.5 | Green | 499 |
| Example 60 | | 1:3 | 3.25 | 137.1 | Green | 513 |
| Example 61 | 1-108:2-146 | 1:1 | 2.45 | 134.2 | Green | 482 |
| Example 62 | | 1:2 | 2.62 | 133.6 | Green | 493 |
| Example 63 | | 1:3 | 2.66 | 132.7 | Green | 507 |
| Example 64 | 1-113:2-154 | 1:1 | 2.38 | 138.8 | Green | 487 |
| Example 65 | | 1:2 | 2.41 | 137.2 | Green | 493 |
| Example 66 | | 1:3 | 2.56 | 136.5 | Green | 499 |
| Example 67 | 1-120:2-146 | 1:1 | 3.10 | 137.2 | Green | 473 |
| Example 68 | | 1:2 | 3.15 | 136.2 | Green | 478 |
| Example 69 | | 1:3 | 3.21 | 135.5 | Green | 482 |
| Example 70 | 1-121:2-98 | 1:1 | 3.11 | 134.6 | Green | 455 |
| Example 71 | | 1:2 | 3.23 | 133.2 | Green | 462 |
| Example 72 | | 1:3 | 3.34 | 132.8 | Green | 469 |
| Example 73 | 1-121:2-154 | 1:1 | 2.87 | 142.8 | Green | 523 |
| Example 74 | | 1:2 | 2.91 | 141.3 | Green | 534 |
| Example 75 | | 1:3 | 2.93 | 140.6 | Green | 549 |
| Example 76 | 1-125:2-154 | 1:1 | 2.11 | 159.3 | Green | 601 |
| Example 77 | | 1:2 | 2.19 | 158.4 | Green | 613 |
| Example 78 | | 1:3 | 2.23 | 157.2 | Green | 623 |
| Example 79 | 1-126:2-158 | 1:1 | 2.03 | 158.6 | Green | 586 |
| Example 80 | | 1:2 | 2.14 | 157.2 | Green | 593 |
| Example 81 | | 1:3 | 2.26 | 156.3 | Green | 602 |
| Example 82 | 1-129:2-154 | 1:1 | 1.38 | 157.2 | Green | 578 |
| Example 83 | | 1:2 | 1.43 | 156.3 | Green | 588 |
| Example 84 | | 1:3 | 1.52 | 155.2 | Green | 593 |
| Example 85 | 1-132:2-158 | 1:1 | 1.44 | 158.4 | Green | 563 |
| Example 86 | | 1:2 | 1.52 | 157.3 | Green | 572 |
| Example 87 | | 1:3 | 1.58 | 156.1 | Green | 588 |
| Example 88 | 1-141:2-158 | 1:1 | 1.32 | 157.7 | Green | 566 |
| Example 89 | | 1:2 | 1.36 | 157.2 | Green | 572 |
| Example 90 | | 1:3 | 1.47 | 156.0 | Green | 583 |
| Example 91 | 1-143:2-149 | 1:1 | 2.03 | 157.3 | Green | 550 |
| Example 92 | | 1:2 | 2.13 | 156.8 | Green | 556 |
| Example 93 | | 1:3 | 2.26 | 156.1 | Green | 568 |
| Example 94 | 1-145:2-146 | 1:1 | 2.14 | 155.3 | Green | 562 |
| Example 95 | | 1:2 | 2.26 | 154.8 | Green | 573 |
| Example 96 | | 1:3 | 2.33 | 153.2 | Green | 582 |
| Example 97 | 1-150:2-154 | 1:1 | 2.18 | 158.5 | Green | 568 |
| Example 98 | | 1:2 | 2.23 | 157.3 | Green | 573 |
| Example 99 | | 1:3 | 2.35 | 157.2 | Green | 582 |
| Example 100 | 1-154:2-154 | 1:1 | 1.69 | 157.3 | Green | 593 |
| Example 101 | | 1:2 | 1.73 | 156.5 | Green | 602 |
| Example 102 | | 1:3 | 1.77 | 155.3 | Green | 613 |
| Example 103 | 1-158:2-149 | 1:1 | 1.05 | 157.3 | Green | 568 |
| Example 104 | | 1:2 | 1.13 | 156.5 | Green | 572 |
| Example 105 | | 1:3 | 1.23 | 155.3 | Green | 589 |
| Example 106 | 1-173:2-158 | 1:1 | 1.26 | 159.3 | Green | 563 |
| Example 107 | | 1:2 | 1.36 | 158.4 | Green | 575 |
| Example 108 | | 1:3 | 1.44 | 157.2 | Green | 583 |
| Example 109 | 1-177:2-154 | 1:1 | 1.23 | 157.3 | Green | 556 |
| Example 110 | | 1:2 | 1.32 | 156.1 | Green | 563 |
| Example 111 | | 1:3 | 1.41 | 155.5 | Green | 572 |
| Example 112 | 1-182:2-146 | 1:1 | 1.63 | 160.2 | Green | 561 |
| Example 113 | | 1:2 | 1.69 | 159.4 | Green | 579 |
| Example 114 | | 1:3 | 1.72 | 158.2 | Green | 583 |
| Example 115 | 1-185:2-146 | 1:1 | 1.65 | 153.2 | Green | 558 |
| Example 116 | | 1:2 | 1.72 | 152.4 | Green | 563 |
| Example 117 | | 1:3 | 1.83 | 151.6 | Green | 572 |
| Example 118 | 1-189:2-154 | 1:1 | 1.52 | 149.5 | Green | 559 |
| Example 119 | | 1:2 | 1.56 | 148.4 | Green | 563 |
| Example 120 | | 1:3 | 1.73 | 147.3 | Green | 573 |
| Example 121 | 1-199:2-154 | 1:1 | 1.66 | 148.5 | Green | 549 |
| Example 122 | | 1:2 | 1.72 | 148.3 | Green | 553 |
| Example 123 | | 1:3 | 1.77 | 147.5 | Green | 567 |
| Example 124 | 1-201:2-154 | 1:1 | 1.52 | 143.2 | Green | 573 |
| Example 125 | | 1:2 | 1.53 | 142.6 | Green | 585 |
| Example 126 | | 1:3 | 1.56 | 141.3 | Green | 593 |
| Example 127 | 1-203:2-158 | 1:1 | 1.63 | 145.2 | Green | 612 |
| Example 128 | | 1:2 | 1.65 | 143.7 | Green | 622 |
| Example 129 | | 1:3 | 1.72 | 142.2 | Green | 638 |
| Example 130 | 1-212:2-146 | 1:1 | 1.32 | 146.3 | Green | 605 |
| Example 131 | | 1:2 | 1.42 | 145.8 | Green | 618 |
| Example 132 | | 1:3 | 1.53 | 144.3 | Green | 628 |
| Example 133 | 1-214:2-146 | 1:1 | 1.46 | 145.8 | Green | 612 |
| Example 134 | | 1:2 | 1.52 | 144.2 | Green | 623 |
| Example 135 | | 1:3 | 1.63 | 143.6 | Green | 638 |
| Example 136 | 1-218:2-149 | 1:1 | 1.44 | 146.2 | Green | 628 |
| Example 137 | | 1:2 | 1.53 | 145.6 | Green | 633 |
| Example 138 | | 1:3 | 1.68 | 144.8 | Green | 649 |
| Example 139 | 1-221:2-149 | 1:1 | 1.23 | 143.8 | Green | 583 |
| Example 140 | | 1:2 | 1.33 | 142.6 | Green | 592 |
| Example 141 | | 1:3 | 1.42 | 141.3 | Green | 604 |
| Example 142 | 1-247:2-158 | 1:1 | 2.13 | 155.6 | Green | 583 |
| Example 143 | | 1:2 | 2.04 | 153.2 | Green | 594 |
| Example 144 | | 1:3 | 1.98 | 151.8 | Green | 601 |
| Example 145 | 1-249:2-154 | 1:1 | 3.23 | 98.7 | Green | 302 |
| Example 146 | | 1:2 | 3.35 | 98.0 | Green | 312 |
| Example 147 | | 1:3 | 3.42 | 97.6 | Green | 322 |
| Example 148 | 1-253:2-154 | 1:1 | 3.15 | 97.3 | Green | 315 |
| Example 149 | | 1:2 | 3.28 | 96.5 | Green | 326 |
| Example 150 | | 1:3 | 3.38 | 95.4 | Green | 338 |
| Example 151 | 1-257:2-158 | 1:1 | 4.02 | 99.3 | Green | 283 |
| Example 152 | | 1:2 | 4.15 | 98.5 | Green | 297 |
| Example 153 | | 1:3 | 4.22 | 97.1 | Green | 306 |
| Example 154 | 1-261:2-158 | 1:1 | 4.13 | 100.5 | Green | 299 |
| Example 155 | | 1:2 | 4.26 | 99.3 | Green | 308 |
| Example 156 | | 1:3 | 4.38 | 98.4 | Green | 312 |
| Example 157 | 1-263:2-149 | 1:1 | 3.78 | 106.5 | Green | 322 |
| Example 158 | | 1:2 | 3.87 | 101.3 | Green | 336 |
| Example 159 | | 1:3 | 3.91 | 100.7 | Green | 342 |
| Example 160 | 1-265:2-154 | 1:1 | 3.44 | 103.5 | Green | 305 |
| Example 161 | | 1:2 | 3.58 | 102.2 | Green | 312 |
| Example 162 | | 1:3 | 3.62 | 101.3 | Green | 326 |
| Example 163 | 1-275:2-154 | 1:1 | 4.12 | 108.7 | Green | 287 |
| Example 164 | | 1:2 | 4.23 | 105.5 | Green | 293 |
| Example 165 | | 1:3 | 4.36 | 104.6 | Green | 300 |
| Example 166 | 1-279:2-146 | 1:1 | 4.22 | 97.6 | Green | 267 |
| Example 167 | | 1:2 | 4.37 | 96.5 | Green | 279 |
| Example 168 | | 1:3 | 4.45 | 95.3 | Green | 283 |
| Example 169 | 1-282:2-158 | 1:1 | 3.62 | 98.7 | Green | 256 |
| Example 170 | | 1:2 | 3.71 | 97.2 | Green | 266 |
| Example 171 | | 1:3 | 3.83 | 96.5 | Green | 275 |
| Example 172 | 1-287:2-154 | 1:1 | 3.56 | 97.7 | Green | 268 |
| Example 173 | | 1:2 | 3.62 | 97.5 | Green | 274 |
| Example 174 | | 1:3 | 3.72 | 96.3 | Green | 285 |
| Example 175 | 1-290:2-149 | 1:1 | 3.46 | 98.5 | Green | 268 |
| Example 176 | | 1:2 | 3.52 | 97.3 | Green | 275 |
| Example 177 | | 1:3 | 3.63 | 96.1 | Green | 283 |
| Example 178 | 1-294:2-154 | 1:1 | 3.15 | 110.2 | Green | 253 |
| Example 179 | | 1:2 | 3.28 | 109.5 | Green | 269 |
| Example 180 | | 1:3 | 3.36 | 108.7 | Green | 276 |
| Example 181 | 1-298:2-158 | 1:1 | 3.24 | 86.7 | Green | 268 |
| Example 182 | | 1:2 | 3.35 | 86.2 | Green | 277 |
| Example 183 | | 1:3 | 3.44 | 85.2 | Green | 283 |
| Example 184 | 1-302:2-149 | 1:1 | 3.04 | 90.5 | Green | 302 |
| Example 185 | | 1:2 | 3.12 | 89.3 | Green | 312 |
| Example 186 | | 1:3 | 3.28 | 88.2 | Green | 326 |
| Example 187 | 1-309:2-146 | 1:1 | 3.15 | 93.2 | Green | 312 |
| Example 188 | | 1:2 | 3.26 | 92.1 | Green | 332 |
| Example 189 | | 1:3 | 3.38 | 91.3 | Green | 342 |
| Example 190 | 1-310:2-146 | 1:1 | 3.23 | 95.4 | Green | 328 |
| Example 191 | | 1:2 | 3.36 | 94.3 | Green | 337 |
| Example 192 | | 1:3 | 3.42 | 93.2 | Green | 349 |
| Example 193 | 1-313:2-154 | 1:1 | 4.23 | 96.5 | Green | 302 |
| Example 194 | | 1:2 | 4.36 | 95.2 | Green | 313 |
| Example 195 | | 1:3 | 4.48 | 94.7 | Green | 326 |
| Example 196 | 1-323:2-154 | 1:1 | 3.77 | 97.4 | Green | 268 |
| Example 197 | | 1:2 | 3.82 | 96.3 | Green | 272 |
| Example 198 | | 1:3 | 3.93 | 95.2 | Green | 285 |
| Example 199 | 1-324:2-146 | 1:1 | 4.03 | 87.3 | Green | 279 |
| Example 200 | | 1:2 | 4.13 | 86.5 | Green | 283 |
| Example 201 | | 1:3 | 4.25 | 85.2 | Green | 291 |
| Example 202 | 1-327:2-154 | 1:1 | 4.23 | 93.4 | Green | 276 |
| Example 203 | | 1:2 | 4.38 | 92.1 | Green | 283 |
| Example 204 | | 1:3 | 4.50 | 91.3 | Green | 285 |
| Example 205 | 1-330:2-158 | 1:1 | 3.25 | 88.3 | Green | 277 |
| Example 206 | | 1:2 | 3.30 | 87.2 | Green | 281 |
| Example 207 | | 1:3 | 3.32 | 86.6 | Green | 299 |
| Example 208 | 1-335:2-158 | 1:1 | 3.25 | 87.5 | Green | 263 |
| Example 209 | | 1:2 | 3.34 | 86.3 | Green | 275 |
| Example 210 | | 1:3 | 3.59 | 85.5 | Green | 281 |
| Example 211 | 1-339:2-149 | 1:1 | 4.23 | 93.2 | Green | 266 |
| Example 212 | | 1:2 | 4.32 | 92.3 | Green | 273 |
| Example 213 | | 1:3 | 4.44 | 91.5 | Green | 276 |
| Example 214 | 1-348:2-122 | 1:1 | 4.21 | 98.6 | Green | 266 |
| Example 215 | | 1:2 | 4.36 | 97.4 | Green | 271 |
| Example 216 | | 1:3 | 4.42 | 96.1 | Green | 283 |
| Example 217 | 1-348:2-154 | 1:1 | 3.26 | 110.3 | Green | 323 |
| Example 218 | | 1:2 | 3.34 | 101.6 | Green | 338 |
| Example 219 | | 1:3 | 3.48 | 99.3 | Green | 342 |
| Example 220 | 1-350:2-154 | 1:1 | 3.23 | 134.5 | Green | 402 |
| Example 221 | | 1:2 | 3.20 | 133.4 | Green | 413 |
| Example 222 | | 1:3 | 3.16 | 132.6 | Green | 426 |
| Example 223 | 1-358:2-146 | 1:1 | 3.48 | 132.7 | Green | 423 |
| Example 224 | | 1:2 | 3.33 | 131.5 | Green | 438 |
| Example 225 | | 1:3 | 3.27 | 130.3 | Green | 442 |
| Example 226 | 1-360:2-154 | 1:1 | 3.98 | 135.1 | Green | 452 |
| Example 227 | | 1:2 | 3.84 | 134.6 | Green | 463 |
| Example 228 | | 1:3 | 3.82 | 133.2 | Green | 466 |
| Example 229 | 1-366:2-146 | 1:1 | 3.74 | 130.4 | Green | 437 |
| Example 230 | | 1:2 | 3.62 | 129.5 | Green | 442 |
| Example 231 | | 1:3 | 3.53 | 128.7 | Green | 456 |
| Example 232 | 1-369:2-149 | 1:1 | 3.77 | 132.5 | Green | 463 |
| Example 233 | | 1:2 | 3.65 | 131.1 | Green | 471 |
| Example 234 | | 1:3 | 3.42 | 130.6 | Green | 475 |
| Example 235 | 1-375:2-146 | 1:1 | 4.23 | 129.3 | Green | 387 |
| Example 236 | | 1:2 | 4.11 | 128.7 | Green | 392 |
| Example 237 | | 1:3 | 4.05 | 127.7 | Green | 402 |
| Example 238 | 1-383:2-154 | 1:1 | 4.35 | 132.6 | Green | 413 |
| Example 239 | | 1:2 | 4.28 | 131.5 | Green | 423 |
| Example 240 | | 1:3 | 4.13 | 130.2 | Green | 448 |
| Example 241 | 1-388:2-158 | 1:1 | 4.52 | 133.7 | Green | 432 |
| Example 242 | | 1:2 | 4.43 | 132.5 | Green | 445 |
| Example 243 | | 1:3 | 4.38 | 131.4 | Green | 467 |
| Example 244 | 1-389:2-146 | 1:1 | 4.41 | 130.5 | Green | 359 |
| Example 245 | | 1:2 | 4.36 | 129.7 | Green | 350 |
| Example 246 | | 1:3 | 4.25 | 128.5 | Green | 341 |
| Example 247 | 1-392:2-154 | 1:1 | 4.37 | 133.5 | Green | 415 |
| Example 248 | | 1:2 | 4.35 | 132.7 | Green | 402 |
| Example 249 | | 1:3 | 4.26 | 131.3 | Green | 399 |
| Example 250 | 1-397:2-146 | 1:1 | 4.33 | 134.4 | Green | 423 |
| Example 251 | | 1:2 | 4.26 | 133.2 | Green | 415 |
| Example 252 | | 1:3 | 4.18 | 132.6 | Green | 394 |
| Example 253 | 1-399:2-154 | 1:1 | 3.68 | 134.7 | Green | 413 |
| Example 254 | | 1:2 | 3.72 | 133.5 | Green | 425 |
| Example 255 | | 1:3 | 3.88 | 132.8 | Green | 438 |
| Example 256 | 1-404:2-158 | 1:1 | 3.59 | 135.2 | Green | 426 |
| Example 257 | | 1:2 | 3.63 | 134.5 | Green | 438 |
| Example 258 | | 1:3 | 3.72 | 133.7 | Green | 442 |
| Example 259 | 1-410:2-154 | 1:1 | 4.23 | 115.3 | Green | 436 |
| Example 260 | | 1:2 | 4.38 | 114.2 | Green | 442 |
| Example 261 | | 1:3 | 4.45 | 113.8 | Green | 456 |
| Example 262 | 1-413:2-154 | 1:1 | 4.26 | 111.6 | Green | 462 |
| Example 263 | | 1:2 | 4.38 | 110.3 | Green | 473 |
| Example 264 | | 1:3 | 4.42 | 110.2 | Green | 485 |
| Example 265 | 1-418:2-158 | 1:1 | 4.23 | 123.5 | Green | 361 |
| Example 266 | | 1:2 | 4.38 | 122.4 | Green | 375 |
| Example 267 | | 1:3 | 4.42 | 121.2 | Green | 383 |
| Example 268 | 1-421:2-158 | 1:1 | 4.32 | 124.3 | Green | 358 |
| Example 269 | | 1:2 | 4.35 | 123.5 | Green | 366 |
| Example 270 | | 1:3 | 4.52 | 122.0 | Green | 372 |
| Example 271 | 1-427:2-149 | 1:1 | 4.12 | 119.8 | Green | 364 |
| Example 272 | | 1:2 | 4.32 | 117.3 | Green | 371 |
| Example 273 | | 1:3 | 4.38 | 116.5 | Green | 382 |
| Example 274 | 1-430:2-146 | 1:1 | 4.22 | 118.7 | Green | 378 |
| Example 275 | | 1:2 | 4.36 | 117.5 | Green | 388 |
| Example 276 | | 1:3 | 4.48 | 116.1 | Green | 391 |
| Example 277 | 1-436:2-154 | 1:1 | 4.32 | 125.2 | Green | 387 |
| Example 278 | | 1:2 | 4.35 | 124.3 | Green | 392 |
| Example 279 | | 1:3 | 4.44 | 123.7 | Green | 412 |
| Example 280 | 1-441:2-154 | 1:1 | 4.13 | 124.6 | Green | 405 |
| Example 281 | | 1:2 | 4.28 | 123.5 | Green | 413 |
| Example 282 | | 1:3 | 4.36 | 120.2 | Green | 426 |
| Example 283 | 1-445:2-158 | 1:1 | 4.22 | 125.9 | Green | 415 |
| Example 284 | | 1:2 | 4.38 | 123.7 | Green | 427 |
| Example 285 | | 1:3 | 4.45 | 121.4 | Green | 436 |
| Example 286 | 1-450:2-158 | 1:1 | 4.05 | 124.8 | Green | 426 |
| Example 287 | | 1:2 | 4.13 | 123.5 | Green | 438 |
| Example 288 | | 1:3 | 4.29 | 122.6 | Green | 444 |
| Example 289 | 1-452:2-154 | 1:1 | 4.23 | 125.9 | Green | 457 |
| Example 290 | | 1:2 | 4.32 | 124.7 | Green | 460 |
| Example 291 | | 1:3 | 4.41 | 123.2 | Green | 472 |
| Example 292 | 1-469:2-130 | 1:1 | 4.42 | 126.5 | Green | 342 |
| Example 293 | | 1:2 | 4.49 | 125.1 | Green | 353 |
| Example 294 | | 1:3 | 4.53 | 124.8 | Green | 361 |
| Example 295 | 1-469:2-149 | 1:1 | 3.71 | 134.2 | Green | 452 |
| Example 296 | | 1:2 | 3.78 | 133.6 | Green | 466 |
| Example 297 | | 1:3 | 3.84 | 132.7 | Green | 470 |

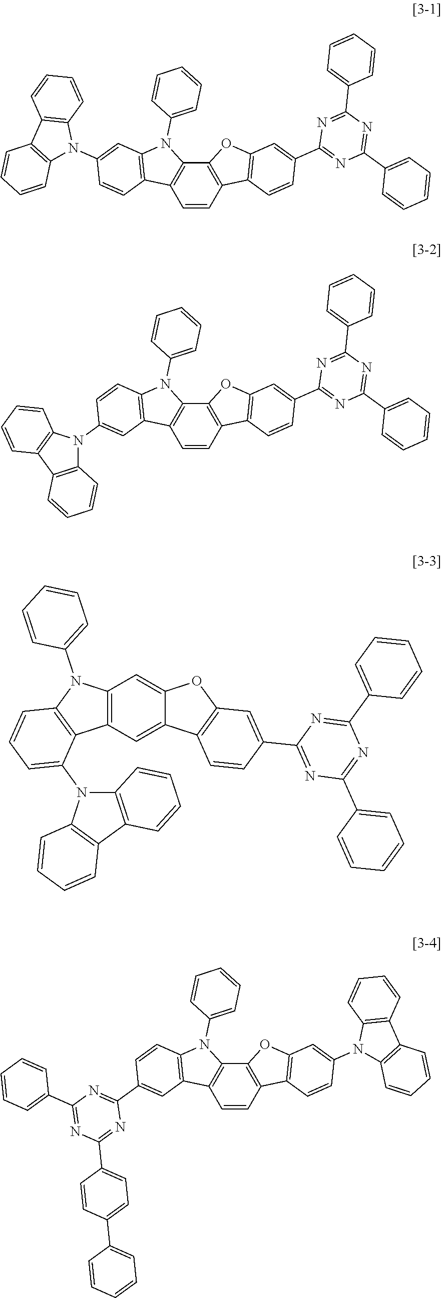 [SMILES: c1ccc(-c2nc(-c3ccccc3)nc(-c3ccc4c(c3)oc3c4ccc4c5ccc(-n6c7ccccc7c7ccccc76)cc5n(-c5ccccc5)c43)n2)cc1 ; c1ccc(-c2nc(-c3ccccc3)nc(-c3ccc4c(c3)oc3c4ccc4c5cc(-n6c7ccccc7c7ccccc76)ccc5n(-c5ccccc5)c43)n2)cc1 ; c1ccc(-c2nc(-c3ccccc3)nc(-c3ccc4c(c3)oc3cc5c(cc34)c3c(-n4c6ccccc6c6ccccc64)cccc3n5-c3ccccc3)n2)cc1 ; c1ccc(-c2ccc(-c3nc(-c4ccccc4)nc(-c4ccc5c(c4)c4ccc6c7ccc(-n8c9ccccc9c9ccccc98)cc7oc6c4n5-c4ccccc4)n3)cc2)cc1]

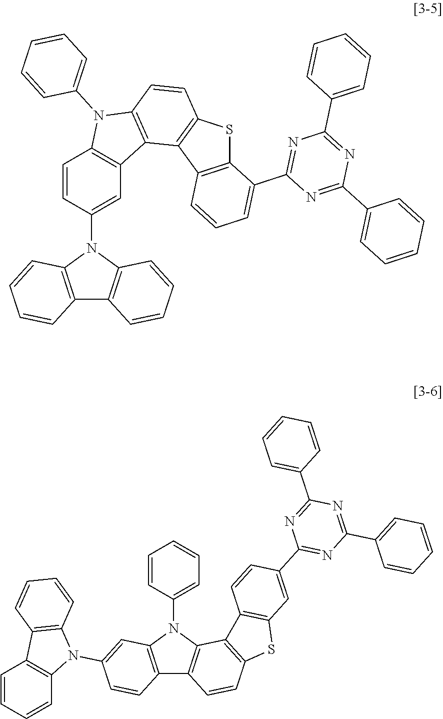 [SMILES: c1ccc(-c2nc(-c3ccccc3)nc(-c3cccc4c3sc3ccc5c(c6cc(-n7c8ccccc8c8ccccc87)ccc6n5-c5ccccc5)c34)n2)cc1 ; c1ccc(-c2nc(-c3ccccc3)nc(-c3ccc4c(c3)sc3ccc5c6ccc(-n7c8ccccc8c8ccccc87)cc6n(-c6ccccc6)c5c34)n2)cc1]

TABLE 10

| | | Driving | | | Service |
|---|---|---|---|---|---|
| | | voltage | efficiency | Color | life |
| | Compound | (V) | (cd/A) | coordinate | (T₉₀) |
| Comparative | 3-1 | 6.23 | 40.8 | Green | 75 |
| Example 19 | | | | | |
| Comparative | 3-2 | 6.31 | 43.9 | Green | 73 |
| Example 20 | | | | | |
| Comparative | 3-3 | 6.12 | 44.7 | Green | 68 |
| Example 21 | | | | | |
| Comparative | 3-4 | 6.08 | 42.5 | Green | 71 |
| Example 22 | | | | | |
| Comparative | 3-5 | 6.11 | 41.7 | Green | 63 |
| Example 23 | | | | | |
| Comparative | 3-6 | 6.49 | 43.2 | Green | 65 |
| Example 24 | | | | | |
| Example 298 | 1-1 | 3.87 | 103.8 | Green | 330 |
| Example 299 | 1-5 | 3.02 | 110.3 | Green | 335 |
| Example 300 | 1-12 | 4.38 | 108.7 | Green | 413 |
| Example 301 | 1-22 | 4.12 | 120.3 | Green | 422 |
| Example 302 | 1-32 | 3.98 | 116.5 | Green | 432 |
| Example 303 | 1-38 | 3.87 | 103.7 | Green | 440 |
| Example 304 | 1-42 | 4.11 | 102.6 | Green | 387 |
| Example 305 | 1-46 | 4.32 | 120.3 | Green | 394 |
| Example 306 | 1-50 | 3.56 | 111.9 | Green | 381 |
| Example 307 | 1-53 | 3.82 | 119.3 | Green | 374 |
| Example 308 | 1-60 | 3.92 | 112.1 | Green | 385 |
| Example 309 | 1-63 | 3.68 | 110.3 | Green | 391 |
| Example 310 | 1-65 | 4.23 | 100.9 | Green | 385 |
| Example 311 | 1-72 | 4.50 | 113.7 | Green | 384 |
| Example 312 | 1-79 | 4.12 | 115.3 | Green | 332 |
| Example 313 | 1-81 | 4.03 | 117.3 | Green | 342 |
| Example 314 | 1-88 | 3.77 | 106.7 | Green | 351 |
| Example 315 | 1-91 | 3.89 | 108.3 | Green | 364 |
| Example 316 | 1-97 | 4.11 | 105.8 | Green | 379 |
| Example 317 | 1-100 | 4.32 | 111.6 | Green | 382 |
| Example 318 | 1-102 | 4.23 | 100.3 | Green | 388 |
| Example 319 | 1-108 | 3.68 | 120.3 | Green | 394 |
| Example 320 | 1-113 | 4.12 | 111.8 | Green | 440 |
| Example 321 | 1-120 | 3.50 | 103.8 | Green | 398 |
| Example 322 | 1-121 | 3.51 | 119.4 | Green | 442 |
| Example 323 | 1-125 | 3.42 | 121.6 | Green | 447 |
| Example 324 | 1-126 | 3.33 | 130.2 | Green | 452 |
| Example 325 | 1-129 | 3.09 | 128.4 | Green | 487 |
| Example 326 | 1-132 | 3.12 | 110.6 | Green | 469 |
| Example 327 | 1-141 | 2.98 | 113.7 | Green | 472 |
| Example 328 | 1-143 | 3.28 | 115.3 | Green | 488 |
| Example 329 | 1-145 | 3.12 | 123.7 | Green | 520 |
| Example 330 | 1-150 | 2.73 | 121.6 | Green | 429 |
| Example 331 | 1-154 | 2.69 | 130.9 | Green | 415 |
| Example 332 | 1-158 | 2.56 | 112.7 | Green | 406 |
| Example 333 | 1-173 | 2.74 | 110.3 | Green | 441 |
| Example 334 | 1-177 | 2.87 | 126.5 | Green | 452 |
| Example 335 | 1-182 | 2.98 | 125.1 | Green | 468 |
| Example 336 | 1-185 | 2.67 | 124.8 | Green | 527 |
| Example 337 | 1-189 | 2.59 | 123.4 | Green | 536 |
| Example 338 | 1-199 | 2.33 | 122.6 | Green | 527 |
| Example 339 | 1-201 | 3.18 | 129.7 | Green | 506 |
| Example 340 | 1-203 | 3.26 | 128.5 | Green | 513 |
| Example 341 | 1-212 | 3.02 | 127.2 | Green | 529 |
| Example 342 | 1-214 | 3.44 | 121.6 | Green | 532 |
| Example 343 | 1-218 | 3.26 | 123.4 | Green | 412 |
| Example 344 | 1-221 | 3.12 | 122.7 | Green | 403 |
| Example 345 | 1-247 | 2.50 | 129.7 | Green | 540 |
| Example 346 | 1-249 | 4.63 | 75.4 | Green | 159 |
| Example 347 | 1-253 | 4.76 | 74.6 | Green | 162 |
| Example 348 | 1-257 | 4.87 | 73.2 | Green | 160 |
| Example 349 | 1-261 | 5.62 | 71.9 | Green | 200 |
| Example 350 | 1-263 | 5.44 | 70.3 | Green | 187 |
| Example 351 | 1-265 | 5.46 | 68.7 | Green | 193 |
| Example 352 | 1-275 | 5.32 | 65.1 | Green | 188 |
| Example 353 | 1-279 | 5.44 | 61.2 | Green | 153 |
| Example 354 | 1-282 | 5.50 | 63.4 | Green | 162 |
| Example 355 | 1-287 | 5.42 | 64.8 | Green | 172 |
| Example 356 | 1-290 | 5.39 | 65.8 | Green | 177 |
| Example 357 | 1-294 | 5.26 | 64.2 | Green | 182 |
| Example 358 | 1-298 | 5.13 | 60.5 | Green | 188 |
| Example 359 | 1-302 | 4.59 | 73.4 | Green | 193 |
| Example 360 | 1-309 | 4.72 | 74.8 | Green | 197 |
| Example 361 | 1-310 | 4.63 | 75.2 | Green | 182 |
| Example 362 | 1-313 | 4.56 | 74.6 | Green | 180 |
| Example 363 | 1-323 | 4.52 | 93.4 | Green | 285 |
| Example 364 | 1-324 | 5.13 | 99.2 | Green | 264 |
| Example 365 | 1-327 | 5.23 | 100.3 | Green | 259 |
| Example 366 | 1-330 | 5.55 | 98.4 | Green | 245 |
| Example 367 | 1-335 | 5.47 | 97.6 | Green | 234 |
| Example 368 | 1-339 | 5.32 | 83.4 | Green | 223 |
| Example 369 | 1-348 | 4.52 | 75.4 | Green | 201 |
| Example 370 | 1-350 | 5.26 | 88.5 | Green | 213 |
| Example 371 | 1-358 | 5.18 | 87.6 | Green | 200 |
| Example 372 | 1-360 | 5.20 | 80.4 | Green | 219 |
| Example 373 | 1-366 | 4.68 | 82.6 | Green | 321 |
| Example 374 | 1-369 | 4.78 | 81.4 | Green | 316 |
| Example 375 | 1-375 | 4.82 | 83.6 | Green | 324 |
| Example 376 | 1-383 | 5.13 | 84.7 | Green | 312 |
| Example 377 | 1-388 | 5.26 | 90.4 | Green | 302 |
| Example 378 | 1-389 | 5.37 | 91.6 | Green | 263 |
| Example 379 | 1-392 | 5.46 | 92.4 | Green | 255 |
| Example 380 | 1-397 | 4.98 | 93.8 | Green | 241 |
| Example 381 | 1-399 | 4.87 | 94.7 | Green | 237 |
| Example 382 | 1-404 | 4.69 | 92.4 | Green | 226 |
| Example 383 | 1-410 | 4.68 | 93.8 | Green | 213 |
| Example 384 | 1-413 | 5.23 | 94.8 | Green | 206 |
| Example 385 | 1-418 | 5.38 | 99.4 | Green | 265 |
| Example 386 | 1-421 | 5.52 | 80.7 | Green | 287 |
| Example 387 | 1-427 | 5.47 | 93.5 | Green | 298 |
| Example 388 | 1-430 | 5.23 | 87.6 | Green | 305 |
| Example 389 | 1-436 | 4.89 | 80.4 | Green | 315 |
| Example 390 | 1-441 | 4.77 | 83.7 | Green | 320 |
| Example 391 | 1-445 | 4.69 | 97.4 | Green | 253 |
| Example 392 | 1-450 | 4.53 | 98.5 | Green | 241 |
| Example 393 | 1-452 | 4.72 | 99.4 | Green | 233 |
| Example 394 | 1-469 | 4.52 | 100.3 | Green | 312 |

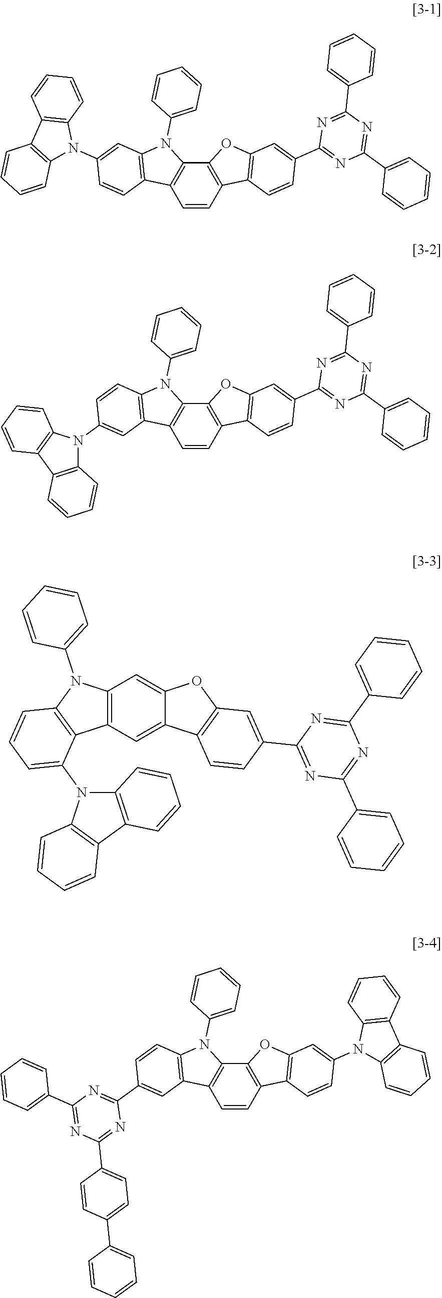 [SMILES: c1ccc(-c2nc(-c3ccccc3)nc(-c3ccc4c(c3)oc3c4ccc4c5ccc(-n6c7ccccc7c7ccccc76)cc5n(-c5ccccc5)c43)n2)cc1 ; c1ccc(-c2nc(-c3ccccc3)nc(-c3ccc4c(c3)oc3c4ccc4c5cc(-n6c7ccccc7c7ccccc76)ccc5n(-c5ccccc5)c43)n2)cc1 ; c1ccc(-c2nc(-c3ccccc3)nc(-c3ccc4c(c3)oc3cc5c(cc34)c3c(-n4c6ccccc6c6ccccc64)cccc3n5-c3ccccc3)n2)cc1 ; c1ccc(-c2ccc(-c3nc(-c4ccccc4)nc(-c4ccc5c(c4)c4ccc6c7ccc(-n8c9ccccc9c9ccccc98)cc7oc6c4n5-c4ccccc4)n3)cc2)cc1]

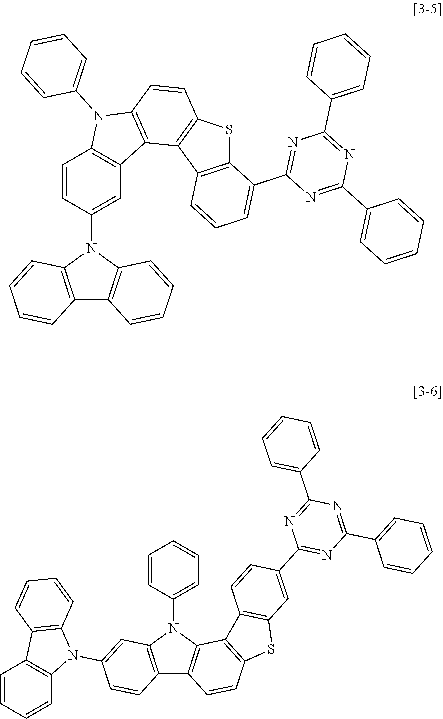 [SMILES: c1ccc(-c2nc(-c3ccccc3)nc(-c3cccc4c3sc3ccc5c(c6cc(-n7c8ccccc8c8ccccc87)ccc6n5-c5ccccc5)c34)n2)cc1 ; c1ccc(-c2nc(-c3ccccc3)nc(-c3ccc4c(c3)sc3ccc5c6ccc(-n7c8ccccc8c8ccccc87)cc6n(-c6ccccc6)c5c34)n2)cc1]

Referring to the results of Table 9 and Table 10, it can be seen that an organic light emitting device including the heterocyclic compound of the present invention has excellent driving voltage, light emitting efficiency and service life than the Comparative Examples. In particular, it can be confirmed that the higher the deuterium substitution rate, the lower the driving voltage and the better the service life characteristics.

Specifically, the heterocyclic compound represented by Chemical Formula 1 according to the present application is a compound which has benzofurocarbazole or benzothienocarbazole having a structure in which X is O; or S as a core, and since the heterocyclic compound forms a resonance structure, the substituents of R1, R5, R6 and R8 become relatively electron-rich, and thus have negative charges. In the compound, this site is substituted with Chemical Formula A or B having an unshared pair of electrons, and the HOMO value is increased because the hole characteristics of the molecule are enhanced.

Accordingly, since hole traps do not occur in adjacent layers of an OLED including the heterocyclic compound of Chemical Formula 1, and holes are smoothly transferred, it could be confirmed that it is characterized by improving the driving and service life of the OLED compared to OLEDs including the compounds of the Comparative Examples.

In Tables 9 and 10, the compounds of the Comparative Examples correspond to cases where at least one of R1, R5, R6 and R8 in Chemical Formula 1 of the present invention is not represented by Chemical Formula A or B.

That is, Compounds 3-1 and 3-6, Compounds 3-2 and 3-5, Compound 3-3, and Compound 3-4 used as Comparative Examples correspond to cases where the compound of Chemical Formula A or B is substituted at the R2 position, at the R3 position, at the R4 position, and at the R7 position, respectively. In this case, since hole traps occurred in adjacent layers, holes were not smoothly transferred, so that it could be confirmed that driving and efficiency were not as good as those of the OLED used in the Examples, and the service life was also shortened.

In addition, when comparing the results in Table 10 with the results in Table 9, it can be confirmed that when both the heterocyclic compound of Chemical Formula 1 and the compound of Chemical Formula 2 are used as the light emitting layer host, the service life is improved by about three times, and the driving voltage and the light emitting efficiency are improved about 40% and about 50%, respectively.

In contrast, it can be seen that when a compound not included in the scope of the present invention is used in combination with the compound of Chemical Formula 2 (Comparative Examples 1 to 18 of Table 9), the service life is similar to that when the heterocyclic compound of the present invention is used alone, and the performance deteriorates in terms of driving voltage and light emitting efficiency.

That is, it could be confirmed that when both the heterocyclic compound of Chemical Formula 1 and the compound of Chemical Formula 2 of the present invention are used as hosts of a light emitting layer, the driving voltage, light emitting efficiency and service life are remarkably excellent.

1. A heterocyclic compound represented by the following Chemical Formula 1: 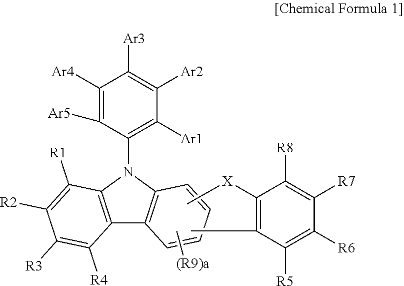
wherein, in Chemical Formula 1,
X is O; or S,
Ar1 to Ar5 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group, or adjacent groups are bonded to each other to form a substituted or unsubstituted C6 to C60 aromatic hydrocarbon ring; or a substituted or unsubstituted C2 to C60 aromatic hetero ring,
R1 to R9 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′,
a is an integer from 0 to 2, and when a is 2, substituents in the parenthesis are the same as or different from each other,
at least one of R1, R5, R6 and R8 is represented by the following Chemical Formula A or B, 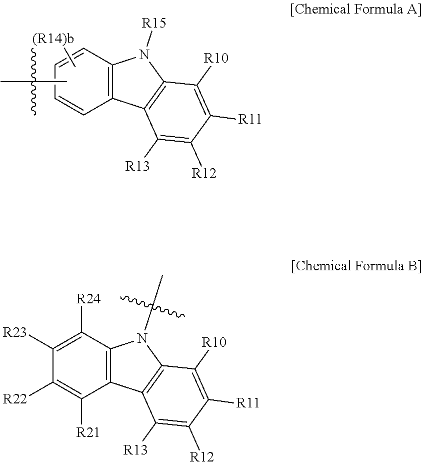
in Chemical Formulae A and B,
R10 to R15 and R21 to R24 are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′,
b is an integer from 0 to 3, and when b is 2 or higher, substituents in the parenthesis are the same as or different from each other,
in R1 to R8, at least one of the substituents other than the substituent represented by Chemical Formula A or B is a group represented by —N-Het, and the —N-Het is a substituted or unsubstituted C2 to C60 heteroaryl group including N, and
R, R′ and R″ are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group.

2. The heterocyclic compound of claim 1, wherein Chemical Formula 1 is represented by any one of the following Chemical Formulae 3 to 8: 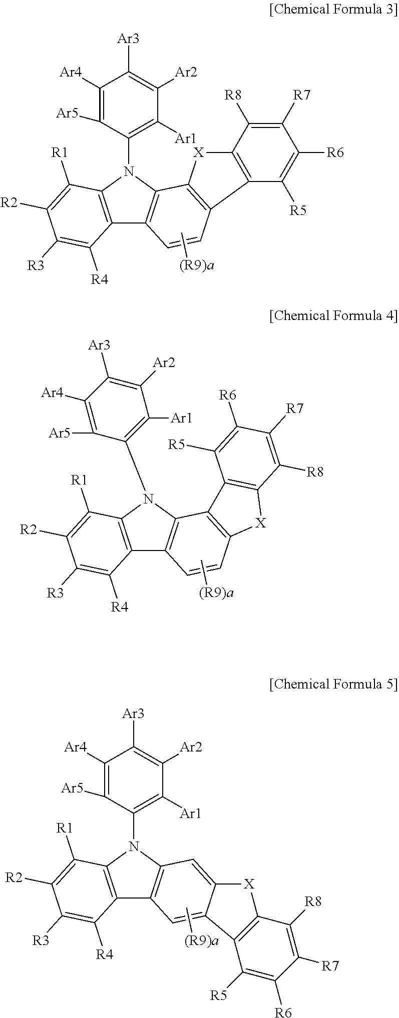 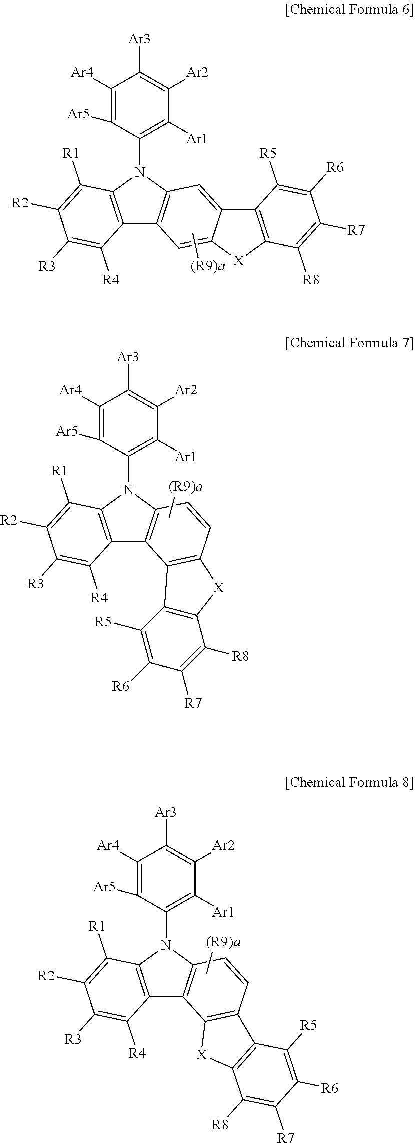
in Chemical Formulae 3 to 8,
the definition of each substituent is the same as the definition in Chemical Formula 1.

3. The heterocyclic compound of claim 1, wherein Chemical Formula B is represented by any one of the following Chemical Formulae B-1 to B-5: 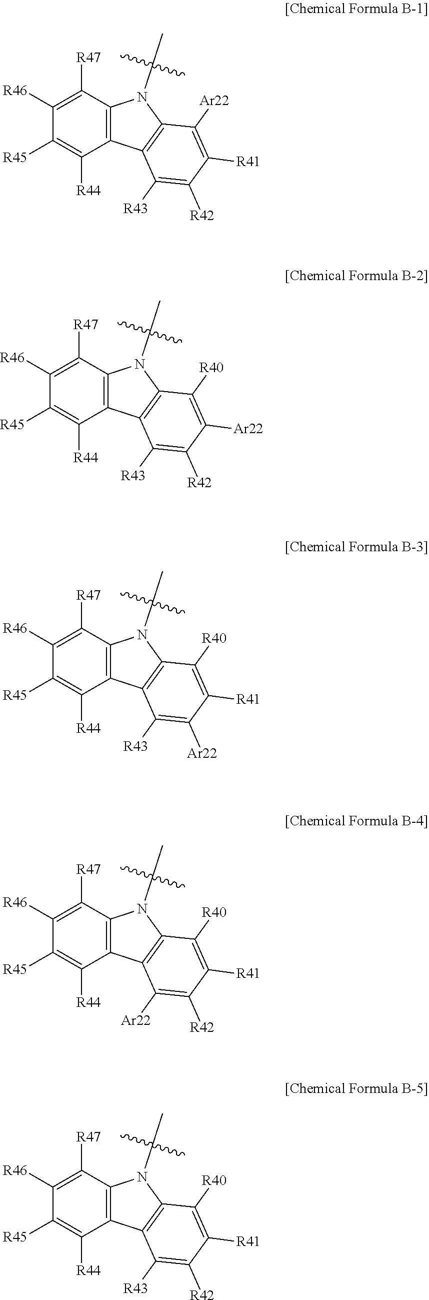
in Chemical Formulae B-1 to B-5,
Ar22 is a substituted or unsubstituted C6 to C60 aryl group; or a substituted or unsubstituted C2 to C60 heteroaryl group, and
R40 to R47 are the same as or different from each other, and are each independently hydrogen; or deuterium.

4. The heterocyclic compound of claim 1, wherein 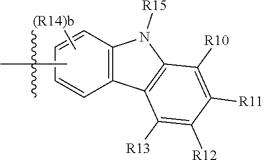
of Chemical Formula A is represented by any one of the following Chemical Formulae 1A to 4A: 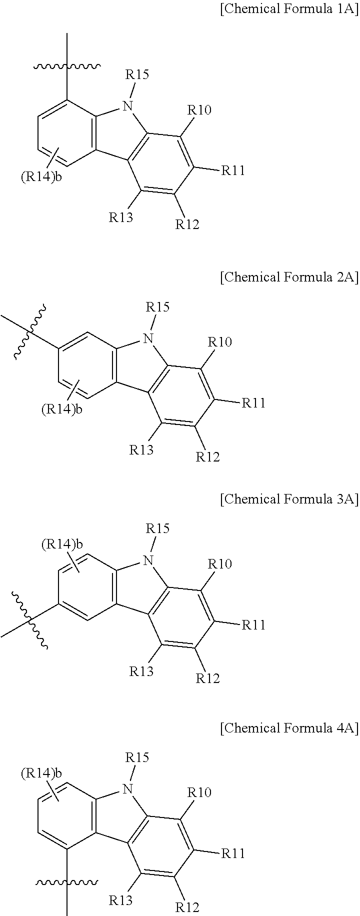
in Chemical Formulae 1A to 4A, 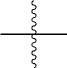
is a position linked to Chemical Formula 1.

5. The heterocyclic compound of claim 1, wherein a deuterium content of Chemical Formula 1 is 0%, or 50% or more and 100% or less.

6. The heterocyclic compound of claim 1, wherein Chemical Formula 1 is represented by any one of the following compounds:

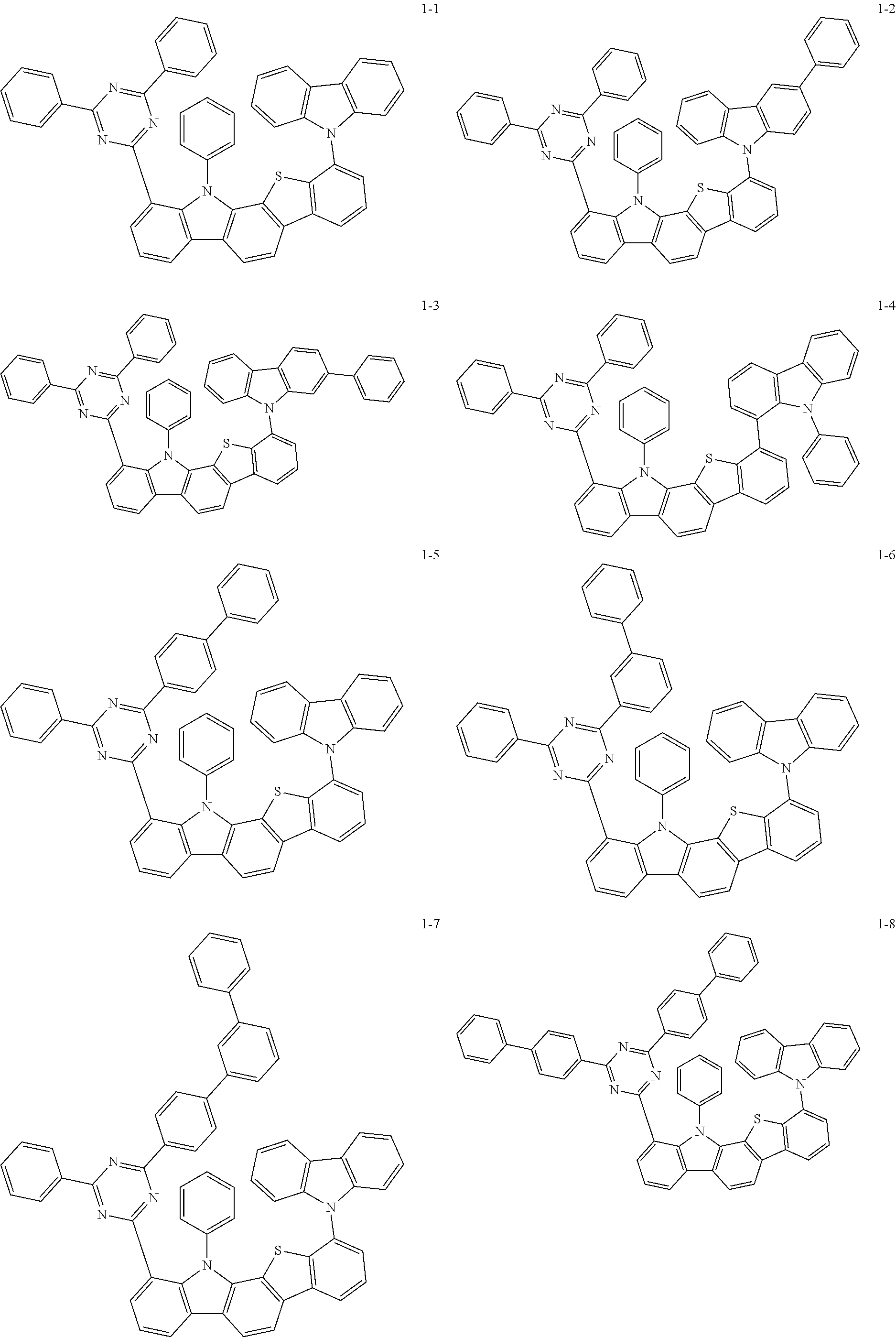

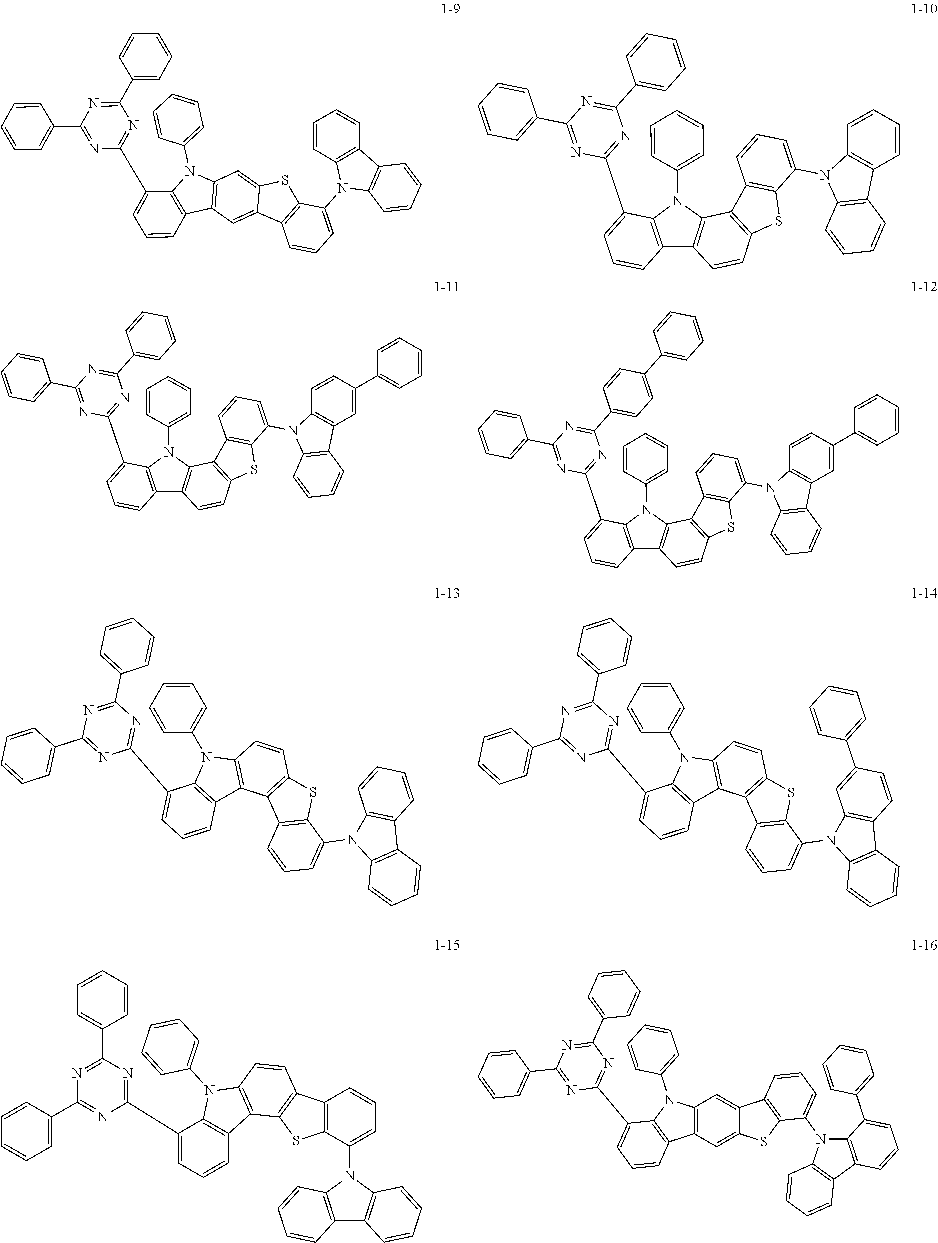

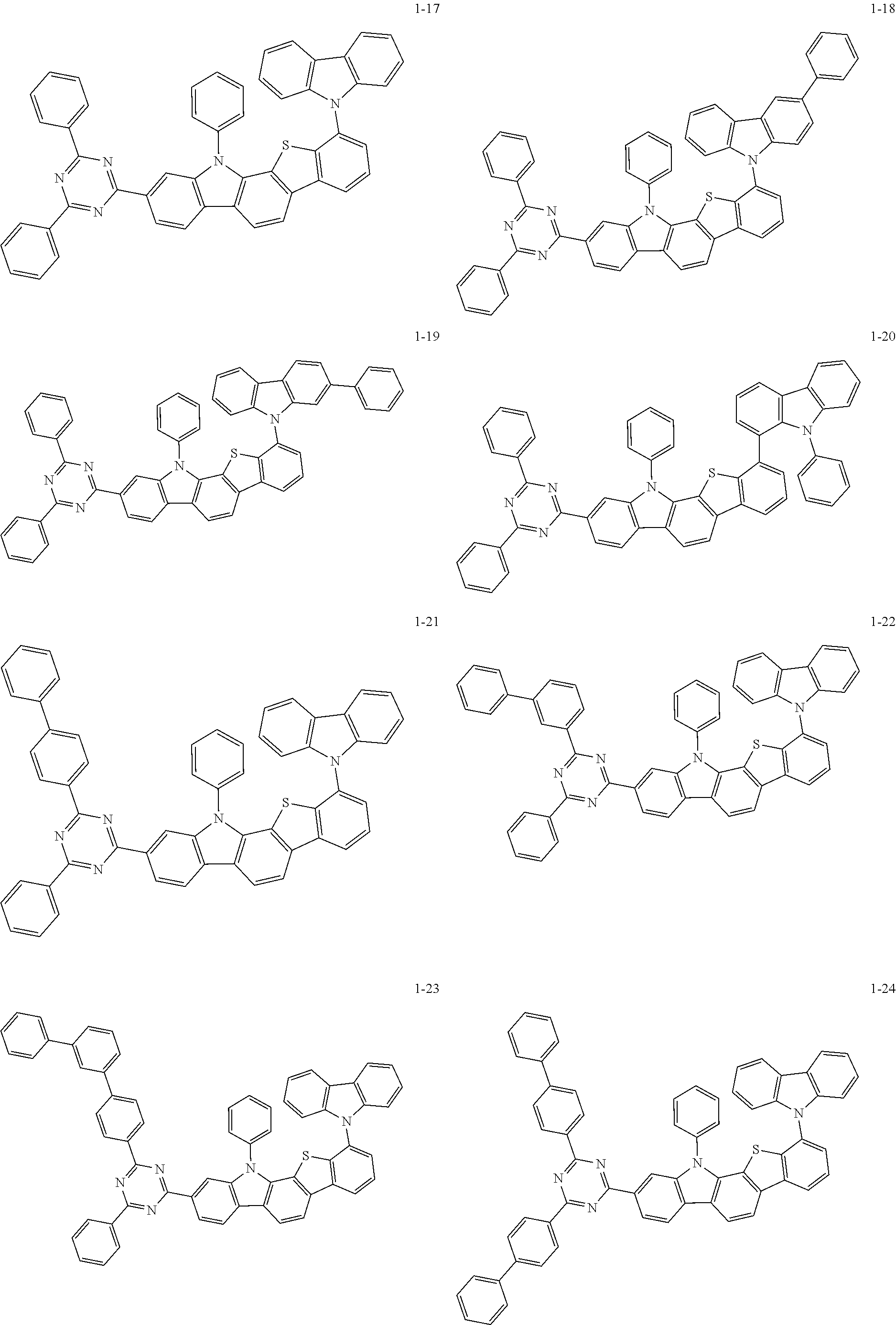

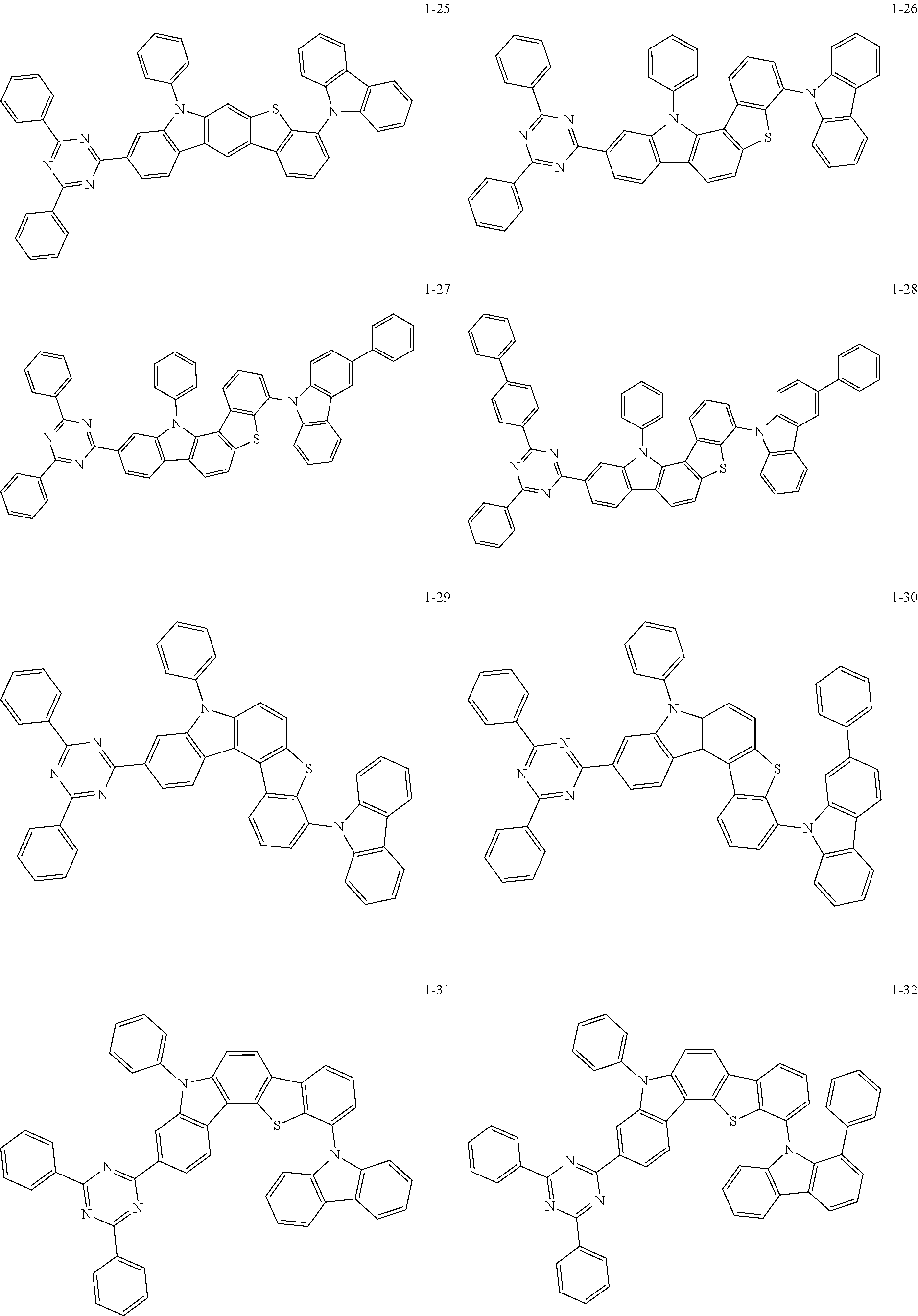

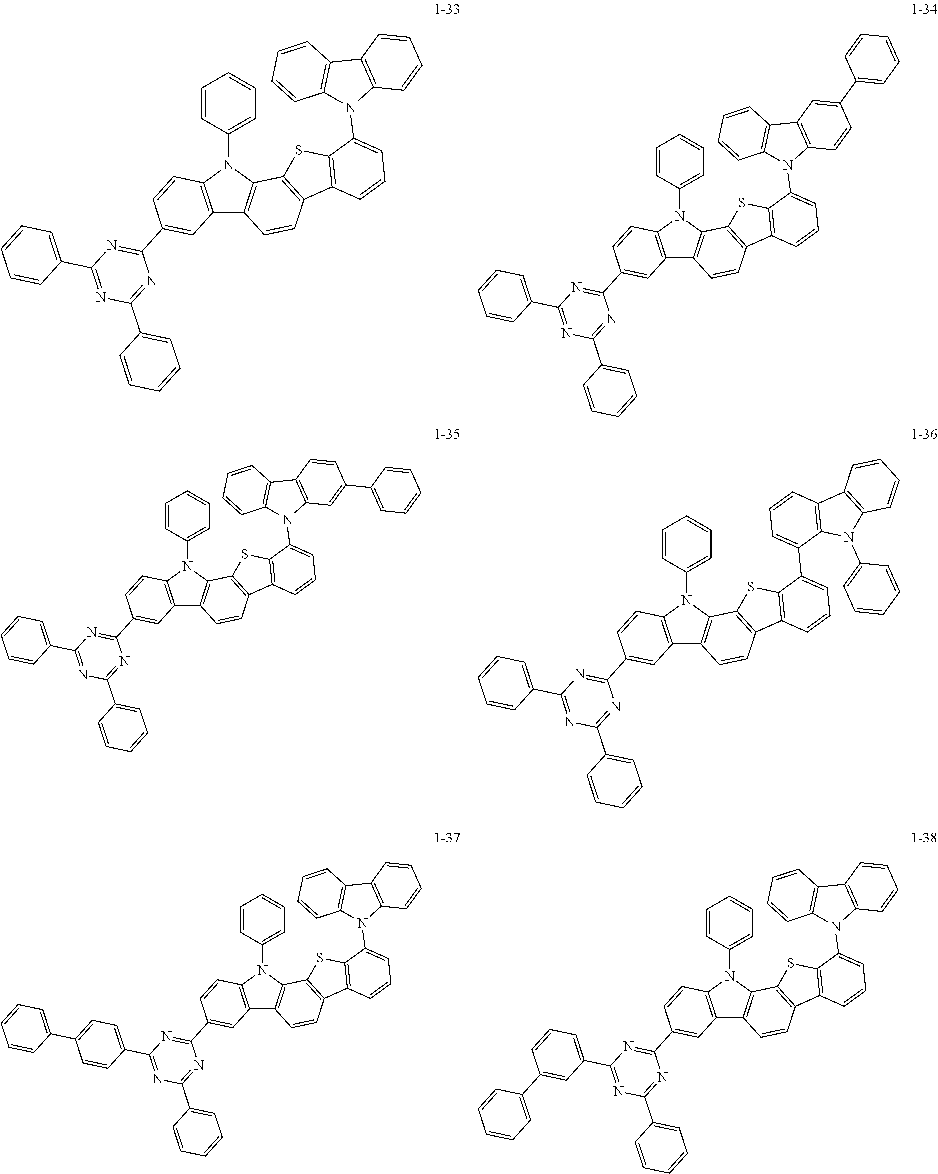

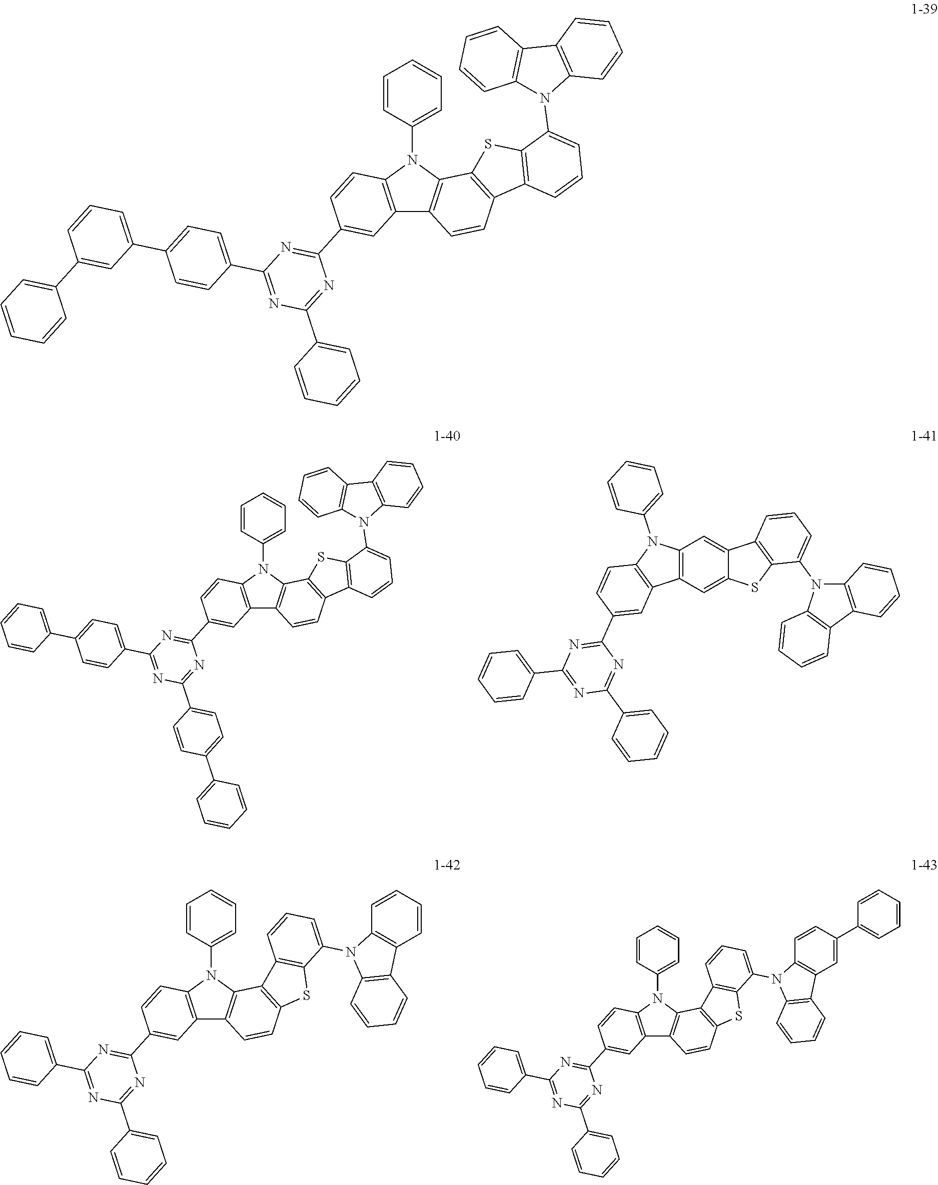

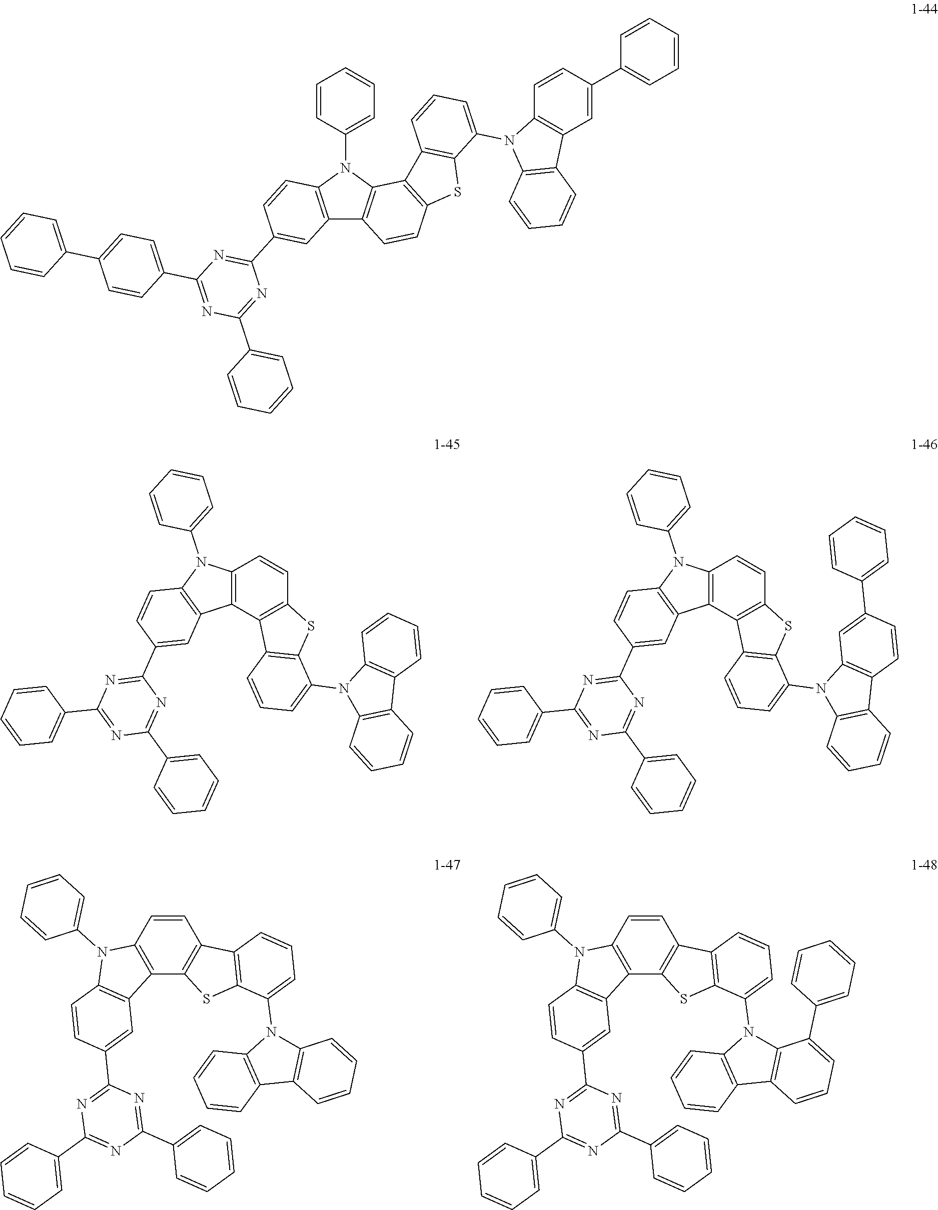

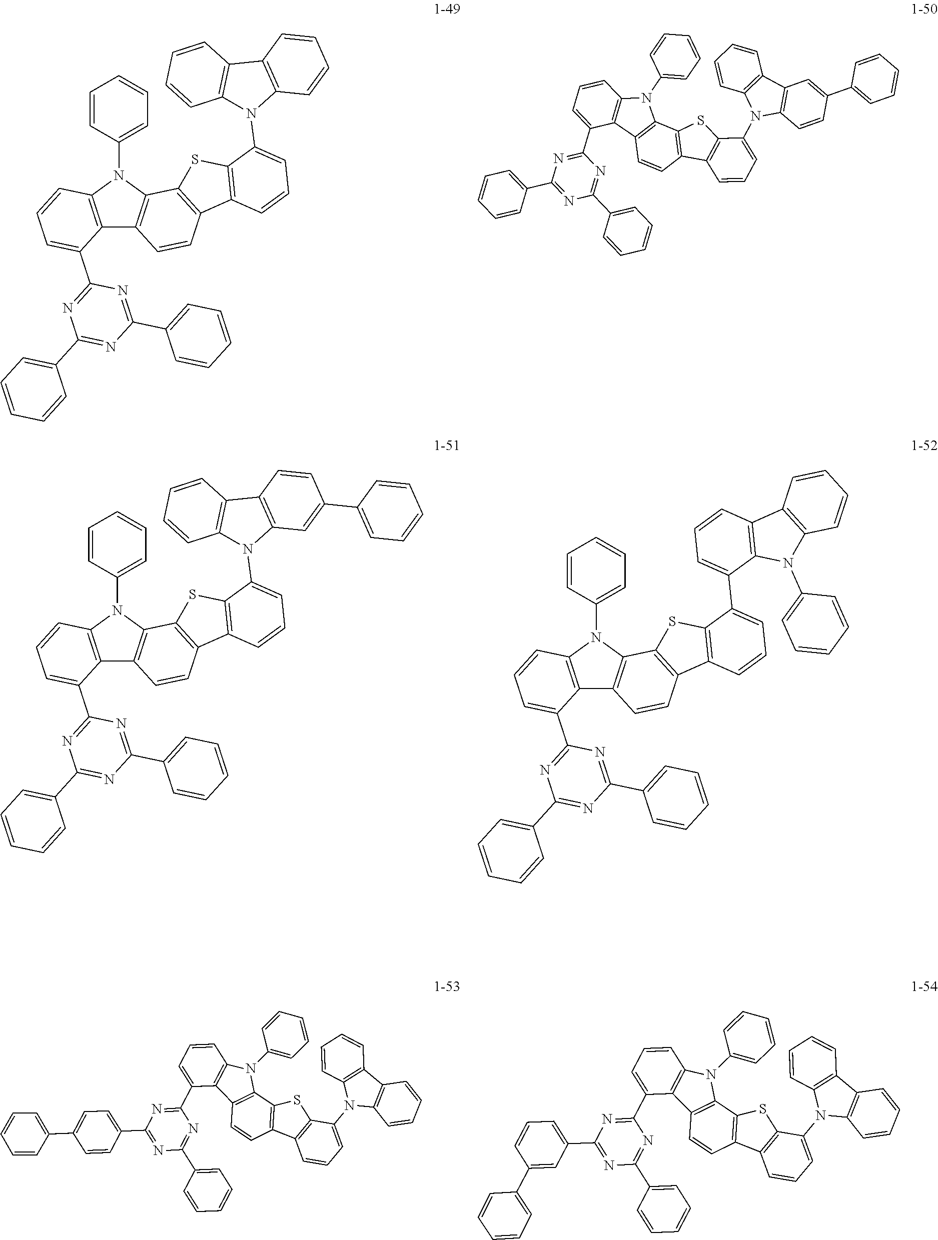

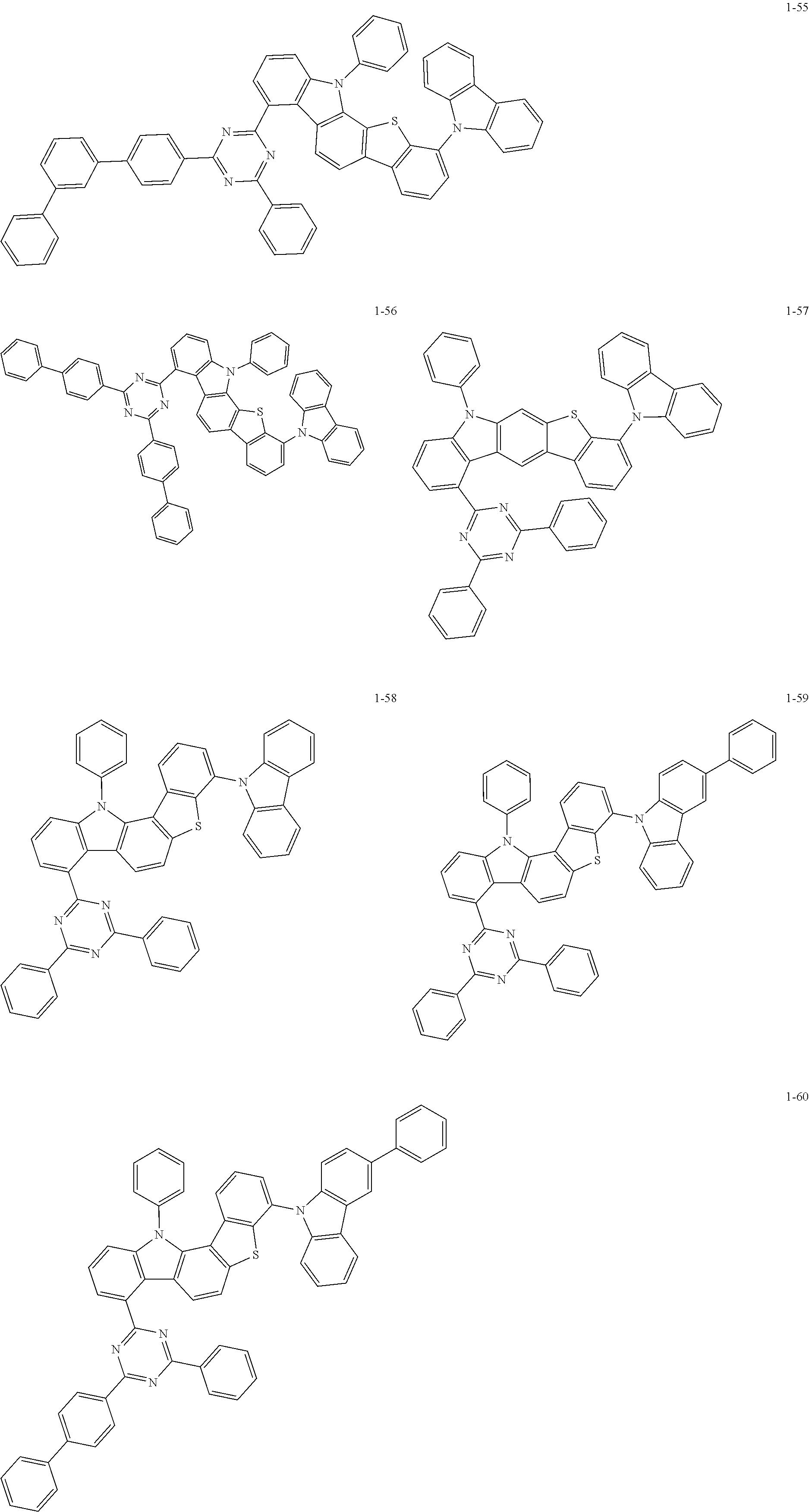

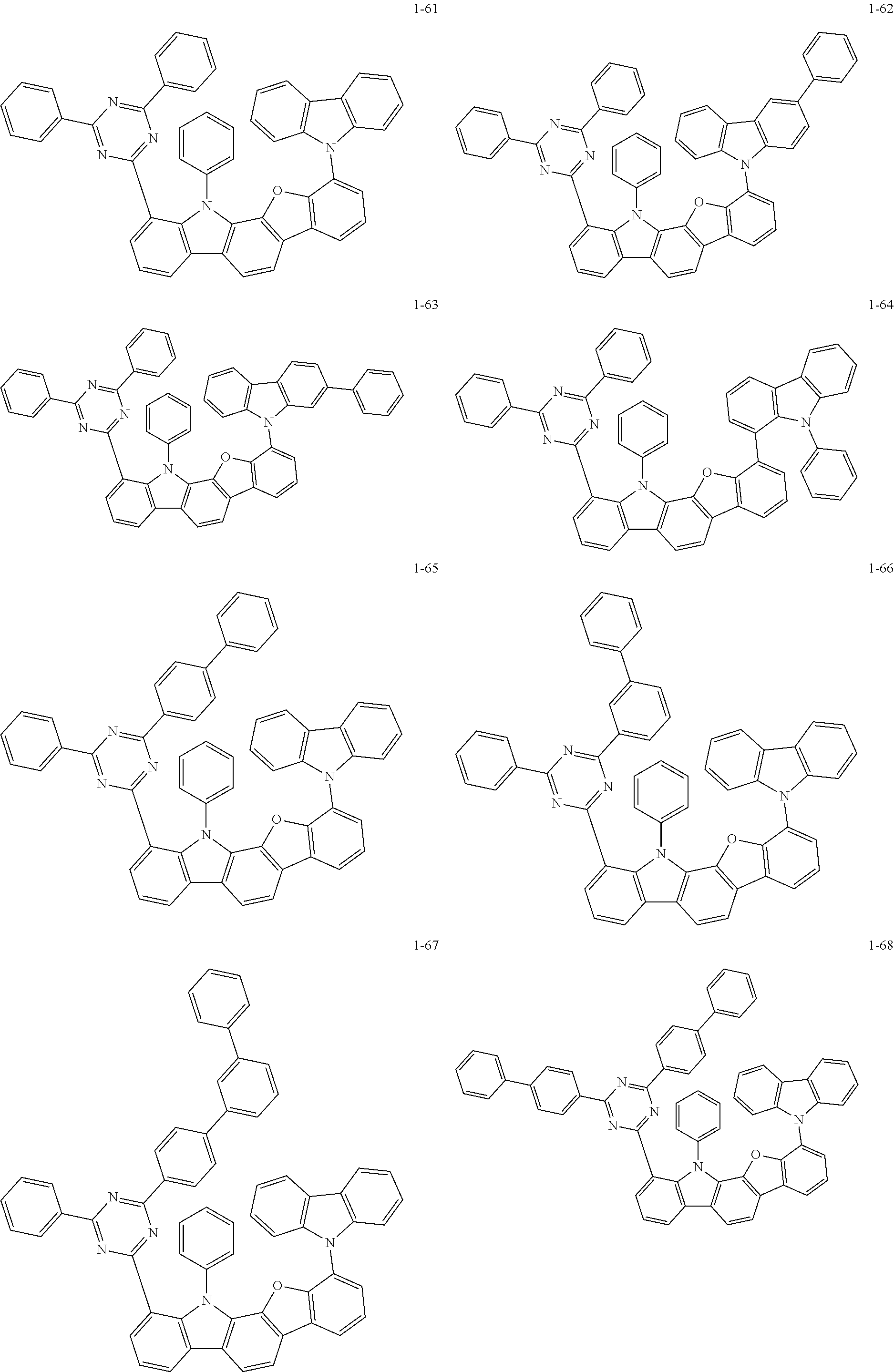

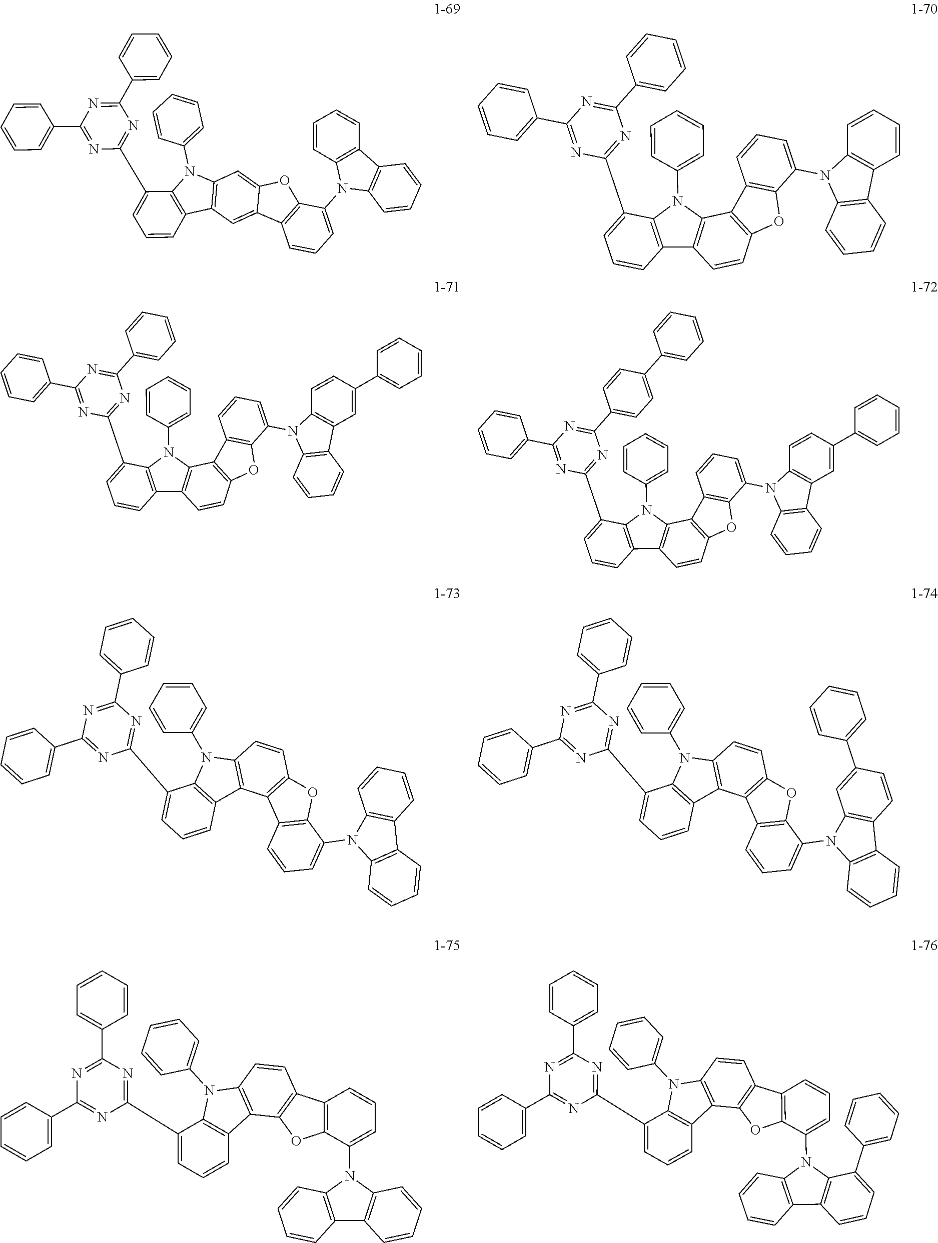

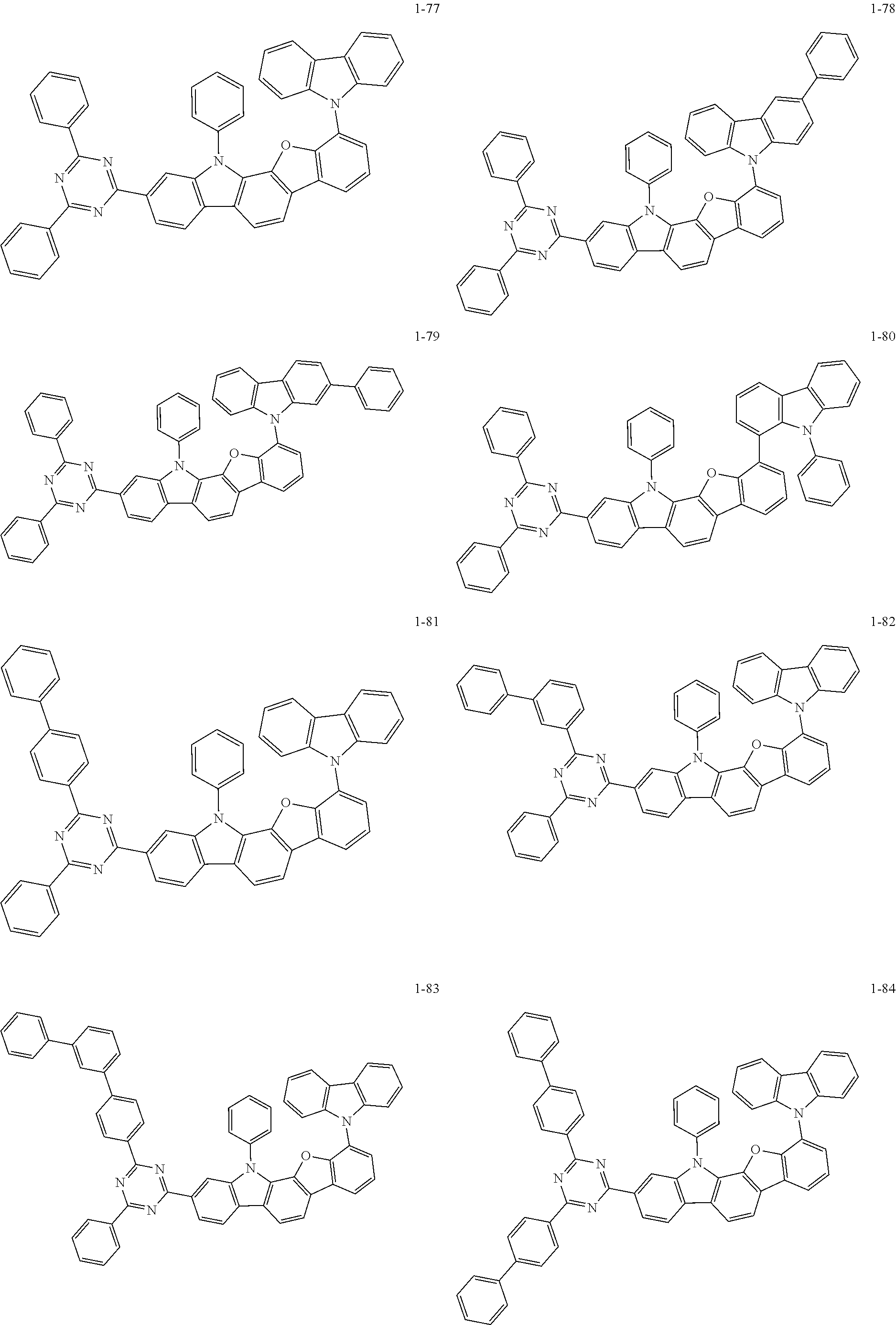

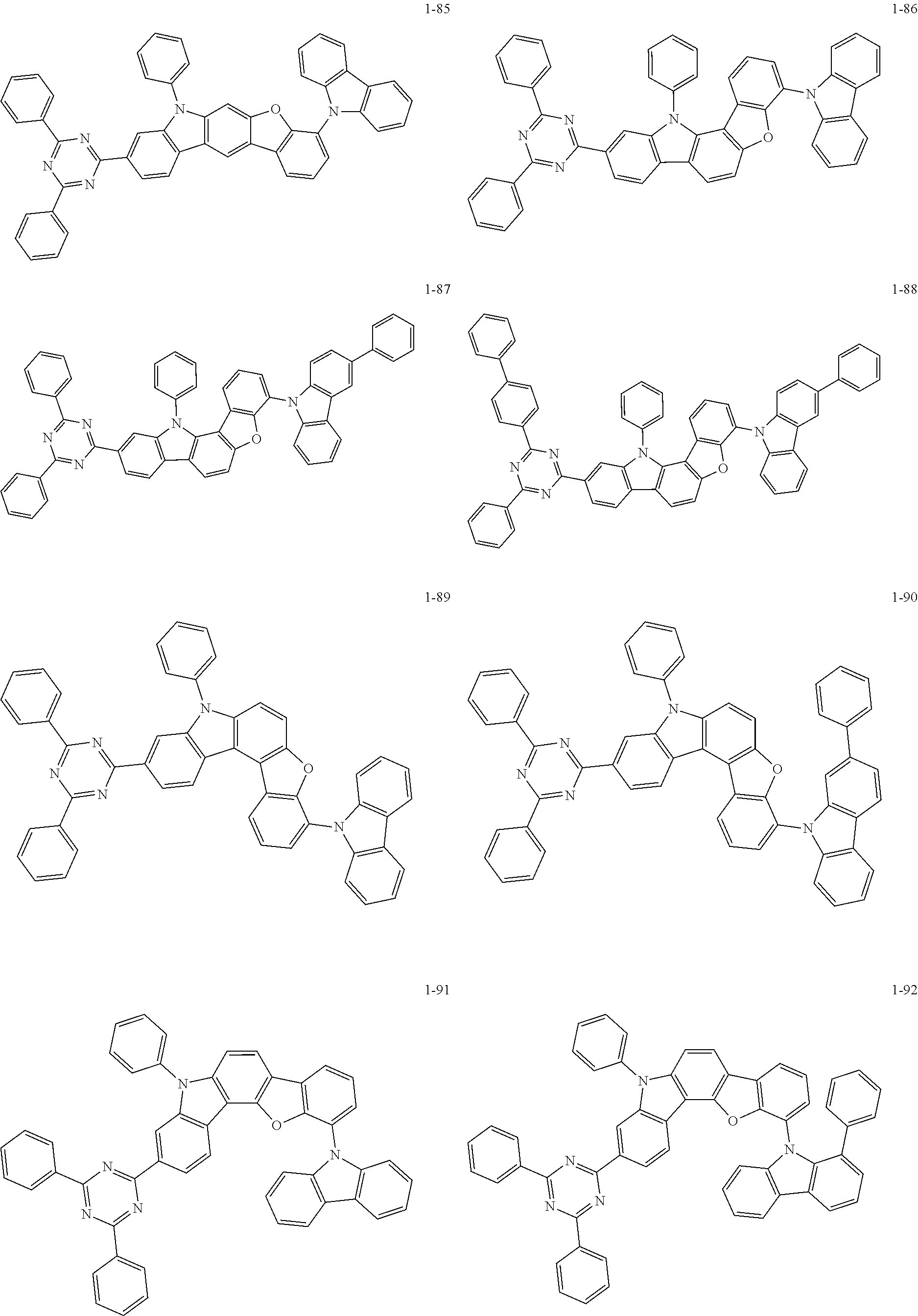

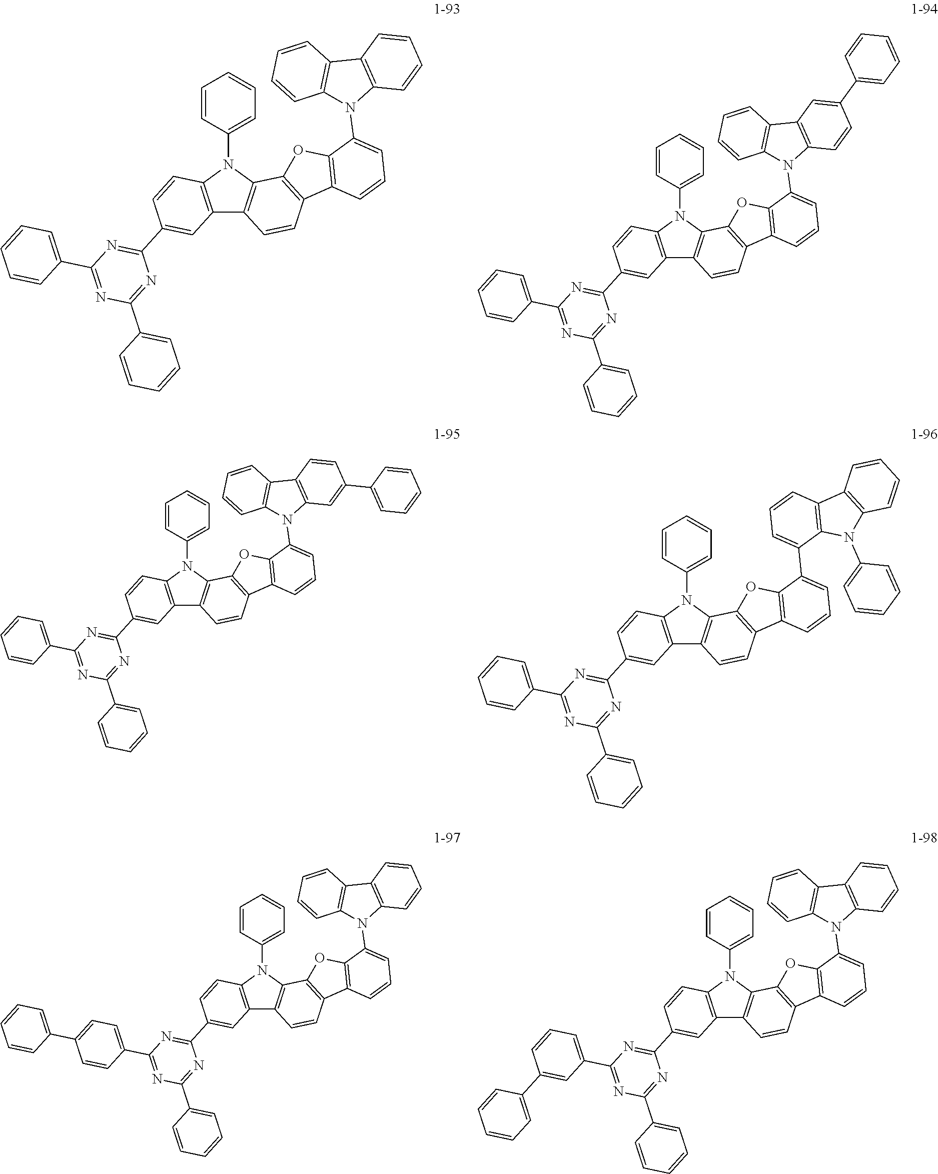

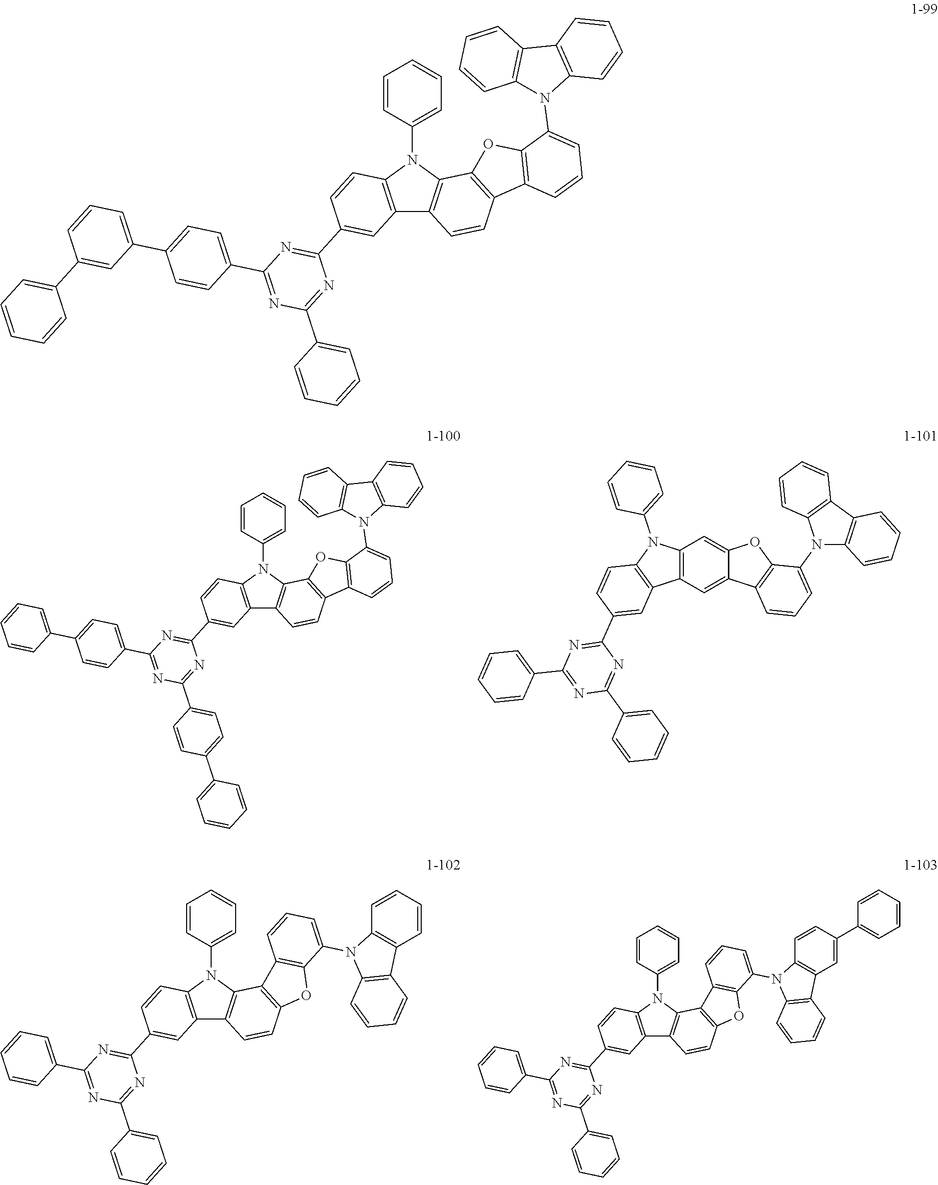

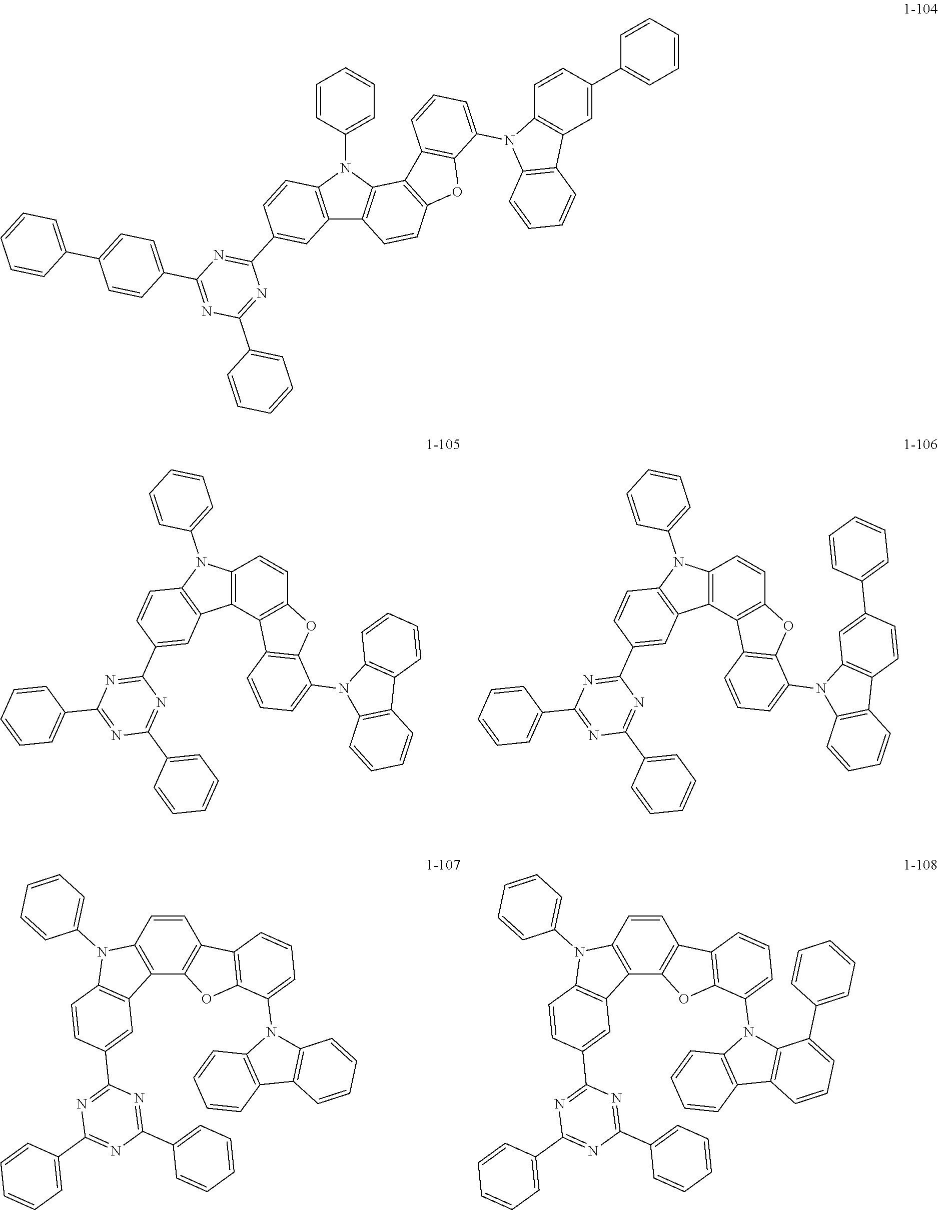

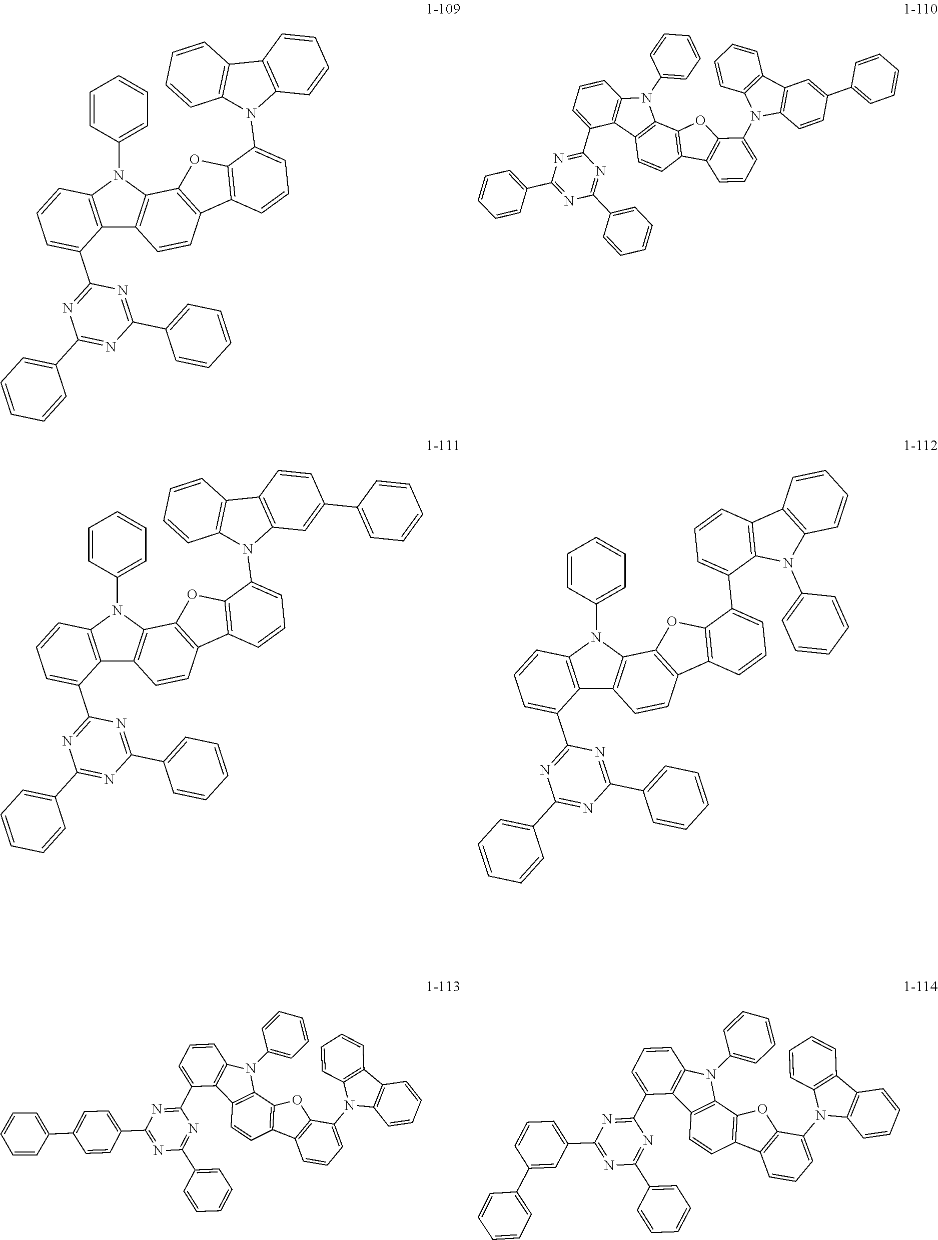

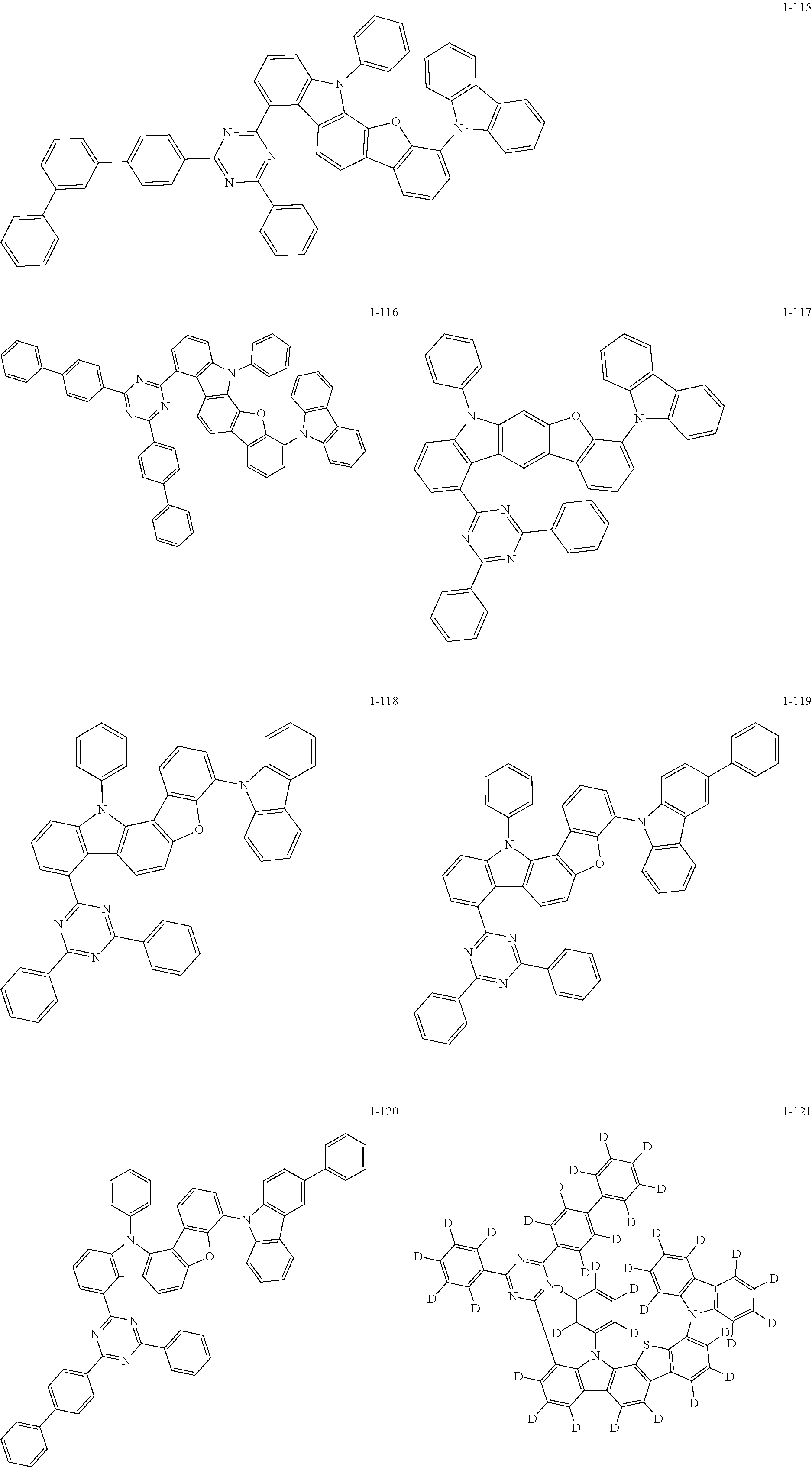

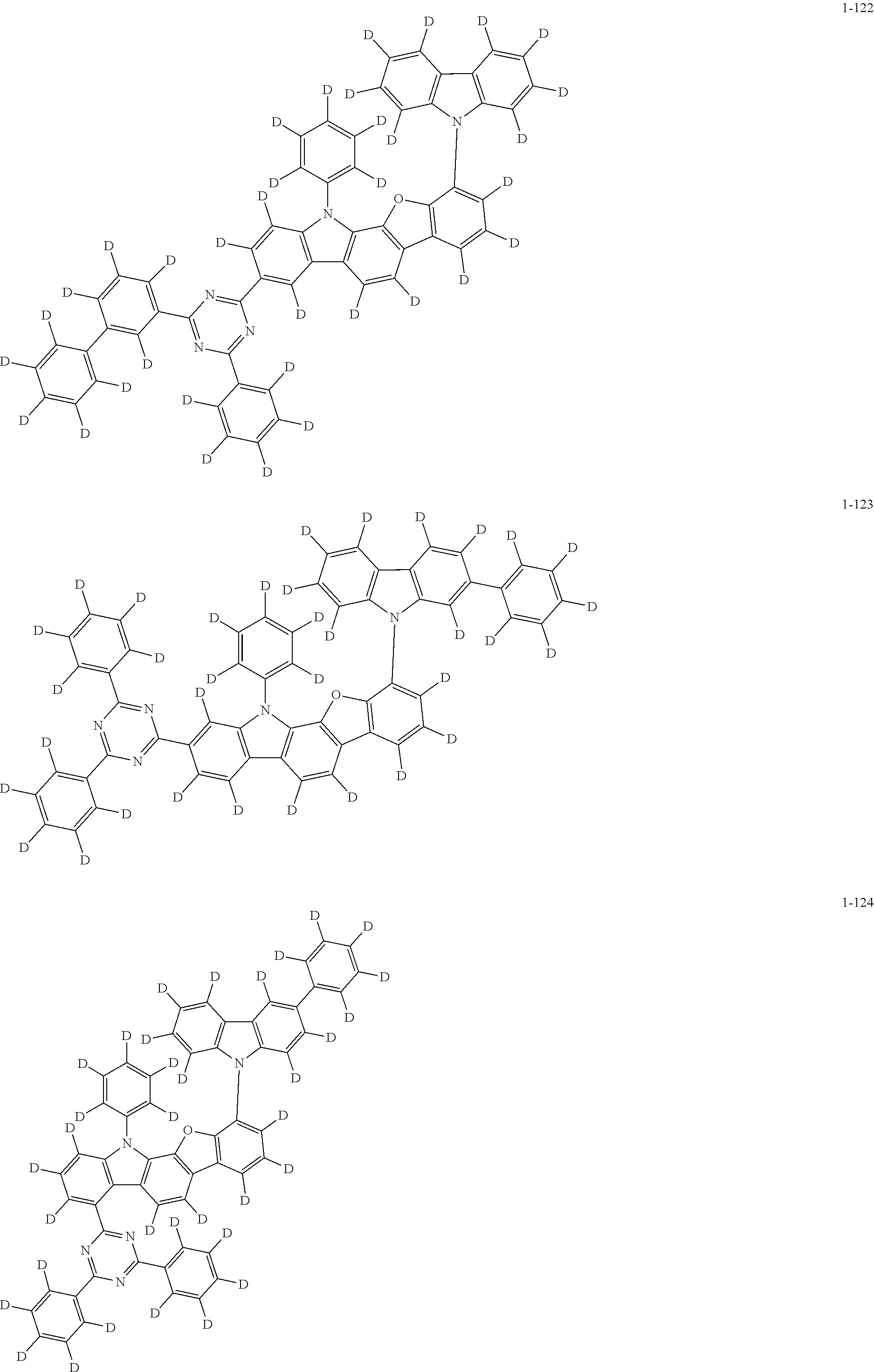

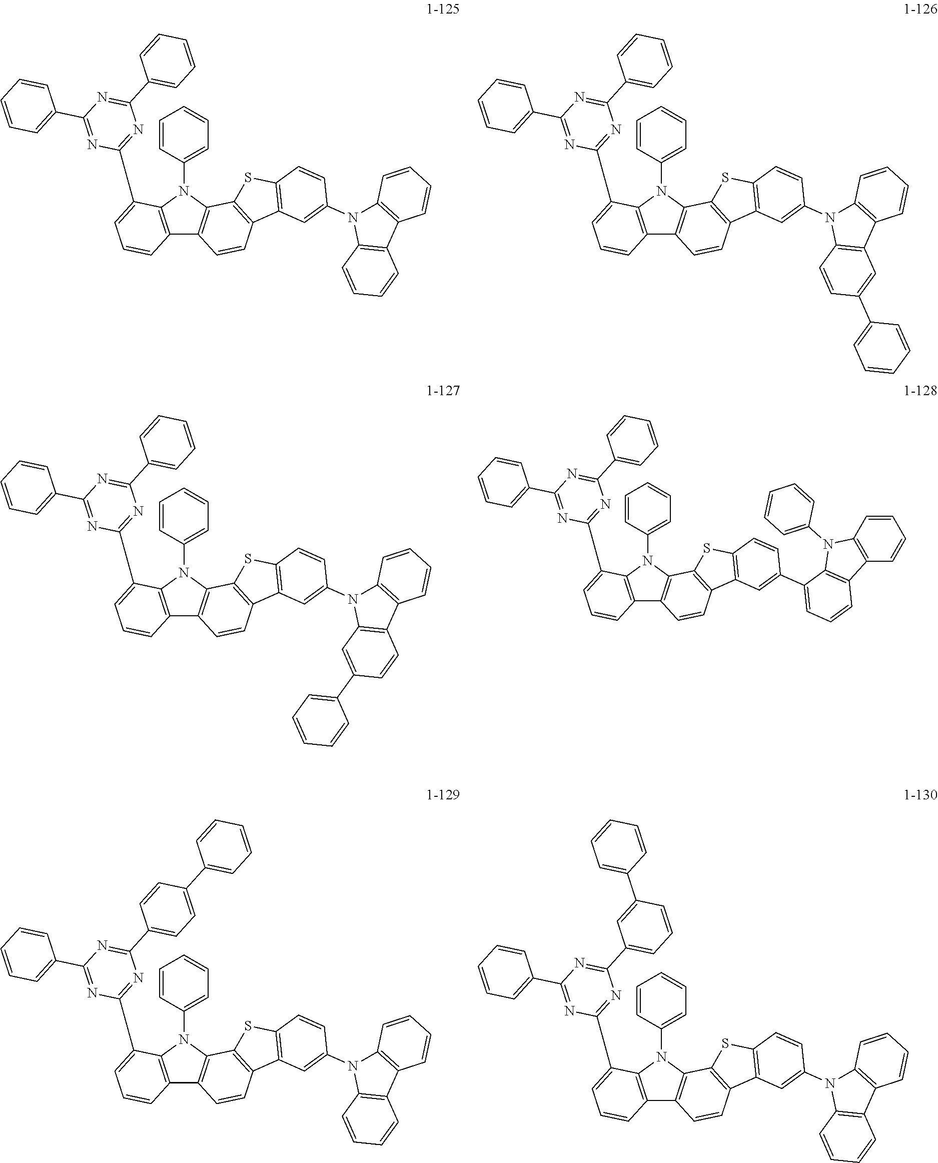

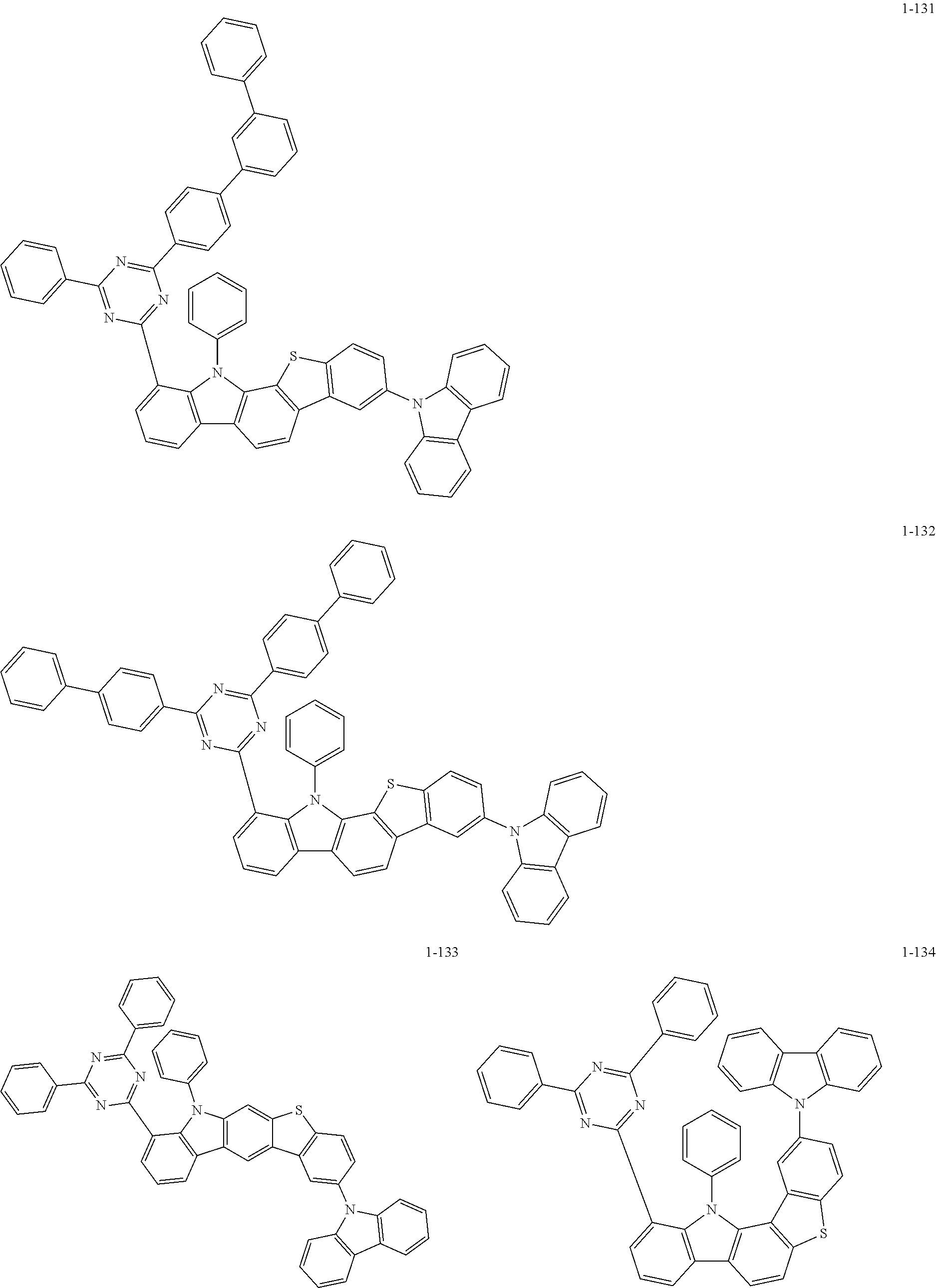 [SMILES: c1ccc(-c2cccc(-c3ccc(-c4nc(-c5ccccc5)nc(-c5cccc6c7ccc8c9cc(-n%10c%11ccccc%11c%11ccccc%11%10)ccc9sc8c7n(-c7ccccc7)c56)n4)cc3)c2)cc1 ; c1ccc(-c2ccc(-c3nc(-c4ccc(-c5ccccc5)cc4)nc(-c4cccc5c6ccc7c8cc(-n9c%10ccccc%10c%10ccccc%109)ccc8sc7c6n(-c6ccccc6)c45)n3)cc2)cc1 ; c1ccc(-c2nc(-c3ccccc3)nc(-c3cccc4c5cc6c(cc5n(-c5ccccc5)c34)sc3ccc(-n4c5ccccc5c5ccccc54)cc36)n2)cc1 ; c1ccc(-c2nc(-c3ccccc3)nc(-c3cccc4c5ccc6sc7ccc(-n8c9ccccc9c9ccccc98)cc7c6c5n(-c5ccccc5)c34)n2)cc1]

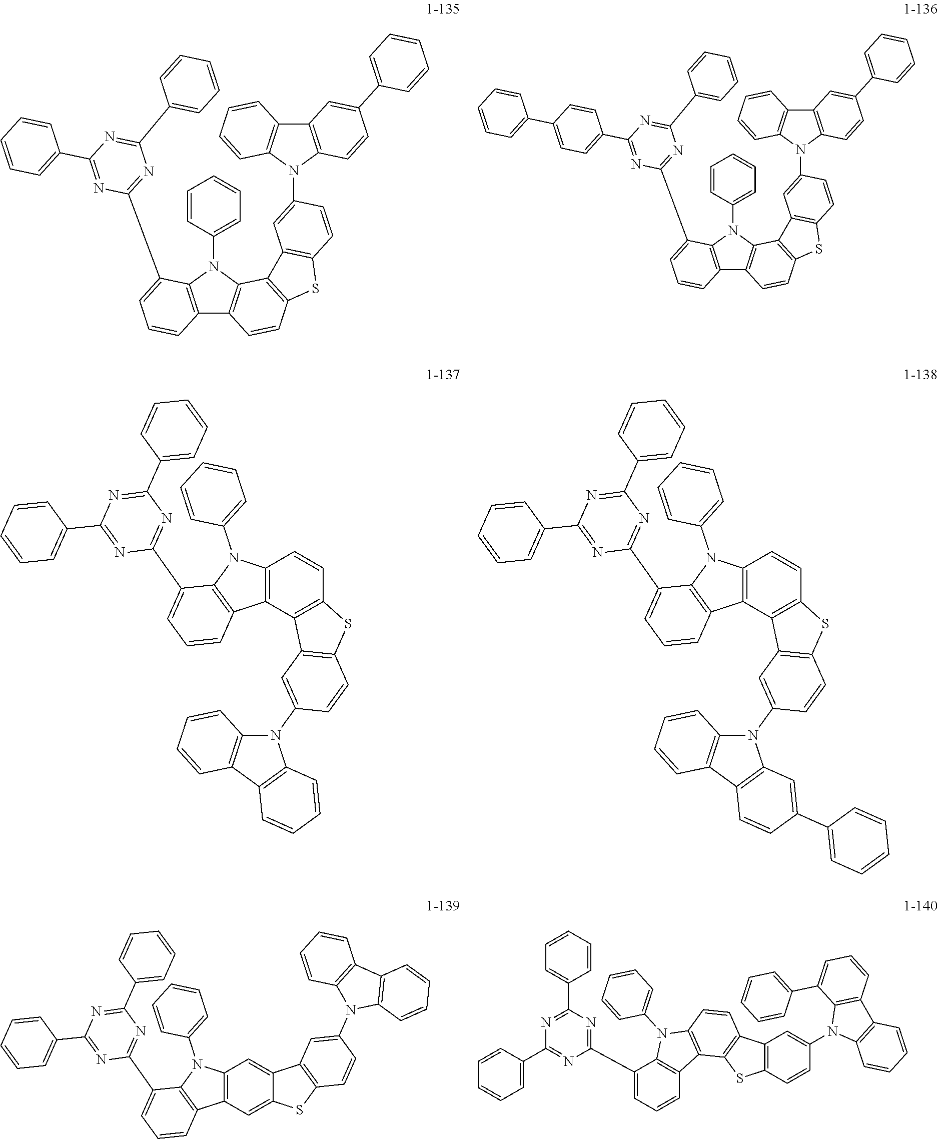

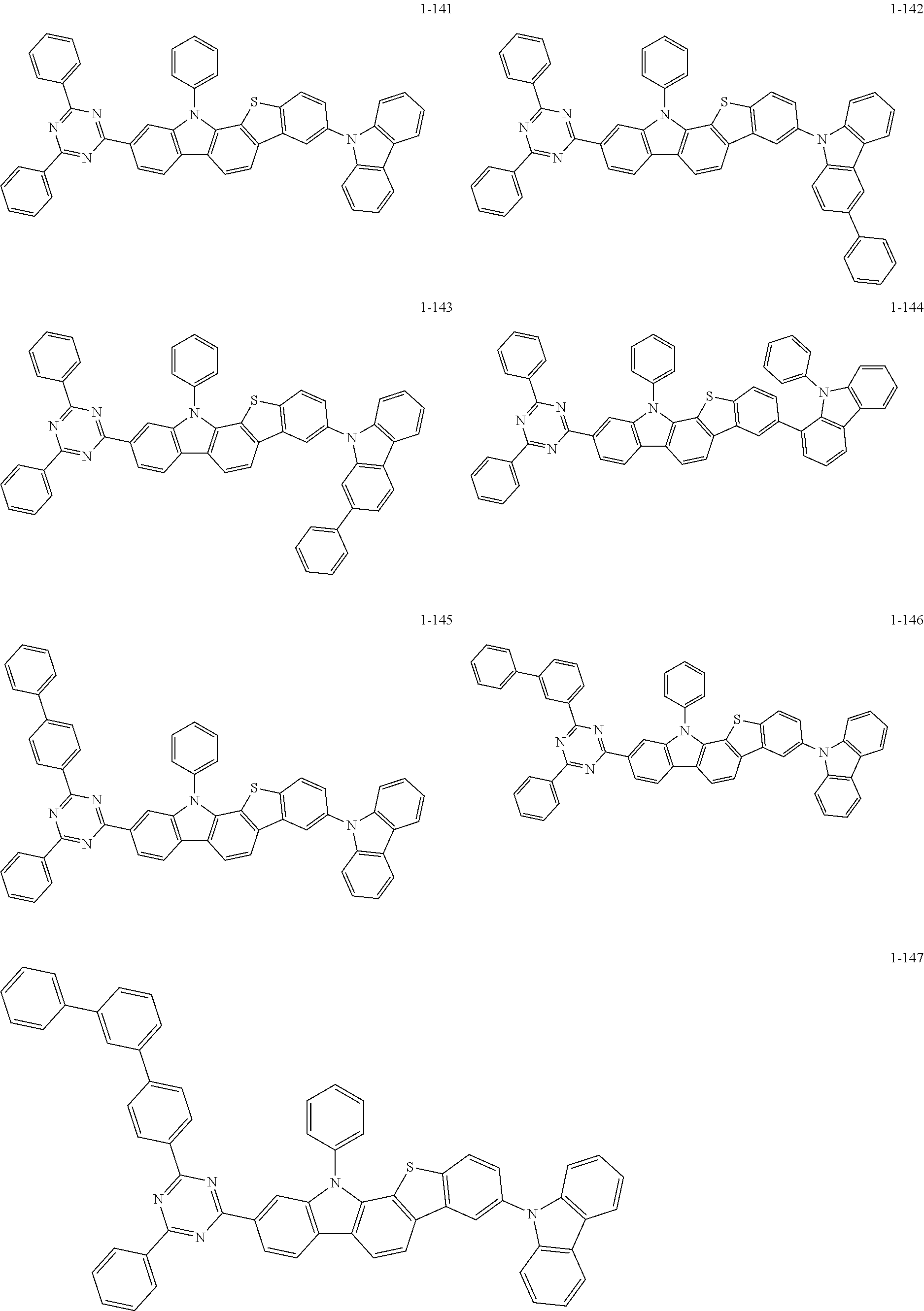

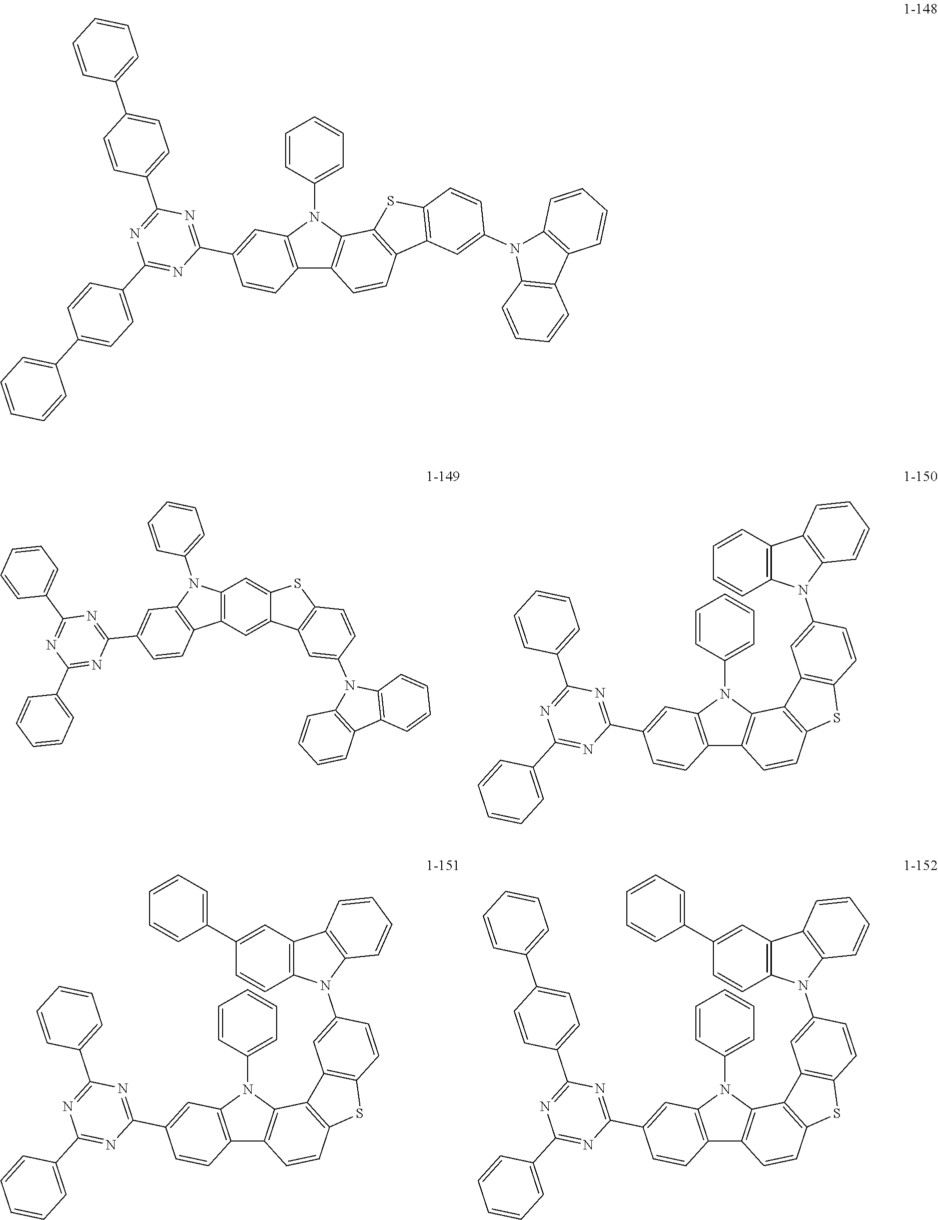

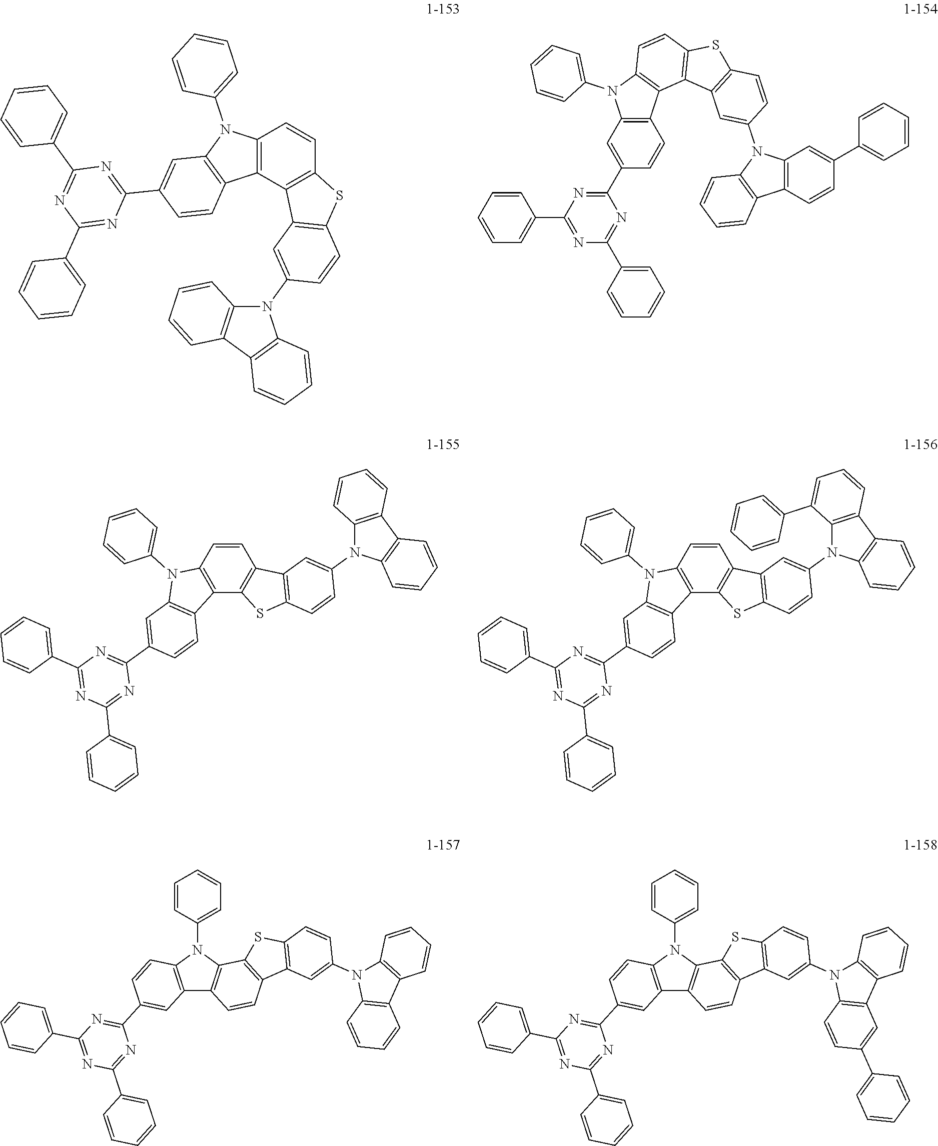

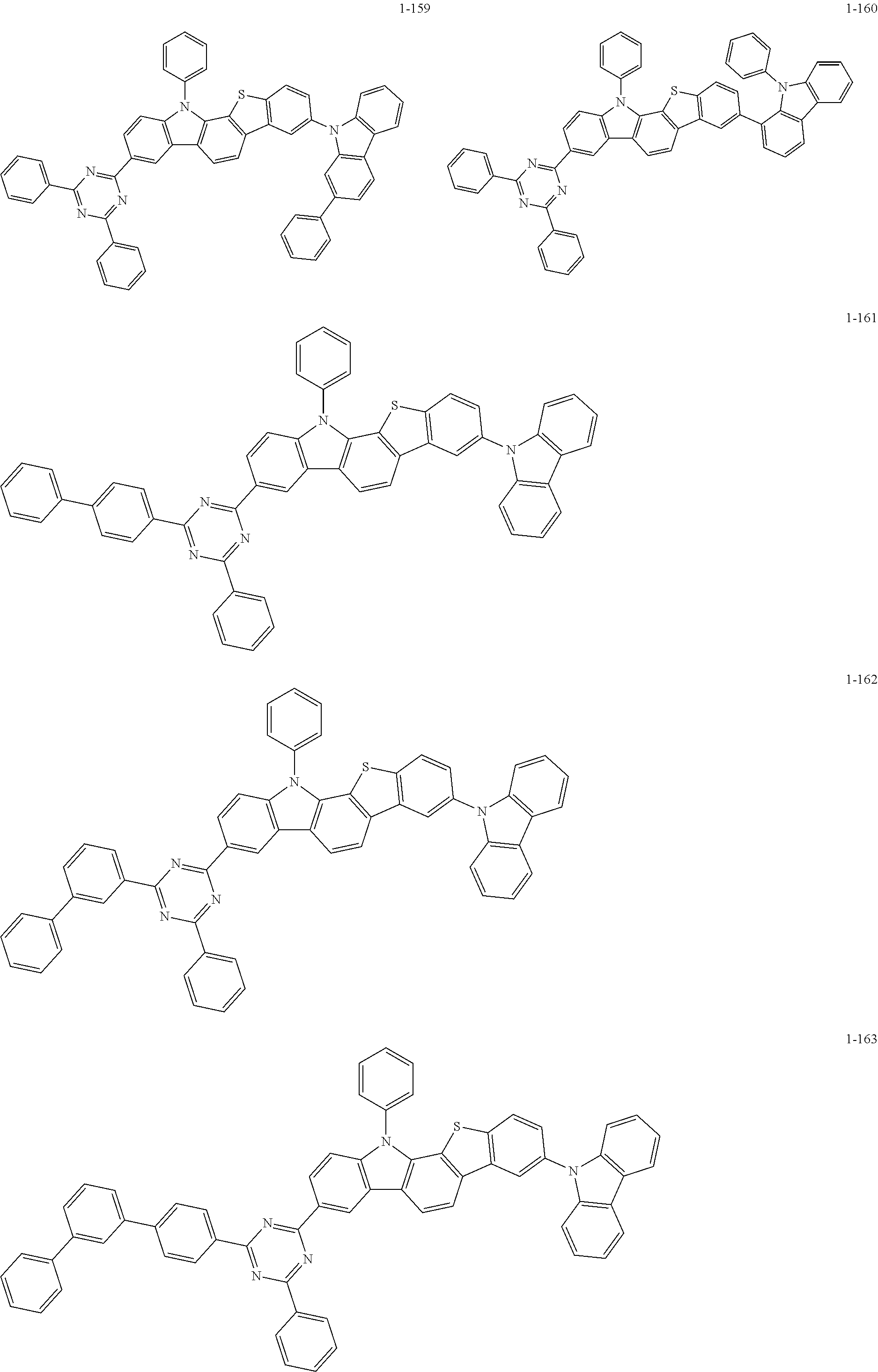

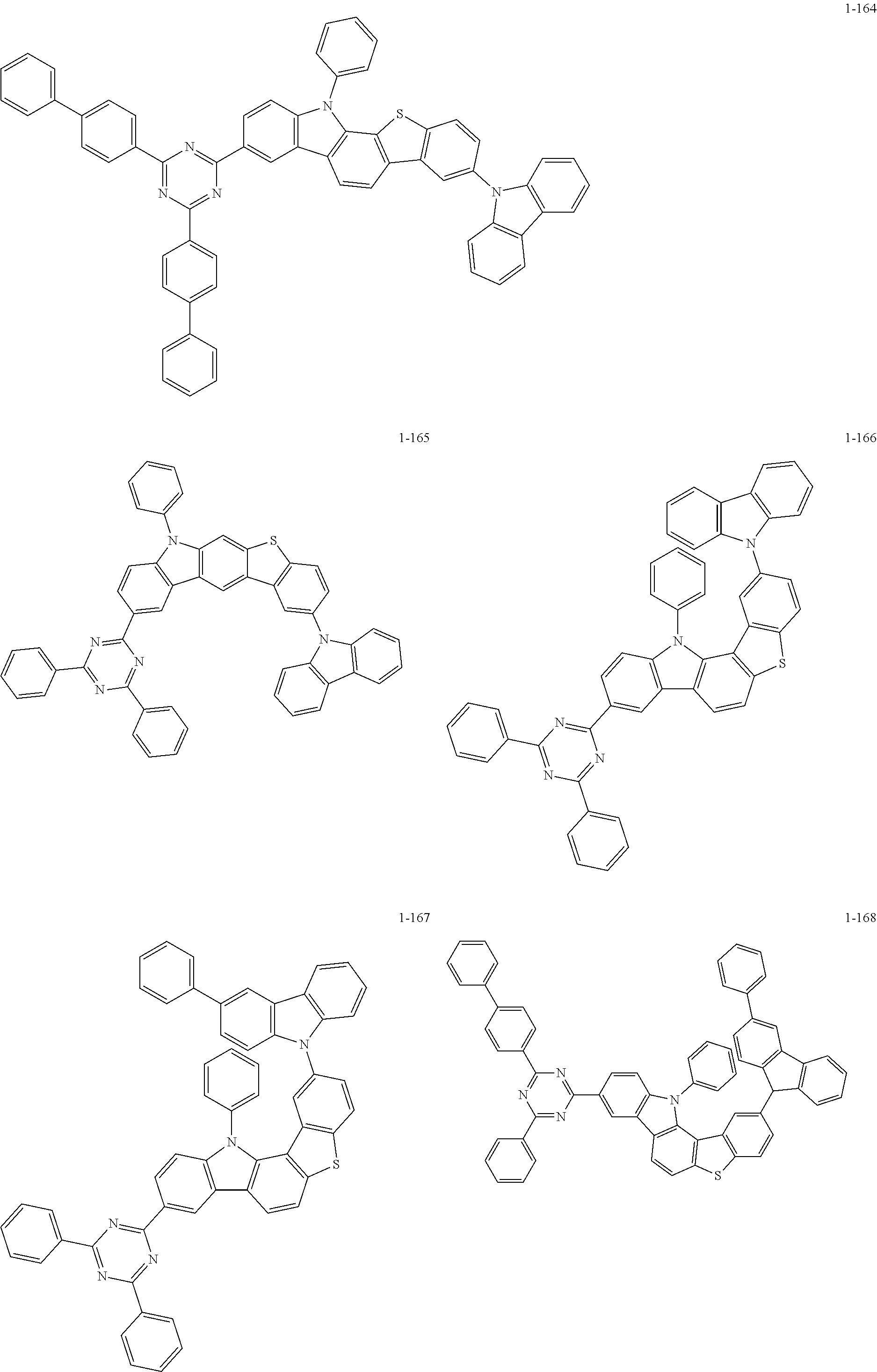

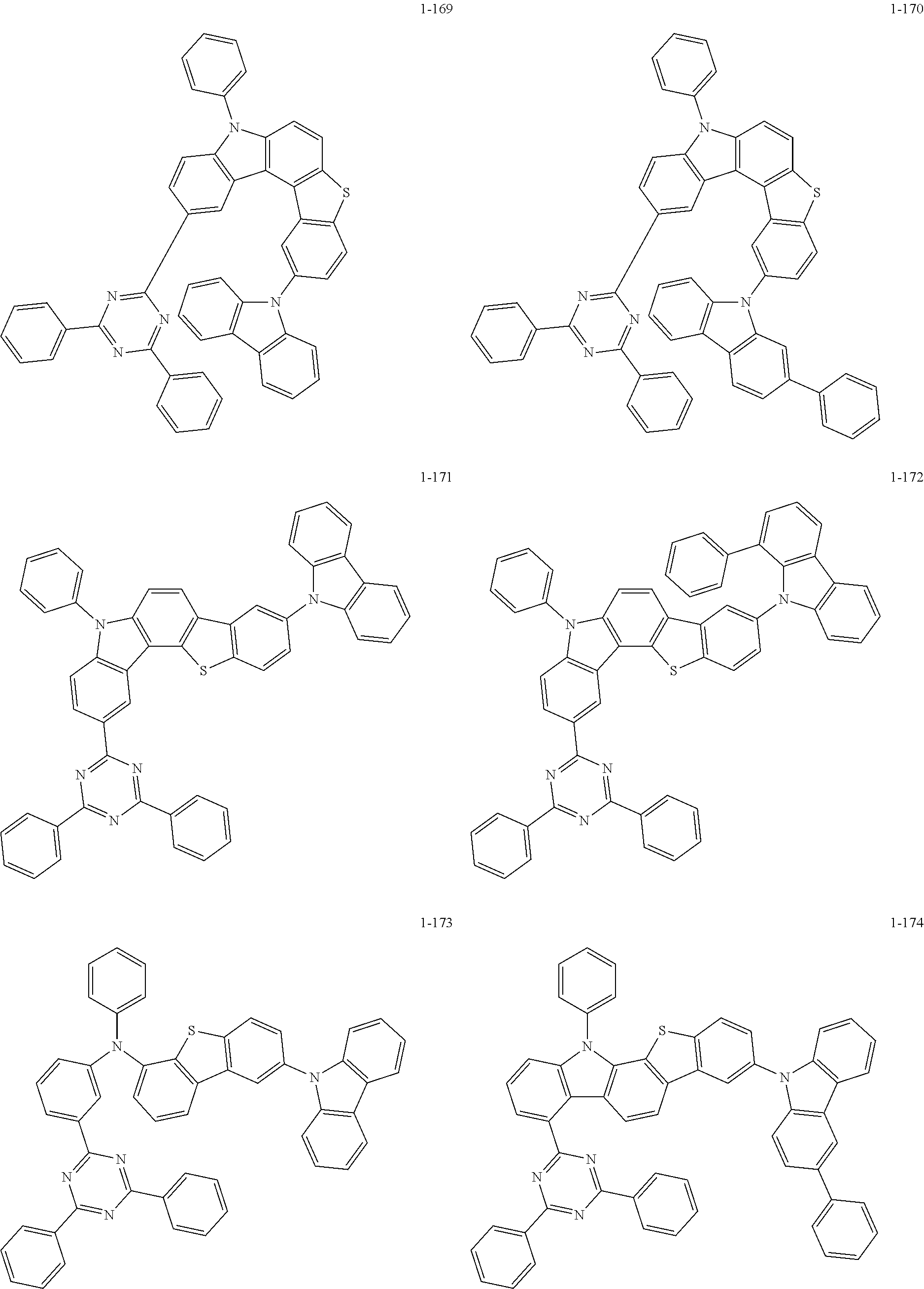

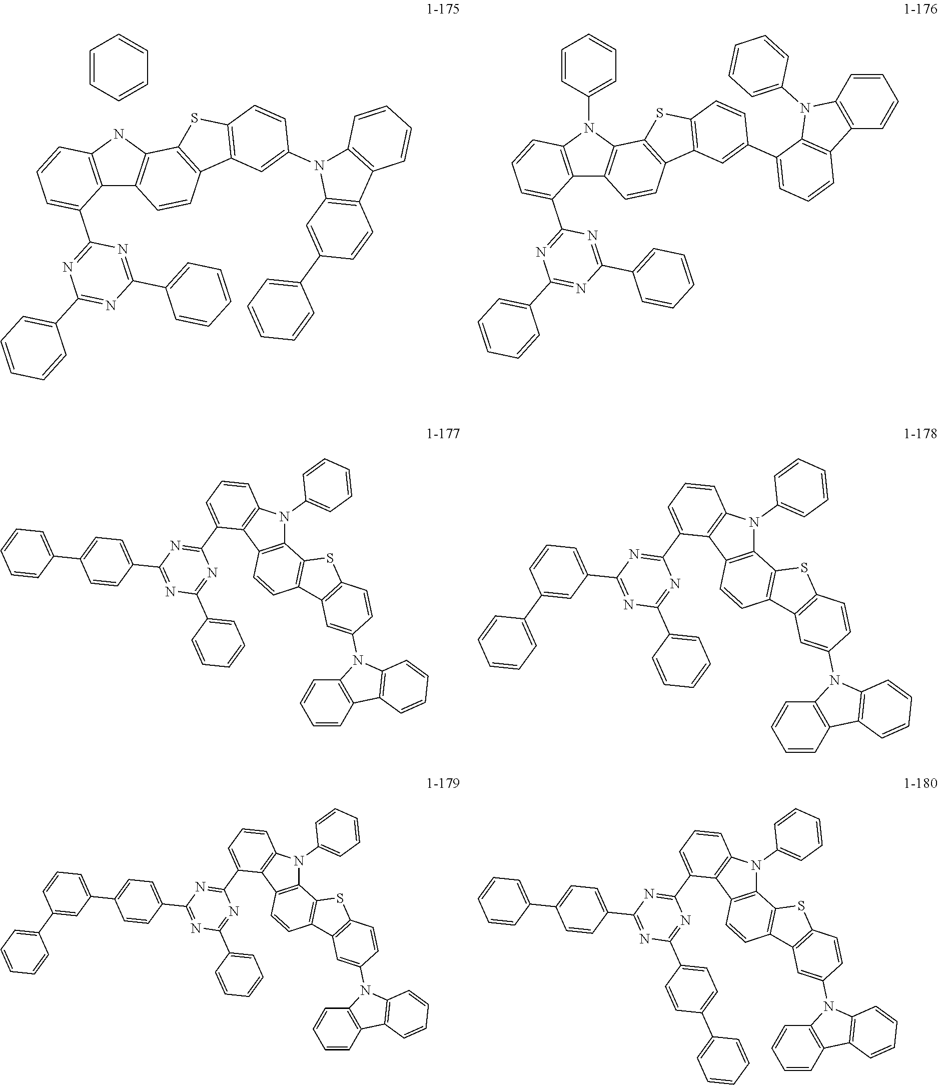

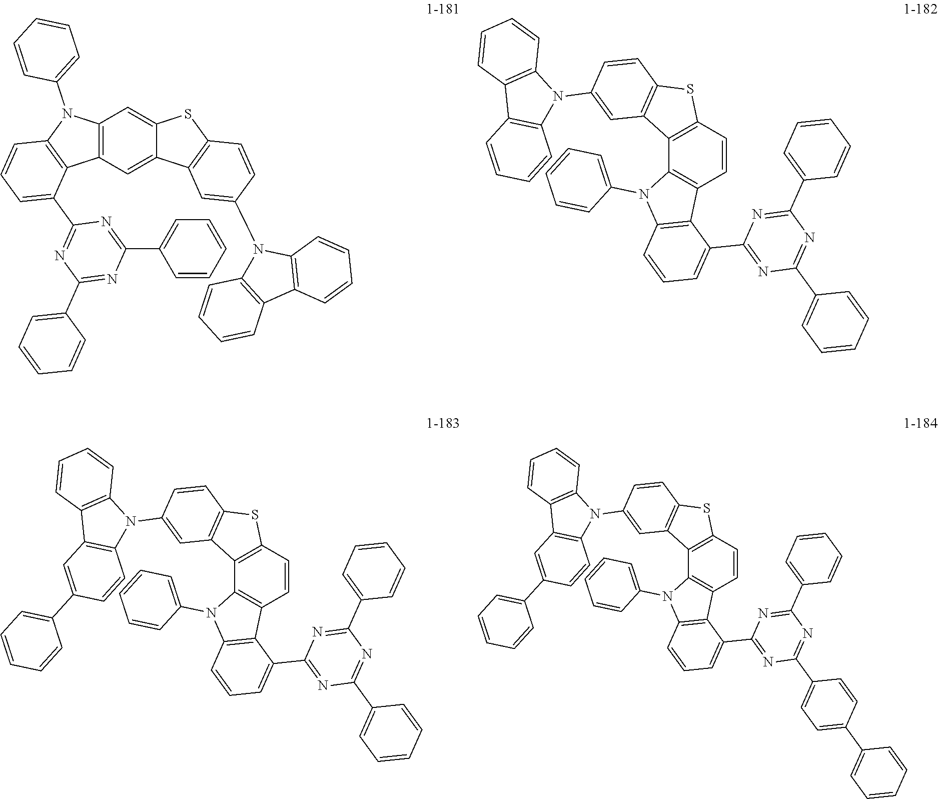 [SMILES: c1ccc(-c2nc(-c3ccccc3)nc(-c3cccc4c3c3cc5c(cc3n4-c3ccccc3)sc3ccc(-n4c6ccccc6c6ccccc64)cc35)n2)cc1 ; c1ccc(-c2nc(-c3ccccc3)nc(-c3cccc4c3c3ccc5sc6ccc(-n7c8ccccc8c8ccccc87)cc6c5c3n4-c3ccccc3)n2)cc1 ; c1ccc(-c2ccc3c(c2)c2ccccc2n3-c2ccc3sc4ccc5c6c(-c7nc(-c8ccccc8)nc(-c8ccccc8)n7)cccc6n(-c6ccccc6)c5c4c3c2)cc1 ; c1ccc(-c2ccc(-c3nc(-c4ccccc4)nc(-c4cccc5c4c4ccc6sc7ccc(-n8c9ccccc9c9cc(-c%10ccccc%10)ccc98)cc7c6c4n5-c4ccccc4)n3)cc2)cc1]

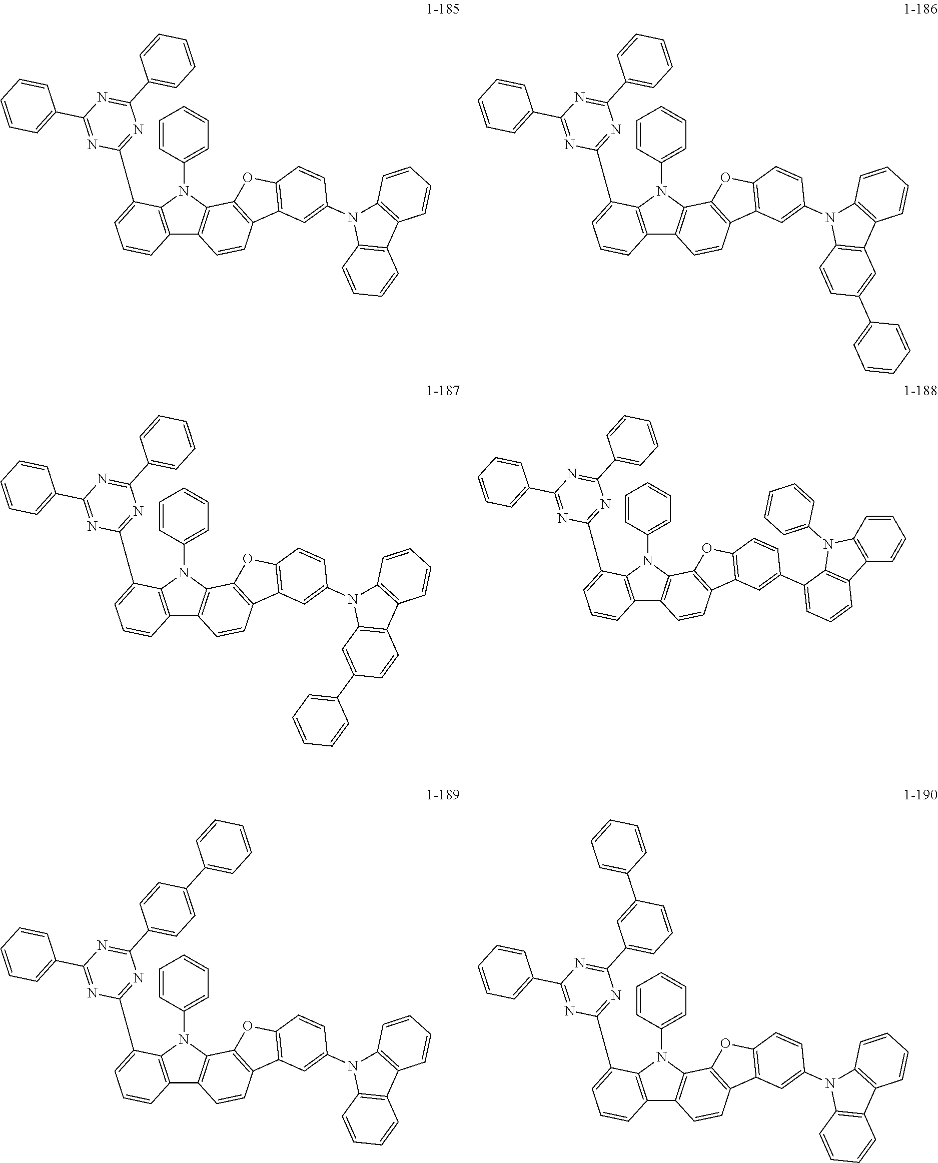

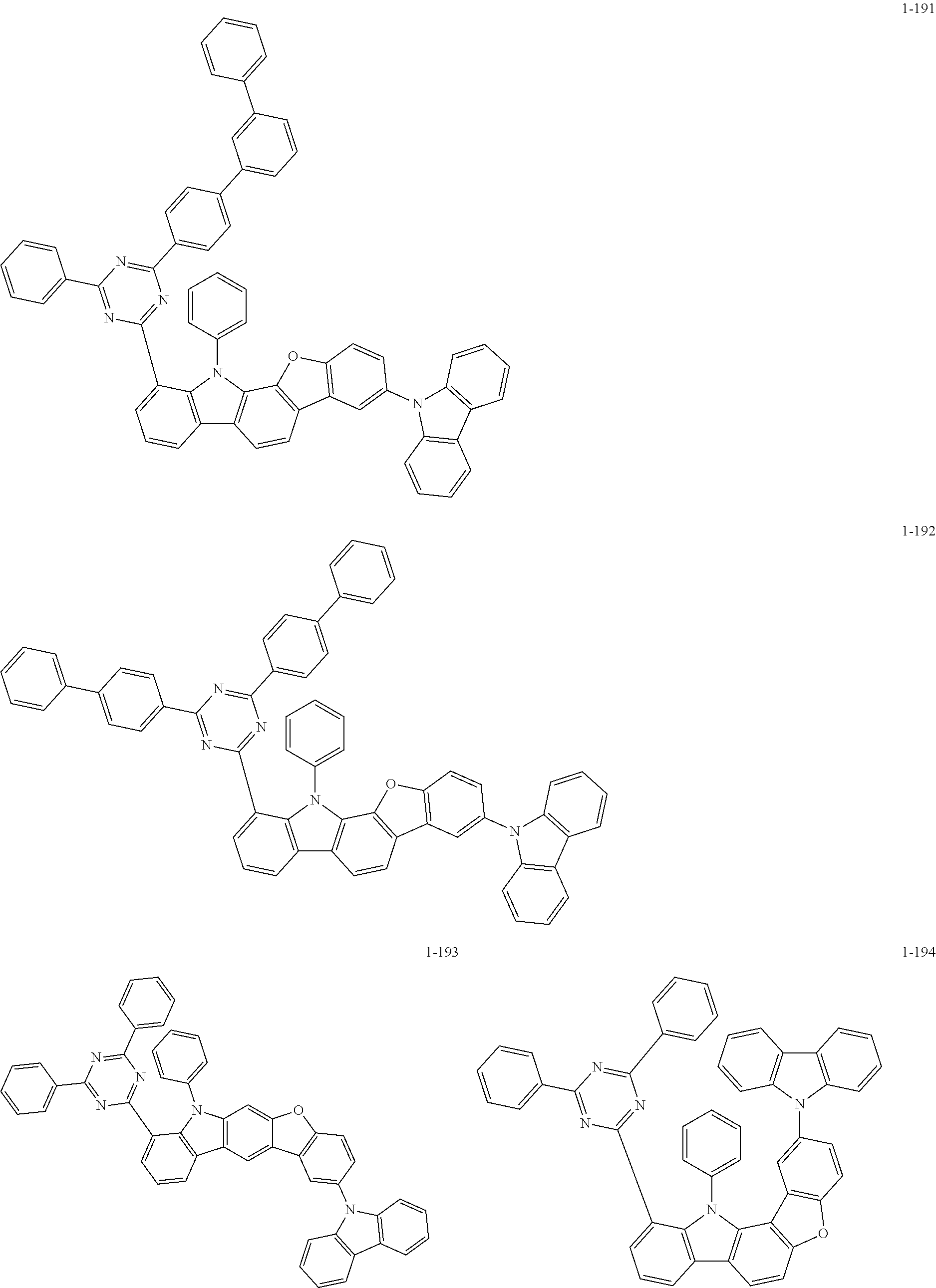 [SMILES: c1ccc(-c2cccc(-c3ccc(-c4nc(-c5ccccc5)nc(-c5cccc6c7ccc8c9cc(-n%10c%11ccccc%11c%11ccccc%11%10)ccc9oc8c7n(-c7ccccc7)c56)n4)cc3)c2)cc1 ; c1ccc(-c2ccc(-c3nc(-c4ccc(-c5ccccc5)cc4)nc(-c4cccc5c6ccc7c8cc(-n9c%10ccccc%10c%10ccccc%109)ccc8oc7c6n(-c6ccccc6)c45)n3)cc2)cc1 ; c1ccc(-c2nc(-c3ccccc3)nc(-c3cccc4c5cc6c(cc5n(-c5ccccc5)c34)oc3ccc(-n4c5ccccc5c5ccccc54)cc36)n2)cc1 ; c1ccc(-c2nc(-c3ccccc3)nc(-c3cccc4c5ccc6oc7ccc(-n8c9ccccc9c9ccccc98)cc7c6c5n(-c5ccccc5)c34)n2)cc1]

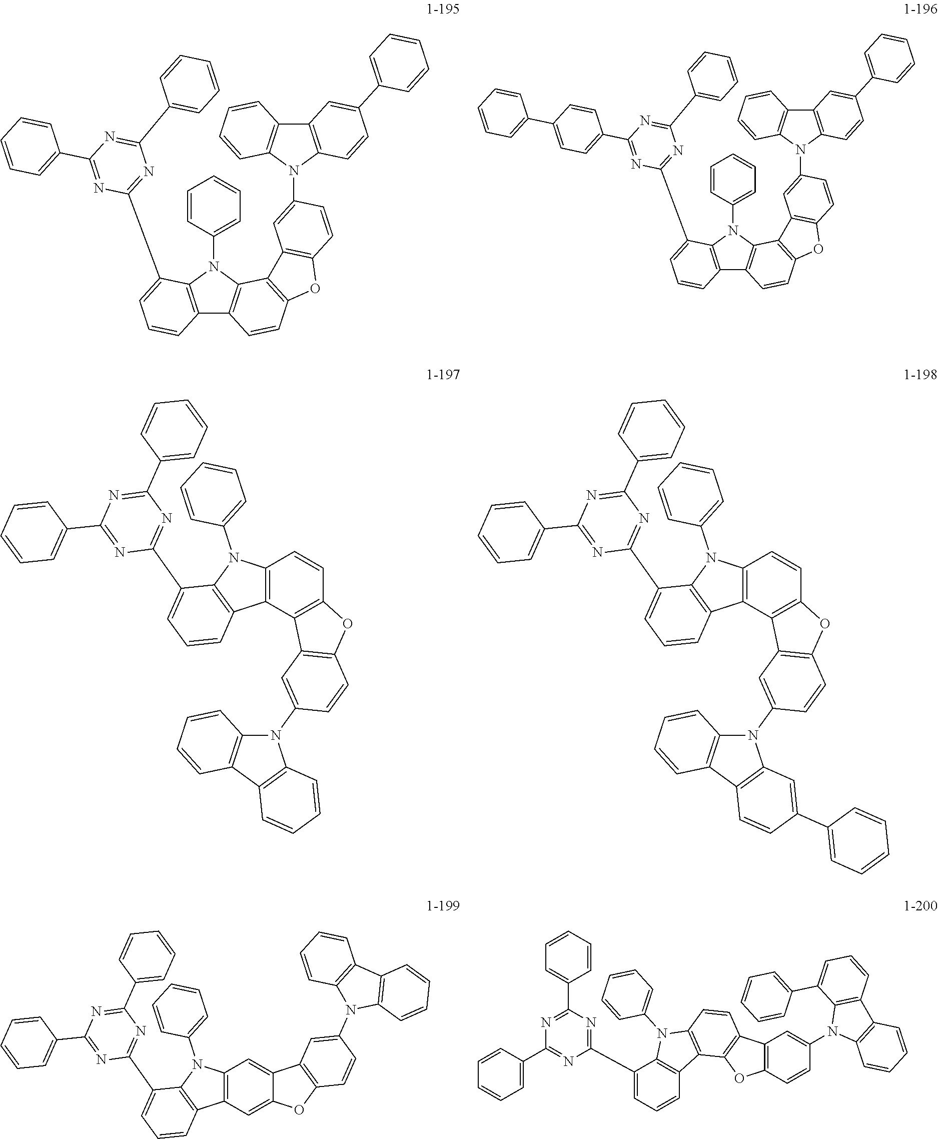

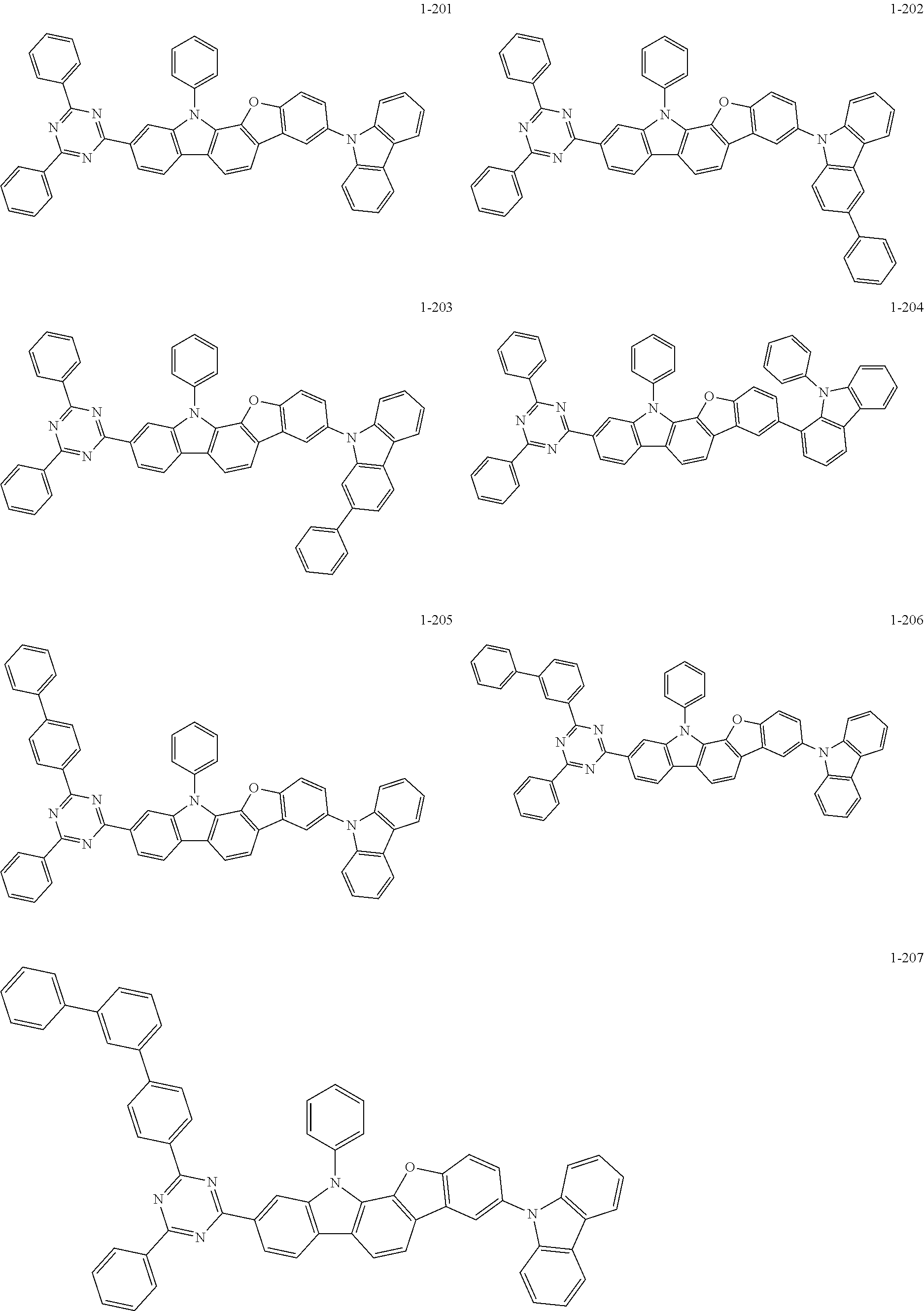

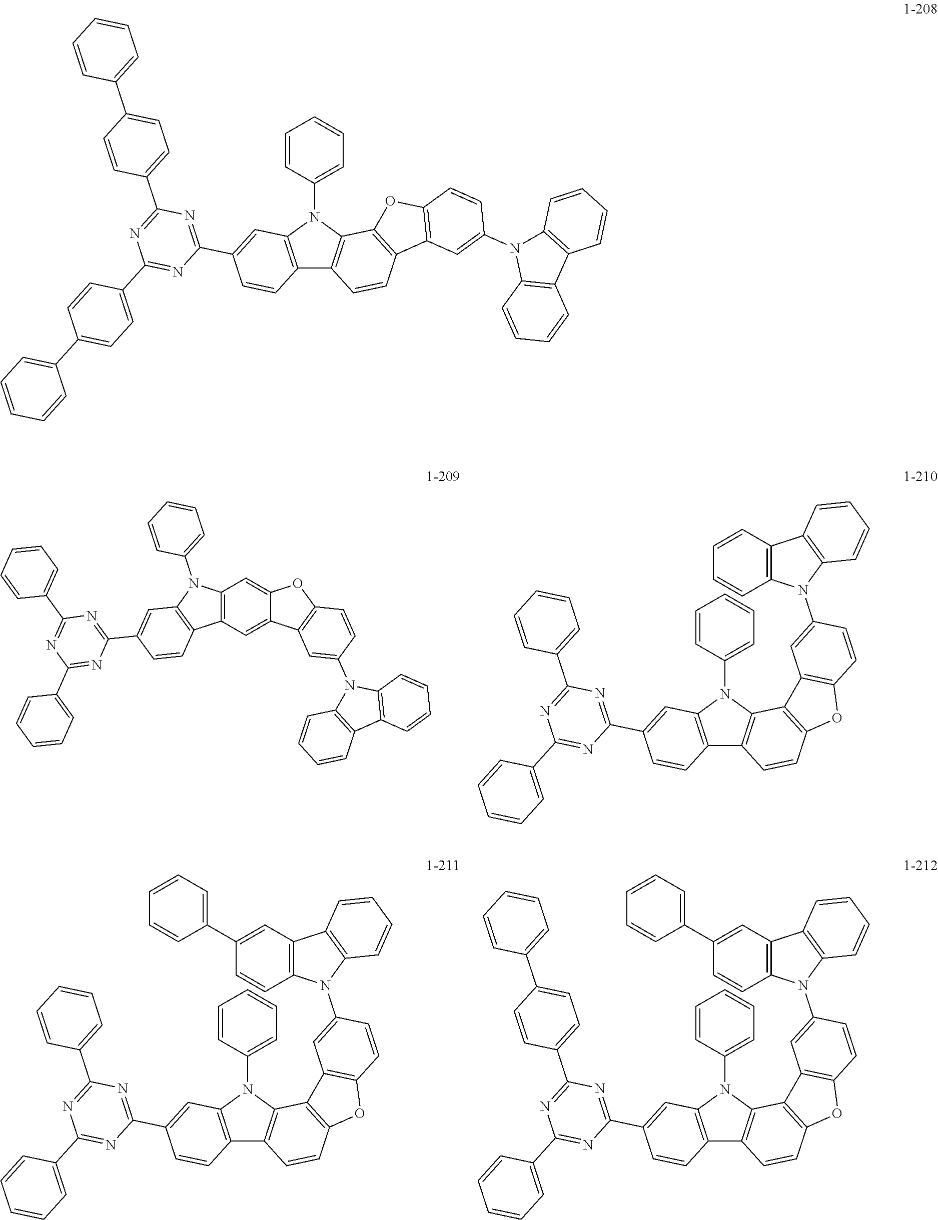

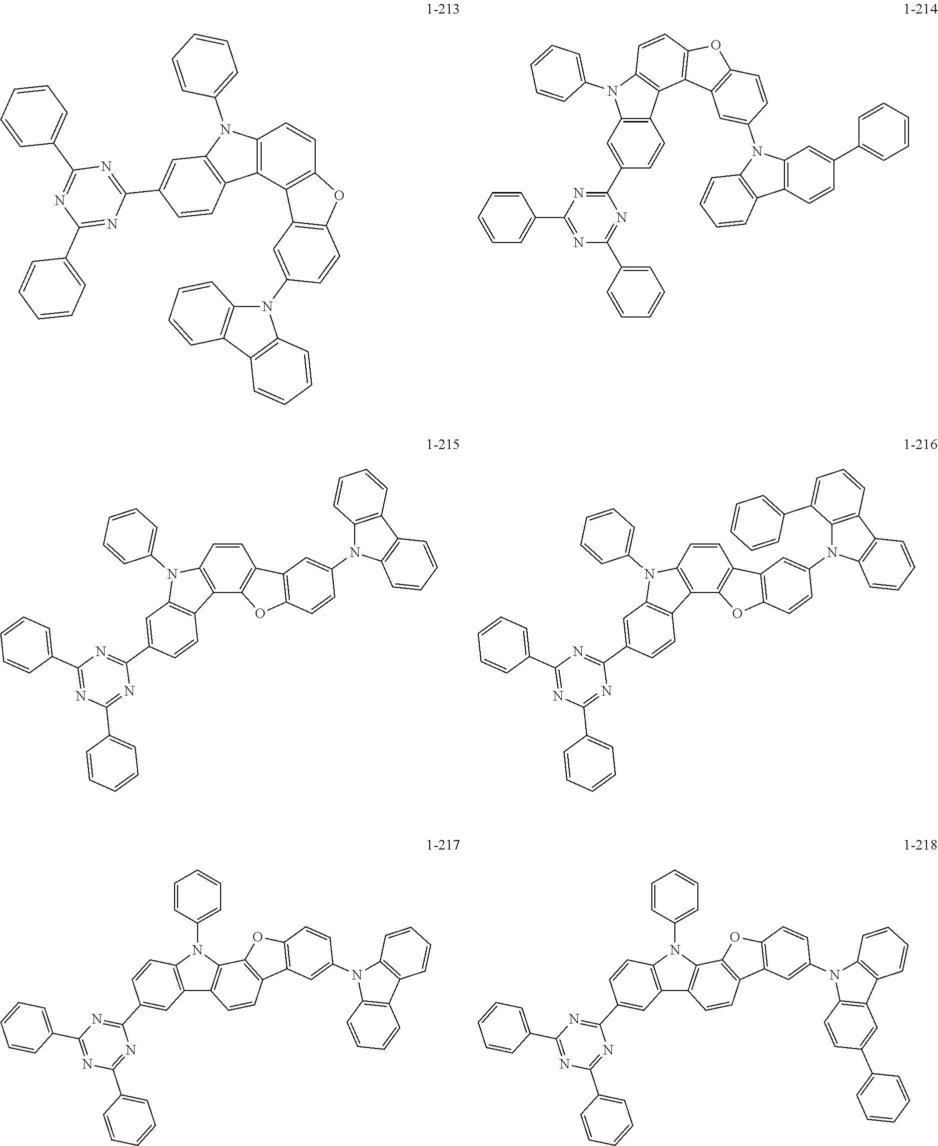

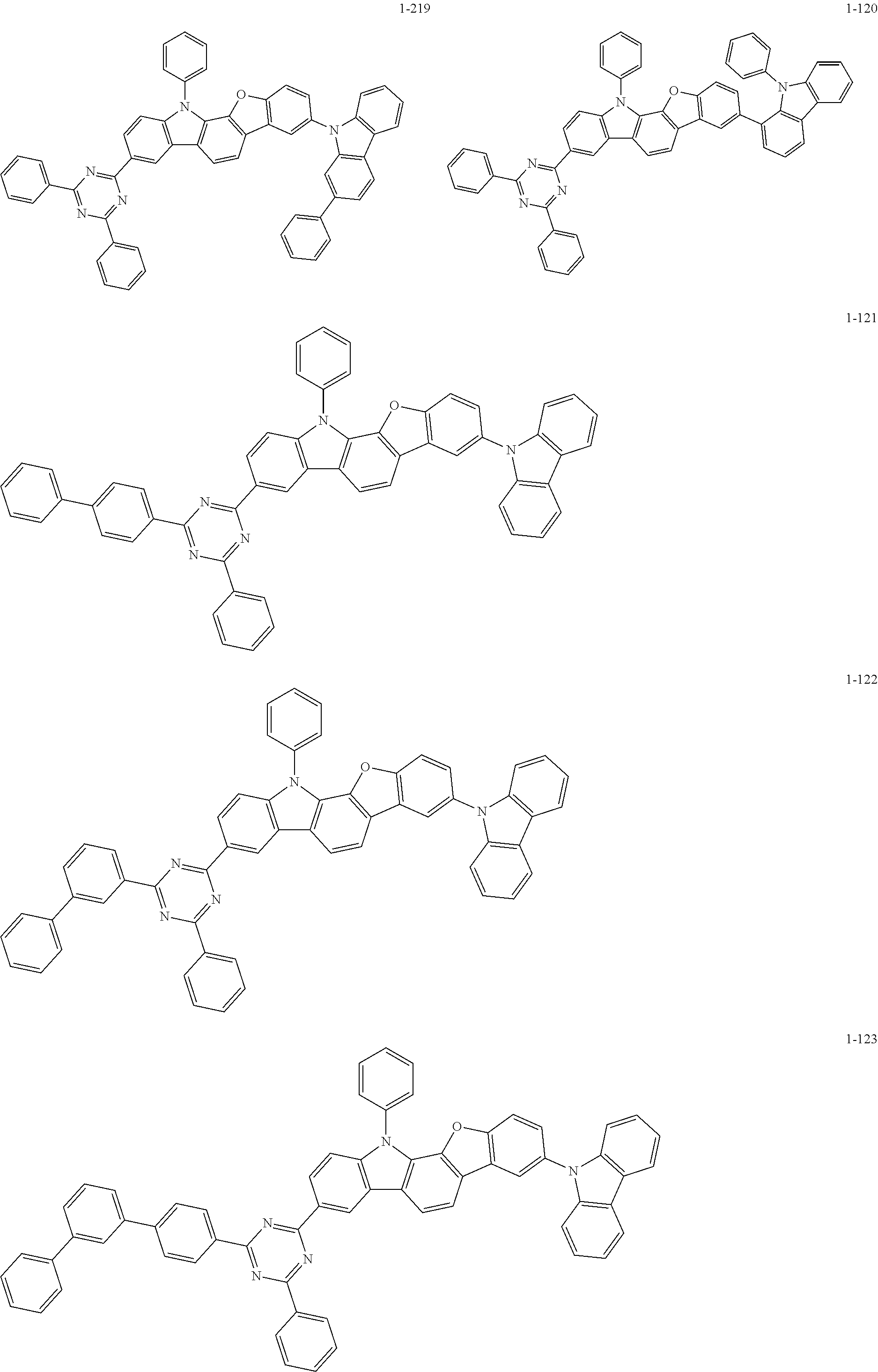

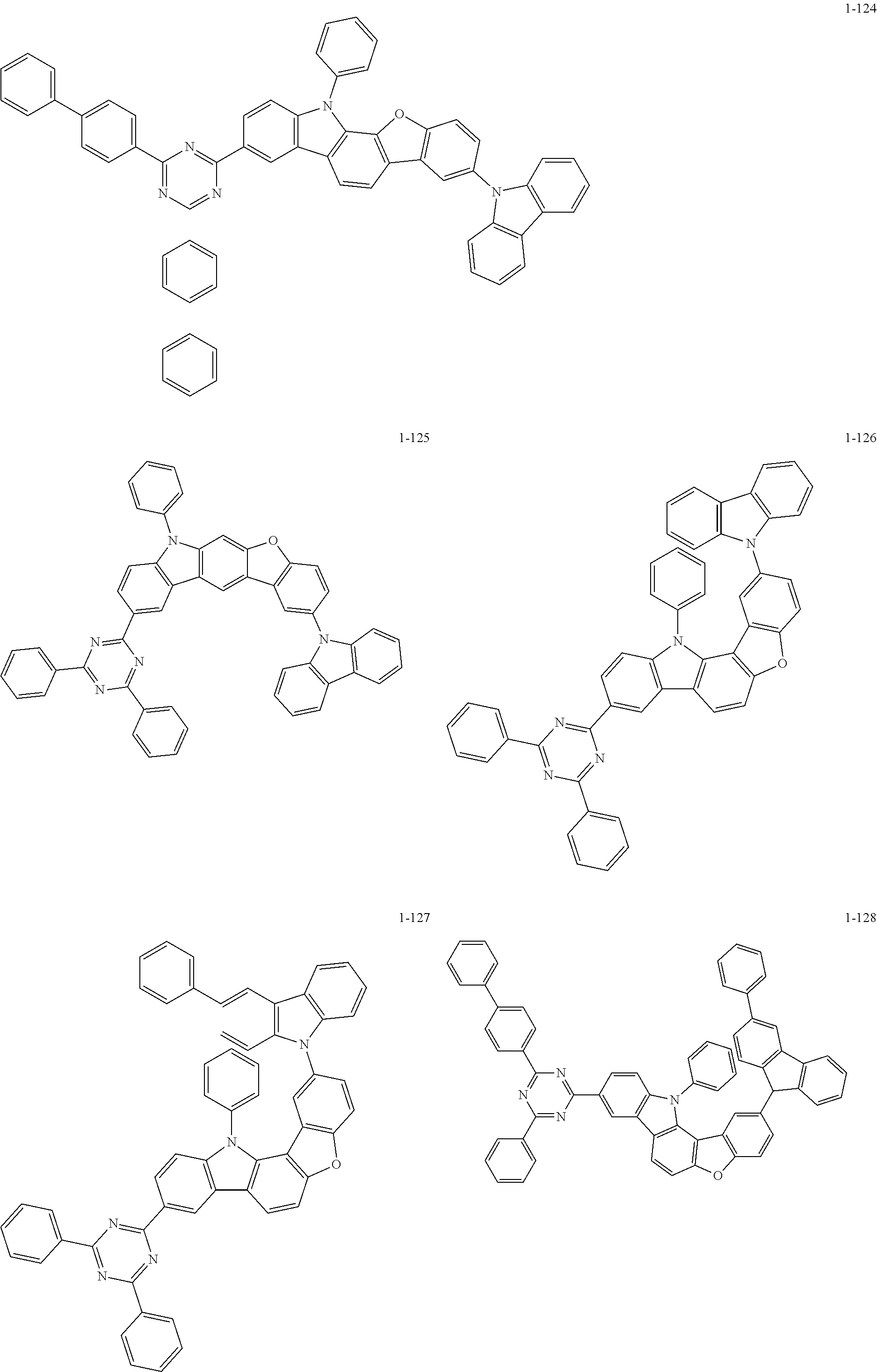

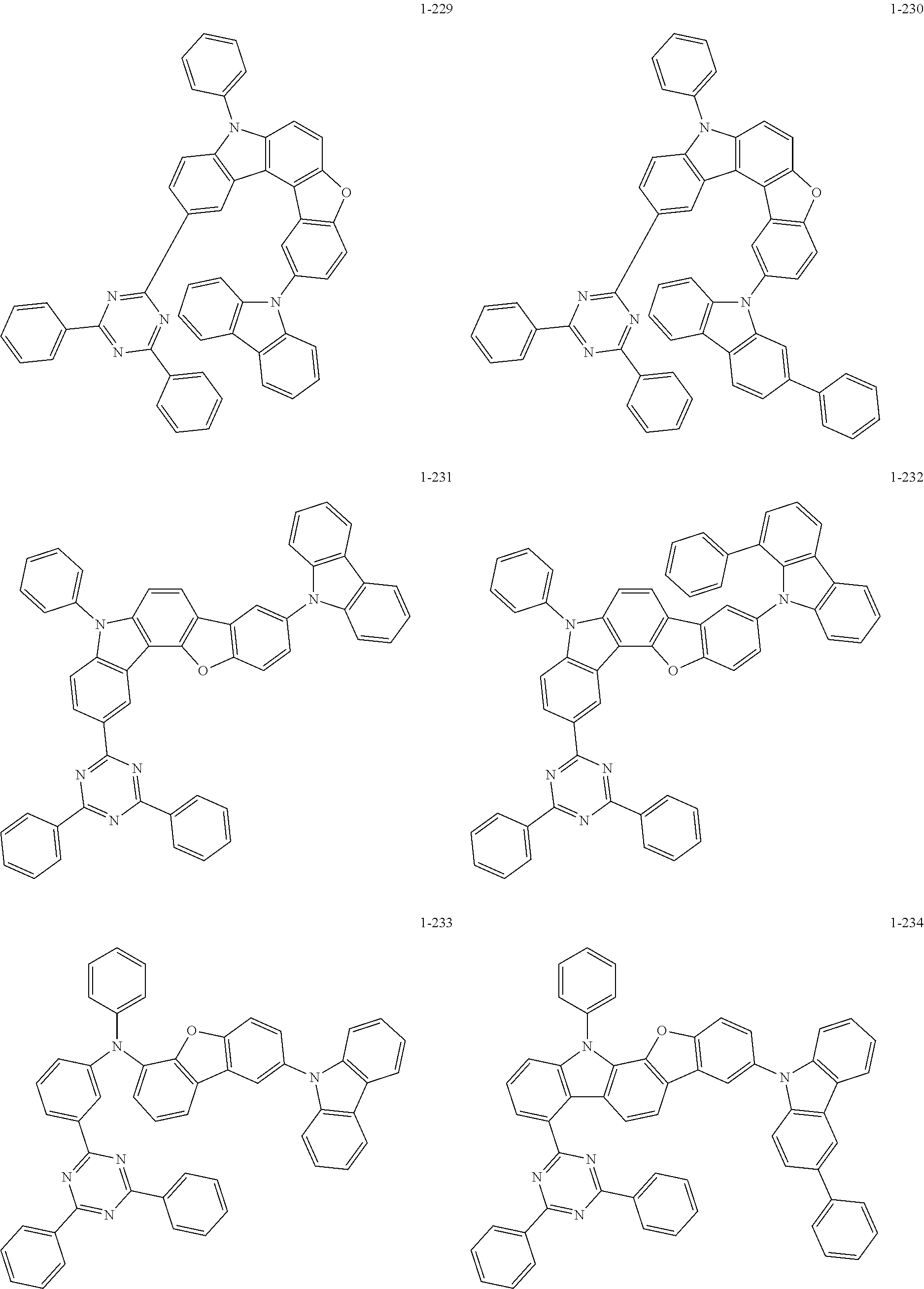

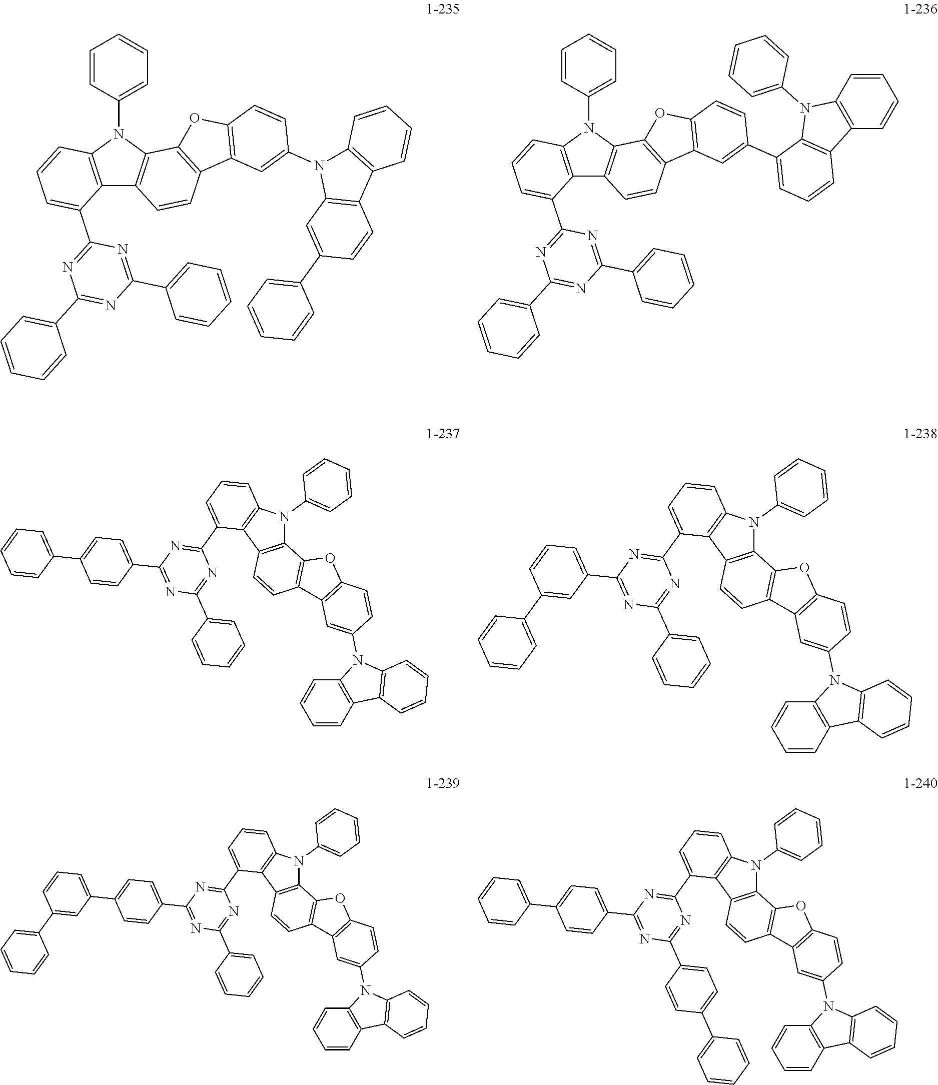

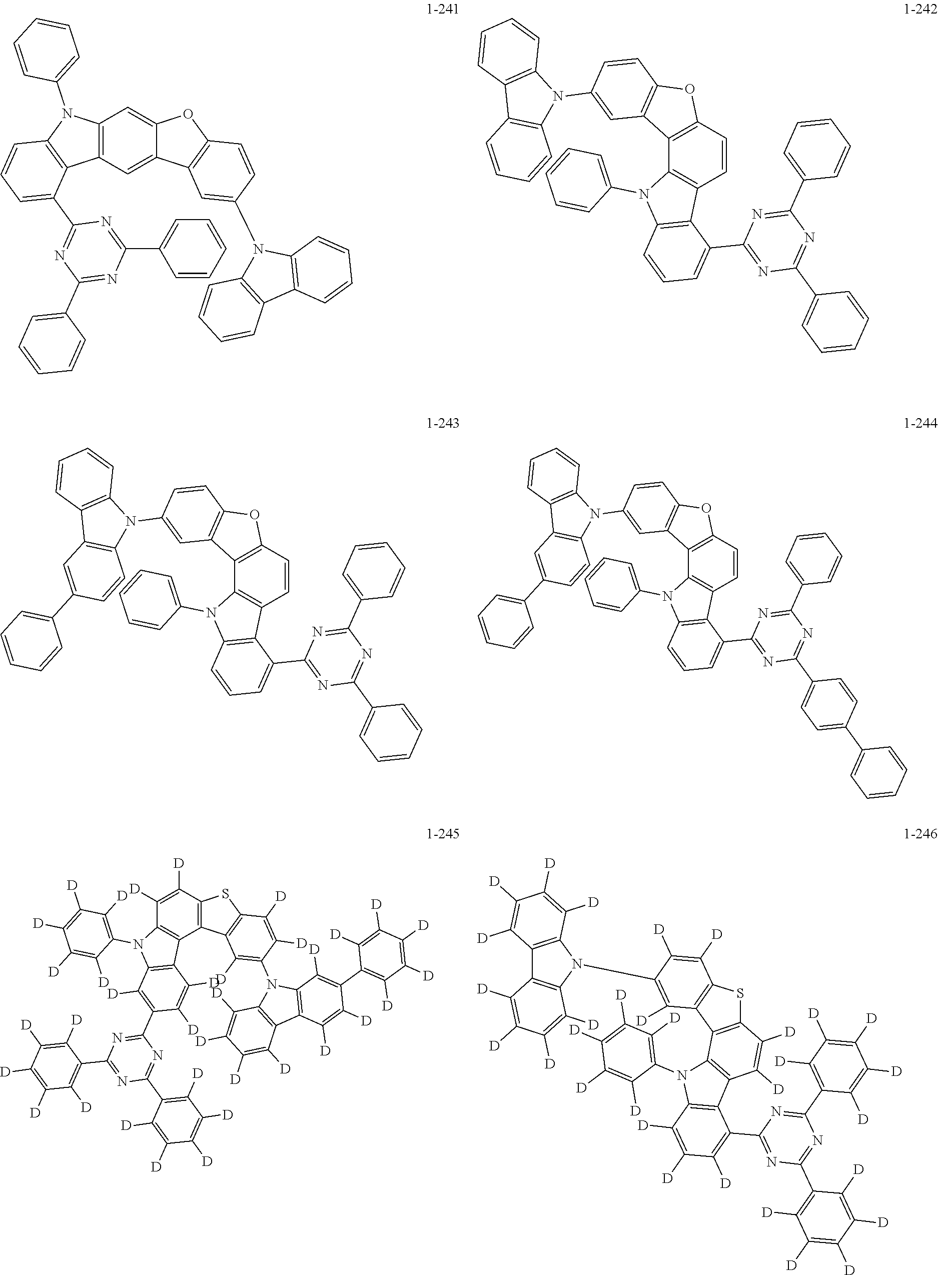

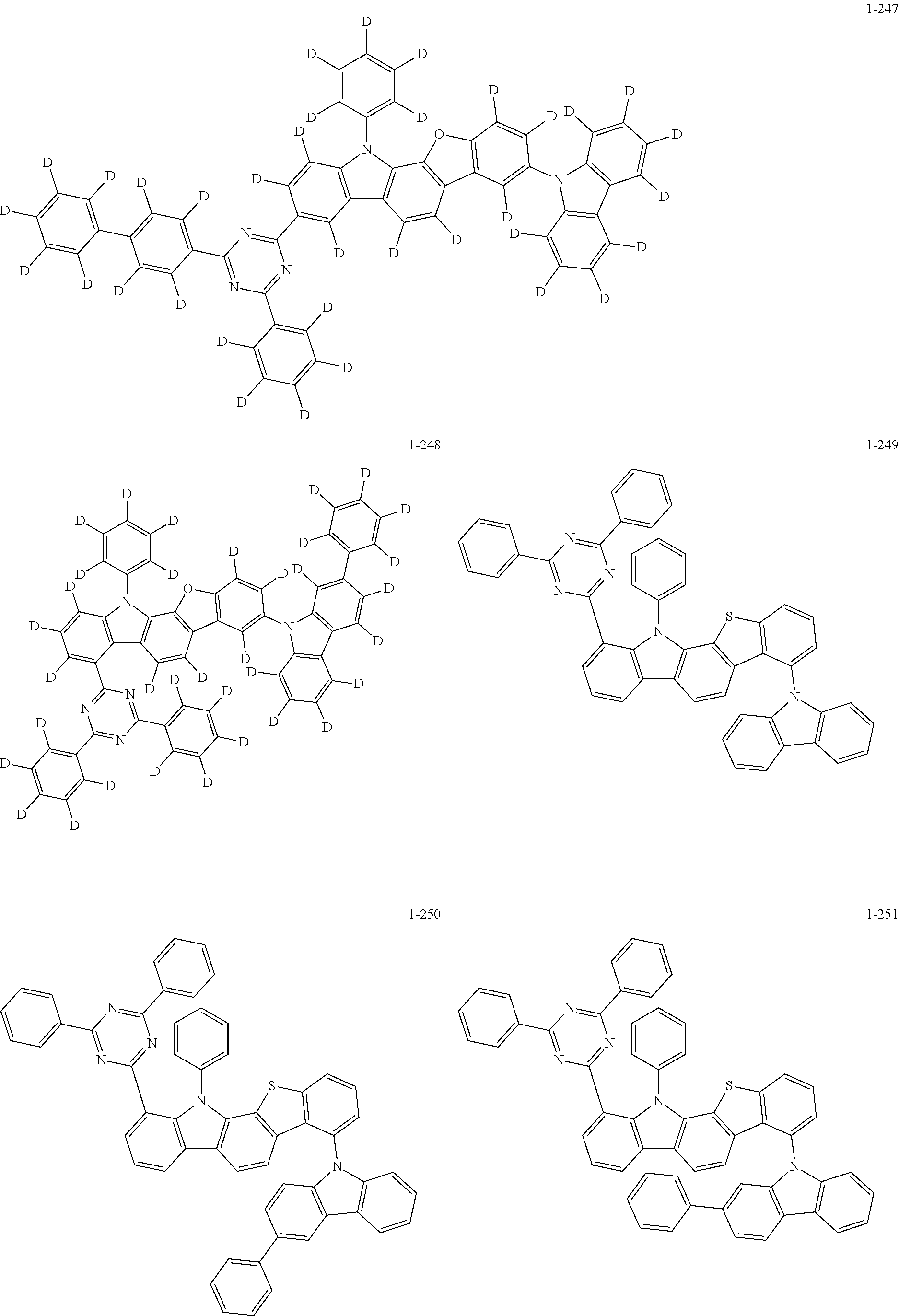

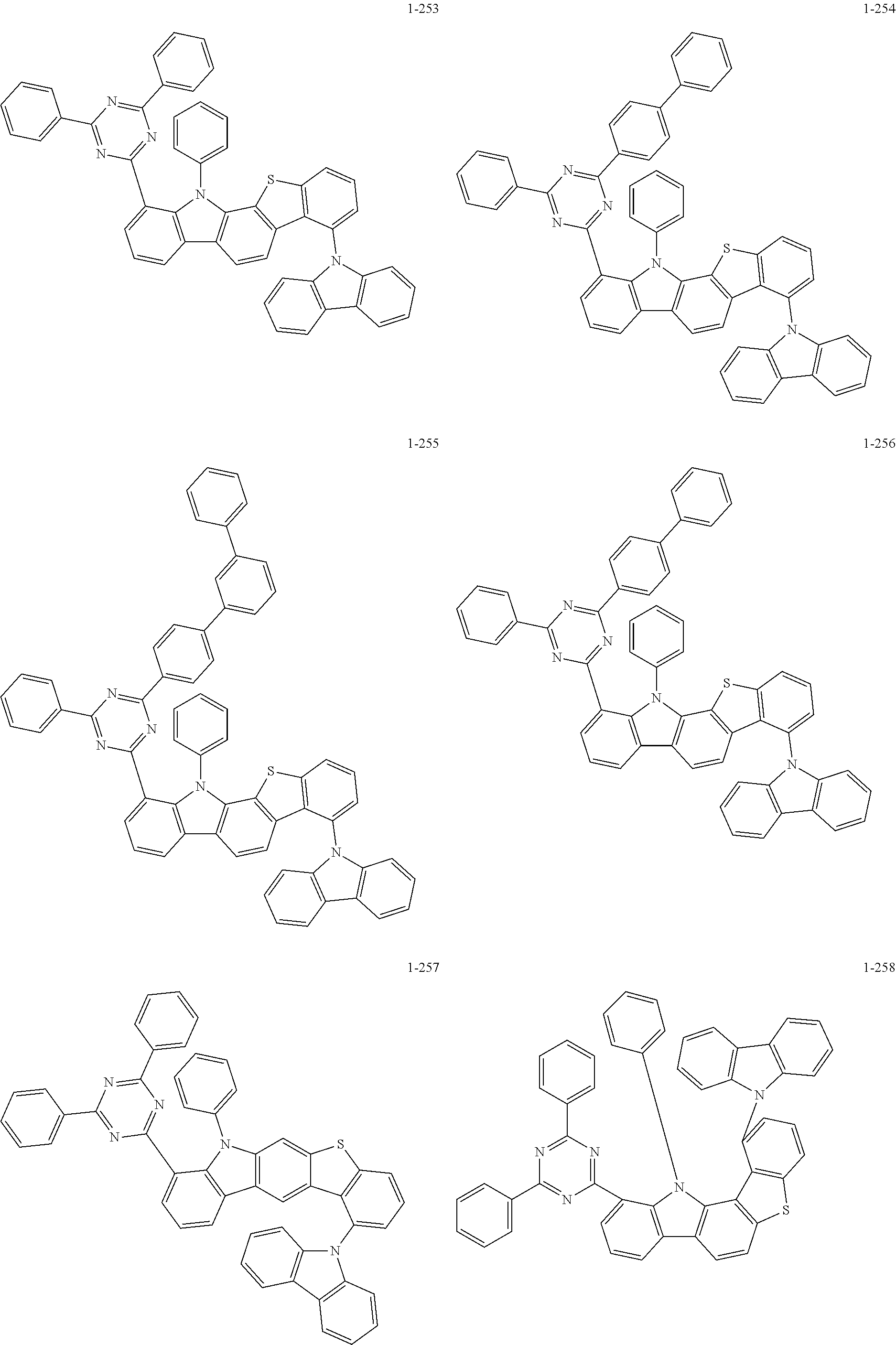

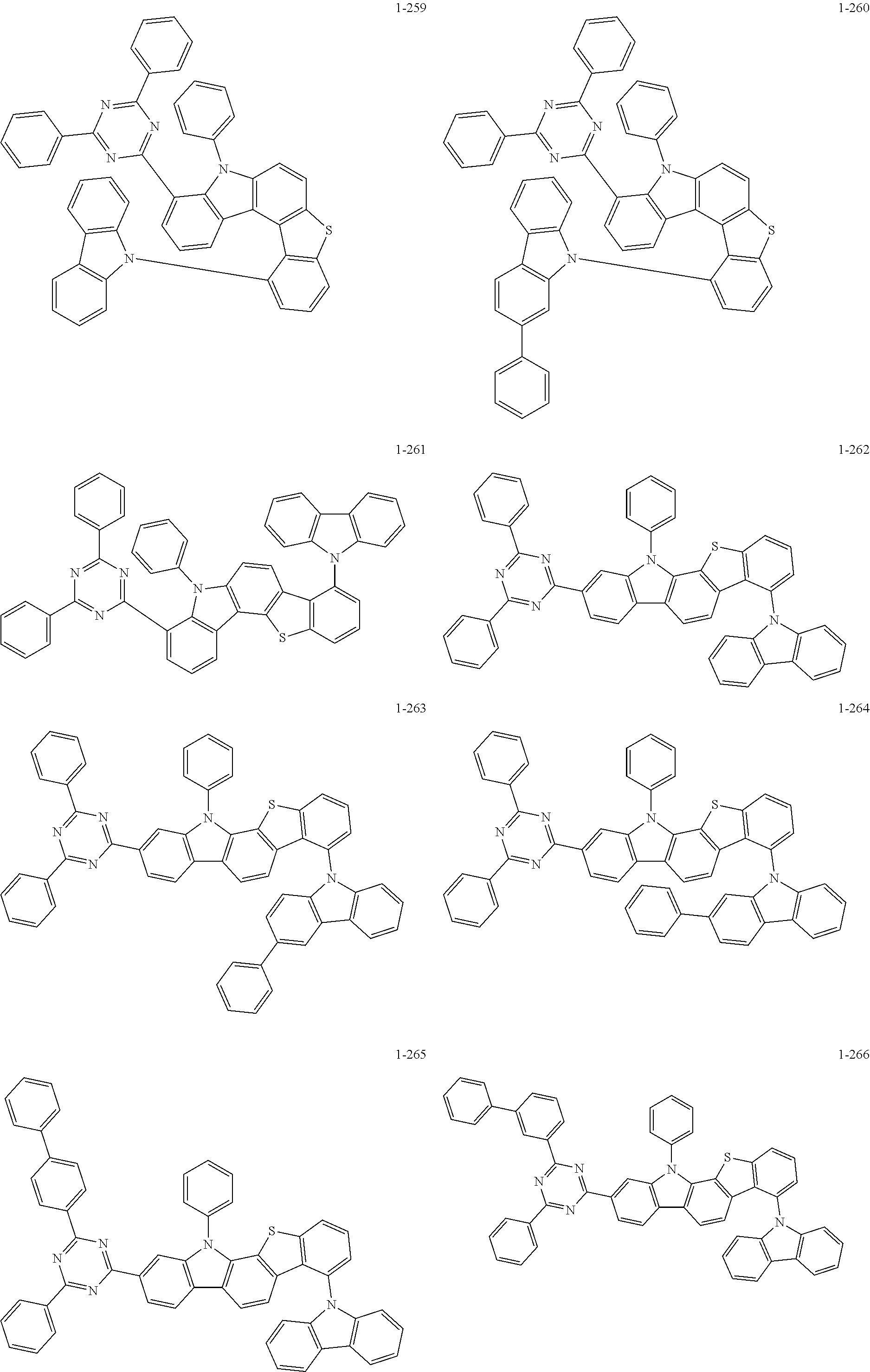

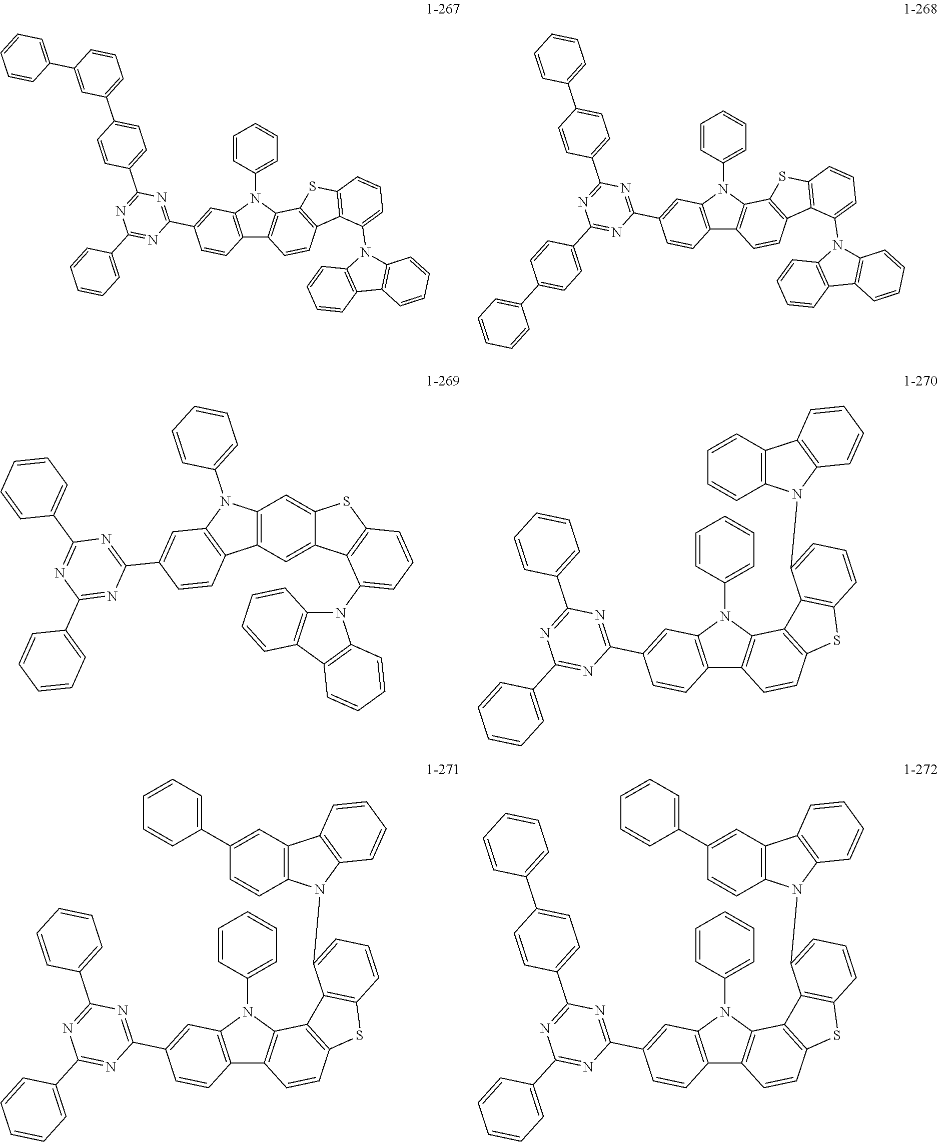

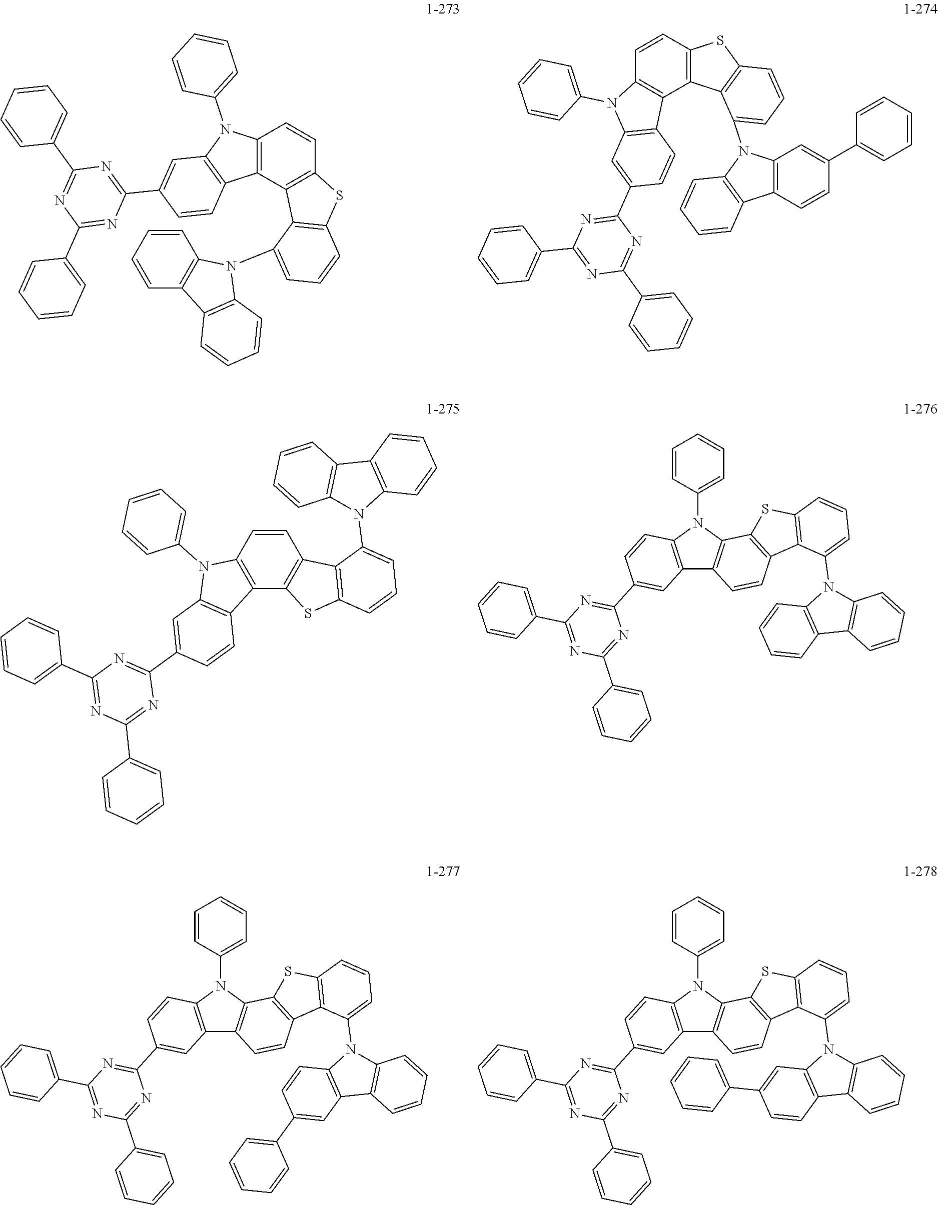

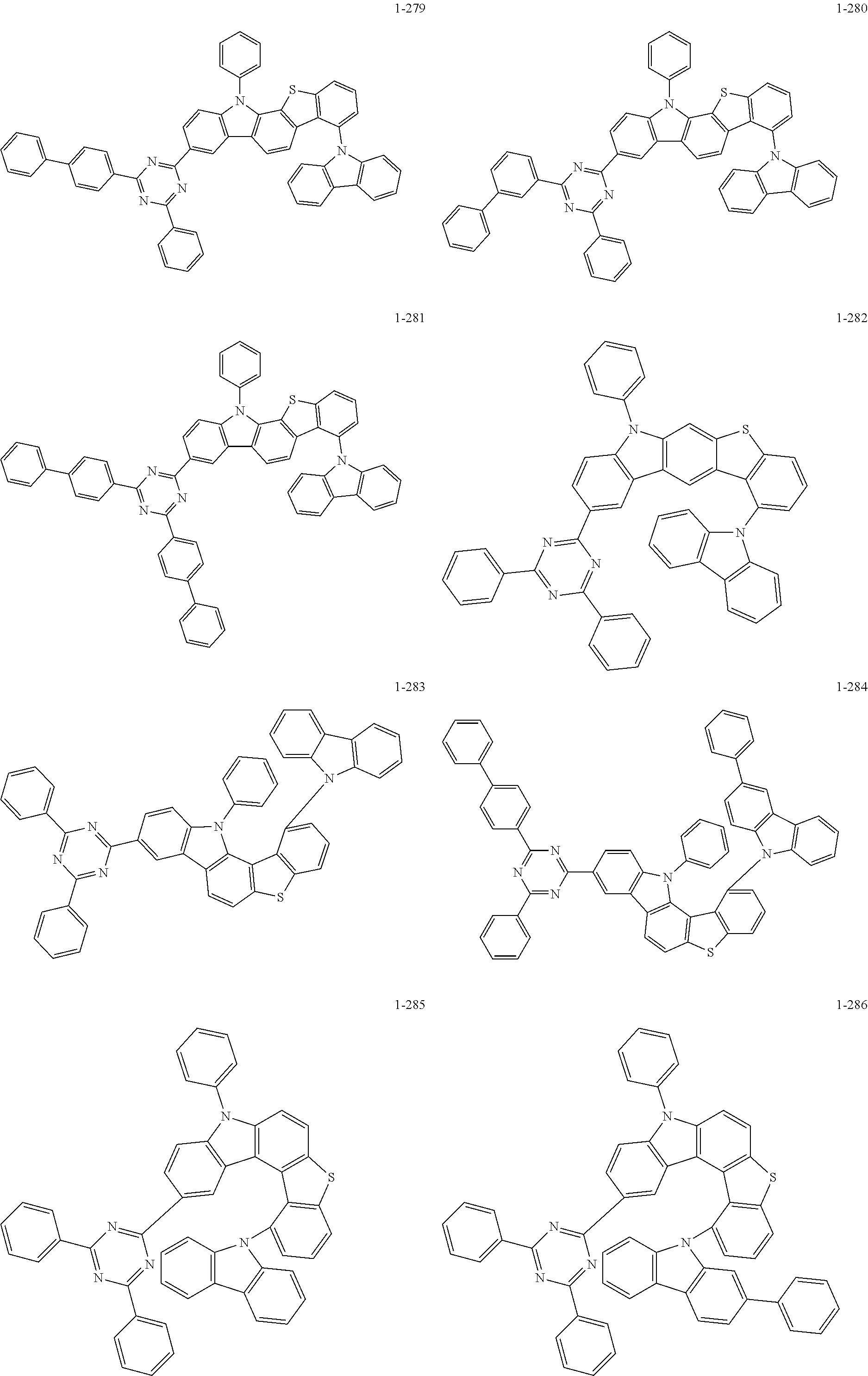

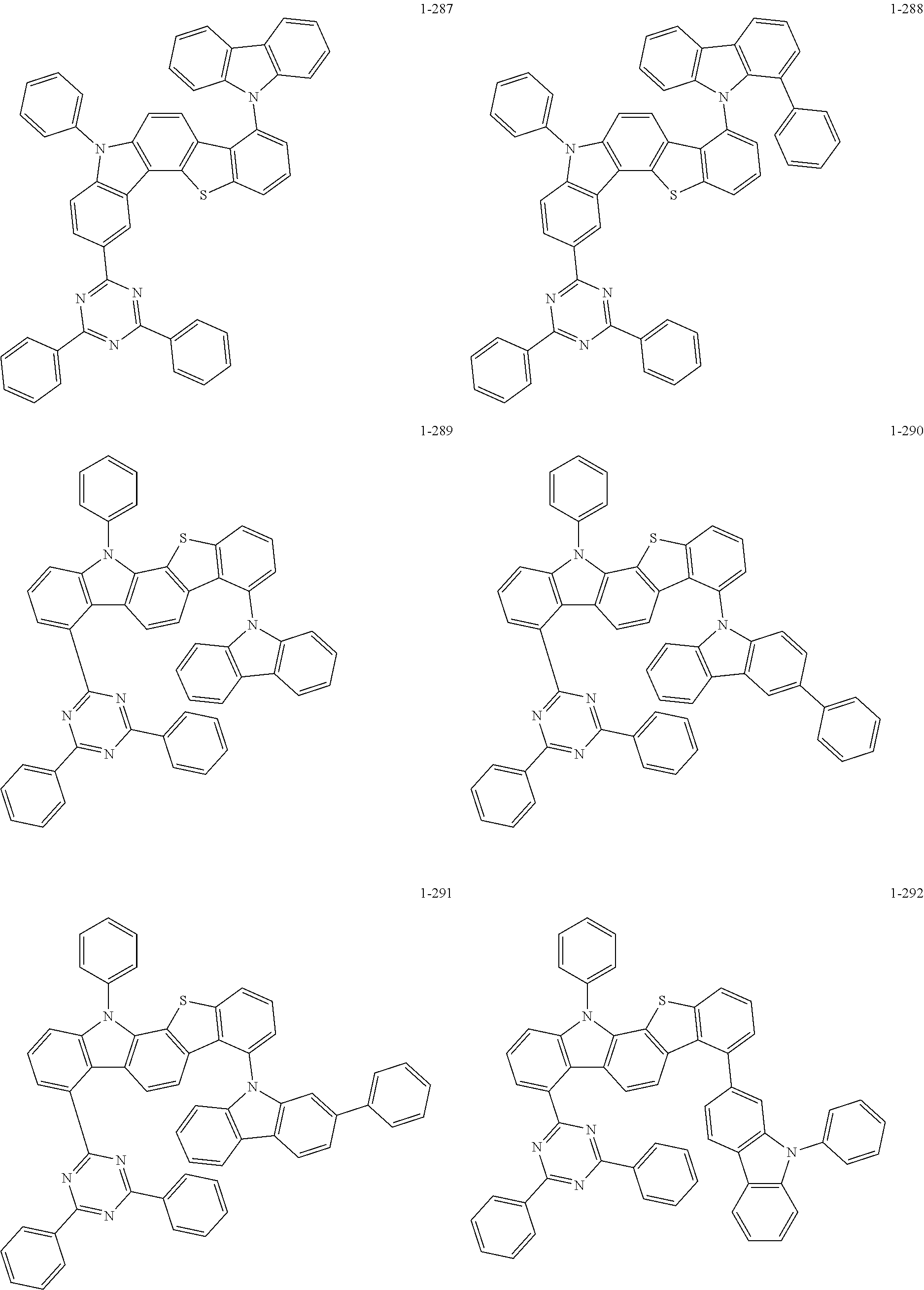

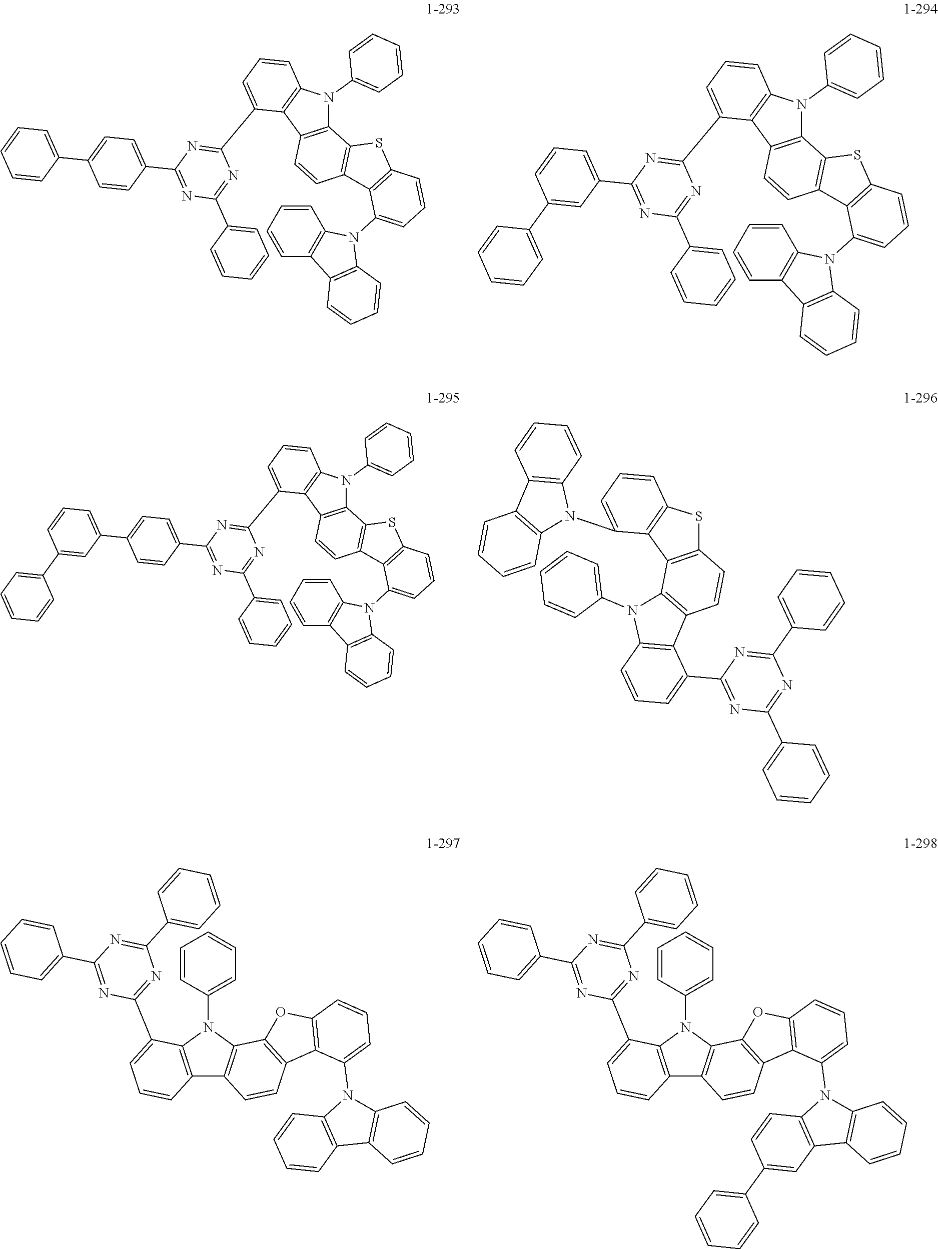

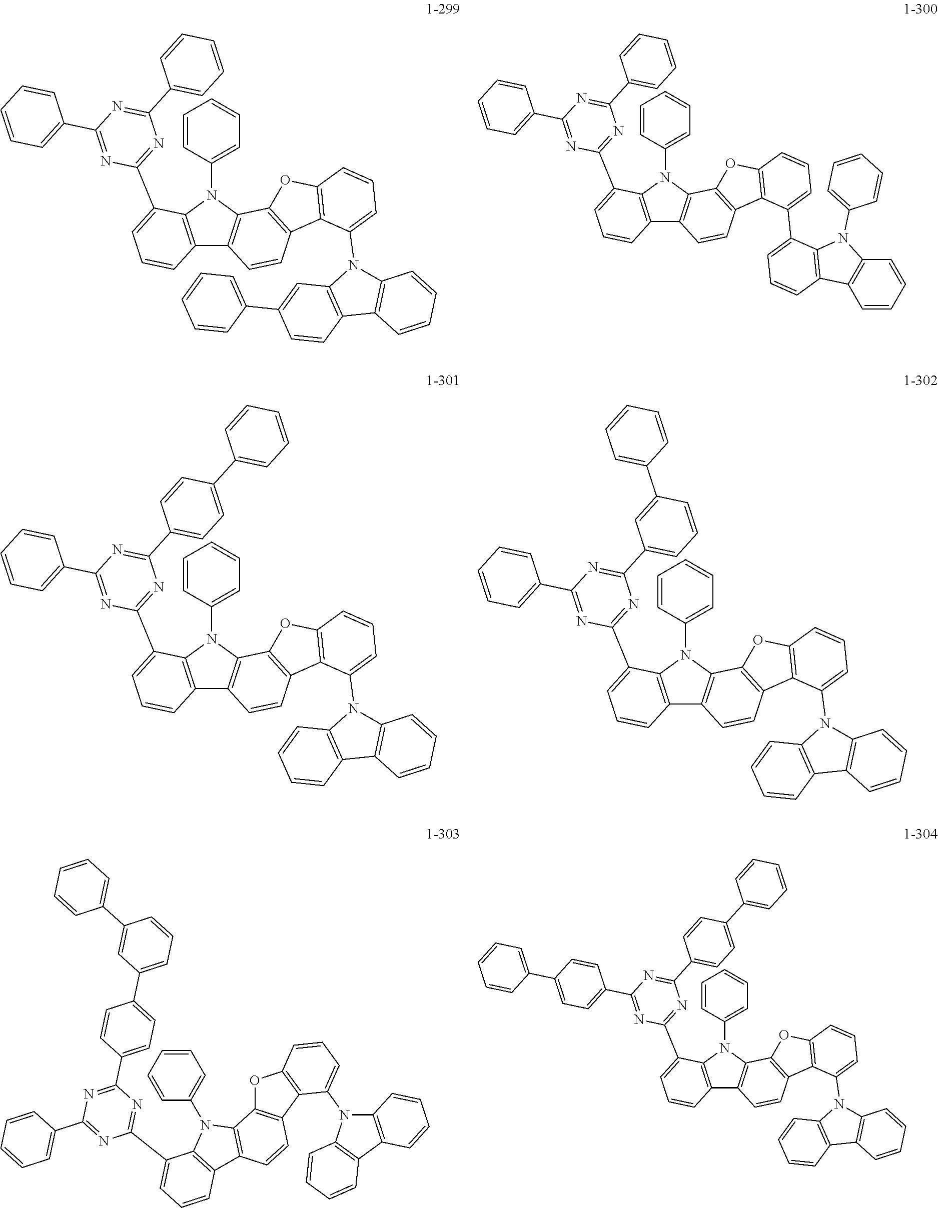

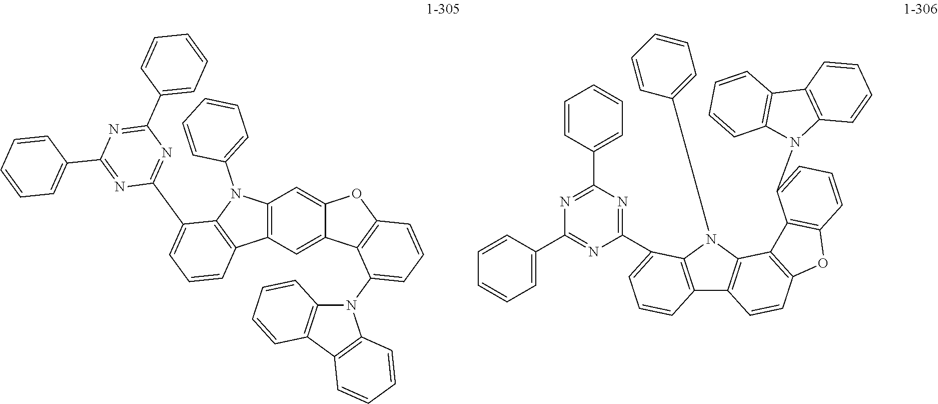 [SMILES: c1ccc(-c2nc(-c3ccccc3)nc(-c3cccc4c5cc6c(cc5n(-c5ccccc5)c34)oc3cccc(-n4c5ccccc5c5ccccc54)c36)n2)cc1 ; c1ccc(-c2nc(-c3ccccc3)nc(-c3cccc4c5ccc6oc7cccc(-n8c9ccccc9c9ccccc98)c7c6c5n(-c5ccccc5)c34)n2)cc1]

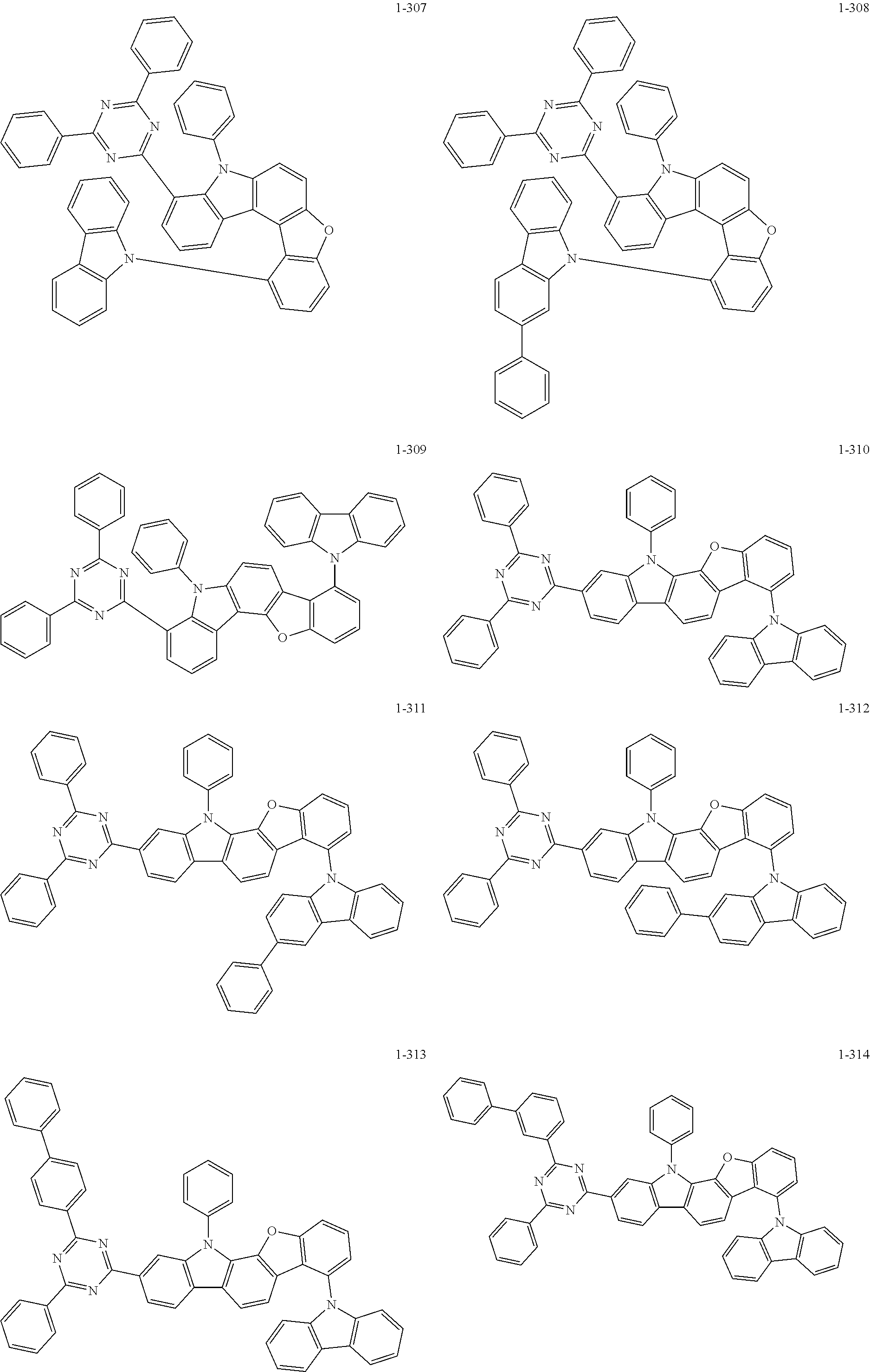

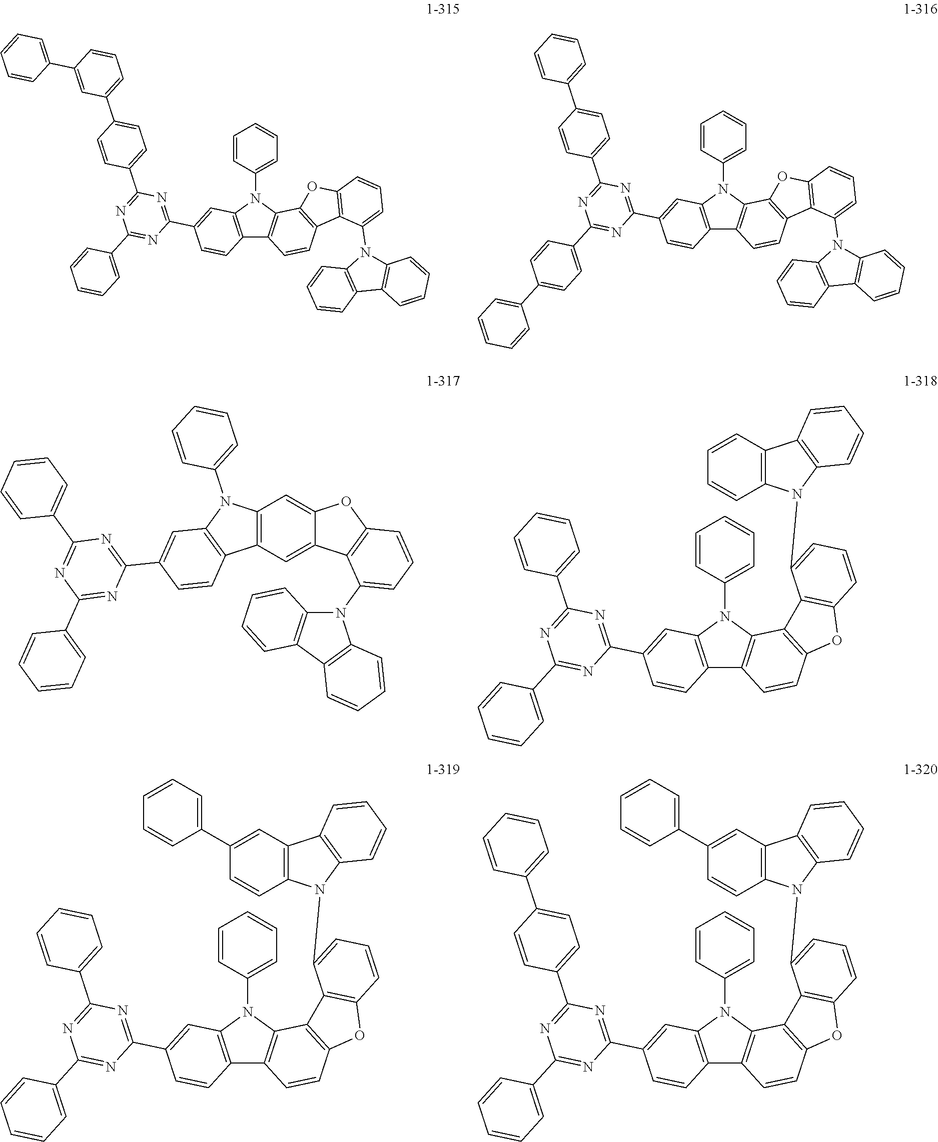

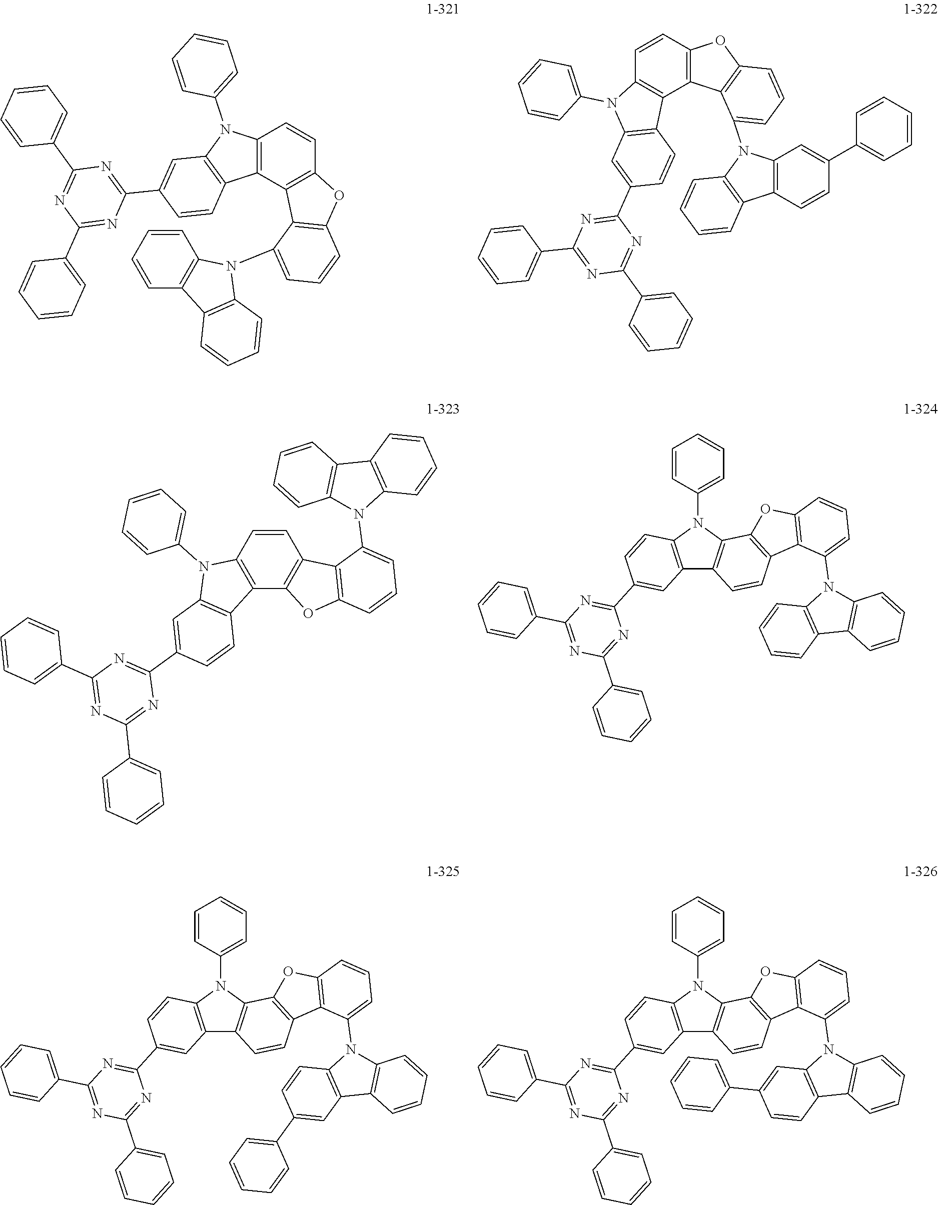

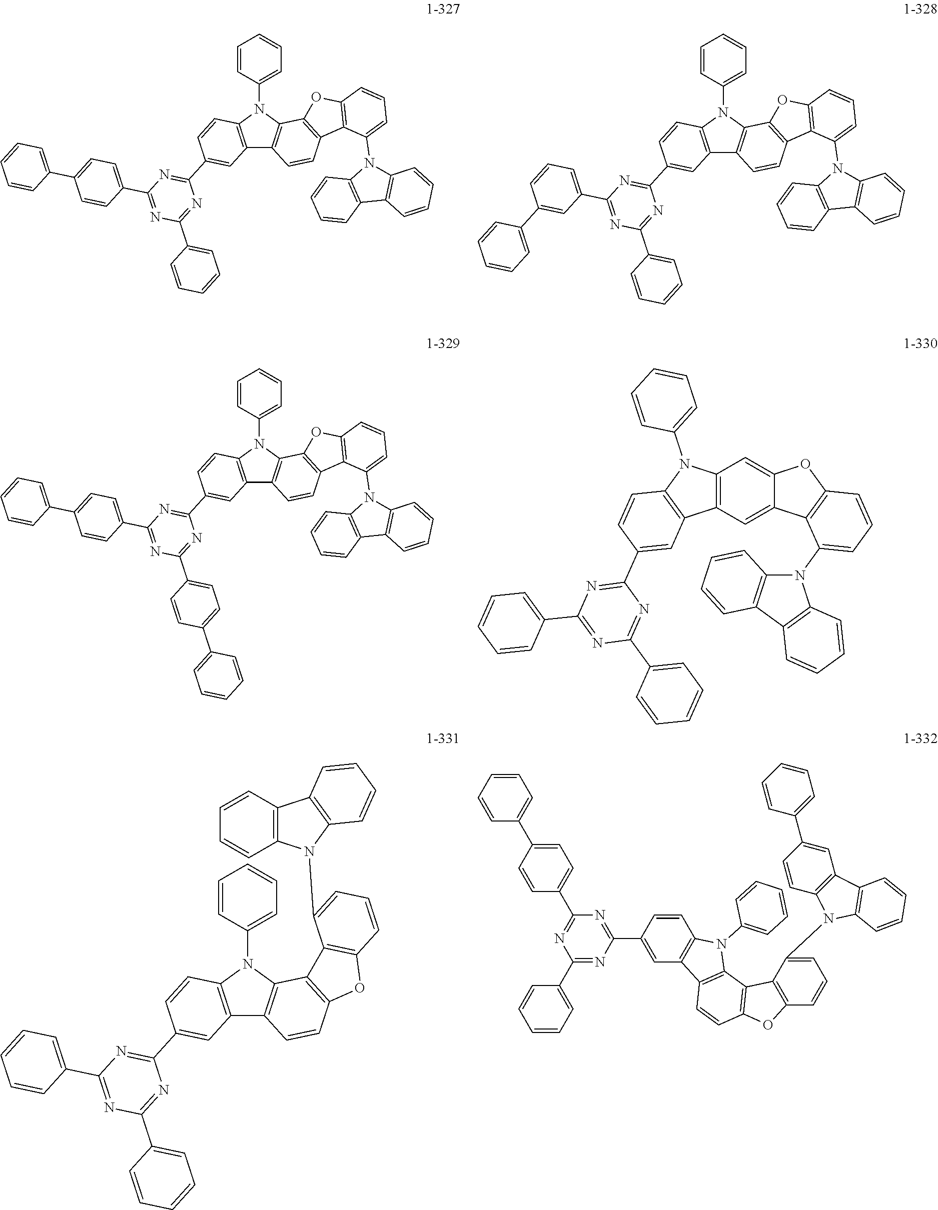

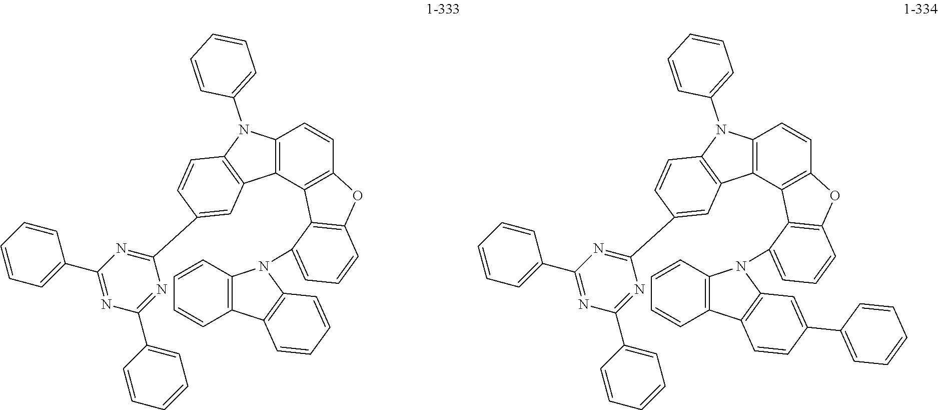 [SMILES: c1ccc(-c2nc(-c3ccccc3)nc(-c3ccc4c(c3)c3c5c(ccc3n4-c3ccccc3)oc3cccc(-n4c6ccccc6c6ccccc64)c35)n2)cc1 ; c1ccc(-c2ccc3c4ccccc4n(-c4cccc5oc6ccc7c(c8cc(-c9nc(-c%10ccccc%10)nc(-c%10ccccc%10)n9)ccc8n7-c7ccccc7)c6c45)c3c2)cc1]

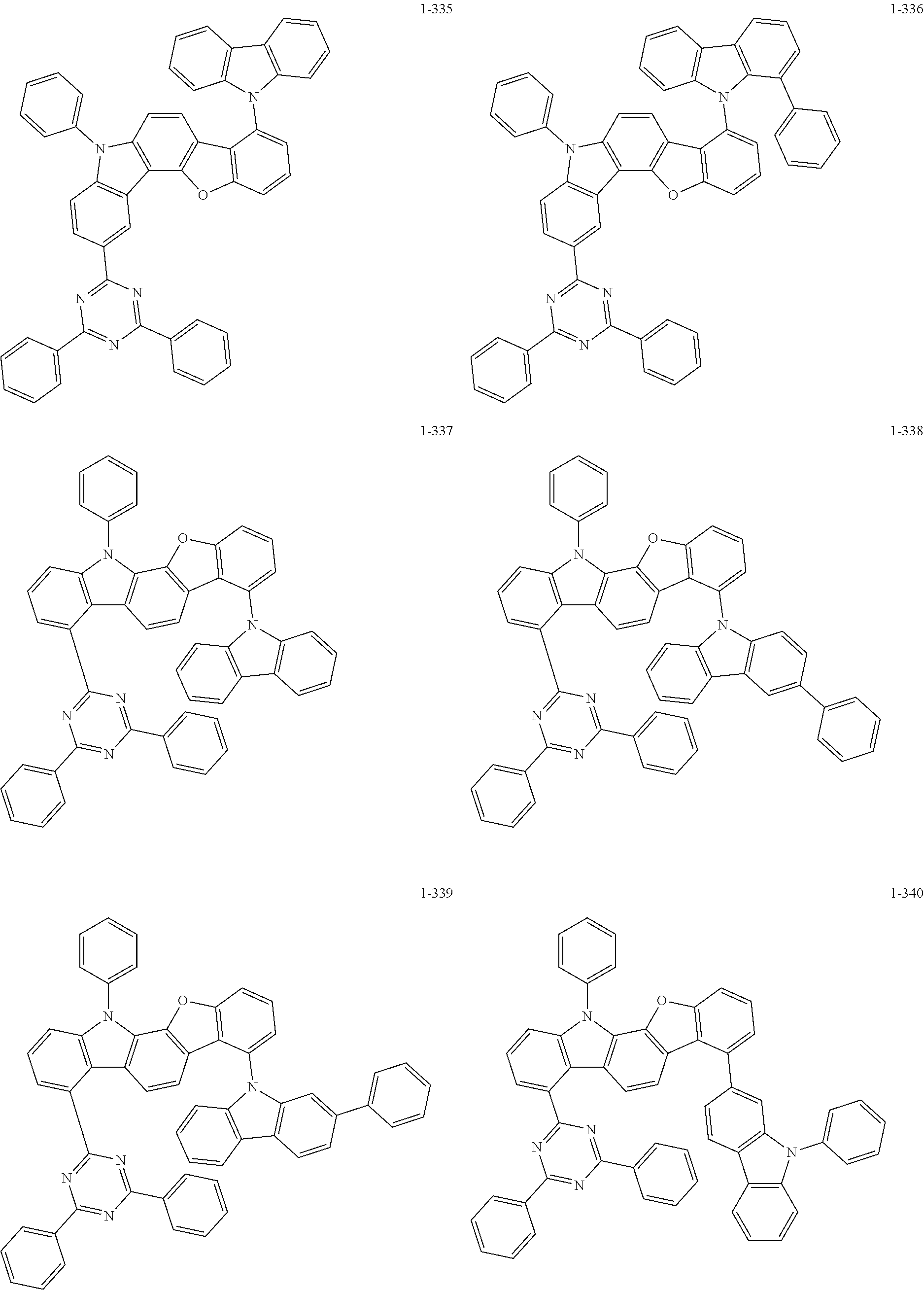

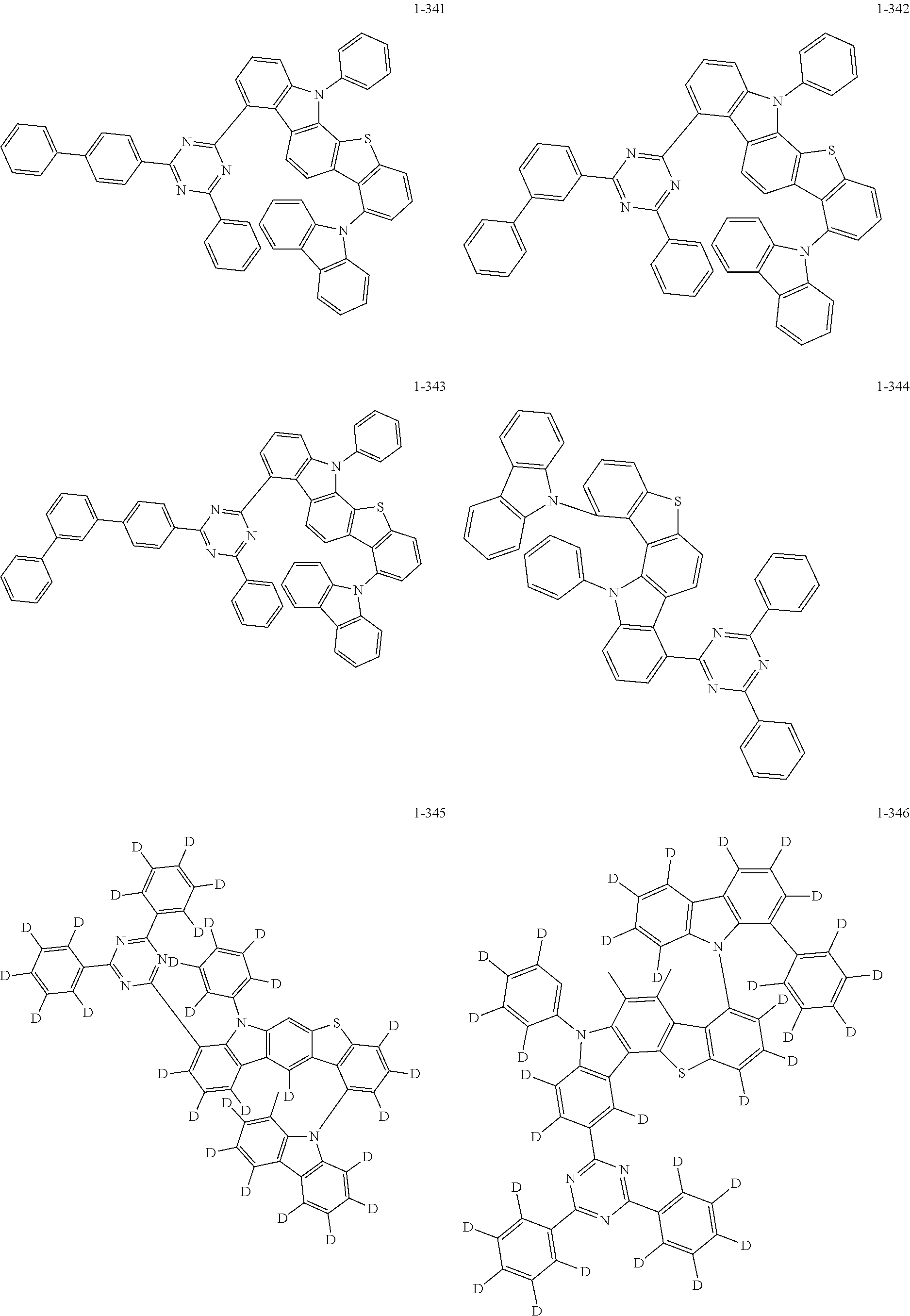

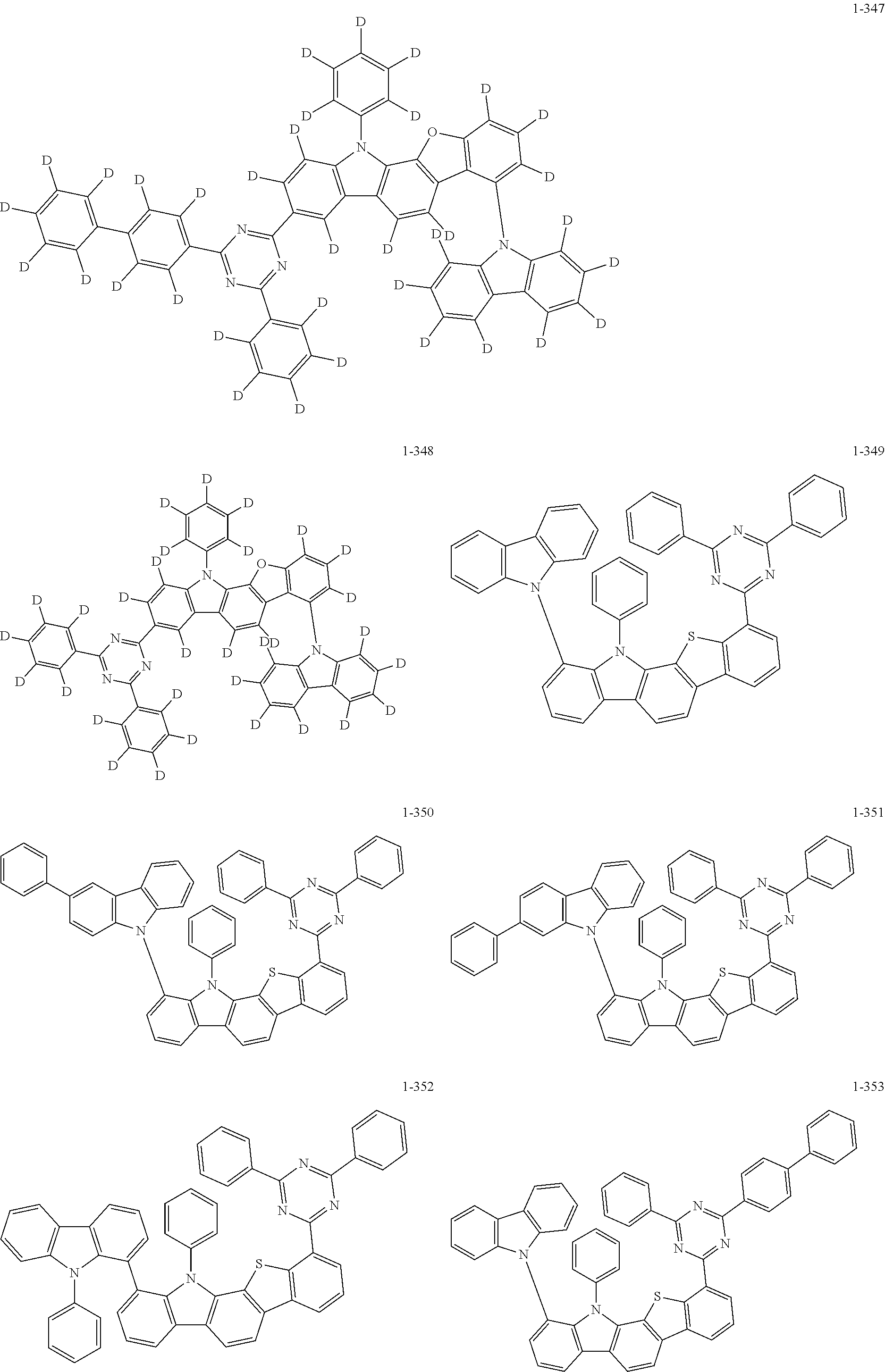

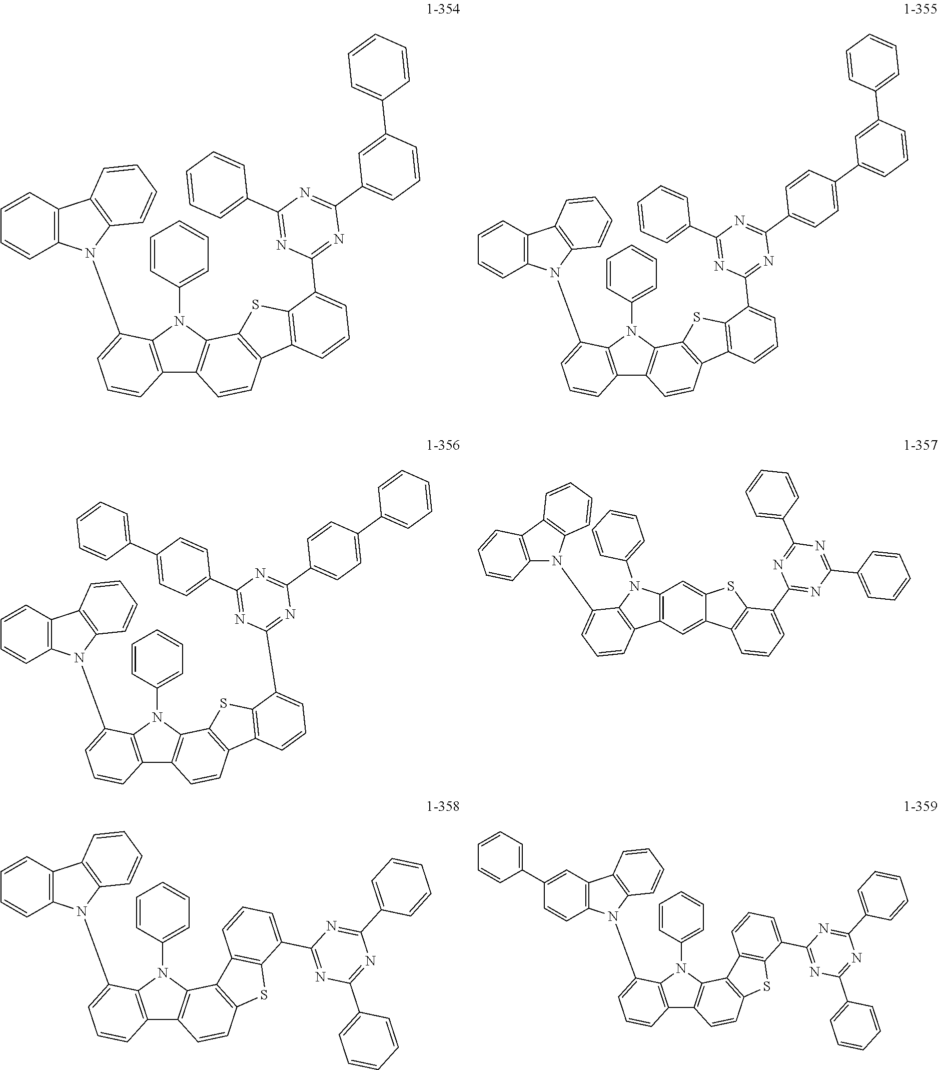

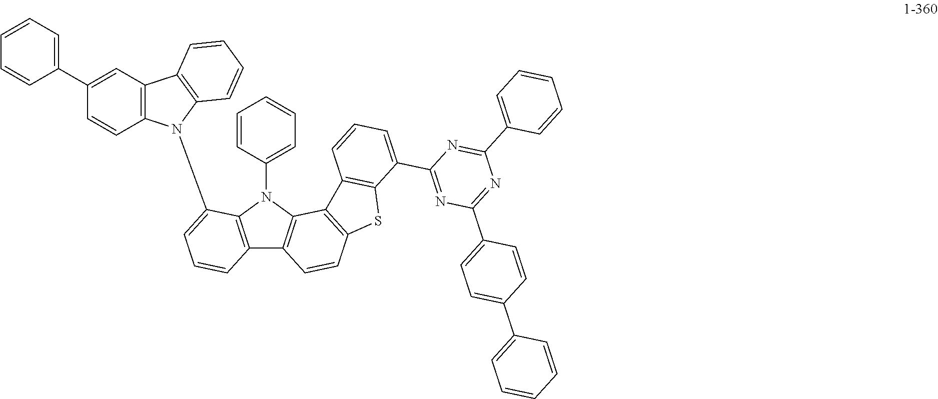 [SMILES: c1ccc(-c2ccc(-c3nc(-c4ccccc4)nc(-c4cccc5c4sc4ccc6c7cccc(-n8c9ccccc9c9cc(-c%10ccccc%10)ccc98)c7n(-c7ccccc7)c6c45)n3)cc2)cc1]

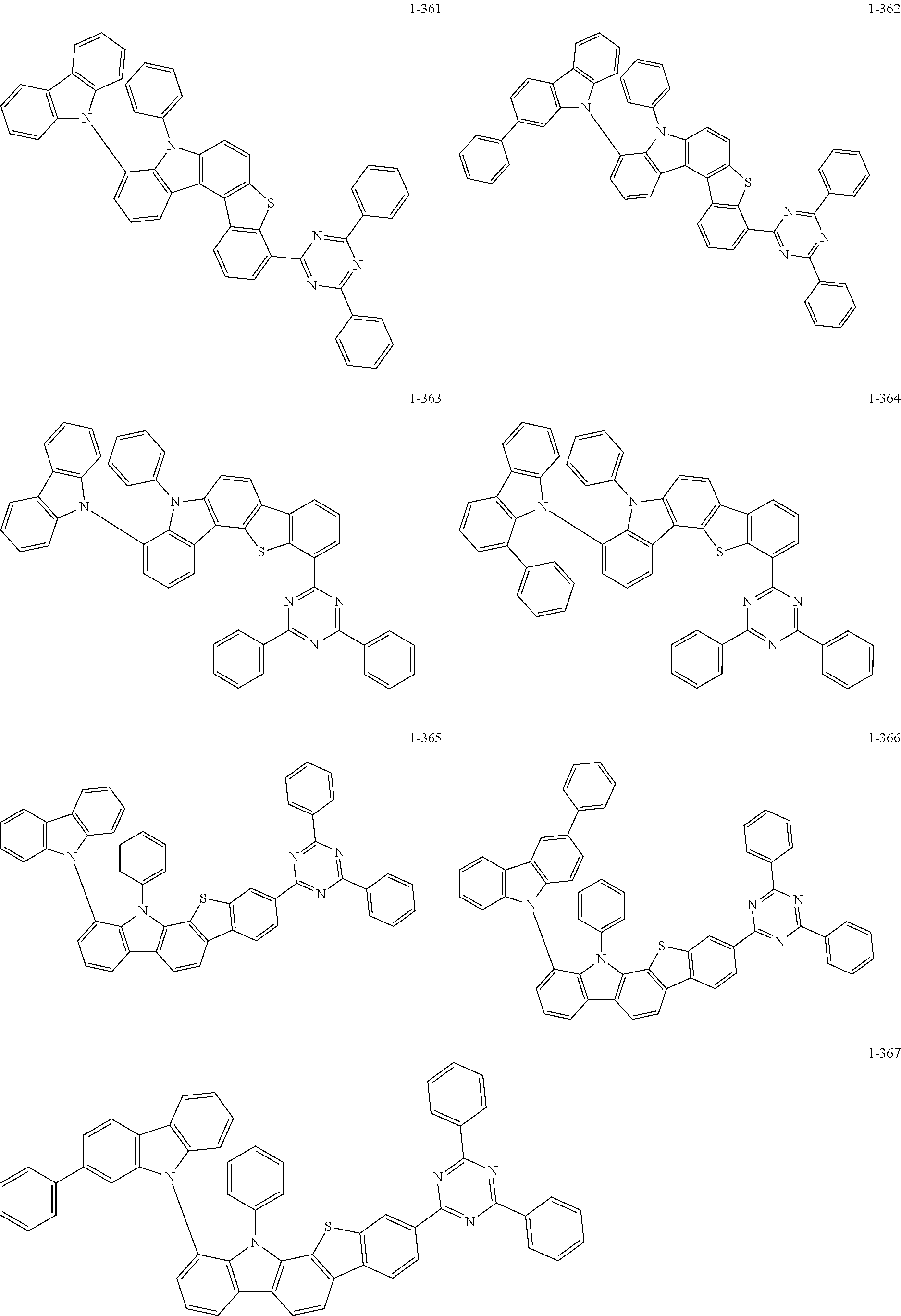

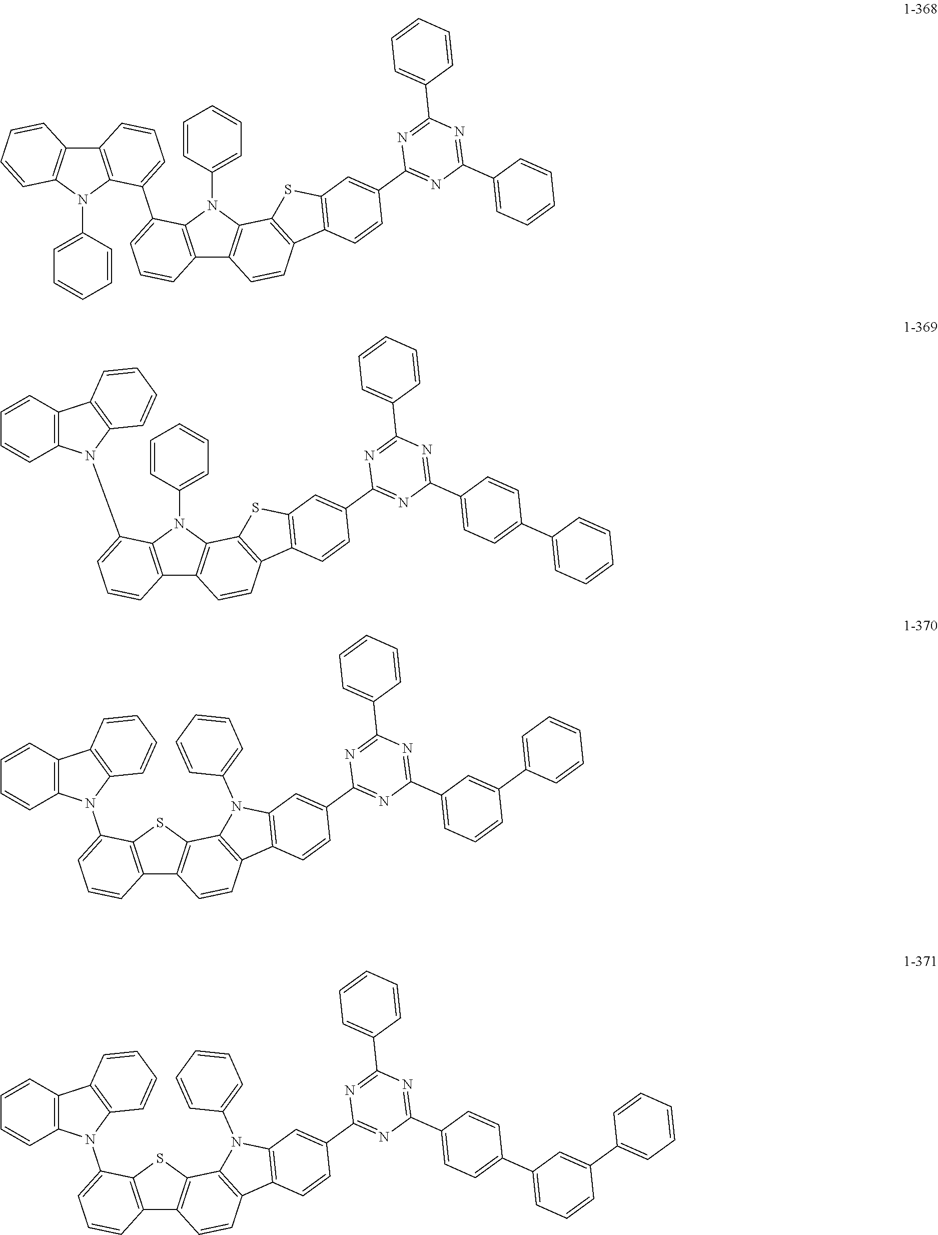 [SMILES: c1ccc(-c2nc(-c3ccccc3)nc(-c3ccc4c(c3)sc3c4ccc4c5cccc(-c6cccc7c8ccccc8n(-c8ccccc8)c67)c5n(-c5ccccc5)c43)n2)cc1 ; c1ccc(-c2ccc(-c3nc(-c4ccccc4)nc(-c4ccc5c(c4)sc4c5ccc5c6cccc(-n7c8ccccc8c8ccccc87)c6n(-c6ccccc6)c54)n3)cc2)cc1 ; c1ccc(-c2cccc(-c3nc(-c4ccccc4)nc(-c4ccc5c6ccc7c8cccc(-n9c%10ccccc%10c%10ccccc%109)c8sc7c6n(-c6ccccc6)c5c4)n3)c2)cc1 ; c1ccc(-c2cccc(-c3ccc(-c4nc(-c5ccccc5)nc(-c5ccc6c7ccc8c9cccc(-n%10c%11ccccc%11c%11ccccc%11%10)c9sc8c7n(-c7ccccc7)c6c5)n4)cc3)c2)cc1]

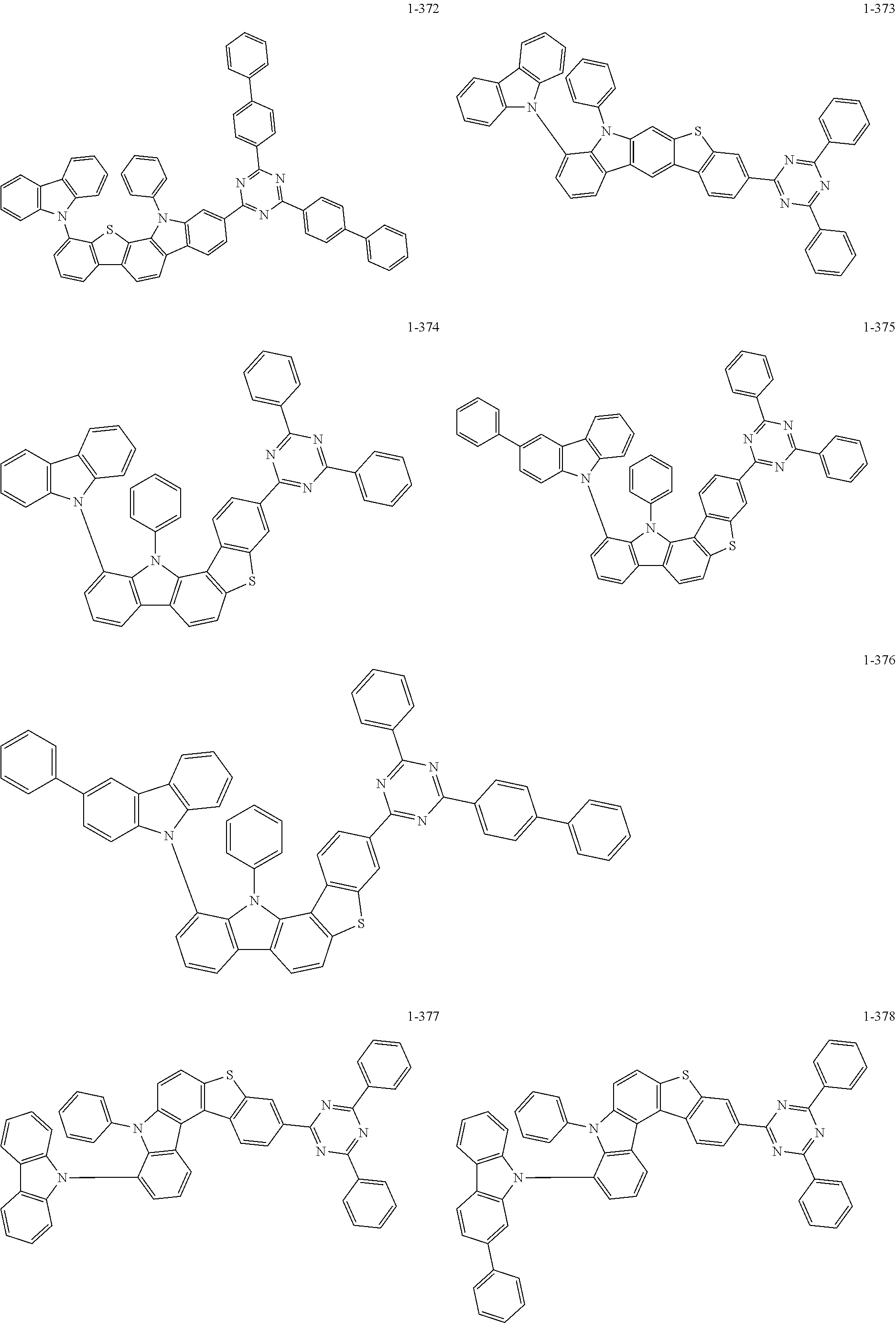

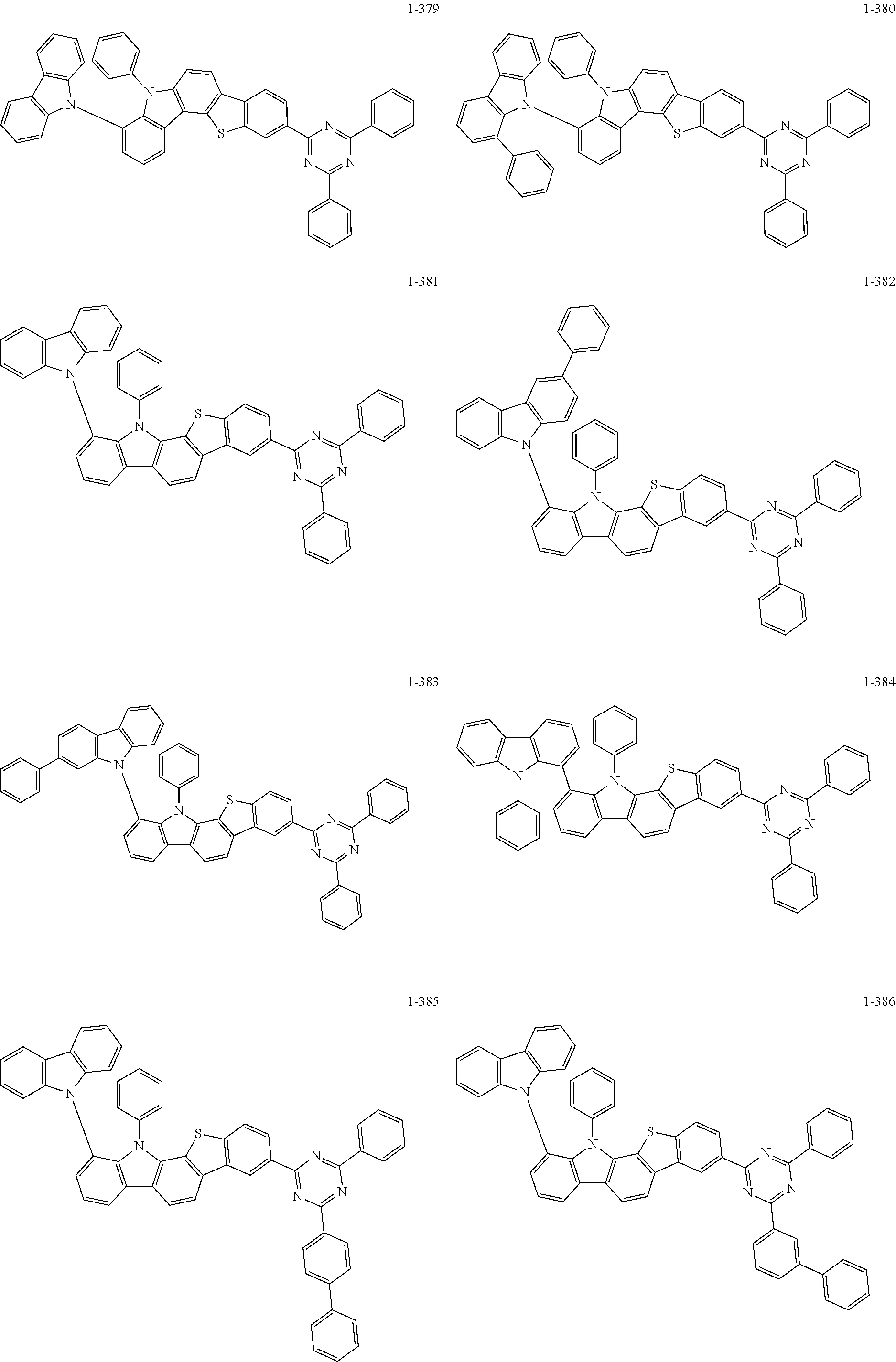

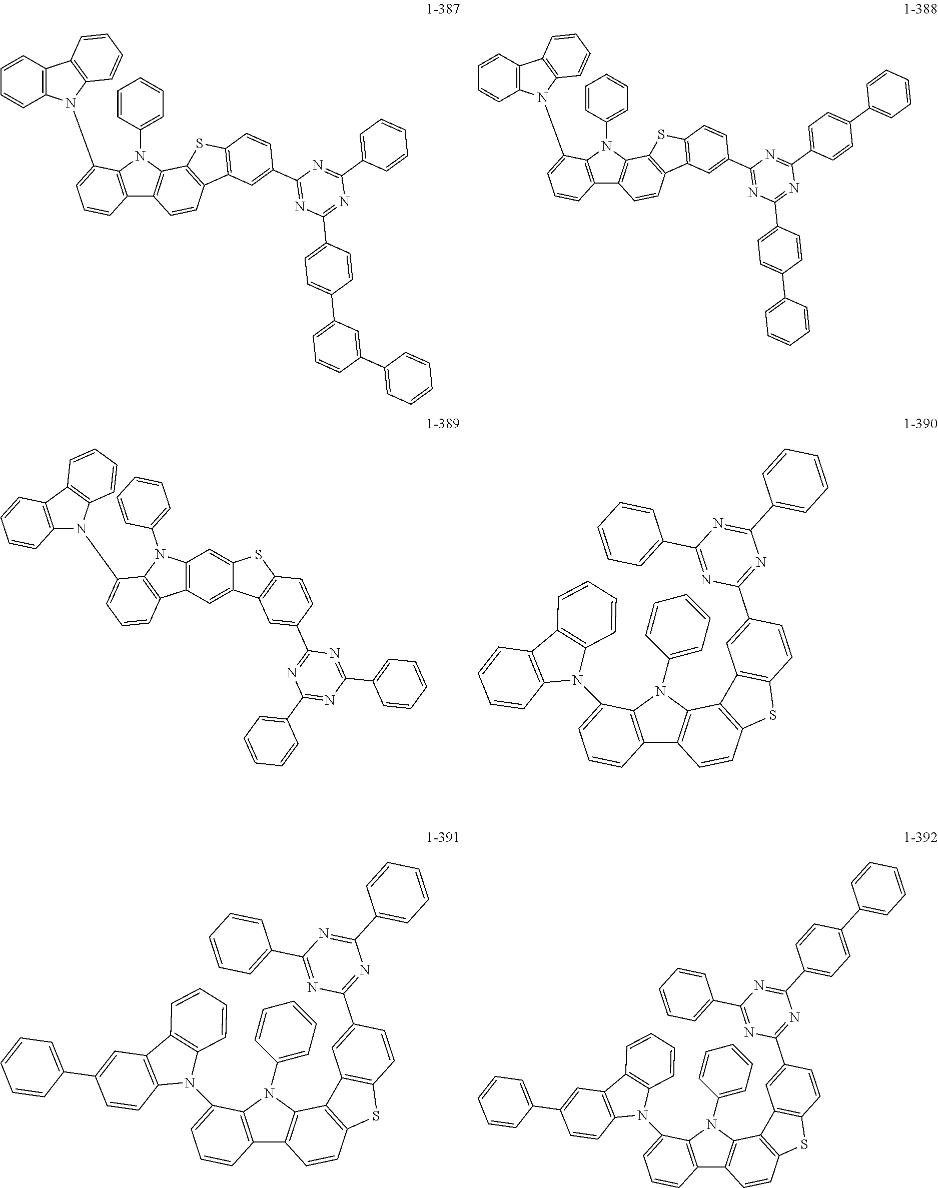

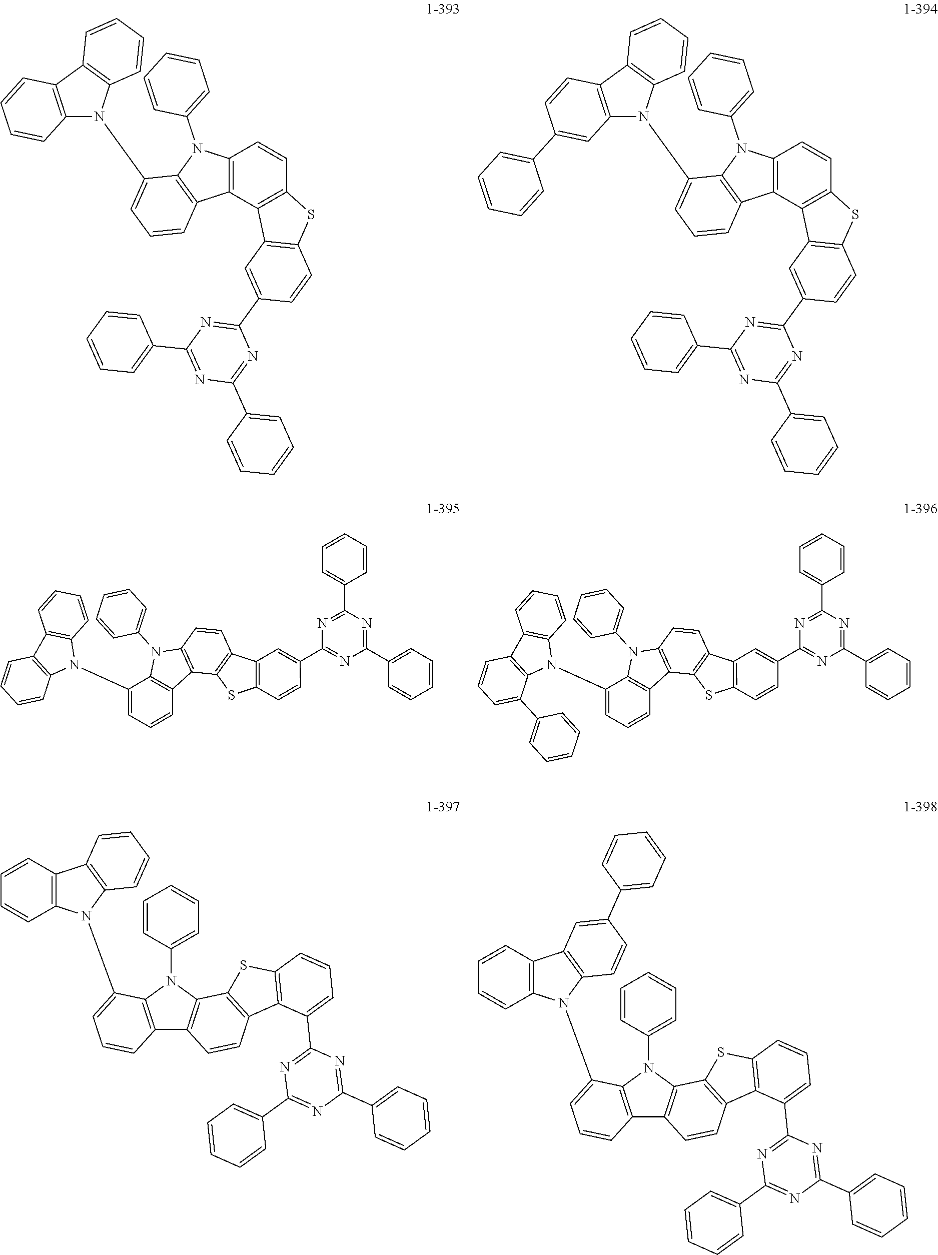

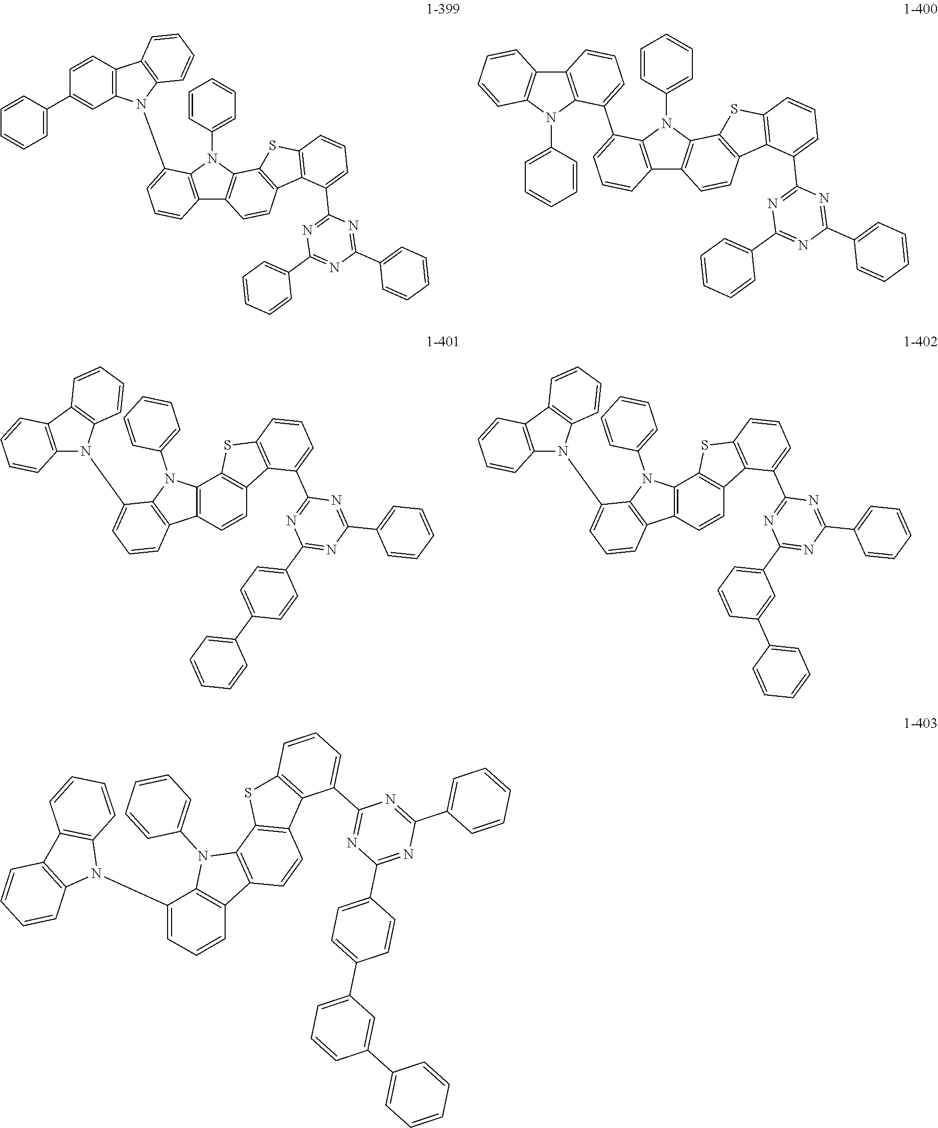

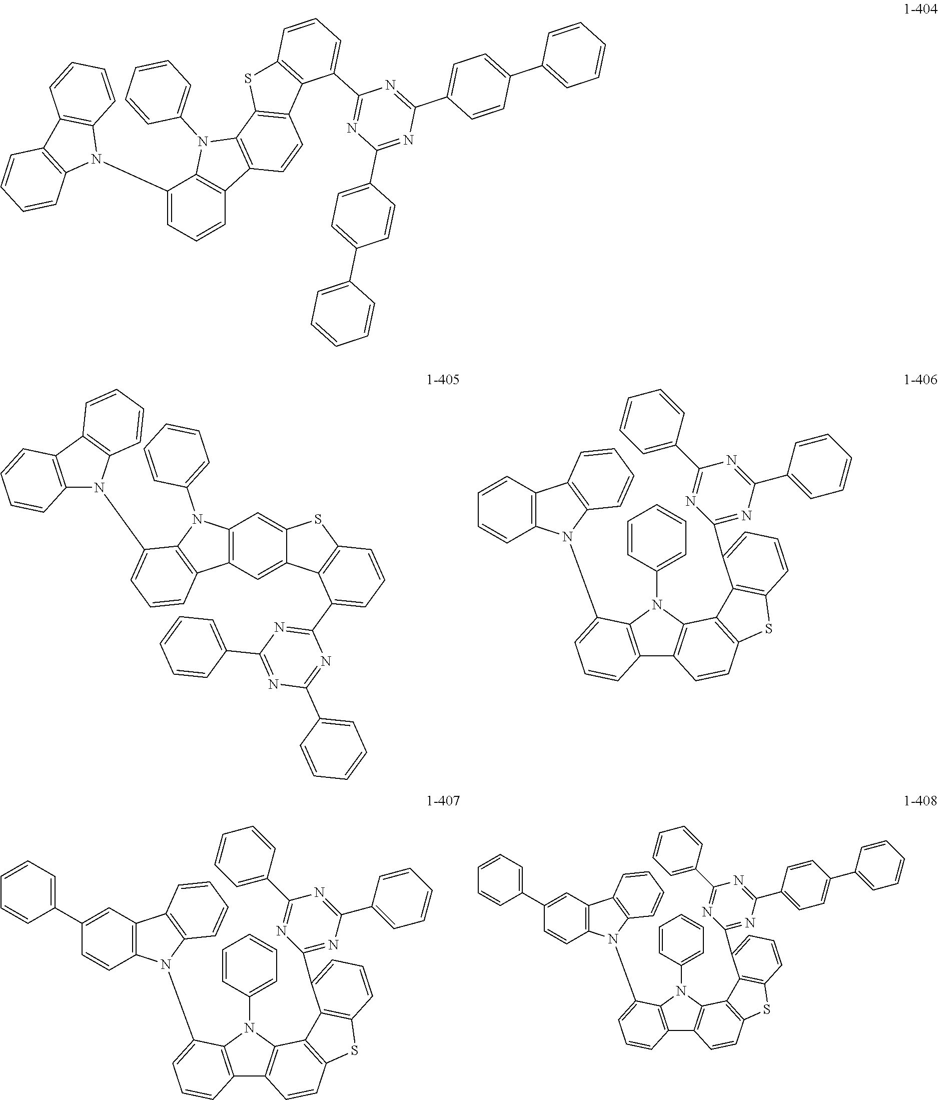

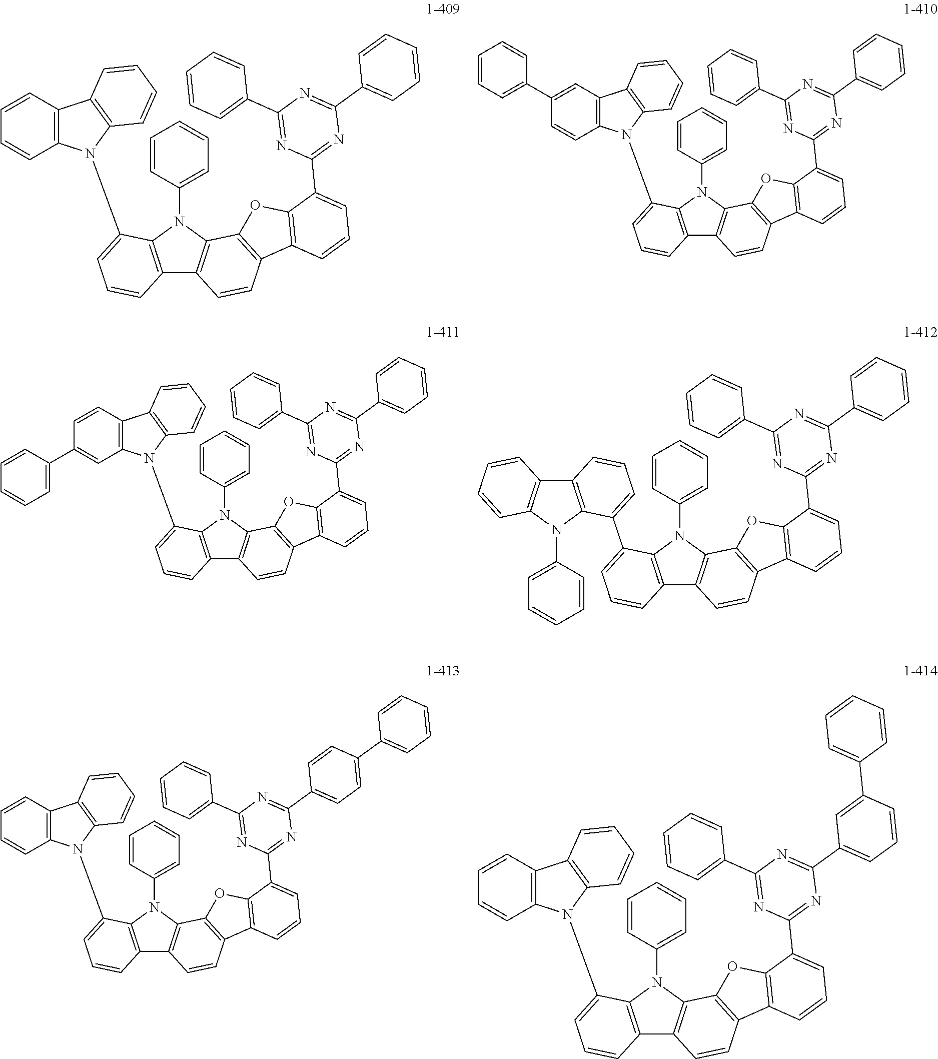

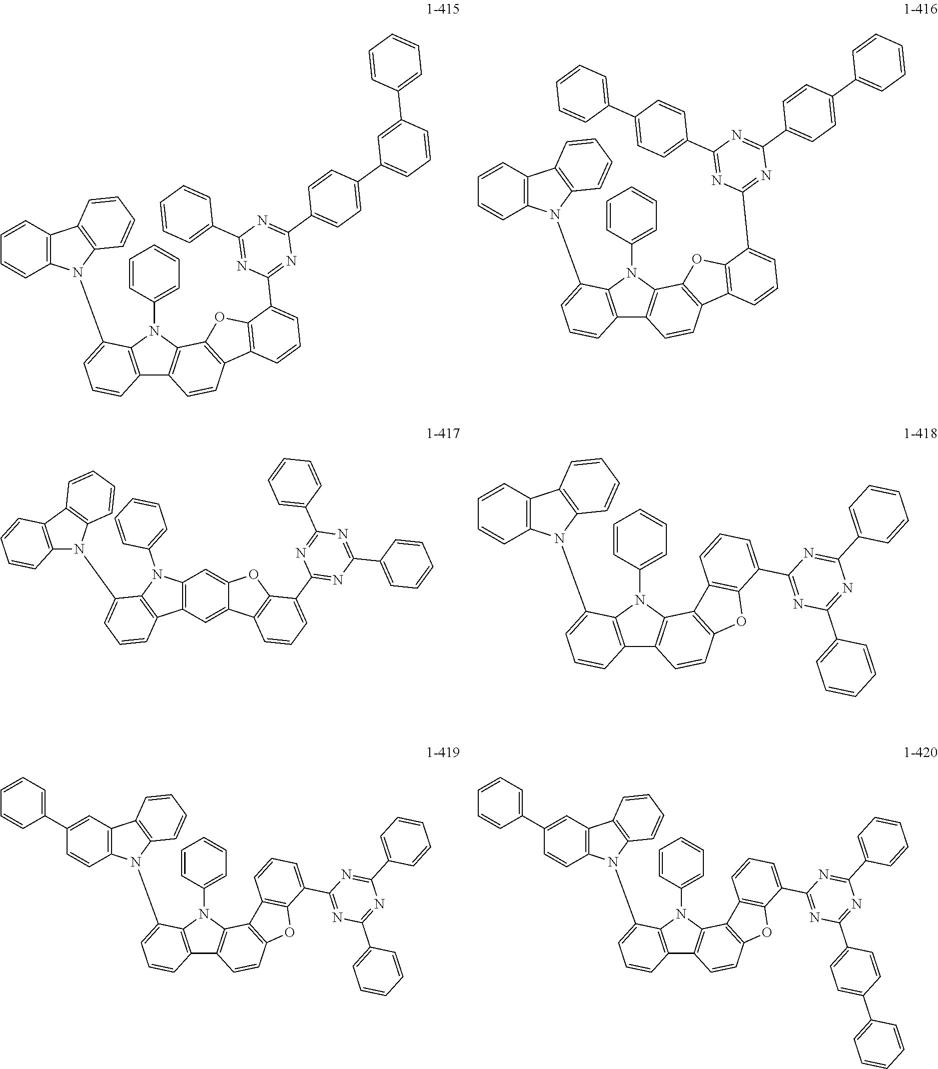

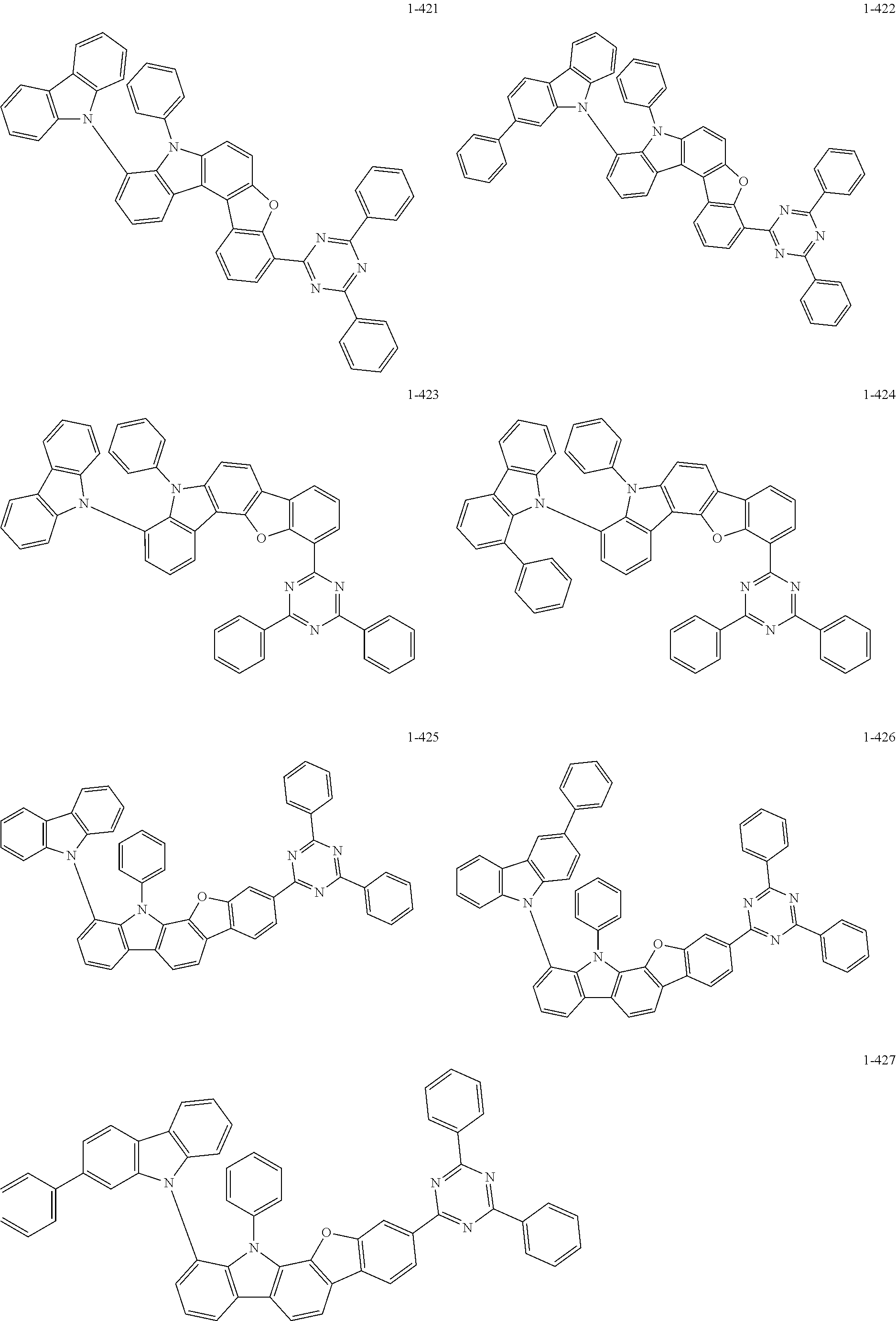

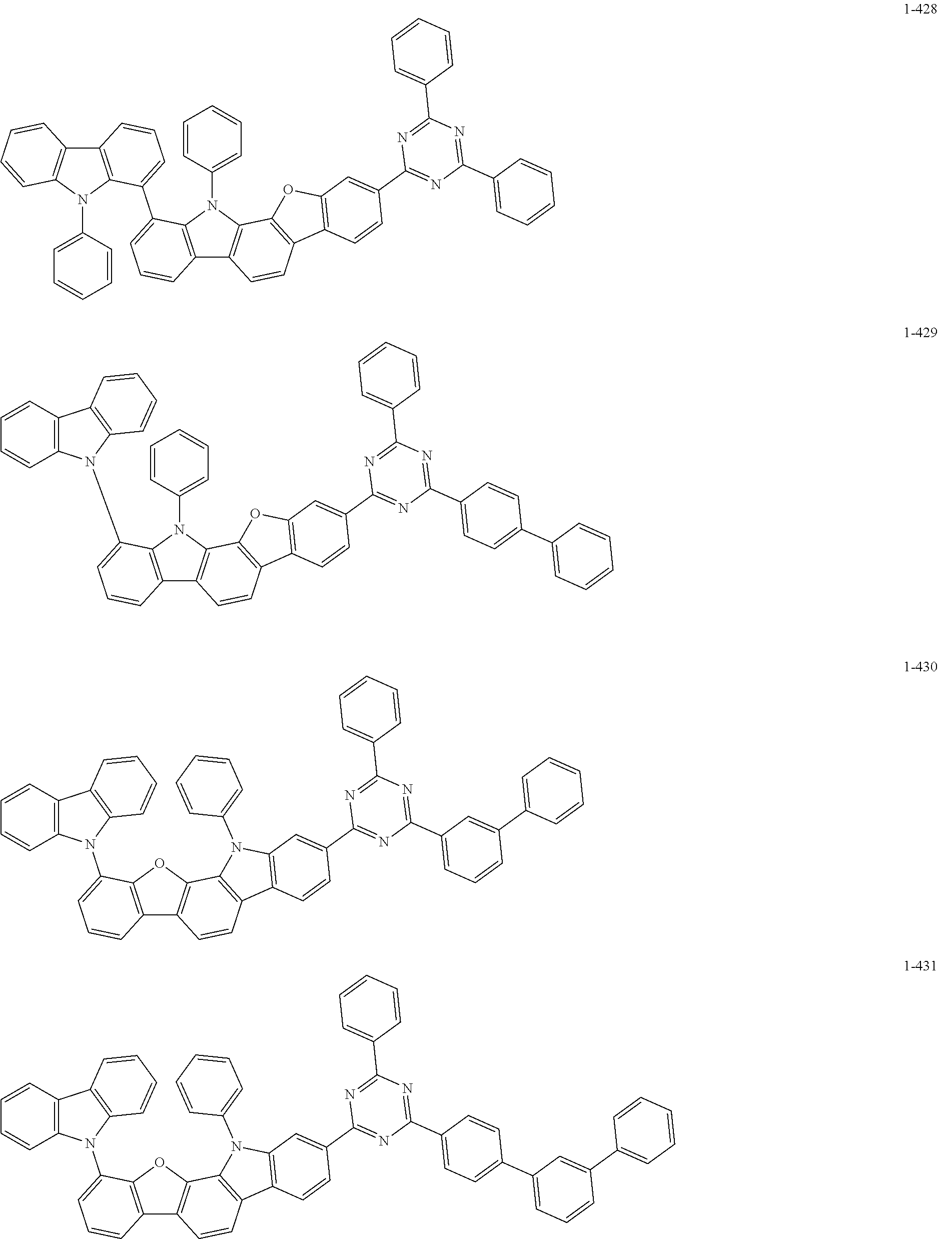 [SMILES: c1ccc(-c2nc(-c3ccccc3)nc(-c3ccc4c(c3)oc3c4ccc4c5cccc(-c6cccc7c8ccccc8n(-c8ccccc8)c67)c5n(-c5ccccc5)c43)n2)cc1 ; c1ccc(-c2ccc(-c3nc(-c4ccccc4)nc(-c4ccc5c(c4)oc4c5ccc5c6cccc(-n7c8ccccc8c8ccccc87)c6n(-c6ccccc6)c54)n3)cc2)cc1 ; c1ccc(-c2cccc(-c3nc(-c4ccccc4)nc(-c4ccc5c6ccc7c8cccc(-n9c%10ccccc%10c%10ccccc%109)c8oc7c6n(-c6ccccc6)c5c4)n3)c2)cc1 ; c1ccc(-c2cccc(-c3ccc(-c4nc(-c5ccccc5)nc(-c5ccc6c7ccc8c9cccc(-n%10c%11ccccc%11c%11ccccc%11%10)c9oc8c7n(-c7ccccc7)c6c5)n4)cc3)c2)cc1]

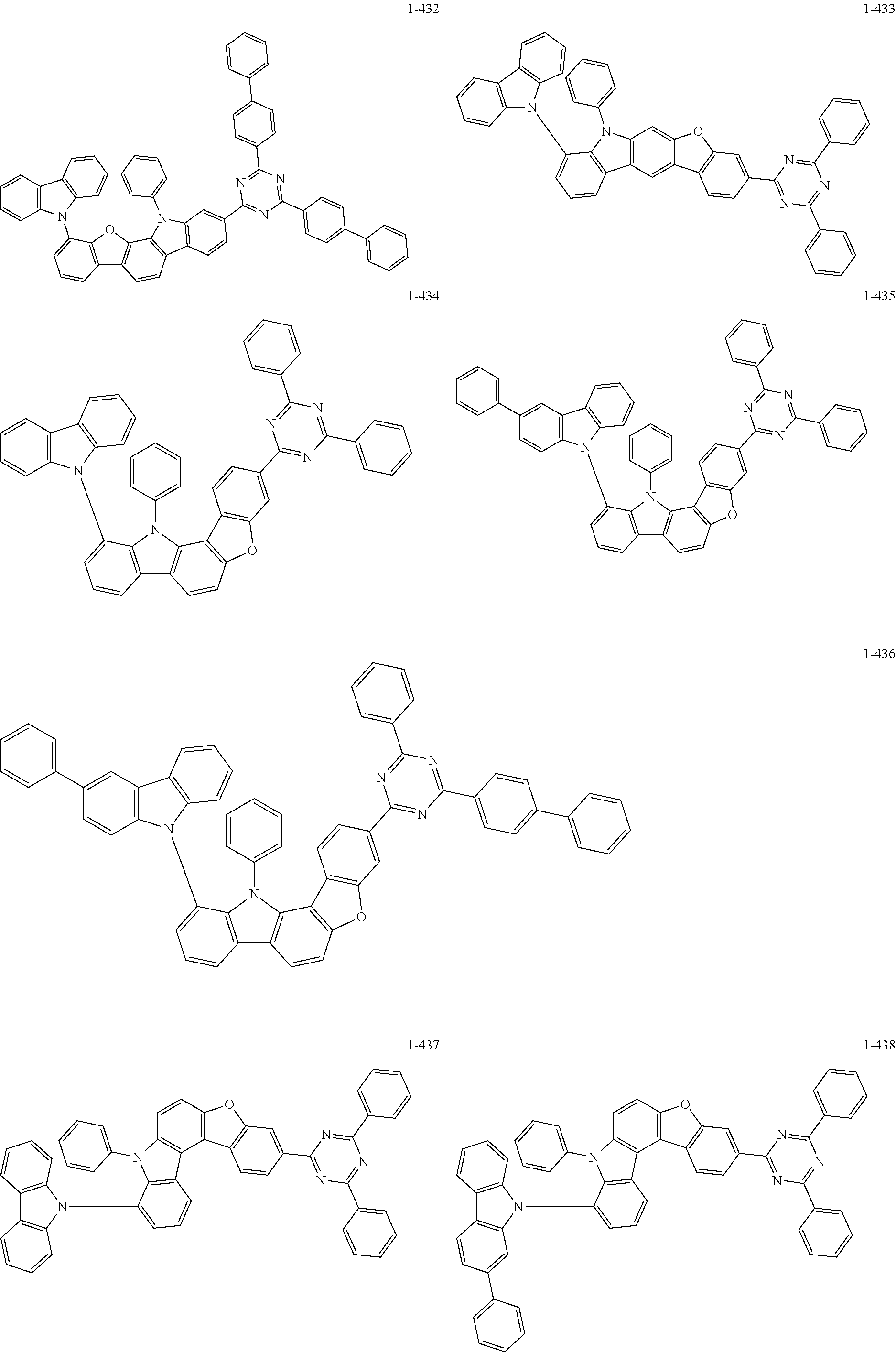

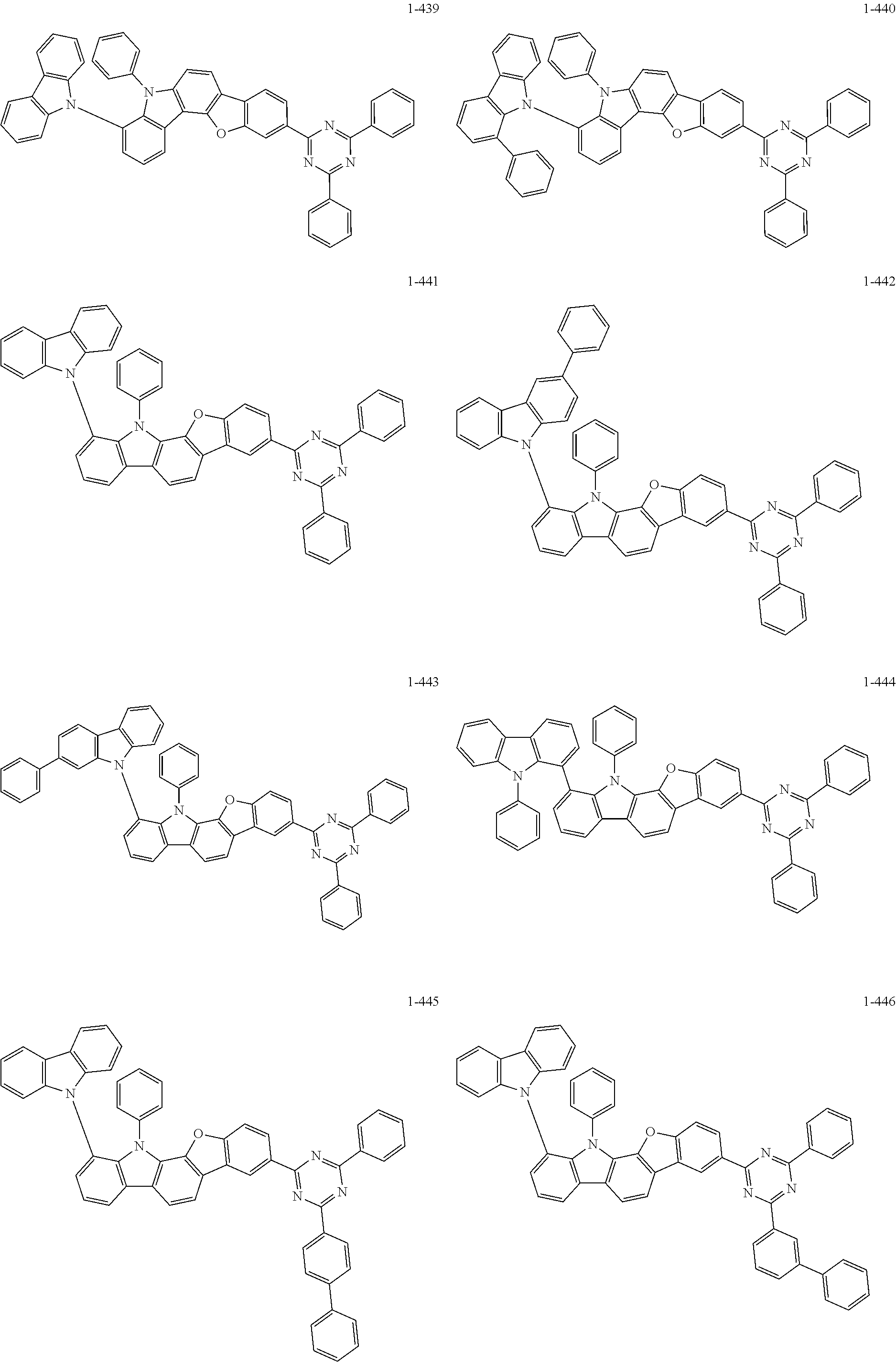

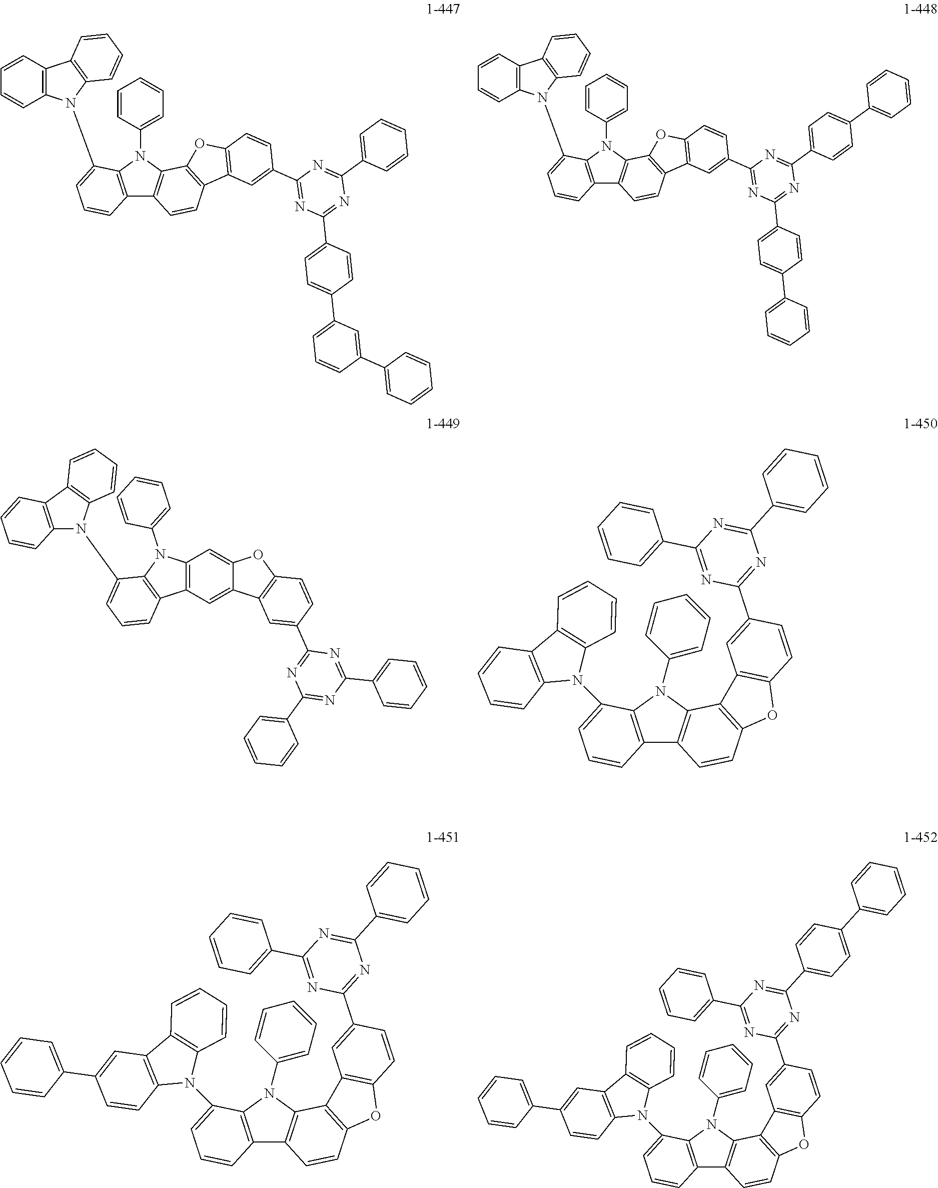

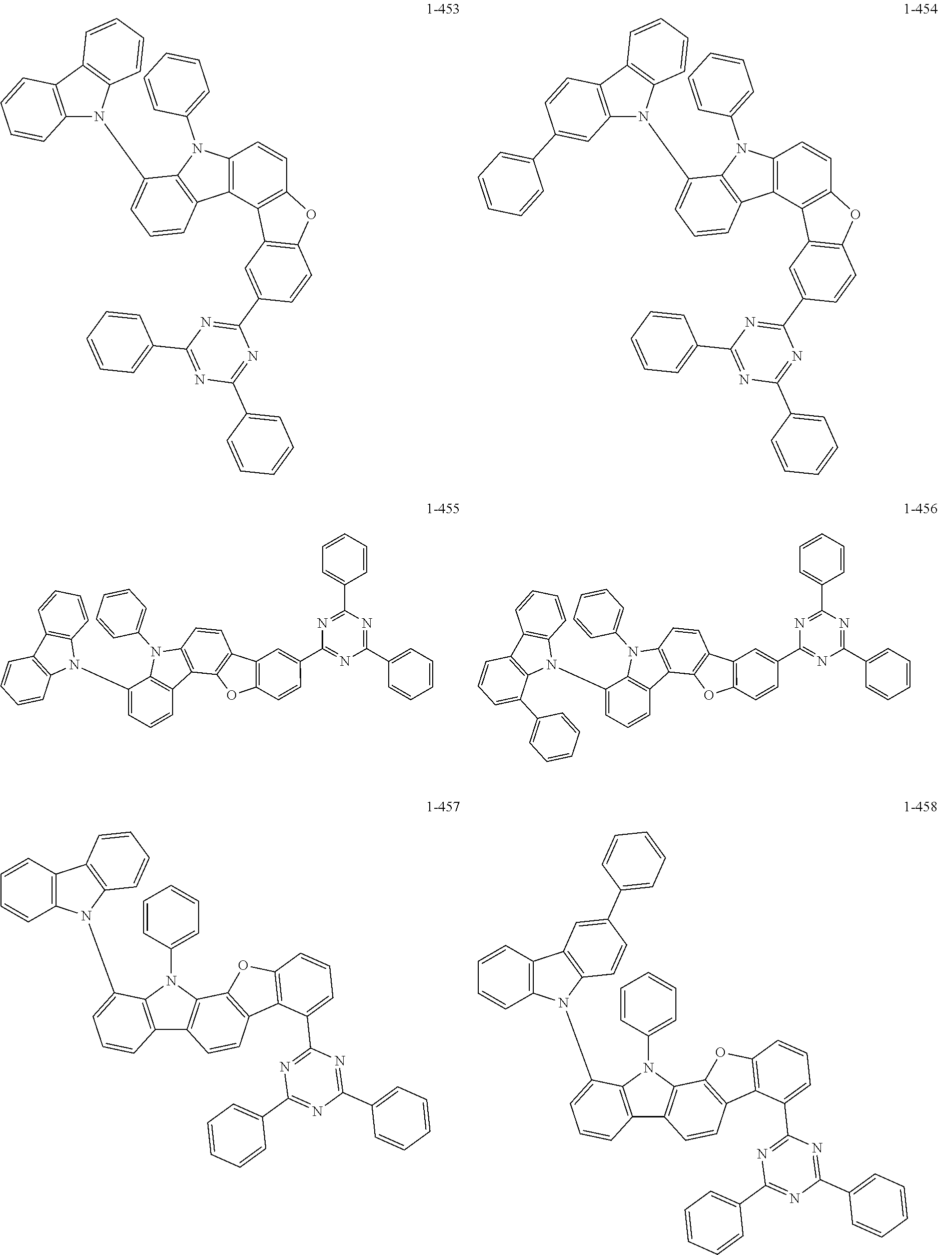

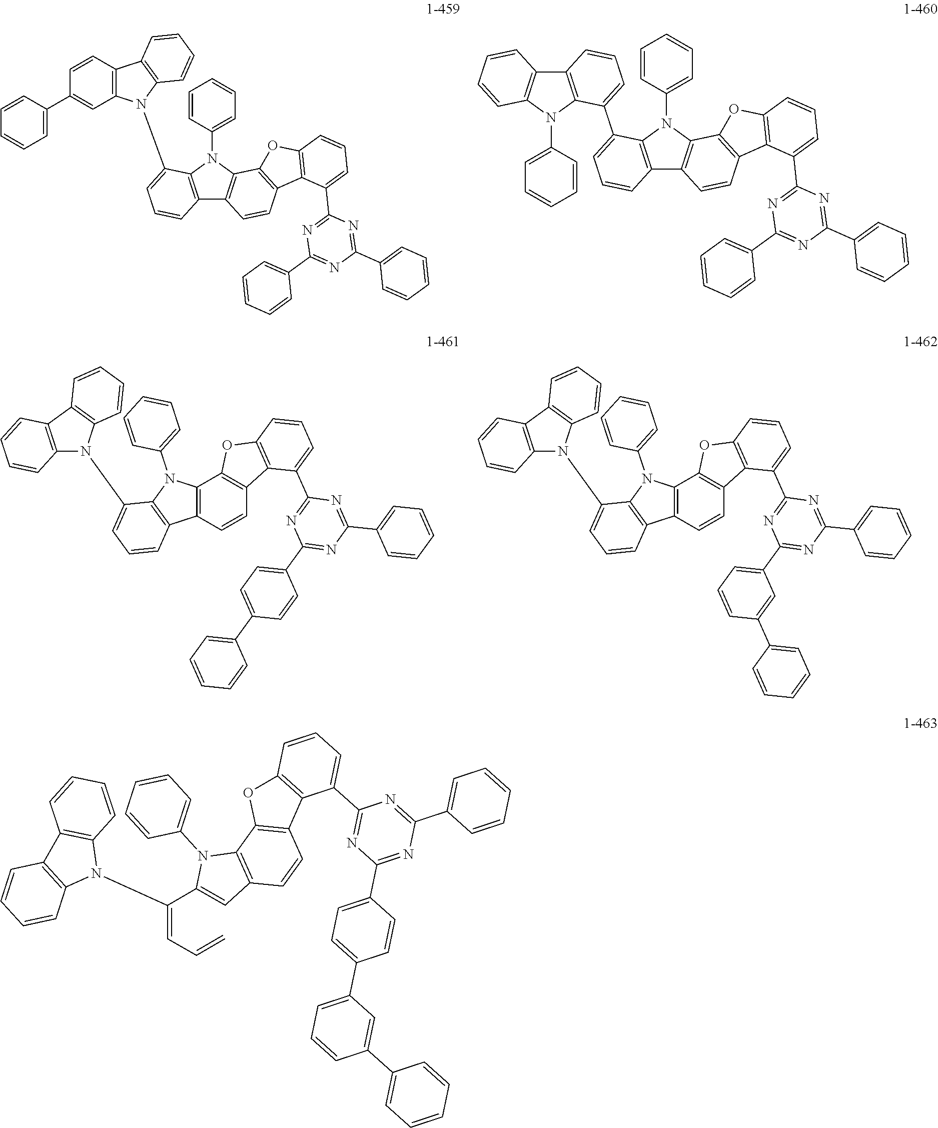

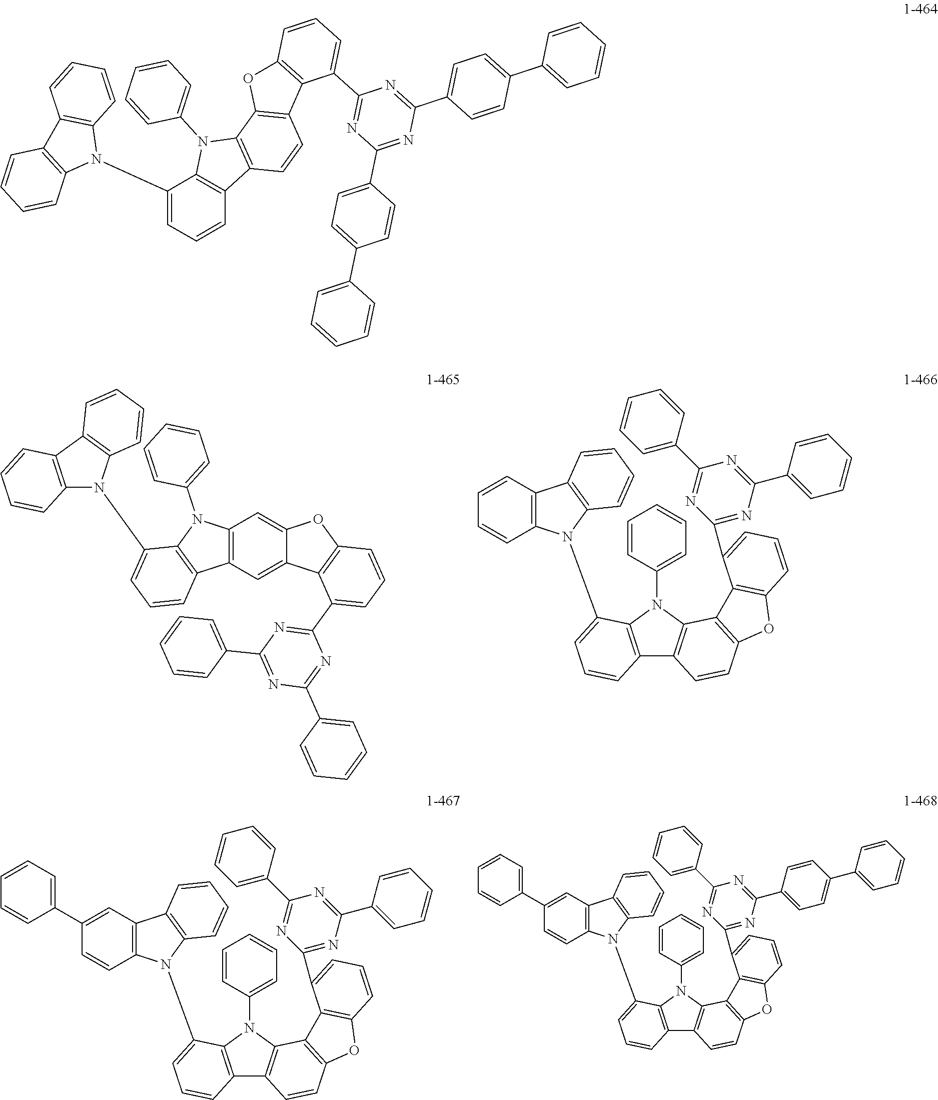

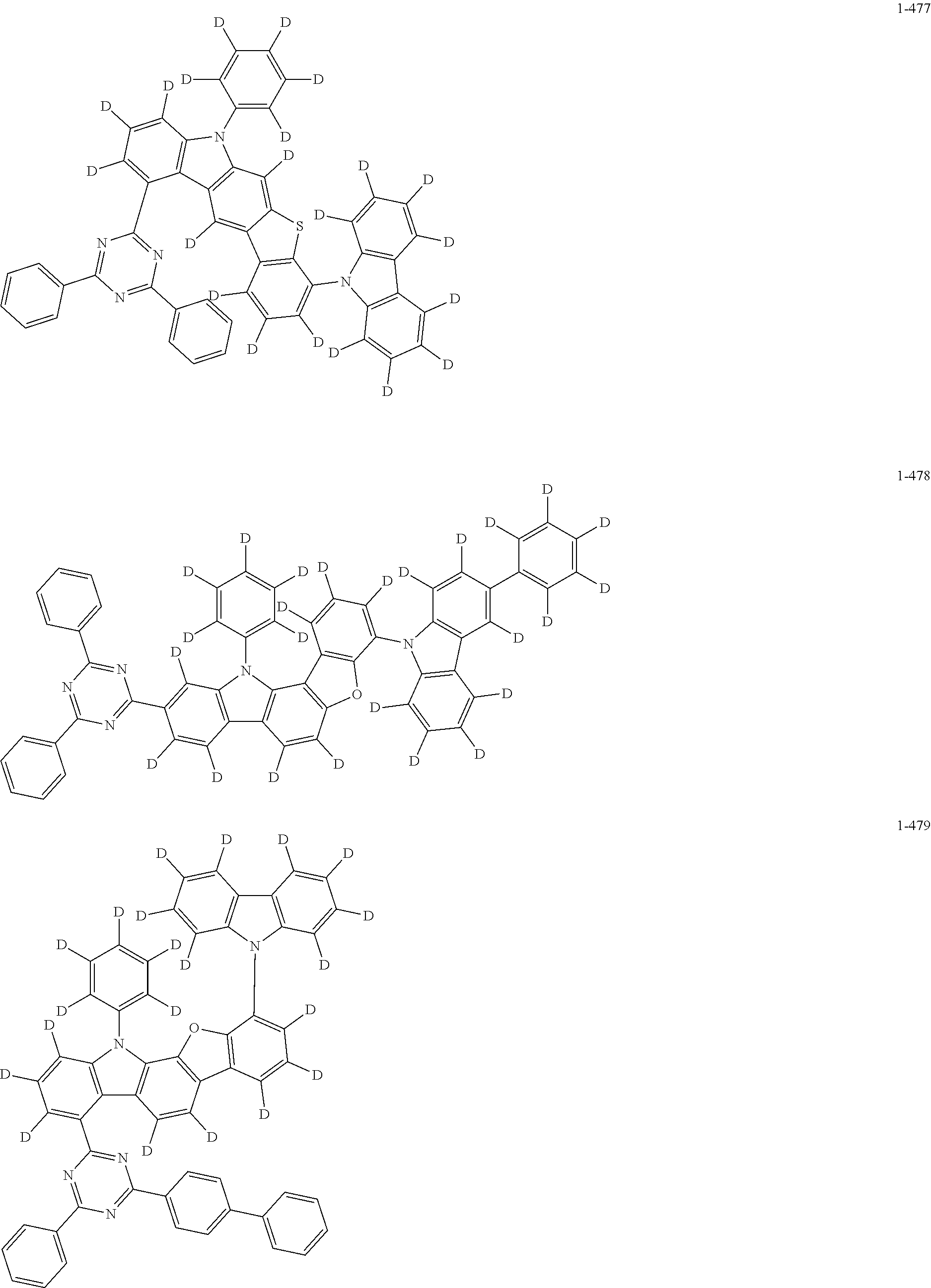

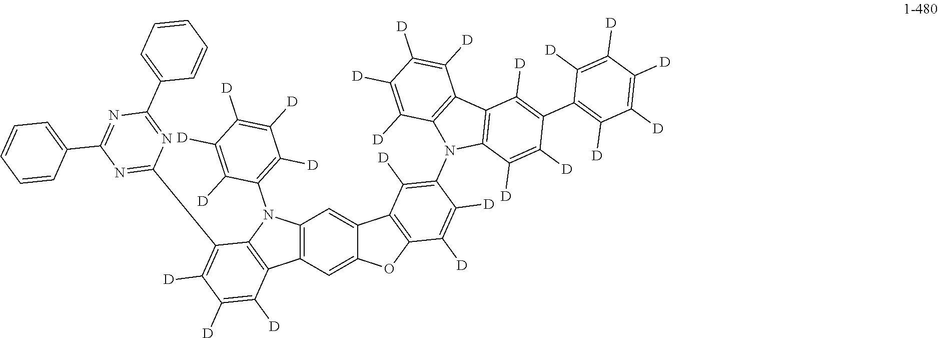 [SMILES: [2H]c1c([2H])c([2H])c(-c2c([2H])c([2H])c3c(c2[2H])c2c([2H])c([2H])c([2H])c([2H])c2n3-c2c([2H])c([2H])c3oc4cc5c6c([2H])c([2H])c([2H])c(-c7nc(-c8ccccc8)nc(-c8ccccc8)n7)c6n(-c6c([2H])c([2H])c([2H])c([2H])c6[2H])c5cc4c3c2[2H])c([2H])c1[2H]]

7. An organic light emitting device comprising: a first electrode; a second electrode provided to face the first electrode; and an organic material layer having one or more layers provided between the first electrode and the second electrode, wherein one or more layers of the organic material layer comprise the heterocylic compound according to claim 1.

8. The organic light emitting device of claim 7, wherein the organic material layer comprising the heterocyclic compound further comprises a heterocyclic compound represented by the following Chemical Formula 2: 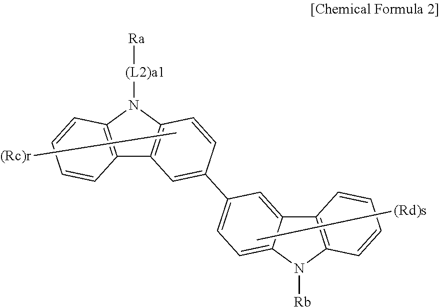
in Chemical Formula 2,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′, or two or more adjacent groups are bonded to each other to form a substituted or unsubstituted aromatic hydrocarbon ring or a substituted or unsubstituted hetero ring,
L2 is a direct bond; a substituted or unsubstituted C6 to C60 arylene group; or a substituted or unsubstituted C2 to C60 heteroarylene group,
Ra and Rb are the same as or different from each other, and are each independently —CN; —SiRR′R″; —P(═O)RR′; a substituted or unsubstituted C6 to C60 aryl group; or a substituted or unsubstituted C2 to C60 heteroaryl group,
R, R′ and R″ are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
a1 is an integer from 0 to 4,
r and s are an integer from 0 to 7, and
when a1, s and r are 2 or higher, substituents in the parenthesis are the same as or different from each other.

9. The organic light emitting device of claim 8, wherein the heterocyclic compound represented by Chemical Formula 2 is any one selected from the following compounds:

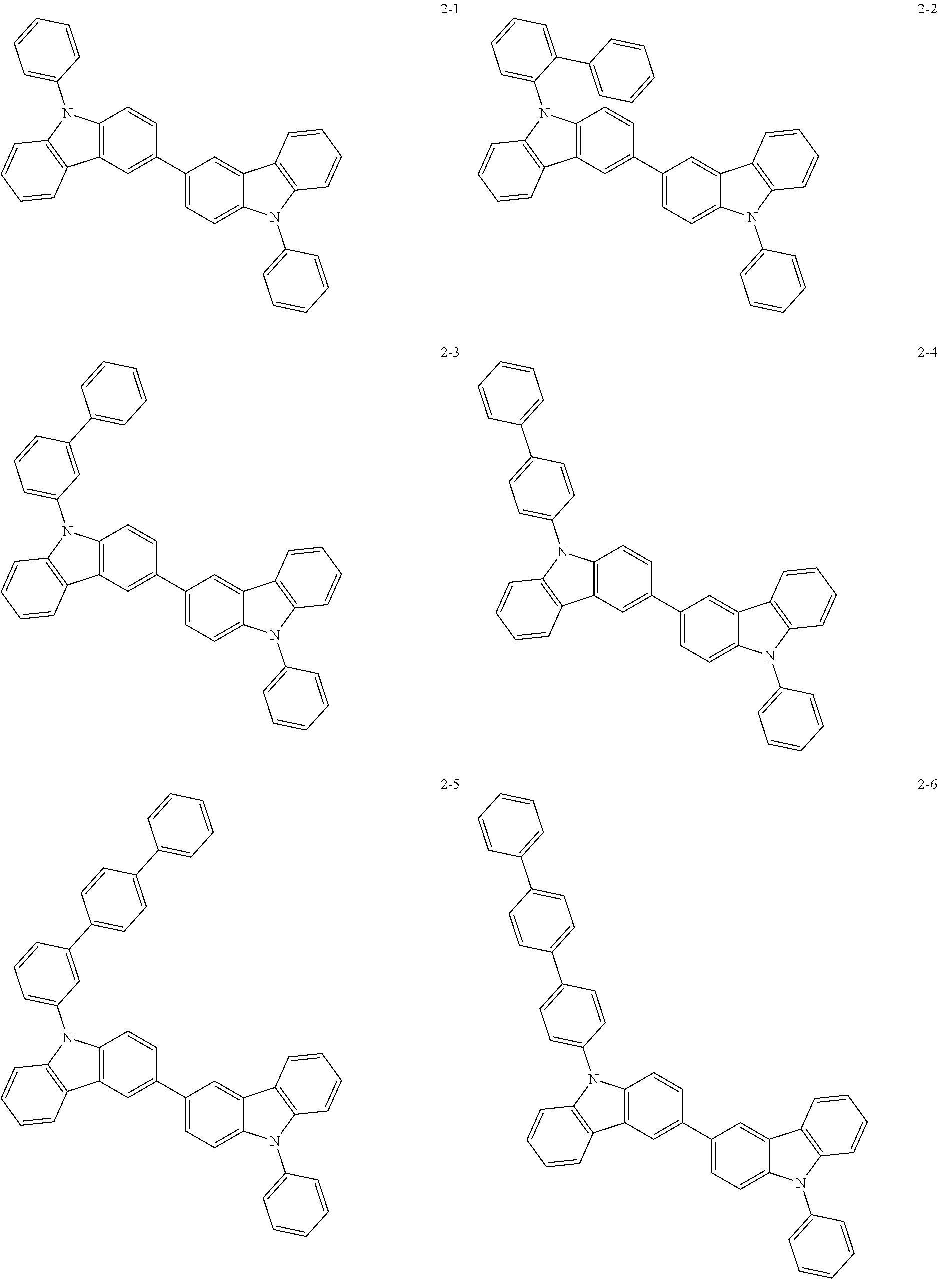 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

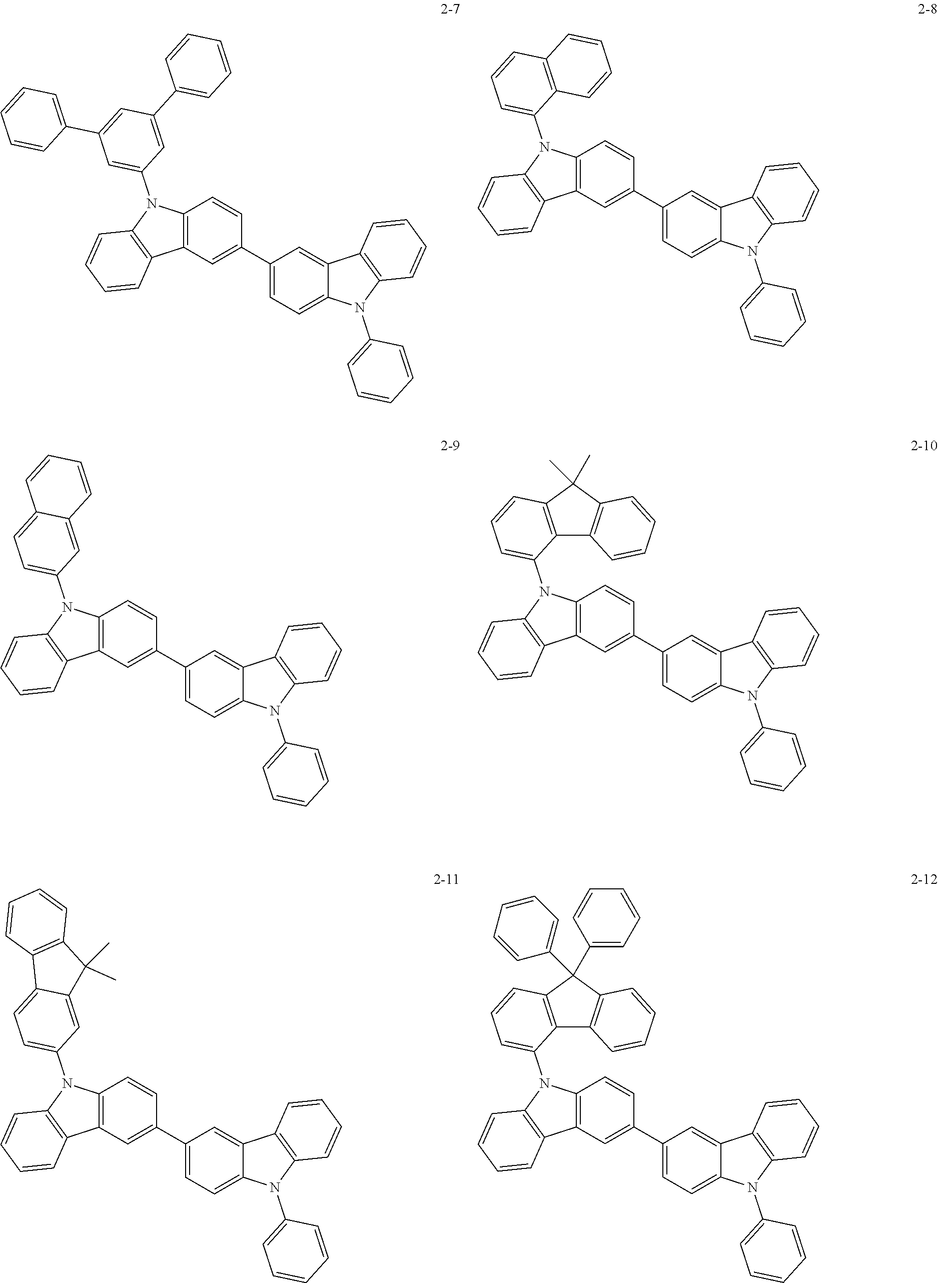 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

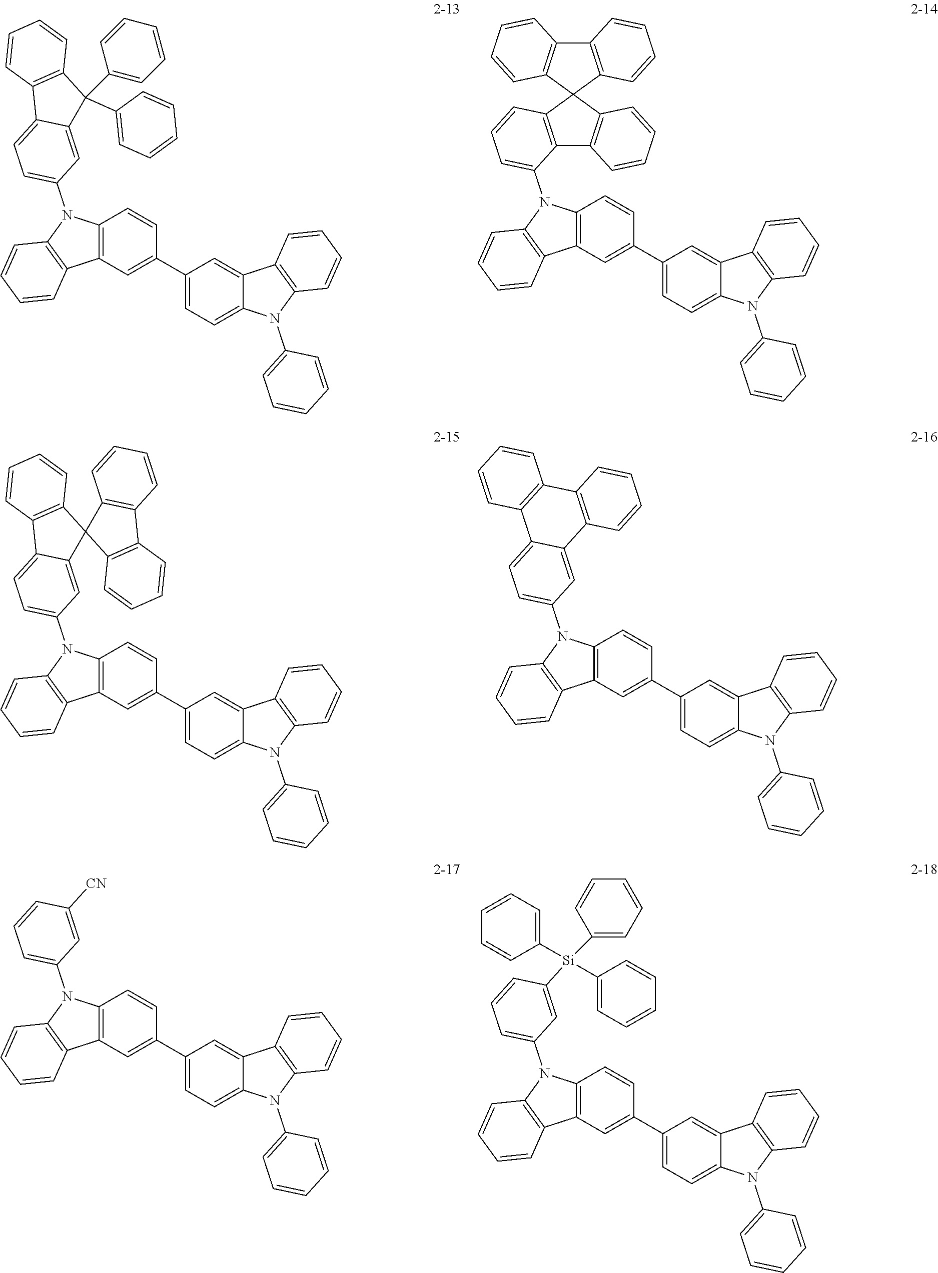

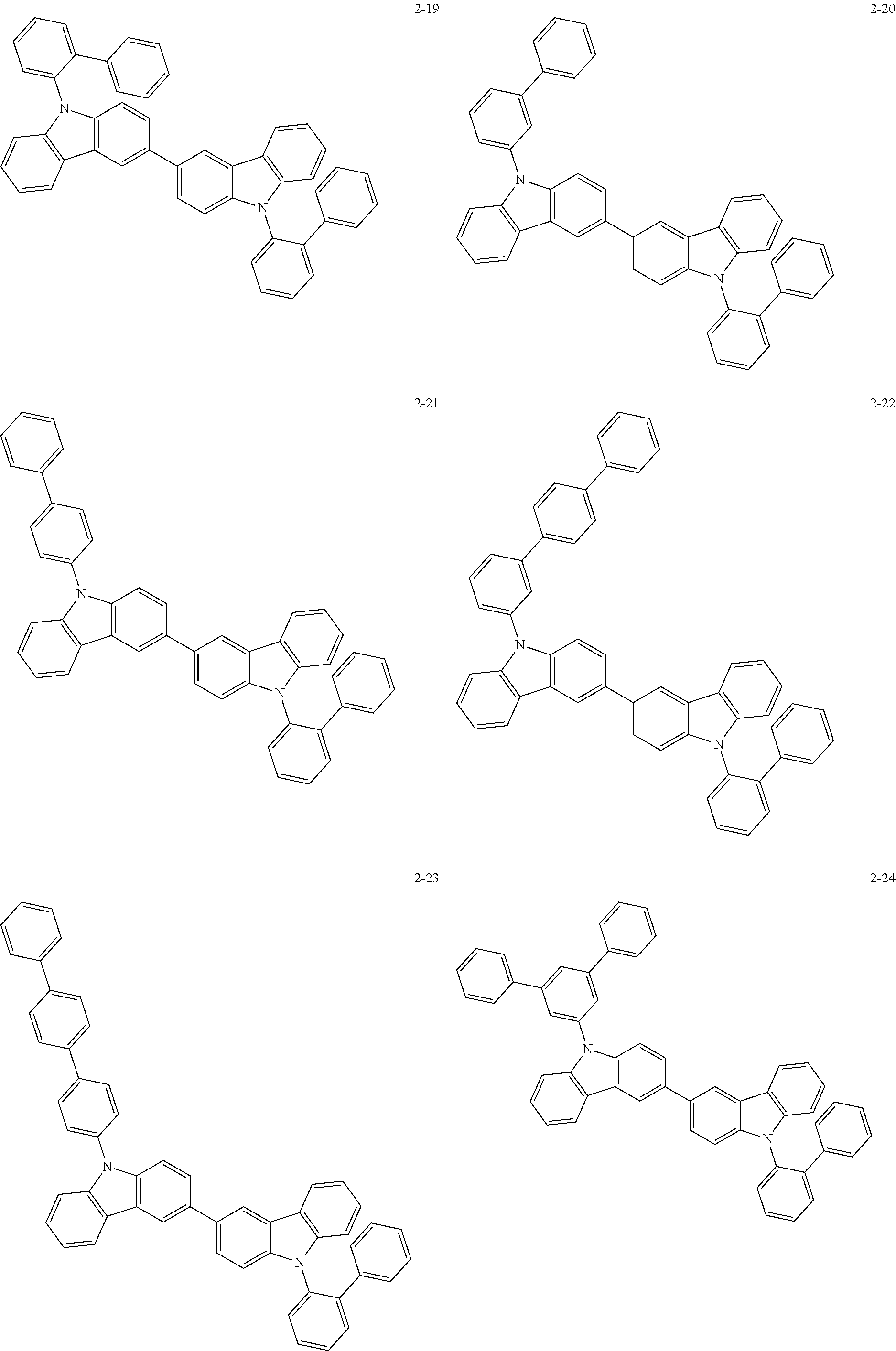

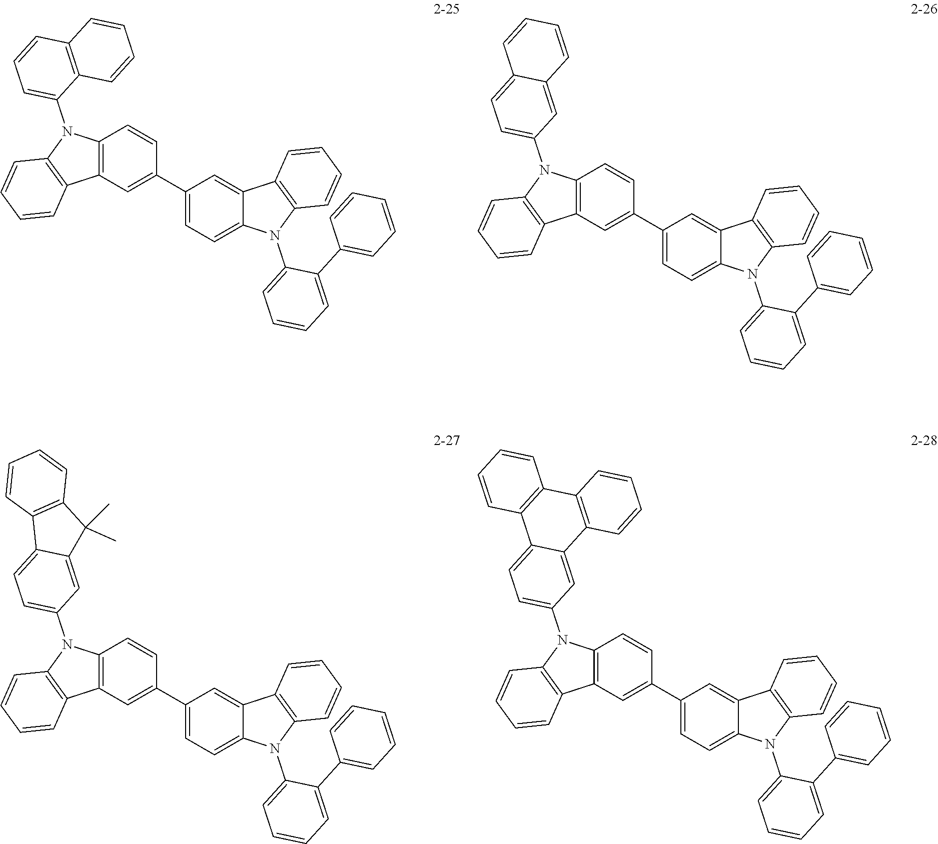 [SMILES: c1ccc(-c2ccccc2-n2c3ccccc3c3cc(-c4ccc5c(c4)c4ccccc4n5-c4cccc5ccccc45)ccc32)cc1 ; c1ccc(-c2ccccc2-n2c3ccccc3c3cc(-c4ccc5c(c4)c4ccccc4n5-c4ccc5ccccc5c4)ccc32)cc1 ; CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccccc2-n2c3ccccc3c3cc(-c4ccc5c(c4)c4ccccc4n5-c4ccc5c6ccccc6c6ccccc6c5c4)ccc32)cc1]

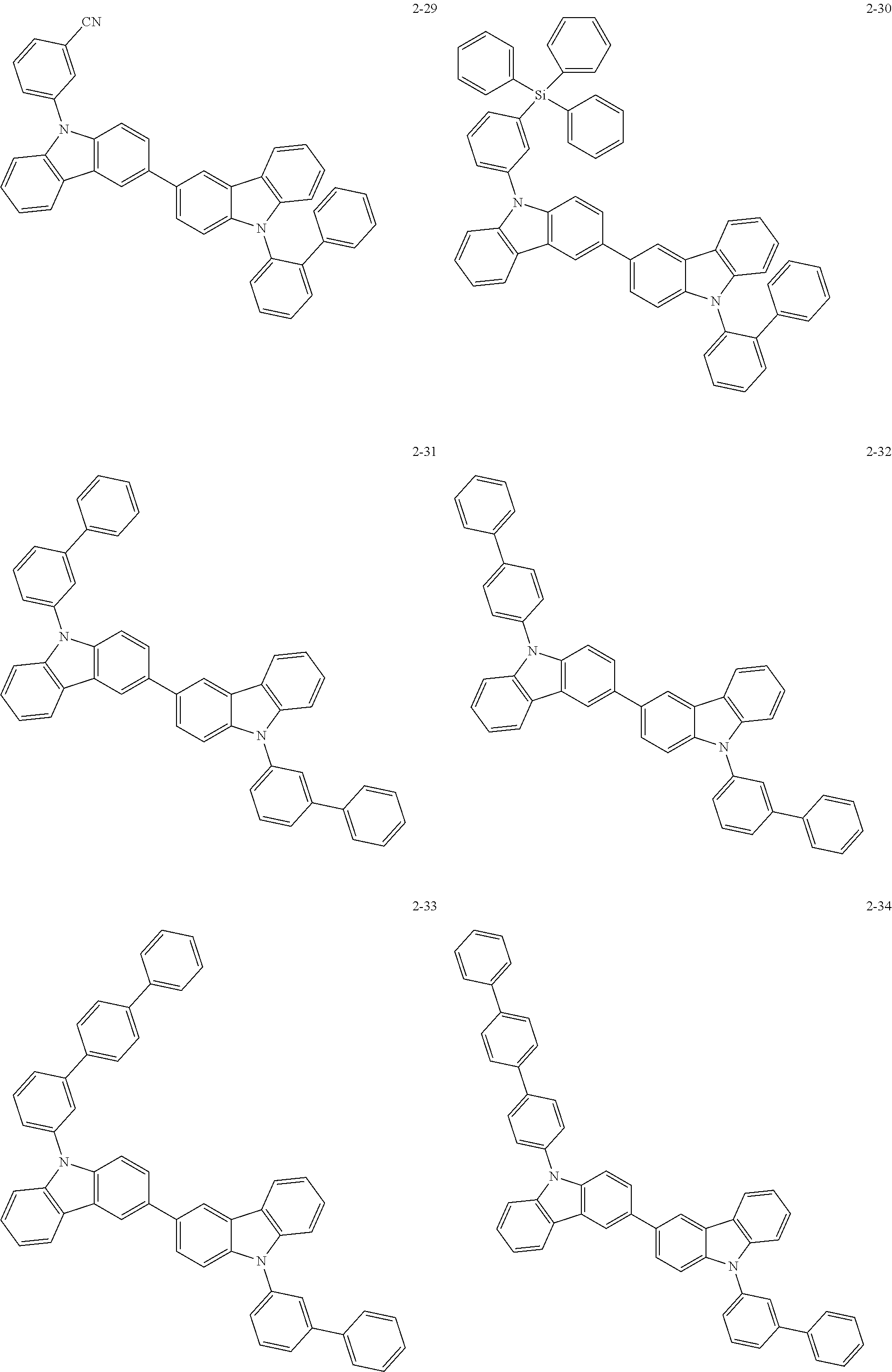

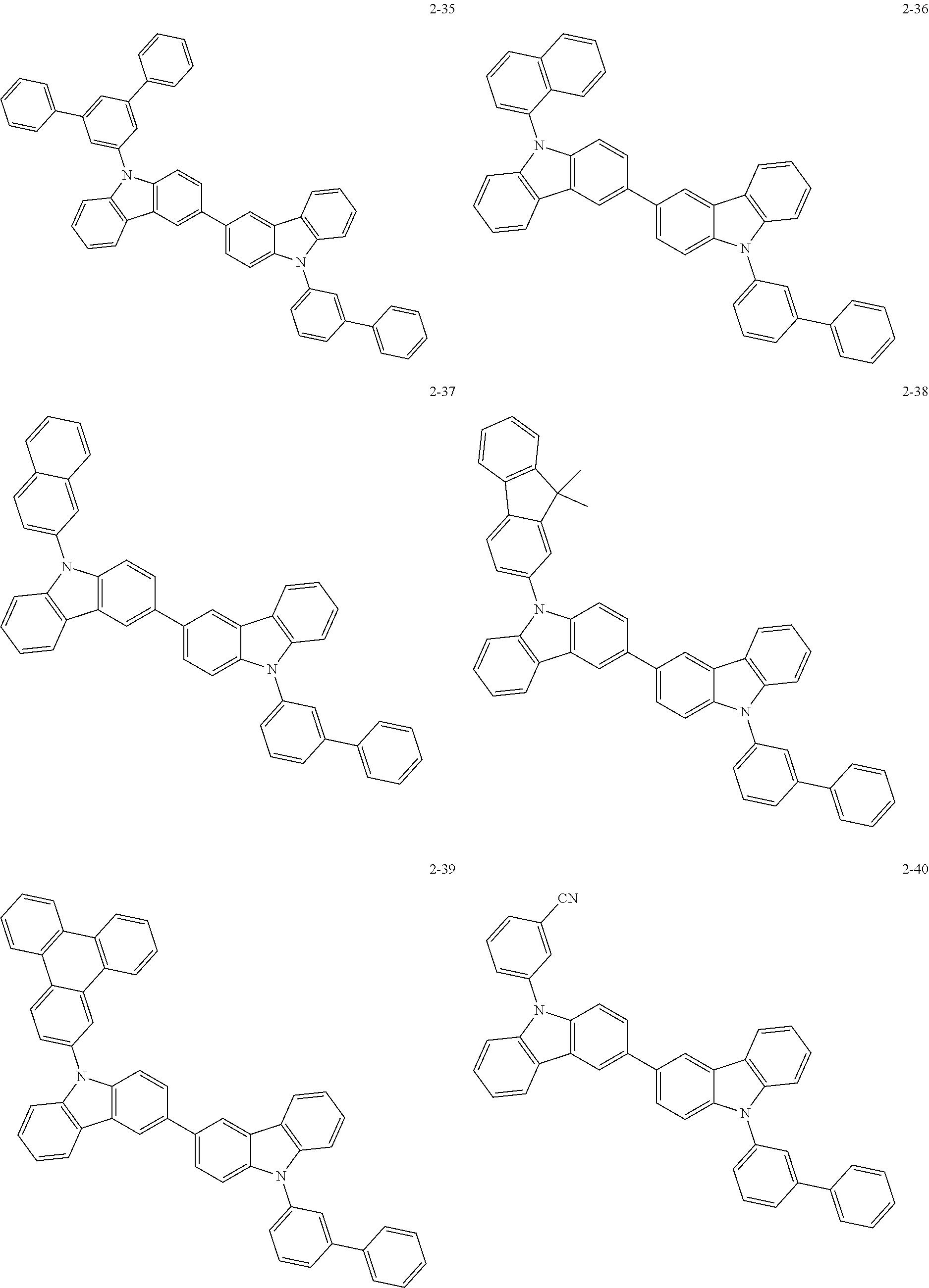

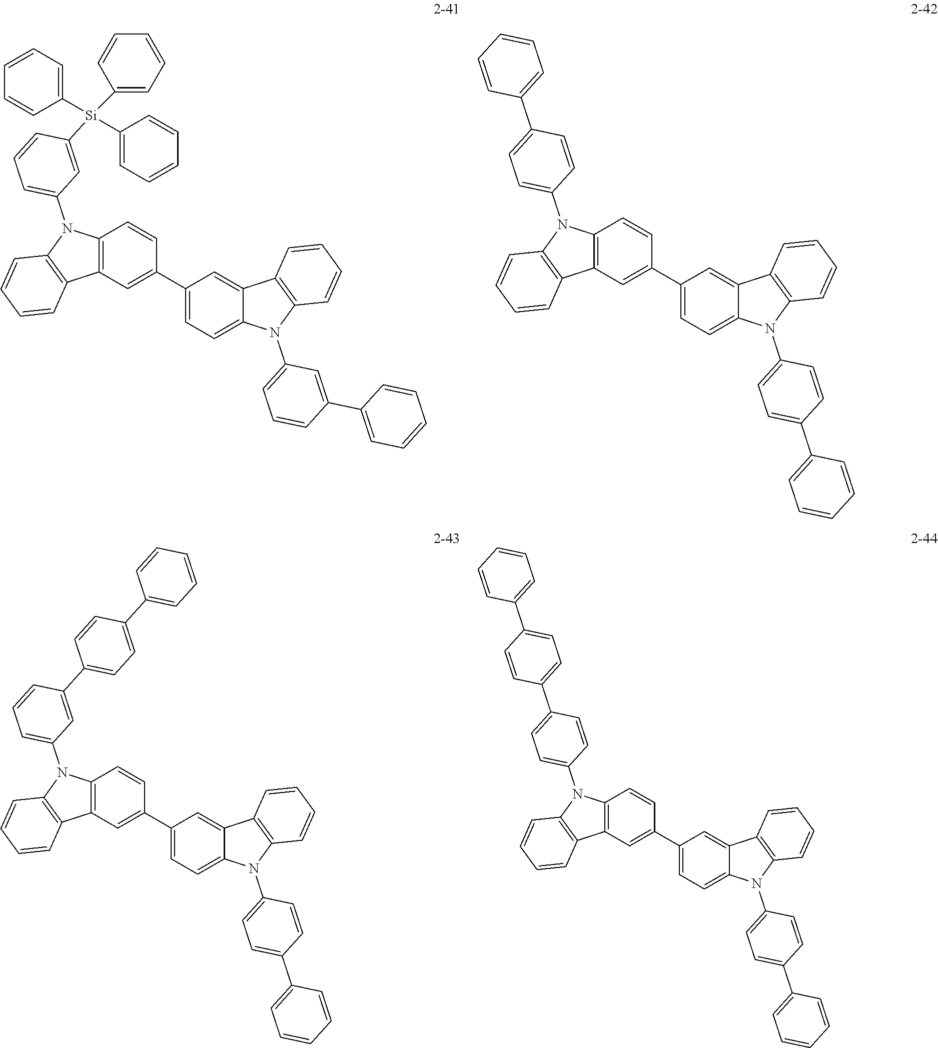 [SMILES: c1ccc(-c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

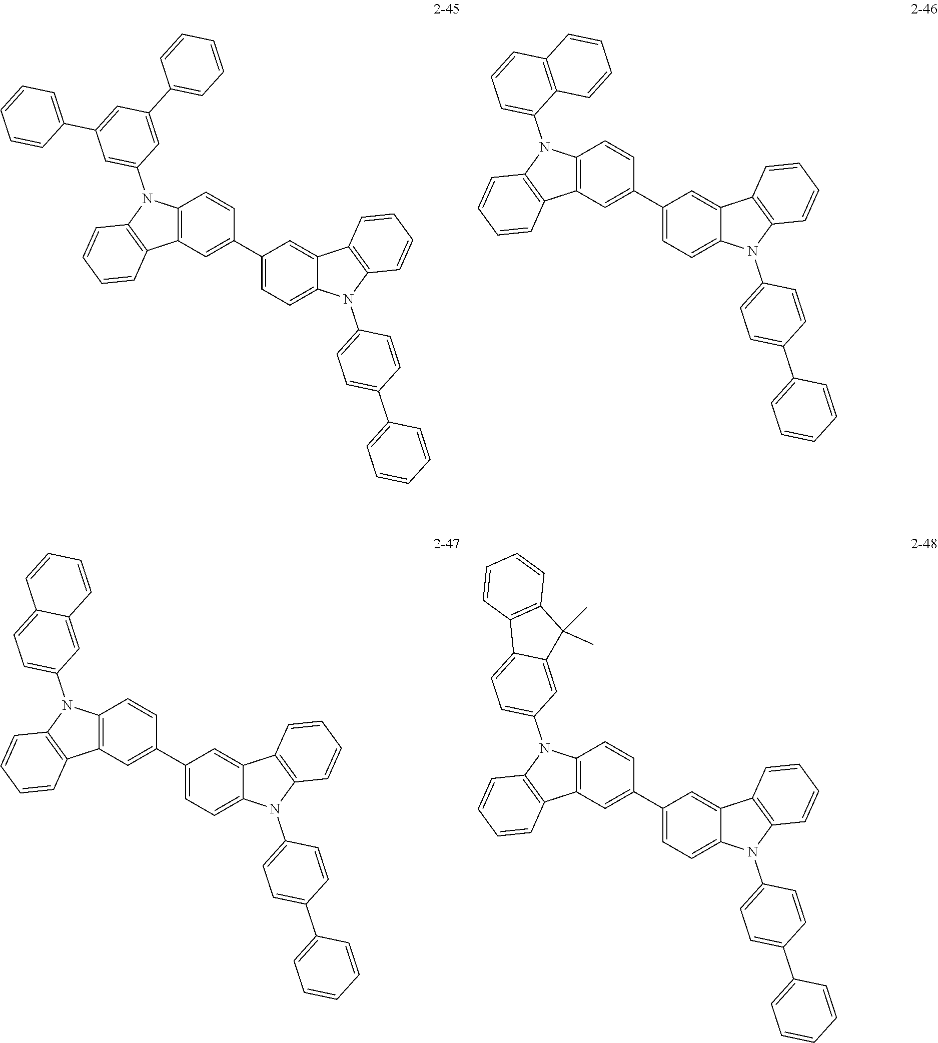 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

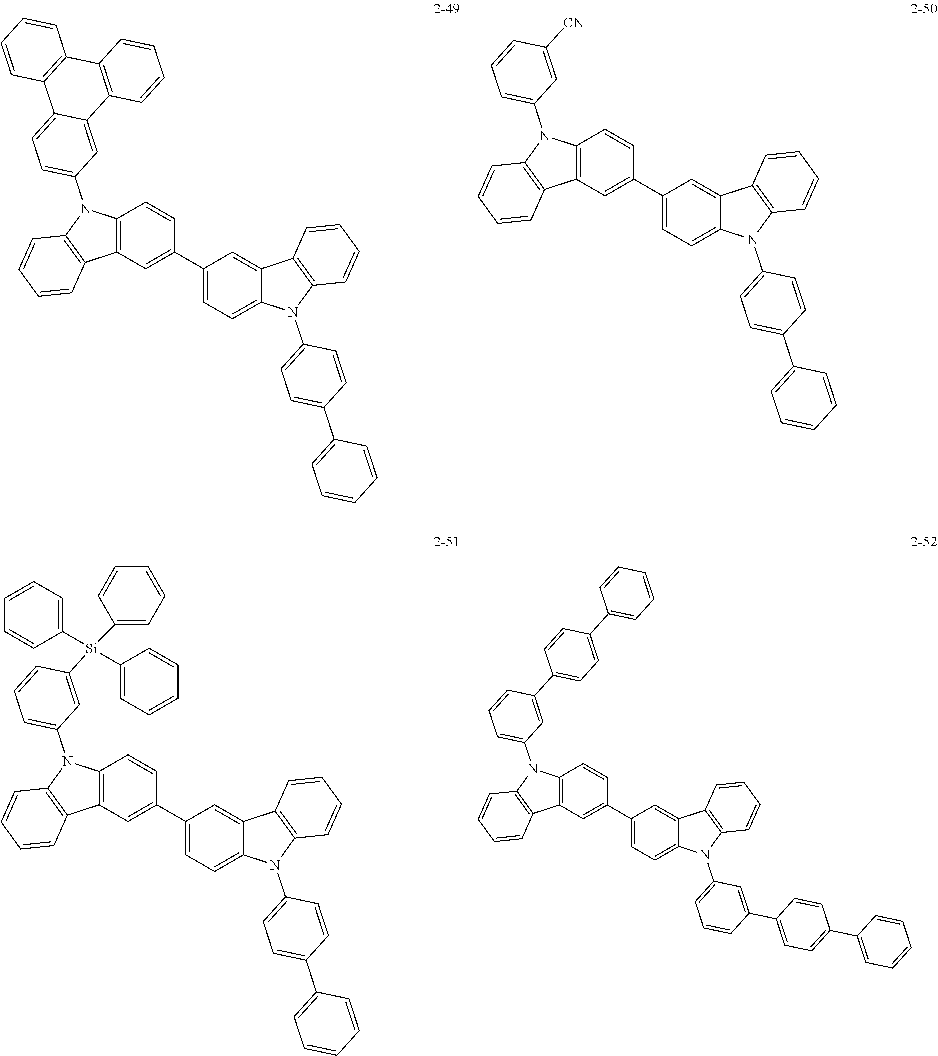 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

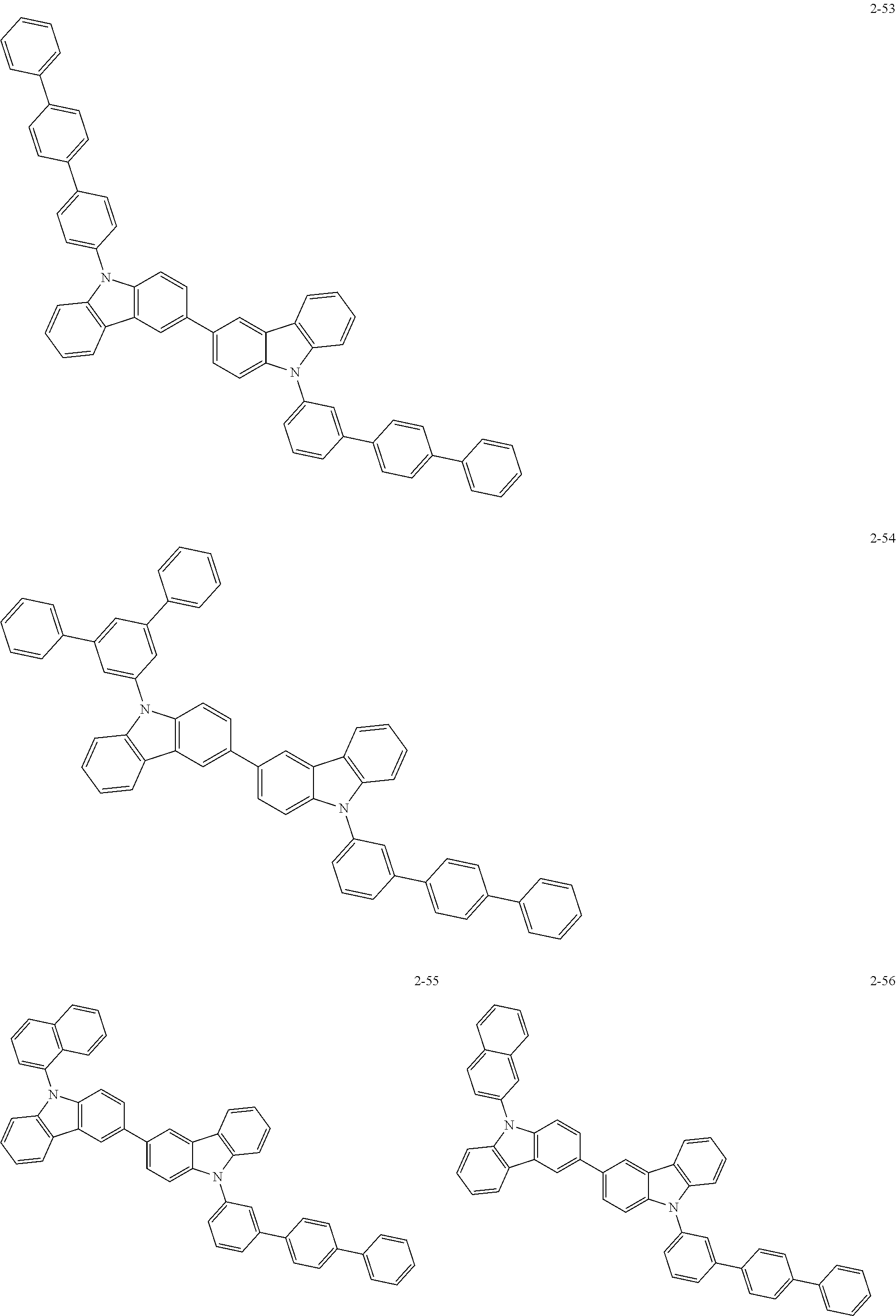 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

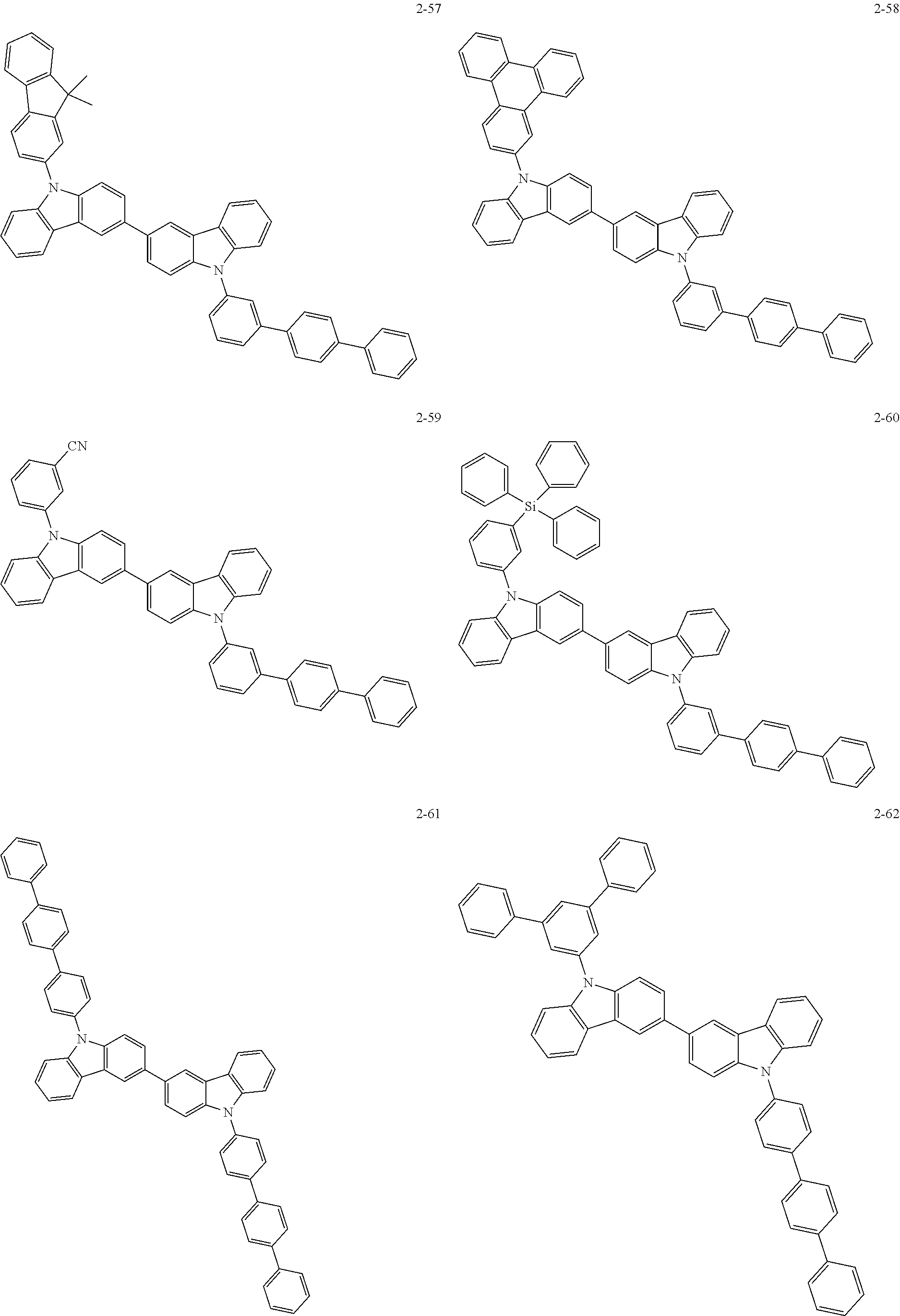

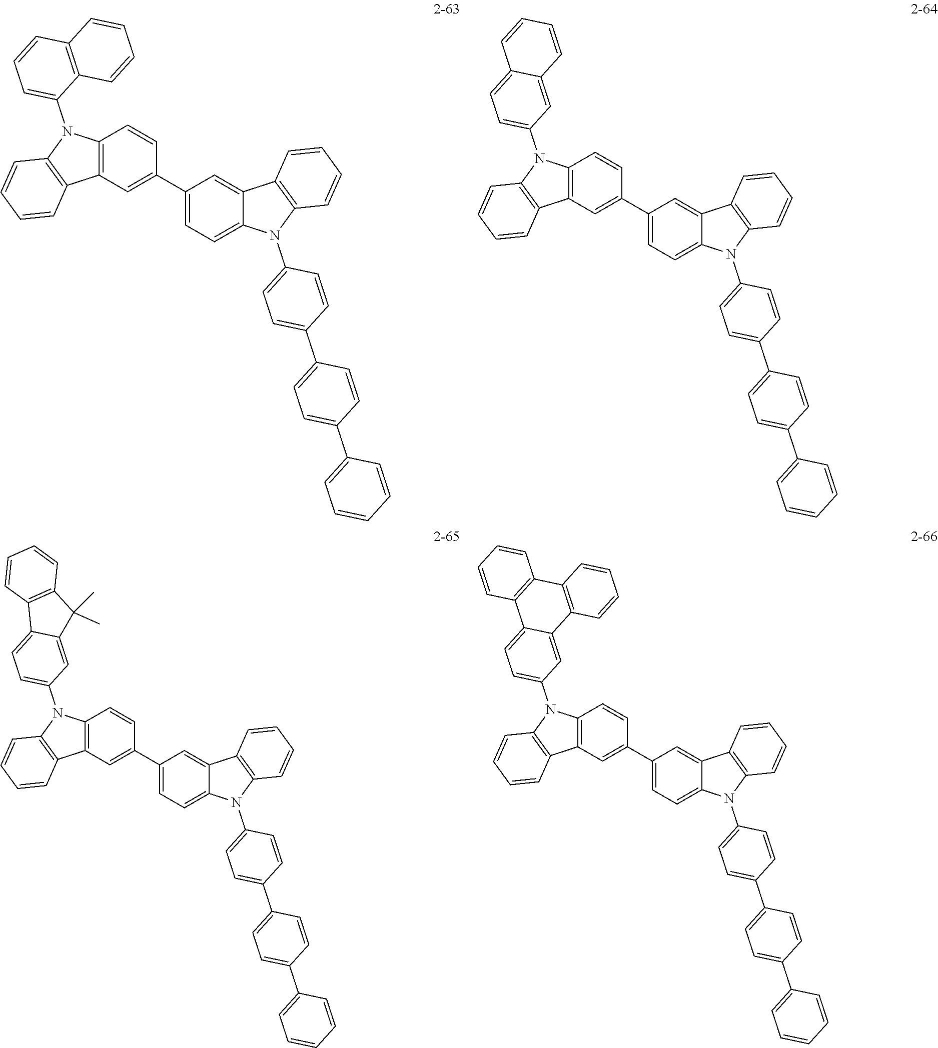 [SMILES: c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1 ; CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1]

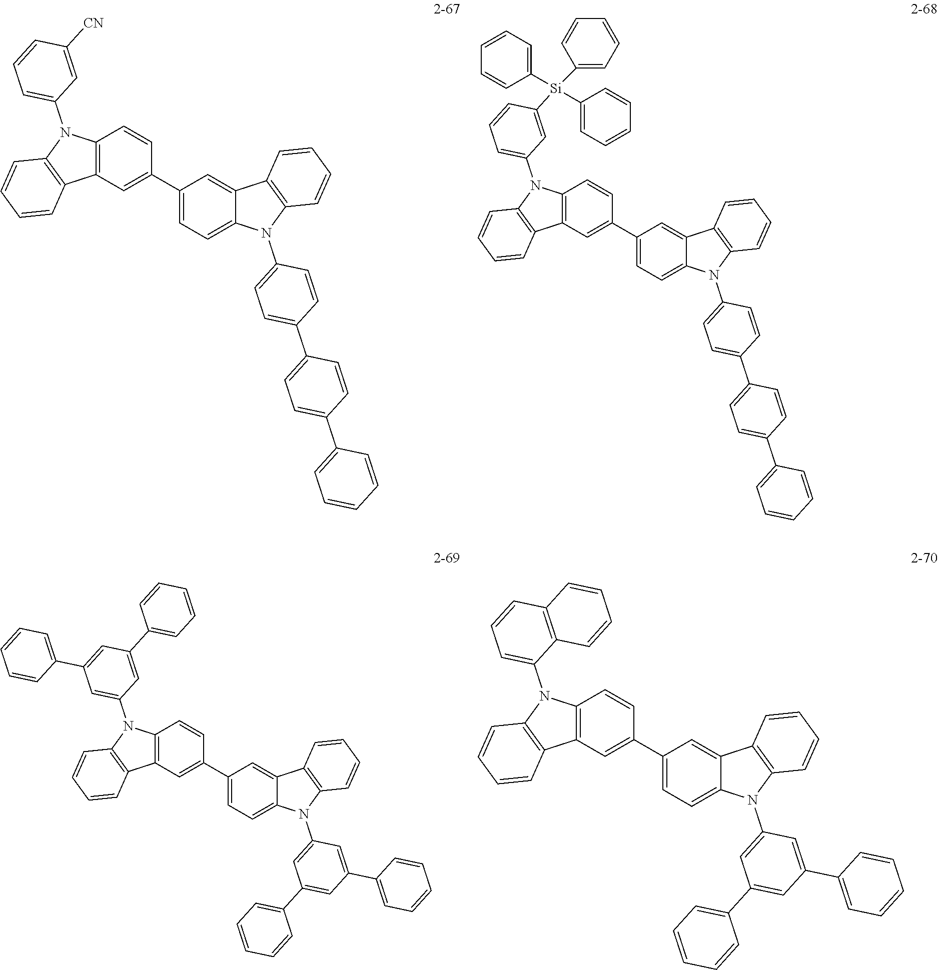 [SMILES: N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

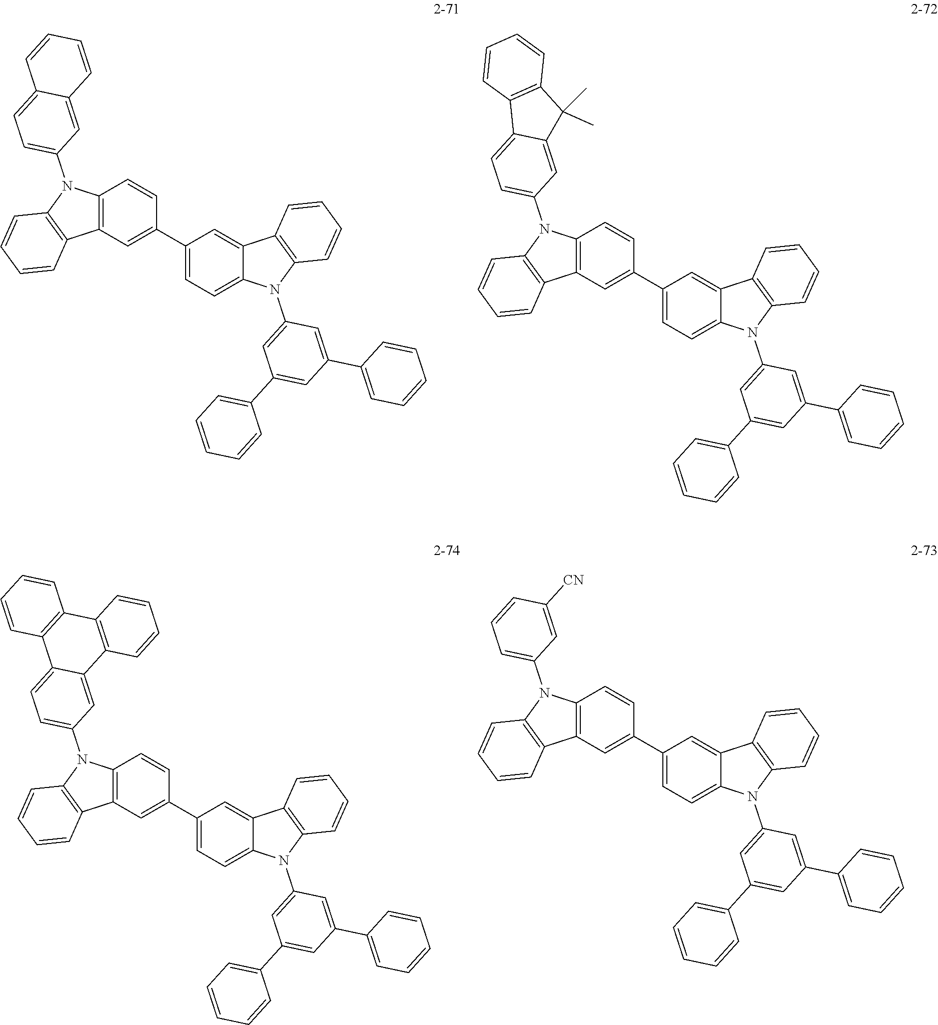 [SMILES: c1ccc(-c2cc(-c3ccccc3)cc(-n3c4ccccc4c4cc(-c5ccc6c(c5)c5ccccc5n6-c5ccc6ccccc6c5)ccc43)c2)cc1 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-c2cc(-c3ccccc3)cc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cc(-c5ccccc5)cc(-c5ccccc5)c4)ccc32)c1]

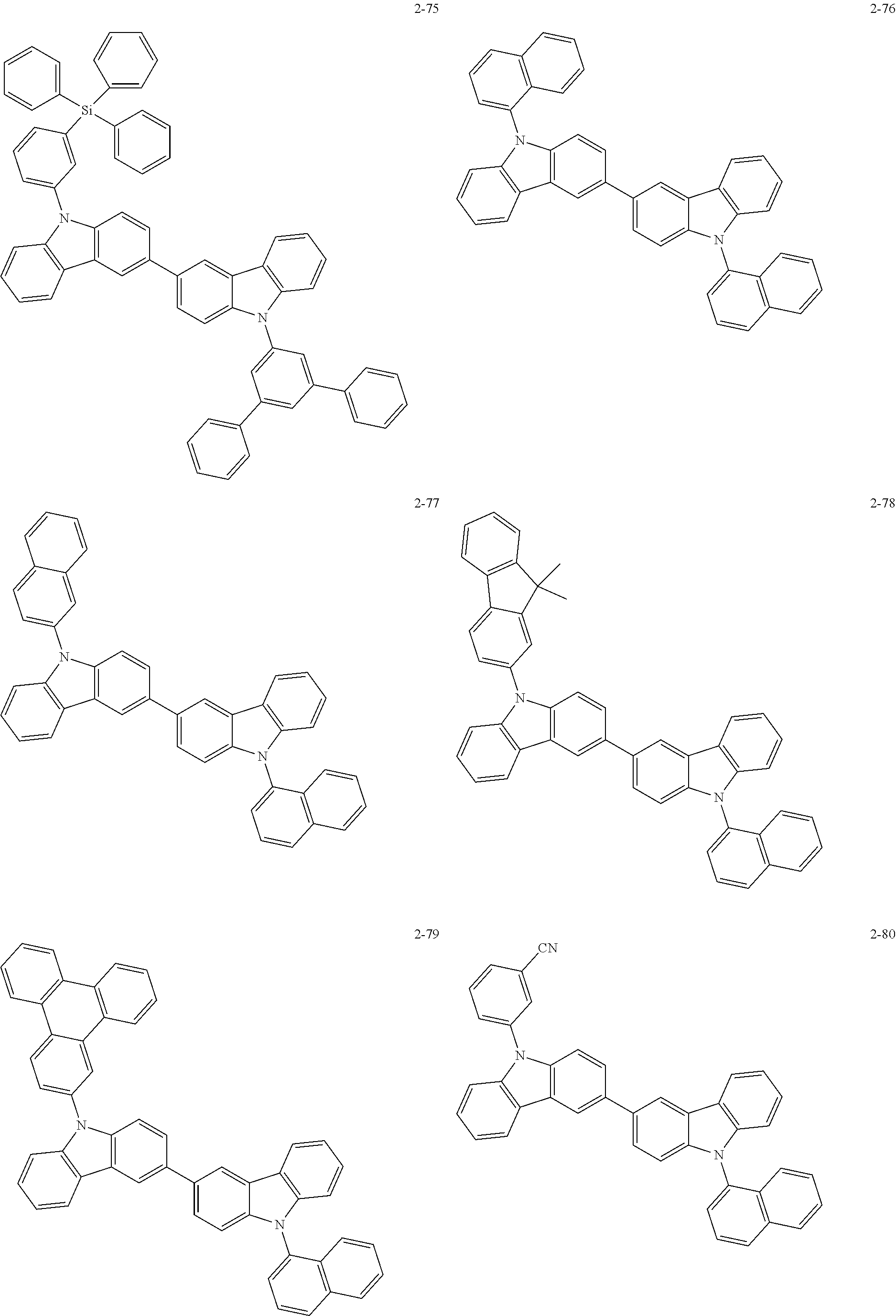

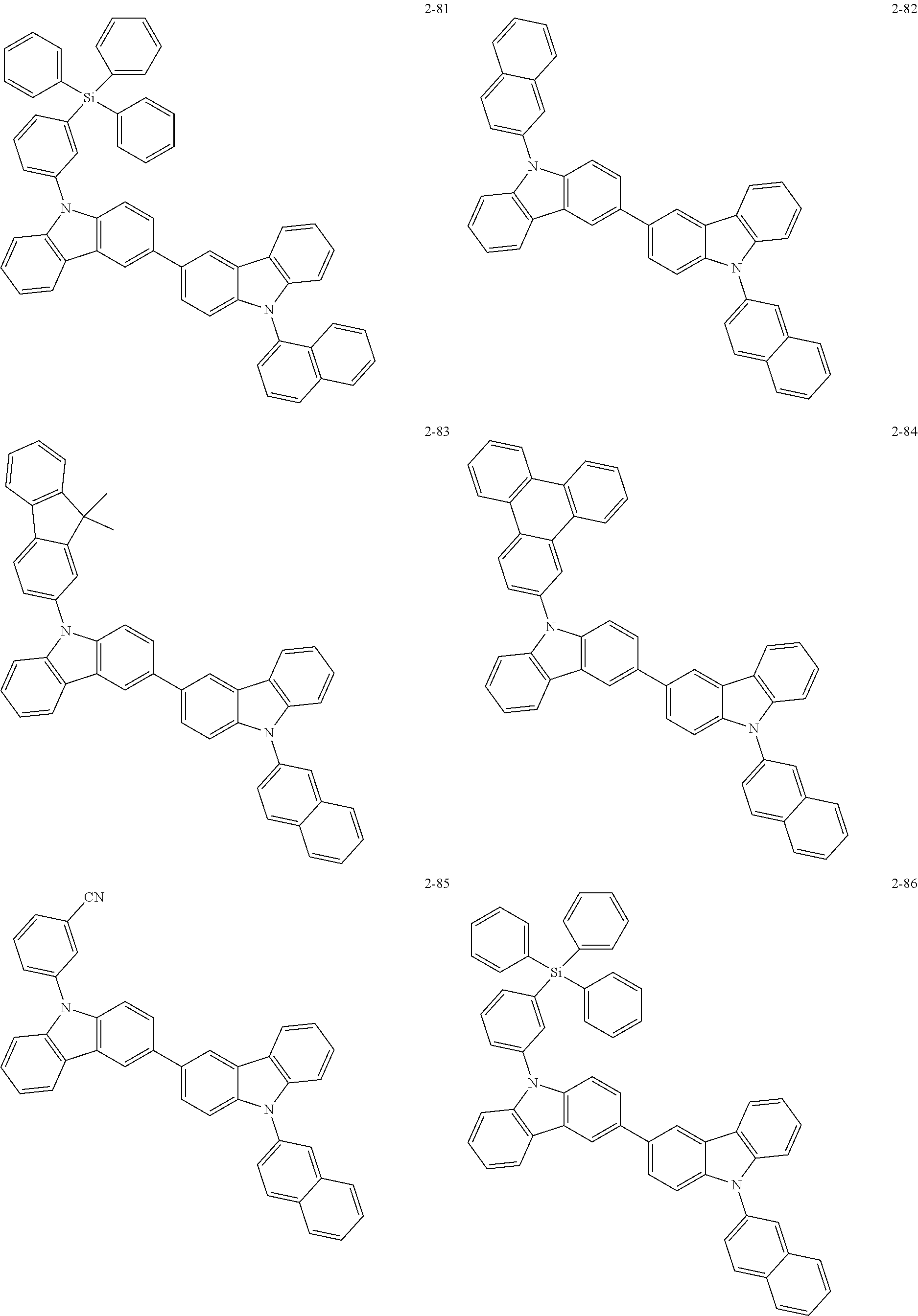

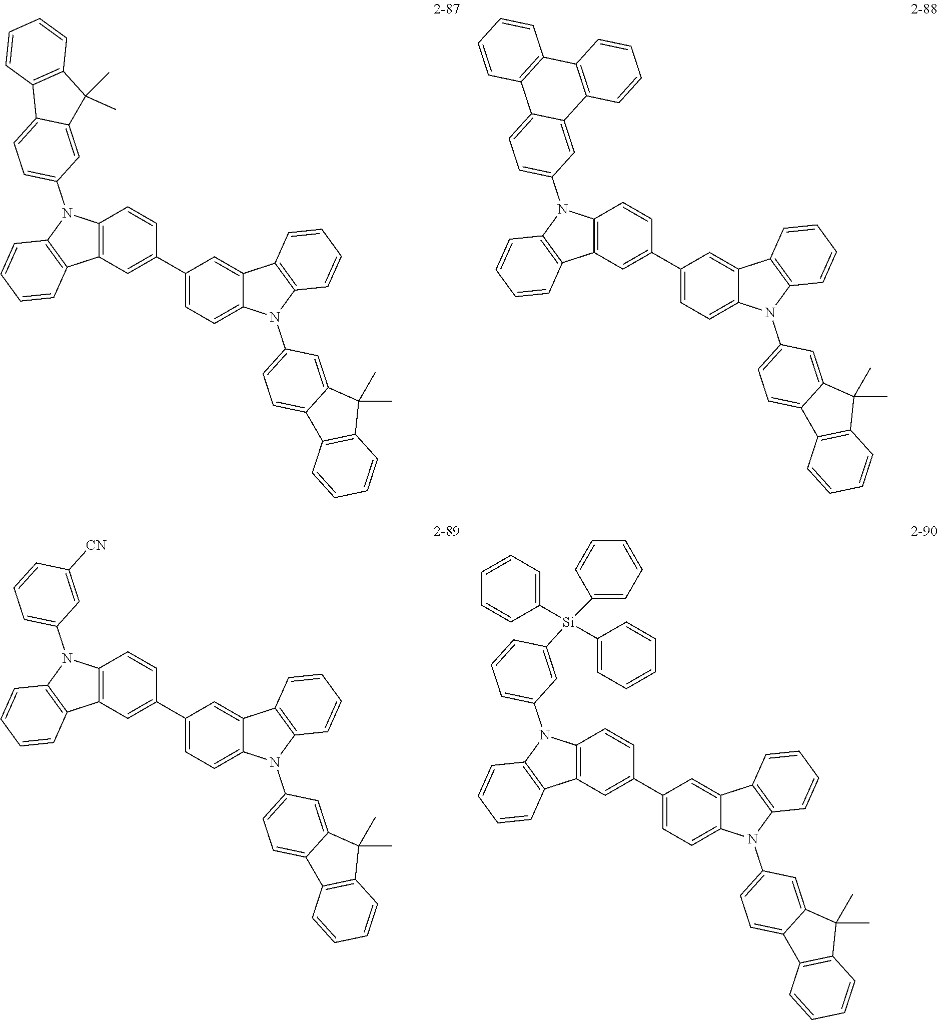 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

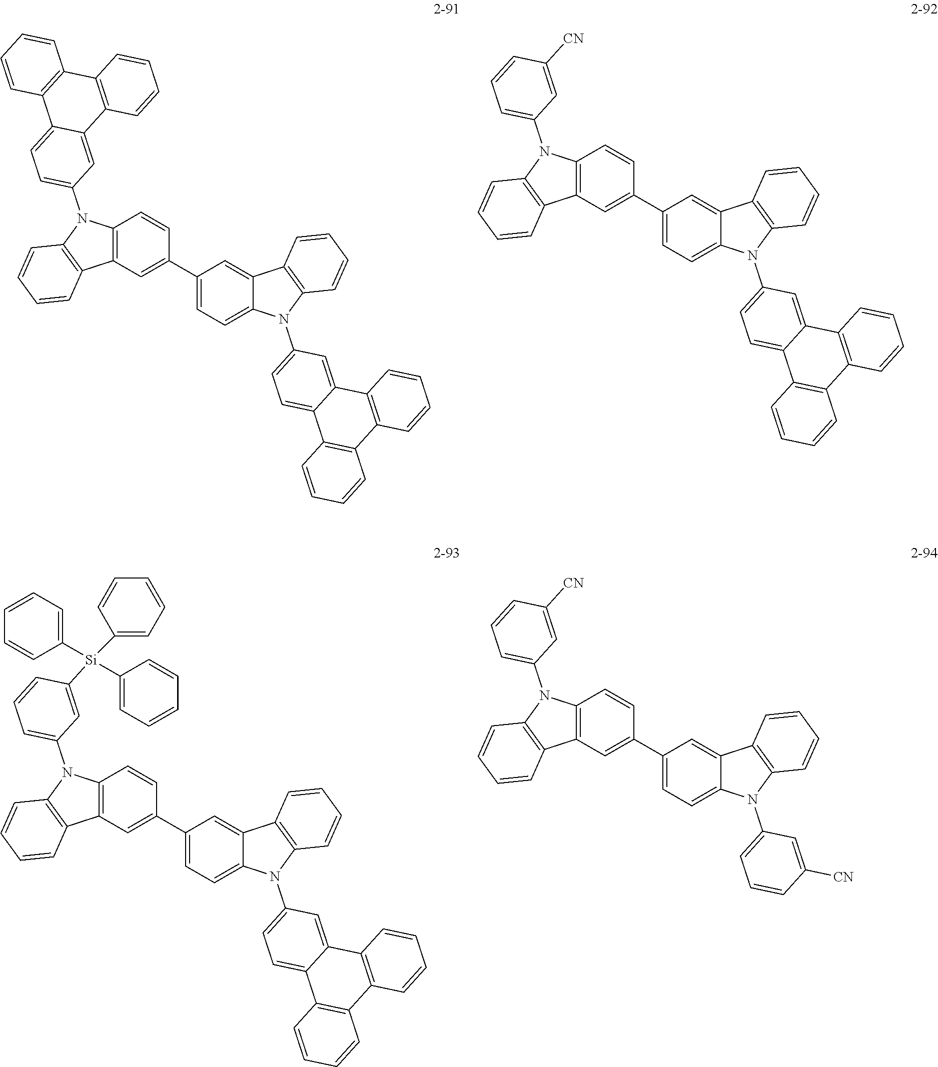 [SMILES: c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1 ; c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1]

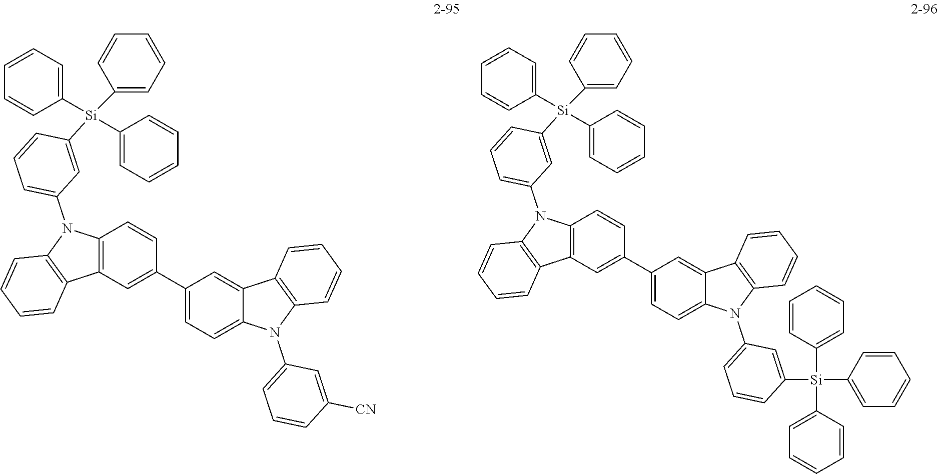 [SMILES: N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1 ; c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

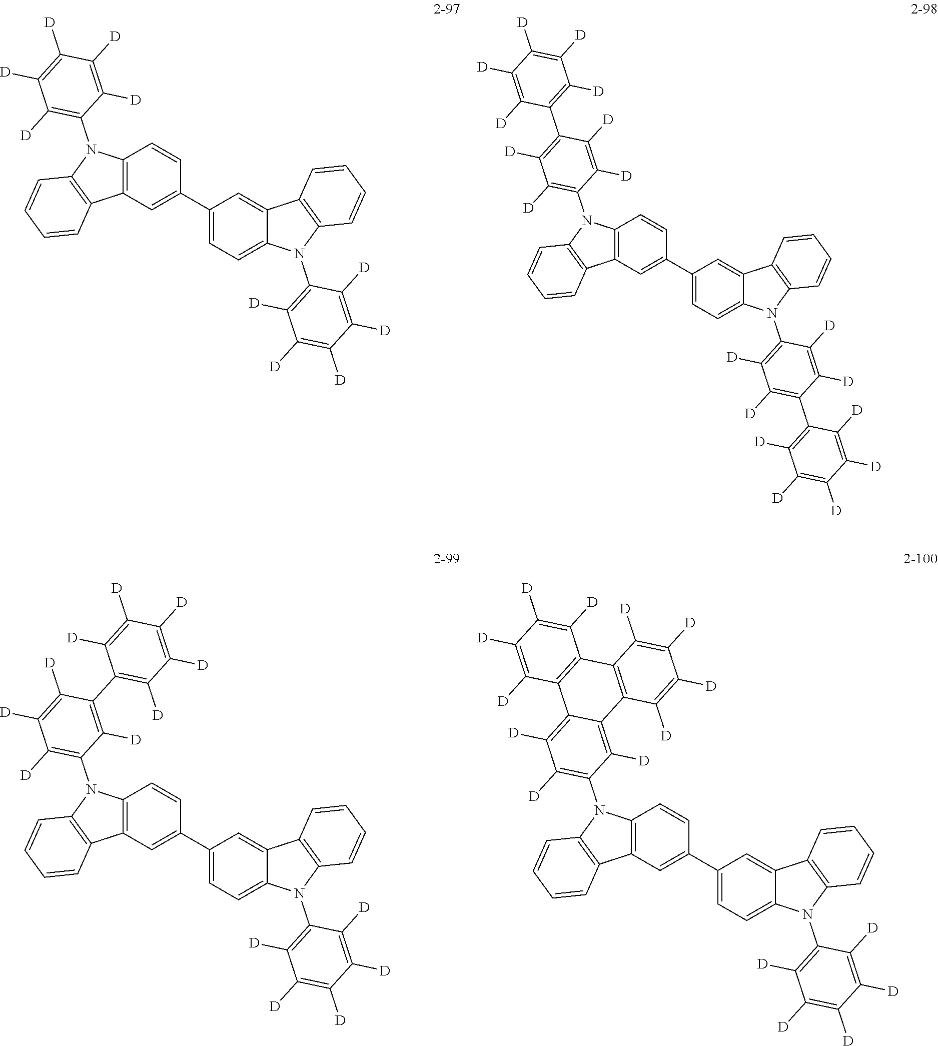

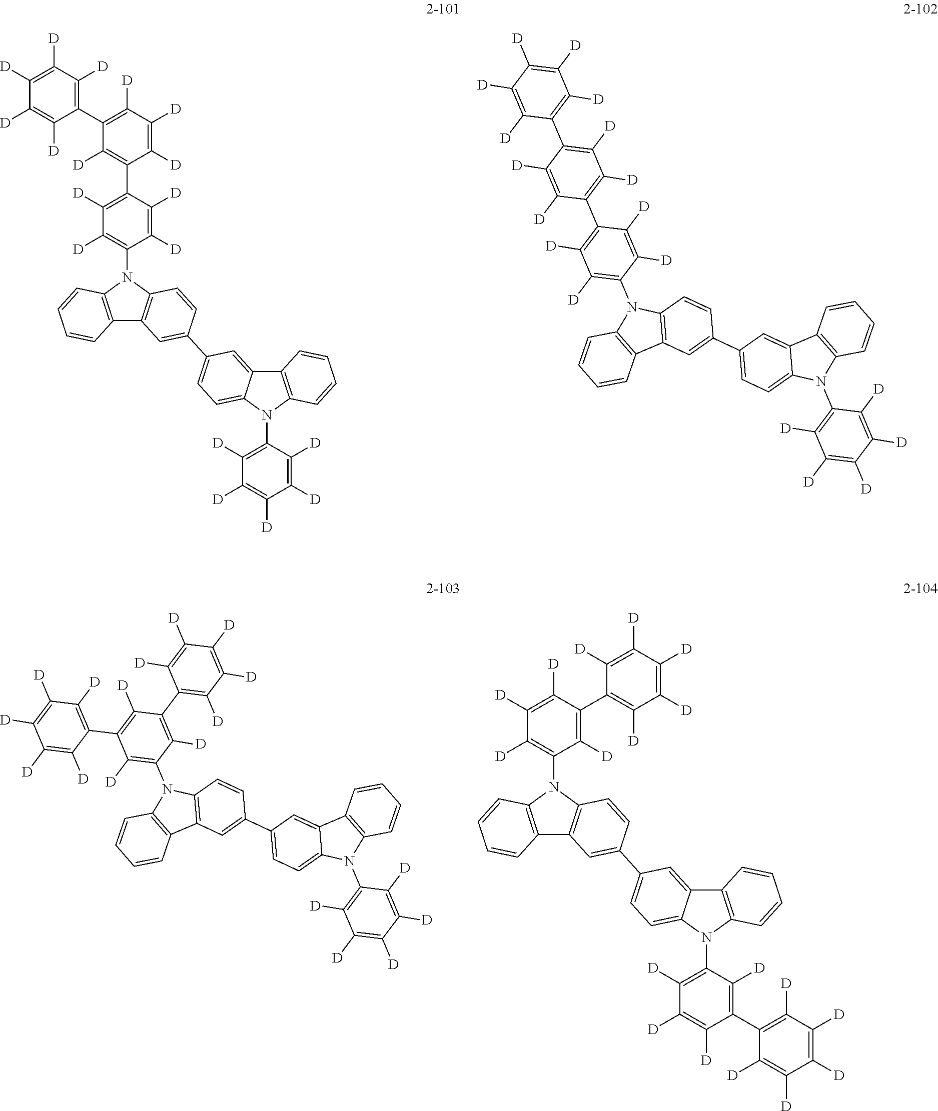

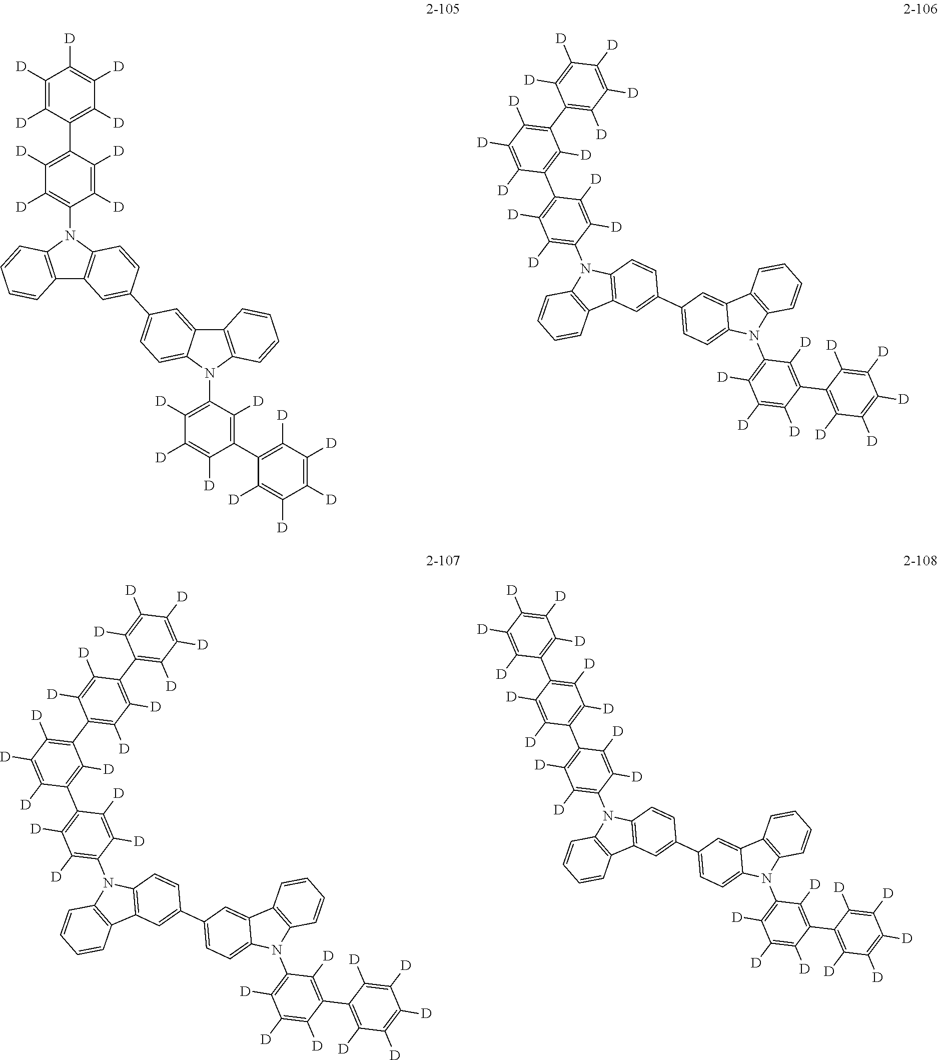

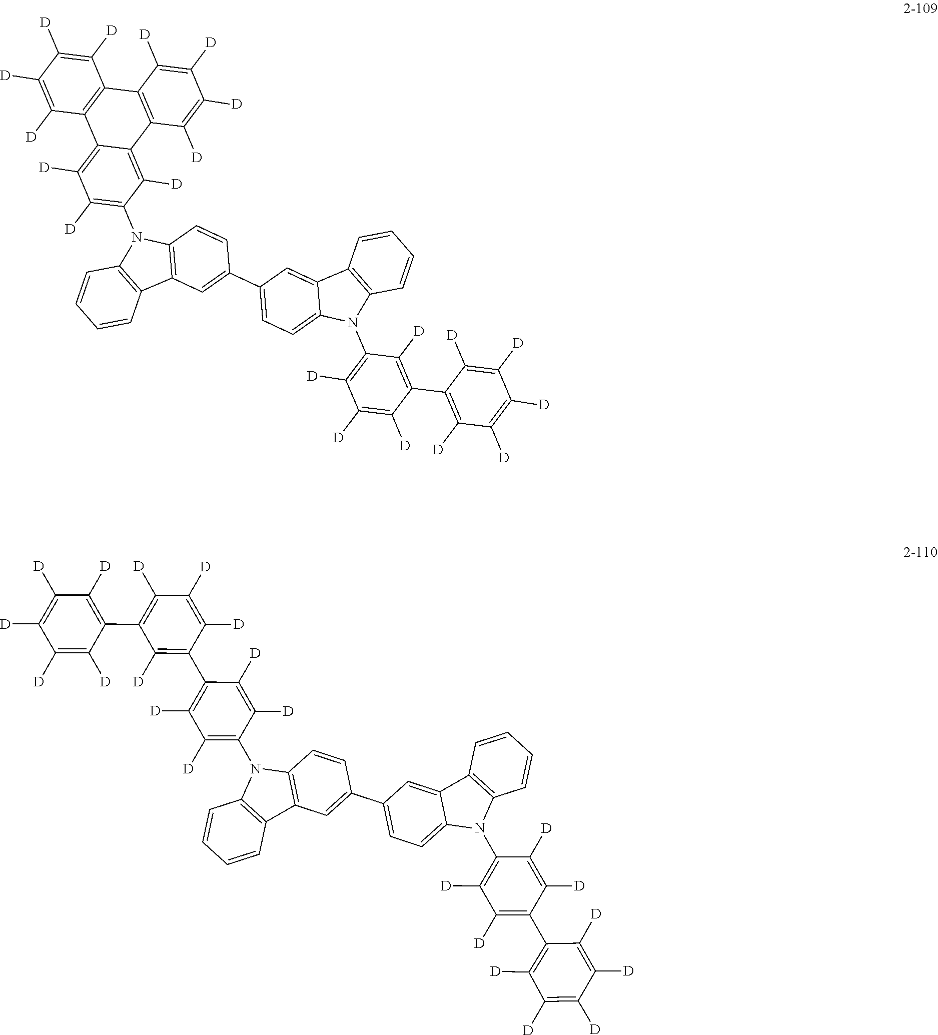 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6c7c([2H])c([2H])c([2H])c([2H])c7c7c([2H])c([2H])c([2H])c([2H])c7c6c5[2H])ccc43)c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c(-c7c([2H])c([2H])c([2H])c([2H])c7[2H])c6[2H])c([2H])c5[2H])ccc43)c([2H])c2[2H])c([2H])c1[2H]]

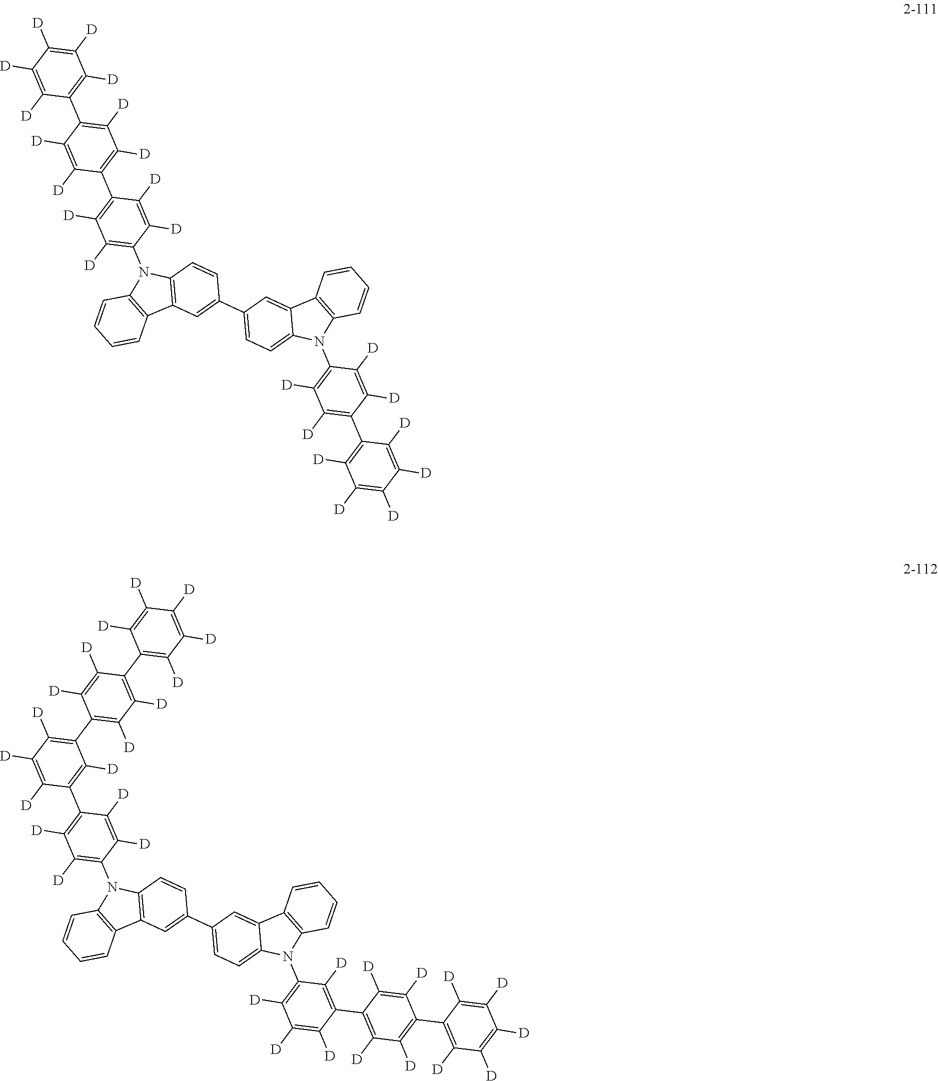

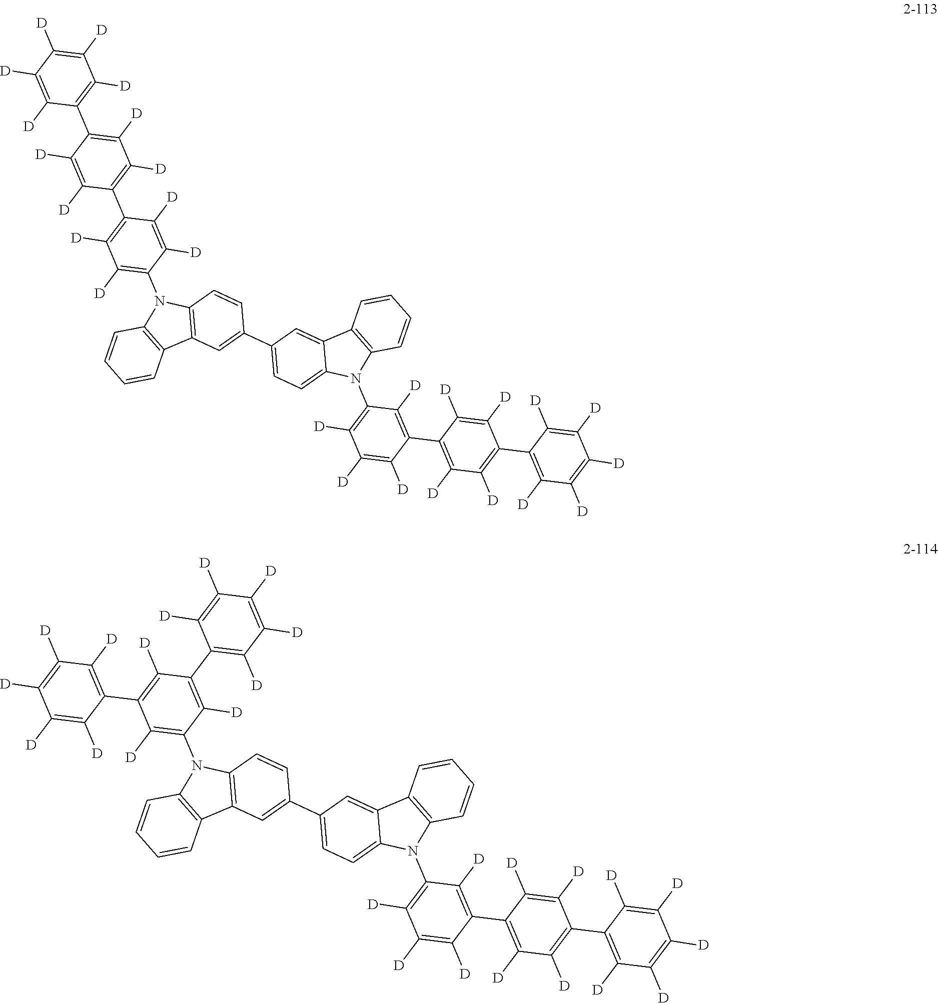

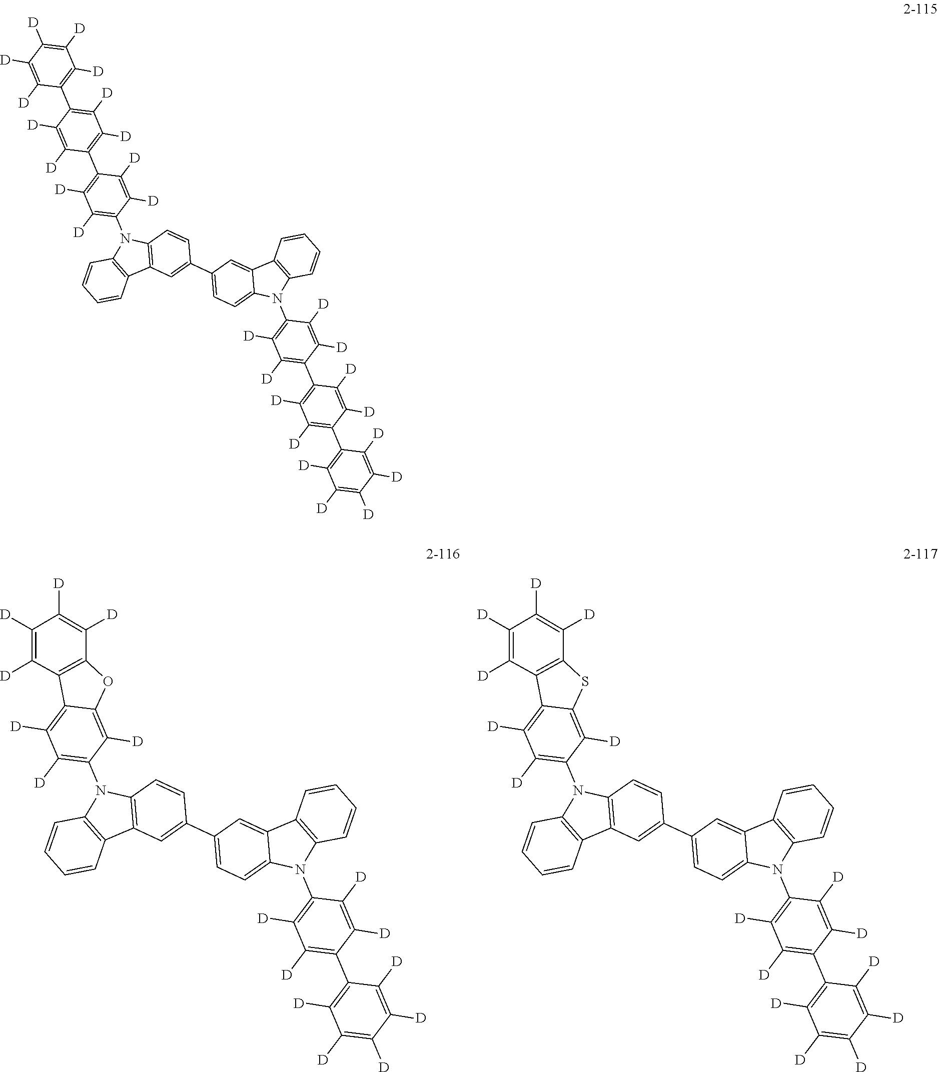

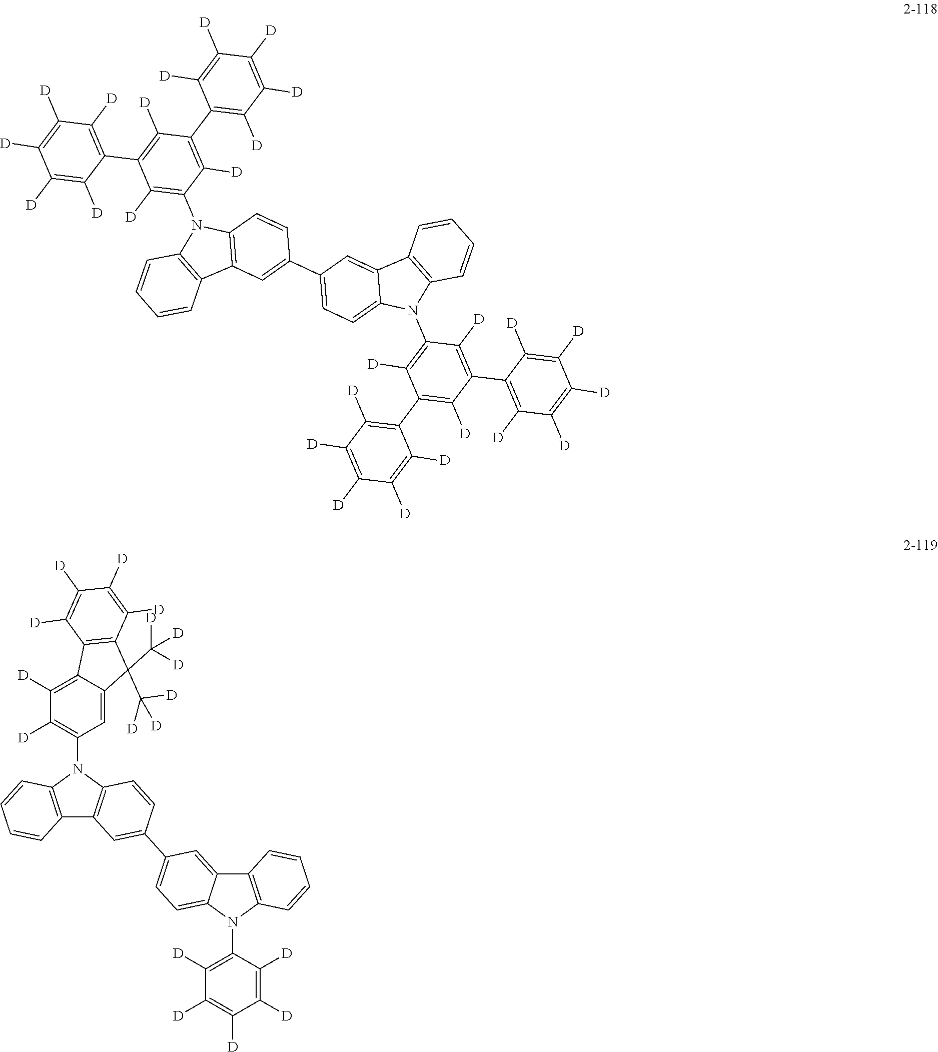 [SMILES: [2H]c1c([2H])c([2H])c(-c2c([2H])c(-c3c([2H])c([2H])c([2H])c([2H])c3[2H])c([2H])c(-n3c4ccccc4c4cc(-c5ccc6c(c5)c5ccccc5n6-c5c([2H])c(-c6c([2H])c([2H])c([2H])c([2H])c6[2H])c([2H])c(-c6c([2H])c([2H])c([2H])c([2H])c6[2H])c5[2H])ccc43)c2[2H])c([2H])c1[2H] ; [2H]c1c(-n2c3ccccc3c3cc(-c4ccc5c(c4)c4ccccc4n5-c4c([2H])c([2H])c([2H])c([2H])c4[2H])ccc32)cc2c(c1[2H])-c1c([2H])c([2H])c([2H])c([2H])c1C2(C([2H])([2H])[2H])C([2H])([2H])[2H]]

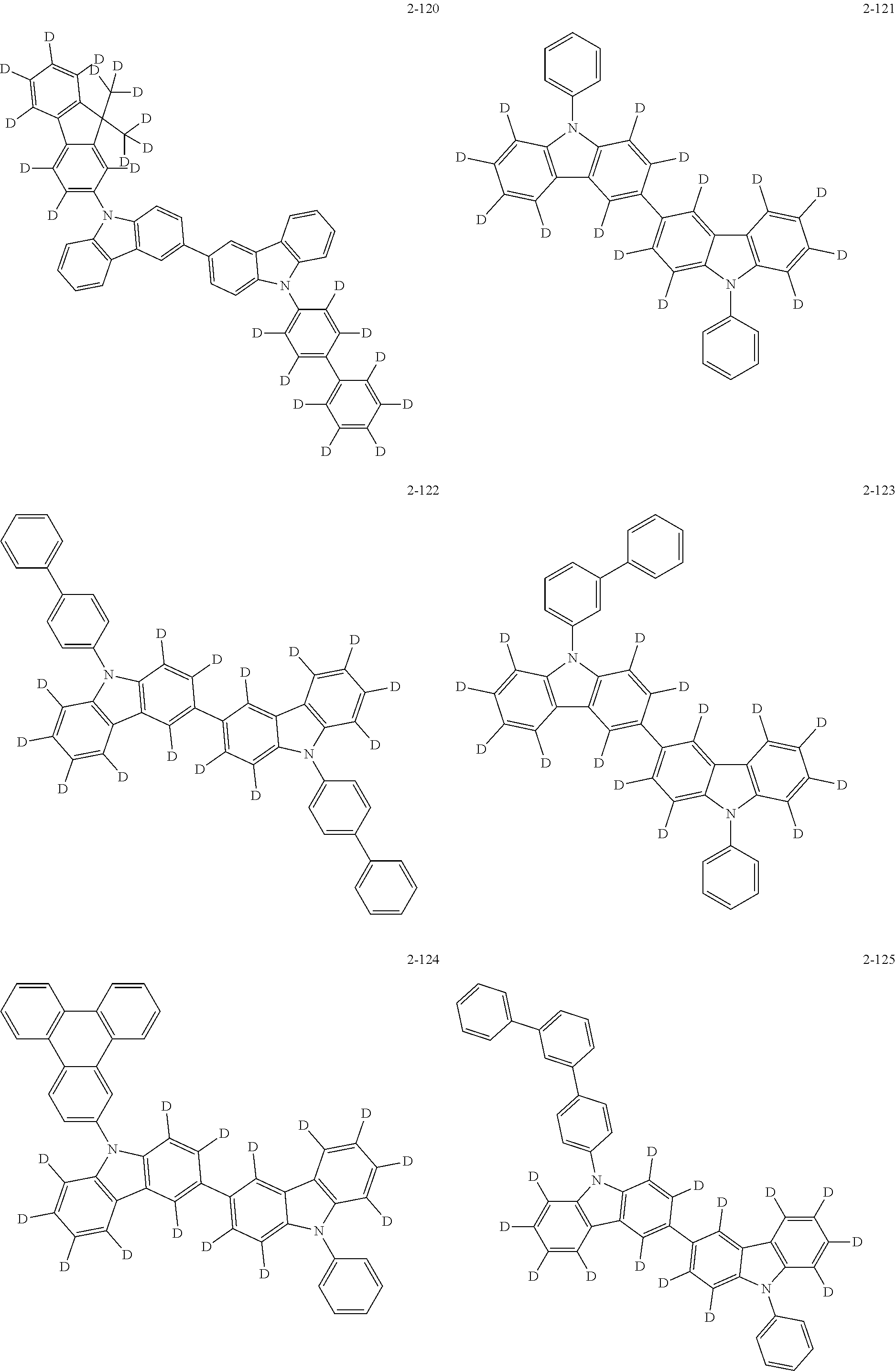

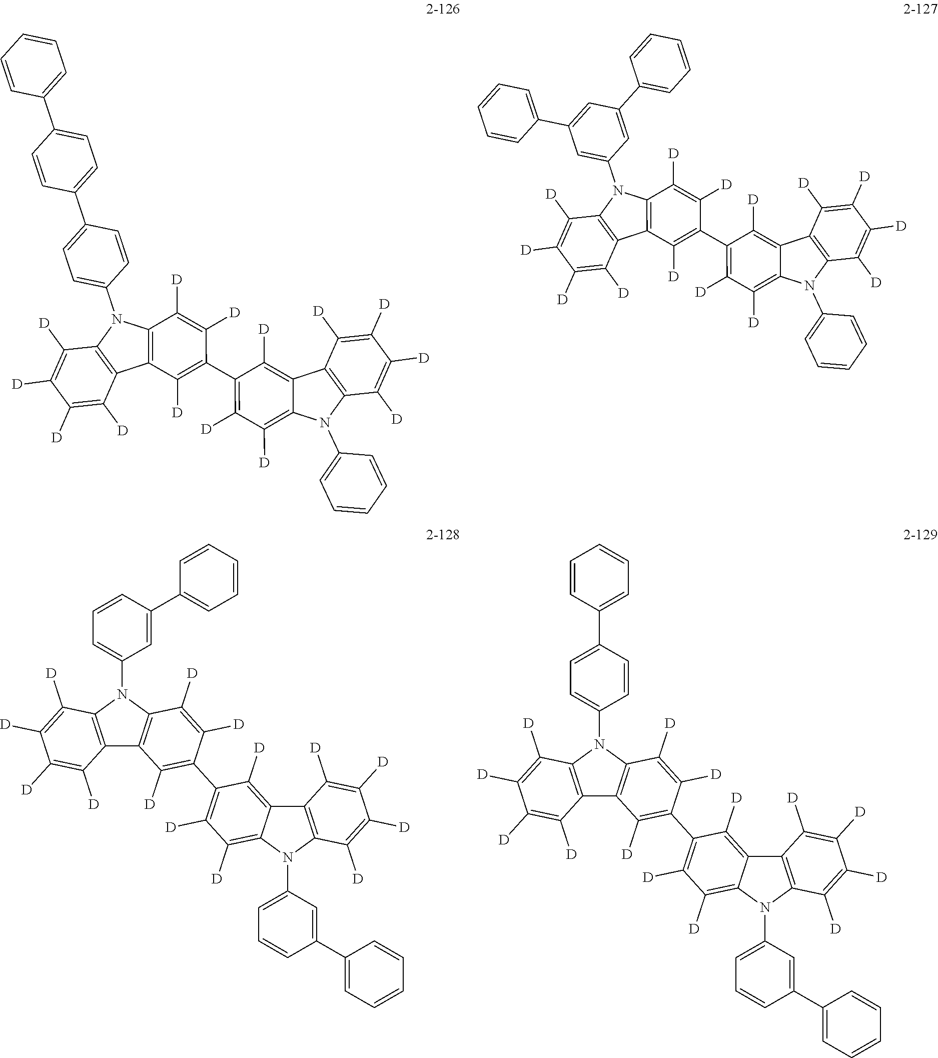

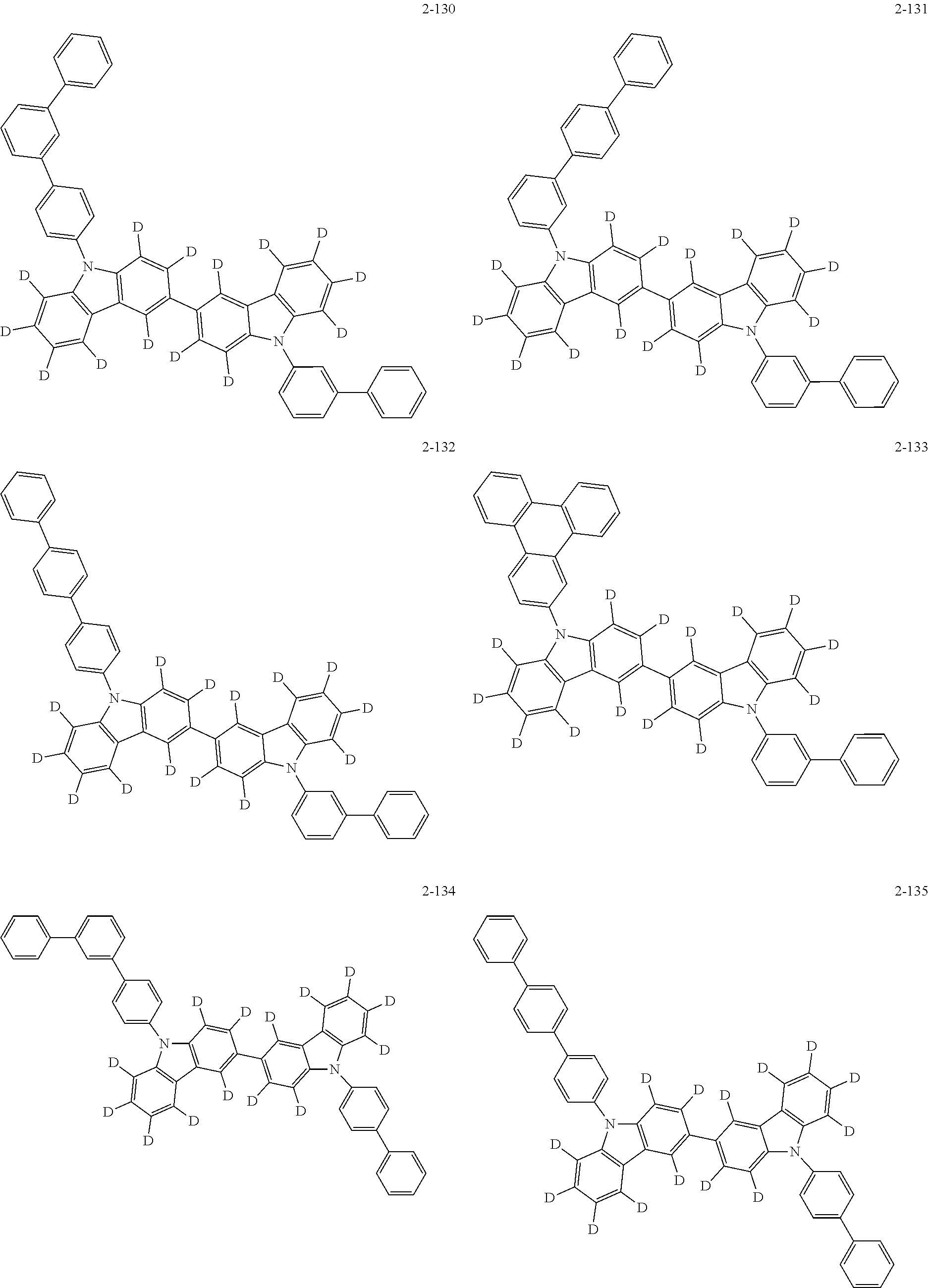

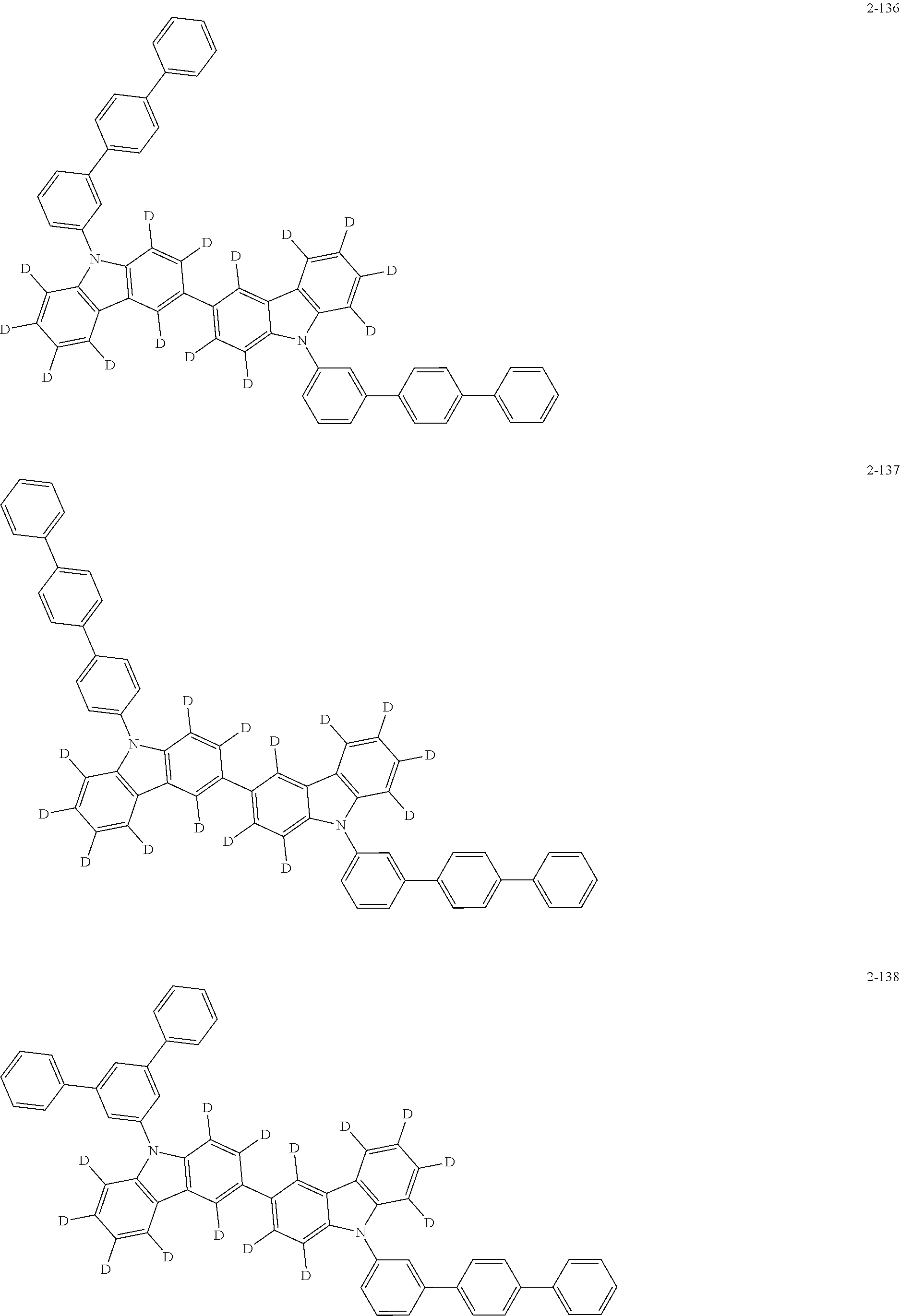

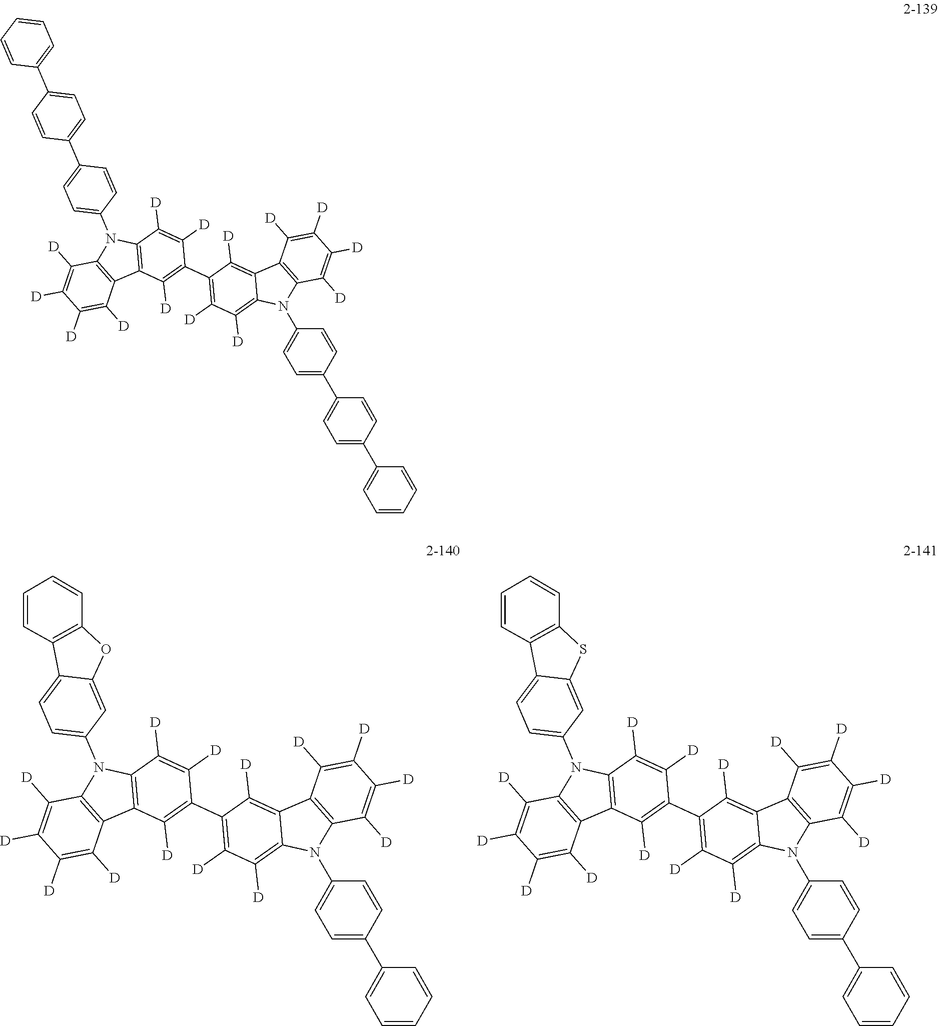

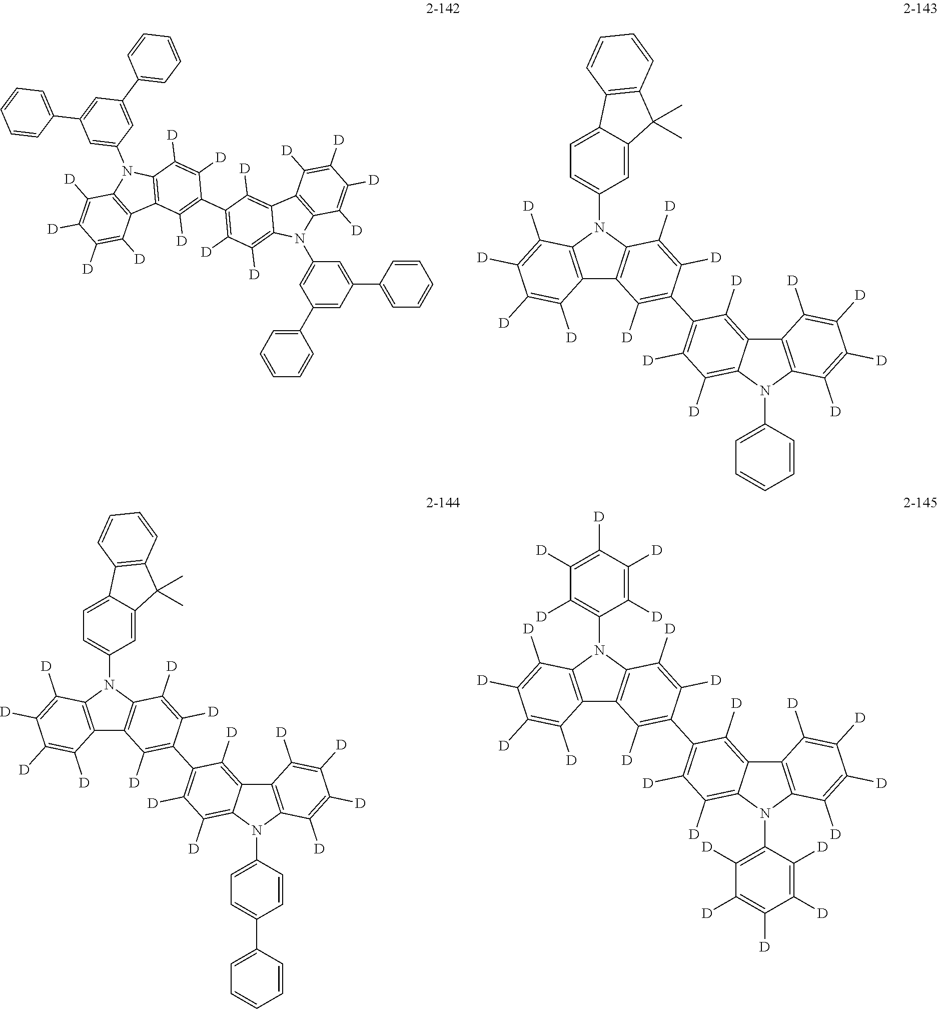

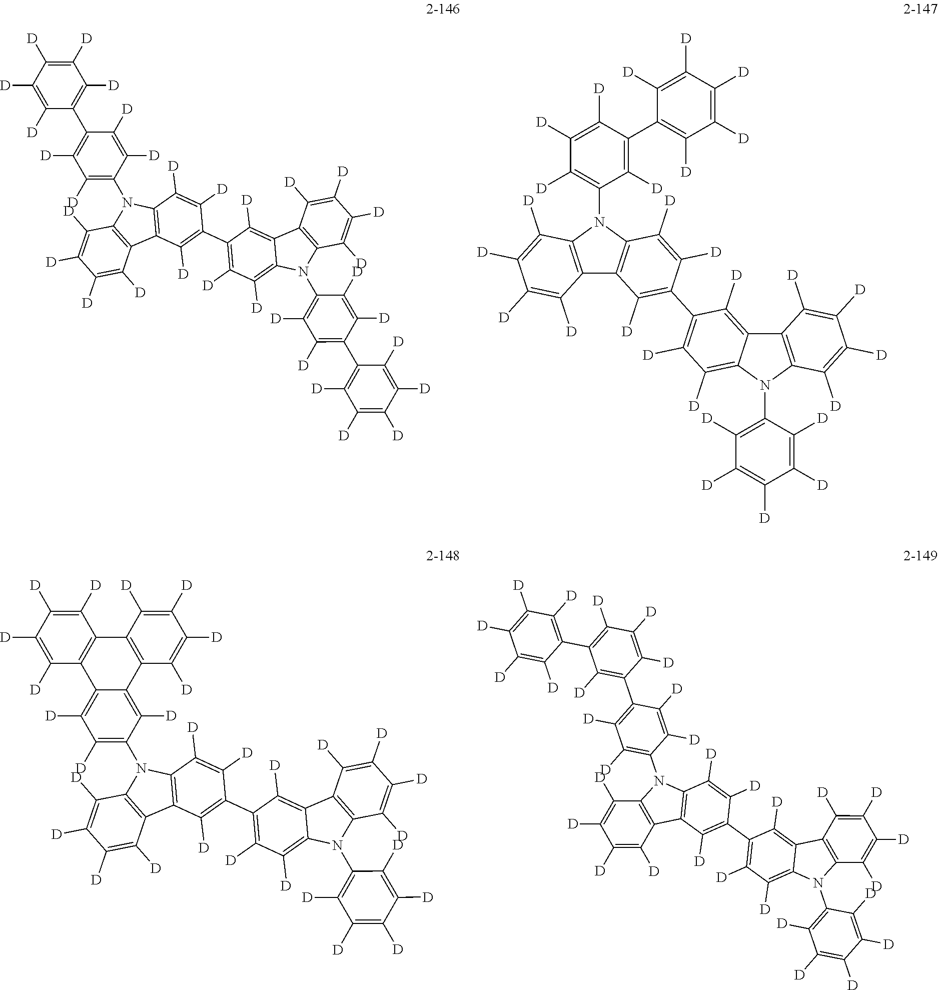

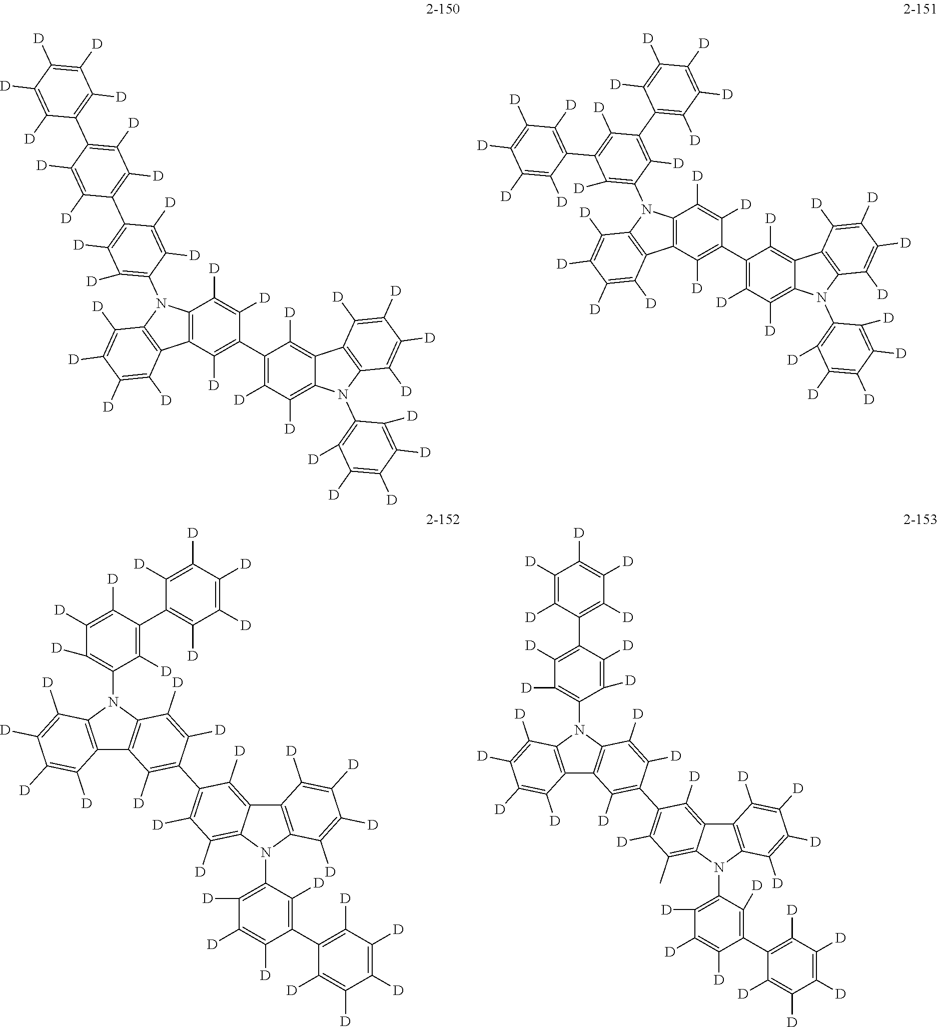

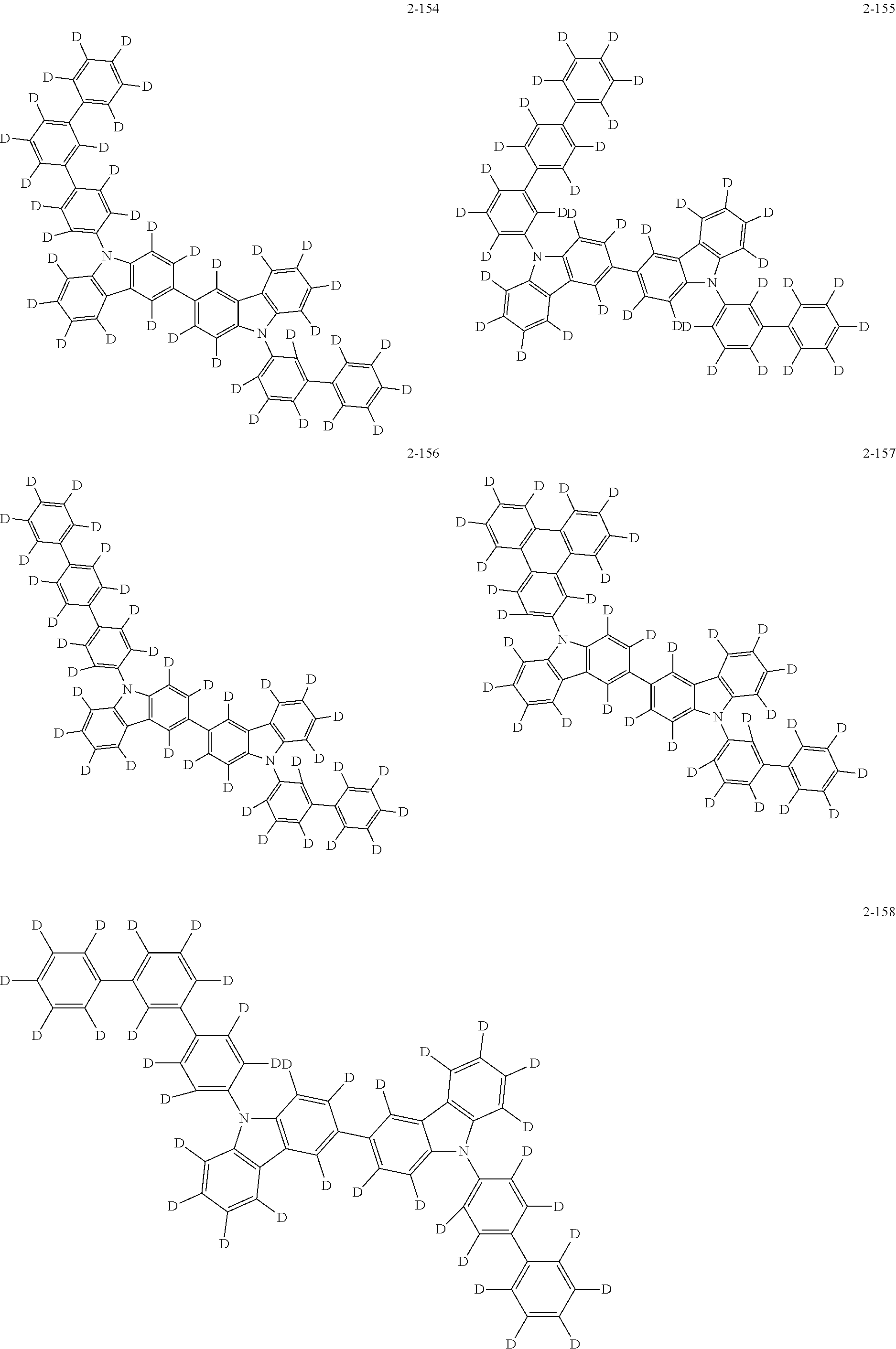

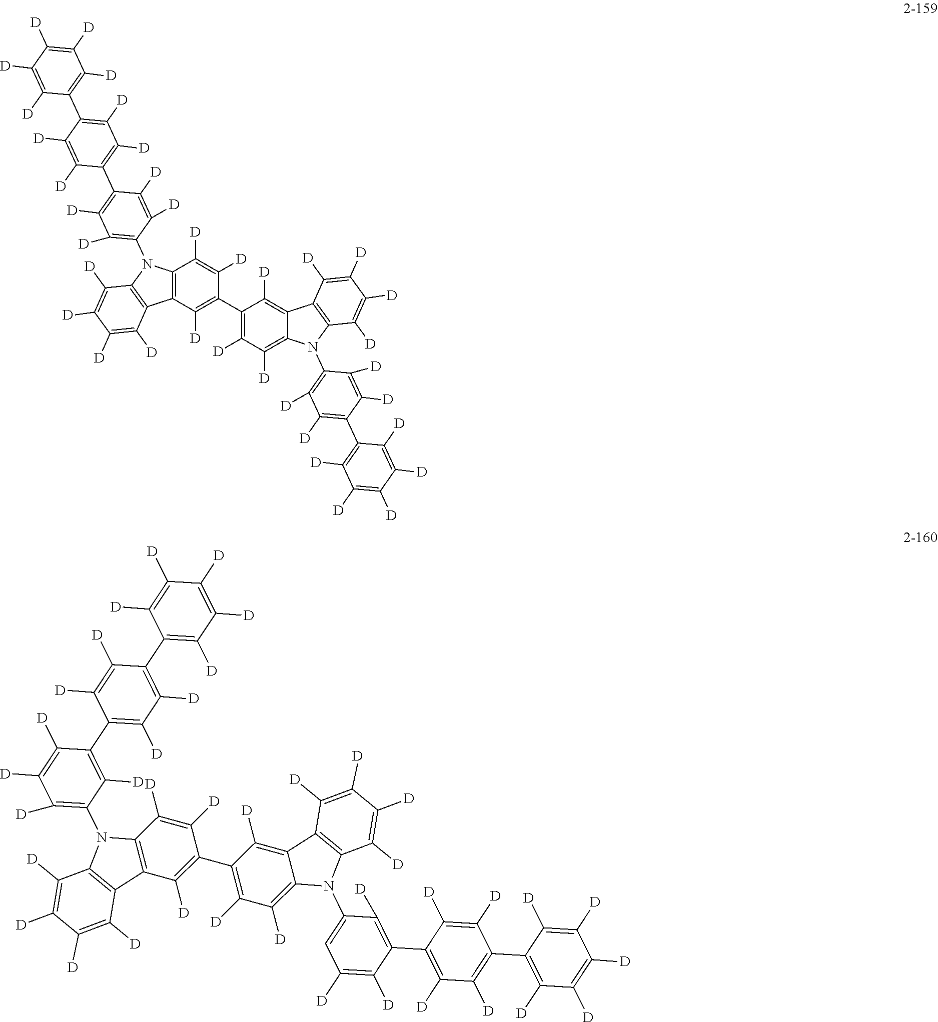

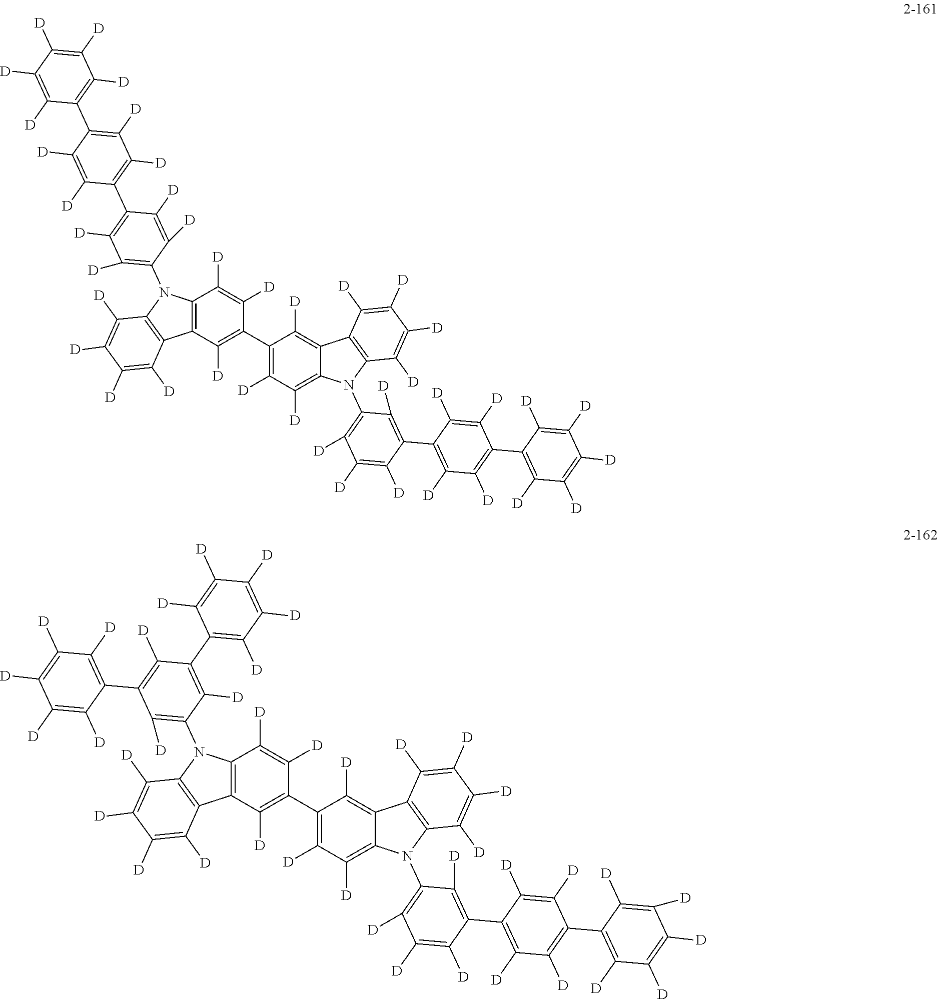

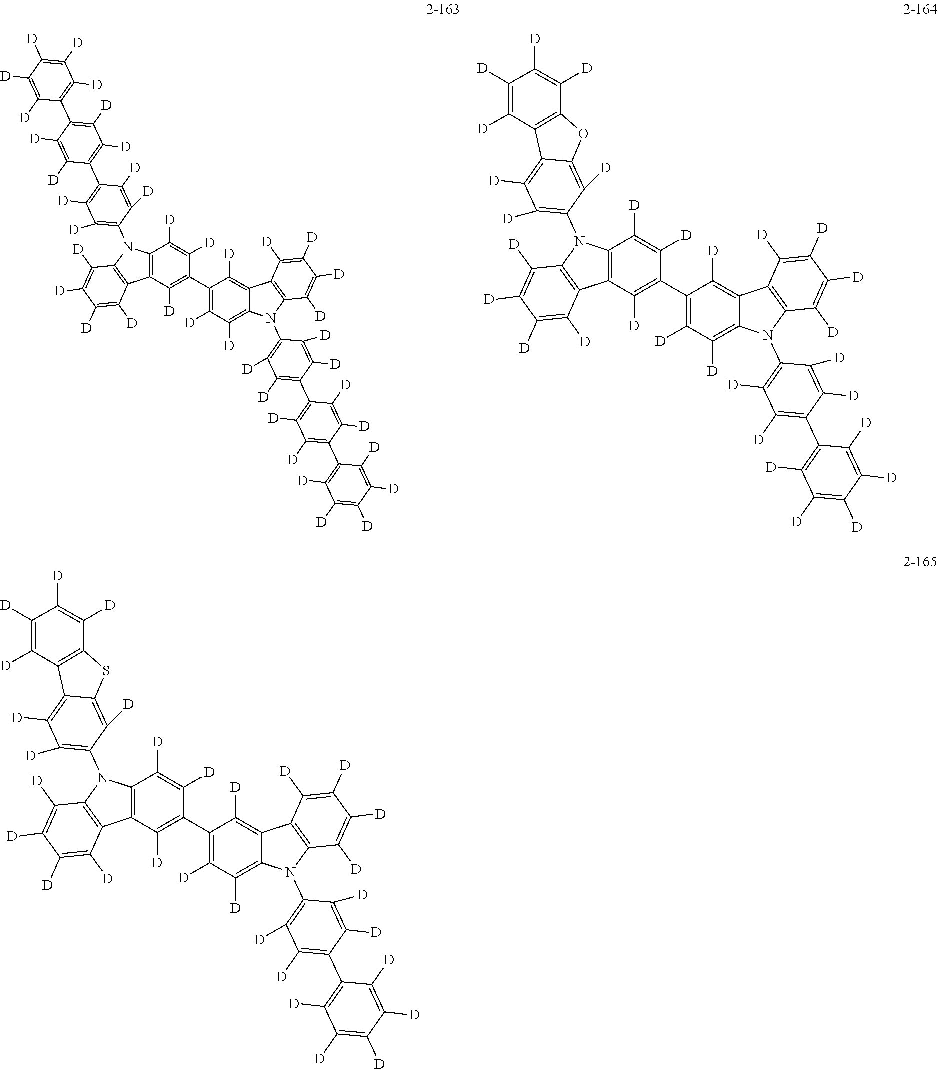

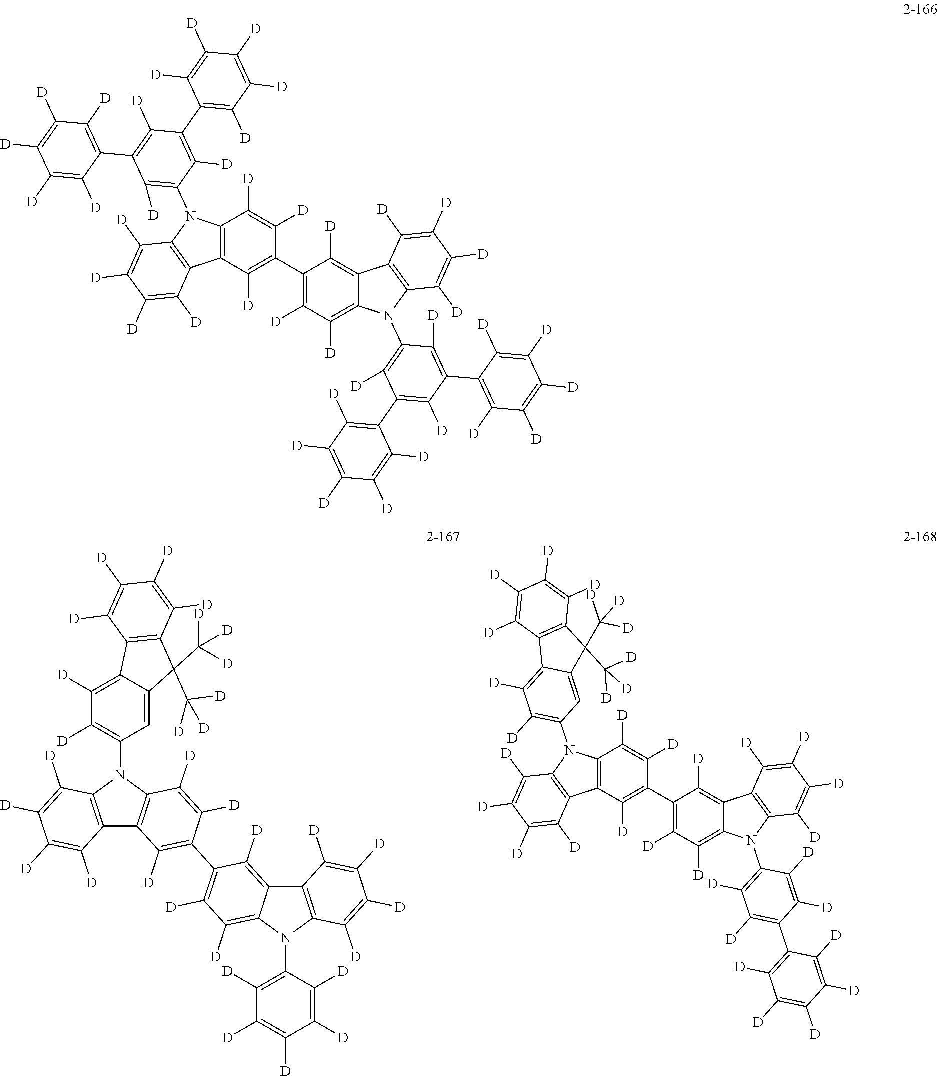

10. The organic light emitting device of claim 8, wherein a deuterium content of Chemical Formula 2 is 0%, 100%, or 10% to 80%.

11. The organic light emitting device of claim 7, wherein the organic material layer comprises a light emitting layer, and the light emitting layer comprises the heterocyclic compound of Chemical Formula 1.

12. The organic light emitting device of claim 7, wherein the organic material layer comprises a light emitting layer, the light emitting layer comprises a host material, and the host material comprises the heterocyclic compound.

13. The organic light emitting device of claim 7, further comprising one or two or more layers selected from the group consisting of a light emitting layer, a hole injection layer, a hole transport layer, an electron injection layer, an electron transport layer, an electron blocking layer and a hole blocking layer.

14. A composition for an organic material layer of an organic light emitting device, the composition comprising the heterocyclic compound according to claim 1, and a heterocyclic compound represented by the following Chemical Formula 2: 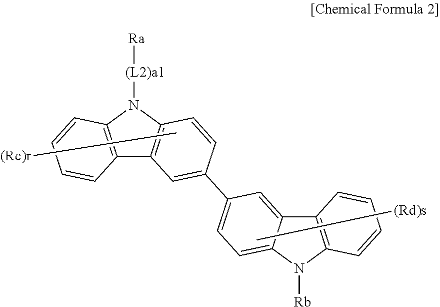
wherein, in Chemical Formula 2,
Rc and Rd are the same as or different from each other, and are each independently selected from the group consisting of hydrogen; deuterium; a halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —SiRR′R″; —P(═O)RR′; and —NRR′, or two or more adjacent groups are bonded to each other to form a substituted or unsubstituted aromatic hydrocarbon ring or a substituted or unsubstituted hetero ring,
L2 is a direct bond; a substituted or unsubstituted C6 to C60 arylene group; or a substituted or unsubstituted C2 to C60 heteroarylene group,
Ra and Rb are the same as or different from each other, and are each independently —CN; —SiRR′R″; a substituted or unsubstituted C6 to C60 aryl group; or a substituted or unsubstituted C2 to C60 heteroaryl group,
R, R′ and R″ are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
a1 is an integer from 0 to 4,
r and s are an integer from 0 to 7, and
when a1, s and r are 2 or higher, substituents in the parenthesis are the same as or different from each other.

15. The composition of claim 14, wherein a weight ratio of the heterocyclic compound:the heterocyclic compound represented by Chemical Formula 2 in the composition is 1:10 to 10:1.
